SEQUENCE LISTING (248 sequences)

SEQ ID NO: 1 - protein, 293 aa, Klebsiella oxytoca
MTMRQCAIYGKGGIGKSTTTQNLVAALAEMGKKVMIVGCDPKADSTRLILHAKAQNTIMEMAAEVGSVEDLELEDVLQIGYGDVRCAESGGPEPGVGCAGRGVITAINFLEEEGAYEDDLDFVFYDVLGDVVCGGFAMPIRENKAQEIYIVCSGEMMAMYAANNISKGIVKYAKSGKVRLGGLICNSRQTDREDELIIALAEKLGTQMIHFVPRDNIVQRAEIRRMTVIEYDPACKQANEYRTLAQKIVNNTMKVVPTPCTMDELESLLMEFGIMEEEDTSIIGKTAAEENAA

SEQ ID NO: 2 - protein, 483 aa, Klebsiella oxytoca
MMTNATGERNLALIQEVLEVFPETARKERRKHMMVSDPKMKSVGKCIISNRKSQPGVMTVRGCAYAGSKGVVFGPIKDMAHISHGPAGCGQYSRAERRNYYTGVSGVDSFGTLNFTSDFQERDIVFGGDKKLSKLIEEMELLFPLTKGITIQSECPVGLIGDDISAVANASSKALDKPVIPVRCEGFRGVSQSLGHHIANDVVRDWILNNREGQPFETTPYDVAIIGDYNIGGDAWASRILLEEMGLRVVAQWSGDGTLVEMENTPFVKLNLVHCYRSMNYIARHMEEKHQIPWMEYNFFGPTKIAESLRKIADQFDDTIRANAEAVIARYEGQMAAIIAKYRPRLEGRKVLLYIGGLRPRHVIGAYEDLGMEIIAAGYEFAHNDDYDRTLPDLKEGTLLFDDASSYELEAFVKALKPDLIGSGIKEKYIFQKMGVPFRQMHSWDYSGPYHGYDGFAIFARDMDMTLNNPAWNELTAPWLKSA

SEQ ID NO: 3 - protein, 520 aa, Klebsiella oxytoca
MSQTIDKINSCYPLFEQDEYQELFRNKRQLEEAHDAQRVQEVFAWTTTAEYEALNFQREALTVDPAKACQPLGAVLCSLGFANTLPYVHGSQGCVAYFRTYFNRHFKEPIACVSDSMTEDAAVFGGNNNMNLGLQNASALYKPEIIAVSTTCMAEVIGDDLQAFIANAKKDGFVDSSIAVPHAHTPSFIGSHVTGWDNMFEGFAKTFTADYQGQPGKLPKLNLVTGFETYLGNFRVLKRMMEQMAVPCSLLSDPSEVLDTPADGHYRMYSGGTTQQEMKEAPDAIDTLLLQPWQLLKSKKVVQEMWNQPATEVAIPLGLAATDELLMTVSQLSGKPIADALTLERGRLVDMMLDSHTWLHGKKFGLYGDPDFVMGLTRFLLELGCEPTVILSHNANKRWQKAMNKMLDASPYGRDSEVFINCDLWHFRSLMFTRQPDFMIGNSYGKFIQRDTLAKGKAFEVPLIRLGFPLFDRHHLHRQTTWGYEGAMNIVTTLVNAVLEKLDSDTSQLGKTDYSFDLVR

SEQ ID NO: 4 - protein, 468 aa, Klebsiella oxytoca
MTSCSSFSGGKACRPADDSALTPLVADKAAAHPCYSRHGHHRFARMHLPVAPACNLQCNYCNRKFDCSNESRPGVSSTLLTPEQAVVKVRQVAQAIPQLSVVGIAGPGDPLANIARTFRTLELIREQLPDLKLCLSTNGLVLPDAVDRLLDVGVDHVTVTINTLDAEIAAQIYAWLWLDGERYSGREAGEILIARQLEGVRRLTAKGVLVKINSVLIPGINDSGMAGVSRALRASGAFIHNIMPLIARPEHGTVFGLNGQPEPDAETLAATRSRCGEVMPQMTHCHQCRADAIGMLGEDRSQQFTQLPAPESLPAWLPILHQRAQLHASIATRGESEADDACLVAVASSRGDVIDCHFGHADRFYIYSLSAAGMVLVNERFTPKYCQGRDDCEPQDNAARFAAILELLADVKAVFCVRIGHTPWQQLEQEGIEPCVDGAWRPVSEVLPAWWQQRRGSWPAALPHKGVA

SEQ ID NO: 5 - protein, 457 aa, Klebsiella oxytoca
MKGNEILALLDEPACEHNHKQKSGCSAPKPGATAAGCAFDGAQITLLPIADVAHLVHGPIGCAGSSWDNRGSASSGPTLNRLGFTTDLNEQDVIMGRGERRLFHAVRHIVTRYHPAAVFIYNTCVPAMEGDDLEAVCQAAQTATGVPVIAIDAAGFYGSKNLGNRPAGDVMVKRVIGQREPAPWPESTLFAPEQRHDIGLIGEFNIAGEFWHIQPLLDELGIRVLGSLSGDGRFAEIQTMHRAQANMLVCSRALINVARALEQRYGTPWFEGSFYGIRATSDALRQLAALLGDDDLRQRTEALIAREEQAAELALQPWREQLRGRKALLYTGGVKSWSVVSALQDLGMTVVATGTRKSTEEDKQRIRELMGEEAVMLEEGNARTLLDVVYRYQADLMIAGGRNMYTAYKARLPFLDINQEREHAFAGYQGIVTLARQLCQTINSPIWPQTHSRAPWR

SEQ ID NO: 6 - protein, 176 aa, Klebsiella oxytoca
MANIGIFFGTDTGKTRKIAKMIHKQLGELADAPVNINRTTLDDFMAYPVLLLGTPTLGDGQLPGLEAGCESESWSEFISGLDDASLKGKTVALFGLGDQRGYPDNFVSGMRPLFDALSARGAQMIGSWPNEGYEFSASSALEGDRFVGLVLDQDNQFDQTEARLASWLEEIKRTVL

SEQ ID NO: 7 - protein, 1171 aa, Klebsiella oxytoca
MSGKMKTMDGNAAAAWISYAFTEVAAIYPITPSTPMAENVDEWAAQGKKNLFGQPVRLMEMQSEAGAAGAVHGALQAGALTTTYTASQGLLLMIPNMYKIAGELLPGVFHVSARALATNSLNIFGDHQDVMAVRQTGCAMLAENNVQQVMDLSAVAHLAAIKGRIPFVNFFDGFRTSHEIQKIEVLEYEQLATLLDRPALDSFRRNALHPDHPVIRGTAQNPDIYFQEREAGNRFYQALPDIVESYMTQISALTGREYHLFNYTGAADAERVIIAMGSVCDTVQEVVDTLNAAGEKVGLLSVHLFRPFSLAHFFAQLPKTVQRIAVLDRTKEPGAQAEPLCLDVKNAFYHHDDAPLIVGGRYALGGKDVLPNDIAAVFDNLNKPLPMDGFTLGIVDDVTFTSLPPRQQTLAVSHDGITACKFWGMGSDGTVGANKSAIKIIGDKTPLYAQAYFSYDSKKSGGITVSHLRFGDRPINSPYLIHRADFISCSQQSYVERYDLLDGLKPGGTFLLNCSWSDAELEQHLPVGFKRYLARENIHFYTLNAVDIARELGLGGRFNMLMQAAFFKLAAIIDPQTAADYLKQAVEKSYGSKGAAVIEMNQRAIELGMASLHQVTIPAHWATLDEPAAQASAMMPDFIRDILQPMNRQCGDQLPVSAFVGMEDGTFPSGTAAWEKRGIALEVPVWQPEGCTQCNQCAFICPHAAIRPALLNGEEHDAAPVGLLSKPAQGAKEYHYHLAISPLDCSGCGNCVDICPARGKALKMQSLDSQRQMAPVWDYALALTPKSNPFRKTTVKGSQFETPLLEFSGACAGCGETPYARLITQLFGDRMLIANATGCSSIWGASAPSIPYTTNHRGHGPAWANSLFEDNAEFGLGMMLGGQAVRQQIADDMTAALALPVSDELSDAMRQWLAKQDEGEGTRERADRLSERLAAEKEGVPLLEQLWQNRDYFVRRSQWIFGGDGWAYDIGFGGLDHVLASGEDVNILVFDTEVYSNTGGQSSKSTPVAAIAKFAAQGKRTRKKDLGMMAMSYGNVYVAQVAMGADKDQTLRAIAEAEAWPGPSLVIAYAACINHGLKAGMRCSQREAKRAVEAGYWHLWRYHPQREAEGKTPFMLDSEEPEESFRDFLLGEVRYASLHKTTPHLADALFSRTEEDARARFAQYRRLAGEE

SEQ ID NO: 8 - protein, 266 aa, Klebsiella oxytoca
MNPWQRFARQRLARSRWNRDPAALDPADTPAFEQAWQRQCHMEQTIVARVPEGDIPAALLENIAASLAIWLDEGDFAPPERAAIVRHHARLELAFADIARQAPQPDLSTVQAWYLRHQTQFMRPEQRLTRHLLLTVDNDREAVHQRILGLYRQINASRDAFAPLAQRHSHCPSALEEGRLGWISRGLLYPQLETALFSLAENALSLPIASELGWHLLWCEAIRPAAPMEPQQALESARDYLWQQSQQRHQRQWLEQMISRQPGLCG

SEQ ID NO: 9 - protein, 461 aa, Klebsiella oxytoca
MADIFRTDKPLAVSPIKTGQPLGAILASLGIEHSIPLVHGAQGCSAFAKVFFIQHFHDPVPLQSTAMDPTSTIMGADGNIFTALDTLCQRNNPQAIVLLSTGLSEAQGSDISRVVRQFREEYPRHKGVAILTVNTPDFYGSMENGFSAVLESVIEQWVPPAPRPAQRNRRVNLLVSHLCSPGDIEWLRRCVEAFGLQPIILPDLAQSMDGHLAQGDFSPLTQGGTPLRQIEQMGQSLCSFAIGVSLHRASSLLAPRCRGEVIALPHLMTLERCDAFIHQLAKISGRAVPEWLERQRGQLQDAMIDCHMWLQGQRMAIAAEGDLLAAWCDFANSQGMQPGPLVAPTGHPSLRQLPVERVVPGDLEDLQTLLCAHPADLLVANSHARDLAEQFALPLVRAGFPLFDKLGEFRRVRQGYSGMRDTLFELANLIRERHHHLAHYRSPLRQNPESSLSTGGAYAAD

SEQ ID NO: 10 - protein, 167 aa, Artificial Sequence
  Klebsiella sp. RCB570
MPPLDWLRRLWLLYHAGKGSFPLRMGLSPRDWQALRRRLGEVETPLDGETLTRRRLMAELNATREEERQQLGAWLAGWMQQDAGPMAQIIAEVSLAFNHLWQDLGLASRAELRLLMSDCFPQLVVMNEHNMRWKKFFYRQRCLLQQGEVICRSPSCDECWERSACFE

SEQ ID NO: 11 - protein, 400 aa, Klebsiella oxytoca
MKQVYLDNNATTRLDPMVLEAMMPFLTDFYGNPSSIHDFGIPAQAALERAHQQAAALLGAEYPSEIIFTSCATEATATAIASAIALLPERREIITSVVEHPATLAACEHMEREGYRIHRIAVDGEGALDMAQFRAALSPRVALVSVMWANNETGVLFPIGEMAELAHEQGALFHCDAVQVVGKIPIAVGQTRIDMLSCSAHKFHGPKGVGCLYLRRGTRFRPLLRGGHQEYGRRAGTENICGIVGMGAACELANIHLPGMTHIGQLRNRLEHRLLASVPSVMVMGGGQPAVPGTVNLAFEFIEGEAILLLLNQAGIAASSGSACTSGSLEPSHVMRAMNIPYTAAHGTIRFSLSRYTREKEIDYVVATLPPIIDRLRALSPYWQNGKPRPADAVFTPVYG

SEQ ID NO: 12 - protein, 548 aa, Klebsiella oxytoca
MWNYSEKVKDHFFNPRNARVVDNANAVGDVGSLSCGDALRLMLRVDPQSEIIEEAGFQTFGCGSAIASSSALTELIIGHTLAEAGQITNQQIADYLDGLPPEKMHCSVMGQEALRAAIANFRGESLEEEHDEGKLICKCFGVDEGHIRRAVQNNGLTTLAEVINYTKAGGGCTSCHEKIELALAEILAQQPQTTPAVASGKDPHWQSVVDTIAELRPHIQADGGDMALLSVTNHQVTVSLSGSCSGCMMTDMTLAWLQQKLMERTGCYMEVVAAMWNYSEKVKDHFFNPRNARVVDNANAVGDVGSLSCGDALRLMLRVDPQSEIIEEAGFQTFGCGSAIASSSALTELIIGHTLAEAGQITNQQIADYLDGLPPEKMHCSVMGQEALRAAIANFRGESLEEEHDEGKLICKCFGVDEGHIRRAVQNNGLTTLAEVINYTKAGGGCTSCHEKIELALAEILAQQPQTTPAVASGKDPHWQSVVDTIAELRPHIQADGGDMALLSVTNHQVTVSLSGSCSGCMMTDMTLAWLQQKLMERTGCYMEVVAA

SEQ ID NO: 13 - protein, 381 aa, Klebsiella oxytoca
MERVLINDTTLRDGEQSPGVAFRTSEKVAIAEALYAAGITAMEVGTPAMGDEEIARIQLVRRQLPDATLMTWCRMNALEIRQSADLGIDWVDISIPASDKLRQYKLREPLAVLLERLAMFIHLAHTLGLKVCIGCEDASRASGQTLRAIAEVAQNAPAARLRYADTVGLLDPFTTAAQISALRDVWSGEIEMHAHNDLGMATANTLAAVSAGATSVNTTVLGLGERAGNAAAWKPSALGLERCLGVETGVHFSALPALCQRVAEAAQRAIDPQQPLVGELVFTHESGVHVAALLRDSESYQSIAPSLMGRSYRLVLGKHSGRQAVNGVFDQMGYHLNAAQINQLLPAIRRFAENWKRSPKDYELVAIYDELCGESALRARG

SEQ ID NO: 14 - protein, 156 aa, Klebsiella oxytoca
MPPINRQFDMVHSDEWSMKVAFASSDYRHVDQHFGATPRLVVYGVKADRVTLIRVVDFSVENGHQTEKIARRIHALEDCVTLFCVAIGDAVFRQLLQVGVRAERVPADTTIVGLLQEIQLYWYDKGQRKNQRQRDPERFTRLLQEQEWHGDPDPRR

SEQ ID NO: 15 - protein, 220 aa, Klebsiella oxytoca
MSDNDTLFWRMLALFQSLPDLQPAQIVDWLAQESGETLTPERLATLTQPQLAASFPSATAVMSPARWSRVMASLQGALPAHLRIVRPAQRTPQLLAAFCSQDGLVINGHFGQGRLFFIYAFDEQGGWLYDLRRYPSAPHQQEANEVRARLIEDCQLLFCQEIGGPAAARPIRHRIHPMKAQPGTTIQAQCEAINTLLAGRLPPWLAKRLNRDNPLEERVF

SEQ ID NO: 16 - protein, 148 aa, Klebsiella oxytoca
MRPKFTFSEEVRVVRAIRNDGTVAGFAPGALLVRRGSTGFVRDWGVFLQDQIIYQIHFPETDRIIGCREQELIPITQPWLAGNLQYRDSVTCQMALAVNGDVVVSAGQRGRVEATDRGELGDSYTVDFSGRWFRVPVQAIALIEEREE

SEQ ID NO: 17 - protein, 88 aa, Klebsiella oxytoca
MMEWFYQIPGVDELRSAESFFQFFAVPYQPELLGRCSLPVLATFHRKLRAEVPLQNRLEDNDRAPWLLARRLLAESYQQQFQESGTGG

SEQ ID NO: 18 - protein, 483 aa, Klebsiella oxytoca
MMTNATGERNLALIQEVLEVFPETARKERRKHMMVSDPEMESVGKCIISNRKSQPGVMTVRGCAYAGSKGVVFGPIKDMAHISHGPVGCGQYSRAGRRNYYTGVSGVDSFGTLNFTSDFQERDIVFGGDKKLSKLIEEMELLFPLTKGITIQSECPVGLIGDDISAVANASSKALDKPVIPVRCEGFRGVSQSLGHHIANDVVRDWILNNREGQPFETTPYDVAIIGDYNIGGDAWASRILLEEMGLRVVAQWSGDGTLVEMENTPFVKLNLVHCYRSMNYIARHMEEKHQIPWMEYNFFGPTKIAESLRKIADQFDDTIRANAEAVIARYEGQMAAIIAKYRPRLEGRKVLLYMGGLRPRHVIGAYEDLGMEIIAAGYEFAHNDDYDRTLPDLKEGTLLFDDASSYELEAFVKALKPDLIGSGIKEKYIFQKMGVPFRQMHSWDYSGPYHGYDGFAIFARDMDMTLNNPAWNELTAPWLKSA

SEQ ID NO: 19 - protein, 400 aa, Klebsiella oxytoca
MKQVYLDNNATTRLDPMVLEAMMPFLTDFYGNPSSIHDFGIPAQAALERAHQQAAALLGAEYPSEIIFTSCATEATATAIASAIALLPERREIITSVVEHPATLAACEHLERQGYRIHRIAVDSEGALDMAQFRAALSPRVALVSVMWANNETGVLFPIGEMAELAHEQGALFHCDAVQVVGKIPIAVGQTRIDMLSCSAHKFHGPKGVGCLYLRRGTRFRPLLRGGHQEYGRRAGTENICGIVGMGAACELANIHLPGMTHIGQLRNRLEHRLLASVPSVMVMGGGQPRVPGTVNLAFEFIEGEAILLLLNQAGIAASSGSACTSGSLEPSHVMRAMNIPYTAAHGTIRFSLSRYTREKEIDYVVATLPPIIDRLRALSPYWQNGKPRPADAVFTPVYG

SEQ ID NO: 20 - protein, 80 aa, Artificial Sequence
  Amino acid sequence of the N-terminal extension comprising the pFA  MTP (amino acids 1-77) and the amino acid triplet GAP (78-80). Cleavage by MPP occurs between amino acid residues 42 and 43
MAMAVFRREGRRLLPSIAARPIAAIRSPLSSDQEEGLLGVRSISTQVVRNRMKSVKNIQKITKAMKMVAASKLRAVQGAP

SEQ ID NO: 21 - protein, 54 aa, Artificial Sequence
  Amino acid sequence of the MTP-FA?51 polypeptide with additional N-terminal Met and C-terminal GG. Cleavage by MPP occurs between amino acid residues 43 and 44.
MMAMAVFRREGRRLLPSIAARPIAAIRSPLSSDQEEGLLGVRSISTQVVRNRGG

SEQ ID NO: 22 - protein, 9 aa, Artificial Sequence
  Amino acid sequence of the FAv-scar9 polypeptide.
ISTQVVRNR

SEQ ID NO: 23 - protein, 389 aa, Artificial Sequence
  Amino acid sequence of the MTP-FA 77::NifH::HA fusion polypeptide encoded by pRA10. Amino acids 1-77 correspond to MTP-FA 77, amino acids 78-80 are the GAP, amino acids 81-372 were the K. oxytoca NifH amino acids) and amino acids 373-389 include the HA
MAMAVFRREGRRLLPSIAARPIAAIRSPLSSDQEEGLLGVRSISTQVVRNRMKSVKNIQKITKAMKMVAASKLRAVQGAPTMRQCAIYGKGGIGKSTTTQNLVAALAEMGKKVMIVGCDPKADSTRLILHAKAQNTIMEMAAEVGSVEDLELEDVLQIGYGDVRCAESGGPEPGVGCAGRGVITAINFLEEEGAYEDDLDFVFYDVLGDVVCGGFAMPIRENKAQEIYIVCSGEMMAMYAANNISKGIVKYAKSGKVRLGGLICNSRQTDREDELIIALAEKLGTQMIHFVPRDNIVQRAEIRRMTVIEYDPACKQANEYRTLAQKIVNNTMKVVPTPCTMDELESLLMEFGIMEEEDTSIIGKTAAEENAAAGGGGGYPYDVPDYAPG

SEQ ID NO: 24 - protein, 363 aa, Artificial Sequence
  Amino acid sequence of the MTP-FA 51::NifH::HA fusion polypeptide encoded by pRA34. Amino acids 1-51 correspond to MTP-FA 51, amino acids 52-54 are the GAP, amino acids 55-346 were the K. oxytoca NifH amino acids and amino acids 347-363 include the HA
MAMAVFRREGRRLLPSIAARPIAAIRSPLSSDQEEGLLGVRSISTQVVRNRGAPTMRQCAIYGKGGIGKSTTTQNLVAALAEMGKKVMIVGCDPKADSTRLILHAKAQNTIMEMAAEVGSVEDLELEDVLQIGYGDVRCAESGGPEPGVGCAGRGVITAINFLEEEGAYEDDLDFVFYDVLGDVVCGGFAMPIRENKAQEIYIVCSGEMMAMYAANNISKGIVKYAKSGKVRLGGLICNSRQTDREDELIIALAEKLGTQMIHFVPRDNIVQRAEIRRMTVIEYDPACKQANEYRTLAQKIVNNTMKVVPTPCTMDELESLLMEFGIMEEEDTSIIGKTAAEENAAAGGGGGYPYDVPDYAPG

SEQ ID NO: 25 - protein, 358 aa, Artificial Sequence
  Amino acid sequence of the MTP-FAv51::NifH::HA fusion polypeptide encoded by SN18. Amino acids 1-54 correspond to the MTP-FA 51 with GG, amino acids 55-347 were the K. oxytoca NifH amino acids and amino acids 348-358 include the HA epitope
MMAMAVFRREGRRLLPSIAARPIAAIRSPLSSDQEEGLLGVRSISTQVVRNRGGMTMRQCAIYGKGGIGKSTTTQNLVAALAEMGKKVMIVGCDPKADSTRLILHAKAQNTIMEMAAEVGSVEDLELEDVLQIGYGDVRCAESGGPEPGVGCAGRGVITAINFLEEEGAYEDDLDFVFYDVLGDVVCGGFAMPIRENKAQEIYIVCSGEMMAMYAANNISKGIVKYAKSGKVRLGGLICNSRQTDREDELIIALAEKLGTQMIHFVPRDNIVQRAEIRRMTVIEYDPACKQANEYRTLAQKIVNNTMKVVPTPCTMDELESLLMEFGIMEEEDTSIIGKTAAEENAAGGYPYDVPDYA

SEQ ID NO: 26 - protein, 371 aa, Artificial Sequence
  Amino acid sequence of the MTP-FAv51::HA::NifH fusion polypeptide encoded by SN29. Amino acids 1-53 correspond to the MTP-FA 51 with GG, amino acids 54-64 include the HA epitope, amino acids 65-357 correspond to K. oxytoca NifH  and amino acids 358-371
MAMAVFRREGRRLLPSIAARPIAAIRSPLSSDQEEGLLGVRSISTQVVRNRGGYPYDVPDYAGGMTMRQCAIYGKGGIGKSTTTQNLVAALAEMGKKVMIVGCDPKADSTRLILHAKAQNTIMEMAAEVGSVEDLELEDVLQIGYGDVRCAESGGPEPGVGCAGRGVITAINFLEEEGAYEDDLDFVFYDVLGDVVCGGFAMPIRENKAQEIYIVCSGEMMAMYAANNISKGIVKYAKSGKVRLGGLICNSRQTDREDELIIALAEKLGTQMIHFVPRDNIVQRAEIRRMTVIEYDPACKQANEYRTLAQKIVNNTMKVVPTPCTMDELESLLMEFGIMEEEDTSIIGKTAAEENAAASLARVDRQARVSP

SEQ ID NO: 27 - protein, 9 aa, Artificial Sequence
  6xHis sequence used instead of the MTP, with N-terminal Met and C-terminal GG
MHHHHHHGG

SEQ ID NO: 28 - protein, 33 aa, Artificial Sequence
  Amino acid sequence of the CPN60 MTP
MYRFASNLASKARIAQNARQVSSRMSWSRNYGG

SEQ ID NO: 29 - protein, 31 aa, Artificial Sequence
  Amino acid sequence of the CPN60/No GGlinker MTP
MYRFASNLASKARIAQNARQVSSRMSWSRNY

SEQ ID NO: 30 - protein, 31 aa, Artificial Sequence
  Amino acid sequence of the Superoxide dismutase (SOD) MTP
MAIRCVASRKTLAGLKETSSRLLRIRGIQGG

SEQ ID NO: 31 - protein, 60 aa, Artificial Sequence
  Amino acid sequence of the Superoxide dismutase doubled (2SOD) MTP
MAIRCVASRKTLAGLKETSSRLLRIRGIQMAIRCVASRKTLAGLKETSSRLLRIRGIQGG

SEQ ID NO: 32 - protein, 29 aa, Artificial Sequence
  Amino acid sequence of the Superoxide dismutase, modified (SODmod) MTP
MAIRCVASRKTLAGLKETSSRLLRIRGGG

SEQ ID NO: 33 - protein, 56 aa, Artificial Sequence
  Amino acid sequence of the Superoxide dismutase, modified (2SODmod) doubled MTP
MAIRCVASRKTLAGLKETSSRLLRIRGMAIRCVASRKTLAGLKETSSRLLRIRGGG

SEQ ID NO: 34 - protein, 34 aa, Artificial Sequence
  Amino acid sequence of the L29 MTP (At1G07830)
MFLTRFVGRRFLAAASARSESTTAAAAASTIRGG

SEQ ID NO: 35 - protein, 70 aa, Neurospora crassa
MASTRVLASRLASQMAASAKVARPAVRVAQVSKRTIQTGSPLQTLKRTQMTSIVNATTRQAFQKRAYSGG

SEQ ID NO: 36 - protein, 53 aa, Artificial Sequence
  Amino acid sequence of the gATPase gamma subunit (FAv51) MTP, without the additional N-terminal Met. SEQ ID NO21 has an additional N-terminal Met. Cleavage by MPP occurs between amino acid residues 42 and 43.
MAMAVFRREGRRLLPSIAARPIAAIRSPLSSDQEEGLLGVRSISTQVVRNRGG

SEQ ID NO: 37 - protein, 61 aa, Artificial Sequence
  Amino acid sequence of the CoxIV twin strep (ABM97483) MTP
MLSLRQSIRFFKPATRTLCSSRYLLQQKPSAWSHPQFEKGGGSGGGSGGSAWSHPQFEKGG

SEQ ID NO: 38 - protein, 43 aa, Artificial Sequence
  Amino acid sequence of the CoxIV 10xHis (ABM97483) MTP
MLSLRQSIRFFKPATRTLCSSRYLLQQKPGGHHHHHHHHHHGG

SEQ ID NO: 39 - protein, 4 aa, Artificial Sequence
  Amino acid sequence of the predicted scar for the Superoxide dismutase (SOD) MTP with GG
IQGG

SEQ ID NO: 40 - protein, 15 aa, Artificial Sequence
  Amino acid sequence of the predicted scar for Superoxide dismutase, doubled (2SOD) MTP with GG
ESTTAAAAASTIRGG

SEQ ID NO: 41 - protein, 4 aa, Neurospora crassa
YSGG

SEQ ID NO: 42 - protein, 11 aa, Artificial Sequence
  Amino acid sequence of the predicted scar for the gATPase gamma subunit (FA?51) MTP with GG
ISTQVVRNRGG

SEQ ID NO: 43 - protein, 36 aa, Artificial Sequence
  Amino acid sequence of the predicted scar for the CoxIV twin strep MTP with GG
QQKPSAWSHPQFEKGGGSGGGSGGSAWSHPQFEKGG

SEQ ID NO: 44 - protein, 18 aa, Artificial Sequence
  Amino acid sequence of the predicted scar for the CoxIV 10xHis MTP with GG
QQKPGGHHHHHHHHHHGG

SEQ ID NO: 45 - DNA, 40 bp, Artificial Sequence
  Oligonucleotide primer MIT_V2.1_SbfInifH_FW2
aacctgcaggtgacgtctaagaaaaggaatattcagcaat

SEQ ID NO: 46 - DNA, 39 bp, Artificial Sequence
  Oligonucleotide primer MIT_V2.1_SbfInifJ_RV2
aacctgcagggctaactaactaaccacggacaaaaaacc

SEQ ID NO: 47 - DNA, 35 bp, Artificial Sequence
  Oligonucleotide primer MIT_V2.1_SbfInifB_FW
aacctgcaggtactctaaccccatcggccgtctta

SEQ ID NO: 48 - DNA, 35 bp, Artificial Sequence
  Oligonucleotide primer MIT_V2.1_SbfIori_RV
aacctgcaggtacgtagcaatcaactcactggctc

SEQ ID NO: 49 - protein, 9 aa, Artificial Sequence
  Amino acid sequence of mscar9 from MTP-FAv51 having substitution of the N-terminal Ile residue with a Met for translation initiation
MSTQVVRNR

SEQ ID NO: 50 - protein, 6 aa, Artificial Sequence
  Tryptic peptide
STQVVR

SEQ ID NO: 51 - protein, 9 aa, Artificial Sequence
  Amino acid sequence of MTP-FAv9 scar without N-terminal Met and with C-terminal Met
STQVVRNRM

SEQ ID NO: 52 - DNA, 47 bp, Artificial Sequence
  Oligonucleotide primer
atgtcaactcaagtggtgcgtaaccgcatgacctcttgttcgtcgtt

SEQ ID NO: 53 - DNA, 40 bp, Artificial Sequence
  Oligonucleotide primer
tttagccctcctatgattgatttgatgtattacagagagg

SEQ ID NO: 54 - DNA, 52 bp, Artificial Sequence
  Oligonucleotide primer
ggttacgcaccacttgagttgacattttagccctcctatgattgatttgatg

SEQ ID NO: 55 - protein, 7 aa, Artificial Sequence
  Tryptic peptide
MSTQVVR

SEQ ID NO: 56 - protein, 8 aa, Artificial Sequence
  Tryptic peptide
STQVVRNR

SEQ ID NO: 57 - protein, 599 aa, Artificial Sequence
  Amino acid sequence of the MTP-FA 77::NifK fusion polypeptide (pRA25), lacking any C-terminal extension. Amino acids 1-77 correspond to the MTP-FA 77, amino acids 78-80 are GAP, and amino acids 81-599 correspond to K. oxytoca NifK without the initiator
MAMAVFRREGRRLLPSIAARPIAAIRSPLSSDQEEGLLGVRSISTQVVRNRMKSVKNIQKITKAMKMVAASKLRAVQGAPSQTIDKINSCYPLFEQDEYQELFRNKRQLEEAHDAQRVQEVFAWTTTAEYEALNFRREALTVDPAKACQPLGAVLCSLGFANTLPYVHGSQGCVAYFRTYFNRHFKEPIACVSDSMTEDAAVFGGNNNMNLGLQNASALYKPEIIAVSTTCMAEVIGDDLQAFIANAKKDGFVDSSIAVPHAHTPSFIGSHVTGWDNMFEGFAKTFTADYQGQPGKLPKLNLVTGFETYLGNFRVLKRMMEQMAVPCSLLSDPSEVLDTPADGHYRMYSGGTTQQEMKEAPDAIDTLLLQPWQLLKSKKVVQEMWNQPATEVAIPLGLAATDELLMTVSQLSGKPIADALTLERGRLVDMMLDSHTWLHGKKFGLYGDPDFVMGLTRFLLELGCEPTVILSHNANKRWQKAMNKMLDASPYGRDSEVFINCDLWHFRSLMFTRQPDFMIGNSYGKFIQRDTLAKGKAFEVPLIRLGFPLFDRHHLHRQTTWGYEGAMNIVTTLVNAVLEKLDSDTSQLGKTDYSFDLVR

SEQ ID NO: 58 - protein, 4 aa, Klebsiella oxytoca
DLVR

SEQ ID NO: 59 - protein, 60 aa, Artificial Sequence
  Amino acid sequence of the mutant MTP-FAv51 polypeptide
MMAMAVFRREGRRLLPSIAARPIAAAAAAASSDQEEGLLAAAAAAAAVVRNRGGMMTNAT

SEQ ID NO: 60 - protein, 6 aa, Artificial Sequence
  peptide
VRGCAY

SEQ ID NO: 61 - protein, 10 aa, Artificial Sequence
  peptide
RAGRRNYYTG

SEQ ID NO: 62 - protein, 5 aa, Artificial Sequence
  peptide
SNRKS

SEQ ID NO: 63 - protein, 5 aa, Artificial Sequence
  peptide
AAAAA

SEQ ID NO: 64 - protein, 5 aa, Artificial Sequence
  peptide
QPGVM

SEQ ID NO: 65 - protein, 5 aa, Artificial Sequence
  peptide
TVRGC

SEQ ID NO: 66 - protein, 5 aa, Artificial Sequence
  peptide
AYAGS

SEQ ID NO: 67 - protein, 5 aa, Artificial Sequence
  peptide
GAGAA

SEQ ID NO: 68 - protein, 5 aa, Artificial Sequence
  peptide
KGVVF

SEQ ID NO: 69 - protein, 5 aa, Artificial Sequence
  peptide
GPIKD

SEQ ID NO: 70 - protein, 10 aa, Artificial Sequence
  peptide
TVRGCAYAGS

SEQ ID NO: 71 - protein, 10 aa, Artificial Sequence
  peptide
TARACGYGGS

SEQ ID NO: 72 - protein, 4 aa, Artificial Sequence
  peptide
AYAG

SEQ ID NO: 73 - protein, 4 aa, Artificial Sequence
  peptide
GAGG

SEQ ID NO: 74 - protein, 5 aa, Artificial Sequence
  peptide
MAHIS

SEQ ID NO: 75 - protein, 5 aa, Artificial Sequence
  peptide
AGAAA

SEQ ID NO: 76 - protein, 5 aa, Artificial Sequence
  peptide
HGPVG

SEQ ID NO: 77 - protein, 5 aa, Artificial Sequence
  peptide
CGQYS

SEQ ID NO: 78 - protein, 5 aa, Artificial Sequence
  peptide
RAGRR

SEQ ID NO: 79 - protein, 5 aa, Artificial Sequence
  peptide
NYYTG

SEQ ID NO: 80 - protein, 5 aa, Artificial Sequence
  peptide
VSGVD

SEQ ID NO: 81 - protein, 10 aa, Artificial Sequence
  peptide
RAGRRNYFTG

SEQ ID NO: 82 - protein, 10 aa, Artificial Sequence
  peptide
RAGRRNYYAG

SEQ ID NO: 83 - protein, 10 aa, Artificial Sequence
  peptide
RAGRRNYFAG

SEQ ID NO: 84 - protein, 10 aa, Artificial Sequence
  peptide
RAGRRNYYAA

SEQ ID NO: 85 - protein, 10 aa, Artificial Sequence
  peptide
RAGRANYYTG

SEQ ID NO: 86 - protein, 10 aa, Artificial Sequence
  peptide
RAGRRHYYTG

SEQ ID NO: 87 - protein, 10 aa, Artificial Sequence
  peptide
RAGRRNQYTG

SEQ ID NO: 88 - protein, 10 aa, Artificial Sequence
  peptide
RAGRRNYTTG

SEQ ID NO: 89 - protein, 10 aa, Artificial Sequence
  peptide
RAGRRNYYVG

SEQ ID NO: 90 - protein, 10 aa, Artificial Sequence
  peptide
RAGRRNQTTG

SEQ ID NO: 91 - protein, 10 aa, Artificial Sequence
  peptide
RAGRRHKGTG

SEQ ID NO: 92 - protein, 10 aa, Artificial Sequence
  peptide
RAGRRNKYTG

SEQ ID NO: 93 - protein, 10 aa, Artificial Sequence
  peptide
RAGRRNKATG

SEQ ID NO: 94 - protein, 10 aa, Artificial Sequence
  peptide
RAGRRNYATG

SEQ ID NO: 95 - protein, 10 aa, Artificial Sequence
  peptide
RAGRKNYYTG

SEQ ID NO: 96 - protein, 10 aa, Artificial Sequence
  peptide
RAGRKNYFTG

SEQ ID NO: 97 - protein, 10 aa, Artificial Sequence
  peptide
RAGRKNYYAG

SEQ ID NO: 98 - protein, 10 aa, Artificial Sequence
  peptide
RAGRKNYFAG

SEQ ID NO: 99 - protein, 10 aa, Artificial Sequence
  peptide
RAGRKNYAAG

SEQ ID NO: 100 - protein, 23 aa, Artificial Sequence
  peptide
YYTGVSGVDSFGTLNFTSDFQER

SEQ ID NO: 101 - protein, 4 aa, Artificial Sequence
  peptide
RRNY

SEQ ID NO: 102 - protein, 5 aa, Artificial Sequence
  peptide
RRNYY

SEQ ID NO: 103 - protein, 4 aa, Artificial Sequence
  peptide
RRNQ

SEQ ID NO: 104 - protein, 4 aa, Artificial Sequence
  peptide
RRNK

SEQ ID NO: 105 - protein, 5 aa, Artificial Sequence
  peptide
RRNQT

SEQ ID NO: 106 - protein, 4 aa, Artificial Sequence
  peptide
RRFK

SEQ ID NO: 107 - protein, 5 aa, Artificial Sequence
  peptide
RRNKA

SEQ ID NO: 108 - DNA, 56 bp, Artificial Sequence
  Oligonucleotide primer
ctaatgctaccggtgaacgtaacctggcactgattcaagaagtactggaagtgttc

SEQ ID NO: 109 - DNA, 57 bp, Artificial Sequence
  Oligonucleotide primer
gttacgttcaccggtagcattagtcatcatccggctcctccgctagataaaaatgtg

SEQ ID NO: 110 - DNA, 55 bp, Artificial Sequence
  Oligonucleotide primer
gtttctggcgtcgactctttcggcacgctgaacttcacctctgacttccaggaac

SEQ ID NO: 111 - DNA, 52 bp, Artificial Sequence
  Oligonucleotide primer
cgaaagagtcgacgccagaaacgcccgtgtagtagttacgacgtcccgcgcg

SEQ ID NO: 112 - DNA, 26 bp, Artificial Sequence
  Oligonucleotide primer
gaccaatgctaccggtgagaggaacc

SEQ ID NO: 113 - DNA, 29 bp, Artificial Sequence
  Oligonucleotide primer
gttaagagtcccgaaagagtcgacaccag

SEQ ID NO: 114 - protein, 11 aa, Artificial Sequence
  Amino acid sequence of an 11-residue section from a known unstructured linker region from Hypocrea jecorina cellobiohydrolase II (Accession no. AAG39980.1).
ATPPPGSTTTR

SEQ ID NO: 115 - protein, 9 aa, Artificial Sequence
  Amino acid sequence of 9-residue HA epitope
YPYDVPDYA

SEQ ID NO: 116 - protein, 30 aa, Artificial Sequence
  Amino acid sequence of linker for the NifD::linker::NifK fusion polypeptide.
ATPPPGSTTTAYPYDVPDYATPPPGSTTTA

SEQ ID NO: 117 - DNA, 47 bp, Artificial Sequence
  Oligonucleotide primer
gtcgtaaccaatacacgggcgtttctggcgtcgactctttcggcacg

SEQ ID NO: 118 - DNA, 41 bp, Artificial Sequence
  Oligonucleotide primer
gcccgtgtattggttacgacgtcccgcgcgagagtactggc

SEQ ID NO: 119 - protein, 7 aa, Artificial Sequence
  peptide sequence
ISTQVVR

SEQ ID NO: 120 - protein, 8 aa, Artificial Sequence
  peptide sequence
SISTQVVR

SEQ ID NO: 121 - protein, 603 aa, Artificial Sequence
  Amino acid sequence of the metaxin fusion polypeptide encoded by construct SN197. The TwinStrep epitope corresponds to amino acids 1-31, mTurquoise to amino acids 32-273, a TEV cleavage site to amino acids 274-282 and the metaxin sequence to amino acids
MWSHPQFEKGGGSGGGSGGSAWSHPQFEKGMVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYKGGGGENLYFQGGGMEEAKEREKLTLVTRKSSFGLPTSCPNCLPVYLYLKFSKTPFDLAFNLINPDFGQIPYVESGTYVAYNNEKGGVIRSLIEDGFVDLDSQVHGIPEWVSTKAMVDSWLADAILYELWVGSDGSSAHKIYFSDLPWPLGKLLYLKQVHVAKQILDITKDNAERREEEIYRNANDAFSALSTRLGEQAYLFDNRPTSLDAVFLGHALFTLYALPENSVLRNKLLEHDNLVRYTEKHKLELVDSSASSSSGTQSQSDPSSVPRRPSQWSSKPKSKPKREKTEEEKKFRRRAKYFLVTQLVAVLVFLSLLGGSGDAEVELDEDDYE

SEQ ID NO: 122 - protein, 547 aa, Artificial Sequence
  Amino acid sequence of the MTP-FA 51::NifD::HA fusion polypeptide encoded by SN10.  Amino acids 1-54 correspond to the MTP-FA 51 with GG at its C-terminus, amino acids 55-536 were the K. oxytoca NifD amino acids (SEQ ID NO18) with its initiator Met, and amino
MMAMAVFRREGRRLLPSIAARPIAAIRSPLSSDQEEGLLGVRSISTQVVRNRGGMMTNATGERNLALIQEVLEVFPETARKERRKHMMVSDPEMESVGKCIISNRKSQPGVMTVRGCAYAGSKGVVFGPIKDMAHISHGPVGCGQYSRAGRRNYYTGVSGVDSFGTLNFTSDFQERDIVFGGDKKLSKLIEEMELLFPLTKGITIQSECPVGLIGDDISAVANASSKALDKPVIPVRCEGFRGVSQSLGHHIANDVVRDWILNNREGQPFETTPYDVAIIGDYNIGGDAWASRILLEEMGLRVVAQWSGDGTLVEMENTPFVKLNLVHCYRSMNYIARHMEEKHQIPWMEYNFFGPTKIAESLRKIADQFDDTIRANAEAVIARYEGQMAAIIAKYRPRLEGRKVLLYMGGLRPRHVIGAYEDLGMEIIAAGYEFAHNDDYDRTLPDLKEGTLLFDDASSYELEAFVKALKPDLIGSGIKEKYIFQKMGVPFRQMHSWDYSGPYHGYDGFAIFARDMDMTLNNPAWNELTAPWLKSGGYPYDVPDYA

SEQ ID NO: 123 - protein, 331 aa, Artificial Sequence
  Amino acid sequence of the MTP-FA 51::NifM::HA fusion polypeptide encoded by SN30. Amino acids 1-54 correspond to the MTP-FA 51 with GG at its C-terminus, amino acids 55-320 correspond to K. oxytoca NifM (SEQ ID NO8) with its initiator Met, and amino
MMAMAVFRREGRRLLPSIAARPIAAIRSPLSSDQEEGLLGVRSISTQVVRNRGGMNPWQRFARQRLARSRWNRDPAALDPADTPAFEQAWQRQCHMEQTIVARVPEGDIPAALLENIAASLAIWLDEGDFAPPERAAIVRHHARLELAFADIARQAPQPDLSTVQAWYLRHQTQFMRPEQRLTRHLLLTVDNDREAVHQRILGLYRQINASRDAFAPLAQRHSHCPSALEEGRLGWISRGLLYPQLETALFSLAENALSLPIASELGWHLLWCEAIRPAAPMEPQQALESARDYLWQQSQQRHQRQWLEQMISRQPGLCGGGYPYDVPDYA

SEQ ID NO: 124 - protein, 465 aa, Artificial Sequence
  Amino acid sequence of the MTP-FA 51::NifS::HA fusion polypeptide encoded by SN31. Amino acids 1-54 correspond to the MTP-FA 51 with GG at its C-terminus, amino acids 55-454 correspond to K. oxytoca NifS (SEQ ID NO19) with its initiator Met, according to
MMAMAVFRREGRRLLPSIAARPIAAIRSPLSSDQEEGLLGVRSISTQVVRNRGGMKQVYLDNNATTRLDPMVLEAMMPFLTDFYGNPSSIHDFGIPAQAALERAHQQAAALLGAEYPSEIIFTSCATEATATAIASAIALLPERREIITSVVEHPATLAACEHLERQGYRIHRIAVDSEGALDMAQFRAALSPRVALVSVMWANNETGVLFPIGEMAELAHEQGALFHCDAVQVVGKIPIAVGQTRIDMLSCSAHKFHGPKGVGCLYLRRGTRFRPLLRGGHQEYGRRAGTENICGIVGMGAACELANIHLPGMTHIGQLRNRLEHRLLASVPSVMVMGGGQPRVPGTVNLAFEFIEGEAILLLLNQAGIAASSGSACTSGSLEPSHVMRAMNIPYTAAHGTIRFSLSRYTREKEIDYVVATLPPIIDRLRALSPYWQNGKPRPADAVFTPVYGGGYPYDVPDYA

SEQ ID NO: 125 - protein, 339 aa, Artificial Sequence
  Amino acid sequence of the MTP-FA 51::NifU::HA fusion polypeptide encoded by SN32. Amino acids 1-54 correspond to the MTP-FA 51 with GG at its C-terminus, amino acids 55-328 correspond to K. oxytoca NifU (SEQ ID NO12) with its initiator Met, and amino
MMAMAVFRREGRRLLPSIAARPIAAIRSPLSSDQEEGLLGVRSISTQVVRNRGGMWNYSEKVKDHFFNPRNARVVDNANAVGDVGSLSCGDALRLMLRVDPQSEIIEEAGFQTFGCGSAIASSSALTELIIGHTLAEAGQITNQQIADYLDGLPPEKMHCSVMGQEALRAAIANFRGESLEEEHDEGKLICKCFGVDEGHIRRAVQNNGLTTLAEVINYTKAGGGCTSCHEKIELALAEILAQQPQTTPAVASGKDPHWQSVVDTIAELRPHIQADGGDMALLSVTNHQVTVSLSGSCSGCMMTDMTLAWLQQKLMERTGCYMEVVAAGGYPYDVPDYA

SEQ ID NO: 126 - protein, 522 aa, Artificial Sequence
  Amino acid sequence of the MTP-FA 51::NifE::HA fusion polypeptide encoded by SN38. Amino acids 1-54 correspond to the MTP-FA 51 with GG at its C-terminus, amino acids 55-511 correspond to K. oxytoca NifE with its initiator Met according to Temme et al.,
MMAMAVFRREGRRLLPSIAARPIAAIRSPLSSDQEEGLLGVRSISTQVVRNRGGMKGNEILALLDEPACEHNHKQKSGCSAPKPGATAGGCAFDGAQITLLPIADVAHLVHGPIGCAGSSWDNRGSASSGPTLNRLGFTTDLNEQDVIMGRGERRLFHAVRHIVTRYHPAAVFIYNTCVPAMEGDDLEAVCQAAQTATGVPVIAIDAAGFYGSKNLGNRLAGDVMVKRVIGQREPAPWPESTLFAPEQRHDIGLIGEFNIAGEFWHIQPLLDELGIRVLGSLSGDGRFAEIQTMHRAQANMLVCSRALINVARALEQRYGTPWFEGSFYGIRATSDALRQLAALLGDDDLRQRTEALIAREEQAAELALQPWREQLRGRKALLYTGGVKSWSVVSALQDLGMTVVATGTRKSTEEDKQRIRELMGEEAVMLEEGNARTLLDVVYRYQADLMIAGGRNMYTAYKARLPFLDINQEREHAFAGYQGIVTLARQLCQTINSPIWPQTHSRAPWRGGYPYDVPDYA

SEQ ID NO: 127 - protein, 526 aa, Artificial Sequence
  Amino acid sequence of the MTP-FA 51::NifN::HA fusion polypeptide encoded by SN39. Amino acids 1-54 correspond to the MTP-FA 51 with GG at its C-terminus, amino acids 55-515 correspond to K. oxytoca NifN (SEQ ID NO9) with its initiator Met, and amino
MMAMAVFRREGRRLLPSIAARPIAAIRSPLSSDQEEGLLGVRSISTQVVRNRGGMADIFRTDKPLAVSPIKTGQPLGAILASLGIEHSIPLVHGAQGCSAFAKVFFIQHFHDPVPLQSTAMDPTSTIMGADGNIFTALDTLCQRNNPQAIVLLSTGLSEAQGSDISRVVRQFREEYPRHKGVAILTVNTPDFYGSMENGFSAVLESVIEQWVPPAPRPAQRNRRVNLLVSHLCSPGDIEWLRRCVEAFGLQPIILPDLAQSMDGHLAQGDFSPLTQGGTPLRQIEQMGQSLCSFAIGVSLHRASSLLAPRCRGEVIALPHLMTLERCDAFIHQLAKISGRAVPEWLERQRGQLQDAMIDCHMWLQGQRMAIAAEGDLLAAWCDFANSQGMQPGPLVAPTGHPSLRQLPVERVVPGDLEDLQTLLCAHPADLLVANSHARDLAEQFALPLVRAGFPLFDKLGEFRRVRQGYSGMRDTLFELANLIRERHHHLAHYRSPLRQNPESSLSTGGAYAADGGYPYDVPDYA

SEQ ID NO: 128 - protein, 365 aa, Artificial Sequence
  Amino acid sequence of the MTP-CoxIV-Twin-Strep::NifH::HA fusion polypeptide encoded by SN42. Amino acids 1-61 correspond to the MTP-CoxIV-Twin-Strep with GG at its C-terminus, amino acids 62-354 were the K. oxytoca NifH amino acids (SEQ ID NO1) with
MLSLRQSIRFFKPATRTLCSSRYLLQQKPSAWSHPQFEKGGGSGGGSGGSAWSHPQFEKGGMTMRQCAIYGKGGIGKSTTTQNLVAALAEMGKKVMIVGCDPKADSTRLILHAKAQNTIMEMAAEVGSVEDLELEDVLQIGYGDVRCAESGGPEPGVGCAGRGVITAINFLEEEGAYEDDLDFVFYDVLGDVVCGGFAMPIRENKAQEIYIVCSGEMMAMYAANNISKGIVKYAKSGKVRLGGLICNSRQTDREDELIIALAEKLGTQMIHFVPRDNIVQRAEIRRMTVIEYDPACKQANEYRTLAQKIVNNTMKVVPTPCTMDELESLLMEFGIMEEEDTSIIGKTAAEENAAGGYPYDVPDYA

SEQ ID NO: 129 - protein, 590 aa, Artificial Sequence
  Amino acid sequence of the MTP-Su9::NifK fusion polypeptide encoded by SN46. Amino acids 1-70 correspond to the MTP-Su9 with GG at its C-terminus, amino acids 71-590 correspond to K. oxytoca NifK (SEQ ID NO3) with its initiator Met.
MASTRVLASRLASQMAASAKVARPAVRVAQVSKRTIQTGSPLQTLKRTQMTSIVNATTRQAFQKRAYSGGMSQTIDKINSCYPLFEQDEYQELFRNKRQLEEAHDAQRVQEVFAWTTTAEYEALNFQREALTVDPAKACQPLGAVLCSLGFANTLPYVHGSQGCVAYFRTYFNRHFKEPIACVSDSMTEDAAVFGGNNNMNLGLQNASALYKPEIIAVSTTCMAEVIGDDLQAFIANAKKDGFVDSSIAVPHAHTPSFIGSHVTGWDNMFEGFAKTFTADYQGQPGKLPKLNLVTGFETYLGNFRVLKRMMEQMAVPCSLLSDPSEVLDTPADGHYRMYSGGTTQQEMKEAPDAIDTLLLQPWQLLKSKKVVQEMWNQPATEVAIPLGLAATDELLMTVSQLSGKPIADALTLERGRLVDMMLDSHTWLHGKKFGLYGDPDFVMGLTRFLLELGCEPTVILSHNANKRWQKAMNKMLDASPYGRDSEVFINCDLWHFRSLMFTRQPDFMIGNSYGKFIQRDTLAKGKAFEVPLIRLGFPLFDRHHLHRQTTWGYEGAMNIVTTLVNAVLEKLDSDTSQLGKTDYSFDLVR

SEQ ID NO: 130 - protein, 426 aa, Artificial Sequence
  Amino acid sequence of the MTP-L29::NifV::HA fusion polypeptide encoded by SN51. Amino acids 1-34 correspond to the MTP-L29 with GG at its C-terminus, amino acids 35-415 correspond to K. oxytoca NifV (SEQ ID NO13) with its initiator Met, and amino acids 416-
MFLTRFVGRRFLAAASARSESTTAAAAASTIRGGMERVLINDTTLRDGEQSPGVAFRTSEKVAIAEALYAAGITAMEVGTPAMGDEEIARIQLVRRQLPDATLMTWCRMNALEIRQSADLGIDWVDISIPASDKLRQYKLREPLAVLLERLAMFIHLAHTLGLKVCIGCEDASRASGQTLRAIAEVAQNAPAARLRYADTVGLLDPFTTAAQISALRDVWSGEIEMHAHNDLGMATANTLAAVSAGATSVNTTVLGLGERAGNAAAWKPSALGLERCLGVETGVHFSALPALCQRVAEAAQRAIDPQQPLVGELVFTHESGVHVAALLRDSESYQSIAPSLMGRSYRLVLGKHSGRQAVNGVFDQMGYHLNAAQINQLLPAIRRFAENWKRSPKDYELVAIYDELCGESALRARGGGYPYDVPDYA

SEQ ID NO: 131 - protein, 1085 aa, Artificial Sequence
  Amino acid sequence of the MTP-FA 51::NifD::linker(HA)::NifK fusion polypeptide encoded by SN68. Amino acids 1-54 correspond to the MTP-FA 51 with GG at its C-terminus, amino acids 55-536 correspond to wild-type K. oxytoca NifD amino acids (SEQ ID NO18
MMAMAVFRREGRRLLPSIAARPIAAIRSPLSSDQEEGLLGVRSISTQVVRNRGGMTNATGERNLALIQEVLEVFPETARKERRKHMMVSDPEMESVGKCIISNRKSQPGVMTVRGCAYAGSKGVVFGPIKDMAHISHGPVGCGQYSRAGRRNYYTGVSGVDSFGTLNFTSDFQERDIVFGGDKKLSKLIEEMELLFPLTKGITIQSECPVGLIGDDISAVANASSKALDKPVIPVRCEGFRGVSQSLGHHIANDVVRDWILNNREGQPFETTPYDVAIIGDYNIGGDAWASRILLEEMGLRVVAQWSGDGTLVEMENTPFVKLNLVHCYRSMNYIARHMEEKHQIPWMEYNFFGPTKIAESLRKIADQFDDTIRANAEAVIARYEGQMAAIIAKYRPRLEGRKVLLYMGGLRPRHVIGAYEDLGMEIIAAGYEFAHNDDYDRTLPDLKEGTLLFDDASSYELEAFVKALKPDLIGSGIKEKYIFQKMGVPFRQMHSWDYSGPYHGYDGFAIFARDMDMTLNNPAWNELTAPWLKSAATPPPGSTTTAYPYDVPDYATPPPGSTTTASQTIDKINSCYPLFEQDEYQELFRNKRQLEEAHDAQRVQEVFAWTTTAEYEALNFQREALTVDPAKACQPLGAVLCSLGFANTLPYVHGSQGCVAYFRTYFNRHFKEPIACVSDSMTEDAAVFGGNNNMNLGLQNASALYKPEIIAVSTTCMAEVIGDDLQAFIANAKKDGFVDSSIAVPHAHTPSFIGSHVTGWDNMFEGFAKTFTADYQGQPGKLPKLNLVTGFETYLGNFRVLKRMMEQMAVPCSLLSDPSEVLDTPADGHYRMYSGGTTQQEMKEAPDAIDTLLLQPWQLLKSKKVVQEMWNQPATEVAIPLGLAATDELLMTVSQLSGKPIADALTLERGRLVDMMLDSHTWLHGKKFGLYGDPDFVMGLTRFLLELGCEPTVILSHNANKRWQKAMNKMLDASPYGRDSEVFINCDLWHFRSLMFTRQPDFMIGNSYGKFIQRDTLAKGKAFEVPLIRLGFPLFDRHHLHRQTTWGYEGAMNIVTTLVNAVLEKLDSDTSQLGKTDYSFDLVR

SEQ ID NO: 132 - protein, 557 aa, Artificial Sequence
  Amino acid sequence of the MTP-FA 51::HA::NifD::HA fusion polypeptide encoded by SN75. Amino acids 1-53 correspond to the MTP-FA 51 with GG at its C-terminus, amino acids 54-64 correspond to the first HA epitope, amino acids 65-546 correspond to
MAMAVFRREGRRLLPSIAARPIAAIRSPLSSDQEEGLLGVRSISTQVVRNRGGYPYDVPDYAGGMMTNATGERNLALIQEVLEVFPETARKERRKHMMVSDPEMESVGKCIISNRKSQPGVMTVRGCAYAGSKGVVFGPIKDMAHISHGPVGCGQYSRAGRRNYYTGVSGVDSFGTLNFTSDFQERDIVFGGDKKLSKLIEEMELLFPLTKGITIQSECPVGLIGDDISAVANASSKALDKPVIPVRCEGFRGVSQSLGHHIANDVVRDWILNNREGQPFETTPYDVAIIGDYNIGGDAWASRILLEEMGLRVVAQWSGDGTLVEMENTPFVKLNLVHCYRSMNYIARHMEEKHQIPWMEYNFFGPTKIAESLRKIADQFDDTIRANAEAVIARYEGQMAAIIAKYRPRLEGRKVLLYMGGLRPRHVIGAYEDLGMEIIAAGYEFAHNDDYDRTLPDLKEGTLLFDDASSYELEAFVKALKPDLIGSGIKEKYIFQKMGVPFRQMHSWDYSGPYHGYDGFAIFARDMDMTLNNPAWNELTAPWLKSGGYPYDVPDYA

SEQ ID NO: 133 - protein, 547 aa, Artificial Sequence
  Amino acid sequence of the MTP-FA 51::NifD::HA fusion polypeptide encoded by SN99. Amino acids 1-54 correspond to the MTP-FA 51 with GG at its C-terminus, amino acids 55-536 correspond to K. oxytoca NifD comprising the alanine substitution mutations at
MMAMAVFRREGRRLLPSIAARPIAAIRSPLSSDQEEGLLGVRSISTQVVRNRGGMMTNATGERNLALIQEVLEVFPETARKERRKHMMVSDPEMESVGKCIISNRKSQPGVMTVRGCAYAGSKGVVFGPIKDMAHISHGPVGCGQYSAAAAANYYTGVSGVDSFGTLNFTSDFQERDIVFGGDKKLSKLIEEMELLFPLTKGITIQSECPVGLIGDDISAVANASSKALDKPVIPVRCEGFRGVSQSLGHHIANDVVRDWILNNREGQPFETTPYDVAIIGDYNIGGDAWASRILLEEMGLRVVAQWSGDGTLVEMENTPFVKLNLVHCYRSMNYIARHMEEKHQIPWMEYNFFGPTKIAESLRKIADQFDDTIRANAEAVIARYEGQMAAIIAKYRPRLEGRKVLLYMGGLRPRHVIGAYEDLGMEIIAAGYEFAHNDDYDRTLPDLKEGTLLFDDASSYELEAFVKALKPDLIGSGIKEKYIFQKMGVPFRQMHSWDYSGPYHGYDGFAIFARDMDMTLNNPAWNELTAPWLKSGGYPYDVPDYA

SEQ ID NO: 134 - protein, 547 aa, Artificial Sequence
  Amino acid sequence of the MTP-FA 51::NifD::HA fusion polypeptide encoded by SN100. Amino acids 1-54 correspond to the MTP-FA 51 with GG at its C-terminus, amino acids 55-536 correspond to K. oxytoca NifD amino acids comprising the alanine substitution
MMAMAVFRREGRRLLPSIAARPIAAIRSPLSSDQEEGLLGVRSISTQVVRNRGGMMTNATGERNLALIQEVLEVFPETARKERRKHMMVSDPEMESVGKCIISNRKSQPGVMTVRGCAYAGSKGVVFGPIKDMAHISHGPVGCGQYSRAGRRAAAAAVSGVDSFGTLNFTSDFQERDIVFGGDKKLSKLIEEMELLFPLTKGITIQSECPVGLIGDDISAVANASSKALDKPVIPVRCEGFRGVSQSLGHHIANDVVRDWILNNREGQPFETTPYDVAIIGDYNIGGDAWASRILLEEMGLRVVAQWSGDGTLVEMENTPFVKLNLVHCYRSMNYIARHMEEKHQIPWMEYNFFGPTKIAESLRKIADQFDDTIRANAEAVIARYEGQMAAIIAKYRPRLEGRKVLLYMGGLRPRHVIGAYEDLGMEIIAAGYEFAHNDDYDRTLPDLKEGTLLFDDASSYELEAFVKALKPDLIGSGIKEKYIFQKMGVPFRQMHSWDYSGPYHGYDGFAIFARDMDMTLNNPAWNELTAPWLKSGGYPYDVPDYA

SEQ ID NO: 135 - protein, 167 aa, Artificial Sequence
  Amino acid sequence of the MTP-Su9::NifW fusion polypeptide encoded by SN104. Amino acids 1-70 correspond to the MTP-Su9 with GG at its C-terminus, amino acids 71-158 correspond to K. oxytoca NifW (SEQ ID NO17) with its initiator Met, and amino
MASTRVLASRLASQMAASAKVARPAVRVAQVSKRTIQTGSPLQTLKRTQMTSIVNATTRQAFQKRAYSGGMMEWFYQIPGVDELRSAESFFQFFAVPYQPELLGRCSLPVLATFHRKLRAEVPLQNRLEDNDRAPWLLARRLLAESYQQQFQESGTGGYPYDVPDYA

SEQ ID NO: 136 - protein, 547 aa, Artificial Sequence
  Amino acid sequence of the MTP-FA 51::NifD::HA fusion polypeptide encoded by SN114. Amino acids 1-54 correspond to the MTP-FA 51 with GG at its C-terminus, amino acids 55-536 correspond to K. oxytoca NifD comprising the Y100Q substitution mutation at amino
MMAMAVFRREGRRLLPSIAARPIAAIRSPLSSDQEEGLLGVRSISTQVVRNRGGMMTNATGERNLALIQEVLEVFPETARKERRKHMMVSDPEMESVGKCIISNRKSQPGVMTVRGCAYAGSKGVVFGPIKDMAHISHGPVGCGQYSRAGRRNQYTGVSGVDSFGTLNFTSDFQERDIVFGGDKKLSKLIEEMELLFPLTKGITIQSECPVGLIGDDISAVANASSKALDKPVIPVRCEGFRGVSQSLGHHIANDVVRDWILNNREGQPFETTPYDVAIIGDYNIGGDAWASRILLEEMGLRVVAQWSGDGTLVEMENTPFVKLNLVHCYRSMNYIARHMEEKHQIPWMEYNFFGPTKIAESLRKIADQFDDTIRANAEAVIARYEGQMAAIIAKYRPRLEGRKVLLYMGGLRPRHVIGAYEDLGMEIIAAGYEFAHNDDYDRTLPDLKEGTLLFDDASSYELEAFVKALKPDLIGSGIKEKYIFQKMGVPFRQMHSWDYSGPYHGYDGFAIFARDMDMTLNNPAWNELTAPWLKSGGYPYDVPDYA

SEQ ID NO: 137 - protein, 241 aa, Artificial Sequence
  Amino acid sequence of the MTP-FA 51::NifF::HA fusion polypeptide encoded by SN138. Amino acids 1-54 correspond to the MTP-FA 51 with GG, amino acids 55-230 correspond to K. oxytoca NifF (SEQ ID NO6) and amino acids 231-241 include the HA epitope.
MMAMAVFRREGRRLLPSIAARPIAAIRSPLSSDQEEGLLGVRSISTQVVRNRGGMANIGIFFGTDTGKTRKIAKMIHKQLGELADAPVNINRTTLDDFMAYPVLLLGTPTLGDGQLPGLEAGCESESWSEFISGLDDASLKGKTVALFGLGDQRGYPDNFVSGMRPLFDALSARGAQMIGSWPNEGYEFSASSALEGDRFVGLVLDQDNQFDQTEARLASWLEEIKRTVLGGYPYDVPDYA

SEQ ID NO: 138 - protein, 1236 aa, Artificial Sequence
  Amino acid sequence of the MTP-FA 51::NifJ::HA fusion polypeptide encoded by SN139. Amino acids 1-54 correspond to the MTP-FA 51 with GG, amino acids 55-1225 correspond to K. oxytoca NifJ (SEQ ID NO7), and amino acids 1226-1236 include the HA epitope.
MMAMAVFRREGRRLLPSIAARPIAAIRSPLSSDQEEGLLGVRSISTQVVRNRGGMSGKMKTMDGNAAAAWISYAFTEVAAIYPITPSTPMAENVDEWAAQGKKNLFGQPVRLMEMQSEAGAAGAVHGALQAGALTTTYTASQGLLLMIPNMYKIAGELLPGVFHVSARALATNSLNIFGDHQDVMAVRQTGCAMLAENNVQQVMDLSAVAHLAAIKGRIPFVNFFDGFRTSHEIQKIEVLEYEQLATLLDRPALDSFRRNALHPDHPVIRGTAQNPDIYFQEREAGNRFYQALPDIVESYMTQISALTGREYHLFNYTGAADAERVIIAMGSVCDTVQEVVDTLNAAGEKVGLLSVHLFRPFSLAHFFAQLPKTVQRIAVLDRTKEPGAQAEPLCLDVKNAFYHHDDAPLIVGGRYALGGKDVLPNDIAAVFDNLNKPLPMDGFTLGIVDDVTFTSLPPRQQTLAVSHDGITACKFWGMGSDGTVGANKSAIKIIGDKTPLYAQAYFSYDSKKSGGITVSHLRFGDRPINSPYLIHRADFISCSQQSYVERYDLLDGLKPGGTFLLNCSWSDAELEQHLPVGFKRYLARENIHFYTLNAVDIARELGLGGRFNMLMQAAFFKLAAIIDPQTAADYLKQAVEKSYGSKGAAVIEMNQRAIELGMASLHQVTIPAHWATLDEPAAQASAMMPDFIRDILQPMNRQCGDQLPVSAFVGMEDGTFPSGTAAWEKRGIALEVPVWQPEGCTQCNQCAFICPHAAIRPALLNGEEHDAAPVGLLSKPAQGAKEYHYHLAISPLDCSGCGNCVDICPARGKALKMQSLDSQRQMAPVWDYALALTPKSNPFRKTTVKGSQFETPLLEFSGACAGCGETPYARLITQLFGDRMLIANATGCSSIWGASAPSIPYTTNHRGHGPAWANSLFEDNAEFGLGMMLGGQAVRQQIADDMTAALALPVSDELSDAMRQWLAKQDEGEGTRERADRLSERLAAEKEGVPLLEQLWQNRDYFVRRSQWIFGGDGWAYDIGFGGLDHVLASGEDVNILVFDTEVYSNTGGQSSKSTPVAAIAKFAAQGKRTRKKDLGMMAMSYGNVYVAQVAMGADKDQTLRAIAEAEAWPGPSLVIAYAACINHGLKAGMRCSQREAKRAVEAGYWHLWRYHPQREAEGKTPFMLDSEEPEESFRDFLLGEVRYASLHKTTPHLADALFSRTEEDARARFAQYRRLAGEEGGYPYDVPDYA

SEQ ID NO: 139 - protein, 584 aa, Artificial Sequence
  Amino acid sequence of the MTP-FA 51::HA::NifK fusion polypeptide encoded by SN140. Amino acids 1-53 correspond to the MTP-FA 51 with GG, amino acids 54-64 include the HA epitope, and amino acids 65-584 correspond to K. oxytoca NifK (SEQ ID NO3) with
MAMAVFRREGRRLLPSIAARPIAAIRSPLSSDQEEGLLGVRSISTQVVRNRGGYPYDVPDYAGGMSQTIDKINSCYPLFEQDEYQELFRNKRQLEEAHDAQRVQEVFAWTTTAEYEALNFQREALTVDPAKACQPLGAVLCSLGFANTLPYVHGSQGCVAYFRTYFNRHFKEPIACVSDSMTEDAAVFGGNNNMNLGLQNASALYKPEIIAVSTTCMAEVIGDDLQAFIANAKKDGFVDSSIAVPHAHTPSFIGSHVTGWDNMFEGFAKTFTADYQGQPGKLPKLNLVTGFETYLGNFRVLKRMMEQMAVPCSLLSDPSEVLDTPADGHYRMYSGGTTQQEMKEAPDAIDTLLLQPWQLLKSKKVVQEMWNQPATEVAIPLGLAATDELLMTVSQLSGKPIADALTLERGRLVDMMLDSHTWLHGKKFGLYGDPDFVMGLTRFLLELGCEPTVILSHNANKRWQKAMNKMLDASPYGRDSEVFINCDLWHFRSLMFTRQPDFMIGNSYGKFIQRDTLAKGKAFEVPLIRLGFPLFDRHHLHRQTTWGYEGAMNIVTTLVNAVLEKLDSDTSQLGKTDYSFDLVR

SEQ ID NO: 140 - protein, 232 aa, Artificial Sequence
  Amino acid sequence of the MTP-FA 51::NifQ::HA fusion polypeptide encoded by SN141. Amino acids 1-54 correspond to the MTP-FA 51 with GG, amino acids 55-221 correspond to K. oxytoca NifQ (SEQ ID NO10) and amino acids 222-232 include the HA epitope.
MMAMAVFRREGRRLLPSIAARPIAAIRSPLSSDQEEGLLGVRSISTQVVRNRGGMPPLDWLRRLWLLYHAGKGSFPLRMGLSPRDWQALRRRLGEVETPLDGETLTRRRLMAELNATREEERQQLGAWLAGWMQQDAGPMAQIIAEVSLAFNHLWQDLGLASRAELRLLMSDCFPQLVVMNEHNMRWKKFFYRQRCLLQQGEVICRSPSCDECWERSACFEGGYPYDVPDYA

SEQ ID NO: 141 - protein, 446 aa, Artificial Sequence
  Amino acid sequence of the MTP-FA 51::NifV::HA fusion polypeptide encoded by SN142. Amino acids 1-54 correspond to the MTP-FA 51 with GG, amino acids 55-435 correspond to K. oxytoca NifV (SEQ ID NO13) and amino acids 436-446 include the HA epitope.
MMAMAVFRREGRRLLPSIAARPIAAIRSPLSSDQEEGLLGVRSISTQVVRNRGGMERVLINDTTLRDGEQSPGVAFRTSEKVAIAEALYAAGITAMEVGTPAMGDEEIARIQLVRRQLPDATLMTWCRMNALEIRQSADLGIDWVDISIPASDKLRQYKLREPLAVLLERLAMFIHLAHTLGLKVCIGCEDASRASGQTLRAIAEVAQNAPAARLRYADTVGLLDPFTTAAQISALRDVWSGEIEMHAHNDLGMATANTLAAVSAGATSVNTTVLGLGERAGNAAAWKPSALGLERCLGVETGVHFSALPALCQRVAEAAQRAIDPQQPLVGELVFTHESGVHVAALLRDSESYQSIAPSLMGRSYRLVLGKHSGRQAVNGVFDQMGYHLNAAQINQLLPAIRRFAENWKRSPKDYELVAIYDELCGESALRARGGGYPYDVPDYA

SEQ ID NO: 142 - protein, 151 aa, Artificial Sequence
  Amino acid sequence of the MTP-FA 51::NifW::HA fusion polypeptide encoded by SN143. Amino acids 1-54 correspond to the MTP-FA 51 with GG, amino acids 55-140 correspond to K. oxytoca NifW (SEQ ID NO17), and amino acids 141-151 include the HA epitope.
MMAMAVFRREGRRLLPSIAARPIAAIRSPLSSDQEEGLLGVRSISTQVVRNRGGMMEWFYQIPGVDELRSAESFFQFFAVPYQPELLGRCSLPVLATFHRKLRAEVPLQNRLEDNDRAPWLLARRLLAESYQQQFQESGTGGYPYDVPDYA

SEQ ID NO: 143 - protein, 221 aa, Artificial Sequence
  Amino acid sequence of the MTP-FA 51::NifX::HA fusion polypeptide encoded by SN144. Amino acids 1-54 correspond to the MTP-FA 51 with GG, amino acids 55-210 correspond to K. oxytoca NifX (SEQ ID NO14), and amino acids 211-221 include the HA epitope.
MMAMAVFRREGRRLLPSIAARPIAAIRSPLSSDQEEGLLGVRSISTQVVRNRGGMPPINRQFDMVHSDEWSMKVAFASSDYRHVDQHFGATPRLVVYGVKADRVTLIRVVDFSVENGHQTEKIARRIHALEDCVTLFCVAIGDAVFRQLLQVGVRAERVPADTTIVGLLQEIQLYWYDKGQRKNQRQRDPERFTRLLQEQEWHGDPDPRRGGYPYDVPDYA

SEQ ID NO: 144 - protein, 285 aa, Artificial Sequence
  Amino acid sequence of the MTP-FA 51::NifY::HA fusion polypeptide encoded by SN145. Amino acids 1-54 correspond to the MTP-FA 51 with GG, amino acids 55-274 correspond to K. oxytoca NifY according to Temme et al., (2012), and amino acids 275-285
MMAMAVFRREGRRLLPSIAARPIAAIRSPLSSDQEEGLLGVRSISTQVVRNRGGMSDNDTLFWRMLALFQSLPDLQPAQIVDWLAQESGETLTPERLATLTQPQLAASFPSATAVMSPARWSRVMASLQGALPAHLRIVRPAQRTPQLLAAFCSQDGLVINGHFGQGRLFFIYAFDEQGGWLYDLRRYPSAPHQQEANEVRARLIEDCQLLFCQEIGGPAAARLIRHRIHPMKAQPGTTIQAQCEAINTLLAGRLPPWLAKRLNRDNPLEERVFGGYPYDVPDYA

SEQ ID NO: 145 - protein, 213 aa, Artificial Sequence
  Amino acid sequence of the MTP-FA 51::NifZ::HA fusion polypeptide encoded by SN146. Amino acids 1-54 correspond to the MTP-FA 51 with GG, amino acids 55-202 correspond to K. oxytoca NifZ (SEQ ID NO16), and amino acids 203-213 include the HA epitope.
MMAMAVFRREGRRLLPSIAARPIAAIRSPLSSDQEEGLLGVRSISTQVVRNRGGMRPKFTFSEEVRVVRAIRNDGTVAGFAPGALLVRRGSTGFVRDWGVFLQDQIIYQIHFPETDRIIGCREQELIPITQPWLAGNLQYRDSVTCQMALAVNGDVVVSAGQRGRVEATDRGELGDSYTVDFSGRWFRVPVQAIALIEEREEGGYPYDVPDYA

SEQ ID NO: 146 - protein, 1085 aa, Artificial Sequence
  Amino acid sequence of MTP-FA 51::NifD(Y100Q)::linker(HA)::NifK fusion polypeptide encoded by SN159. Amino acids 1-54 correspond to the MTP-FA 51 with GG at its C-terminus, amino acids 55-536 correspond to K. oxytoca NifD with the Y100Q substitution, amino
MMAMAVFRREGRRLLPSIAARPIAAIRSPLSSDQEEGLLGVRSISTQVVRNRGGMTNATGERNLALIQEVLEVFPETARKERRKHMMVSDPEMESVGKCIISNRKSQPGVMTVRGCAYAGSKGVVFGPIKDMAHISHGPVGCGQYSRAGRRNQYTGVSGVDSFGTLNFTSDFQERDIVFGGDKKLSKLIEEMELLFPLTKGITIQSECPVGLIGDDISAVANASSKALDKPVIPVRCEGFRGVSQSLGHHIANDVVRDWILNNREGQPFETTPYDVAIIGDYNIGGDAWASRILLEEMGLRVVAQWSGDGTLVEMENTPFVKLNLVHCYRSMNYIARHMEEKHQIPWMEYNFFGPTKIAESLRKIADQFDDTIRANAEAVIARYEGQMAAIIAKYRPRLEGRKVLLYMGGLRPRHVIGAYEDLGMEIIAAGYEFAHNDDYDRTLPDLKEGTLLFDDASSYELEAFVKALKPDLIGSGIKEKYIFQKMGVPFRQMHSWDYSGPYHGYDGFAIFARDMDMTLNNPAWNELTAPWLKSAATPPPGSTTTAYPYDVPDYATPPPGSTTTASQTIDKINSCYPLFEQDEYQELFRNKRQLEEAHDAQRVQEVFAWTTTAEYEALNFQREALTVDPAKACQPLGAVLCSLGFANTLPYVHGSQGCVAYFRTYFNRHFKEPIACVSDSMTEDAAVFGGNNNMNLGLQNASALYKPEIIAVSTTCMAEVIGDDLQAFIANAKKDGFVDSSIAVPHAHTPSFIGSHVTGWDNMFEGFAKTFTADYQGQPGKLPKLNLVTGFETYLGNFRVLKRMMEQMAVPCSLLSDPSEVLDTPADGHYRMYSGGTTQQEMKEAPDAIDTLLLQPWQLLKSKKVVQEMWNQPATEVAIPLGLAATDELLMTVSQLSGKPIADALTLERGRLVDMMLDSHTWLHGKKFGLYGDPDFVMGLTRFLLELGCEPTVILSHNANKRWQKAMNKMLDASPYGRDSEVFINCDLWHFRSLMFTRQPDFMIGNSYGKFIQRDTLAKGKAFEVPLIRLGFPLFDRHHLHRQTTWGYEGAMNIVTTLVNAVLEKLDSDTSQLGKTDYSFDLVR

SEQ ID NO: 147 - protein, 533 aa, Artificial Sequence
  Amino acid sequence of the MTP-FA 51::NifB::HA fusion polypeptide encoded by SN192. Amino acids 1-54 correspond to the MTP-FA 51 with GG, amino acids 55-522 correspond to K. oxytoca NifB according to Temme et al., (2012), and amino acids 523-533
MMAMAVFRREGRRLLPSIAARPIAAIRSPLSSDQEEGLLGVRSISTQVVRNRGGMTSCSSFSGGKACRPADDSALTPLVADKAAAHPCYSRHGHHRFARMHLPVAPACNLQCNYCNRKFDCSNESRPGVSSTLLTPEQAVVKVRQVAQAIPQLSVVGIAGPGDPLANIARTFRTLELIREQLPDLKLCLSTNGLMLPDAVDRLLDVGVDHVTVTINTLDAEIAAQIYAWLWLDGERYSGREAGEILIARQLEGVRRLTAKGVLVKINSVLIPGINDSGMADVSRALRASGAFIHNIMPLIARPEHGTVFGLNGQPEPDAETLAATRSRCGEVMPQMTHCHQCRADAIGMLGEDRSQQFTQLPAPESLPAWLPILHQRAQLHASIATRGESEADDACLVAVASSRGDVIDCHFGHADRFYIYSLSAAGMVLVNERFTPKYCQGRDDCEPQDNAARFAAILELLADVKAVFCVRIGHTPWQQLEQEGIEPCVDGAWRPVSEVLPAWWQQRRGSWPAALPHKGVAGGYPYDVPDYA

SEQ ID NO: 148 - protein, 479 aa, Azospirillum brasilense
MSLSVNEGVDVKGLVDKVLEAYPEKSRKRRAKHLNVLEAEAKDCGVKSNIKSIPGVMTIRGCAYAGSKGVVWGPIKDMIHISHGPVGCGYYSWSGRRNYYVGDTGVDSWGTMHFTSDFQEKDIVFGGDKKLHKVIEEINELFPLVNGISIQSECPIGLIGDDIEAVARAKSEELGKPVVPVRCEGFRGVSQSLGHHIANDVIRDWIFEKTEPKEGFVSTPYDVTIIGDYNIGGDAWASRILLEEIGLRVIAQWSGDGTLAELENTPKAKVNLIHCYRSMNYIARHMEEKFGIPWMEYNFFGPSQIAESLRKIAALFDDTIKENAEKVIAKYQPMVDAVIAKFKPRLEGKKVMIYVGGLRPRHVVDAYHDLGMEIVGTGYEFAHNDDYQRTQHYVKEGTLIYDDVTAFELEKFVEVMRPDLVASGIKEKYVFQKMGLPFRQMHSWDYSGPYHGYDGFAIFARDMDLAINNPVWGIMKAPF

SEQ ID NO: 149 - protein, 492 aa, Azotobacter vinelandii
MTGMSREEVESLIQEVLEVYPEKARKDRNKHLAVNDPAVTQSKKCIISNKKSQPGLMTIRGCAYAGSKGVVWGPIKDMIHISHGPVGCGQYSRAGRRNYYIGTTGVNAFVTMNFTSDFQEKDIVFGGDKKLAKLIDEVETLFPLNKGISVQSECPIGLIGDDIESVSKVKGAELSKTIVPVRCEGFRGVSQSLGHHIANDAVRDWVLGKRDEDTTFASTPYDVAIIGDYNIGGDAWSSRILLEEMGLRCVAQWSGDGSISEIELTPKVKLNLVHCYRSMNYISRHMEEKYGIPWMEYNFFGPTKTIESLRAIAAKFDESIQKKCEEVIAKYKPEWEAVVAKYRPRLEGKRVMLYIGGLRPRHVIGAYEDLGMEVVGTGYEFAHNDDYDRTMKEMGDSTLLYDDVTGYEFEEFVKRIKPDLIGSGIKEKFIFQKMGIPFRQMHSWDYSGPYHGFDGFAIFARDMDMTLNNPCWKKLQAPWEASEGAEKVAASA

SEQ ID NO: 150 - protein, 504 aa, Sinorhizobium fredii
MSLDYENDSALHQELITQVLSQYPHKAAKRRQKHLSVASDREAVGEEGETLSECDVKSNIKSIPGVMTIRGCAYAGSKGVVWGPVKDMVHISHGPVGCGQYSWSQRRNYYVGTTGVDTFVTMQFTSDFQEKDIVFGGDKKLEQVIDEIEELFPLNNGITIQSECPIGLIGDDIEAVSRKKAAEHETTIVPVRCEGFRGVSQSLGHHIANDAIRDWVFDKADGKTDVEFETGPYDVNVIGDYNIGGDAWASRILLEEIGLRVVGNWSGDATLAEVERAPRAKLNLIHCYRSMNYICRHMEERYAIPWMEYNFFGPSQIEASLRKIARHFGPTIEERAERVIAKYRPLVDAVIDKYWPRLQGKRVMLYVGGLRPRHVITAYEDLGMQIVGTGYEFAHNDDYQRTGHYVKTGTLIYDDATSYELDTFIERIRPDLVGSGIKEKYPVQKMGIPFRQMHSWDYSGPYHGYDGFAIFARDMDLAINNPVWDLYDAPWKKMTVPTAAVAAE

SEQ ID NO: 151 - protein, 543 aa, Chlorobium tepidum
MEAKVLIPDPSKIKEELINKYPAKVAKKRSKSIVVNDPEIVPEVQANVRTVPGIITQRGCAYAGCKGVVLGPTRDIVNIVHGPIGCSFYAWLTRRNQTRPETPEHENYITYCFSTDMQEEHVVFGGEKKLKVAIQEAYDLFHPKAIAIFSTCPVGLIGDDVHAVAREMKEKLGDCNVFGFSCEGYRGVSQSAGHHIANNGVFKHMVGNNNEVKPGKFKLNLLGEYNIGGDAFEIERLLEKCGITLVASFSGNSTVGAIENAHTADLNVIMCHRSINYMGDMMETKYGIPWMKVNFVGAESTAKSLRKIAEYFGDEELKAKVEEVIAEEVPAVKAIIDEIRPRTEGKTAMLFVGGSRAHHYQDLFSELGMTTIAAGYEFAHRDDYEGREVLPKIKIDADSKNIEELKVTADPELYNPRKSKAELEELKAKGLEINGYEGMMKQMMKKTLVVDDISHYESEKLIEMYKPDIFCAGIKEKYVVQKMGVPLKQLHSYDYGGPYTGFKGAVNFYKDIDRMVNNPVWKMIKAPWEKSEPESLEASYVAS

SEQ ID NO: 152 - protein, 544 aa, Desulfovibrio vulgaris
MALKHKSIPDVATVKEELLKKYPTKVARKRAKQIVINDVKDGDVVPEVQANVRTTPGIITMRGCTYAGCKGVILGPTRDIVNITHGPIGCGFYSWLTRRNQTKAPLESSENFMPYAFSTDMQDEDIIFGGEKKLIAAIQEAYDTFHPKAIAIFATCPVGLIGDDIHAVARKMKEKLGINIFAFSCEGYKGVSQSAGHHIANNQIFTHVVGEDDTPKLGEYKINMLGEYNIGGDAFELERVLEKCGITLVSTFSGNSTYEHFATAHQADLNAVMCHRSINYVAEMMETKYGIPWIKVNFIGAESSAKSLRKIAQYFGDKKLIDRVEEVIAEEMPAVHAALEDVKPFTEGKTAMLFVGGSRAHHYQDLFTEMGMKTIAAGYEFAHRDDYEGRKVMPTIKVDADSRNIEEIEVTPDSTRFVPRKSDEDLKRLAEAGFTFKDYEGMMPQMESDTLVIDDLNQYEADKLIELLKPDVFCAGIKEKFSVQKMGVPMKQLHSYDYGGPYAGFKGAVNFYTEIKRLVTSKVWSDLKAPWEENPELSATYVWE

SEQ ID NO: 153 - protein, 538 aa, Artificial Sequence
  Amino acid sequence of wild-type Desulfotomaculum ferrireducens NifD polypeptide, 539aa
MAINEKVLDEILSQYPTKVKKNRKKHIIIKDPNQARQEIEANTRTIPGIITNRGCAYAGCKGVVLGPLKDVVHITHGPIGCGYYSWLTRRNKAASSDPTKNFISYCFSTDMQESDIVFGGEKKLARMIDEVMEIFKPNAITISATCPVGLIGDDLGAVAKAAEQKHGITVMHFNCEGYKGVSQSAGHHIANNTLMERVIGAGELEAAPGRYPINILGEYNIGGDSWEIERILREIGYTVLSVMTGDGSYEELKNAHVAELNLVQCHRSINYIAEMLETKYGTPWLKVNFIGIQSTIDSLRNMAIYFGDPELTRRTEEVIAKELAEVEPVMEQYKKICQGKTAFCFVGGSRGHHYQGLFAELGMETVLAGYEFAHRDDYEGRDVLPQIKLDADNKNIPELHVEPDQRRFKLKVPRERMEELKKKIPLSYYAGMMVDMKGGHVVVDDLNHYETEQFIKLLKPDIFASGIKDKYVVQKMGIPAKQLHSYDYSGPYAGFKGAVKFAEDITMSFISPTWNFITPPWKNQPILEGEIVEGGCST

SEQ ID NO: 154 - protein, 4 aa, Artificial Sequence
  peptide sequence
  X (4)..(4): any amino acid other than Y
RRNX

SEQ ID NO: 155 - protein, 9 aa, Artificial Sequence
  Tryptic peptide sequence from NifM
DAFAPLAQR

SEQ ID NO: 156 - protein, 10 aa, Artificial Sequence
  Tryptic peptide sequence from NifM
DYLWQQSQQR

SEQ ID NO: 157 - protein, 14 aa, Artificial Sequence
  Tryptic peptide sequence from CAT polypeptide
ITGYTTVDISQWHR

SEQ ID NO: 158 - protein, 9 aa, Artificial Sequence
  Tryptic peptide sequence from CAT polypeptide
LMNAHPEFR

SEQ ID NO: 159 - protein, 7 aa, Artificial Sequence
  Tryptic peptide sequence from CAT polypeptide
YYTQGDK

SEQ ID NO: 160 - protein, 358 aa, Artificial Sequence
  Amino acid sequence of the MTP-FA?51::NifU::TwinStrep fusion polypeptide encoded by SN166. Amino acids 1-54 are the MTP-FA?51 sequence with an additional methionine translational start and C-terminal GG, amino acids 55-328 are the NifU sequence, and
MMAMAVFRREGRRLLPSIAARPIAAIRSPLSSDQEEGLLGVRSISTQVVRNRGGMWNYSEKVKDHFFNPRNARVVDNANAVGDVGSLSCGDALRLMLRVDPQSEIIEEAGFQTFGCGSAIASSSALTELIIGHTLAEAGQITNQQIADYLDGLPPEKMHCSVMGQEALRAAIANFRGESLEEEHDEGKLICKCFGVDEGHIRRAVQNNGLTTLAEVINYTKAGGGCTSCHEKIELALAEILAQQPQTTPAVASGKDPHWQSVVDTIAELRPHIQADGGDMALLSVTNHQVTVSLSGSCSGCMMTDMTLAWLQQKLMERTGCYMEVVAAGGWSHPQFEKGGGSGGGSGGSAWSHPQFEK

SEQ ID NO: 161 - protein, 484 aa, Artificial Sequence
  Amino acid sequence of the MTP-FA?51::NifS::TwinStrep fusion polypeptide encoded by SN231. Amino acids 1-54 are the MTP-FA?51 sequence with an additional methionine translational start and C-terminal GG, amino acids 55-454 are the NifS sequence, and
MMAMAVFRREGRRLLPSIAARPIAAIRSPLSSDQEEGLLGVRSISTQVVRNRGGMKQVYLDNNATTRLDPMVLEAMMPFLTDFYGNPSSIHDFGIPAQAALERAHQQAAALLGAEYPSEIIFTSCATEATATAIASAIALLPERREIITSVVEHPATLAACEHLERQGYRIHRIAVDSEGALDMAQFRAALSPRVALVSVMWANNETGVLFPIGEMAELAHEQGALFHCDAVQVVGKIPIAVGQTRIDMLSCSAHKFHGPKGVGCLYLRRGTRFRPLLRGGHQEYGRRAGTENICGIVGMGAACELANIHLPGMTHIGQLRNRLEHRLLASVPSVMVMGGGQPRVPGTVNLAFEFIEGEAILLLLNQAGIAASSGSACTSGSLEPSHVMRAMNIPYTAAHGTIRFSLSRYTREKEIDYVVATLPPIIDRLRALSPYWQNGKPRPADAVFTPVYGGGWSHPQFEKGGGSGGGSGGSAWSHPQFEK

SEQ ID NO: 162 - protein, 9 aa, Artificial Sequence
  Peptide sequence
ISTQVVRNM

SEQ ID NO: 163 - protein, 384 aa, Artificial Sequence
  Amino acid sequence of the NifV polypeptide from A. vinelandii (AvNifV; Accession No. CP001157); 384aa.
MASVIIDDTTLRDGEQSAGVAFNADEKIAIARALAELGVPELEIGIPSMGEEEREVMHAIAGLGLSSRLLAWCRLCDVDLAAARSTGVTMVDLSLPVSDLMLHHKLNRDRDWALREVARLVGEARMAGLEVCLGCEDASRADLEFVVQVGEVAQAAGARRLRFADTVGVMEPFGMLDRFRFLSRRLDMELEVHAHDDFGLATANTLAAVMGGATHINTTVNGLGERAGNAALEECVLALKNLHGIDTGIDTRGIPAISALVERASGRQVAWQKSVVGAGVFTHEAGIHVDGLLKHRRNYEGLNPDELGRSHSLVLGKHSGAHMVRNTYRDLGIELADWQSQALLGRIRAFSTRTKRSPQPAELQDFYRQLCEQGNPELAAGGMA

SEQ ID NO: 164 - protein, 380 aa, Artificial Sequence
  Amino acid sequence of the KoNifV variant sequence
MERVLINDTTLRDGEQSPGVAFRTSEKVAIAEALYAAGITAMEVGTPAMGDEEIARIQLVRRQLPDATLMTWCRMNALEIRQSADLGIDWVDISIPASDKLRQYKLREPLAVLLERLAMFIHLAHTLGLKVCIGCEDASRASGQTLRAIAEVAQQCAAARLRYADTVGLLDPFTTAAQISALRDVWSGEIEMHAHNDLGMATANTLAAVSAGATSVNTTVLGLGERAGNAALETVALGLERCLGVETGVHFSALPALCQRVAEAAQRAIDPQQPLVGELVFTHESGVHVAALLRDSESYQSIAPSLMGRSYRLVLGKHSGRQAVNGVFDQMGYHLNAAQINQLLPAIRRFAENWKRSPKDYELVAIYDELCGESALRARG

SEQ ID NO: 165 - protein, 12 aa, Artificial Sequence
  N-terminal extension (scar sequence).
MYPYDVPDYAGG

SEQ ID NO: 166 - protein, 22 aa, Artificial Sequence
  N-terminal AvNifV extension (scar sequence)
ISTQVVRNRGGYPYDVPDYAGG

SEQ ID NO: 167 - protein, 330 aa, Artificial Sequence
  Amino acid sequence of the MTP-FA?51::HA::KoNifM polypeptide encoded by SN43. Amino acids 1-53 correspond to the MTP-FA?51 sequence including a GG at its C-terminus, amino acids 54-64 correspond to the HA epitope including a GG at its C-terminus,
MAMAVFRREGRRLLPSIAARPIAAIRSPLSSDQEEGLLGVRSISTQVVRNRGGYPYDVPDYAGGMNPWQRFARQRLARSRWNRDPAALDPADTPAFEQAWQRQCHMEQTIVARVPEGDIPAALLENIAASLAIWLDEGDFAPPERAAIVRHHARLELAFADIARQAPQPDLSTVQAWYLRHQTQFMRPEQRLTRHLLLTVDNDREAVHQRILGLYRQINASRDAFAPLAQRHSHCPSALEEGRLGWISRGLLYPQLETALFSLAENALSLPIASELGWHLLWCEAIRPAAPMEPQQALESARDYLWQQSQQRHQRQWLEQMISRQPGLCG

SEQ ID NO: 168 - protein, 354 aa, Artificial Sequence
  Amino acid sequence of the MTP-CoxIV::TwinStrep::NifH polypeptide encoded by SN178. Amino acids 1-31 correspond to the MTP-CoxIV sequence, amino acids 32-61 correspond to the TwinStrep sequence including a GG at its C-terminus, and amino acids 62-354
MLSLRQSIRFFKPATRTLCSSRYLLQQKPSAWSHPQFEKGGGSGGGSGGSAWSHPQFEKGGMSLRQIAFYGKGGIGKSTTSQNTLAALVELDQKILIVGCDPKADSTRLILHAKAQDTVLHLAAEAGSVEDLELEDVLKIGYKGIKCVESGGPEPGVGCAGRGVITSINFLEENGAYDDVDYVSYDVLGDVVCGGFAMPIRENKAQEIYIVMSGEMMALYAANNIAKGILKYAHSGGVRLGGLICNERQTDKEIDLASALAARLGTQLIHFVPRDNIVQHAELRRMTVIEYAPDSQQAQEYRQLANKVHANKGKGTIPTPITMEELEEMLMDFGIMKSEEQQLAELQAKEAAKA

SEQ ID NO: 169 - protein, 356 aa, Artificial Sequence
  Amino acid sequence of the MTP-CoxIV::TwinStrep::NifH polypeptide encoded by SN179. Amino acids 1-31 correspond to the MTP-CoxIV sequence, amino acids 32-61 correspond to the TwinStrep sequence including a GG at its C-terminus, and amino acids 62-356
MLSLRQSIRFFKPATRTLCSSRYLLQQKPSAWSHPQFEKGGGSGGGSGGSAWSHPQFEKGGMTENIRQIAFYGKGGIGKSTTSQNTLAAMAEMGQRIMIVGCDPKADSTRLMLHSKAQTTVLHLAAERGAVEDLELEEVMLTGFRGVKCVESGGPEPGVGCAGRGIITAINFLEENGAYQDLDFVSYDVLGDVVCGGFAMPIREGKAQEIYIVTSGEMMAMYAANNIARGILKYAHSGGVRLGGLICNSRKVDREAELIENLAERLNTQMIHFVPRDNIVQHAELRRMTVNEYAPDSNQSQEYRALAKKIINNTKLTIPTPMEMDELEALLIEYGILDDDTKHADIIGKPAEASAK

SEQ ID NO: 170 - protein, 348 aa, Artificial Sequence
  Amino acid sequence of the MTP-CoxIV::TwinStrep::NifH polypeptide encoded by SN180. Amino acids 1-31 correspond to the MTP-CoxIV sequence, amino acids 32-61 correspond to the TwinStrep sequence including a GG at its C-terminus, and amino acids 62-338
MLSLRQSIRFFKPATRTLCSSRYLLQQKPSAWSHPQFEKGGGSGGGSGGSAWSHPQFEKGGMRQIAFYGKGGIGKSTTQQNTMAAMAEMGKKVMIVGCDPKADSTRLILHSKAQTSVIQLAAEKGSVEDLELDEVLVEGQWGIKCVESGGPEPGVGCAGRGVITSISYLEEAGAYEDLDFVTYDVLGDVVCGGFAMPIRQGKAQEIYIVTSGEMMAMYAANNIARGILKYAHSGGVRLGGLICNSRNTDREDELIIELARRLNTQMIHFIPRNNVVQHAELRRMTVIEYDPKNEQADQYRQLAKKIVDNDMKTIPTPITMDELEELLIEFGIMEQEDESIIGKAAAVA

SEQ ID NO: 171 - protein, 354 aa, Artificial Sequence
  Amino acid sequence of the MTP-CoxIV::TwinStrep::NifH polypeptide encoded by SN181. Amino acids 1-31 correspond to the MTP-CoxIV sequence, amino acids 32-61 correspond to the TwinStrep sequence including a GG at its C-terminus, and amino acids 62-338
MLSLRQSIRFFKPATRTLCSSRYLLQQKPSAWSHPQFEKGGGSGGGSGGSAWSHPQFEKGGMATRQCAIYGKGGIGKSTTTQNLVAGLAELGKRVMIVGCDPKADSTRLILHSKAQETIMQMAADAGSVEDLELEDVLKVGFGDIKCVESGGPEPGVGCAGRGVITAINFLEEEGAYEEDLDFVFYDVLGDVVCGGFAMPIRENKAQEIYIVCSGEMMAMYAANNIAKGIVKYASSGGVRLAGLICNSRNTAREDELIMELARQLGTQMIHFVPRDNIVQRAEIRRMTVIEYDPKSGQADEYRTLAQKIIDNKMFVVPTPISMDALEDLLMEFGVLEEEDESIVGKTAAEEAVA

SEQ ID NO: 172 - protein, 345 aa, Artificial Sequence
  Amino acid sequence of the MTP-CoxIV::TwinStrep::NifH polypeptide encoded by SN182. Amino acids 1-31 correspond to the MTP-CoxIV sequence, amino acids 32-61 correspond to the TwinStrep sequence including a GG at its C-terminus, and amino acids 62-345
MLSLRQSIRFFKPATRTLCSSRYLLQQKPSAWSHPQFEKGGGSGGGSGGSAWSHPQFEKGGMSFDEIAPNAKKVAIYGKGGIGKSTTTQNTAAALAYYYKLKGMIHGCDPKADSTRMILHGKPQETVMDVLREEGEEGVTLEKVRKVGFCGIYCVESGGPEPGVGCAGRGVITAVNLMKELGGYPDDLDFLFFDVLGDVVCGGFAMPLRDGLAKEIYIVSSGEMMALYAANNIAKGILKYAEQSGVRLGGIICNSRNVDGERELMEEFCDKLGTKLIHFIPRDNIVQKAEFNKMTVVEFAPDHPQALEYKKLGKKIMDNDELVIPTPLSMDELEKLVEKYGLYDK

SEQ ID NO: 173 - protein, 345 aa, Artificial Sequence
  Amino acid sequence of the MTP-CoxIV::TwinStrep::NifH polypeptide encoded by SN183. Amino acids 1-31 correspond to the MTP-CoxIV sequence, amino acids 32-61 correspond to the TwinStrep sequence including a GG at its C-terminus, and amino acids 62-345
MLSLRQSIRFFKPATRTLCSSRYLLQQKPSAWSHPQFEKGGGSGGGSGGSAWSHPQFEKGGMRQIAIYGKGGIGKSTTTQNTVSALAEMGKKVMIVGCDPKADSTRLILHSKAQATVMDLAREKGTVEDLELSDVLLTGFADIRCAESGGPEPGVGCAGRGVITAINFLEENGAYTPDLDYVFYDVLGDVVCGGFAMPIRENKAQEIYIVTSGEMMAMYAANNIARGILKYASSGKVRLGGLICNSRKVDKEYELIDELATRLGTQMIHFLPRDNQVQRAELRRMTVIEYSPDHPQADEYRALAKKIDENKKLVIPTPLTMDELEDLLIQYGILEDEETAAAKLG

SEQ ID NO: 174 - protein, 335 aa, Artificial Sequence
  Amino acid sequence of the MTP-CoxIV::TwinStrep::NifH polypeptide encoded by SN184. Amino acids 1-31 correspond to the MTP-CoxIV sequence, amino acids 32-61 correspond to the TwinStrep sequence including a GG at its C-terminus, and amino acids 62-335
MLSLRQSIRFFKPATRTLCSSRYLLQQKPSAWSHPQFEKGGGSGGGSGGSAWSHPQFEKGGMRKVAIYGKGGIGKSTTTQNTVAGLAEAGKKVMVVGCDPKADSTRLLLGGLQQKTVLDTLREEGEEVELEDIIKEGYRNTRCTESGGPEPGVGCAGRGIITSVNLLEQLGAYDDEWELDYVFYDVLGDVVCGGFAMPIRDGKAEEIYIVCSGEMMAMYAANNICKGILKYADAGGVRLGGLICNSRKVDNEREMIEELARRLGTQMIHFVPRDNFVQRAEINRKTVIDFDPTHPQADEYRALAKKIDENKMFVIPKPLEIDELESLLIEFGIAN

SEQ ID NO: 175 - protein, 350 aa, Artificial Sequence
  Amino acid sequence of the MTP-CoxIV::TwinStrep::NifH polypeptide encoded by SN185. Amino acids 1-31 correspond to the MTP-CoxIV sequence, amino acids 32-61 correspond to the TwinStrep sequence including a GG at its C-terminus, and amino acids 62-350
MLSLRQSIRFFKPATRTLCSSRYLLQQKPSAWSHPQFEKGGGSGGGSGGSAWSHPQFEKGGMRQIAIYGKGGIGKSTTTQNTVAGLASIGKKVMIVGCDPKADSTRLILHAKAQSTVMDLVRELGTVEDLELEDVMKVGYGDVKCVESGGPEPGVGCAGRGVITAINFLEENGAYTPDLDFVFYDVLGDVVCGGFAMPIREGKAEEIYIVCSGEMMAMYAANNIAKGILKYATSGKVRLAGLICNARKTDKEYELIDALAKKLGTQMIHFVPRDNQVQRAELRRMTVIEYSPEHPQAQEYRTLAQKIADNKMLVVPTPLEMEELEDLLMEFGIMEAEDESIVGVAEAAAV

SEQ ID NO: 176 - protein, 355 aa, Artificial Sequence
  Amino acid sequence of the MTP-CoxIV::TwinStrep::NifH polypeptide encoded by SN186. Amino acids 1-31 correspond to the MTP-CoxIV sequence, amino acids 32-61 correspond to the TwinStrep sequence including a GG at its C-terminus, and amino acids 62-355
MLSLRQSIRFFKPATRTLCSSRYLLQQKPSAWSHPQFEKGGGSGGGSGGSAWSHPQFEKGGMASLRQIAFYGKGGIGKSTTSQNTLAALAEMGQKILIVGCDPKADSTRLILHAKAQDTILSLAASAGSVEDLELEDVMKVGYQDIRCVESGGPEPGVGCAGRGVITSINFLEENGAYENIDYVSYDVLGDVVCGGFAMPIRENKAQEIYIVMSGEMMAMYAANNISKGILKYANSGGVRLGGLICNERQTDKELELAEALAKKLGTQLIYFVPRDNVVQHAELRRMTVLEYAPDSKQADHYRKLAAKVHNNGGKGIIPTPISMDELEDMLMEHGIIKAVDESIIGKTAAELAAS

SEQ ID NO: 177 - protein, 336 aa, Artificial Sequence
  Amino acid sequence of the MTP-CoxIV::TwinStrep::NifH polypeptide encoded by SN187. Amino acids 1-31 correspond to the MTP-CoxIV sequence, amino acids 32-61 correspond to the TwinStrep sequence including a GG at its C-terminus, and amino acids 62-336
MLSLRQSIRFFKPATRTLCSSRYLLQQKPSAWSHPQFEKGGGSGGGSGGSAWSHPQFEKGGMVRKIAIYGKGGIGKSTTQQNTAAAMSYFHGKNVMIHGCDPKADSTRLILGGKMQTTMMDTLRELGEVACTPDKVIETGFGGIKCVESGGPEPGVGCAGRGVITAITLMERHGVYEKDLDFVFFDVLGDVVCGGFAMPVRDGKAEEIYIVASGEMMALYAANNICKGMVKYARQSGVRLGGIICNSRNVDGEKELLEEFCERIGTQMIHFVPRDNIVQKAEFNKKSVIEFDPECNQSQEYRELARKIIENKDFVIPEPMTMDEMEELVVKYGVMD

SEQ ID NO: 178 - protein, 334 aa, Artificial Sequence
  Amino acid sequence of the MTP-CoxIV::TwinStrep::NifH polypeptide encoded by SN188. Amino acids 1-31 correspond to the MTP-CoxIV sequence, amino acids 32-61 correspond to the TwinStrep sequence including a GG at its C-terminus, and amino acids 62-334
MLSLRQSIRFFKPATRTLCSSRYLLQQKPSAWSHPQFEKGGGSGGGSGGSAWSHPQFEKGGMRQIAIYGKGGIGKSTTTQNLTASLSTMGNKIMLVGCDPKADSTRMLLGGLNQKTVLDTLRSEGDEGVDLDVVMQRGFGDIKCVESGGPEPGVGCAGRGIITSIGLLENLGAYTDDLDYVFYDVLGDVVCGGFAMPIREGKAKEIYIVASGELMAIYAANNICKGLAKFAKGGARLGGIICNSRNVDGERELLDAFAKKLGSHLIHFIPRDNIVQRAEINRKTVIDFDPESNQAKEYLTLAHNVQNNDKLVVPTPLPMEELEAMMVEFGIVDL

SEQ ID NO: 179 - protein, 336 aa, Artificial Sequence
  Amino acid sequence of the MTP-CoxIV::TwinStrep::NifH polypeptide encoded by SN189. Amino acids 1-31 correspond to the MTP-CoxIV sequence, amino acids 32-61 correspond to the TwinStrep sequence including a GG at its C-terminus, and amino acids 62-336
MLSLRQSIRFFKPATRTLCSSRYLLQQKPSAWSHPQFEKGGGSGGGSGGSAWSHPQFEKGGMRQIAIYGKGGIGKSTTTQNTVAALADAGKKIMVVGCDPKADSTRLLLNGLNQKTVLDTLRDEGEDVILEDVLRTGFKDVKCVESGGPEPGVGCAGRGIITSINLLESLGAYTDDLDYVFYDVLGDVVCGGFAMPIREGKAREIYIVASGELMALYAANNICKGVQKYAKTGGVRMGGIICNSRKVDKEYDLLKAFAEEIGTQLIHFLPRDNVVQRAEIKKKTVIDYDPTVAQADEYRKLAKNIDENTMFVIPNPMTQDRLEELMMEHGFMEGLD

SEQ ID NO: 180 - protein, 336 aa, Artificial Sequence
  Amino acid sequence of the MTP-CoxIV::TwinStrep::NifH polypeptide encoded by SN190. Amino acids 1-31 correspond to the MTP-CoxIV sequence, amino acids 32-61 correspond to the TwinStrep sequence including a GG at its C-terminus, and amino acids 62-336
MLSLRQSIRFFKPATRTLCSSRYLLQQKPSAWSHPQFEKGGGSGGGSGGSAWSHPQFEKGGMTRKIAIYGKGGIGKSTTQQNTAAALAYFYGKKVLIHGCDPKADCTRLILGGKPQETVMDTMRELGEDAVTIDRVVKTGFCGIKCVESGGPEPGVGCAGRGVITAISLMEELGAYTPDLDFIFFDVLGDVVCGGFAMPVREGKAQEIYIVASGEMMALYAANNICRGMVKYAEQSGVRLGGIICNSRNVDGERELMEEFCSKIGTQMIHFIPRDNIVQKAEFNRQTVTQFDPNCNQAQEYRELARKIIENDMFVIPKPMTMDEMENLVIKYGLLE

SEQ ID NO: 181 - protein, 335 aa, Artificial Sequence
  Amino acid sequence of the MTP-CoxIV::TwinStrep::NifH polypeptide encoded by SN191. Amino acids 1-31 correspond to the MTP-CoxIV sequence, amino acids 32-61 correspond to the TwinStrep sequence including a GG at its C-terminus, and amino acids 62-335
MLSLRQSIRFFKPATRTLCSSRYLLQQKPSAWSHPQFEKGGGSGGGSGGSAWSHPQFEKGGMRKIAIYGRGGVGKSTTTQNVVAGLSEMSRKVMVVGCDSKADSTRLLLGGLHQKIVLDTLRKEEDDVNLEDFRLEGWGKTLCVESGGPEPGVGCTGRGILTSIGLLEQLGAYDDAVRLDYTFYDGLGDVVCSGFVMPIRERKAQEIYIVTSGEIMAMYTANNICRSLQKYAPVGGIRLGGLICNSRKVDRENDLVEALAEKLGTQTIYSIPRDNMVQRAEFYRKTVIEYAPECEQAQHYRNLAAAIDQNTDFVIPKSMSSDRLEELLVKFGLFD

SEQ ID NO: 182 - protein, 583 aa, Artificial Sequence
  MTP-FA?51::AnfD::HA polypeptide
MMAMAVFRREGRRLLPSIAARPIAAIRSPLSSDQEEGLLGVRSISTQVVRNRGGMPHHEFECSKVIPERKKHAVIKGKGETLADALPQGYLNTIPGSISERGCAYCGAKHVIGTPMKDVIHISHGPVGCTYDTWQTKRYISDNDNFQLKYTYATDVKEKHIVFGAEKLLKQNIIEAFKAFPQIKRMTIYQTCATALIGDDINAIAEEVMEEMPEVDIFVCNSPGFAGPSQSGGHHKINIAWINQKVGTVEPEITGDHVINYVGEYNIQGDQEVMVDYFKRMGIQVLSTFTGNGSYDGLRAMHRAHLNVLECARSAEYICNELRVRYGIPRLDIDGFGFKPLADSLRKIGMFFGIEDRAKAIIDEEVARWKPELDWYKERLMGKKVCLWPGGSKLWHWAHVIEEEMGLKVVSVYTKFGHQGDMEKGIARCGEGTLAIDDPNELEGLEALEMLKPDIILTGKRPGEVAKKVRVPYLNAHAYHNGPYKGFEGWVRFARDIYNAIYSPIHQLSGIDITKDNAPEWGNGFRTRQMLSDGNLSDAVRNSETLRQYTGGYDSVSKLREREYPAFERKVGGGYPYDVPDYA

SEQ ID NO: 183 - protein, 530 aa, Artificial Sequence
  HA::AnfD polypeptide
MYPYDVPDYAGGMPHHEFECSKVIPERKKHAVIKGKGETLADALPQGYLNTIPGSISERGCAYCGAKHVIGTPMKDVIHISHGPVGCTYDTWQTKRYISDNDNFQLKYTYATDVKEKHIVFGAEKLLKQNIIEAFKAFPQIKRMTIYQTCATALIGDDINAIAEEVMEEMPEVDIFVCNSPGFAGPSQSGGHHKINIAWINQKVGTVEPEITGDHVINYVGEYNIQGDQEVMVDYFKRMGIQVLSTFTGNGSYDGLRAMHRAHLNVLECARSAEYICNELRVRYGIPRLDIDGFGFKPLADSLRKIGMFFGIEDRAKAIIDEEVARWKPELDWYKERLMGKKVCLWPGGSKLWHWAHVIEEEMGLKVVSVYTKFGHQGDMEKGIARCGEGTLAIDDPNELEGLEALEMLKPDIILTGKRPGEVAKKVRVPYLNAHAYHNGPYKGFEGWVRFARDIYNAIYSPIHQLSGIDITKDNAPEWGNGFRTRQMLSDGNLSDAVRNSETLRQYTGGYDSVSKLREREYPAFERKVG

SEQ ID NO: 184 - protein, 526 aa, Artificial Sequence
  MTP-FA?51::HA::AnfK polypeptide
MAMAVFRREGRRLLPSIAARPIAAIRSPLSSDQEEGLLGVRSISTQVVRNRGGYPYDVPDYAGGMTCEVKEKGRVGTINPIFTCQPAGAQFVSIGIKDCIGIVHGGQGCVMFVRLIFSQHYKESFELASSSLHEDGAVFGACGRVEEAVDVLLSRYPDVKVVPIITTCSTEIIGDDVDGVIKKLNEGLLKEKFPDREVHLIAMHTPSFVGSMISGYDVAVRDVVRHFAKREAPNDKINLLTGWVNPGDVKELKHLLGEMDIEANVLFEIESFDSPILPDGSAVSHGNTTIEDLIDTGNARATFALNRYEGTKAAEYLQKKFEIPAIIGPTPIGIRNTDIFLQNLKKATGKPIPQSLAHERGVAIDALADLTHMFLAEKRVAIYGAPDLVIGLAEFCLDLEMKPVLLLLGDDNSKYVDDPRIKALQENVDYGMEIVTNADFWELENRIKNEGLELDLILGHSKGRFISIDYNIPMLRVGFPTYDRAGLFRYPTVGYGGAIWLAEQMANTLFADMEHKKNKEWVLNVW

SEQ ID NO: 185 - protein, 339 aa, Artificial Sequence
  MTP-FA?51::HA::AnfH polypeptide
MAMAVFRREGRRLLPSIAARPIAAIRSPLSSDQEEGLLGVRSISTQVVRNRGGYPYDVPDYAGGMTRKVAIYGKGGIGKSTTTQNTAAALAYFHDKKVFIHGCDPKADSTRLILGGKPQETLMDMLRDKGAEKITNDDVIKKGFLDIQCVESGGPEPGVGCAGRGVITAIDLMEENGAYTDDLDFVFFDVLGDVVCGGFAMPIRDGKAQEVYIVASGEMMAIYAANNICKGLVKYAKQSGVRLGGIICNSRKVDGEREFLEEFTAAIGTKMIHFVPRDNIVQKAEFNKKTVTEFAPEENQAKEYGELARKIIENDEFVIPKPLTMDQLEDMVVKYGIAD

SEQ ID NO: 186 - protein, 196 aa, Artificial Sequence
  MTP-FA?51::HA::AnfG polypeptide
MAMAVFRREGRRLLPSIAARPIAAIRSPLSSDQEEGLLGVRSISTQVVRNRGGYPYDVPDYAGGMSTASAAAVVKQKVEAPVHPMDARIDELTDYIMKNCLWQFHSRSWDRERQNAEILKKTKELLCGEPVDLSTSHDRCYWVDAVCLADDYREHYPWINSMSKEEIGSLMQGLKDRMDYLTITGSLNEELSDKHY

SEQ ID NO: 187 - protein, 474 aa, Artificial Sequence
  HA::AnfK polypeptide
MYPYDVPDYAGGMTCEVKEKGRVGTINPIFTCQPAGAQFVSIGIKDCIGIVHGGQGCVMFVRLIFSQHYKESFELASSSLHEDGAVFGACGRVEEAVDVLLSRYPDVKVVPIITTCSTEIIGDDVDGVIKKLNEGLLKEKFPDREVHLIAMHTPSFVGSMISGYDVAVRDVVRHFAKREAPNDKINLLTGWVNPGDVKELKHLLGEMDIEANVLFEIESFDSPILPDGSAVSHGNTTIEDLIDTGNARATFALNRYEGTKAAEYLQKKFEIPAIIGPTPIGIRNTDIFLQNLKKATGKPIPQSLAHERGVAIDALADLTHMFLAEKRVAIYGAPDLVIGLAEFCLDLEMKPVLLLLGDDNSKYVDDPRIKALQENVDYGMEIVTNADFWELENRIKNEGLELDLILGHSKGRFISIDYNIPMLRVGFPTYDRAGLFRYPTVGYGGAIWLAEQMANTLFADMEHKKNKEWVLNVW

SEQ ID NO: 188 - protein, 287 aa, Artificial Sequence
  HA::AnfH polypeptide
MYPYDVPDYAGGMTRKVAIYGKGGIGKSTTTQNTAAALAYFHDKKVFIHGCDPKADSTRLILGGKPQETLMDMLRDKGAEKITNDDVIKKGFLDIQCVESGGPEPGVGCAGRGVITAIDLMEENGAYTDDLDFVFFDVLGDVVCGGFAMPIRDGKAQEVYIVASGEMMAIYAANNICKGLVKYAKQSGVRLGGIICNSRKVDGEREFLEEFTAAIGTKMIHFVPRDNIVQKAEFNKKTVTEFAPEENQAKEYGELARKIIENDEFVIPKPLTMDQLEDMVVKYGIAD

SEQ ID NO: 189 - protein, 144 aa, Artificial Sequence
  HA::AnfG polypeptide
MYPYDVPDYAGGMSTASAAAVVKQKVEAPVHPMDARIDELTDYIMKNCLWQFHSRSWDRERQNAEILKKTKELLCGEPVDLSTSHDRCYWVDAVCLADDYREHYPWINSMSKEEIGSLMQGLKDRMDYLTITGSLNEELSDKHY

SEQ ID NO: 190 - protein, 526 aa, Artificial Sequence
  mFA?51::HA::AnfK polypeptide
MAMAVFRREAAALLPSIAARPIAAAAAAASSDQEEGLLAAAAAAAAVVRNRGGYPYDVPDYAGGMTCEVKEKGRVGTINPIFTCQPAGAQFVSIGIKDCIGIVHGGQGCVMFVRLIFSQHYKESFELASSSLHEDGAVFGACGRVEEAVDVLLSRYPDVKVVPIITTCSTEIIGDDVDGVIKKLNEGLLKEKFPDREVHLIAMHTPSFVGSMISGYDVAVRDVVRHFAKREAPNDKINLLTGWVNPGDVKELKHLLGEMDIEANVLFEIESFDSPILPDGSAVSHGNTTIEDLIDTGNARATFALNRYEGTKAAEYLQKKFEIPAIIGPTPIGIRNTDIFLQNLKKATGKPIPQSLAHERGVAIDALADLTHMFLAEKRVAIYGAPDLVIGLAEFCLDLEMKPVLLLLGDDNSKYVDDPRIKALQENVDYGMEIVTNADFWELENRIKNEGLELDLILGHSKGRFISIDYNIPMLRVGFPTYDRAGLFRYPTVGYGGAIWLAEQMANTLFADMEHKKNKEWVLNVW

SEQ ID NO: 191 - protein, 339 aa, Artificial Sequence
  mFA?51::HA::AnfH polypeptide
MAMAVFRREAAALLPSIAARPIAAAAAAASSDQEEGLLAAAAAAAAVVRNRGGYPYDVPDYAGGMTRKVAIYGKGGIGKSTTTQNTAAALAYFHDKKVFIHGCDPKADSTRLILGGKPQETLMDMLRDKGAEKITNDDVIKKGFLDIQCVESGGPEPGVGCAGRGVITAIDLMEENGAYTDDLDFVFFDVLGDVVCGGFAMPIRDGKAQEVYIVASGEMMAIYAANNICKGLVKYAKQSGVRLGGIICNSRKVDGEREFLEEFTAAIGTKMIHFVPRDNIVQKAEFNKKTVTEFAPEENQAKEYGELARKIIENDEFVIPKPLTMDQLEDMVVKYGIAD

SEQ ID NO: 192 - protein, 196 aa, Artificial Sequence
  mFA?51::HA::AnfG polypeptide
MAMAVFRREAAALLPSIAARPIAAAAAAASSDQEEGLLAAAAAAAAVVRNRGGYPYDVPDYAGGMSTASAAAVVKQKVEAPVHPMDARIDELTDYIMKNCLWQFHSRSWDRERQNAEILKKTKELLCGEPVDLSTSHDRCYWVDAVCLADDYREHYPWINSMSKEEIGSLMQGLKDRMDYLTITGSLNEELSDKHY

SEQ ID NO: 193 - protein, 582 aa, Artificial Sequence
  mFA?51::HA::AnfD polypeptide
MAMAVFRREAAALLPSIAARPIAAAAAAASSDQEEGLLAAAAAAAAVVRNRGGYPYDVPDYAGGMPHHEFECSKVIPERKKHAVIKGKGETLADALPQGYLNTIPGSISERGCAYCGAKHVIGTPMKDVIHISHGPVGCTYDTWQTKRYISDNDNFQLKYTYATDVKEKHIVFGAEKLLKQNIIEAFKAFPQIKRMTIYQTCATALIGDDINAIAEEVMEEMPEVDIFVCNSPGFAGPSQSGGHHKINIAWINQKVGTVEPEITGDHVINYVGEYNIQGDQEVMVDYFKRMGIQVLSTFTGNGSYDGLRAMHRAHLNVLECARSAEYICNELRVRYGIPRLDIDGFGFKPLADSLRKIGMFFGIEDRAKAIIDEEVARWKPELDWYKERLMGKKVCLWPGGSKLWHWAHVIEEEMGLKVVSVYTKFGHQGDMEKGIARCGEGTLAIDDPNELEGLEALEMLKPDIILTGKRPGEVAKKVRVPYLNAHAYHNGPYKGFEGWVRFARDIYNAIYSPIHQLSGIDITKDNAPEWGNGFRTRQMLSDGNLSDAVRNSETLRQYTGGYDSVSKLREREYPAFERKVG

SEQ ID NO: 194 - protein, 582 aa, Artificial Sequence
  MTP-FA?51::HA::AnfD polypeptide
MAMAVFRREGRRLLPSIAARPIAAIRSPLSSDQEEGLLGVRSISTQVVRNRGGYPYDVPDYAGGMPHHEFECSKVIPERKKHAVIKGKGETLADALPQGYLNTIPGSISERGCAYCGAKHVIGTPMKDVIHISHGPVGCTYDTWQTKRYISDNDNFQLKYTYATDVKEKHIVFGAEKLLKQNIIEAFKAFPQIKRMTIYQTCATALIGDDINAIAEEVMEEMPEVDIFVCNSPGFAGPSQSGGHHKINIAWINQKVGTVEPEITGDHVINYVGEYNIQGDQEVMVDYFKRMGIQVLSTFTGNGSYDGLRAMHRAHLNVLECARSAEYICNELRVRYGIPRLDIDGFGFKPLADSLRKIGMFFGIEDRAKAIIDEEVARWKPELDWYKERLMGKKVCLWPGGSKLWHWAHVIEEEMGLKVVSVYTKFGHQGDMEKGIARCGEGTLAIDDPNELEGLEALEMLKPDIILTGKRPGEVAKKVRVPYLNAHAYHNGPYKGFEGWVRFARDIYNAIYSPIHQLSGIDITKDNAPEWGNGFRTRQMLSDGNLSDAVRNSETLRQYTGGYDSVSKLREREYPAFERKVG

SEQ ID NO: 195 - protein, 604 aa, Artificial Sequence
  MTP-FA?51::AnfD::Twin Strep polypeptide
MMAMAVFRREGRRLLPSIAARPIAAIRSPLSSDQEEGLLGVRSISTQVVRNRGGMPHHEFECSKVIPERKKHAVIKGKGETLADALPQGYLNTIPGSISERGCAYCGAKHVIGTPMKDVIHISHGPVGCTYDTWQTKRYISDNDNFQLKYTYATDVKEKHIVFGAEKLLKQNIIEAFKAFPQIKRMTIYQTCATALIGDDINAIAEEVMEEMPEVDIFVCNSPGFAGPSQSGGHHKINIAWINQKVGTVEPEITGDHVINYVGEYNIQGDQEVMVDYFKRMGIQVLSTFTGNGSYDGLRAMHRAHLNVLECARSAEYICNELRVRYGIPRLDIDGFGFKPLADSLRKIGMFFGIEDRAKAIIDEEVARWKPELDWYKERLMGKKVCLWPGGSKLWHWAHVIEEEMGLKVVSVYTKFGHQGDMEKGIARCGEGTLAIDDPNELEGLEALEMLKPDIILTGKRPGEVAKKVRVPYLNAHAYHNGPYKGFEGWVRFARDIYNAIYSPIHQLSGIDITKDNAPEWGNGFRTRQMLSDGNLSDAVRNSETLRQYTGGYDSVSKLREREYPAFERKVGGGSAWSHPQFEKGGGSGGGSGGSAWSHPQFEK

SEQ ID NO: 196 - protein, 523 aa, Artificial Sequence
  MTP-CoxIV::Twin Strep::AnfK polypeptide
MLSLRQSIRFFKPATRTLCSSRYLLQQKPSAWSHPQFEKGGGSGGGSGGSAWSHPQFEKGGMTCEVKEKGRVGTINPIFTCQPAGAQFVSIGIKDCIGIVHGGQGCVMFVRLIFSQHYKESFELASSSLHEDGAVFGACGRVEEAVDVLLSRYPDVKVVPIITTCSTEIIGDDVDGVIKKLNEGLLKEKFPDREVHLIAMHTPSFVGSMISGYDVAVRDVVRHFAKREAPNDKINLLTGWVNPGDVKELKHLLGEMDIEANVLFEIESFDSPILPDGSAVSHGNTTIEDLIDTGNARATFALNRYEGTKAAEYLQKKFEIPAIIGPTPIGIRNTDIFLQNLKKATGKPIPQSLAHERGVAIDALADLTHMFLAEKRVAIYGAPDLVIGLAEFCLDLEMKPVLLLLGDDNSKYVDDPRIKALQENVDYGMEIVTNADFWELENRIKNEGLELDLILGHSKGRFISIDYNIPMLRVGFPTYDRAGLFRYPTVGYGGAIWLAEQMANTLFADMEHKKNKEWVLNVW

SEQ ID NO: 197 - protein, 31 aa, Artificial Sequence
  peptide sequence
NSETLRQYTGGYDSVSKLREREYPAFERKVG

SEQ ID NO: 198 - protein, 16 aa, Artificial Sequence
  peptide sequence
GGGSGGGSGGGSGGGS

SEQ ID NO: 199 - protein, 984 aa, Artificial Sequence
  Amino acid sequence of AnfD::linker16::AnfK polypeptide used for modelling the structure (Example 20). Amino acids 1-509 correspond to the AnfD sequence (A. vinelandii) omitting the N-terminal methionine, amino acids 510-525 correspond to the
PHHEFECSKVIPERKKHAVIKGKGETLADALPQGYLNTIPGSISERGCAYCGAKHVIGTPMKDVIHISHGPVGCTYDTWQTKRYISDNDNFQLKYTYATDVKEKHIVFGAEKLLKQNIIEAFKAFPQIKRMTIYQTCATALIGDDINAIAEEVMEEMPEVDIFVCNSPGFAGPSQSGGHHKINIAWINQKVGTVEPEITGDHVINYVGEYNIQGDQEVMVDYFKRMGIQVLSTFTGNGSYDGLRAMHRAHLNVLECARSAEYICNELRVRYGIPRLDIDGFGFKPLADSLRKIGMFFGIEDRAKAIIDEEVARWKPELDWYKERLMGKKVCLWPGGSKLWHWAHVIEEEMGLKVVSVYTKFGHQGDMEKGIARCGEGTLAIDDPNELEGLEALEMLKPDIILTGKRPGEVAKKVRVPYLNAHAYHNGPYKGFEGWVRFARDIYNAIYSPIHQLSGIDITKDNAPEWGNGFRTRQMLSDGNSETLRQYTGGYDSVSKLREREYPAFERKVGGGGSGGGSGGGSGGGSVKEKGRVGTINPIFTCQPAGAQFVSIGIKDCIGIVHGGQGCVMFVRLIFSQHYKESFELASSSLHEDGAVFGACGRVEEAVDVLLSRYPDVKVVPIITTCSTEIIGDDVDGVIKKLNEGLLKEKFPDREVHLIAMHTPSFVGSMISGYDVAVRDVVRHFAKREAPNDKINLLTGWVNPGDVKELKHLLGEMDIEANVLFEIESFDSPILPDGSAVSHGNTTIEDLIDTGNARATFALNRYEGTKAAEYLQKKFEIPAIIGPTPIGIRNTDIFLQNLKKATGKPIPQSLAHERGVAIDALADLTHMFLAEKRVAIYGAPDLVIGLAEFCLDLEMKPVLLLLGDDNSKYVDDPRIKALQENVDYGMEIVTNADFWELENRIKNEGLELDLILGHSKGRFISIDYNIPMLRVGFPTYDRAGLFRYPTVGYGGAIWLAEQMANTLFADMEHKKNKEWVLNVW

SEQ ID NO: 200 - protein, 26 aa, Artificial Sequence
  Linker sequence
GGGGSGGGSYPYDVPDYAGGGSGGGS

SEQ ID NO: 201 - protein, 1004 aa, Artificial Sequence
  Amino acid sequence of AnfD::linker26(HA)::AnfK polypeptide used for modelling the structure. Amino acids 1-517 correspond to the AnfD sequence, amino acids 518-543 correspond to the 26-amino acid linker, and amino acids 544-1004 to AnfK
MPHHEFECSKVIPERKKHAVIKGKGETLADALPQGYLNTIPGSISERGCAYCGAKHVIGTPMKDVIHISHGPVGCTYDTWQTKRYISDNDNFQLKYTYATDVKEKHIVFGAEKLLKQNIIEAFKAFPQIKRMTIYQTCATALIGDDINAIAEEVMEEMPEVDIFVCNSPGFAGPSQSGGHHKINIAWINQKVGTVEPEITGDHVINYVGEYNIQGDQEVMVDYFKRMGIQVLSTFTGNGSYDGLRAMHRAHLNVLECARSAEYICNELRVRYGIPRLDIDGFGFKPLADSLRKIGMFFGIEDRAKAIIDEEVARWKPELDWYKERLMGKKVCLWPGGSKLWHWAHVIEEEMGLKVVSVYTKFGHQGDMEKGIARCGEGTLAIDDPNELEGLEALEMLKPDIILTGKRPGEVAKKVRVPYLNAHAYHNGPYKGFEGWVRFARDIYNAIYSPIHQLSGIDITKDNAPEWGNGFRTRQMLSDGNLSDAVRNSETLRQYTGGYDSVSKLREREYPAFERKVGGGGSGGGSYPYDVPDYAGGGSGGGSTCEVKEKGRVGTINPIFTCQPAGAQFVSIGIKDCIGIVHGGQGCVMFVRLIFSQHYKESFELASSSLHEDGAVFGACGRVEEAVDVLLSRYPDVKVVPIITTCSTEIIGDDVDGVIKKLNEGLLKEKFPDREVHLIAMHTPSFVGSMISGYDVAVRDVVRHFAKREAPNDKINLLTGWVNPGDVKELKHLLGEMDIEANVLFEIESFDSPILPDGSAVSHGNTTIEDLIDTGNARATFALNRYEGTKAAEYLQKKFEIPAIIGPTPIGIRNTDIFLQNLKKATGKPIPQSLAHERGVAIDALADLTHMFLAEKRVAIYGAPDLVIGLAEFCLDLEMKPVLLLLGDDNSKYVDDPRIKALQENVDYGMEIVTNADFWELENRIKNEGLELDLILGHSKGRFISIDYNIPMLRVGFPTYDRAGLFRYPTVGYGGAIWLAEQMANTLFADMEHKKNKEWVLNVW

SEQ ID NO: 202 - protein, 1068 aa, Artificial Sequence
  Amino acid sequence of the MTP-FA?51::AnfD::linker26(HA)::AnfK polypeptide encoded by SN272. Amino acids 1-64 correspond to the MTP-FA?51 sequence including the GG at its C-terminus, amino acids 65-581 correspond to the AnfD sequence (A. vinelandii),
MAMAVFRREGRRLLPSIAARPIAAIRSPLSSDQEEGLLGVRSISTQVVRNRGGYPYDVPDYAGGMPHHEFECSKVIPERKKHAVIKGKGETLADALPQGYLNTIPGSISERGCAYCGAKHVIGTPMKDVIHISHGPVGCTYDTWQTKRYISDNDNFQLKYTYATDVKEKHIVFGAEKLLKQNIIEAFKAFPQIKRMTIYQTCATALIGDDINAIAEEVMEEMPEVDIFVCNSPGFAGPSQSGGHHKINIAWINQKVGTVEPEITGDHVINYVGEYNIQGDQEVMVDYFKRMGIQVLSTFTGNGSYDGLRAMHRAHLNVLECARSAEYICNELRVRYGIPRLDIDGFGFKPLADSLRKIGMFFGIEDRAKAIIDEEVARWKPELDWYKERLMGKKVCLWPGGSKLWHWAHVIEEEMGLKVVSVYTKFGHQGDMEKGIARCGEGTLAIDDPNELEGLEALEMLKPDIILTGKRPGEVAKKVRVPYLNAHAYHNGPYKGFEGWVRFARDIYNAIYSPIHQLSGIDITKDNAPEWGNGFRTRQMLSDGNLSDAVRNSETLRQYTGGYDSVSKLREREYPAFERKVGGGGSGGGSYPYDVPDYAGGGSGGGSTCEVKEKGRVGTINPIFTCQPAGAQFVSIGIKDCIGIVHGGQGCVMFVRLIFSQHYKESFELASSSLHEDGAVFGACGRVEEAVDVLLSRYPDVKVVPIITTCSTEIIGDDVDGVIKKLNEGLLKEKFPDREVHLIAMHTPSFVGSMISGYDVAVRDVVRHFAKREAPNDKINLLTGWVNPGDVKELKHLLGEMDIEANVLFEIESFDSPILPDGSAVSHGNTTIEDLIDTGNARATFALNRYEGTKAAEYLQKKFEIPAIIGPTPIGIRNTDIFLQNLKKATGKPIPQSLAHERGVAIDALADLTHMFLAEKRVAIYGAPDLVIGLAEFCLDLEMKPVLLLLGDDNSKYVDDPRIKALQENVDYGMEIVTNADFWELENRIKNEGLELDLILGHSKGRFISIDYNIPMLRVGFPTYDRAGLFRYPTVGYGGAIWLAEQMANTLFADMEHKKNKEWVLNVW

SEQ ID NO: 203 - protein, 1065 aa, Artificial Sequence
  Amino acid sequence of the MTP-CoxIV::AnfD::linker26(HA)::AnfK polypeptide encoded by SN273. Amino acids 1-61 correspond to the MTP-CoxIV sequence including the GG at its C-terminus, amino acids 62-578 correspond to the AnfD sequence (A. vinelandii),
MLSLRQSIRFFKPATRTLCSSRYLLQQKPSAWSHPQFEKGGGSGGGSGGSAWSHPQFEKGGMPHHEFECSKVIPERKKHAVIKGKGETLADALPQGYLNTIPGSISERGCAYCGAKHVIGTPMKDVIHISHGPVGCTYDTWQTKRYISDNDNFQLKYTYATDVKEKHIVFGAEKLLKQNIIEAFKAFPQIKRMTIYQTCATALIGDDINAIAEEVMEEMPEVDIFVCNSPGFAGPSQSGGHHKINIAWINQKVGTVEPEITGDHVINYVGEYNIQGDQEVMVDYFKRMGIQVLSTFTGNGSYDGLRAMHRAHLNVLECARSAEYICNELRVRYGIPRLDIDGFGFKPLADSLRKIGMFFGIEDRAKAIIDEEVARWKPELDWYKERLMGKKVCLWPGGSKLWHWAHVIEEEMGLKVVSVYTKFGHQGDMEKGIARCGEGTLAIDDPNELEGLEALEMLKPDIILTGKRPGEVAKKVRVPYLNAHAYHNGPYKGFEGWVRFARDIYNAIYSPIHQLSGIDITKDNAPEWGNGFRTRQMLSDGNLSDAVRNSETLRQYTGGYDSVSKLREREYPAFERKVGGGGSGGGSYPYDVPDYAGGGSGGGSTCEVKEKGRVGTINPIFTCQPAGAQFVSIGIKDCIGIVHGGQGCVMFVRLIFSQHYKESFELASSSLHEDGAVFGACGRVEEAVDVLLSRYPDVKVVPIITTCSTEIIGDDVDGVIKKLNEGLLKEKFPDREVHLIAMHTPSFVGSMISGYDVAVRDVVRHFAKREAPNDKINLLTGWVNPGDVKELKHLLGEMDIEANVLFEIESFDSPILPDGSAVSHGNTTIEDLIDTGNARATFALNRYEGTKAAEYLQKKFEIPAIIGPTPIGIRNTDIFLQNLKKATGKPIPQSLAHERGVAIDALADLTHMFLAEKRVAIYGAPDLVIGLAEFCLDLEMKPVLLLLGDDNSKYVDDPRIKALQENVDYGMEIVTNADFWELENRIKNEGLELDLILGHSKGRFISIDYNIPMLRVGFPTYDRAGLFRYPTVGYGGAIWLAEQMANTLFADMEHKKNKEWVLNVW

SEQ ID NO: 204 - protein, 1068 aa, Artificial Sequence
  Amino acid sequence of the mFA?51::AnfD::linker26(HA)::AnfK polypeptide encoded by SN274. Amino acids 1-64 correspond to the mFA?51 sequence including the alanine substitutions that don′t allow for MPP-cleavage and the GG at its C-terminus, amino acids
MAMAVFRREAAALLPSIAARPIAAAAAAASSDQEEGLLAAAAAAAAVVRNRGGYPYDVPDYAGGMPHHEFECSKVIPERKKHAVIKGKGETLADALPQGYLNTIPGSISERGCAYCGAKHVIGTPMKDVIHISHGPVGCTYDTWQTKRYISDNDNFQLKYTYATDVKEKHIVFGAEKLLKQNIIEAFKAFPQIKRMTIYQTCATALIGDDINAIAEEVMEEMPEVDIFVCNSPGFAGPSQSGGHHKINIAWINQKVGTVEPEITGDHVINYVGEYNIQGDQEVMVDYFKRMGIQVLSTFTGNGSYDGLRAMHRAHLNVLECARSAEYICNELRVRYGIPRLDIDGFGFKPLADSLRKIGMFFGIEDRAKAIIDEEVARWKPELDWYKERLMGKKVCLWPGGSKLWHWAHVIEEEMGLKVVSVYTKFGHQGDMEKGIARCGEGTLAIDDPNELEGLEALEMLKPDIILTGKRPGEVAKKVRVPYLNAHAYHNGPYKGFEGWVRFARDIYNAIYSPIHQLSGIDITKDNAPEWGNGFRTRQMLSDGNLSDAVRNSETLRQYTGGYDSVSKLREREYPAFERKVGGGGSGGGSYPYDVPDYAGGGSGGGSTCEVKEKGRVGTINPIFTCQPAGAQFVSIGIKDCIGIVHGGQGCVMFVRLIFSQHYKESFELASSSLHEDGAVFGACGRVEEAVDVLLSRYPDVKVVPIITTCSTEIIGDDVDGVIKKLNEGLLKEKFPDREVHLIAMHTPSFVGSMISGYDVAVRDVVRHFAKREAPNDKINLLTGWVNPGDVKELKHLLGEMDIEANVLFEIESFDSPILPDGSAVSHGNTTIEDLIDTGNARATFALNRYEGTKAAEYLQKKFEIPAIIGPTPIGIRNTDIFLQNLKKATGKPIPQSLAHERGVAIDALADLTHMFLAEKRVAIYGAPDLVIGLAEFCLDLEMKPVLLLLGDDNSKYVDDPRIKALQENVDYGMEIVTNADFWELENRIKNEGLELDLILGHSKGRFISIDYNIPMLRVGFPTYDRAGLFRYPTVGYGGAIWLAEQMANTLFADMEHKKNKEWVLNVW

SEQ ID NO: 205 - protein, 1013 aa, Artificial Sequence
  Amino acid sequence of the HISx6::AnfD::linker26(HA)::AnfK polypeptide encoded by SN275, which does not have an MTP sequence and would be cytoplasmically located. Amino acids 1-9 correspond to the HISx6 sequence including the GG at its C-terminus, amino
MHHHHHHGGMPHHEFECSKVIPERKKHAVIKGKGETLADALPQGYLNTIPGSISERGCAYCGAKHVIGTPMKDVIHISHGPVGCTYDTWQTKRYISDNDNFQLKYTYATDVKEKHIVFGAEKLLKQNIIEAFKAFPQIKRMTIYQTCATALIGDDINAIAEEVMEEMPEVDIFVCNSPGFAGPSQSGGHHKINIAWINQKVGTVEPEITGDHVINYVGEYNIQGDQEVMVDYFKRMGIQVLSTFTGNGSYDGLRAMHRAHLNVLECARSAEYICNELRVRYGIPRLDIDGFGFKPLADSLRKIGMFFGIEDRAKAIIDEEVARWKPELDWYKERLMGKKVCLWPGGSKLWHWAHVIEEEMGLKVVSVYTKFGHQGDMEKGIARCGEGTLAIDDPNELEGLEALEMLKPDIILTGKRPGEVAKKVRVPYLNAHAYHNGPYKGFEGWVRFARDIYNAIYSPIHQLSGIDITKDNAPEWGNGFRTRQMLSDGNLSDAVRNSETLRQYTGGYDSVSKLREREYPAFERKVGGGGSGGGSYPYDVPDYAGGGSGGGSTCEVKEKGRVGTINPIFTCQPAGAQFVSIGIKDCIGIVHGGQGCVMFVRLIFSQHYKESFELASSSLHEDGAVFGACGRVEEAVDVLLSRYPDVKVVPIITTCSTEIIGDDVDGVIKKLNEGLLKEKFPDREVHLIAMHTPSFVGSMISGYDVAVRDVVRHFAKREAPNDKINLLTGWVNPGDVKELKHLLGEMDIEANVLFEIESFDSPILPDGSAVSHGNTTIEDLIDTGNARATFALNRYEGTKAAEYLQKKFEIPAIIGPTPIGIRNTDIFLQNLKKATGKPIPQSLAHERGVAIDALADLTHMFLAEKRVAIYGAPDLVIGLAEFCLDLEMKPVLLLLGDDNSKYVDDPRIKALQENVDYGMEIVTNADFWELENRIKNEGLELDLILGHSKGRFISIDYNIPMLRVGFPTYDRAGLFRYPTVGYGGAIWLAEQMANTLFADMEHKKNKEWVLNVW

SEQ ID NO: 206 - protein, 383 aa, Thermoanaerobacter brockii
MTLKKGKKVYIVDTTLRDGEQTAGVVFANNEKIRIAQMLDEIGIDQLEVGIPTMGGDEKETVTKIAKLGLNASIMAWNRAVVKDVQESLECGVDAVAISVSTSDIHIEHKLKKTRQWVLDNMTEAVKFAKKEGVYVSVNAEDASRTDMNFLIEFAKCAKQAGADRLRFCDTVGFLDPFKTYDMVKAIKEAVDIDIEMHTHNDFGMATANALAGMRAGANFIGVTVNGLGERAGNAALEEVVMALKHVYKIDLGIDTTRFREISEYVALASGRQLPAWKAIVGTNVFAHESGIHVDGALKNPHTYEIFNPDEVGLERQIVIGKHSGTAALINKFKEYGRVLTEEEANLLLPHVRKLAIQLKRPLFDKELMYLYEDVIKNREKAI

SEQ ID NO: 207 - protein, 378 aa, Thermincola potens
MMEKKITIVDTTLRDGEQTAGVVFANKEKVRIAQMLDELGVHQIEAGIPVMGGDEEKAVKDIVKLGLKASIMGWNRAVISDIEETLRCGVDAVAISISTSDIHIQHKLQTSREWVLENMVKAVEFAKKEGVYISVNAEDASRSDMEFLIQFARAAKEAGADRIRYCDTIGILDPFTTYENIQTLKKHVDIDIEMHTHNDFGMATANALAGIKAGASHVGVTVMGLGERAGNAALEEVVMALKHIWQIDLGFKTNMFRDLAEYVSLASGRELPAWKAIVGSNMFAHESGIHADGAIKNPITYEVFAPEEVGLERQIVIGKHSGSKALMMKFAEYGIHLSEADAAQLLPKIRSHAVALKRSLFDKELVYIYEEVFGKKPL

SEQ ID NO: 208 - protein, 440 aa, Saccharomyces cerevisiae
MSENNEFQSVTESTTAPTTSNPYGPNPADYLSNVKNFQLIDSTLREGEQFANAFFDTEKKIEIARALDDFGVDYIELTSPVASEQSRKDCEAICKLGLKAKILTHIRCHMDDARVAVETGVDGVDVVIGTSKFLRQYSHGKDMNYIAKSAVEVIEFVKSKGIEIRFSSEDSFRSDLVDLLNIYKTVDKIGVNRVGIADTVGCANPRQVYELIRTLKSVVSCDIECHFHNDTGCAIANAYTALEGGARLIDVSVLGIGERNGITPLGGLMARMIVAAPDYVRSKYKLHKIRDIENLVADAVEVNIPFNNPITGFCAFTHKAGIHAKAILANPSTYEILDPHDFGMKRYIHFANRLTGWNAIKSRVDQLNLNLTDDQIKEVTAKIKKLGDVRPLNIDDVDSIIKDFHAELSTPLLKPVNKGTDDDNIDISNGHVSKKAKVTK

SEQ ID NO: 209 - protein, 376 aa, Nodularia spumigena
MNEILINDTTLRDGEQAAGVAFTFEEKVAIAQFLDAIGVPELEVGIPAMGEAETHAILAISDLGLQASLLGWNRAVLSDIKASITCGLKRVHIAIPVSGIQIAAKFHGQWRVSLQRLKDCISFAVDQGLWVAVGGEDSSRADPNFLLDVALNAQEWGASRFRFCDTVGVLDPFSTYAKVKQLVSALSIPLEIHTHNDFGLATANALAGIKAGATSVNTTVNGVGERAGNAALEEVVMSIKRIYGINLGIDTRRLLELSQLVASASNCHVPPWKAIVGENTFAHESGIHAHGVLQNPLTYEPFAPEEVGWERRLVVGKHSGRHLVTSLLQQNDIFLNPEETQSVLDAVRQQSVKQKRNLTVEELLNLVREQRYSHAT

SEQ ID NO: 210 - protein, 376 aa, Methanosarcina acetivorans
MKADIKVSIVDQTINEVVRLGVNNPADVRFMLSVLKKYSFDAADVSLNNLEKNMVEFEADEFSESMRCRVKCSGQEIFRAKKLGFSKIVINTSLNPLTPIQDMLEPVLQMACSNDQEIYLSIDNALEFSIRDVETIYPLIPKYGIKRLILGDRSGKADPFTTYDKLGFLGNTIQCPVEYVGYNDYGTATANTLSALRAGIEYVATAVSGIGIPGVAAMEEVLMAARHLWKNELVPDGYSIAADCENILYRAGIMLPGEKAIIGKNVFAHESGIHVDGVLKNPNLYEAIKPEEVGLRRLLVIGKHSGTASLVQKLRHLGLSLSPEKAAALLEKVRNTAILQKKPLTDLQLKTLYDLQMESVKDPNIHLSGKGEMPCD

SEQ ID NO: 211 - protein, 376 aa, Chlorobaculum tepidum
MIRKPWIIDTTLRDGEQAPGVVFSPHEKKRIAAMLAETGVDEIEVGYPAISAAERKVIREIVAMKLPVRLTSWSRANMADIELAAECGTDAVHISFPASRLYLELIHKKDDWIQEQLHALVSKARERFDFVSVGGQDATRSSTDFLQRFMLDAEAAGAKRFRIADTVGIATPVSVMALGAALRQSSSLPLEFHAHNDLGMATANAFTALNEGFEAVSVSVTGLGERAGNAALEELAMALALNGDFDTHLDTSMLSRLCDAVATASGRAIQEQKPVVGRSAFQHESGIHCAALLQDPLSYQPFLPSRVGRSDFEIVIGKHSGTAAIIAHFNRRGITISKKEARELLDLIRSQSDRLKRALRTDEIDALREQNSVKHA

SEQ ID NO: 212 - protein, 511 aa, Methanocaldococcus infernus
MILYKEENEIIKEALKGLKLPDRVYIFDTTLRDGEQTPGVSLTVDEKVEIAINLDKLGVDIIEAGFPISSSGEYEAVKKIASLNLDAEICALARAVKEDIDRAIDCGVDRIHTFIATSPLHRKYKLKMSKEEIVEKAVNAIEYIKEHGIKVEFSAEDATRTEIDYLKEVYKKAVEAGADIINVPDTVGVMIPRATYYLISELRKEIDNISVHCHNDFGLAVANSLAAVEAGAIQCHVTVNGLGERGGNAALEEVVTSLHFIYGIKTKVKTEELYNISKLVEKLTEVKVQPNKAVVGDNAFAHESGIHAHGVLAHALTYEPIPPELVGQRRRIILGKHTGTHAIEAKLKELGYTNINKEQFKEIVKRIKSLGDKGKRVTDKDVEAIVEDVIGRVSKRERVVDLEQIAVMTGNKVIPTASVALKINDNLIKTSAIGVGPVDAAVKAIQKAIGEKIKIKEYHIDAITGGTDALAEVVVTLEGYGKEITTKAAREDIVRASVEAVIDGINKILKK

SEQ ID NO: 213 - protein, 490 aa, Methanocaldococcus infernus
MKVRVFDTTLRDGEQTPGVSLTPSDKLEIAKALDELGVDVIEAGSAITSKGEREGIKLITRENLNAEICSFVRPLTVDIDAAIECEVDSIHLVVPSSPIHIKYKLKKSEDEVLDLAVNAIEYAKDHGLIVELSAEDATRAELNFLIKLFKAGENLADRVCVCDTVGVLTPQKSEELFKKITSEIKLPVSVHCHNDFGMATANTCSAILGGAVQCHVTVNGIGERAGNAALEEVVTALKFLYNIETNIKLEKLYEVSRLVARLMKLPVPPNKAIVGDNAFAHEAGIHVDGLIKNTKTYEPISPEVVGNKRRIILGKHSGRKALIYKLKLMGIEASEEQINKIYEKIKELGDLGKYVSDADLMAIVKDVLGKDLEEKIELDELTVVSGNKITPIASVKLHYKGEDRLLIETAYGVGPVDAAINAVRKAISGVADIKLEEYKVEAIGGGTDAIIEVTVKLRKGVNTVEVKKADSDIIRASVNAVMEGINLLLQ

SEQ ID NO: 214 - protein, 396 aa, Methanocaldococcus infernus
MDLLYENTWKAPSPYNPKLKLKDIYIYDTTLRDGEQTPGVCFTKEQKLEIARALDELGVSQIEAGFPIVSKREAEIVKAIASENLNADILALSRIRKEDINKAIDCDVDGIITFIATSPLHIKCKFKGKRLEDFFDTIVECIEYAKSHGLFVAFSAEDGTRTPLEDLIRVHKLAEEAGADRVHVADTAGTATPQAMEFICKALTCSLNKAHVGVHCHNDFGLAVINSIYGLIGGAKAVSTTVNGIGERAGNTSLEELIMSLIVLYDVDLKLNLEVLPKLCRMVEEYSGIKNPKNKPIVGELVFSHESGIHVDAVIENPLTYEPFLPEKIGLKRNIILGKHSGKRAVKYKLKLLGVEVEDKLLDKIVERVKELREKGEKIDDEKLLEIVEEIKRIKD

SEQ ID NO: 215 - protein, 540 aa, Lotus japonicus
MASKNSIIRNRPEYIPNHIPNPTYVRILDTTLRDGEQSPGAAMTCVQKLETARQLAKLGVDIIEAGFPCASKQDFMAVKMIAEEVGNCVDGNGYVPVITGVSRCNEKDIATAWEALKHAKRPRLRTFIATSPIHMEYKLRKSKDQVLETARNMVKFARSLGCTDIQFGAEDAARSDKEFLYQIFGEVIKAGATTLTIPDTVGIAMPFEYGKLIADIKANTPGIENAIMATHCHNDLGLATANTIEGARYGARQLEVTINGIGERAGNASFEEVVMALTCRGIDILGGLHTGINTRHILKTSKMVEKYSGLHLQPHKALVGANAFLHESGIHQDGMLKHRGTYEIISPEDIGLVRSVGDTIVLGKLSGRQALRNRLEELGYKLKDTEVEGVFWQFKAVAEKKKRITDTDLRALVSNEAFNEQPIWKLGDLQVTCGTVGFSTATVKLFSIDGSMHVACSIGTGPVDSAYKAINHIVKEPAKLVKYTLGAITEGIDATATTSVEISRGDTNHPVFSGTGGGTDVVVSSVDAYLSALNNMLRFY

SEQ ID NO: 216 - protein, 518 aa, Azotobacter vinelandii
MPHHEFECSKVIPERKKHAVIKGKGETLADALPQGYLNTIPGSISERGCAYCGAKHVIGTPMKDVIHISHGPVGCTYDTWQTKRYISDNDNFQLKYTYATDVKEKHIVFGAEKLLKQNIIEAFKAFPQIKRMTIYQTCATALIGDDINAIAEEVMEEMPEVDIFVCNSPGFAGPSQSGGHHKINIAWINQKVGTVEPEITGDHVINYVGEYNIQGDQEVMVDYFKRMGIQVLSTFTGNGSYDGLRAMHRAHLNVLECARSAEYICNELRVRYGIPRLDIDGFGFKPLADSLRKIGMFFGIEDRAKAIIDEEVARWKPELDWYKERLMGKKVCLWPGGSKLWHWAHVIEEEMGLKVVSVYTKFGHQGDMEKGIARCGEGTLAIDDPNELEGLEALEMLKPDIILTGKRPGEVAKKVRVPYLNAHAYHNGPYKGFEGWVRFARDIYNAIYSPIHQLSGIDITKDNAPEWGNGFRTRQMLSDGNLSDAVRNSETLRQYTGGYDSVSKLREREYPAFERKVG

SEQ ID NO: 217 - protein, 462 aa, Azotobacter vinelandii
MTCEVKEKGRVGTINPIFTCQPAGAQFVSIGIKDCIGIVHGGQGCVMFVRLIFSQHYKESFELASSSLHEDGAVFGACGRVEEAVDVLLSRYPDVKVVPIITTCSTEIIGDDVDGVIKKLNEGLLKEKFPDREVHLIAMHTPSFVGSMISGYDVAVRDVVRHFAKREAPNDKINLLTGWVNPGDVKELKHLLGEMDIEANVLFEIESFDSPILPDGSAVSHGNTTIEDLIDTGNARATFALNRYEGTKAAEYLQKKFEIPAIIGPTPIGIRNTDIFLQNLKKATGKPIPQSLAHERGVAIDALADLTHMFLAEKRVAIYGAPDLVIGLAEFCLDLEMKPVLLLLGDDNSKYVDDPRIKALQENVDYGMEIVTNADFWELENRIKNEGLELDLILGHSKGRFISIDYNIPMLRVGFPTYDRAGLFRYPTVGYGGAIWLAEQMANTLFADMEHKKNKEWVLNVW

SEQ ID NO: 218 - protein, 275 aa, Azotobacter vinelandii
MTRKVAIYGKGGIGKSTTTQNTAAALAYFHDKKVFIHGCDPKADSTRLILGGKPQETLMDMLRDKGAEKITNDDVIKKGFLDIQCVESGGPEPGVGCAGRGVITAIDLMEENGAYTDDLDFVFFDVLGDVVCGGFAMPIRDGKAQEVYIVASGEMMAIYAANNICKGLVKYAKQSGVRLGGIICNSRKVDGEREFLEEFTAAIGTKMIHFVPRDNIVQKAEFNKKTVTEFAPEENQAKEYGELARKIIENDEFVIPKPLTMDQLEDMVVKYGIAD

SEQ ID NO: 219 - protein, 132 aa, Azotobacter vinelandii
MSTASAAAVVKQKVEAPVHPMDARIDELTDYIMKNCLWQFHSRSWDRERQNAEILKKTKELLCGEPVDLSTSHDRCYWVDAVCLADDYREHYPWINSMSKEEIGSLMQGLKDRMDYLTITGSLNEELSDKHY

SEQ ID NO: 220 - protein, 4 aa, Artificial Sequence
  Peptide
RRNF

SEQ ID NO: 221 - protein, 606 aa, Nicotiana benthamiana
MASITTNHTFSRNLNFSFHPQNPLIQTQALFKFKPSIPNCSPIIRCAIRRRPEYTPSHIPDPKYIRIFDTTLRDGEQSPGATMTTKEKLDVARQLAKLGVDIIEAGFPASSEADLEAVKLIAKEVGNGVYEEGHVPVICGLARCNKRDIDKAWEAVKYAKKPRIHTFIATSEIHMKFKLKMSRDEVVEKARSMVAYARSIGCEDVEFSPEDAGRSDPEFLYHILGEVIKAGATTLNIPDTVGYTVPSEFGKLIADIKANTPGIGDVIISTHCQNDLGLSTANTLAGACAGARQVEVTINGIGERAGNASLEEVVMALKCRGEQVLGGLYTGINTQHILMSSKMVEEYTGLHVQPHKAIVGANAFAHESGIHQDGMLKHKDTYEIISPEDIGLNRANEAGIVLGKLSGRHALKSKMLELGYDIEGKELEDLFWRFKSVAEKKKKVTDDDIIALMSDEVFQPQVVWQLADVQITCGSLGLSTATVKLIDSDGQDHVACSVGTGPVDAAYKAVDLIVKVPITLLEYSMNAVTEGIDSIASTRVVIREEDDHAITNGSIGLTLHRTFSGTGADMDVVISSVRAYIGALNKMLSFGKLVSRCNNPEGSVVV

SEQ ID NO: 222 - protein, 470 aa, Nicotiana benthamiana
MASIYANPTTSLNTSLSSYSKNIFLRSVFKFMPSITKRCHCPYTSTAVRCTNVRRPRPNYRPGRFSHPNYVGIFDTTLRDGEQAPGAAMTITEKLDIARQLAKLGVDVIEAGFPAASDADFELVKLVAKEVGNNVDEEGYVPVICAIGRTTKKDIDRTWEALKYAKKPMISLFIATSDIHMKYKLKMSKEVVVEKARSMVAYAKTLCEDVRFCVEDGARSDRKFLYYILGEGIKVGATAICVADTVGSSLPTEFGRLIADIKANTPGIEDVIISVHCHNDLGLATANTLAGACAGARLVDVTVNGIGERAGNGSLEEIVMALKYRGEEVLGGLYSGINTKHIIATSKMVEEYCGLKLQPHKPIVGANAFSHESGIHQDGVLKKRETYEFVSPEDVGFQRVTGHGIILGKLSGRHALKSKMFELGYEFEGKELDDIFRRFKSVAEKKKKITEEDLRALVSDKVCSLKLLDA

SEQ ID NO: 223 - protein, 644 aa, Mycobacterium tuberculosis
MATSESPDAYTESFGAHTIVKPAGPPRVGQPSWNPQRASSMPVNRYRPFAEEVEPIRLRNRTWPDRVIDRAPLWCAVDLRDGNQALIDPMSPARKRRMFDLLVRMGYKEIEVGFPSASQTDFDFVREIIEQGAIPDDVTIQVLTQCRPELIERTFQACSGAPRAIVHFYNSTSILQRRVVFRANRAEVQAIATDGARKCVEQAAKYPGTQWRFEYSPESYTGTELEYAKQVCDAVGEVIAPTPERPIIFNLPATVEMTTPNVYADSIEWMSRNLANRESVILSLHPHNDRGTAVAAAELGFAAGADRIEGCLFGNGERTGNVCLVTLGLNLFSRGVDPQIDFSNIDEIRRTVEYCNQLPVHERHPYGGDLVYTAFSGSHQDAINKGLDAMKLDADAADCDVDDMLWQVPYLPIDPRDVGRTYEAVIRVNSQSGKGGVAYIMKTDHGLSLPRRLQIEFSQVIQKIAEGTAGEGGEVSPKEMWDAFAEEYLAPVRPLERIRQHVDAADDDGGTTSITATVKINGVETEISGSGNGPLAAFVHALADVGFDVAVLDYYEHAMSAGDDAQAAAYVEASVTIASPAQPGEAGRHASDPVTIASPAQPGEAGRHASDPVTSKTVWGVGIAPSITTASLRAVVSAVNRAAR

SEQ ID NO: 224 - protein, 290 aa, Azotobacter vinelandii
MAMRQCAIYGKGGIGKSTTTQNLVAALAEMGKKVMIVGCDPKADSTRLILHSKAQNTIMEMAAEAGTVEDLELEDVLKAGYGGVKCVESGGPEPGVGCAGRGVITAINFLEEEGAYEDDLDFVFYDVLGDVVCGGFAMPIRENKAQEIYIVCSGEMMAMYAANNISKGIVKYANSGSVRLGGLICNSRNTDREDELIIALANKLGTQMIHFVPRDNVVQRAEIRRMTVIEYDPKAKQADEYRALARKVVDNKLLVIPNPITMDELEELLMEFGIMEVEDESIVGKTAEEV

SEQ ID NO: 225 - protein, 15 aa, Artificial Sequence
  Peptide
  X (12)..(12): any amino acid
YGKGGIGKSTTXQNT

SEQ ID NO: 226 - protein, 9 aa, Artificial Sequence
  Peptide
IHGCDPKAD

SEQ ID NO: 227 - protein, 17 aa, Artificial Sequence
  Peptide
CVESGGPEPGVGCAGRG

SEQ ID NO: 228 - protein, 14 aa, Artificial Sequence
  Peptide
DVLGDVVCGGFAMP

SEQ ID NO: 229 - protein, 15 aa, Artificial Sequence
  Peptide
  X (9)..(9): any amino acid
VASGEMMAXYAANNI

SEQ ID NO: 230 - protein, 5 aa, Artificial Sequence
  Peptide
QSGVR

SEQ ID NO: 231 - protein, 7 aa, Artificial Sequence
  Peptide
  X (5)..(5): any amino acid
CNSRXVD

SEQ ID NO: 232 - protein, 92 aa, Azotobacter vinelandii
MALKIVESCVNCWACVDVCPSEAISLAGPHFEISASKCTECDGDYAEKQCASICPVEGAILLADGTPANPPGSLTGIPPERLAEAMREIQAR

SEQ ID NO: 233 - protein, 157 aa, Artificial Sequence
  Synthetic fusion polypeptide
MMAMAVFRREGRRLLPSIAARPIAAIRSPLSSDQEEGLLGVRSISTQVVRNRGGALKIVESCVNCWACVDVCPSEAISLAGPHFEISASKCTECDGDYAEKQCASICPVEGAILLADGTPANPPGSLTGIPPERLAEAMREIQARAGGYPYDVPDYA

SEQ ID NO: 234 - protein, 156 aa, Artificial Sequence
  Synthetic fusion polypeptide
MAMAVFRREGRRLLPSIAARPIAAIRSPLSSDQEEGLLGVRSISTQVVRNRGGYPYDVPDYAGGALKIVESCVNCWACVDVCPSEAISLAGPHFEISASKCTECDGDYAEKQCASICPVEGAILLADGTPANPPGSLTGIPPERLAEAMREIQARA

SEQ ID NO: 235 - protein, 156 aa, Artificial Sequence
  Synthetic fusion polypeptide
MAMAVFRREAAALLPSIAARPIAAAAAAASSDQEEGLLAAAAAAAAVVRNRGGYPYDVPDYAGGALKIVESCVNCWACVDVCPSEAISLAGPHFEISASKCTECDGDYAEKQCASICPVEGAILLADGTPANPPGSLTGIPPERLAEAMREIQARA

SEQ ID NO: 236 - protein, 104 aa, Artificial Sequence
  Synthetic fusion polypeptide
MYPYDVPDYAGGALKIVESCVNCWACVDVCPSEAISLAGPHFEISASKCTECDGDYAEKQCASICPVEGAILLADGTPANPPGSLTGIPPERLAEAMREIQARA

SEQ ID NO: 237 - protein, 448 aa, Artificial Sequence
  Synthetic fusion polypeptide
MAMAVFRREGRRLLPSIAARPIAAIRSPLSSDQEEGLLGVRSISTQVVRNRGGYPYDVPDYAGGMASVIIDDTTLRDGEQSAGVAFNADEKIAIARALAELGVPELEIGIPSMGEEEREVMHAIAGLGLSSRLLAWCRLCDVDLAAARSTGVTMVDLSLPVSDLMLHHKLNRDRDWALREVARLVGEARMAGLEVCLGCEDASRADLEFVVQVGEVAQAAGARRLRFADTVGVMEPFGMLDRFRFLSRRLDMELEVHAHDDFGLATANTLAAVMGGATHINTTVNGLGERAGNAALEECVLALKNLHGIDTGIDTRGIPAISALVERASGRQVAWQKSVVGAGVFTHEAGIHVDGLLKHRRNYEGLNPDELGRSHSLVLGKHSGAHMVRNTYRDLGIELADWQSQALLGRIRAFSTRTKRSPQPAELQDFYRQLCEQGNPELAAGGMA

SEQ ID NO: 238 - protein, 243 aa, Azotobacter vinelandii
MVTPVNMSRETALRIALAARALPGTTVGQLLEILHQRIEGPLTEESLQGVSVTDLKIGLAGSEEDVDMLDTPMSALKDAVRILWGEAEVDSLPQPVKLERVPEGSIRVAIASNNGEQLDGHFGSCLRFLVYQVSAKDASLVDIRSTLDVALAEDKNAWRVEQIQDCQVLYVVSIGGPAAAKVVRAGIHPLKKPKGCAAQEAIAELQTVMAGSPPPWLAKLVGVSAEERVRFSVSDDEDEAARA

SEQ ID NO: 239 - protein, 4 aa, Artificial Sequence
  C-terminal amino acid sequence
DLIR

SEQ ID NO: 240 - protein, 4 aa, Artificial Sequence
  C-terminal amino acid sequence
DVVR

SEQ ID NO: 241 - protein, 4 aa, Artificial Sequence
  C-terminal amino acid sequence
DIIR

SEQ ID NO: 242 - protein, 4 aa, Artificial Sequence
  C-terminal amino acid sequence
DLTR

SEQ ID NO: 243 - protein, 4 aa, Artificial Sequence
  C-terminal amino acid sequence
INVW

SEQ ID NO: 244 - protein, 4 aa, Artificial Sequence
  C-terminal amino acid sequence
LNVW

SEQ ID NO: 245 - protein, 4 aa, Artificial Sequence
  C-terminal amino acid sequence
LNTW

SEQ ID NO: 246 - protein, 4 aa, Artificial Sequence
  C-terminal amino acid sequence
LNMW

SEQ ID NO: 247 - protein, 4 aa, Artificial Sequence
  C-terminal amino acid sequence
LAMW

SEQ ID NO: 248 - protein, 4 aa, Artificial Sequence
  C-terminal amino acid sequence
LSVW

(19) United States
(12) Patent Application Publication
(10) Pub. No.: US 2024/0117326 A1
(43) Pub. Date: Apr. 11, 2024

(54) EXPRESSION OF NITROGENASE POLYPEPTIDES IN PLANT CELLS

(71) Applicant: Commonwealth Scientific and Industrial Research Organisation, Acton (AU)

(72) Inventors: Ema Jayne JOHNSTON, Canberra (AU); Robert Silas ALLEN, Downer (AU); Christina Maria GREGG, Canberra (AU); Shoko OKADA, Coombs (AU); Amratha MENON, Canberra (AU); Andrew Charles WARDEN, Ngunnawal (AU); Matthew Craig TAYLOR, Chifley (AU); Craig Christopher WOOD, Dickson (AU)

(73) Assignee: Commonwealth Scientific and Industrial Research Organisation, Acton (AU)

(21) Appl. No.: 17/767,838

(22) PCT Filed: Oct. 9, 2020
(86) PCT No.: PCT/AU2020/051089

§ 371 Date: Apr. 8, 2022

(30) Foreign Application Priority Data
Oct. 10, 2019 (AU) 2019903818
Mar. 5, 2020 (AU) 2020900689

(51) Int. Cl. C12N 9/02; C07K 14/415; C12N 9/10; C12N 9/24; C12N 15/62; C12N 15/82

(52) U.S. Cl. CPC C12N 9/0095; C07K 14/415; C12N 9/1025; C12N 9/13; C12N 9/2402; C12N 15/62; C12N 15/8243; C12Y 118/06001; C12Y 203/03014; C12Y 208/01007; C07K 2319/07; C07K 2319/50

(57) ABSTRACT

The present invention relates to methods and means for producing nitrogenase polypeptides in the mitochondria of plant cells.

### FIELD OF THE INVENTION

The present invention relates to methods and means for producing nitrogenase polypeptides in the mitochondria of plant cells.

### BACKGROUND OF THE INVENTION

Diazotrophic bacteria produce ammonia from N₂ gas via biological nitrogen fixation (BNF), catalysed by the enzyme complex, nitrogenase. Yet the demands of modern agriculture γr outstrip this source of fixed nitrogen, and consequently industrially-produced nitrogenous fertiliser is used extensively in agriculture (Smil, 2002). However, both fertiliser production and application are causes of pollution (Good and Beatty, 2011) and considered unsustainable (Rockstrom et al., 2009). The majority of fertilizer applied worldwide is not taken up by crops (Cui et al., 2013; de Bruijn, 2015), leading to fertilizer runoff, promotion of weeds and eutrophication of waterways (Good and Beatty, 2011). Resultant algal blooms reduce oxygen levels, causing environmental damage locally and offshore throughout coral reefs (De'ath et al., 2012; Glibert et al., 2014; Sutton et al., 2008). Furthermore although over fertilization is a problem in many developed countries, in certain regions it's availability limits crop yields (Mueller et al., 2012). The production of fertilizer itself requires substantial energy inputs, and costs an estimated $100 USD billon/γr.

Clearly strategies to reduce industrially-produced nitrogenous dependence are required. To this end, the notion of engineering plants capable of biological nitrogen fixation has long attracted considerable interest (Merrick and Dixon, 1984), and has been the focus of recent reviews (de Bruijn, 2015; Oldroyd and Dixon, 2014). Potential approaches include i) extending the symbiotic relationship of diazotrophs from legumes to cereals (Santi et al., 2013), ii) re-engineering endosymbiotic microorganisms to be capable of nitrogen fixation (Geddes et al., 2015), and iii) genetic engineering of nitrogenase into plant cells (Curatti and Rubio, 2014). All of these approaches are ambitious and speculative due to the technical difficulty.

Nitrogenase, the enzyme complex capable of biological nitrogen fixation in diazotrophic bacteria, requires a multigene assembly pathway for its biosynthesis and function, reviewed extensively (Hu and Ribbe, 2013; Rubio and Ludden, 2008; Seefeldt et al., 2009). The components of the canonical iron-molybdenum nitrogenase include the catalytic proteins designated NifD and NifK and the electron donor NifH. About 12 other proteins are involved in nitrogenase assembly in diazotrophic bacteria including in the maturation, scaffolding and co-factor insertion of the complex, specifically NifM, NifS, NifU, NifE, NifN, NifX, NifV, NifJ, NifY, NifF, NifZ and NifQ. Genetic lesions, complementation assays between diazotrophs to non-diazotrophic prokaryotes and phylogenetic analyses (Dos Santos et al., 2012; Temme et al., 2012; Wang et al., 2013) have led to a subset of Nif proteins (NifD, NifK, NifB, NifE and NifN) being considered as the core components, whilst others are thought to be required for optimised activity and are considered auxiliary. Specific biochemical conditions are also required for nitrogenase assembly and function. Foremost among these, nitrogenase is extremely oxygen sensitive (Robson and Postgate, 1980). Furthermore large amounts of ATP, reductant, readily available Fe, Mo, S-adenosylmethionine and homocitrate are required for biosynthesis and function of the metalloprotein catalytic centre (Hu and Ribbe, 2013; Rubio and Ludden, 2008). All of these factors contribute to the technical difficulty of producing a functional nitrogenase complex in plant cells.

### SUMMARY OF THE INVENTION

The present inventors have determined the importance of expressing a NifD that is resistant to secondary cleavage/degradation in plant cells, in view of the observed difficulty in producing functional NifD in plant cells.

Thus, in an aspect, the present invention provides a plant cell comprising an exogenous polynucleotide which encodes a NifD polypeptide (ND) which is resistant to protease cleavage at a site within an amino acid sequence corresponding to amino acids 97-100 of SEQ ID NO:18.

In a related aspect, the present invention provides a plant cell comprising an exogenous polynucleotide which encodes a NifD polypeptide (ND) which comprises an amino acid sequence other than RRNY (SEQ ID NO:101) at positions corresponding to amino acids 97-100 of SEQ ID NO:18. In a preferred embodiment, the ND is more resistant to protease cleavage at a site within an amino acid sequence corresponding to amino acids 97-100 of SEQ ID NO:18 than a corresponding ND which has the amino acid sequence RRNY (SEQ ID NO:101) at positions corresponding to amino acids 97-100 of SEQ ID NO:18.

In an embodiment of the above aspects, the ND comprises a mitochondrial targeting peptide (MTP), preferably wherein the MTP is at the N-terminal end of the ND.

In a further embodiment, the ND is capable of being cleaved within the MTP, or immediately after the MTP, to yield a processed NifD polypeptide (CND) when the exogenous polynucleotide is expressed in the plant cell, whereby the CND either comprises, at its N-terminal end, an amino acid sequence (scar sequence) from the C-terminal amino acids of the MTP, or does not comprise a scar sequence.

In a preferred embodiment, the MTP is cleaved in the plant cell with an efficiency of at least 50%, and/or wherein the CND is present in the plant cell at a greater level than the ND, preferably at a ratio of greater than 2:1, more preferably greater than 3:1 or 4:1.

In a preferred embodiment, the CND has NifD function.

In a further or another embodiment of the above aspects the exogenous polynucleotide encodes a ND which is a fusion polypeptide (NifD-linker-NifK fusion polypeptide) comprising, in order, a NifD amino acid sequence, a linker amino acid sequence (linker) and a NifK polypeptide (NK) amino acid sequence, wherein the linker amino acid sequence has a length of 8-50 residues, preferably about 30 residues, which is translationally fused to the ND and NK. In a preferred embodiment, the ND further comprises a mitochondrial targeting peptide (MTP), wherein the MTP is translationally fused at the N-terminal end of the NifD amino acid sequence. In a most preferred embodiment, the ND is capable of being cleaved within the MTP, or immediately after the MTP, to yield a processed NifD polypeptide (CND) when the exogenous polynucleotide is expressed in the plant cell, whereby the CND either comprises, at its N-terminal end, a scar sequence, or does not comprise a scar sequence.

In an embodiment of the above aspects, the ND or the CND has NifD function, or the ND (NifD-linker-NifK polypeptide) has both NifD and NifK functions. In an embodiment, the NifD polypeptide is an AnfD polypeptide and the NifK polypeptide is an AnfK polypeptide.

In an embodiment of the above aspects, the MTP comprises any of the MTPs disclosed herein, for example, the MTP comprises about 51 amino acids in length from a F1-ATPase γ-subunit MTP.

In an embodiment, the CND comprises a scar sequence of 1 to 45 amino acids in length, preferably 1 to 20 amino acids, more preferably 1-10 or 11-20 amino acids, translationally fused at the N-terminal end of the NifD amino acid sequence.

In a further or another embodiment, the ND or the CND, or both, for example the NifD-linker-NifK polypeptide, are in mitochondria of the plant cell, preferably in mitochondrial matrix (MM) of the plant cell.

In a further embodiment, the ND or the CND, or both, for example the NifD-linker-NifK polypeptide, are predominantly soluble in the plant mitochondria. Preferably, at least 60% or at least 75% of the CND that is in the plant mitochondria is soluble. The extent of solubility is preferably determined as described in the Examples.

In a further or another embodiment, the ND, for example the NifD-linker-NifK polypeptide, comprises an amino acid other than tyrosine (Y) at a position corresponding to amino acid 100 of SEQ ID NO:18.

In an embodiment, the ND, for example the NifD-linker-NifK polypeptide, comprises a glutamine (Q) or lysine (K) at the position corresponding to amino acid 100 of SEQ ID NO:18, or a leucine (L) or methionine (M) or phenylalanine (F) at the position corresponding to amino acid 100 of SEQ ID NO:18.

In another embodiment, the ND comprises Q, K, L, or M at the position corresponding to amino acid 100 of SEQ ID NO:18.

In another embodiment, the ND comprises L or M at the position corresponding to amino acid 100 of SEQ ID NO:18.

In another embodiment, the ND comprises Q, K, or L at the position corresponding to amino acid 100 of SEQ ID NO:18.

In another embodiment, the ND comprises Q, K, or M at the position corresponding to amino acid 100 of SEQ ID NO:18.

In another embodiment, the ND comprises Q, K, or F at the position corresponding to amino acid 100 of SEQ ID NO18.

In a further or another embodiment, the ND, for example the NifD-linker-NifK polypeptide, comprises the sequence RRNX (SEQ ID NO:154) at positions corresponding to amino acids 97-100 of SEQ ID NO:18, wherein X is any amino acid other than Y.

In an embodiment, X is Q or K, or L, M or F, or L or M, or Q, K or L, or Q, K or M, or Q, K, or F.

In a further embodiment, the plant cell comprises one or more exogenous polynucleotide(s), preferably 2-8 exogenous polynucleotides, which encode one or more Nif fusion polypeptides (NF) other than ND, each NF comprising a MTP at the N-terminal end of the NF, and (ii) a Nif polypeptide sequence (NP), wherein each MTP is independently the same or different and each NP is independently the same or different.

In an embodiment, each NF is capable of being cleaved within its MTP, or immediately after the MTP, to yield a processed Nif polypeptide (CNF) when the one or more exogenous polynucleotide(s) are expressed in the plant cell, whereby each CNF either comprises, at its N-terminal end, a scar sequence, or does not comprise a scar sequence.

In an embodiment, at least one of the NF polypeptides is a NifK polypeptide or a NifH polypeptide, or both NifK and NifH polypeptides.

In a further or another embodiment, the plant cell comprises a NK amino acid sequence, wherein the C-terminus of the polypeptide is a wild-type NifK C-terminus, i.e., the NK lacks any artificially added C-terminal extension.

In a further or another embodiment of the above aspects the exogenous polynucleotide encodes a NifE-linker-NifN fusion polypeptide (NifE-linker-NifN) comprising, in order, a NifE amino acid sequence (NE), a linker amino acid sequence (linker) and a NifN polypeptide (NN) amino acid sequence, wherein the linker amino acid sequence has a length of 20-70 residues, preferably about 46 residues, which is translationally fused to the NE and NN. In a preferred embodiment, the NifE-linker-NifN polypeptide comprises a mitochondrial targeting peptide (MTP), wherein the MTP is translationally fused at the N-terminal end of the NE amino acid sequence. In a most preferred embodiment, the NifE-linker-NifN polypeptide is capable of being cleaved within the MTP, or immediately after the MTP, to yield a processed NifD polypeptide (CNE) when the exogenous polynucleotide is expressed in the plant cell, whereby the CNE either comprises, at its N-terminal end, a scar sequence, or does not comprise a scar sequence.

In a further or another embodiment, the linker of the NifE-linker-NifN polypeptide is at least about 30 amino acids, or at least about 40 amino acids, or about amino acids to about 60 amino acids, or about 30 amino acids to about 70 amino acids, or about 30 amino acids to about 60 amino acids, or about 30 amino acids to about 50 amino acids, or about 25 amino acids, or about 30 amino acids, or about 35 amino acids, or about 40 amino acids, or about 45 amino acids, or about 46 amino acids, or about 50 amino acids, or about 55 amino acids, in length. Most preferred, the linker is about 30 amino acids in length for a NifD-linker-NifK fusion polypeptide, and about 46 amino acids in length for a NifE-linker-NifN fusion polypeptide. In this context, “about 30” means 27, 28, 29, 30, 31, 32 or 33 amino acids, and “about 46” means 41, 42, 43, 44, 45, 46, 47, 48, 49, 50 or 51 amino acids.

In a further or another embodiment, the linker is of sufficient length to allow the ND and the NK or the NE and NN to associate in a functional configuration in a plant cell or a bacterial cell. In an embodiment, the linker is between 8 and 50 amino acids in length. Preferably, the linker is at least about 20 amino acids, at least about 25 amino acids, or at least about 30 amino acids in length. More preferably, the linker is between 25 and 35 amino acids in length for a NifD-linker-NifK fusion polypeptide.

In a further or another embodiment, the fusion polypeptide is capable of being cleaved within its MTP, or immediately after the MTP, to yield a processed polypeptide (CDK) when the exogenous polynucleotide is expressed in the plant cell, whereby the CDK comprises in order, an optional scar sequence, the NifD amino acid sequence, the linker amino acid sequence and the NK amino acid sequence. If cleavage occurs immediately after the MTP, no scar peptide is present.

In an embodiment, the plant cell comprises the fusion polypeptide, the CDK, or both.

In a further or another embodiment, the CDK comprises a scar sequence of 1 to 45 amino acids in length, preferably 1 to 20 amino acids, more preferably 1-10 or 11-20 amino acids, translationally fused at the N-terminal end of the NifD amino acid sequence.

In a further or another embodiment, the CDK has both NifD and NifK function.

In a further or another embodiment, the plant cell further comprises one or more exogenous polynucleotide(s) which encode one or more Nifpolypeptides (NF) other than ND and NK, each NF comprising (i) a MTP at the N-terminal end of the NF, and (ii) a Nifpolypeptide sequence (NP), wherein each MTP is independently the same or different and each NP is independently the same or different.

In a further of another embodiment, each NF is capable of being cleaved within its MTP, or immediately after the MTP, to yield a processed Nif polypeptide (CNF) when the one or more exogenous polynucleotide(s) are expressed in the plant cell, whereby each CNF either comprises, at its N-terminal end, a scar sequence, or does not comprise a scar sequence.

In an embodiment, at least one of the NF polypeptides is a NifH polypeptide.

In an embodiment, of any of the above aspects, the plant cell comprises exogenous polynucleotides encoding Nif polypeptides comprising (i) NifD, NifH, NifK, NifB, NifE and NifN polypeptides, preferably in the mitochondrial matrix of the plant cell.

In a further or another embodiment of any of the above aspects, each MTP comprises at least 10 amino acids, preferably has a length between 10 and 80 amino acids.

In a further or another embodiment of any of the above aspects, the MTP, or at least one MTP, or all of the MTPs independently comprise an MTP of a mitochondrial protein precursor, or a variant thereof, preferably a plant MTP.

In a further or another embodiment of any of the above aspects, one or more or all of the exogenous polynucleotide(s) are integrated into the nuclear genome of the cell, preferably as a contiguous nucleic acid sequence and/or are expressed in the nucleus of the cell.

In an embodiment of any of the above aspects, the cell is a cell other than an Arabidopsis thaliana protoplast or other than a Nicotiana benthamiana cell.

The present inventors have also produced plant cells which produce combinations of Nif polypeptides which are at least partially soluble in the plant mitochondria.

Thus, in an aspect, the present invention provides a plant cell comprising mitochondria and at least 3, at least 4, at least 5, at least 6, at least 7, at least 8, at least 9, at least 10 or at least 11 Nif polypeptides, wherein the Nif polypeptides are selected from the group consisting of NifF, NifM, NifN, NifS, NifU, NifW, NifY, NifZ, NifV, NifH and NifD-NifK, and wherein each of the at least 3, at least 4, at least 5, at least 6, at least 7, at least 8, at least 9, at least 10 or at least 11 Nif polypeptides are at least partially soluble in the mitochondria.

In an embodiment, the plant cell comprises a NifV polypeptide. Preferably, the NifV produces homocitrate. More preferably, the NifV polypeptide is at least partially soluble in the mitochondria of the plant cell. In an embodiment, the NifV polypeptide is a NifV of the invention.

In another embodiment, the plant cell comprises at least NifS, NifU, or both NifS and NifU polypeptides, and optionally NifV polypeptides.

In another embodiment, the plant cell comprises at least NifH, NifM, or both NifH and NifM polypeptides, and optionally one or more or all of NifV, NifS and NifU.

In another embodiment, the plant cell comprises NifF, NifH or NifD-NifK polypeptides, or NifH and NifD-NifK, or NifF, NifH and NifD-NifK, and optionally one or more or all of NifV, NifS, NifU, NifH and NifM polypeptides.

In an embodiment, the NifD polypeptide is an AnfD polypeptide, the NifH polypeptide is an AnfH polypeptide, and the NifD-NifK polypeptide is an AnfD-AnfK polypeptide. In a preferred embodiment, the plant cell further comprises an AnfG polypeptide which is at least partially soluble in the mitochondria.

In an embodiment, each of the at least 3, at least 4, at least 5, at least 6, at least 7, at least 8, at least 9, at least 10 or at least 11 Nif polypeptides after cleavage by MPP is independently at least 10%, at least 20%, at least 30%, at least 40%, or at least 50% soluble in the mitochondria. The Nif polypeptides may be up to 80% or up to 90% or even fully soluble in mitochondria of the plant cell.

In an embodiment, the at least 3, at least 4, at least 5, at least 6, at least 7, at least 8, at least 9, at least 10 or at least 11 of the Nif polypeptides each independently comprises a mitochondrial targeting peptide (MTP), or a C-terminal peptide resulting from cleavage of a MTP, or a combination of both MPP-processed and unprocessed forms is present, preferably wherein the MTP is at the N-terminus of each of the at least 3, at least 4, at least 5, at least 6, at least 7, at least 8, at least 9, at least 10 or at least 11 Nif polypeptides, or the MPP-processed form does not have a C-terminal peptide at the N-terminus of the Nif polypeptide.

In an embodiment, each MTP is independently cleaved in the plant cell with an efficiency of at least 50%, and/or wherein each of the at least 3, at least 4, at least 5, at least 6, at least 7, at least 8, at least 9, at least 10 or at least 11 processed Nif polypeptides is independently present in the plant cell at a greater level than the corresponding Nif polypeptide, preferably at a ratio of greater than 1:1, greater than 2:1, greater than 3:1 or greater than 4:1.

In an embodiment, the plant cell comprises a NifD-linker-NifK fusion polypeptide comprising, in order, a NifD amino acid sequence (ND), a linker amino acid sequence and a NifK polypeptide (NK) amino acid sequence, wherein the linker amino acid sequence has a length of 8-50 residues, preferably 16-50 residues, more preferably about 26 or about 30 residues, or most preferably is 26 or 30 residues, which is translationally fused to the ND and NK.

In a further embodiment, the NifD-linker-NifK fusion polypeptide comprises a mitochondrial targeting peptide (MTP), or a C-terminal peptide resulting from cleavage of a MTP, or a combination of both MPP-processed and unprocessed forms is present, wherein the MTP is translationally fused at the N-terminal end of the NifD-NifK fusion polypeptide.

In an embodiment, the at least 3, at least 4, at least 5, at least 6, at least 7, at least 8, at least 9, at least 10 or at least 11 processed Nif polypeptides each independently comprises a C-terminal peptide resulting from cleavage of an MTP of 1 to 45 amino acids in length, preferably 1 to 20 amino acids, more preferably 1-10 or 11-20 amino acids, translationally fused at the N-terminal end of the Nif polypeptide.

In an embodiment, the at least 3, at least 4, at least 5, at least 6, at least 7, at least 8, at least 9, at least 10 or at least 11 Nif polypeptides or the at least 3, at least 4, at least 5, at least 6, at least 7, at least 8, at least 9, at least 10 or at least 11 processed Nif polypeptides are functional Nif polypeptides.

In an embodiment, the at least 3, at least 4, at least 5, at least 6, at least 7, at least 8, at least 9, at least 10 or at least 11 Nif polypeptides or preferably the at least 3, at least 4, at least 5, at least 6, at least 7, at least 8, at least 9, at least 10 or at least 11 processed Nif polypeptides, are in mitochondria of the plant cell, preferably in the mitochondrial matrix (MM) of the plant cell.

In an embodiment, the at least 3, at least 4, at least 5, at least 6, at least 7, at least 8, at least 9, at least 10 or at least 11 Nif polypeptides or preferably the at least 3, at least 4, at least 5, at least 6, at least 7, at least 8, at least 9, at least 10 or at least 11 processed Nif polypeptides, or both, are independently predominantly soluble in the plant mitochondria (i.e., greater than 50% soluble in the mitochondria). The processed Nif polypeptides are preferably up to 80% or up to 90% or even fully soluble in mitochondria of the plant cell. Polypeptide solubility may be determined as described herein.

In an embodiment, the NifD fusion polypeptide or the NifD-linker-NifK fusion polypeptide, or MPP-cleaved products thereof, is present in the plant cell and is (a) resistant to protease cleavage at a site within an amino acid sequence corresponding to amino acids 97-100 of SEQ ID NO:18 and/or (b) comprises an amino acid sequence other than RRNY (SEQ ID NO:101) at positions corresponding to amino acids 97-100 of SEQ ID NO:18. In an embodiment, the ND comprises an amino acid other than tyrosine (Y) at a position corresponding to amino acid 100 of SEQ ID NO:18. In an embodiment, the ND comprises a glutamine (Q) or lysine (K) at the position corresponding to amino acid 100 of SEQ ID NO:18, or a leucine (L) or methionine (M) or a phenylalanine (F) at the position corresponding to amino acid 100 of SEQ ID NO:18.

In an embodiment, the MTP is about 51 amino acids in length from a F1-ATPase γ-subunit MTP.

In an embodiment, the plant cell comprises a NK amino acid sequence, wherein the C-terminus of the polypeptide is a wild-type NifK C-terminus.

In an embodiment, the linker is at least about 20 amino acids, or at least about 30 amino acids, or at least about 40 amino acids, or about 20 amino acids to about 70 amino acids, or about 30 amino acids to about 70 amino acids, or about 30 amino acids to about 60 amino acids, or about 30 amino acids to about 50 amino acids, or about 25 amino acids, or about 30 amino acids, or about 35 amino acids, or about 40 amino acids, or about 45 amino acids, or about 46 amino acids, or about 50 amino acids, or about 55 amino acids, in length.

In an embodiment, the NifD-linker-NifK fusion polypeptide is capable of being cleaved within its MTP, or immediately after the MTP, to yield a processed polypeptide (CDK), whereby the CDK comprises in order, an optional C-terminal peptide resulting from cleavage of an MTP, the NifD amino acid sequence (ND), the linker amino acid sequence and the NK amino acid sequence.

In an embodiment, the plant cell further comprises the fusion polypeptide or the CDK, or both.

In an embodiment, the CDK comprises a scar sequence of 1 to 45 amino acids in length, preferably 1 to 20 amino acids, more preferably 1-10 or 11-20 amino acids, translationally fused at the N-terminal end of the NifD amino acid sequence.

In an embodiment, the CDK has both NifD and NifK function.

In an embodiment, the ND is an AnfD and the NK is an AnfK In an embodiment, the MTP is about 51 amino acids in length from a F1-ATPase γ-subunit MTP.

In an embodiment, each MTP comprises at least 10 amino acids, preferably has a length between 10 and 80 amino acids.

In an embodiment, the MTP, or at least one MTP, or all of the MTPs independently comprise an MTP of a mitochondrial protein precursor, or a variant thereof, preferably a plant MTP.

In an embodiment, the at least 3, at least 4, at least 5, at least 6, at least 7, at least 8, at least 9, at least 10 or at least 11 Nif polypeptides are encoded by at least 3, at least 4, at least 5, at least 6, at least 7, at least 8, at least 9, at least 10 or at least 11exogenous polynucleotide(s), at least 3, at least 4, at least 5, at least 6, at least 7, at least 8, at least 9, at least 10 or at least 11 of which are integrated into the nuclear genome of the cell, preferably as a contiguous nucleic acid sequence, and/or are expressed in the nucleus of the plant cell.

In another embodiment of any of the above aspects, the cell is a cell other than an Arabidopsis thaliana protoplast or other than a Nicotiana benthamiana cell.

The present inventors have also successfully expressed, in plant mitochondria, the combination of Nif polypeptides required for a minimal nitrogenase complex.

Thus, in another aspect, the present invention provides a plant cell comprising mitochondria and exogenous polynucleotides which encode at least 8 or at least 9 Nif fusion polypeptides, wherein the exogenous polynucleotides each comprise a promoter which is operably linked to a nucleotide sequence which encodes one of the Nif fusion polypeptides and which expresses the nucleotide sequence in the plant cell, wherein each Nif fusion polypeptide independently comprises a mitochondrial targeting peptide (MTP), wherein the Nif fusion polypeptides comprise (i) NifH, NifB, NifF, NifJ, NifS, NifU and NifV fusion polypeptides and either (ii) a NifD fusion polypeptide and a NifK fusion polypeptide or (iii) a NifD-linker-NifK fusion polypeptide which comprises a NifD sequence having a C-terminus, an oligopeptide linker and a NifK sequence having a N-terminus, wherein the oligopeptide linker is translationally fused to the C-terminus of the NifD sequence and the N-terminus of the NifK sequence, wherein mitochondrial processing protease (MPP)-cleaved products of at least the NifH, NifF, NifS and NifU fusion polypeptides are each at least partially soluble in mitochondria of a plant cell, wherein MPP-cleaved products of the NifD and NifK fusion polypeptides of (ii) if present in the plant cell are at least partially soluble in mitochondria of a plant cell, or a MPP-cleaved product of the NifD-linker-NifK fusion polypeptide of (iii) if present in the plant cell is at least partially soluble in mitochondria of a plant cell, and wherein the NifV fusion polypeptide and/or a MPP-cleaved product thereof produces homocitrate in the plant cell and is at least partially soluble in mitochondria of a plant cell.

In another aspect, the present invention provides a plant cell comprising mitochondria and exogenous polynucleotides which encode at least 2, at least 3, at least 4, at least 5 or at least 6 Nif fusion polypeptides, wherein the exogenous polynucleotides each comprise a promoter which is operably linked to a nucleotide sequence which encodes one of the Nif fusion polypeptides and which expresses the nucleotide sequence in the plant cell, wherein each Nif fusion polypeptide independently comprises a mitochondrial targeting peptide (MTP), wherein the Nif fusion polypeptides comprise (i) one or more than one or all of NifW, NifX, NifY, and NifZ fusion polypeptides, and either (ii) a NifD fusion polypeptide and a NifK fusion polypeptide or (iii) a NifD-linker-NifK fusion polypeptide which comprises a NifD sequence having a C-terminus, an oligopeptide linker and a NifK sequence having a N-terminus, wherein the oligopeptide linker is translationally fused to the C-terminus of the NifD sequence and the N-terminus of the NifK sequence, wherein mitochondrial processing protease (MPP)-cleaved products of at least the NifW, NifX, NifY and NifZ fusion polypeptides if present in the plant cell are each at least partially soluble in mitochondria of a plant cell, wherein either MPP-cleaved products of the NifD and NifK fusion polypeptides of (ii) if present in the plant cell are at least partially soluble in mitochondria of a plant cell, or a MPP-cleaved product of the NifD-linker-NifK fusion polypeptide of (iii) if present in the plant cell is at least partially soluble in mitochondria of a plant cell, and wherein the MPP-cleaved products of the NifD fusion polypeptide and NifK fusion polypeptide of ii) or the MPP-cleaved product of the NifD-linker-NifK fusion polypeptide of iii) is present in the plant cell in greater amount than the amount of the MPP-cleaved products of the NifD fusion polypeptide and NifK fusion polypeptide or the MPP-cleaved product of the NifD-linker-NifK fusion polypeptide present in a corresponding plant cell lacking the exogenous polynucleotides encoding the one or more than one or all of NifW, NifX, NifY and NifZ fusion polypeptides of (i).

In another aspect, the present invention provides a plant cell comprising mitochondria and exogenous polynucleotides which encode at least 5, at least 6, at least 7, at least 8 or at least 9 Nif fusion polypeptides, wherein the exogenous polynucleotides each comprise a promoter which is operably linked to a nucleotide sequence which encodes one of the Nif fusion polypeptides and which expresses the nucleotide sequence in the plant cell, wherein each Nif fusion polypeptide independently comprises a mitochondrial targeting peptide (MTP), wherein the Nif fusion polypeptides comprise (i) NifH, NifS and NifU fusion polypeptides and optionally a NifM polypeptide, (ii) one or more than one or all of NifW, NifX, NifY, and NifZ fusion polypeptides and either (iii) a NifD fusion polypeptide and a NifK fusion polypeptide or (iv) a NifD-linker-NifK fusion polypeptide which comprises a NifD sequence having a C-terminus, an oligopeptide linker and a NifK sequence having a N-terminus, wherein the oligopeptide linker is translationally fused to the C-terminus of the NifD sequence and the N-terminus of the NifK sequence, wherein mitochondrial processing protease (MPP)-cleaved products of the NifS and NifU fusion polypeptides are at least partially soluble in mitochondria of a plant cell, wherein MPP-cleaved products of the NifW, NifX, NifY and NifZ fusion polypeptides, if present in the plant cell, are at least partially soluble in mitochondria of a plant cell, wherein MPP-cleaved products of the NifD and NifK fusion polypeptides of (iii), if present in the plant cell, are at least partially soluble in mitochondria of a plant cell, wherein a MPP-cleaved product of the NifD-linker-NifK fusion polypeptide of (iv), if present in the plant cell, is at least partially soluble in mitochondria of a plant cell, and wherein either the MPP-cleaved products of the NifD fusion polypeptide and NifK fusion polypeptide of iii) or the MPP-cleaved product of the NifD-linker-NifK fusion polypeptide of iv) are present in the plant cell as a complex with P-cluster.

In an embodiment, the plant cell comprises a NifH fusion polypeptide which is an AnfH fusion polypeptide, wherein the NifD fusion polypeptide if present is an AnfD fusion polypeptide, the NifK fusion polypeptide if present is an AnfK fusion polypeptide, the NifD-linker-NifK fusion polypeptide if present is an AnfD-linker-AnfK fusion polypeptide, and the plant cell further comprises an exogenous polynucleotide which encodes an AnfG fusion polypeptide which comprises a MTP, wherein the exogenous polynucleotide which encodes the AnfG fusion polypeptide comprises a promoter which is operably linked to a nucleotide sequence which encodes the AnfG fusion polypeptide and which expresses said nucleotide sequence in the plant cell, and wherein a MPP-cleaved product of the AnfG fusion polypeptide is at least partially soluble in mitochondria of a plant cell.

In an embodiment of the above three aspects, the NifD fusion polypeptide or the NifD-linker-NifK fusion polypeptide is present in the plant cell and is (a) resistant to protease cleavage at a site within an amino acid sequence corresponding to amino acids 97-100 of SEQ ID NO:18 and/or (b) comprises an amino acid sequence other than RRNY (SEQ ID NO:101) at positions corresponding to amino acids 97-100 of SEQ ID NO:18. In another aspect, the present invention provides a plant cell comprising mitochondria and exogenous polynucleotides which encode at least 2, at least 3 or 4 Anf fusion polypeptides, wherein the exogenous polynucleotides each comprise a promoter which is operably linked to a nucleotide sequence which encodes one of the Anf fusion polypeptides and which expresses the nucleotide sequence in the plant cell, wherein each Anf fusion polypeptide independently comprises a mitochondrial targeting peptide (MTP), wherein the Anf fusion polypeptides comprise (i) an AnfG fusion polypeptide or AnfG and AnfH fusion polypeptides, and either (ii) an AnfD fusion polypeptide and an AnfK fusion polypeptide or (iii) an AnfD-linker-AnfK fusion polypeptide which comprises an AnfD sequence having a C-terminus, an oligopeptide linker and an AnfK sequence having a N-terminus, wherein the oligopeptide linker is translationally fused to the C-terminus of the AnfD sequence and the N-terminus of the AnfK sequence, wherein mitochondrial processing protease (MPP)-cleaved products of at least the AnfG and AnfH fusion polypeptides if present in the plant cell are each at least partially soluble in mitochondria of a plant cell, wherein either MPP-cleaved products of the AnfD and AnfK fusion polypeptides of (ii) if present in the plant cell are at least partially soluble in mitochondria of a plant cell, or a MPP-cleaved product of the AnfD-linker-AnfK fusion polypeptide of (iii) if present in the plant cell is at least partially soluble in mitochondria of a plant cell, and wherein the MPP-cleaved products of the AnfD fusion polypeptide and the AnfK fusion polypeptide of ii) or the MPP-cleaved product of the AnfD-linker-AnfK fusion polypeptide of iii) if present in the plant cell form a protein complex in the plant cell with the MPP-cleaved product of the AnfG fusion polypeptide.

In some embodiments, the plant cell further comprises one or more exogenous polynucleotides encoding one or more Nif fusion polypeptides as defined herein.

As a person skilled in the art would appreciate, embodiments of Nif polypeptides provided herein will equally apply specifically to the corresponding Nif polypeptide which is an Anf polypeptide. For example, embodiments of NifD, NifK, and NifH polypeptides described herein for one aspect of the invention equally apply specifically to AnfD, AnfK, and AnfH polypeptides respectively.

The present inventors are the first, to their knowledge, to produce a plant cell comprising a NifV polypeptide which is at least partially soluble in mitochondria. Thus, in another aspect the present invention provides a plant cell comprising a NifV polypeptide (NV), wherein the NV is at least partially soluble, preferably at least 50%, at least 60%, at least 70%, at least 80%, at least 90% or even fully soluble in mitochondria of a plant cell, preferably in the MM of the plant cell.

In an embodiment, the NV is capable of, or is, producing homocitrate in the cell.

In an embodiment, the NV polypeptide comprises amino acids having a sequence as provided as any one of SEQ ID NOs: 163, 206 to 209, 211, or 212, a biologically active fragment thereof, or has an amino acid sequence which is at least 30% identical, at least 40% identical, at least 50% identical, at least 60% identical, at least 70% identical, at least 80% identical, at least 90% identical, at least 95% identical, at least 97% identical, or at least 99% identical, to that provided in any one or more of SEQ ID NOs: 163, 206 to 209, 211, or 212, and is capable of producing homocitrate in a cell.

In an embodiment of this aspect, the present invention provides a plant cell comprising mitochondria and an exogenous polynucleotide which encodes a NifV polypeptide (NV), wherein the exogenous polynucleotide comprises a promoter which is operably linked to a nucleotide sequence which encodes the NV and which expresses said nucleotide sequence in the plant cell, wherein the NV produces homocitrate in the plant cell and is at least partially soluble in mitochondria of a plant cell, wherein the exogenous polynucleotide is preferably integrated into the nuclear genome of the plant cell and/or is expressed in the nucleus of the plant cell, and optionally wherein the NV comprises a mitochondrial targeting peptide (MTP).

In another aspect, the present invention provides a plant cell comprising an exogenous polynucleotide which encodes a NifD polypeptide (ND) which is (a) resistant to protease cleavage at a site within an amino acid sequence corresponding to amino acids 97-100 of SEQ ID NO:18, and/or (b) comprises an amino acid sequence other than RRNY (SEQ ID NO:101) at positions corresponding to amino acids 97-100 of SEQ ID NO:18, wherein the exogenous polynucleotide comprises a promoter which is operably linked to a nucleotide sequence which encodes the ND and which expresses said nucleotide sequence in the plant cell, and wherein the NifD polypeptide preferably comprises a MTP.

In some embodiments, the plant cell further comprises one or more exogenous polynucleotides encoding one or more or all of the Nif fusion polypeptides as defined herein that are present in the cell, and/or wherein a cleaved product of the Nif fusion polypeptide is present in the cell. Preferably, the plant cell comprises an exogenous polynucleotide for each Nif fusion polypeptide and/or cleaved product present in the cell.

In an embodiment, the plant cell comprises an exogenous polynucleotide which encodes a NifK polypeptide (NK), wherein the exogenous polynucleotide which encodes the NK comprises a promoter which is operably linked to a nucleotide sequence which encodes the NK and which expresses said nucleotide sequence in the plant cell, wherein the ND has a C-terminus and the NK has an N-terminus, and wherein either (i) the NK comprises a mitochondrial targeting peptide (MTP), or (ii) the ND and NK are translationally fused as a NifD-linker-NifK fusion polypeptide which comprises an oligopeptide linker, wherein the oligopeptide linker is translationally fused to the C-terminus of the ND and the N-terminus of the NK.

In an embodiment, the plant cell comprises an exogenous polynucleotide which encodes a NifH fusion polypeptide (NH), wherein the exogenous polynucleotide which encodes the NH comprises a promoter which is operably linked to a nucleotide sequence which encodes the NH and which expresses said nucleotide sequence in the plant cell, wherein the NH comprises a mitochondrial targeting peptide (MTP), and preferably wherein the NH and/or a MPP-cleaved product thereof is at least partially soluble in mitochondria of a plant cell.

In an embodiment, a MPP-cleaved product of at least one or more or preferably all of the Nif fusion polypeptides is at least partially soluble in mitochondria of a plant cell, preferably wherein a MPP-cleaved product of each of the NifD, NifK and NifD-linker-NifK fusion polypeptides, if present in the plant cell, and the NifH polypeptide is at least partially soluble in mitochondria of a plant cell.

The present inventors are also the first, to their knowledge, to produce a plant cell comprising a NifH polypeptide which is at least partially soluble in mitochondria. Thus, in another aspect the present invention provides a plant cell comprising a NifH polypeptide (NH), wherein the NH is at least partially soluble in mitochondria.

In an embodiment, the NH is encoded by an exogenous polynucleotide, one which is integrated into the nuclear genome of the cell, preferably as a contiguous nucleic acid sequence with exogenous polynucleotides encoding the NifD, NifK and NifD-linker-NifK fusion polypeptides, if present in the plant cell.

In another aspect, the present invention provides a plant cell comprising an exogenous polynucleotide which encodes a NifH fusion polypeptide (NH), wherein the exogenous polynucleotide comprises a promoter which is operably linked to a nucleotide sequence which encodes the NH and which expresses said nucleotide sequence in the plant cell, wherein the NH comprises a mitochondrial targeting peptide (MTP), wherein a MPP-cleaved product of the NH is at least partially soluble in mitochondria of a plant cell, and optionally wherein the exogenous polynucleotide is integrated into the nuclear genome of the plant cell and/or is expressed in the nucleus of the plant cell.

In some embodiments, the plant cell further comprises one or more exogenous polynucleotides encoding one or more Nif fusion polypeptides as defined herein that are present in the cell, and/or wherein a cleaved product of the Nif fusion polypeptide is present in the cell. Preferably, the plant cell comprises an exogenous polynucleotide for each Nif fusion polypeptide and/or cleaved product present in the cell.

In embodiments of each of the above aspects, the plant cell further comprises an exogenous polynucleotide which encodes a NifM polypeptide (NM), wherein the exogenous polynucleotide which encodes the NM comprises a promoter which is operably linked to a nucleotide sequence which encodes the NM and which expresses said nucleotide sequence in the plant cell, and wherein the NM optionally comprises a mitochondrial targeting peptide (MTP).

In embodiments of each of the above aspects, the plant cell comprises exogenous polynucleotides which encode NifS and NifU fusion polypeptides, wherein the exogenous polynucleotides each comprise a promoter which is operably linked to a nucleotide sequence which encodes one of the Nif fusion polypeptides and which expresses the nucleotide sequence in the plant cell, and wherein the NifS and NifU fusion polypeptides each comprise a mitochondrial targeting peptide (MTP).

In embodiments of each of the above aspects, each Nif polypeptide is produced in the plant cell as a Nif fusion polypeptide comprising a mitochondrial targeting peptide (MTP), wherein each MTP is independently the same or different, preferably wherein the MTP is at the N-terminus of at least one or more than one or all of the Nif fusion polypeptides.

In embodiments of each of the above aspects, each Nif fusion polypeptide produced in the plant cell is independently cleaved by MPP either (i) within the MTP sequence to yield a MPP-cleaved Nif polypeptide, whereby the MPP-cleaved Nif polypeptide comprises, at its N-terminal end, a C-terminal peptide from the MTP (scar peptide), or (ii) immediately after the MTP whereby the MPP-cleaved Nif polypeptide does not comprise a C-terminal peptide from the MTP.

In embodiments of each of the above aspects, each MTP is independently cleaved in the plant cell with an efficiency of at least 50%, and/or wherein each cleaved Nif polypeptide is independently present in the plant cell at a greater level than a corresponding uncleaved Nif fusion polypeptide, preferably at a ratio of greater than 1:1, 2:1 or 3:1.

In embodiments of each of the above aspects, each Nif fusion polypeptide is at least partially cleaved in its MTP sequence in the plant cell to produce a MPP-cleaved Nif polypeptide, wherein each MPP-cleaved Nif polypeptide independently comprises a peptide (scar peptide) of 1 to 45 amino acids in length, preferably 1 to 20 amino acids, more preferably 1 to 11 amino acids or 11 to 20 amino acids derived from the MTP sequence, translationally fused at the N-terminal end of the MPP-cleaved Nif polypeptide. In embodiments, one or more of the scar peptides are independently 2, 3, 4, 5, 6, 7, 8, 9 or 10 amino acids in length. In embodiments, one or more of the scar peptides are independently 11, 12, 13, 14, 15, 16, 17, 18, 19 or 20 amino acids in length, or 20-30, 20-40 or 20-50 mino acids in length even though shorter scar sequences are preferred. In these embodiments, as used herein, the scar peptide includes any linker sequence such as, for example the Gly-Gly linker used in the Examples herein, fused to the N-terminus of the Nif sequence. In embodiments, the Nif sequence retains a Met (translation start Met) from its wild-type sequence at its N-terminus, which Met is not included in the scar sequence. Alternatively, the translation start Met is omitted from the Nif sequence. In embodiments, additional amino acids may be trimmed from the N-terminus of the Nif sequence relative to a corresponding wild-type Nif sequence, provided that the Nif sequence after trimming retains its Nif function.

In embodiments of each of the above aspects, the plant cell further comprises an exogenous polynucleotide which encodes a ferredoxin fusion polypeptide, preferably a FdxN fusion polypeptide, wherein the exogenous polynucleotide which encodes the ferredoxin fusion polypeptide comprises a promoter which is operably linked to a nucleotide sequence which encodes the ferredoxin fusion polypeptide and which expresses said nucleotide sequence in the plant cell, and wherein the ferredoxin fusion polypeptide comprises a mitochondrial targeting peptide (MTP).

In an embodiment, a MPP-cleaved product of the ferredoxin fusion polypeptide is at least partially soluble in mitochondria of a plant cell, and preferably wherein the exogenous polynucleotide is integrated into the nuclear genome of the plant cell and/or is expressed in the nucleus of the plant cell.

In an embodiment, the plant cell comprises a NifD-linker-NifK fusion polypeptide comprising, in order, a NifD amino acid sequence (ND), an oligopeptide linker and a NifK polypeptide (NK) amino acid sequence, wherein the oligopeptide linker has a length of 8-50 residues, preferably 16-50 residues in length, more preferably about 26 or about 30 residues in length, or most preferably is 30 residues in length, which is translationally fused to the ND and NK.

In an embodiment, each Nif fusion polypeptide is cleaved in the plant cell to produce a Nif polypeptide which is a functional Nif polypeptide.

In an embodiment, the plant cell comprises an exogenous polynucleotide which encodes a NifD fusion polypeptide (ND) or a NifD-linker-NifK fusion polypeptide, wherein the ND or the NifD-linker-NifK fusion polypeptide comprises an amino acid sequence other than RRNY (SEQ ID NO:101) at positions corresponding to amino acids 97-100 of SEQ ID NO:18, and wherein the ND or the NifD-linker-NifK fusion polypeptide preferably comprises an amino acid other than tyrosine (Y) at a position corresponding to amino acid 100 of SEQ ID NO:18.

In an embodiment, the ND or the NifD-linker-NifK fusion polypeptide comprises a glutamine (Q) or lysine (K) at the position corresponding to amino acid 100 of SEQ ID NO:18, or a leucine (L) or methionine (M) or a phenylalanine (F) at the position corresponding to amino acid 100 of SEQ ID NO:18.

In an embodiment, the plant cell comprises an exogenous polynucleotide which encodes a NifK fusion polypeptide or a NifD-linker-NifK fusion polypeptide, wherein the NifK fusion polypeptide or the NifD-linker-NifK fusion polypeptide has a C-terminal amino acid sequence which is the same as a C-terminal amino acid sequence of a wild-type NifK polypeptide. In some embodiments, at least the last two, at least the last three, at least the last four amino acids of the sequence are the same as that of a wild-type NifK polypeptide. Suitable wild-type NifK polypeptide sequences include SEQ ID NO:3, as well as Accession numbers WP_049080161.1, WP_044347163.1, SBM87811.1, WP_047370272.1, WP_014333919.1, WP_012728880.1, WP_011912506.1, WP_065303473.1, WP_018989051.1, prf12106319A, WP_011021239.1, and others. In an embodiment, the NifK fusion polypeptide or the NifD-linker-NifK fusion polypeptide, and the MPP-cleaved product therefrom, has an amino acid sequence whereby the last four amino acids of the sequence are the same as the last four amino acids of a wild-type NifK polypeptide.

In an embodiment, the amino acid sequence of the NifK polypeptide of the invention has at its C-terminus the amino acids DLVR (SEQ ID NO:58). In another embodiment, the NifK polypeptide has at its C-terminus the amino acids DLIR (SEQ ID NO:239), DVVR (SEQ ID NO:240), DIIR (SEQ ID NO:241), DLTR (SEQ ID NO:242) or INVW (SEQ ID NO:243). In an embodiment, the AnfK polypeptide has at its C-terminus the amino acids LNVW (SEQ ID NO:244), LNTW (SEQ ID NO:245), LNMW (SEQ ID NO:246), LAMW (SEQ ID NO:247) or LSVW (SEQ ID NO:248).

In embodiments of the above aspects, the plant cell comprises an exogenous polynucleotide which encodes a AnfD-linker-AnfK fusion polypeptide, wherein the AnfD-linker-AnfK fusion polypeptide comprises an AnfD sequence which has a C-terminus, an oligopeptide linker and an AnfK sequence which comprises an N-terminus, wherein the oligopeptide linker is translationally fused to the C-terminus of the AnfD sequence and the N-terminus of the AnfK sequence, wherein the oligopeptide linker has a length of at least about 20 amino acids, at least about 30 amino acids, at least about 40 amino acids, about 20 amino acids to about 70 amino acids, about 30 amino acids to about 70 amino acids, about 30 amino acids to about 60 amino acids, about 30 amino acids to about 50 amino acids, about 25 amino acids, about 30 amino acids, about 35 amino acids, about 40 amino acids, about 45 amino acids, about 46 amino acids, about 50 amino acids or about 55 amino acids. That is, in these embodiments the NifD sequence of the above embodiments is an AnfD sequence and the NifK sequence is an AnfK sequence.

In an embodiment, at least one or more than one or preferably all of the exogenous polynucleotides are integrated into the nuclear genome of the plant cell and/or are expressed in the nucleus of the plant cell.

In an embodiment, each MTP comprises at least 10 amino acids, preferably has a length between 10 and 80 amino acids.

In an embodiment, at least one of the Nif fusion polypeptides comprises an MTP which is about 51 amino acids in length from a F1-ATPase γ-subunit polypeptide.

In an embodiment, the MTP, or at least one MTP, or all of the MTPs independently comprise an MTP of a mitochondrial protein precursor, or a variant thereof, preferably a plant MTP.

In an embodiment, the at least 3, at least 4, at least 5, at least 6, at least 7, at least 8, at least 9, at least 10 or at least 11 Nif polypeptides are encoded by at least 3, at least 4, at least 5, at least 6, at least 7, at least 8, at least 9, at least 10 or at least 11 exogenous polynucleotide(s), at least 3, at least 4, at least 5, at least 6, at least 7, at least 8, at least 9, at least 10 or at least 11 of which are integrated into the nuclear genome of the cell, preferably as a contiguous nucleic acid sequence.

In embodiments of the above aspects, the cell is not capable of giving rise to progeny cells, for example is not capable of regenerating a cell culture or living plant.

In an embodiment, the plant cell of the invention is further defined by one or more of the features mentioned herein. Each possible combination of features is clearly contemplated.

In a further aspect, the present invention provides a plant or plant part, organ or tissue comprising a plant cell of the invention, preferably a transgenic plant or part thereof, wherein the transgenic plant or part thereof is transgenic for at least the one or more exogenous polynucleotide(s) encoding the Nif polypeptide(s).

In an embodiment, the plant part is a seed. In an embodiment, the seed is capable of germinating, or alternatively has been processed or treated so that it is no longer capable of germinating. The cells of the seed may not be capable of regeneration into a cell culture or living plant.

In embodiments of the above aspects, one or more of the one or more exogenous polynucleotide(s) are expressed in roots of a plant, preferably expressed at a greater level in the roots of the plant than in leaves of the plant. In such cases, a promoter sequence is used which provides the desired tissue specificity of expression.

In an embodiment, the transgenic plant has an altered phenotype relative to a corresponding wild-type plant which is increased yield, biomass, growth rate, vigor, nitrogen gain derived from biological nitrogen fixation, nitrogen use efficiency, abiotic stress tolerance, and/or tolerance to nutrient deficiency relative to the corresponding wild-type plant.

In an alternative embodiment, the transgenic plant has the same growth rate and/or phenotype relative to a corresponding wild-type plant.

In embodiments of the above aspects, the plant cell, plant or part thereof is a cereal plant cell, plant or part thereof, such as for example wheat, rice, maize, triticale, oat or barley, preferably wheat.

In embodiments of the above aspects, the plant cell, plant or part thereof is homozygous or heterozygous for the one or more exogenous polynucleotide(s), preferably homozygous for all of the exogenous polynucleotides.

In embodiments of the above aspects, the plant cell, plant or part thereof is a monocotyledonous plant cell, plant or part thereof such as, for example, a cereal plant cell, plant or part thereof such as for example wheat, rice, maize, triticale, oat, or barley, preferably wheat, or a dicotyledonous plant cell, plant or part thereof.

In a further or another embodiment, the transgenic plant is growing in a field or the plant plant part was harvested from a plant that was grown in a field. Alternatively, the plant was grown in a glasshouse.

In a further aspect, the present invention provides a population of at least 100 plants according to the invention growing in a field or in a glasshouse, or plant parts harvested therefrom.

In a another aspect, the present invention provides an isolated or recombinant NifD polypeptide (ND) which is resistant to protease cleavage at a site within an amino acid sequence corresponding to amino acids 97-100 of SEQ ID NO:18.

In a further aspect, the present invention provides an isolated or recombinant NifD polypeptide (ND) which comprises an amino acid sequence other than RRNY (SEQ ID NO:101) at positions corresponding to amino acids 97-100 of SEQ ID NO:18.

The isolated or recombinant ND may be further defined by any of the above recited features which are applicable to Nif polypeptides. All possible combinations of the features recited above are contemplated as part of the invention.

In a related aspect, the present invention provides a NifD fusion polypeptide comprising a mitochondrial targeting peptide (MTP) translationally fused to a NifD polypeptide (ND), or a cleaved product thereof which comprises the ND and optionally a scar peptide, wherein the NifD fusion polypeptide or the cleaved product thereof is (a) resistant to protease cleavage at a site within an amino acid sequence corresponding to amino acids 97-100 of SEQ ID NO:18 and/or (b) comprises an amino acid sequence other than RRNY (SEQ ID NO:101) at positions corresponding to amino acids 97-100 of SEQ ID NO:18.

In an embodiment, the NifD fusion polypeptide comprises an oligopeptide linker and a NifK polypeptide (NK) which are translationally fused as a NifD-linker-NifK fusion polypeptide, wherein the ND comprises a C-terminus and the NK comprises an N-terminus, wherein the oligopeptide linker is translationally fused to the C-terminus of the ND and the N-terminus of the NK.

In another aspect, the present invention provides a cleaved product of the NifD fusion polypeptide of the Mention, wherein the cleaved product comprises the ND, an oligopeptide linker and the NK, wherein the oligopeptide linker is translationally fused to the C-terminus of In an embodiment, the NifD fusion polypeptide or the cleaved product thereof is at least partially soluble in mitochondria of a plant cell when the NifD fusion polypeptide is produced in the plant cell.

In an embodiment, the NifD fusion polypeptide is an AnfD fusion polypeptide, the NK is an AnfK polypeptide, and the NifD-linker-NifK fusion polypeptide is an AnfD-linker-AnfK fusion polypeptide.

In another aspect, the present invention provides a NifK fusion polypeptide comprising a mitochondrial targeting peptide (MTP) translationally fused to a NifK polypeptide (NK), wherein the NifK fusion polypeptide or a cleaved product thereof is at least partially soluble in mitochondria of a plant cell when the NifK fusion polypeptide or the cleaved product thereof is produced in the plant cell.

In another aspect, the present invention provides a cleaved product of the NifK fusion polypeptide of the invention, which comprises the NK and optionally a scar peptide, wherein the cleaved product is at least partially soluble in mitochondria of a plant cell when the cleaved product is produced in the plant cell.

In an embodiment, the NK is an AnfK polypeptide (AK).

In an embodiment, the NifK polypeptide has a C-terminal amino acid sequence which is the same as the C-terminal amino acid sequence of a wild-type NifK polypeptide. Suitable wild-type NifK polypeptide sequences are described herein.

In another aspect, the present invention provides an AnfD fusion polypeptide comprising a mitochondrial targeting peptide (MTP) and an AnfD polypeptide (AD), or a cleaved product thereof comprising the AD and optionally a scar peptide, preferably which is at least partially soluble in mitochondria of a plant cell when the AnfD fusion polypeptide or the cleaved product thereof is produced in the plant cell.

In another aspect, the present invention provides an AnfH fusion polypeptide comprising a mitochondrial targeting peptide (MTP) and an AnfH polypeptide (AH), or a cleaved product thereof comprising the AH and optionally a scar peptide, preferably which is at least partially soluble in mitochondria of a plant cell when the AnfH fusion polypeptide or the cleaved product thereof is produced in the plant cell.

In another aspect, the present invention provides an AnfG fusion polypeptide comprising a mitochondrial targeting peptide (MTP) and an AnfG polypeptide (AG), or a cleaved product thereof comprising the AG and optionally a scar peptide, preferably which is at least partially soluble in mitochondria of a plant cell when the AnfG fusion polypeptide or the cleaved product thereof is produced in the plant cell.

In another aspect, the present invention provides an AnfD-linker-AnfK fusion polypeptide or a cleaved product thereof, comprising an AnfD polypeptide (AD), an oligopeptide linker and an AnfK polypeptide (AK) which are translationally fused, wherein the AD comprises an N-terminus and a C-terminus, and the AK comprises an N-terminus, wherein the oligopeptide linker is translationally fused to the C-terminus of the AD and the N-terminus of the AK, preferably wherein the fusion polypeptide comprises a mitochondrial targeting peptide (MTP) or the cleaved product comprises a scar peptide translationally fused to the N-terminus of the AD.

In another aspect, the present invention provides a combination of Anf polypeptides, being Anf polypeptides according to the aspects described herein, preferably a combination of the cleaved products of the Anf fusion polypeptides. Preferably, at least one or more or all of the cleaved products comprises a scar peptide e.g. fused at the N-terminus of the Anf polypeptide. Preferred combinations are the AnfD and AnfK, the AnfD, AnfK and AnfG, the AnfD-linker-AnfK and AnfG, more preferably the AnfD, AnfK, AnfG and AnfH, or the AnfD-linker-AnfK, AnfG and AnfH polypeptides. In embodiments, the features of the Nif polypeptides described herein apply to the corresponding Anf polypeptides. In a preferred embodiment, the combination of Anf polypeptides, preferably of the cleaved products, is present in a plant cell, a transgenic plant or part thereof, or a product therefrom as described herein.

In another aspect, the present invention provides a protein complex comprising (i) the cleaved product of the NifD fusion polypeptide, preferably the AnfD fusion polypeptide, (ii) the cleaved product of the NifK fusion polypeptide, preferably the AnfK fusion polypeptide, and optionally (iii) an Fe—S cluster, preferably a P-cluster. Preferably, at least one or more or all of the cleaved products comprises a scar peptide e.g. fused at the N-terminus of the Anf polypeptide.

In another aspect, the present invention provides a protein complex comprising (i) the cleaved products of the AnfD fusion polypeptide and the AnfK fusion polypeptide, and optionally the cleaved product of the AnfG fusion polypeptide, or (ii) the cleaved products of the AnfD-linker-AnfK fusion polypeptide and the AnfG fusion polypeptide, and optionally (iii) an Fe—S cluster, preferably a P-cluster. Preferably, at least one or more or all of the cleaved products comprises a scar peptide e.g. fused at the N-terminus of the Anf polypeptide.

In an embodiment, the protein complex of the invention is in a plant cell, preferably in a mitochondrion of the plant cell, or a transgenic plant or part thereof. In an embodiment, the plant cell, transgenic plant or part thereof comprising the Anf polypeptide, combination of Anf polypeptides or protein complex of the invention is used in a method of the invention as des In another aspect, the present invention provides a substantially purified or recombinant NifV polypeptide (NV) which when expressed in a plant cell is at least partially soluble in the plant mitochondria.

In a related aspect, the present invention provides an isolated or recombinant NifV polypeptide, or a NifV fusion polypeptide comprising a mitochondrial targeting peptide (MTP) translationally fused to a NifV polypeptide (NV), or a cleaved product thereof which comprises the NV and optionally a scar peptide, wherein the NifV polypeptide and/or the NifV fusion polypeptide and/or the cleaved product thereof is at least partially soluble in a plant cell when produced in the plant cell, preferably is at least partially soluble in mitochondria of the plant cell.

In an embodiment, the isolated or recombinant NifV polypeptide or the NifV fusion polypeptide or a cleaved product thereof is capable of producing homocitrate in a plant cell, preferably in mitochondria of a plant cell.

In another aspect, the present invention provides a substantially purified or recombinant NifH polypeptide (NH) which when expressed in a plant cell, preferably in a transgenic plant, is at least partially soluble in the plant mitochondria.

In another aspect, the present invention provides a NifH fusion polypeptide comprising a mitochondrial targeting peptide (MTP) translationally fused to a NifH polypeptide (NH), or a cleaved product thereof which comprises the NH and optionally a scar peptide, wherein the NifH fusion polypeptide and/or the cleaved product thereof is at least partially soluble in mitochondria of a plant cell. In embodiments of these aspects, the NH polypeptide is at least partially cleaved in its MTP sequence in the plant cell to produce a MPP-cleaved Nif polypeptide, wherein the MPP-cleaved NH comprises a peptide (scar peptide) of 1 to 45 amino acids in length, preferably 1 to 20 amino acids, more preferably 1 to 11 amino acids or 11 to 20 amino acids derived from the MTP sequence, translationally fused at the N-terminal end of the NH. In embodiments, one or more of the scar peptides are independently 2, 3, 4, 5, 6, 7, 8, 9 or 10 amino acids in length. In embodiments, one or more of the scar peptides are independently 11, 12, 13, 14, 15, 16, 17, 18, 19 or 20 amino acids in length, or 20-30, 20-40 or 20-50 mino acids in length even though shorter scar sequences are preferred.

In an embodiment of these aspects, the NH is an AnfH polypeptide.

In an embodiment, the NifH fusion polypeptide or preferably its MPP-cleavage product is bound to one or two Fe—S clusters, preferably one or two Fe₄-S₄ clusters.

In another aspect, provided is an isolated or exogenous polynucleotide encoding a NifV polypeptide (NV), wherein the NV when expressed in a plant cell is at least partially soluble in the plant mitochondria.

In an embodiment, the NV polypeptide comprises amino acids having a sequence as provided as any one of SEQ ID NO's: 163, 206 to 209, 211, or 212, a biologically active fragment thereof, or has an amino acid sequence which is at least 30% identical, at least 40% identical, at least 50% identical, at least 60% identical, at least 70% identical, at least 80% identical, at least 90% identical, at least 95% identical, at least 97% identical, or at least 99% identical, to that provided in any one or more of SEQ ID NO's: 163, 206 to 209, 211, or 212.

In an embodiment, a polypeptide of the invention is an isolated or recombinant polypeptide. In another embodiment, a polypeptide of the invention such as, for example, a recombinant polypeptide is present in a cell, preferably in a plant cell.

Suitable amino acid sequences for the Nif polypeptides of any of the above aspects are known in the art and include those provided herein.

In an embodiment, the NifH polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:1;
ii. SEQ ID NO:218;
iii. SEQ ID NO:224;
iv. Accession No. WP_049123239.1;
v. Accession No. WP_048638817.1;
vi. Accession No. WP_013029017.1;
vii. Accession No. WP_013010353.1;
viii. Accession No. WP_014258951.1;
ix. Accession No. WP_011744626.1;
x. Accession No. WP_013718497.1;
xi. Accession No. WP_009565928.1;
xii. Accession No. WP_013099472.1;
xiii. Accession No. WP_007781874.1;
xiv. Accession No. WP_012703362;
xv. Accession No. WP_153472986;
xvi. Accession No. WP_015854293;
xvii. Accession No. WP_123927773;
xviii. Accession No. WP_073538802; and
xix. Accession No. RCV6483.

In an embodiment, the NifH polypeptide comprises one or more of the amino acid sequence motifs provided in SEQ ID NOs:225-231.

In an embodiment, the NifH polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:1.

In an embodiment, the NifH polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:218.

In an embodiment, the NifD polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:2;
ii. SEQ ID NO:18;
iii. SEQ ID NO:148;
iv. SEQ ID NO:149;
v. SEQ ID NO:150;
vi. SEQ ID NO:151;
vii. SEQ ID NO:152;
viii. SEQ ID NO:153;
ix. SEQ ID NO:216;
x. Accession No. WP_044347161.1;
xi. Accession No. WP_047370273.1;
xii. Accession No. WP_038902190.1;
xiii. Accession No. WP_024872642.1;
xiv. Accession No. WP_024078601.1;
xv. Accession No. WP_013298320.1;
xvi. Accession No. WP_010877172.1;
xvii. Accession No. WP_014258953.1;
xviii. Accession No. WP_066665786.1;
xix. Accession No. WP_015773055.1;
xx. Accession No. WP_016867598.1;
xxi. Accession No. WP_009512873.1;
xxii. Accession No. WP_012703361;
xxiii. Accession No. WP_075356167;
xxiv. Accession No. WP_038590013;
xxv. Accession No. WP_023922817;
xxvi. Accession No. WP_011021232; and
xxvii. Accession No. OAV73823.

In an embodiment, the NifD polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:2.

In an embodiment, the NifD polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:216.

In an embodiment, the NifK polypeptide comprises amino acids having a sequence which is at least 30% identical, at least 40% identical, at least 50% identical, at at least 95% identical, at least 97% identical, or at least 99% identical, to any one or more of the following sequences:
i. SEQ ID NO:3;
ii. SEQ ID NO:217;
iii. Accession No. WP_049080161.1;
iv. Accession No. WP_044347163.1;
v. Accession No. SBM87811.1;
vi. Accession No. WP_047370272.1;
vii. Accession No. WP_014333919.1;
viii. Accession No. WP_012728880.1;
ix. Accession No. WP_011912506.1;
x. Accession No. WP_065303473.1;
xi. Accession No. WP_018989051.1;
xii. Accession No. prf12106319A;
xiii. Accession No. WP_011021239.1;
xiv. Accession No. WP_012703359;
xv. Accession No. WP_144571040;
xvi. Accession No. WP_077859050;
xvii. Accession No. WP_122630336; and
xviii. Accession No. WP_088520366.

In an embodiment, the NifK polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:3.

In an embodiment, the NifK polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:217.

In an embodiment, the NifB polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:4;
ii. Accession No. WP_041145602.1;
iii. Accession No. WP_043953592.1;
iv. Accession No. WP_040003311.1;
v. Accession No. WP_011094468.1;
vi. Accession No. WP_048638849.1;
vii. Accession No. WP_011813098.1;
viii. Accession No. WP_048108879.1;
ix. Accession No. WP_050355163.1;
x. Accession No. WP_015850328.1; and
xi. Accession No. P10930.

In an embodiment, the NifB polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:4.

In an embodiment, the NifE polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:5;
ii. Accession No. WP_049114606.1;
iii. Accession No. SBM87755.1;
iv. Accession No. WP_012764127.1;
v. Accession No. WP_012728883.1;
vi. Accession No. WP_003297989.1;
vii. Accession No. WP_012698965.1;
viii. Accession No. WP_013190624.1;
ix. Accession No. WP_025698318.1;
x. Accession No. WP_013460149.1;
xi. Accession No. AIS31022.1;
xii. Accession No. WP_018701501.1; and
xiii. Accession No. WP_048514099.1.

In an embodiment, the NifE polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:5.

In an embodiment, the NifF polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:6;
ii. Accession No. WP_004122417.1;
iii. Accession No. WP_040968713.1;
iv. Accession No. WP_035885760.1;
v. Accession No. WP_039999438.1;
vi. Accession No. WP_048638838.1;
vii. Accession No. WP_064006977.1;
viii. Accession No. WP_012698862.1;
ix. Accession No. WP_010933399.1;
x. Accession No. WP_002949173.1; and
xi. Accession No. WP_039801725.1.

In an embodiment, the NifF polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:6.

In an embodiment, the AnfG polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:219;
ii. Accession No. WP_012703360;
iii. Accession No. WP_144571041;
iv. Accession No. HBE76208;
v. Accession No. WP_144349445;
vi. Accession No. WP_112317428; and
vii. Accession No. WP_048515315.

In an embodiment, the AnfG polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:219.

In an embodiment, the NifJ polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:7;
ii. Accession No. WP_024360006.1;
iii. Accession No. WP_044347157.1;
iv. Accession No. WP_050533844.1;
v. Accession No. WP_064566543.1;
vi. Accession No. WP_057084649.1;
vii. Accession No. WP_014683040.1;
viii. Accession No. WP_013149847.1;
ix. Accession No. WP_053341220.1;
x. Accession No. WP_014454638.1; and
xi. Accession No. CSA83023.1.

In an embodiment, the NifJ polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:7.

In an embodiment, the NifM polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:8;
ii. Accession No. WP_064342940.1;
iii. Accession No. WP_004122413.1;
iv. Accession No. WP_044347181.1;
v. Accession No. WP_064566543.1;
vi. Accession No. WP_063105800.1;
vii. Accession No. WP_035885759.1;
viii. Accession No. WP_011094472.1;
ix. Accession No. WP_048638837.1;
x. Accession No. CAA75544.1;
xi. Accession No. WP_051692859.1; and
xii. Accession No. WP_018415157.1.

In an embodiment, the NifM polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:8.

In an embodiment, the NifN polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:9;
ii. Accession No. WP_064391778.1;
iii. Accession No. WP_047370268.1;
iv. Accession No. WP_014683026.1;
v. Accession No. WP_048638830.1;
vi. Accession No. WP_027147663.1;
vii. Accession No. WP_015195966.1;
viii. Accession No. WP_023593609.1;
ix. Accession No. WP_025677480.1; and
x. Accession No. WP_018306265.1.

In an embodiment, the NifN polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:9.

In an embodiment, the NifQ polypeptide comprises amino acids having a sequence which is at least 30% identical, at least 40% identical, at least 50% identical, at at least 95% identical, at least 97% identical, or at least 99% identical, to any one or more of the following sequences:
i. SEQ ID NO:10;
ii. Accession No. WP_064391765.1;
iii. Accession No. CTQ06350.1;
iv. Accession No. WP_047370257.1;
v. Accession No. WP_043878077.1;
vi. Accession No. WP_008878174.1;
vii. Accession No. WP_011501504.1;
viii. Accession No. WP_027196569.1;
ix. Accession No. GAU06296.1; and
x. Accession No. WP_063239464.1.

In an embodiment, the NifQ polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:10.

In an embodiment, the NifS polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:11;
ii. SEQ ID NO:19;
iii. Accession No. WP_004138780.1;
iv. Accession No. WP_045858151.1;
v. Accession No. WP_047370265.1;
vi. Accession No. WP_014333911.1;
vii. Accession No. WP_055731597.1;
viii. Accession No. WP_014239770.1;
ix. Accession No. WP_054691765.1;
x. Accession No. WP_021802294.1;
xi. Accession No. WP_026894054.1; and
xii. Accession No. WP_061575621.1.

In an embodiment, the NifS polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:11.

In an embodiment, the NifS polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:19.

In an embodiment, the NifU polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:12;
ii. Accession No. WP_049136164.1;
iii. WP_050887862.1;
iv. WP_057084657.1;
v. WP_048638833.1;
vi. WP_012728889.1;
vii. WP_055731596.1;
viii. WP_028587630.1;
ix. WP_044417303.1;
x. WP_001051984.1; and
xi. KIM05011.1.

In an embodiment, the NifU polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:12.

In an embodiment, the NifV polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:13;
ii. SEQ ID NO:163;
iii. SEQ ID NO:164;
iv. SEQ ID NO:206;
v. SEQ ID NO:207;
vi. SEQ ID NO:208;
vii. SEQ ID NO:209;
viii. SEQ ID NO:210;
ix. SEQ ID NO:211;
x. SEQ ID NO:212;
xi. SEQ ID NO:213;
xii. SEQ ID NO:214;
xiii. SEQ ID NO:215;
xiv. Accession No. WP_049083341.1;
xv. Accession No. WP_045858154.1;
xvi. Accession No. WP_047370264.1;
xvii. Accession No. WP_038912041.1;
xviii. Accession No. WP_048638835.1;
xix. Accession No. WP_011712856.1;
xx. Accession No. WP_037528703.1;
xxi. Accession No. OAA29062.1; and
xxii. Accession No. EKQ56006.1.

In an embodiment, the NitV polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:13.

In an embodiment, the NifX polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:14;
ii. Accession No. WP_049070199.1;
iii. Accession No. WP_064342937.1;
iv. Accession No. WP_044347173.1;
v. Accession No. WP_044612922.1;
vi. Accession No. WP_043953583.1;
vii. Accession No. WP_039999416.1;
viii. Accession No. WP_047608097.1;
ix. Accession No. WP_039800848.1;
x. Accession No. WP_062149047.1; and
xi. Accession No. WP_020165972.1.

In an embodiment, the NifX polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:14.

In an embodiment, the NifY polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:15;
ii. Accession No. WP_049089500.1;
iii. Accession No. WP_064342935.1;
iv. Accession No. WP_044524054.1;
v. Accession No. WP_049010739.1;
vi. Accession No. WP_047370270.1;
vii. Accession No. WP_039999411.1;
viii. Accession No. WP_037382461.1;
ix. Accession No. WP_014683024.1;
x. Accession No. AEX25784.1; and
xi. Accession No. WP_012698835.1.

In an embodiment, the NifY polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:15.

In an embodiment, the NifZ polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:16;
ii. Accession No. WP_057173223.1;
iii. Accession No. WP_064342939.1;
iv. Accession No. WP_043875005.1;
v. Accession No. WP_043953588.1;
vi. Accession No. WP_065368553.1;
vii. Accession No. WP_062627625.1;
viii. Accession No. WP_011491838.1;
ix. Accession No. WP_014029050.1; and
x. Accession No. WP_015665422.1.

In an embodiment, the NifZ polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:16.

In an embodiment, the NifW polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:17;
ii. Accession No. WP_064342938.1;
iii. Accession No. WP_049080155.1;
iv. Accession No. WP_095103586.1;
v. Accession No. WP_065877373.1;
vi. Accession No. WP_095699971.1;
vii. Accession No. WP_012764136.1;
viii. Accession No. WP_053085547.1;
ix. Accession No. WP_077299824.1;
x. Accession No. OGI40729;
xi. Accession No. AC076430.1; and
xii. Accession No. BBA37427.1.

In an embodiment, the NifW polypeptide comprises amino acids having a sequence which is at least 30% identical, at least 40% identical, at least 50% identical, at at least 95% identical, at least 97% identical, at least 99% identical, or is identical to the sequence provided in SEQ ID NO:17.

In an embodiment, the ferredoxin polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:232;
ii. Accession No. WP_012703542;
iii. Accession No. WP_065835964.1;
iv. Accession No. WP_069124666.1;
v. Accession No. WP_101942980;
vi. Accession No. WP_049076934.1;
vii. Accession No. WP_072048756.1;
viii. Accession No. WP_130674512.1; and
ix. Accession No. WP_103805005.1.

In an embodiment, the ferredoxin polypeptide comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:232.

Suitable amino acid sequences for MTPs in relation to any of the above aspects are known in the art and include those provided herein. In an embodiment, the MTP comprises amino acids having a sequence which is at least 30% identical, at least 40% identical, at least 50% identical, at least 60% identical, at least 70% identical, at least 80% identical, at least 90% identical, at least 95% identical, at least 97% identical, or at least 99% identical, to any one or more of the following sequences:
i. SEQ ID NO:36;
ii. SEQ ID NO:21;
iii. amino acids 1-77 of SEQ ID NO:20;
iv. SEQ ID NO:28;
v. SEQ ID NO:29;
vi. SEQ ID NO:30;
vii. SEQ ID NO:31;
viii. SEQ ID NO:32;
ix. SEQ ID NO:33;
x. SEQ ID NO:34;
xi. SEQ ID NO:35;
xii. SEQ ID NO:37; and
xiii. SEQ ID NO:38.

In an embodiment, the MTP comprises amino acids having a sequence which is at least 30% identical, at least 40% identical, at least 50% identical, at least 60% identical, at least 70% identical, at least 80% identical, at least 90% identical, at least 95% identical, at least 97% identical, at least 99% identical, or is identical to the sequence provided in SEQ ID NO:36.

In another aspect, the present invention provides a polynucleotide encoding any one or more of the polypeptides of the invention.

In an embodiment, a protein coding region of the polynucleotide has been codon-modified for expression in a plant cell, relative to a corresponding protein coding region of a naturally-occurring polynucleotide in a bacterium. In an embodiment, most, or even all, of the protein coding regions have been codon-optimised for expression in a plant cell, preferably the plant cell of the invention.

In a further embodiment, each exogenous polynucleotide comprises a promoter operably linked to the polynucleotide and/or translational regulatory elements operably linked to the polynucleotide.

In another embodiment, the promoter confers expression of the one or more polynucleotide(s) in roots, leaves and/or stem of a plant, preferably the promoter confers expression of the one or more polynucleotide(s) in one, or more, or all of the roots, leaves or a stem of the plant relative to seed of the plant.

In another embodiment, the one or more or all of the polynucleotides are present in a plant cell or a bacterial cell, preferably integrated into the nuclear genome of the plant cell, for example as a contiguous DNA sequence integrated into the chloroplast genome or preferably the nuclear genome of the plant cell. The plant cell may contain multiple copies of the contiguous DNA sequence integrated into the nuclear genome, for example as multiple T-DNAs.

In an embodiment, each polynucleotide, or each sequence within it encoding a polypeptide, is operably linked to a promoter and optionally, a transcription termination sequence.

In a further or another embodiment, the promoter confers expression of one, or more of the polynucleotide(s) in roots, leaves and/or stem of a plant, preferably the one or more polynucleotide(s) is preferentially expressed in one, or more, or all of the roots, leaves or a stem of the plant relative to seed of the plant.

In a further aspect provided is a chimeric vector comprising or encoding a polynucleotide of the invention.

In another aspect, the present invention provides a vector comprising the polynucleotide of the invention.

In an embodiment, the vector comprises polynucleotides which encode at least 3, at least 4, or at least 5 Nif fusion polypeptides.

In another aspect, the present invention provides a vector comprising polynucleotides which encode at least 3, at least 4, or at least 5 of the Nif fusion polypeptides defined in any one of the above aspects of the invention.

In an embodiment, the vector comprises polynucleotides encoding
a) the NifD fusion polypeptide and the NifK fusion polypeptide, or the NifD-linker-NifK fusion polypeptide; and
b) the NifH fusion polypeptide and the NifV fusion polypeptide; and
c) optionally, the AnfG fusion polypeptide and/or the ferredoxin fusion polypeptide.

In an embodiment, the vector comprises polynucleotides encoding
a) the NifF, NifJ, NifU and NifB fusion polypeptides and optionally the NifS fusion polypeptide; and/or
b) the Nif W, NifX, NifY and NifZ fusion polypeptides.

In a further aspect, the present invention provides a cell comprising one, or more, of the polypeptides according to the invention, one, or more of the exogenous polynucleotides according to the invention, and/or a vector according to the invention.

In a related aspect, the present invention provides a cell, preferably a plant cell, comprising a fusion polypeptide or cleaved product according to the invention, or a combination of two or more of said fusion polypeptides or cleaved products, a protein complex according to the invention, and/or a polynucleotide according to the invention or a vector according to the invention. In a preferred embodiment, the cell comprises an exogenous polynucleotide for each fusion polypeptide or cleaved product present in the cell.

In an embodiment, the fusion polypeptide or cleaved product, or combination of fusion polypeptides or cleavage products, or protein complex, is in mitochondria of the cell. As would be understood readily, not all of the fusion polypeptide or cleaved product needs to be in the mitochondria of the cell, so long as at least some is in the mitochondria.

In an embodiment, the cell is a plant cell or a bacterial cell, preferably a cell of a transgenic plant, more preferably wherein at least one of exogenous polynucleotides is integrated in the nuclear genome of the cell.

In a further embodiment, the plant cell is a monocotyledonous plant cell such as, for example, a cereal plant cell such as a wheat cell, a rice cell, a maize cell, a triticale cell, an oat cell, or a barley cell, preferably a wheat cell, or a dicotyledonous plant cell. The plant cell may be further characterized by the polypeptides or polynucleotides defined by any of the above recited features. All possible combinations of the features recited above are contemplated as part of the invention in the context of the plant cell, and other aspects of the invention.

In a further aspect, the present invention provides a transgenic plant or a transgenic part thereof, preferably seed, comprising one, or more, of the polypeptides according to the invention, one, or more of the exogenous polynucleotides according to the invention, and/or a vector according to the invention.

In an embodiment, the transgenic plant is a monocotyledonous plant such as, for example, a cereal plant such as wheat, rice, maize, triticale, oat, or barley, preferably wheat, or a dicotyledonous plant. The plant or part thereof may be further characterized by the polypeptides or polynucleotides defined by any of the above recited features. All possible combinations of the features recited above are contemplated as part of the invention in the context of the plant or part thereof, and other aspects of the invention.

In a further aspect, the present invention provides a method of producing a polypeptide according to the invention, the method comprising expressing in a cell a polynucleotide according to the invention.

In a further aspect, the present invention provides a method of producing a cell according to the invention, the method comprising the step of introducing one or more polynucleotides according to the invention, and/or a vector according to the invention, into a cell.

In another aspect, the present invention provides a method of producing homocitrate in a plant cell, the method comprising expressing the recombinant NifV polypeptide or the NifV fusion polypeptide of the invention in the plant cell, wherein the recombinant NifV polypeptide or the NifV fusion polypeptide, and/or a cleaved product thereof, produces homocitrate in the plant cell.

In an embodiment, the method further comprises introducing a polynucleotide encoding the recombinant NifV polypeptide or the NifV fusion polypeptide into the plant cell.

In another aspect, the present invention provides use of the NifV polypeptide of the invention for producing homocitrate in a plant cell.

In another aspect, the present invention provides a method of increasing the amount of a NifD, NifK or NifD-linker-NifK fusion polypeptide in a plant cell, the method comprising expressing one or more or all of NifW, NifX, NifY and NifZ fusion polypeptides in the plant cell, wherein each Nif fusion polypeptide independently comprises a mitochondrial targeting peptide (MTP), wherein the amount of the NifD, NifK or NifD-linker-NifK fusion polypeptide in the plant cell is increased relative to a corresponding plant cell not expressing one or more or all of the NifW, NifX, NifY and

NifZ fusion polypeptides.

In an embodiment, the method further comprises
i) introducing one or more polynucleotides encoding the NifD, NifK or NifD-linker-NifK fusion polypeptide into the plant cell; and
ii) introducing one or more polynucleotides encoding one or more or all of the NifW, NifX, NifY and NifZ fusion polypeptides into the plant cell.

In another aspect, the present invention provides a method of increasing the amount of a NifY polypeptide in a plant cell, the method comprising expressing one or more or all of NifW, NifX and NifZ fusion polypeptides in the plant cell, wherein each Nif fusion polypeptide independently comprises a mitochondrial targeting peptide (MTP), wherein the amount of the NifY polypeptide in the plant cell is increased relative to a corresponding plant cell not expressing one or more or all of the NifW, NifX and NifZ fusion polypeptides.

In an embodiment, the method further comprises
i) introducing a polynucleotide encoding a NifY fusion polypeptide into the plant cell; and
ii) introducing one or more polynucleotides encoding the one or more or all of the NifW, NifX and NifZ fusion polypeptides into the plant cell.

In another aspect, the present invention provides use of one or more polynucleotides encoding one or more or all of NifW, NifX and NifZ fusion polypeptides to increase the amount of a NifY polypeptide in a plant cell.

In another aspect, the present invention provides use of a polynucleotide of the invention, and/or a vector of the invention, for producing a transgenic plant cell.

In another aspect, the present invention provides a method of producing a transgenic plant, the method comprising the steps of
i) introducing one or more polynucleotides of the invention, and/or one or more vectors of the invention, into a cell of a plant,
ii) from the cell of step i), regenerating a transgenic plant of the invention, and
iii) optionally, producing transgenic seed and/or progeny plants from the transgenic plant regenerated in step ii).

In a further aspect, the present invention provides a method of producing transgenic seed, comprising
i) harvesting seed from the transgenic plant of the invention, and/or
ii) harvesting seed from one or more transgenic progeny plants produced by the method of the invention.

In a further aspect, the present invention provides a method of producing a plant which has integrated into its genome a polynucleotide according to the invention, the method comprising the steps of
i) crossing two parental plants, wherein at least one plant comprises the polynucleotide,
ii) screening one or more progeny plants from the cross for the presence or absence of the polynucleotide, and
iii) selecting a progeny plant which comprises the polynucleotide, thereby producing the plant.

In a further or another embodiment, at least one of the parental plants is a tetraploid or hexaploid wheat plant.

In a further or another embodiment, step ii) comprises analysing a sample comprising DNA from the one or more progeny plants for the polynucleotide.

In a further or another embodiment, step iii) comprises
i) selecting a progeny plant which is homozygous for the polynucleotide, and/or
ii) analysing the plant or the one or more progeny plants thereof for presence and/or expression of the polynucleotide or for an altered phenotype as defined above.

In one or a further embodiment, the method further comprises:
iv) backcrossing the progeny of the cross of step i) with a plant of the same genotype as a first parent plant lacking the polynucleotide for a sufficient number of times to produce a plant with a majority of the genotype of the first parent but comprising the polynucleotide, and
v) selecting a progeny plant which comprises the polynucleotide and/or has an altered phenotype as defined above.

In a further or another embodiment, the method further comprises the step of analysing the plant or progeny plant for at least one other genetic marker.

In a further aspect, the present invention provides a plant produced using a method according to the invention.

In a further aspect, the present invention provides use of a polynucleotide according to the invention, and/or a vector according to the invention, to produce a recombinant cell and/or a transgenic plant.

In an embodiment, the transgenic plant has an altered phenotype as defined above when compared to a corresponding plant lacking the exogenous polynucleotide, and/or the vector.

In a further aspect, the present invention provides a method for identifying a plant comprising a polynucleotide according to the invention, the method comprising the steps of
i) obtaining a nucleic acid sample from a plant, and
ii) screening the sample for the presence or absence of the polynucleotide.

In an embodiment, the presence of the polynucleotide indicates that the plant has an altered phenotype as defined above, when compared to a corresponding plant lacking the exogenous polynucleotide.

In a further or another embodiment, the method identifies a plant according to the invention.

In a further or another embodiment, the method further comprises producing a plant from a seed before step i).

In another aspect, the present invention provides a transgenic plant part comprising a plant cell of the invention or obtained from the transgenic plant of the invention.

In an embodiment, the plant part is a seed that comprises the polynucleotide of the invention.

In another aspect, the present invention provides a method of producing flour, wholemeal, starch, oil, seed meal or other product obtained from seed, the method comprising;
a) obtaining the seed of the invention, and/or
b) extracting the flour, wholemeal, starch, oil or other product, or producing the seed meal.

In a further aspect, the present invention provides a product produced from the transgenic plant of the invention and/or the plant part of the invention comprising the polypeptide of the invention and/or the polynucleotide of the invention.

In an embodiment, the plant part is a seed.

In a further or another embodiment, the product is a food ingredient or beverage ingredient or a food product or beverage product. Preferably, i) the food ingredient or product is selected from the group consisting of: flour, starch, oil, leavened or unleavened breads, pasta, noodles, animal fodder, breakfast cereals, snack foods, cakes, malt, pastries and foods containing flour-based sauces, or ii) the beverage product is juice, beer or malt.

Methods of producing such products are well known to those skilled in the art.

In an alternative embodiment, the product is a non-food product. Examples of non-food products include, but are not limited to, films, coatings, adhesives, building materials and packaging materials. Methods of producing such products are well known to those skilled in the art.

In a further aspect, the present invention provides a method of preparing a food product, the method comprising mixing seed of the invention, or flour, wholemeal, starch, oil or other product from the seed, with another food ingredient, or processing the seed or flour or wholemeal, preferably by milling, cracking, polishing, flaking, parboiling, cooking or baking the seed or a composition comprising the seed and/or flour or wholemeal obtained from the seed.

In a further aspect, the present invention provides method of preparing malt, comprising the step of germinating seed according to the invention.

In a further aspect, the present invention provides use of a plant or part thereof according to the invention as animal feed, or to produce feed for animal consumption or food for human consumption.

In a further aspect, the present invention provides a composition comprising a polypeptide according to the invention, a polynucleotide according to the invention, a vector according to the invention, or a cell according to the invention, and one or more acceptable carriers.

In a further aspect, the present invention provides a method for reconstitution of a nitrogenase protein complex in a plant cell, the method comprising introducing two or more polynucleotides according to the invention, two or more nucleic acid constructs according to the invention, and/or a vector according to the invention into the cell, and culturing the plant cell for a sufficient time for the polynucleotides or vector to be expressed.

In another aspect, the present invention provides a plant cell comprising mitochondria and 3, 4, 5, 6, 7, 8, 9, 10 or 11 Nif polypeptides, wherein the Nif polypeptides are selected from the group consisting of NifF, NifM, NifN, NifS, NifU, Nitta, NifY, NifZ, NifV, NifH and NifD-NifK, and wherein each of the 3, 4, 5, 6, 7, 8, 9, 10 or 11 Nif polypeptides are at least partially soluble in the mitochondria.

In an embodiment, the plant cell comprises NifV. Preferably, the NifV produces homocitrate. In an embodiment, the NifV is a NifV of the invention.

In another embodiment, the plant cell comprises NifS, NifU, or both NifS and NifU, and optionally NifV

In another embodiment, the plant cell comprises NifH, NifM, or both NifH and NifM, and optionally one or more of all of NifV, NifS and NifU.

In another embodiment, the plant cell comprises NifF, NifH or NifD-NifK, or NifH and NifD-NifK, or NifF, NifH and NifD-NifK, and optionally one or more of all of NifV, NifS, NifU, NifH and NifM

In an embodiment, the NifH polypeptide is an AnfH polypeptide, and the NifD-NifK polypeptide is an AnfD-AnfK polypeptide.

In a further embodiment, the plant comprises an AnfG polypeptide which is at least partially soluble in the mitochondria.

In an embodiment, each of the 3, 4, 5, 6, 7, 8, 9, 10 or 11 Nif polypeptides is at least 10%, at least 20%, at least 30%, or at least 40%, up to 50% soluble in the mitochondria.

In an embodiment, the 3, 4, 5, 6, 7, 8, 9, 10 or 11 of the Nif polypeptides each independently comprises a mitochondrial targeting peptide (MTP), or a C-terminal peptide resulting from cleavage of a MTP, or both, preferably wherein the MTP or C-terminal peptide or both is at the N-terminus of each of the 3, 4, 5, 6, 7, 8, 9, 10 or 11 Nif polypeptides, or does not have an MTP and does not have a C-terminal peptide at the N-terminus of the Nif polypeptide.

In a further embodiment, the 3, 4, 5, 6, 7, 8, 9, 10 or 11 Nif polypeptides are each independently cleaved within the MTP, or immediately after the MTP, to yield 3, 4, 5, 6, 7, 8, 9, 10 or 11 processed Nif polypeptides, whereby each of the 3, 4, 5, 6, 7, 8, 9, 10 or 11 processed Nif polypeptides comprises, at its N-terminal end, the C-terminal peptide from the MTP, or does not comprise a C-terminal peptide from the MTP.

In another or further embodiment, the 3, 4, 5, 6, 7, 8, 9, 10 or 11 Nif polypeptides are each independently cleaved within the MTP, or immediately after the MTP, to yield 3, 4, 5, 6, 7, 8, 9, 10 or 11 processed Nif polypeptides, whereby each of the 3, 4, 5, 6, 7, 8, 9, 10 or 11 processed Nif polypeptides comprises, at its N-terminal end, the C-terminal peptide from the MTP, or does not comprise a C-terminal peptide from the MTP.

In an embodiment, each MTP is independently cleaved in the plant cell with an efficiency of at least 50%, and/or wherein each of the 3, 4, 5, 6, 7, 8, 9, 10 or 11 processed Nif polypeptides is independently present in the plant cell at a greater level than the corresponding Nif polypeptide, preferably at a ratio of greater than 1:1, 2:1 or 3:1.

In an embodiment, the plant cell comprises NifD-NifK fusion polypeptide comprising, in order, a NifD amino acid sequence (ND), a linker amino acid sequence and a NifK polypeptide (NK) amino acid sequence, wherein the linker amino acid sequence has a length of 8-50 residues, preferably 16-50 residues, more preferably about 26 or about 30 residues, or most preferably is 26 or 30 residues, which is translationally fused to the ND and NK.

In a further embodiment, the NifD-NifK fusion polypeptide comprises a mitochondrial targeting peptide (MTP), or a C-terminal peptide resulting from cleavage of a MTP, or both, wherein the MTP or C-terminal peptide resulting from cleavage of a MTP, or both, is translationally fused at the N-terminal end of the NifD-NifK fusion polypeptide.

In an embodiment, the 3, 4, 5, 6, 7, 8, 9, 10 or 11 processed Nif polypeptides each independently comprises a C-terminal peptide resulting from cleavage of an MTP of 1 to 45 amino acids in length, preferably 1 to 20 amino acids, translationally fused at the N-terminal end of the Nif polypeptide.

In an embodiment, the 3, 4, 5, 6, 7, 8, 9, 10 or 11 Nif polypeptides or the 3, 4, 5, 6, 7, 8, 9, 10 or 11 processed Nif polypeptides, or both, are functional Nif polypeptides.

In an embodiment, the 3, 4, 5, 6, 7, 8, 9, 10 or 11 Nif polypeptides or preferably the 3, 4, 5, 6, 7, 8, 9, 10 or 11 processed Nif polypeptides, or both, are in mitochondria of the plant cell, preferably in the mitochondrial matrix (MM) of the plant cell.

In an embodiment, the 3, 4, 5, 6, 7, 8, 9, 10 or 11 Nif polypeptides or preferably the 3, 4, 5, 6, 7, 8, 9, 10 or 11 processed Nif polypeptides, or both, are independently predominantly soluble in the plant mitochondria (i.e., greater than 50% soluble in the mitochondria).

In an embodiment, the ND comprises an amino acid other than tyrosine (Y) at a position corresponding to amino acid 100 of SEQ ID NO:18.

In an embodiment, the ND comprises a glutamine (Q) or lysine (K) at the position corresponding to amino acid 100 of SEQ ID NO:18, or a leucine (L) or methionine (M) or a phenylalanine (F) at the position corresponding to amino acid 100 of SEQ ID NO:18.

In an embodiment, the MTP is about 51 amino acids in length from a F1-ATPase γ-subunit MTP.

In an embodiment, the plant cell comprises a NK amino acid sequence, wherein the C-terminus of the polypeptide is a wild-type NifK C-terminus.

In an embodiment, the linker is at least about 20 amino acids, or at least about 30 amino acids, or at least about 40 amino acids, or about 20 amino acids to about 70 amino acids, or about 30 amino acids to about 70 amino acids, or about 30 amino acids to about 60 amino acids, or about 30 amino acids to about 50 amino acids, or about 25 amino acids, or about 30 amino acids, or about 35 amino acids, or about 40 amino acids, or about 45 amino acids, or about 46 amino acids, or about 50 amino acids, or about 55 amino acids, in length.

In an embodiment, the fusion polypeptide is capable of being cleaved within its MTP, or immediately after the MTP, to yield a processed polypeptide (CDK), whereby the CDK comprises in order, an optional C-terminal peptide resulting from cleavage of an MTP, the NifD amino acid sequence (ND), the linker amino acid sequence and the NK amino acid sequence.

In an embodiment, the plant cell further comprises the fusion polypeptide or the CDK, or both.

In an embodiment, the CDK comprises a scar sequence of 1 to 45 amino acids in length, preferably 1 to 20 amino acids, translationally fused at the N-terminal end of the NifD amino acid sequence.

In an embodiment, the CDK has both NifD and NifK function.

In an embodiment, the ND is an AnfD and the NK is an AnfK In an embodiment, the MTP is about 51 amino acids in length from a F1-ATPase γ-subunit MTP.

In an embodiment, each MTP comprises at least 10 amino acids, preferably has a length between 10 and 80 amino acids.

In an embodiment, the MTP, or at least one MTP, or all of the MTPs independently comprise an MTP of a mitochondrial protein precursor, or a variant thereof, preferably a plant MTP.

In an embodiment, the 3, 4, 5, 6, 7, 8, 9, 10 or 11 Nif polypeptides are encoded by 3, 4, 5, 6, 7, 8, 9, 10 or 11 exogenous polynucleotide(s), 3, 4, 5, 6, 7, 8, 9, 10 or 11 of which are integrated into the nuclear genome of the cell, preferably as a contiguous nucleic acid sequence.

In another embodiment of any of the above aspects, the cell is a cell other than an Arabidopsis thaliana protoplast.

The present inventors are the first to produce a plant cell comprising a NifV polypeptide which at least partially soluble in the mitochondria. Thus, in another aspect the present invention provides plant cell comprising a NifV polypeptide (NV), wherein the NV is at least partially soluble in the mitochondria.

In an embodiment, the NV is capable of, or is, producing homocitrate in the cell.

In an embodiment, the NV polypeptide comprises amino acids having a sequence as provided as any one of SEQ ID NO's: 205 to 209, or 211, a biologically active fragment thereof, or has an amino acid sequence which is at least 30% identical, at least 40% identical, at least 50% identical, at least 60% identical, at least 70% identical, at least 80% identical, at least 90% identical, at least 95% identical, at least 97% identical, or at least 99% identical, to that provided in any one or more of SEQ ID NO's: 205 to 209, or 211.

The present inventors are also the first to produce a plant cell comprising a NifH polypeptide which at least partially soluble in the mitochondria. Thus, in another aspect the present invention provides a plant cell comprising a NifH polypeptide (NH), wherein the NH is at least partially soluble in the mitochondria.

In an embodiment, the NH is encoded by an exogenous polynucleotide, one which is integrated into the nuclear genome of the cell, preferably as a contiguous nucleic acid sequence.

In an embodiment, the plant cell of one or both of the above two aspects is further defined by one or more of the features mentioned herein.

In a further aspect, the present invention provides a transgenic plant comprising a plant cell of the invention, wherein the transgenic plant is transgenic for the one or more exogenous polynucleotide(s) encoding the Nif polypeptide(s).

In an embodiment, one, or more of the one or more exogenous polynucleotide(s) are expressed in roots of the plant, preferably expressed at a greater level in the roots of the plant than in leaves of the plant.

In a further or another embodiment, the transgenic plant has an altered phenotype relative to a corresponding wild-type plant which is increased yield, biomass, growth rate, vigor, nitrogen gain derived from biological nitrogen fixation, nitrogen use efficiency, abiotic stress tolerance, and/or tolerance to nutrient deficiency relative to the corresponding wild-type plant.

In an alternative embodiment, the transgenic plant has the same growth rate and/or phenotype relative to a corresponding wild-type plant.

In an embodiment, the plant is a cereal plant such as wheat, rice, maize, triticale, oat or barley, preferably wheat.

In an embodiment, the plant is homozygous or heterozygous for the one or more exogenous polynucleotide(s), preferably homozygous for all of the exogenous polynucleotides.

In another embodiment, the transgenic plant is a monocotyledonous plant such as, for example, a cereal plant such as wheat, rice, maize, triticale, oat, or barley, preferably wheat, or a dicotyledonous plant.

In a further or another embodiment, the transgenic plant is growing in a field.

In a further aspect, the present invention provides a population of at least 100 plants according to the invention growing in a field.

Also provided is a substantially purified or recombinant NifV polypeptide (NV) which when expressed in a plant cell is at least partially soluble in the plant mitochondria.

Further provided is a substantially purified or recombinant NifH polypeptide (NH) which when expressed in a plant cell is at least partially soluble in the plant mitochondria.

In another aspect, provided is an isolated or exogenous polynucleotide encoding a NifV polypeptide (NV), wherein the NV when expressed in a plant cell is at least partially soluble in the plant mitochondria.

In an embodiment, the NV polypeptide comprises amino acids having a sequence as provided as any one of SEQ ID NO's: 205 to 209, or 211, a biologically active fragment thereof, or has an amino acid sequence which is at least 30% identical, at least 40% identical, at least 50% identical, at least 60% identical, at least 70% identical, at least 80% identical, at least 90% identical, at least 95% identical, at least 97% identical, or at least 99% identical, to that provided in any one or more of SEQ ID NO's: 205 to 209, or 211.

In further aspect, provided is an isolated or exogenous polynucleotide encoding a NifH polypeptide (NH) of the invention, wherein the NH when expressed in a plant cell is at least partially soluble in the plant mitochondria.

In an embodiment, a protein coding region of the polynucleotide has been codon-modified for expression in a plant cell, relative to a corresponding protein coding region of a naturally-occurring polynucleotide in a bacterium.

In a further embodiment, the polynucleotide further comprises a promoter operably linked to the polynucleotide and/or translational regulatory elements operably linked to the polynucleotide.

In another embodiment, the promoter confers expression of the one or more polynucleotide(s) in roots, leaves and/or stem of a plant, preferably the promoter confers expression of the one or more polynucleotide(s) in one, or more, or all of the roots, leaves or a stem of the plant relative to seed of the plant.

In another embodiment, the polynucleotide is present in a plant cell or a bacterial cell, preferably integrated into the nuclear genome of the plant cell, for example as a contiguous DNA sequence integrated into the nuclear genome or the chloroplast genome of the plant cell. The plant cell may contain multiple copies of the contiguous DNA sequence integrated into the nuclear genome.

In a further aspect provided is a chimeric vector comprising or encoding a polynucleotide of the invention.

In an embodiment, the polynucleotide, or each sequence within it encoding a polypeptide, is operably linked to a promoter and optionally, a transcription termination sequence.

In a further or another embodiment, the promoter confers expression of one, or more of the polynucleotide(s) in roots, leaves and/or stem of a plant, preferably the one or more polynucleotide(s) is preferentially expressed in one, or more, or all of the roots, leaves or a stem of the plant relative to seed of the plant.

In a further aspect, the present invention provides a cell comprising one, or more, of the polypeptides according to the invention, one, or more of the exogenous polynucleotides according to the invention, and/or a vector according to the invention.

In an embodiment, the cell is a plant cell or a bacterial cell.

In a further embodiment, the plant cell is a monocotyledonous plant cell such as, for example, a cereal plant cell such as a wheat cell, a rice cell, a maize cell, a triticale cell, an oat cell, or a barley cell, preferably a wheat cell, or a dicotyledonous plant cell. In a further aspect, the present invention provides a transgenic plant or a transgenic part thereof, preferably seed, comprising one, or more, of the polypeptides according to the invention, one, or more of the exogenous polynucleotides according to the invention, and/or a vector according to the invention.

In an embodiment, the transgenic plant is a monocotyledonous plant such as, for example, a cereal plant such as wheat, rice, maize, triticale, oat, or barley, preferably wheat, or a dicotyledonous plant.

In a further aspect, the present invention provides a method of producing a polypeptide according to the invention, the method comprising expressing in a cell a polynucleotide according to the invention.

In a further aspect, the present invention provides a method of producing a cell according to the invention, the method comprising the step of introducing one or more polynucleotides according to the invention, and/or a vector according to the invention, into a cell.

In another aspect, the present invention provides a method of producing a transgenic plant, the method comprising the steps of
i) introducing a polynucleotide of the invention, and/or the vector of the invention, into a cell of a plant,
ii) from the cell of step i), regenerating a transgenic plant of the invention, and
iii) optionally, producing one or more transgenic progeny plants from the transgenic plant regenerated in step ii).

In a further aspect, the present invention provides a method of producing transgenic seed, comprising
i) harvesting seed from the transgenic plant of the invention, and/or
ii) harvesting seed from one or more transgenic progeny plants produced by the method of the invention.

In a further aspect, the present invention provides a method of producing a plant which has integrated into its genome a polynucleotide according to the invention, the method comprising the steps of
i) crossing two parental plants, wherein at least one plant comprises the polynucleotide,
ii) screening one or more progeny plants from the cross for the presence or absence of the polynucleotide, and
iii) selecting a progeny plant which comprises the polynucleotide, thereby producing the plant.

In a further or another embodiment, at least one of the parental plants is a tetraploid or hexaploid wheat plant.

In a further or another embodiment, step ii) comprises analysing a sample comprising DNA from the one or more progeny plants for the polynucleotide.

In a further or another embodiment, step iii) comprises
i) selecting a progeny plant which is homozygous for the polynucleotide, and/or
ii) analysing the plant or the one or more progeny plants thereof for presence and/or expression of the polynucleotide or for an altered phenotype as defined above.

In one or a further embodiment, the method further comprises:
iv) backcrossing the progeny of the cross of step i) with a plant of the same genotype as a first parent plant lacking the polynucleotide for a sufficient number of times to produce a plant with a majority of the genotype of the first parent but comprising the polynucleotide, and
iv) selecting a progeny plant which comprises the polynucleotide and/or has an altered phenotype as defined above.

In a further or another embodiment, the method further comprises the step of analysing the plant or progeny plant for at least one other genetic marker.

In a further aspect, the present invention provides a plant produced using a method according to the invention.

In a further aspect, the present invention provides use of a polynucleotide according to the invention, and/or a vector according to the invention, to produce a recombinant cell and/or a transgenic plant.

In an embodiment, the transgenic plant has an altered phenotype as defined above when compared to a corresponding plant lacking the exogenous polynucleotide, and/or the vector.

In a further aspect, the present invention provides a method for identifying a plant comprising a polynucleotide according to the invention, the method comprising the steps of
i) obtaining a nucleic acid sample from a plant, and
ii) screening the sample for the presence or absence of the polynucleotide.

In an embodiment, the presence of the polynucleotide indicates that the plant has an altered phenotype as defined above, when compared to a corresponding plant lacking the exogenous polynucleotide.

In a further or another embodiment, the method identifies a plant according to the invention.

In a further or another embodiment, the method further comprises producing a plant from a seed before step i).

In another aspect, the present invention provides a transgenic plant part comprising a plant cell of the invention or obtained from the transgenic plant of the invention.

In an embodiment, the plant part is a seed that comprises the polynucleotide of the invention.

In another aspect, the present invention provides a method of producing flour, wholemeal, starch, oil, seed meal or other product obtained from seed, the method comprising;
a) obtaining seed of the invention, and
b) extracting the flour, wholemeal, starch, oil or other product, or producing the seed meal.

In a further aspect, the present invention provides a product produced from the transgenic plant of the invention and/or the plant part of the invention comprising the polypeptide of the invention and/or the polynucleotide of the invention.

In an embodiment, the plant part is a seed.

In a further or another embodiment, the product is a food ingredient or beverage ingredient or a food product or beverage product. Preferably, i) the food ingredient or product is selected from the group consisting of: flour, starch, oil, leavened or unleavened breads, pasta, noodles, animal fodder, breakfast cereals, snack foods, cakes, malt, pastries and foods containing flour-based sauces, or ii) the beverage product is juice, beer or malt. Methods of producing such products are well known to those skilled in the art.

In an alternative embodiment, the product is a non-food product. Examples of non-food products include, but are not limited to, films, coatings, adhesives, building materials and packaging materials. Methods of producing such products are well known to those skilled in the art.

In a further aspect, the present invention provides a method of preparing a food product, the method comprising mixing seed of the invention, or flour, wholemeal, starch, oil or other product from the seed, with another food ingredient.

In a further aspect, the present invention provides method of preparing malt, comprising the step of germinating seed according to the invention.

In a further aspect, the present invention provides use of a plant or part thereof according to the invention as animal feed, or to produce feed for animal consumption or food for human consumption.

In a further aspect, the present invention provides a composition comprising a polypeptide according to the invention, a polynucleotide according to the invention, a vector according to the invention, or a cell according to the invention, and one or more acceptable carriers.

Any embodiment herein shall be taken to apply mutatis mutandis to any other embodiment unless specifically stated otherwise. For instance, as the skilled person would understand, examples of Nif polypeptides outlined above for one aspect of the invention equally apply to other aspects the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Western blot analysis using anti-HA antibody to detect individual unprocessed and MPP-processed pFAγ51::Nif::HA or 6×HIS::Nif::HA polypeptides after transient expression in Nicotiana benthamiana leaves. C, cytoplasmic expression (6×His); M, mitochondrially targeted.

FIG. 2. Western blots of protein extracts after introduction of MTP:Nif genetic constructs into N. benthamiana leaf cells. The first and last lanes on each blot show indicative molecular weight markers in kDa from the Invitrogen Prestained BenchMark ladder. The genetic construct(s) used for each sample is indicated above each lane and the Nif polypeptide included in each fusion polypeptide is indicated below the lanes. For constructs SN26-SN32, paired infiltrations were carried out either with or without co-infiltration of pRA25 which encodes a MTP-FAγ77::NifK fusion polypeptide (WO2018/141030). The Western blots were probed with HA-antibody.

FIG. 3. Western blot analysis using anti-HA antibody of individual MTP-FAγ51::Nif::HA polypeptides (with the exception of MTP-FAγ51::HA::NifK) and MPP-processed products thereof after expression in Nicotiana benthamiana leaf cells. T, total protein; I, insoluble fraction; S, soluble fraction.

FIG. 4. Upper panel shows a schematic of the genetic constructs tested for production of a secondary cleavage product from wild-type NifD fusion polypeptides. MTP was either the FAγ51 or the L29 sequence, NifD was the wild-type K. oxytoca sequence, and HA=HA epitope. Lower panel shows a Western blot of protein extracts after introduction of the genetic constructs into N. benthamiana leaf cells. The Western blot was probed with HA-antibody. Lane 1 shows molecular weight markers using Prestained Benchmark ladder. Paired lanes show either the absence (−) or presence (+) of the NifK construct pRA25. Band 1=unprocessed MTP::NifD fusion polypeptide, band 2=MPP-processed fusion polypeptide and band 3 is the ˜48 kDa degradation product.

FIG. 5. Western blot of protein extracts after introduction of MTP:NifD genetic constructs into N. benthamiana leaf cells. Lane 1 shows molecular weight markers in kDa, using ThermoFisher Prestained Benchmark ladder. The genetic construct used in each sample is indicated above each lane. pRA24 encoded a MTP-FAγ::NifD::HA polypeptide where the NifD coding region was codon optimised for Arabidopsis (WO2018/141030). Each construct was introduced into the plant cells together with pRA25 (MTP-FAγ77::NifK) to enhance the NifD fusion polypeptide accumulation. The Western blot was probed with HA-antibody. The arrow shows the position of the ˜48 kDa secondary cleavage polypeptide from NifD.

FIG. 6. Western blot of protein extracts after introduction of MTP:NifD genetic constructs into N. benthamiana leaf cells. Lane 1 shows molecular weight markers in kDa using ThermoFisher Prestained Benchmark ladder. The genetic construct used in each sample is indicated above each lane. SN64 encoded a mMTP-CPN60::NifD polypeptide where the mMTP-CPN60 amino acid sequence had been altered with substitution of amino acids with alanines, thereby rendering it resistant to cleavage by MPP. pRA24 encoded a MTP-FAγ::NifD::HA polypeptide where the NifD coding region was codon optimised for Arabidopsis (WO2018/141030). The Western blot was probed with HA-antibody.

FIG. 7. Alignment of the mutant mMTP-FAγ51 amino acid sequence (SEQ ID NO:59) in SN66 with the unmodified MTP-FAγ51 sequence (SEQ ID NO:21) in SN10 (SEQ ID NO:122). Regions of 5 and 8 consecutive amino acid residues were substituted with alanines, to inactivate MPP processing.

FIG. 8. Western blot of protein extracts after introduction of MTP:Nif genetic constructs into plant cells or yeast cells, probed with HA-antibody, demonstrating NifD secondary cleavage/degradation in yeast cells and reduction of cleavage with a Y100Q amino acid substitution (SN114, SNY114). Protein extracts from N. benthamiana leaf cells (SN10, SN196, SN114) or from yeast (SNY10, SNY196, SNY114) were electrophoresed in the lanes as indicated. Lanes 1 and 8 show molecular weight markers in kDa, using ThermoFisher Prestained Benchmark ladder. The band at ˜64 kDa represents unprocessed MTP::NifD::HA fusion polypeptide, the band at ˜58 kDa represents MPP-processed fusion polypeptide. The arrow points to the ˜48 kDa C-terminal polypeptide produced by the secondary cleavage.

FIG. 9. Western blot of protein extracts from N. benthamiana leaf cells after introduction of genetic constructs encoding MTP::NifD::HA amino acid substitution variants, each together with SN46 (MTP-Su9::NifK). Lane 12 shows molecular weight markers in kDa using ThermoFisher Prestained Benchmark ladder. The most intense band at ˜58 kDa in lanes 5-11 was MPP-processed MTP-FAγ51::NifD. Lanes 2 and 3 show the 48 kDa polypeptide produced by secondary cleavage. Note the absence of the 48 kDa polypeptide in lanes 5-11.

FIG. 10. Amino acid sequence alignment of a region of wild-type NifD polypeptides corresponding to amino acids 49-108 of K. oxytoca NifD (SEQ ID NO:18). A representative sequence was chosen from each cluster that contained at least 10 members in the sequence similarity network. The number of members in each cluster of NifD sequences is shown in parentheses. Completely conserved amino acids are shown above the alignment.

FIG. 11. Location of the proposed secondary cleavage site shown in the crystal structure of the NifD polypeptide from K. oxytoca (PDB:1QGU). Cofactor FeMoco is shown as spheres to the right. NifK-Ser515, NifK-Asp517, C-terminus and the structures to the top left are from NifK polypeptide. Arg97, Arg98, Asn99, Tyr100, Tyr101, Thr102 and structures to the lower right aside from FeMoco are from NifD. Dashed lines indicate possible hydrogen bonds between the hydroxyl of Tyr100 and Ser515, Asp517 and Arg98.

FIG. 12. Western blot analysis showing mitochondrial processing of NifD fusion polypeptides from six different bacteria. Three constructs, in adjacent lanes, were analysed for each NifD sequence: encoding an mMTP-FAγ51::NifD::HA fusion polypeptide which was not cleaved by the MPP at the canonical MPP cleavage site (lanes marked A), MTP-FAγ51::NifD::HA, which was targeted to mitochondria (lanes marked M), and 6×His::NifD::HA, which was expected to be cytoplasmically located (lanes marked C) and corresponding in size to the MPP-processed size.

FIG. 13. Schematic maps of genetic constructs encoding NifD::linker(HA)::NifK fusion polypeptides, not drawn to scale. mMTP-FAγ refers to the mutant MTP having alanine substitutions to prevent cleavage by MPP. Y100Q refers to the presence of the amino acid substitution in the NifD sequence.

FIG. 14. Solubility of NifD-linker(HA)-NifK polypeptides after expression in N. benthamiana. Proteins from infiltrated leaf samples were isolated as “Total” protein or fractionated into Insoluble and Soluble fractions as described in Example 1. The protein ladder marker shown the ThermoFisher Prestained Benchmark ladder was used in blots for ‘Total’ and ‘Insoluble’ samples and the Invitrogen PageRuler ladder was used in the blot for the ‘Soluble’ samples.

FIG. 15. Schematic of a metaxin fusion polypeptide encoded by a gene on SN197 and its localisation in the outer membrane of mitochondria with most of the polypeptide from the N-terminus into the cytoplasm. This construct used the N. benthamiana metaxin sequence.

FIG. 16: Western blot showing that purification of mitochondrially targeted MTP-FAγ51::NifU::TS from SN166 resulted in purification of a processed form of the NifU polypeptide. Upper panel: probed with anti-Strep antibody. Lower panel: Coomassie blue stained gel.

FIG. 17. Western blot showing that purification of mitochondrially targeted scar9::GG::NifU::TS resulted in co-purification of scar9::GG::NifS::HA. Samples from steps (i) to (v) in the purification process of the first purification experiment were subjected to SDS-PAGE and Western blotting using either anti-Strep antibody to detect the NifU polypeptide or anti-HA antibody to detect the NifS polypeptide. The two bands for NifS correspond to the unprocessed and processed forms. The presence of the processed NifS form in the eluate showed that co-purification had occurred.

FIG. 18. Western blot of the purification of NifU from N. benthamiana in a third purification experiment, showing that NifS co-purifies with NifU. Panel A) Schematic of constructs that were infiltrated into N. benthamiana (not drawn to scale). B) Western blot analysis of the purification. P=pellet, S=supernatant, FT=flow through and E=eluate. All samples were loaded in duplicate and subjected to immuno-detection using either a strep-antibody (α-strep) or a HA-antibody (α-HA). C) Coomassie stain of the eluate, which shows a major band for NifU and a faint band for NifS.

FIG. 19. Western blot showing that purification of mitochondrially targeted MTP-FAγ51::NifS::TS resulted in co-purification of scar9::GG::NifU::HA. Samples from steps (ii) to (v) were subjected to SDS-PAGE and Western blotting using either anti-Strep antibody to detect the NifS polypeptide or anti-HA antibody to detect the NifU polypeptide. The two bands for NifS correspond to the unprocessed and processed forms. The presence of the processed NifU form in the eluate showed that co-purification had occurred.

FIG. 20. ClustalW alignment of the first 300 amino acid residues of selected NifV/HCS-like amino acid sequences in this study along with N. benthamiana P72026 (SEQ ID NO:221) and P20586 (SEQ ID NO:222) translation, K. oxytoca NifV (SEQ ID NO:13), Lotus japonicus FEN1 (SEQ ID NO:215), and Mycobacterium tuberculosis α-isopropylmalate synthase (MtLeuA, SEQ ID NO:223). Other HCS sequences are from Thermoanaerobacter brockii (TbHCS; SEQ ID NO:206), Thermincola potens (TpHCS; SEQ ID NO:207), Saccharomyces cerevisiae (ScHCS; SEQ ID NO:208), Nodularia spumigena (NsHCS; SEQ ID NO:209), Methanosarcina acetivorans (MaHCS; SEQ ID NO:210), Chlorobaculum tepidum (CtHCS; SEQ ID NO:211) and Methanocaldococcus infernus (MiHCS1, SEQ ID NO:212; MiHCS2, SEQ ID NO:213; MiHCS, SEQ ID NO:214). Conserved residues in the active site of LeuA are identified by *. The four amino acid residues at positions R81, D82, H291, H293 hold Zn²⁺, and the two amino acid residues E224, T260, along with Zn²⁺ in its position forms the substrate binding pocket of MtLeuA (Koon et al., 2004).

FIG. 21. Western blot analysis using anti-HA antibody of total, insoluble and soluble fractions of NifV/HCS-like fusion polypeptides (MTP-FAγ51::HA::NifV/HCS) after expression in N. benthamiana leaves. T, total protein; I, insoluble (pellet) fraction of total protein; S, soluble (supernatant) fraction of total protein. m, mitochondrial-targeted polypeptide; c, cytoplasmically-targeted polypeptide.

FIG. 22. Western blot analysis using anti-HA antibody of total, insoluble and soluble fractions of cytoplasmically-localised NifV/HCS-like fusion polypeptides (HA::NifV/HCS) after expression in N. benthamiana leaves, used as comparators for the corresponding mitochondrially-localised fusion polypeptides. T, total protein; I, insoluble (pellet) fraction of total protein; S, soluble (supernatant) fraction of total protein. c, cytoplasmically-targeted polypeptide; m, mitochondrial-targeted polypeptide.

FIG. 23. Homocitrate target ion peak area after baseline subtraction (Login scale)

FIG. 24: Western blot analysis of the solubility of NifH fusion polypeptides in a transient leaf expression system in N. benthamiana leaves, using anti-Strep antibody to detect polypeptides having the TwinStrep epitope. All of the NifH genetic constructs were co-infiltrated with SN44 encoding a NifM fusion polypeptide from K. oxytoca. Protein samples were prepared under aerobic conditions.

FIG. 25. Western blot showing the results of purification of a NifH fusion polypeptide encoded by SL6 in stably transformed tobacco. The NifH gene encoded a MTP-CoxIV::TwinStrep::KoNifH::HA fusion polypeptide. Samples of 5 μL from stages in the purification process were analysed by Western blot and probed with antibodies recognising either the Strep or HA epitopes. Samples from the total, insoluble and soluble fractions are indicated above the lanes. Closed arrowheads indicate unprocessed NifH polypeptide, black arrowheads indicate the processed form.

FIG. 26. Western blot analysis of the expression and processing of Anf fusion polypeptides after transient introduction of genetic constructs in N. benthamiana leaves. The blot had sets of three adjacent lanes for (left to right) AnfD, AnfK, AnfH and AnfG fusion polypeptides. Each set included the test fusion polypeptide MTP-FAγ51::HA::Anf and the two control polypeptides HA::Anf and mFAγ51::HA::Anf as molecular weight markers. L, Ladder of molecular weight markers (kDa).

FIG. 27. Western blot showing expression and processing of all four of the AnfD, AnfK, AnfH and AnfG fusion polypeptides when expressed from multi-gene constructs in N. benthamiana leaves. A. Western blot analysis of mitochondrially-targeted AnfD, AnfK, AnfG and AnfH fusion polypeptides expressed from SL26 and unprocessed polypeptides from SL31, detected in total protein extracts from the transient leaf assay. B. Western blot analysis of proteins resulting from expression of mitochondrially-targeted AnfD, AnfK, AnfG and AnfH fusion polypeptides from SL26, and unprocessed fusion polypeptides from SL31. C. Western blot showing expression and processing of fusion polypeptides from the multigene constructs SL26, SL27 and SL28, the single gene construct SL29, and a mixture (Mix) of the four single gene constructs SN161, SN129, SN130 and SN131. When present, AnfK showed an upper, unprocessed band and a lower, processed band.

FIG. 28. Western blot showing solubility of individual Anf polypeptides expressed from single gene vectors in N. benthamiana leaf cells when localised to the cytoplasm or mitochondria. Upper panels, soluble fractions for the AnfD, AnfK, AnfH and AnfG fusion polypeptides; lower panel, insoluble fractions for the AnfD, AnfK, AnfH and AnfG fusion polypeptides. C, cytoplasmic localisation; M, mitochondrial localisation; A, alanine substituted mMTP-FAγ51. Black arrowheads indicate the positions of the MPP-cleaved proteins, open arrowheads the unprocessed polypeptides. See Table 20 for the predicted molecular weights of each Anf in the unprocessed and MPP-processed polypeptides.

FIG. 29. Homology model of the AnfDKHG complex for the Fe-nitrogenase, based on the A. vinelandii Anf amino acid sequences with a linker joining the AnfD and AnfK polypeptides. Initial coordinates prior to the 20 ns simulation. The predicted structure of the AnfD::Linker::AnfK polypeptide, using a 16-amino acid linker, was complexed with AnfH dimers and AnfG. The dimer of AnfH is annotated as AnfHH.

FIG. 30. Western blot analysis of total protein extracts from N. benthamiana leaves infiltrated with genetic constructs for expression of AnfD and AnfK polypeptides, either fused or separate. The blot was probed with anti-HA antibody. The expression of AnfD-linker-AnfK fusion polypeptides from SN272-SN275 was compared to the expression from separate genes on the vectors SL26 and SL28. SN161 and SN129 provided the controls for the expression individually of AnfD and AnfK, respectively.

FIG. 31. Western blot analysis of (A) soluble and (B) insoluble fractions of proteins from N. benthamiana leaves infiltrated with genetic constructs for expression of AnfD and AnfK genes. SN272-SN275 each encoded AnfD-linker-AnfK fusion polypeptides whereas SL26 and SL28 expressed separate polypeptides.

FIG. 32. Western blot analysis of polypeptides produced from SL42 in N. benthamiana leaves, including total (T), insoluble (I) and soluble (S) fractions using the anti-HA (panel A) or anti-Strep antibody (panel B) for detection. Black arrowheads indicate the positions of the processed polypeptide bands after mitochondrial cleavage by MPP, white arrowheads indicate the bands for the unprocessed polypeptides. Panel B probed with the anti-Strep antibody shows the processed NifB polypeptide.

FIG. 33. Western blot analysis of polypeptides produced from SL43 in N. benthamiana leaves, including total (T), insoluble (I) and soluble (S) fractions using the anti-HA (panel A) or anti-Strep antibody (panel B) for detection. Black arrowheads indicate the positions of the processed polypeptide bands after mitochondrial cleavage by MPP, white arrowheads indicate the bands for the unprocessed polypeptides. Panel B probed with the anti-Strep antibody shows the processed AnfK polypeptide.

FIG. 34. Western blot analysis of polypeptides produced from SL42 and SL43 introduced together into N. benthamiana leaves, including total (T), insoluble (I) and soluble (S) fractions using the anti-HA (panel A) or anti-Strep antibody (panel B) for detection. The numbers to the side of panel A) and B) indicate the molecular weights (kDa) of the markers in the first lane. Black arrowheads indicate the positions of the processed polypeptide bands after mitochondrial cleavage by MPP, white arrowheads indicate the bands for the unprocessed polypeptides.

FIG. 35. Western blot analysis of polypeptides produced from SL48 in N. benthamiana leaves, including total (T), insoluble (I) and soluble (S) fractions using the anti-HA (panel A) or anti-Strep antibody (panel B) for detection. The numbers to the side of panel A) and B) indicate the molecular weights (kDa) of the markers in the first lane. Black arrowheads indicate the positions of the processed polypeptide bands after mitochondrial cleavage by MPP, white arrowheads indicate the bands for the unprocessed polypeptides. Panel B probed with the anti-Strep antibody shows the processed NifB polypeptide.

FIG. 36. Western blot analysis of polypeptides produced from SL49 in N. benthamiana leaves, including total (T), insoluble (I) and soluble (S) fractions using the anti-HA (panel A) or anti-Strep antibody (panel B) for detection. Black arrowheads indicate the positions of the processed polypeptide bands after mitochondrial cleavage by MPP, white arrowheads indicate the bands for the unprocessed polypeptides. Panel B probed with the anti-Strep antibody shows the processed AnfK polypeptide.

FIG. 37. Western blot analysis of polypeptides produced from SL48 and SL49 introduced together into N. benthamiana leaves, including total (T), insoluble (I) and soluble (S) fractions using the anti-HA (panel A) or anti-Strep antibody (panel B) for detection. Black arrowheads indicate the positions of the processed polypeptide bands after mitochondrial cleavage by MPP, white arrowheads indicate the bands for the unprocessed polypeptides.

FIG. 38. Western blot analysis of polypeptides produced from SN292, SN291, SN299 and SN300 in N. benthamiana leaves, including total, panel A), insoluble, panel B), and soluble, panel C), fractions using the anti-HA for detection. The numbers to the side indicate the molecular weights (kDa) of the markers in the first lane. Black arrowheads indicate the positions of the processed polypeptide bands after mitochondrial cleavage, white arrowheads indicate the bands for the unprocessed polypeptides, the * indicates a potential dimer of the FdxN protein.

FIG. 39. Western blot analysis of polypeptides produced from SN192, SL50 and SL54 introduced individually, as well as SL50 and SL54 together into N. benthamiana leaves, including Total (panel A), Soluble (panel B) and Insoluble (panel C) fractions using the anti-HA for detection. Black arrowheads indicate the positions of the processed polypeptide bands after mitochondrial cleavage, white arrowheads indicate the bands for the unprocessed polypeptides.

FIG. 40. Western blot analysis of polypeptides produced from SL50 in N. benthamiana leaves, including total, panel A), insoluble, panel B), and soluble, panel C), fractions using the anti-HA for detection. Black arrowheads indicate the positions of the processed polypeptide bands after mitochondrial cleavage, white arrowheads indicate the bands for the unprocessed polypeptides.

FIG. 41. Western blot analysis of polypeptides produced from SL50 and SL49 in N. benthamiana leaves, including total, panel A), insoluble, panel B), and soluble, panel C), fractions using the anti-HA for detection. Black arrowheads indicate the positions of the processed polypeptide bands after mitochondrial cleavage, white arrowheads indicate the bands for the unprocessed polypeptides.

FIG. 42. Western blot analysis of polypeptides produced from SL47 and SL55, separately or in combination, in N. benthamiana leaves using anti-HA for detection. The first lane shows molecular weights (kDa) markers. Black arrowheads indicate the positions of the processed polypeptide bands after mitochondrial cleavage by MPP, white arrowheads indicate the bands for the unprocessed polypeptides.

FIG. 43. Western blot of proteins extracted from leaf samples of transgenic Arabidopsis plants transformed with SL49, probed with anti-HA antibody. Positions of the NifJ, NifB, NifU and NifF fusion polypeptides are indicated by arrows, based on the positions of the same polypeptides after transient expression of SL49 in N. benthamiana leaves (Benth control).

### KEY TO THE SEQUENCE LISTING

SEQ ID NO:1 Amino acid sequence of NifH polypeptide from K. oxytoca, 293aa.
SEQ ID NO:2 Amino acid sequence of wild-type NifD polypeptide from K. oxytoca, according to Accession No. X13303.1; 483aa (The Temme sequence is SEQ ID NO:18).
SEQ ID NO:3 Amino acid sequence of NifK polypeptide from K. oxytoca, according to Temme et al. (2012); 520aa.
SEQ ID NO:4 Amino acid sequence of NifB polypeptide from K. oxytoca, 468aa.
SEQ ID NO:5 Amino acid sequence of NifE polypeptide from K. oxytoca, 457aa.
SEQ ID NO:6 Amino acid sequence of NifF polypeptide from K. oxytoca, 176 aa; NCBI Accession No. X03214.
SEQ ID NO:7 Amino acid sequence of NifJ polypeptide from K. oxytoca, 1171 aa; NCBI Accession No. 43862.; Cannon et al., 1988 Nucleic Acids Res. 16:11379).
SEQ ID NO:8 Amino acid sequence of NifM polypeptide from K. oxytoca, 266 aa; NCBI Accession No. X05887; Paul and Merrick (1987).
SEQ ID NO:9 Amino acid sequence of NifN polypeptide from K. oxytoca, NCBI Accession No. P08738; 461aa; (Arnold et al., 1988). This sequence is identical to a K. michiganensis sequence Accession No. WP_064371582 and is 85% identical to a sequence annotated as K. oxytoca NifN, Accession No. WP_061153953.
SEQ ID NO:10 Amino acid sequence of NifQ polypeptide from Klebsiella. NCBI Accession No. WP_004138772. This sequence is 95% identical to another K. oxytoca sequence annotated as NifQ, Accession No. AAA25108.1.
SEQ ID NO:11 Amino acid sequence of NifS polypeptide from K. oxytoca, 400aa.
SEQ ID NO:12 Amino acid sequence of NifU polypeptide from K. oxytoca; 274aa. NCBI Accession No. P05343.2 (Arnold et al., 1988). This sequence is identical to Accession No. WP_004138782 and also is 272/273 identical to another K. oxytoca sequence, Accession No. AAA25155.
SEQ ID NO:13 Amino acid sequence of NifV polypeptide from K. oxytoca; 381aa. NCBI Accession No. CAA31119.1 (Arnold et al., 1988).
SEQ ID NO:14 Amino acid sequence of NifX polypeptide from K. oxytoca, 156aa (Accession No. P09136).
SEQ ID NO:15 Amino acid sequence of NifY polypeptide from K. oxytoca, 220aa; NCBI Accession No. CAA31670 (Arnold et al., 1988).
SEQ ID NO:16 Amino acid sequence of NifZ polypeptide from K. oxytoca, 148aa; NCBI Accession No. P0A3U2 (Arnold et al., 1988).
SEQ ID NO:17. Amino acid sequence of NifW polypeptide from K. oxytoca.
SEQ ID NO:18. Amino acid sequence of wild-type K. oxytoca NifD according to Temme et al. (2012).
SEQ ID NO:19. Amino acid sequence of wild-type K. oxytoca NifS according to Temme et al. (2012).
SEQ ID NO:20. Amino acid sequence of the N-terminal extension comprising the MTP-FAγ77 (amino acids 1-77) and the amino acid triplet GAP (78-80). Cleavage by MPP occurs between amino acid residues 42 and 43.
SEQ ID NO:21. Amino acid sequence of the MTP-FAγ51 polypeptide with additional N-terminal Met and C-terminal GG. Cleavage by MPP occurs between amino acid residues 43 and 44.
SEQ ID NO:22. Amino acid sequence of the FAγ-scar9 polypeptide.
SEQ ID NO:23. Amino acid sequence of the MTP-FAγ77::NifH::HA fusion polypeptide encoded by pRA10. Amino acids 1-77 correspond to MTP-FAγ77, amino acids 78-80 are the GAP, amino acids 81-372 correspond to K. oxytoca NifH amino acids (SEQ ID NO:1 without the initiator Met) and amino acids 373-389 include the HA epitope.
SEQ ID NO:24. Amino acid sequence of the MTP-FAγ51::NifH::HA fusion polypeptide encoded by pRA34. Amino acids 1-51 correspond to MTP-FAγ51, amino acids 52-54 are the GAP, amino acids 55-346 correspond to K. oxytoca NifH (SEQ ID NO:1 without the initiator Met) and amino acids 347-363 include the HA epitope.
SEQ ID NO:25. Amino acid sequence of the MTP-FAγ51::NifH::HA fusion polypeptide encoded by SN18. Amino acids 1-54 correspond to the MTP-FAγ51 with GG, amino acids 55-347 correspond to K. oxytoca NifH (SEQ ID NO:1) and amino acids 348-358 include the HA epitope.
SEQ ID NO:26. Amino acid sequence of the MTP-FAγ51::HA::NifH fusion polypeptide encoded by SN29. Amino acids 1-53 correspond to the MTP-FAγ51 with GG, amino acids 54-64 include the HA epitope, amino acids 65-357 correspond to K. oxytoca NifH
(SEQ ID NO:1), and amino acids 358-371 were a C-terminal extension.
SEQ ID NO:27. 6×His sequence used instead of a MTP sequence, with N-terminal Met and C-terminal GG.
SEQ ID NO:28. Amino acid sequence of the CPN60 MTP.
SEQ ID NO:29. Amino acid sequence of the CPN60/No GGlinker MTP.
SEQ ID NO:30. Amino acid sequence of the Superoxide dismutase (SOD) MTP.
SEQ ID NO:31. Amino acid sequence of the Superoxide dismutase doubled (2SOD) MTP.
SEQ ID NO:32. Amino acid sequence of the Superoxide dismutase, modified (SODmod) MTP.
SEQ ID NO:33. Amino acid sequence of the Superoxide dismutase, modified (2SODmod) doubled MTP.
SEQ ID NO:34. Amino acid sequence of the L29 MTP (At1G07830).
SEQ ID NO:35. Amino acid sequence of the Neurospora crassa F0 ATPase subunit 9 (SU9) MTP.
SEQ ID NO:36. Amino acid sequence of the gATPase gamma subunit (FAγ51) MTP, without the additional N-terminal Met (SEQ ID NO:21 has an additional N-terminal Met). Cleavage by MPP occurs between amino acid residues 42 and 43.
SEQ ID NO:37. Amino acid sequence of the CoxIV twin strep (ABM97483) MTP.
SEQ ID NO:38. Amino acid sequence of the CoxIV 10×His (ABM97483) MTP.
SEQ ID NO:39. Amino acid sequence of the predicted scar for the Superoxide dismutase (SOD) MTP with GG and for the Superoxide dismutase, doubled (2SOD) MTP with GG.
SEQ ID NO:40. Amino acid sequence of the predicted scar for the L29 MTP with GG.
SEQ ID NO:41. Amino acid sequence of the predicted scar for the Neurospora crassa F0 ATPase subunit 9 (SU9) MTP with GG.
SEQ ID NO:42. Amino acid sequence of the predicted scar for the gATPase gamma subunit (FAγ51) MTP with GG.
SEQ ID NO:43. Amino acid sequence of the predicted scar for the CoxIV twin strep MTP with GG.
SEQ ID NO:44. Amino acid sequence of the predicted scar for the CoxIV 10×His MTP with GG.
SEQ ID NO:45. Oligonucleotide primer MIT_V2.1_Sbf1nifH_FW2.
SEQ ID NO:46. Oligonucleotide primer MIT_V2.1_Sbf1nifJ_RV2.
SEQ ID NO:47. Oligonucleotide primer MIT_V2.1_Sbf1nifB_FW.
SEQ ID NO:48. Oligonucleotide primer MIT_V2.1_Sbf1ori_RV.
SEQ ID NO:49. Amino acid sequence of mscar9 from MTP-FAγ51 having substitution of the N-terminal Ile residue with a Met for translation initiation.
SEQ ID NO:50. Tryptic peptide.
SEQ ID NO:51. Amino acid sequence of MTP-FAγ9 scar without N-terminal Met and with C-terminal Met.
SEQ ID NOs:52-54. Oligonucleotide primers.
SEQ ID NO:55. Tryptic peptide.
SEQ ID NO:56. Tryptic peptide.
SEQ ID NO:57. Amino acid sequence of the MTP-FA777::NifK fusion polypeptide (pRA25), lacking any C-terminal extension Amino acids 1-77 correspond to the MTP-FAγ77, amino acids 78-80 are GAP, and amino acids 81-599 correspond to K. oxytoca NifK without the initiator Met.
SEQ ID NO:58. Amino acid sequence of the last four amino acid residues at the C-terminus of the NifK polypeptide from K. oxytoca.
SEQ ID NO:59. Amino acid sequence of the mutant MTP-FAγ51 polypeptide which is not cleaved by MPP.
SEQ ID NOs:60-107. Peptide sequences.
SEQ ID NOs:108-113. Oligonucleotide primers.
SEQ ID NO:114. Amino acid sequence of an 11-residue section from a linker region from Hypocrea jecorina cellobiohydrolase II (Accession no. AAG39980.1).
SEQ ID NO:115. Amino acid sequence of 9-residue HA epitope.
SEQ ID NO:116. Amino acid sequence of a linker for the NifD::linker::NifK fusion polypeptide. The linker is 30 residues in length and has SEQ ID NO:114 with the final arginine replaced by an alanine, then an 9-residue HA epitope (SEQ ID NO:115) followed by another copy of SEQ ID NO:114 with the arginine replaced by an alanine.
SEQ ID NO:117. Oligonucleotide primer.
SEQ ID NO:118. Oligonucleotide primer.
SEQ ID NO:119. Scar peptide sequence.
SEQ ID NO:120. Scar peptide sequence.
SEQ ID NO:121. Amino acid sequence of the metaxin fusion polypeptide encoded by construct SN197. The TwinStrep epitope corresponds to amino acids 1-31, mTurquoise to amino acids 32-273, a TEV cleavage site to amino acids 274-282 and the metaxin sequence to amino acids 283-603.
SEQ ID NO:122 Amino acid sequence of the MTP-FAγ51::NifD::HA fusion polypeptide encoded by SN10. Amino acids 1-54 correspond to the MTP-FAγ51 with GG at its C-terminus, amino acids 55-536 correspond to K. oxytoca NifD (SEQ ID NO:18) with its initiator Met, and amino acids 537-547 include the HA epitope.
SEQ ID NO:123. Amino acid sequence of the MTP-FAγ51::NifM::HA fusion polypeptide encoded by SN30. Amino acids 1-54 correspond to the MTP-FAγ51 with GG at its C-terminus, amino acids 55-320 correspond to K. oxytoca NifM (SEQ ID NO:8) with its initiator Met, and amino acids 321-331 include the HA epitope.
SEQ ID NO:124. Amino acid sequence of the MTP-FAγ51::NifS::HA fusion polypeptide encoded by SN31. Amino acids 1-54 correspond to the MTP-FAγ51 with GG at its C-terminus, amino acids 55-454 correspond to K. oxytoca NifS (SEQ ID NO:19) with its initiator Met, according to Temme et al. (2012), and amino acids 455-465 include the HA epitope.
SEQ ID NO:125. Amino acid sequence of the MTP-FAγ51::NifU::HA fusion polypeptide encoded by SN32. Amino acids 1-54 correspond to the MTP-FAγ51 with GG at its C-terminus, amino acids 55-328 correspond to K. oxytoca NifU (SEQ ID NO:12) with its initiator Met, and amino acids 329-339 include the HA epitope.
SEQ ID NO: 126. Amino acid sequence of the MTP-FAγ51::NifE::HA fusion polypeptide encoded by SN38. Amino acids 1-54 correspond to the MTP-FAγ51 with GG at its C-terminus, amino acids 55-511 correspond to K. oxytoca NifE with its initiator Met according to Temme et al. (2012), and amino acids 512-522 include the HA epitope.
SEQ ID NO:127. Amino acid sequence of the MTP-FAγ51::NifN::HA fusion polypeptide encoded by SN39. Amino acids 1-54 correspond to the MTP-FAγ51 with GG at its C-terminus, amino acids 55-515 correspond to K. oxytoca NifN (SEQ ID NO:9) with its initiator Met, and amino acids 516-526 include the HA epitope.
SEQ ID NO:128. Amino acid sequence of the MTP-CoxIV-Twin-Strep::NifH::HA fusion polypeptide encoded by SN42. Amino acids 1-61 correspond to the MTP-CoxIV-Twin-Strep with GG at its C-terminus, amino acids 62-354 correspond to K. oxytoca NifH amino acids (SEQ ID NO:1) with its initiator Met, and amino acids 355-365 include the HA epitope.
SEQ ID NO: 129. Amino acid sequence of the MTP-Su9::NifK fusion polypeptide encoded by SN46. Amino acids 1-70 correspond to the MTP-Su9 with GG at its C-terminus, amino acids 71-590 correspond to K. oxytoca NifK (SEQ ID NO:3) with its initiator Met.
SEQ ID NO:130. Amino acid sequence of the MTP-L29::NifV::HA fusion polypeptide encoded by SN51. Amino acids 1-34 correspond to the MTP-L29 with GG at its C-terminus, amino acids 35-415 correspond to K. oxytoca NifV (SEQ ID NO:13) with its initiator Met, and amino acids 416-426 include the HA epitope.
SEQ ID NO:131. Amino acid sequence of the MTP-FAγ51::NifD::linker(HA)::NifK fusion polypeptide encoded by SN68. Amino acids 1-54 correspond to the MTP-FAγ51 with GG at its C-terminus, amino acids 55-536 correspond to wild-type K. oxytoca NifD amino acids (SEQ ID NO:18 without N-terminal Met), amino acids 537-566 correspond to the linker including the HA epitope, and amino acids 567-1085 correspond to NifK
(SEQ ID NO:3) without its N-terminal Met and with its wild-type C-terminus.
SEQ ID NO:132. Amino acid sequence of the MTP-FAγ51::HA::NifD::HA fusion polypeptide encoded by SN75. Amino acids 1-53 correspond to the MTP-FAγ51 with GG at its C-terminus, amino acids 54-64 correspond to the first HA epitope, amino acids 65-546 correspond to wild-type K. oxytoca NifD amino acids (SEQ ID NO:18), and amino acids 547-557 include the HA epitope.
SEQ ID NO:133. Amino acid sequence of the MTP-FAγ51::NifD::HA fusion polypeptide encoded by SN99. Amino acids 1-54 correspond to the MTP-FAγ51 with GG at its C-terminus, amino acids 55-536 correspond to K. oxytoca NifD comprising the alanine substitution mutations at amino acids 148-152, and amino acids 537-547 include the HA epitope.
SEQ ID NO:134. Amino acid sequence of the MTP-FAγ51::NifD::HA fusion polypeptide encoded by SN100. Amino acids 1-54 correspond to the MTP-FAγ51 with GG at its C-terminus, amino acids 55-536 correspond to K. oxytoca NifD amino acids comprising the alanine substitution mutations at amino acids 153-157, and amino acids 537-547 include the HA epitope.
SEQ ID NO:135. Amino acid sequence of the MTP-Su9::NifW fusion polypeptide encoded by SN104. Amino acids 1-70 correspond to the MTP-Su9 with GG at its C-terminus, amino acids 71-158 correspond to K. oxytoca NifW (SEQ ID NO:17) with its initiator Met, and amino acids 159-167 include the HA epitope.
SEQ ID NO:136. Amino acid sequence of the MTP-FAγ51::NifD::HA fusion polypeptide encoded by SN114. Amino acids 1-54 correspond to the MTP-FAγ51 with GG at its C-terminus, amino acids 55-536 correspond to K. oxytoca NifD comprising the Y100Q substitution mutation at amino acid 154, and amino acids 537-547 include the HA epitope.
SEQ ID NO:137. Amino acid sequence of the MTP-FAγ51::NifF::HA fusion polypeptide encoded by SN138. Amino acids 1-54 correspond to the MTP-FAγ51 with GG, amino acids 55-230 correspond to K. oxytoca NifF (SEQ ID NO:6) and amino acids 231-241 include the HA epitope.
SEQ ID NO:138. Amino acid sequence of the MTP-FAγ51::NifJ::HA fusion polypeptide encoded by SN139. Amino acids 1-54 correspond to the MTP-FAγ51 with
GG, amino acids 55-1225 correspond to K. oxytoca NifJ (SEQ ID NO:7), and amino acids 1226-1236 include the HA epitope.
SEQ ID NO:139. Amino acid sequence of the MTP-FAγ51::HA::NifK fusion polypeptide encoded by SN140. Amino acids 1-53 correspond to the MTP-FAγ51 with GG, amino acids 54-64 include the HA epitope, and amino acids 65-584 correspond to
K. oxytoca NifK (SEQ ID NO:3) with wild-type C-terminus.
SEQ ID NO:140. Amino acid sequence of the MTP-FAγ51::NifQ::HA fusion polypeptide encoded by SN141. Amino acids 1-54 correspond to the MTP-FAγ51 with GG, amino acids 55-221 correspond to K. oxytoca NifQ (SEQ ID NO:10) and amino acids 222-232 include the HA epitope.
SEQ ID NO:141. Amino acid sequence of the MTP-FAγ51::NifV::HA fusion polypeptide encoded by SN142. Amino acids 1-54 correspond to the MTP-FAγ51 with GG, amino acids 55-435 correspond to K. oxytoca NifV (SEQ ID NO:13) and amino acids 436-446 include the HA epitope.
SEQ ID NO:142. Amino acid sequence of the MTP-FAγ51::NifW::HA fusion polypeptide encoded by SN143. Amino acids 1-54 correspond to the MTP-FAγ51 with GG, amino acids 55-140 correspond to K. oxytoca NifW (SEQ ID NO:17), and amino acids 141-151 include the HA epitope.
SEQ ID NO:143. Amino acid sequence of the MTP-FAγ51::NifX::HA fusion polypeptide encoded by SN144. Amino acids 1-54 correspond to the MTP-FAγ51 with GG, amino acids 55-210 correspond to K. oxytoca NifX (SEQ ID NO:14), and amino acids 211-221 include the HA epitope.
SEQ ID NO:144. Amino acid sequence of the MTP-FAγ51::NifY::HA fusion polypeptide encoded by SN145. Amino acids 1-54 correspond to the MTP-FAγ51 with GG, amino acids 55-274 correspond to K. oxytoca NifY according to Temme et al. (2012), and amino acids 275-285 include the HA epitope.
SEQ ID NO:145. Amino acid sequence of the MTP-FAγ51::NifZ::HA fusion polypeptide encoded by SN146. Amino acids 1-54 correspond to the MTP-FAγ51 with GG, amino acids 55-202 correspond to K. oxytoca NifZ (SEQ ID NO:16), and amino acids 203-213 include the HA epitope.
SEQ ID NO:146. Amino acid sequence of MTP-FAγ51::NifD(Y100Q)::linker(HA)::NifK fusion polypeptide encoded by SN159. Amino acids 1-54 correspond to the MTP-FAγ51 with GG at its C-terminus, amino acids 55-536 correspond to K. oxytoca NifD with the Y100Q substitution, amino acids 537-566 correspond to the linker including the HA epitope, and amino acids 567-1085 correspond to NifK (SEQ ID NO:3) without its N-terminal Met and with its wild-type C-terminus.
SEQ ID NO:147. Amino acid sequence of the MTP-FAγ51::NifB::HA fusion polypeptide encoded by SN192. Amino acids 1-54 correspond to the MTP-FAγ51 with GG, amino acids 55-522 correspond to K. oxytoca NifB according to Temme et al. (2012), and amino acids 523-533 include the HA epitope.
SEQ ID NO:148. Amino acid sequence of wild-type Azospirillum brasilense NifD polypeptide, UniProt A0A060DN91; 479aa.
SEQ ID NO:149. Amino acid sequence of wild-type Azotobacter vinelandii NifD polypeptide, UniProt C1DGZ7; 492aa.
SEQ ID NO:150. Amino acid sequence of wild-type Sinorhizobium fredii NifD polypeptide, 504aa.
SEQ ID NO:151. Amino acid sequence of wild-type Chlorobium tepidum NifD polypeptide, Uniprot Q8KC89; 543aa.
SEQ ID NO:152. Amino acid sequence of wild-type Desulfovibrio vulgaris NifD polypeptide, Uniprot B8DR77; 544aa.
SEQ ID NO:153. Amino acid sequence of wild-type Desulfotomaculum ferrireducens NifD polypeptide, 539aa.
SEQ ID NO:154. Peptide sequence, where X is any amino acid other than Tyr.
SEQ ID NO:155. Tryptic peptide sequence from NifM.
SEQ ID NO:156. Tryptic peptide sequence from NifM.
SEQ ID NO:157. Tryptic peptide sequence from CAT.
SEQ ID NO:158. Tryptic peptide sequence from CAT.
SEQ ID NO:159. Tryptic peptide sequence from CAT.
SEQ ID NO:160. Amino acid sequence of the MTP-FAγ51::NifU::TwinStrep fusion polypeptide encoded by SN166 Amino acids 1-54 are the MTP-FAγ51 sequence with an additional methionine translational start and C-terminal GG, amino acids 55-328 are the NifU sequence, and amino acids 329-358 are the sequence including a Twinstrep motif.
SEQ ID NO:161. Amino acid sequence of the MTP-FAγ51::NifS::TwinStrep fusion polypeptide encoded by SN231 Amino acids 1-54 are the MTP-FAγ51 sequence with an additional methionine translational start and C-terminal GG, amino acids 55-454 are the NifS sequence, and amino acids 455-484 are the sequence including a Twinstrep motif.
SEQ ID NO:162. Tryptic peptide sequence from scar9.
SEQ ID NO:163. Amino acid sequence of the NifV polypeptide from A. vinelandii (AvNifV; Accession No. WP_012698855).
SEQ ID NO:164. Amino acid sequence of the KoNifV variant sequence (Accession No. WP_004138778).
SEQ ID NO:165. N-terminal ScHCS extension (scar sequence).
SEQ ID NO:166. N-terminal AvNifV extension (scar sequence).
SEQ ID NO:167. Amino acid sequence of the MTP-FAγ51::HA::KoNifM polypeptide encoded by SN43. Amino acids 1-53 correspond to the MTP-FAγ51 sequence including a GG at its C-terminus, amino acids 54-64 correspond to the HA epitope including a GG at its C-terminus, and amino acids 65-330 correspond to the NifM sequence from K. oxytoca.
SEQ ID NO:168. Amino acid sequence of the MTP-CoxIV::TwinStrep::NifH polypeptide encoded by SN178. Amino acids 1-31 correspond to the MTP-CoxIV sequence, amino acids 32-61 correspond to the TwinStrep sequence including a GG at its C-terminus, and amino acids 62-354 correspond to the NifH sequence from Azospirillum brasilense (Accession No. WP_014239786).
SEQ ID NO:169. Amino acid sequence of the MTP-CoxIV::TwinStrep::NifH polypeptide encoded by SN179. Amino acids 1-31 correspond to the MTP-CoxIV sequence, amino acids 32-61 correspond to the TwinStrep sequence including a GG at its C-terminus, and amino acids 62-356 correspond to the NifH sequence from Mastigocladus laminosus (Accession No. WP_016865872).
SEQ ID NO:170. Amino acid sequence of the MTP-CoxIV::TwinStrep::NifH polypeptide encoded by SN180. Amino acids 1-31 correspond to the MTP-CoxIV sequence, amino acids 32-61 correspond to the TwinStrep sequence including a GG at its C-terminus, and amino acids 62-348 correspond to the NifH sequence from Frankia casurinae (Accession No. WP_0011438842).
SEQ ID NO:171. Amino acid sequence of the MTP-CoxIV::TwinStrep::NifH polypeptide encoded by SN181. Amino acids 1-31 correspond to the MTP-CoxIV sequence, amino acids 32-61 correspond to the TwinStrep sequence including a GG at its C-terminus, and amino acids 62-354 correspond to the NifH sequence from Marichromatium gracile biotype thermosufidiphilum (Accession No. WP_062275270).
SEQ ID NO:172. Amino acid sequence of the MTP-CoxIV::TwinStrep::NifH polypeptide encoded by SN182. Amino acids 1-31 correspond to the MTP-CoxIV sequence, amino acids 32-61 correspond to the TwinStrep sequence including a GG at its C-terminus, and amino acids 62-345 correspond to the NifH sequence from Methanocaldococcus infernus (Accession No. WP_013099459).
SEQ ID NO:173. Amino acid sequence of the MTP-CoxIV::TwinStrep::NifH polypeptide encoded by SN183. Amino acids 1-31 correspond to the MTP-CoxIV sequence, amino acids 32-61 correspond to the TwinStrep sequence including a GG at its C-terminus, and amino acids 62-345 correspond to the NifH sequence from Heliobacterium modesticaldum (Accession No. WP_012282218).
SEQ ID NO:174. Amino acid sequence of the MTP-CoxIV::TwinStrep::NifH polypeptide encoded by SN184. Amino acids 1-31 correspond to the MTP-CoxIV sequence, amino acids 32-61 correspond to the TwinStrep sequence including a GG at its C-terminus, and amino acids 62-335 correspond to the NifH sequence from Chlorobium tepidum (Accession No. WP_010933198).
SEQ ID NO:175. Amino acid sequence of the MTP-CoxIV::TwinStrep::NifH polypeptide encoded by SN185. Amino acids 1-31 correspond to the MTP-CoxIV sequence, amino acids 32-61 correspond to the TwinStrep sequence including a GG at its C-terminus, and amino acids 62-350 correspond to the NifH sequence from Geobacter sp. M21 (Accession No. WP_015837436).
SEQ ID NO:176. Amino acid sequence of the MTP-CoxIV::TwinStrep::NifH polypeptide encoded by SN186. Amino acids 1-31 correspond to the MTP-CoxIV sequence, amino acids 32-61 correspond to the TwinStrep sequence including a GG at its C-terminus, and amino acids 62-355 correspond to the NifH sequence from Bradyrhizobium diazoefficans (Accession No. AHY57040).
SEQ ID NO:177. Amino acid sequence of the MTP-CoxIV::TwinStrep::NifH polypeptide encoded by SN187. Amino acids 1-31 correspond to the MTP-CoxIV sequence, amino acids 32-61 correspond to the TwinStrep sequence including a GG at its C-terminus, and amino acids 62-336 correspond to the NifH sequence from Methanobacterium thermoautotrophicum (Accession No. AAB86034).
SEQ ID NO:178. Amino acid sequence of the MTP-CoxIV::TwinStrep::NifH polypeptide encoded by SN188. Amino acids 1-31 correspond to the MTP-CoxIV sequence, amino acids 32-61 correspond to the TwinStrep sequence including a GG at its C-terminus, and amino acids 62-334 correspond to the NifH sequence from Methanosarcina (Accession No. WP_048121466).
SEQ ID NO:179. Amino acid sequence of the MTP-CoxIV::TwinStrep::NifH polypeptide encoded by SN189. Amino acids 1-31 correspond to the MTP-CoxIV sequence, amino acids 32-61 correspond to the TwinStrep sequence including a GG at its C-terminus, and amino acids 62-336 correspond to the NifH sequence from Desulfotomaculum acetoxidans (Accession No. WP_015756624).
SEQ ID NO:180. Amino acid sequence of the MTP-CoxIV::TwinStrep::NifH polypeptide encoded by SN190. Amino acids 1-31 correspond to the MTP-CoxIV sequence, amino acids 32-61 correspond to the TwinStrep sequence including a GG at its C-terminus, and amino acids 62-336 correspond to the NifH sequence from Carboxydothermus pertinax (Accession No. WP_075859892).
SEQ ID NO:181. Amino acid sequence of the MTP-CoxIV::TwinStrep::NifH polypeptide encoded by SN191. Amino acids 1-31 correspond to the MTP-CoxIV sequence, amino acids 32-61 correspond to the TwinStrep sequence including a GG at its C-terminus, and amino acids 62-335 correspond to the NifH sequence from Nostoc calcicole (Accession No. WP_073644321).
SEQ ID NO:182. Amino acid sequence of the MTP-FAγ51::AnfD::HA polypeptide encoded by SN81 Amino acids 1-54 correspond to the MTP-FAγ51 sequence including a GG linker at its C-terminus, amino acids 55-572 correspond to the AnfD sequence from A. vinelandii, and amino acids 573-583 correspond to the HA epitope.
SEQ ID NO:183. Amino acid sequence of the HA::AnfD polypeptide encoded by SN82. Amino acids 1-12 correspond to the HA epitope sequence including a GG linker at its C-terminus, and amino acids 13-530 correspond to the AnfD sequence from A. vinelandii.
SEQ ID NO:184. Amino acid sequence of the MTP-FAγ51::HA::AnfK polypeptide encoded by SN129. Amino acids 1-53 correspond to the MTP-FAγ51 sequence including a GG linker at its C-terminus, amino acids 54-64 correspond to the HA epitope, and amino acids 65-526 correspond to the AnfK sequence from A. vinelandii.
SEQ ID NO:185. Amino acid sequence of the MTP-FAγ51::HA::AnfH polypeptide encoded by SN130. Amino acids 1-53 correspond to the MTP-FAγ51 sequence including a GG linker at its C-terminus, amino acids 54-64 correspond to the HA epitope with a GG linker at its C-terminus, and amino acids 65-339 correspond to the AnfH sequence from A. vinelandii.
SEQ ID NO:186. Amino acid sequence of the MTP-FAγ51::HA::AnfG polypeptide encoded by SN131. Amino acids 1-53 correspond to the MTP-FAγ51 sequence including a GG linker at its C-terminus, amino acids 54-64 correspond to the HA epitope with a GG linker at its C-terminus, and amino acids 65-196 correspond to the AnfG sequence from A. vinelandii.
SEQ ID NO:187. Amino acid sequence of the HA::AnfK polypeptide encoded by SN152 Amino acids 1-12 correspond to the HA epitope sequence including a GG linker at its C-terminus, and amino acids 13-474 correspond to the AnfK sequence from A. vinelandii.
SEQ ID NO:188. Amino acid sequence of the HA::AnfH polypeptide encoded by SN153 Amino acids 1-12 correspond to the HA epitope sequence including a GG linker at its C-terminus, and amino acids 13-287 correspond to the AnfH sequence from A. vinelandii.
SEQ ID NO:189. Amino acid sequence of the HA::AnfG polypeptide encoded by SN154 Amino acids 1-12 correspond to the HA epitope sequence including a GG linker at its C-terminus, and amino acids 13-144 correspond to the AnfG sequence from A. vinelandii.
SEQ ID NO:190. Amino acid sequence of the mFAγ51::HA::AnfK polypeptide encoded by SN155 Amino acids 1-53 correspond to the mutant mFAγ51 sequence including a GG linker at its C-terminus, amino acids 54-64 correspond to the HA epitope with a GG linker at its C-terminus, and amino acids 65-526 correspond to the AnfK sequence from A. vinelandii.
SEQ ID NO:191. Amino acid sequence of the mFAγ51::HA::AnfH polypeptide encoded by SN156 Amino acids 1-53 correspond to the mutant mFAγ51 sequence including a GG linker at its C-terminus, amino acids 54-64 correspond to the HA epitope with a GG linker at its C-terminus, and amino acids 65-339 correspond to the AnfH sequence from A. vinelandii.
SEQ ID NO:192. Amino acid sequence of the mFAγ51::HA::AnfG polypeptide encoded by SN157 Amino acids 1-53 correspond to the mutant mFAγ51 sequence including a GG linker at its C-terminus, amino acids 54-64 correspond to the HA epitope with a GG linker at its C-terminus, and amino acids 65-196 correspond to the AnfG sequence from A. vinelandii.
SEQ ID NO:193. Amino acid sequence of the mFAγ51::HA::AnfD polypeptide encoded by SN158 Amino acids 1-53 correspond to the mutant mFAγ51 sequence including a GG linker at its C-terminus, amino acids 54-64 correspond to the HA epitope with a GG linker at its C-terminus, and amino acids 65-582 correspond to the AnfD sequence from A. vinelandii.
SEQ ID NO:194. Amino acid sequence of the MTP-FAγ51::HA::AnfD polypeptide encoded by SN161. Amino acids 1-53 correspond to the MTP-FAγ51 sequence including a GG linker at its C-terminus, amino acids 54-64 correspond to the HA epitope with a GG linker at its C-terminus, and amino acids 65-582 correspond to the AnfD sequence from A. vinelandii.
SEQ ID NO:195. Amino acid sequence of the MTP-FAγ51::AnfD::Twin Strep polypeptide encoded by SN177 Amino acids 1-54 correspond to the MTP-FAγ51 sequence including a GG linker at its C-terminus, amino acids 55-572 correspond to the AnfD sequence from A. vinelandii, and amino acids 573-604 correspond to the TwinStrep epitope.
SEQ ID NO:196. Amino acid sequence of the MTP-CoxIV::Twin Strew AnfK polypeptideencoded by SN195. Amino acids 1-41 correspond to the MTP-CoxIV sequence including a GG linker at its C-terminus, amino acids 42-61 correspond to the TwinStrep epitope including a GG at the C-terminus, and amino acids 62-523 correspond to the AnfK sequence from A. vinelandii.
SEQ ID NO:197. Peptide sequence.
SEQ ID NO:198. Linker sequence.
SEQ ID NO:199. Amino acid sequence of AnfD::linker16::AnfK polypeptide used for modelling the structure (Example 20) Amino acids 1-509 correspond to the AnfD sequence (A. vinelandii) omitting the N-terminal methionine, amino acids 510-525 correspond to the 16-amino acid linker, and amino acids 526-984 to AnfK (A. vinelandii).
SEQ ID NO:200. Linker sequence.
SEQ ID NO:201. Amino acid sequence of AnfD::linker26(HA)::AnfK polypeptide. Amino acids 1-517 correspond to the AnfD sequence, amino acids 518-543 correspond to the 26-amino acid linker, and amino acids 544-1004 to AnfK.
SEQ ID NO:202. Amino acid sequence of the MTP-FAγ51::AnfD::linker26(HA)::AnfK polypeptide encoded by SN272 Amino acids 1-64 correspond to the MTP-FAγ51-HA sequence including the GG at its C-terminus, amino acids 65-581 correspond to the AnfD sequence (A. vinelandii), amino acids 582-607 correspond to the 26-amino acid linker (Linker26(HA)), and amino acids 608-1068 to AnfK (A. vinelandii).
SEQ ID NO:203. Amino acid sequence of the MTP-CoxIV::AnfD::linker26(HA)-AnfK polypeptide encoded by SN273. Amino acids 1-61 correspond to the MTP-CoxIV sequence including the GG at its C-terminus, amino acids 62-578 correspond to the AnfD sequence (A. vinelandii), amino acids 579-604 correspond to the 26-amino acid linker (Linker26(HA)), and amino acids 605-1065 to AnfK (A. vinelandii).
SEQ ID NO:204. Amino acid sequence of the mFAγ51::AnfD::linker26(HA)::AnfK polypeptide encoded by SN274. Amino acids 1-64 correspond to the mFAγ51 sequence including the alanine substitutions that don't allow for MPP-cleavage and the GG at its C-terminus, amino acids 65-581 correspond to the AnfD sequence (A. vinelandii), amino acids 582-607 correspond to the 26-amino acid linker (Linker26(HA)), and amino acids 608-1068 to AnfK (A. vinelandii).
SEQ ID NO:205. Amino acid sequence of the HISx6::AnfD::linker26(HA)::AnfK polypeptide encoded by SN275, which does not have an MTP sequence and would be cytoplasmically located. Amino acids 1-9 correspond to the HISx6 sequence including the GG at its C-terminus, amino acids 10-526 correspond to the AnfD sequence (A. vinelandii), amino acids 527-552 correspond to the 26-amino acid linker (Linker26(HA)), and amino acids 553-1013 to AnfK (A. vinelandii).
SEQ ID NO:206. Amino acid sequence of the TbHCS polypeptide (Accession No. CP002466).
SEQ ID NO:207. Amino acid sequence of the TpHCS polypeptide (Accession No. CP002028).
SEQ ID NO:208. Amino acid sequence of the ScHCS polypeptide (Accession No. CP036483).
SEQ ID NO:209. Amino acid sequence of the NsHCS polypeptide (Accession No. CP007203).
SEQ ID NO:210. Amino acid sequence of the MaHCS polypeptide (Accession No AE010299) SEQ ID NO:211. Amino acid sequence of the CtHCS polypeptide (Accession No. AE006470).
SEQ ID NO:212. Amino acid sequence of the MiHCS1 polypeptide (Accession No. ADG13125).
SEQ ID NO:213. Amino acid sequence of the MiHCS2 polypeptide (Accession No. ADG13175).
SEQ ID NO:214. Amino acid sequence of the MiHCS3 polypeptide (Accession No. ADG14004).
SEQ ID NO:215. Amino acid sequence of the LjFEN1 polypeptide (Accession No. BAI49592).
SEQ ID NO:216. Amino acid sequence of AnfD from A. vinelandii (Accession No. WP_012703361); 518aa.
SEQ ID NO:217. Amino acid sequence of AnfK from A. vinelandii (Accession No. WP_012703359); 462aa.
SEQ ID NO:218. Amino acid sequence of AnfH from A. vinelandii (Accession No. WP_012703362); 275aa.
SEQ ID NO:219. Amino acid sequence of AnfG from A. vinelandii (Accession No. WP_012703360); 132aa.
SEQ ID NO:220. Peptide sequence.
SEQ ID NO:221. N. benthamiana P72026 amino acid sequence; 606aa.
SEQ ID NO:222. N. benthamiana P20586 amino acid sequence; 470aa.
SEQ ID NO:223. Amino acid sequence of Mycobacterium tuberculosis α-isopropylmalate synthase (MtLeuA); 644aa.
SEQ ID NO:224; Amino acid sequence of the NifH polypeptide from A. vinelandii (AvNifH; Accession No. WP_012698831); 290aa.
SEQ ID NO:225. Peptide sequence, AnfH motif I, where X represents any amino acid.
SEQ ID NO:226. Peptide sequence, AnfH motif II.
SEQ ID NO:227. Peptide sequence, AnfH motif III.
SEQ ID NO:228. Peptide sequence, AnfH motif IV.
SEQ ID NO:229. Peptide sequence, AnfH motif V, where X represents any amino acid.
SEQ ID NO:230. Peptide sequence, AnfH motif VI.
SEQ ID NO:231. Peptide sequence, AnfH motif VII, where X represents any amino acid.
SEQ ID NO:232. Amino acid sequence of the FdxN protein of A. vinelandii; Accession No. WP_012703542; 92aa.
SEQ ID NO:233. Amino acid sequence of the MTP-FAγ51-FdxN-HA fusion polypeptide of SN291; 157aa. Amino acids 1-54 correspond to the MTP-FAγ51sequence with a GG linker, amino acids 55-145 correspond to the FdxN sequence without the N-terminal methionine, and amino acids 146-157 correspond to the HA epitope.
SEQ ID NO:234. Amino acid sequence of the MTP-FAγ51-HA-FdxN fusion polypeptide of SN292; 156aa. Amino acids 1-53 correspond to the MTP-FAγ51sequence with a GG linker, amino acids 54-64 correspond to the HA epitope with a GG linker, and amino acids 65-156 correspond to the FdxN sequence without the N-terminal methionine.
SEQ ID NO:235. Amino acid sequence of the mFAγ51-HA-FdxN fusion polypeptide of SN299; 156aa Amino acids 1-53 correspond to the mFAγ51sequence with a GG linker, amino acids 54-64 correspond to the HA epitope with a GG linker, and amino acids 65-156 correspond to the FdxN sequence without the N-terminal methionine.
SEQ ID NO:236 Amino acid sequence of the HA-FdxN fusion polypeptide of SN300; 104aa. Amino acids 1-12 correspond to the HA epitope with a GG linker, and amino acids 13-104 correspond to the FdxN sequence without the N-terminal methionine.
SEQ ID NO:237. Amino acid sequence of the MTP-FAγ51-HA-NifV fusion polypeptide of SN254; 448aa. Amino acids 1-53 correspond to the MTP-FAγ51sequence with a GG linker, amino acids 54-64 correspond to the HA epitope with a GG linker, and amino acids 65-448 correspond to the NifV sequence from A. vinelandii.
SEQ ID NO:238. Amino acid sequence of the NafY polypeptide from A. vinelandii (AvNafY; Accession No. AGK13761).
SEQ ID NO:239. C-terminal amino acid sequence of a NifK polypeptide.
SEQ ID NO:240. C-terminal amino acid sequence of a NifK polypeptide.
SEQ ID NO:241. C-terminal amino acid sequence of a NifK polypeptide.
SEQ ID NO:242. C-terminal amino acid sequence of a NifK polypeptide.
SEQ ID NO:243. C-terminal amino acid sequence of a NifK polypeptide.
SEQ ID NO:244. C-terminal amino acid sequence of an AnfK polypeptide.
SEQ ID NO:245. C-terminal amino acid sequence of an AnfK polypeptide.
SEQ ID NO:246. C-terminal amino acid sequence of an AnfK polypeptide.
SEQ ID NO:247. C-terminal amino acid sequence of an AnfK polypeptide.
SEQ ID NO:248. C-terminal amino acid sequence of an AnfK polypeptide.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, plant molecular biology, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, or more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Nitrogenase

Nitrogenase is the enzyme in eubacteria and archaeobacteria that catalyses the reduction of the strong, triple bond of nitrogen (N₂) to produce ammonia (NH₃). Nitrogenase is found naturally only in bacteria. It is a complex of two enzymes that can be purified separately, namely dinitrogenase and dinitrogenase reductase. Dinitrogenase, also referred to as component I or the molybdenum-iron (MoFe) protein, is a tetramer of two NifD and two NifK polypeptides (α₂β₂) that also contains two “P-clusters” and two “FeMo-cofactors” (FeMo-co). Each pair of NifD-NifK subunits contains one P-cluster and one FeMo-co. FeMo-co is a metallocluster composed of a MoFe₃—S₃ cluster complexed with a homocitrate molecule, which is coordinated to the molybdenum atom, and bridged to a Fe₄—S₃ cluster by three sulfur ligands. FeMo-co is assembled separately in cells and is then incorporated into apo-MoFe protein. The P-cluster is also a metallocluster and contains 8 Fe atoms and 7 sulfur atoms with a structure similar but different to FeMo-co. The P-clusters are located at the αβ subunit interface of dinitrogenase and are coordinated by cysteinyl residues from both subunits. Dinitrogenase reductase, also referred to as component II or the “Fe protein” is a dimer of NifH polypeptides which also contains a single Fe₄-54 cluster at the subunit interface and two Mg-ATP binding sites, one at each subunit. This enzyme is the obligatory electron donor to the dinitrogenase, where the electrons are transferred from the Fe₄—S₄ cluster to the P-cluster and in turn to the FeMo-co, the site for N₂ reduction.

Although the Mo-containing nitrogenase is the most commonly found nitrogenase in bacteria, there are two homologous nitrogenases that are genetically distinct but have similar cofactor and subunit compositions, namely the vanadium-containing nitrogenase and the Fe-only nitrogenase, encoded by the Vnf (vanadium nitrogen fixation) and Anf (alternative nitrogen fixation) genes, respectively. Some bacteria in nature possess all three types of nitrogenases, other bacteria contain only the Mo- and V-containing enzymes or only the Mo-containing enzyme, for example, Klebsiella pneumoniae.

A variety of nitrogen fixation (Nif) genes are required for the biosynthesis of FeMo-co and maturation of the nitrogenase components to their catalytically active forms. Roles for the NifB, NifE, NifH, NifN, NifQ, NifV and NifX polypeptides in FeMo-co synthesis have been described (Rubio and Ludden, 2008).

Biological N₂ fixation, catalyzed by the prokaryotic enzyme nitrogenase, is an alternative to the use of synthetic N₂ fertilizers. The sensitivity of nitrogenase to oxygen is a major barrier to engineering biological nitrogen fixation into plants, for example, into cereal crops, by direct Nif gene transfer.

The present inventors considered that targeting Nif polypeptides to the mitochondrial matrix (MM) of plant cells might overcome the oxygen sensitivity problem. The MM possesses oxygen consuming enzymes that allow other enzymes that contain an oxygen sensitive Fe—S cluster to function. The mitochondrial Fe—S cluster assembly machinery is similar to diazotrophic equivalents (Balk and Pilon, 2011; Lill and Mühlenhoff, 2008). Therefore some of the requisites for nitrogenase biosynthesis may already be in place in the MM, reducing the number of Nif genes required for reconstitution. There is also a high reducing potential and concentration of ATP (Geigenberger and Fernie, 2014; Mackenzie and McIntosh, 1999), both prerequisites for nitrogenase enzyme catalysis. Additionally the presence of glutamate synthase in mitochondria provides an entry point for any ammonium fixed by nitrogenase to enter plant metabolism. Given these characteristics, and the fact that mitochondria themselves are of α-proteobacterial origin, the present inventors considered that this organelle was well suited as a location for attempting functional reconstitution of nitrogenase.

As a first step towards reconstitution of nitrogenase in plant cell mitochondria, evidence was needed that individual Nif proteins can be correctly targeted to the MM. For this purpose, the inventors chose the model plant Nicotiana benthamiana as an expression platform (Wood et al., 2009) to provide for expression of transgenes either singly or, more importantly, in combinations. As most MM-located proteins are nuclear-encoded, the present inventors relied upon recent advances in understanding the subcellular signalling and transport process (Huang et al., 2009; Murcha et al., 2014), using a previously characterised N-terminal peptide targeting signal (Lee et al., 2012).

The model bacterial diazotroph Klebsiella pneumoniae uses 16 unique proteins for the biosynthesis and catalytic function of nitrogenase. The present inventors re-engineered all 16 Nif proteins from the K. pneumoniae for targeting to the plant MM and assessed their expression and processing in N. benthamiana leaves. All 16 Nif polypeptides were transiently expressed and tested for sequence specific MM processing. The present inventors have established that all of the 16 Nif polypeptides can be individually expressed as MTP:Nif fusion polypeptides in plant leaf cells. Furthermore, the present inventors provide evidence that these proteins can be targeted to the mitochondrial matrix (MM), a subcellular location potentially accommodating for nitrogenase function and can be cleaved by mitochondrial processing protease (MPP). This represents important progress towards the aim of engineering endogenous nitrogen fixation in plants.

### Mitochondrial Protein Import in Plants

Almost all mitochondrial proteins are nuclear encoded and translated in the cytosol, therefore requiring their translocation into the mitochondria. Signal sequences within the polypeptides direct their import to four different intra-mitochondrial locations:

the outer membrane (OM), the intermembrane space (IS), the inner membrane (IM), or the matrix (MM). These signal sequences are distinguished by their biochemical properties and guide trafficking via at least four distinct import pathways which direct the polypeptides to one or more of the four locations (Chacinska et al., 2009). These four pathways are: (1) the general import pathway, also referred to as the “classical” pre-sequence pathway, which directs polypeptides to the MM, the IS or the IM; (2) the carrier import pathway, used for transport to the IM, (3) the mitochondrial intermembrane space (MIA) assembly pathway, and (4) the sorting and assembly machinery (SAM) pathway used for transport of polypeptides to the OM. The general import pathway imports polypeptides having a cleavable pre-sequence, also known as a signal sequence. These polypeptides may also have a hydrophobic sorting signal (HSS). The carrier import pathway imports polypeptides with internal pre-sequence like signals and a hydrophobic region. The MIA pathway imports polypeptides with twin cysteine residues. The SAM pathway imports polypeptides that contain a 13 signal and a putative TOM20 signal. All of these pathways make use of a translocase of the outer membrane (TOM) and the first and second pathways also use a TIM23 translocase of the intermembrane complex. Only the first pathway uses matrix processing peptidase (matrix processing protease, MPP).

A common characteristic of all mitochondrial targeted polypeptides is the presence of at least one domain within the polypeptide that guides transport to the correct location. The best studied of these is the “classic” N-terminal pre-sequence domain that is cleaved in the matrix by MPP (Murcha et al., 2004). It has been estimated that about 70% of plant and animal mitochondrial proteins have a cleavable pre-sequence but both internal and C-terminal signal sequences have also been found (reviewed in Pfanner and Geissler (2001), Schleiff and Soll (2000)). In Arabidopsis, these pre-sequences range in length from 11 to 109 amino acid residues with an average length of 50 amino acid residues. Although there is no consensus sequence that fully defines a pre-sequence for the first pathway, they tend to contain a high proportion of hydrophobic and positively charged amino acids. A further characteristic is their ability to form an amphiphilic α-helix, usually starting within the first 10 amino acid residues (Roise et al., 1986). These domains are rich in hydrophobic (Ala, Leu, Phe, Val), hydroxylated (Ser, Thr) and positively charged (Arg, Lys) amino acid residues, and deficient in acidic amino acids. Over a large number of mitochondrial proteins, serine (16-17%) and alanine (12-13%) are greatly over-represented in mitochondrial signal peptides, and arginine is abundant (12%). The MPP cleavage point is defined for most pre-sequences by the presence of a conserved arginine residue, usually at position P2 (-2 aa from the scissile bond), or P3 in most other cases (Huang et al., 2009).

Mitochondrial pre-sequences interact with the Tom20 receptor through hydrophobic residues. Studies have shown that the hydrophobic surface of the α-helix facilitates recognition of the peptide by the TOM20 component of the TOM import complex, whereas the positive charges are recognised by the TOM22 subunit (Abe et al., 2000). Finally, most pre-sequences guide transport of the polypeptide in association with Hsp70, and accordingly nearly all plant pre-sequences contain at least one binding motif for Hsp70 molecular chaperone (Zhang and Glaser, 2002). The chaperone Hsp70 is involved in protein folding, prevents protein aggregations, and functions as a molecular motor, pulling the precursor across the mitochondrial membranes. The electrical membrane potential (Δψ) (˜100 mV, negative inside) across the inner membrane also drives translocation of the positively charged pre-sequence via an electrophoretic effect.

The majority of proteins with cleavable pre-sequences are destined for the mitochondrial matrix via the general import pathway, which utilises the transporter of the outer membrane (TOM) complex and the transporter of the inner membrane 23 complex (TIM23). However some proteins with cleavable pre-sequences can assemble in the inner membrane (Murcha et al., 2004) or the inter membrane space, if they also contain a hydrophobic sorting signal (HSS) (Glick et al., 1992). There are very few examples of matrix localised proteins that do not have their pre-sequences cleaved. In Arabidopsis, only Glutamate dehydrogenase has been found in the matrix with an unprocessed full length pre-sequence (Huang et al., 2009).

For proteins that are not matrix targeted, a variety of internal non-cleavable localisation signals are employed. These are typically associated with a specific trafficking pathway, and are additionally tailored for the particular class of protein. In plants, no studies thus far have determined what precisely constitutes an internal signal sequence for intermembrane space proteins. However, it appears a motif with twin cysteine residues is associated with transport via the mitochondrial intermembrane space assembly pathway (MIA) (Carrie et al., 2010; Darshi et al., 2012). Finally, non-cleavable internal sequences are also utilised by proteins destined for the inner membrane via the carrier pathway, which utilises the TOM and TIM22 apparatus to insert proteins with multiple transmembrane regions (Kerscher et al., 1997; Sirrenberg et al., 1996). These sequences typically contain a hydrophobic region followed by a pre-sequence like internal sequence, and are thus similar to N-terminal pre-sequences, but distinguished by their internal location within their cognate protein.

In photosynthetic organisms, nuclear encoded mitochondrial proteins have a requirement for differentiation between chloroplast and mitochondrial trafficking, despite many similarities between these two organelles and their proteomes. The α-helix that occurs mostly in mitochondria pre-sequences is usually absent in chloroplast pre-sequences (Zhang and Glaser, 2002), which tend to be more unstructured and show high β sheet domain structure (Bruce, 2001).

In plants, the MPP is anchored to the inner membrane bound Cytbci complex, although the active MPP site is located facing the matrix, and the functions of the two proteins are independent (Glaser and Dessi, 1999).

### Mitochondrial Targeting Peptide

As used herein, the term “mitochondrial targeting peptide” or “MTP” means an amino acid sequence, comprising at least 10 amino acids and preferably between 10 and about 80 amino acid residues in length that directs a target protein to a mitochondrion and which can be used heterologously in an MTP-target protein translational fusion to direct a selected target protein such as a Nif polypeptide, Gus, GFP etc to a mitochondrion.

The MTP typically comprises at its N-terminus a translation initiator methionine of the polypeptide from which it is derived. The MTP is translationally fused to a Nif polypeptide or “target protein” by a peptide bond to the Met residue that corresponds to the initiator Met of the target protein, or that Met residue may be omitted and the peptide bond is directly fused to the amino acid residue that in the wild-type is the second amino acid of the target protein. The MTP is typically rich in basic and hydroxylated amino acids and usually lacks acidic amino acids or extended hydrophobic stretches. The MTP may form amphiphilic helices.

While not wanting to be limited by theory, the MTP typically comprises an uptake-targeting sequence that binds to receptors on the outer membrane of the mitochondrion. Upon binding to the outer membrane, the fusion polypeptide preferably undergoes membrane translocation to transport channel proteins, and passages through the double membrane of the mitochondrion to the mitochondrial matrix (MM). The uptake-targeting sequence is then typically cleaved and the mature fusion protein folded. The MTP may comprise additional signals that subsequently target the protein to different regions of the mitochondria, such as the mitochondrial matrix (MM). In an embodiment, the uptake-targeting sequence is a matrix targeting sequence.

The MTP may be cleavable or non-cleavable when translationally fused to the Nif polypeptide. Thus, in an embodiment, the MTP-Nif fusion polypeptide is at least partially cleaved. In this regard, the phrase “at least partially cleaved” refers to a detectable amount of cleavage of a MTP-Nif fusion polypeptide when expressed in a plant cell. In an embodiment, at least 50% of the MTP-Nif fusion polypeptide that is produced in the cell is cleaved within the MTP sequence, preferably at least 75% is cleaved, more preferably at least 90% is cleaved. In an alternative embodiment, less than 50% of the MTP-Nif fusion polypeptide is cleaved in the cell, for example, the MTP is not cleaved. In an embodiment, the MTP does not comprise a cleavage site for MPP. The MTP may comprise a cleavage site. Upon cleavage, the N-terminal part of the resultant processed product (i.e., the mature NP) may comprise one or more C-terminal amino acids of the MTP, also referred to herein as a “scar sequence” or “scar peptide”, or it may not comprise any C-terminal amino acids of the MTP. When present, the scar sequence is preferable 1 to 45 amino acids in length, more preferably 1 to 20 amino acids, even more preferably 1 to 12 amino acids. Alternatively, the cleavage site may be located within the fusion polypeptide such that the entire MTP sequence is cleaved off, for example, the linker may comprise the cleavage sequence.

Native mitochondrial targeting peptides are localized at the N-terminus of the precursor proteins and a N-terminal part are typically cleaved off during or after import into mitochondria. Cleavage is typically catalysed by the general matrix processing protease (MPP), which, in plants, is integrated into the bc₁ complex of the respiratory chain. This protease recognizes the cleavage sites of nearly 1000 precursor proteins that have a wide range of amino acid sequences which show little conservation. In an embodiment, the MTP comprises a protease cleavage site for MPP. In a further embodiment, the processed product is produced by cleavage of the fusion protein within, or immediately after, the MTP by MPP. In this context, the phrase “immediately after” means that following cleavage by MPP, there are no amino acids remaining from the MTP fused to the Nif polypeptide. Thus, where the fusion polypeptide is cleaved “immediately after” the MTP, the MPP cleavage site is immediately after the C-terminal amino acid of the MTP.

The terms “cleaved product” or “cleavage product”, as used herein in the context of a MTP fusion polypeptide, refer to a polypeptide resulting from protease cleavage either within or immediately after the MTP amino acid sequence. In this regard, the cleaved product of the MTP fusion polypeptide is obtainable by cleavage by MPP. The cleaved product may retain one or more amino acids from the MTP after cleaveage (i.e., a scar peptide), or it may not have any amino acids remaining from the MTP after cleavage. In an embodiment, a cleaved product of a Nif fusion polypeptide of the invention comprises at least 95% or all of the amino acids present in the Nif polypeptide sequence.

In an embodiment, the MTP is not cleaved. The present inventors have demonstrated that incorporation of the MTP did not always lead to complete processing of Nif proteins. In some instances (NifX-FLAG, NifD-HAₒₚₜ₁ and NifDK-HA), both processed and unprocessed Nif proteins were observed. Considering there is no general consensus sequence for MTPs, and internal protein sequences can influence mitochondrial targeting (Becker et al., 2012), it is perhaps not surprising that the present inventors found differences in processing efficiency amongst the Nif proteins.

Suitable MTPs that can be used in the context of the present invention include, without limitation, peptides having the general structure as defined by von Heijne (1986) or by Roise and Schatz (1988). Non limiting examples of MTPs are the mitochondrial targeting peptides defined in Table I of von Heijne (1986) or disclosed herein.

In an embodiment, the MTP is an F1-ATPase γ-subunit (MTP-FAγ). An example of a suitable FAγ MTP is that from A. thaliana (Lee et al., 2012). In an embodiment, the MTP-FAγ is 77 amino acids in length, the cleavage of which by an MMP leaves 35 MTP residues at the N-terminal end of the fusion polypeptide. In a preferred embodiment, the MTP-FAγ is less than 77 amino acids in length. For example, the MTP-FAγ may be about 51 amino acids in length, the cleavage of which by an MMP leaves 9 MTP residues at the N-terminal end of the fusion polypeptide.

The skilled person will appreciate that software exists for predicting mitochondrial proteins and their targeting sequence, for example, MitoProtII, PSORT, TargetP and NNPSL.

MitoProtII is a program that predicts mitochondrial localization of a sequence based on several physiochemical parameters (e.g., amino acid composition in the N-terminal part, or the highest total hydrophobicity for a 17 residues window). P SORT is a program that predicts subcellular locations based on various sequence-derived features such as the presence of sequence motifs and amino acid compositions. TargetP predicts the subcellular location of eukaryotic proteins based on the predicted presence of any of the N-terminal presequences: chloroplast transit peptide, mitochondrial targeting peptide or secretory pathway signal peptide. TargetP requires the N-terminal sequence as an input into two layers of artificial neural networks (ANN), utilizing the earlier binary predictors, SignalP and ChloroP. For the sequences predicted to contain an N-terminal presequence a potential cleavage site can also be predicted. NNPSL is another ANN-based method using the amino acid composition to assign one of four subcellular localization (cytosolic, extracellular, nuclear and mitochondrial) to a query sequence.

The skilled person would be readily able to determine if the chosen MTP targeted the fusion polypeptide to the mitochondrial matrix based on routine methods and methods disclosed herein. The present inventors chose a targeting peptide previously demonstrated as capable of transporting GFP in Arabidopsis protoplasts (Lee et al., 2012), and which is relatively long, to assist detection of processed protein. As shown in the Examples herein, the chosen MTP targeted all of the selected nitrogenase proteins to the MM. This conclusion is based on several lines of evidence. Firstly, the sizes observed for N. benthamiana expressed Nif polypeptides were consistent with the expected size resulting from MM peptidase processing. This was also reflected by the difference in size observed between bacterial (full length unprocessed), and plant mitochondrial expressed Nifs of small sizes (NifF and NifZ). Additionally, mutation of the MTP, rendering it incapable of being processed by the mitochondrial import machinery, produced a larger band for both NifD and GFP fusions, consistent with the difference in size between processed and unprocessed protein. Finally, mass spectrometry for an exemplary fusion polypeptide determined that MTP-NifH was cleaved between residues 42-43 of the MTP as predicted for specific processing in the matrix.

It may be useful in some embodiments of this invention to use multiple tandem copies of a chosen MTP. The coding sequence for a duplicated or multiplied targeting peptide may be obtained through genetic engineering from an existing MTP. The amount of MTP can be measured by cellular fractionation, followed by, for example, quantitative immunoblot analysis. Thus, in the present invention, the term “mitochondrial targeting peptide” or “MTP” encompasses one or more copies of one amino acid peptide that directs a target Nif protein to the mitochondria. In a preferred embodiment, the MTP comprises two copies of a chosen MTP. In another embodiment, the MTP comprises three copies of a chosen MTP. In another embodiment, the MTP comprises four copies or more of a chosen MTP.

The skilled person will appreciate that the MTP sequence is not limited to native MTP sequences but may comprise amino acid substitutions, deletions and/or insertions, relative to a naturally-occurring MTP, provided that the sequence variant still functions for mitochondrial targeting.

The skilled person will understand that the MTP may be flanked by amino acids at its N- or C-terminal ends as a result of the cloning strategy and may function as a linker. These additional amino acids may be considered to form part of the MTP.

The skilled person will also understand that the MTP may be N- or C-terminally fused to an oligopeptide linker and/or tag such as an epitope tag. In a preferred embodiment, one or more or all of the Nif fusion polypeptides of the invention produced in a plant cell lack added epitope tags relative to a corresponding wild-type Nif polypeptide.

### Mitochondrial Targeting Peptide (MTP)-Nif Fusion Polypeptides

The present invention relates to mitochondrial targeting peptide (MTP)-Nif fusion polypeptides and their cleaved polypeptide products. When an MTP-Nif fusion polypeptide of the invention is expressed in a plant cell, either the MTP-Nif fusion polypeptide and/or the cleaved polypeptide product is targeted to the mitochondrial matrix (MM). Preferably, the fusion polypeptides confer nitrogenase reductase and/or nitrogenase activity to the plant cell, or an activity which is the same as that conferred by a corresponding wild-type Nif polypeptide in bacteria.

As used herein, the term “fusion polypeptide” means a polypeptide which comprises two or more polypeptide domains which are covalently joined by a peptide bond. Typically, the fusion polypeptide is encoded as a single polypeptide chain by a chimeric polynucleotide of the invention. In an embodiment, fusion polypeptides of the invention comprise a mitochondrial targeting peptide (MTP) and a Nif polypeptide (NP). In this embodiment, the C-terminal end of the MTP is translationally fused to the N-terminal end of the NP. In an alternative embodiment, fusion polypeptides of the invention comprise a C-terminal part of an MTP and a NP, where the C-terminal part results from cleavage of the MTP by MPP. Such a C-terminal part of an MTP is referred to herein as a “scar” sequence. In this embodiment, the C-terminal amino acid of the C-terminal part of the MTP is translationally fused to the N-terminal amino acid of the NP. In these embodiments, the fusion polypeptide may comprise one or more additional amino acids between the MTP and the NP, such as a GlyGly sequence, and/or an added methionine as a translation start amino acid. In an embodiment, the fusion polypeptide comprises two Nif polypeptides, preferably a NifD polypeptide translationally fused via a linker sequence to a NifK polypeptide or a NifE polypeptide translationally fused via a linker sequence to a NifN polypeptide. Both of these fused polypeptides may be present. In these embodiments, it is preferred that the second Nif polypeptide in the fusion polypeptide has its wild-type C-terminus, i.e., lacking any C-terminal extension.

As used herein, the term “translationally fused at the N-terminal end” means that the C-terminal end of the MTP polypeptide or linker polypeptide is covalently joined by a peptide bond to the N-terminal end of a NP, thereby being a fusion polypeptide. In an embodiment, the NP does not comprise its native translation start methionine (Met) residue or its two N-terminal Met residues relative to a corresponding wild-type NP. In an alternative embodiment, the NP comprises the translation start Met or one or both of the two N-terminal Met residues of the wild-type NP polypeptide such as, for example, for NifD.

Such polypeptides are typically produced by expression of a chimeric protein coding region where the translational reading frame of the nucleotides encoding the MTP are joined in-frame with the reading frame of the nucleotides encoding the NP. The skilled person will appreciate that the C-terminal amino acid of the MTP can be translationally fused to the N-terminal amino acid of the NP without a linker or via a linker of one or more amino acid residues, for example of 1-5 amino acid residues. Such a linker can also be considered to be part of the MTP. Expression of the protein coding region may be followed by cleavage of the MTP in the MM of a plant cell, and such cleavage (if it occurs) is included in the concept of production of the fusion polypeptide of the invention.

The fusion polypeptide or the processed Nif polypeptide preferably has functional Nif activity. In a preferred embodiment, the activity is similar to that of the corresponding wild-type Nif polypeptide. The functional activity of the fusion polypeptide or the processed Nif polypeptide may be determined in bacterial and biochemical complementation assays. In a preferred embodiment, the fusion polypeptide or the processed Nif polypeptide has between about 70-100% of the activity of the wild-type Nif activity. Nif polypeptides which do not have Nif function still have utility, for example, as research tools to test for expression levels from genetic constructs or for association with other Nif polypeptides.

The fusion polypeptide may comprise more than one MTP and/or more than one NP, for example, the fusion polypeptide may comprise a MTP, a NifD polypeptide and a NifK polypeptide. The fusion polypeptide may also comprise an oligopeptide linker, for example, linking two NPs. Preferably, the linker is of sufficient length to allow the two or more functional domains, for example, two NPs such as NifD and NifK or NifE and NifN, to associate in a functional configuration in a plant cell. In a preferred embodiment, the NifD polypeptide is an AnfD polypeptide and the NifK polypeptide is an AnfK polypeptide. Such a linker may be between 8 and 50 amino acid residues in length, preferably about 25-35 amino acids in length, more preferably about 30 amino acid residues in length or about 26 amino acid residues in length for an AnfD-linker-AnfK fusion polypeptide. A fusion polypeptide may be obtained by conventional means, e.g., by means of gene expression of the polynucleotide sequence encoding for said fusion polypeptide in a suitable cell.

As used herein, a “substantially purified polypeptide” means a polypeptide which is substantially free from components (e.g., lipids, nucleic acids, carbohydrates) that normally associate with the polypeptide, for example, in a cell. Preferably, the substantially purified polypeptide is at least 90% free from said components.

Plant cells, transgenic plants and parts thereof of the invention comprise a polynucleotide encoding a polypeptide of the invention. Polypeptides of the invention are not naturally occurring in plant cells, in particular not in the mitochondria of plant cells, and therefore the polynucleotide encoding the polypeptide may be referred to herein as an exogenous polynucleotide since it is not naturally occurring in a plant cell but has been introduced into the plant cell or a progenitor cell. The cells, plants and plant parts of the invention which produce a polypeptide of the invention can therefore be said to produce a recombinant polypeptide. The term “recombinant” in the context of a polypeptide refers to the polypeptide encoded by an exogenous polynucleotide when produced by a cell, which polynucleotide has been introduced into the cell or a progenitor cell by recombinant DNA or RNA techniques such as, for example, transformation. Typically, the plant cell, plant or plant part comprises a non-endogenous gene that causes an amount of the polypeptide to be produced, at least at some time in the life-cycle of the plant cell or plant. Preferably the exogenous polynucleotide is integrated into the nuclear genome of the plant cell and/or is transcribed in the nucleus of the cell.

In an embodiment, a polypeptide of the invention is not a naturally occurring polypeptide. In an alternative embodiment, the polypeptide of the invention is naturally occurring but is present in a plant cell, preferably in a mitochondrion of a plant cell, in which it does not naturally occur.

In an embodiment, a polypeptide of the invention (e.g., a MTP fusion polypeptide or cleaved product thereof) is at least partially soluble in mitochondria of a plant cell. In this context, the phrase “at least partially soluble” means that the polypeptide is detectable in the soluble fraction of a homogenised sample comprising mitochondria of a plant cell. Suitable methods for detecting solubility of polypeptides are known in the art and include those that are described in Example 1. In an embodiment, at least 5%, at least 10%, at least 15%, at least 20%, at least 25%, at least 30%, at least 40%, at least 50%, at least 60%, at least 70%, at least 80%, or at least 90% of the polypeptpide present in the cell is soluble.

### Nif Polypeptides

As used herein, the terms “Nif polypeptide” and “Nif protein” are used interchangeably and mean a polypeptide which is related in amino acid sequence to naturally occurring polypeptides involved in nitrogenase activity, where the Nif polypeptide of the invention is selected from the group consisting of a NifD polypeptide, a NifH polypeptide, a NifK polypeptide, a NifB polypeptide, a NifE polypeptide, a NifN polypeptide, a NifF polypeptide, a NifJ polypeptide, a NifM polypeptide, a NifQ polypeptide, a NifS polypeptide, a NifU polypeptide, a NifV polypeptide, a NifW polypeptide, a NifX polypeptide, a NifY polypeptide and a NifZ polypeptide, each of which as defined herein. Nif polypeptides of the invention include “Nif fusion polypeptides” which, as used herein, means a polypeptide homolog of a naturally occurring Nif polypeptide that has additional amino acid residues joined to the N-terminus or C-terminus, or both, relative to a corresponding naturally occurring Nif polypeptide. As mentioned above, the Nif fusion polypeptide may be lacking the translation initiation Met or the two N-terminal Met residues relative to a corresponding wild-type Nif polypeptide. The amino acid residues of a Nif fusion polypeptide that correspond to the naturally occurring Nif polypeptide, i.e., without the additional amino acid residues joined to the N-terminus or C-terminus or both, are also referred to herein as a Nif polypeptide, abbreviated in this case to “NP”, or as a NifD polypeptide (“ND”) etc. In a preferred embodiment, the “additional amino acid residues joined to the N-terminus or C-terminus or both” comprise a mitochondrial targeting peptide (MTP) or a processed MTP joined to the N-terminus of the NP, or an epitope sequence (“tag”) which is N-terminal or C-terminal to the NP or both, or both an MTP or processed MTP and an epitope sequence.

Naturally occurring Nif polypeptides occur only in some bacteria including the nitrogen-fixing bacteria, including free living nitrogen fixing bacteria, associative nitrogen fixing bacteria and symbiotic nitrogen fixing bacteria. Free living nitrogen fixing bacteria are capable of fixing significant levels of nitrogen without the direct interaction with other organisms. Without limitation, said free living nitrogen fixing bacteria include the members of the genera Azotobacter, Beijerinckia, Klebsiella, Cyanobacteria (classified as aerobic organisms) and the members of the genera Clostridium, Desulfovibrio and the named purple sulphur bacteria, purple non-sulphur bacteria and green sulphur bacteria. Associative nitrogen fixing bacteria are those prokaryotic organisms that are able to form close associations with several members of the Poaceae (grasses). These bacteria fix appreciable amounts of nitrogen within the rhizosphere of the host plants. Members of the genera Azospirillum are representative of associative nitrogen fixing bacteria. Symbiotic nitrogen fixation bacteria are those bacteria which fix nitrogen symbiotically by partnering with a host plant. The plant provides sugars from photosynthesis that are utilized by the nitrogen fixing bacteria for the energy it needs for nitrogen fixation. Members of the genera Rhizobia are representative of associative nitrogen fixing bacteria.

The Nif polypeptide or Nif fusion polypeptide of the invention is selected from the group consisting of NifH, NifD, NifK, NifB, NifE, NifN, NifF, NifJ, NifM, NifQ, NifS, NifU, NifV, Nitta, NifX, NifY and NifZ polypeptides. Function of these polypeptides has been reviewed recently by Buren et al. (2020).

Other polypeptides of the invention are considered to be VnfG and AnfG involved in the V-nitrogenase and Fe-nitrogenase, respectively, nitogenase associated factors (Naf polypeptides) such as, for example, NafY, and ferredoxin polypeptides such as FdxN polypeptides. These polypeptides are preferably encoded and expressed as MTP-fusion polypeptides for mitochondrial targeting.

A polypeptide or class of polypeptides may be defined by the extent of identity (% identity) of its amino acid sequence to a reference amino acid sequence and/or by the presence of certain amino acid motifs or protein family domains, or by having a greater % identity to one reference amino acid sequence than to another. A polypeptide or class of polypeptides may also be defined by having the same biological activity as a naturally occurring Nif polypeptide, in addition to the extent of identity in sequence.

The % identity of a polypeptide is determined by GAP (Needleman and Wunsch, 1970) analysis (GCG program) with a gap creation penalty=5, and a gap extension penalty=0.3, or by Blastp version 2.5 or updated versions thereof (Altschul et al., 1997), where in each case the analysis aligns two sequences including a reference sequence over the entire length of the reference sequence. As used herein, reference sequences include those provided for naturally occurring Nif polypeptides from K. pneumoniae (renamed as K. oxytoca), SEQ ID NOs:1-17.

In the following definitions, the extent of identity of an amino acid sequence to a reference sequence provided as a SEQ ID NO is determined by Blastp, version 2.5 or updated versions thereof (Altschul et al,. 1997), using the default parameters except for the maximum number of target sequences which is set at 10,000, and is determined along the full length of the reference amino acid sequence.

A NifH polypeptide in naturally occurring bacteria is a structural component of nitrogenase complex and is often termed the iron (Fe) protein. It forms a homodimer, with a Fe₄S₄ cluster bound between the subunits and two ATP-binding domains. NifH is the obligate electron donor to the nitrogenase protein (NifD/NifK heterotetramer) and therefore functions as the nitrogenase reductase (EC 1.18.6.1). NifH of the molybdenum type is also involved in FeMo-co biosynthesis and apo-MoFe protein maturation (Jasniewski et al., 2018). As reviewed therein, NifH has three primary recognised functions: (i) involvement in the insertion of Mo and homocitrate in the synthesis of FeMo-co, also involving the NifE-NifN complex, (ii) a reductase function in the formation of P-cluster on NifD-NifK from what is termed P* cluster, which may also involve a small chaperone-like polypeptide NifZ, and (iii) as electron donor to the nitrogenase protein.

As used herein, a “NifH polypeptide” means a polypeptide comprising amino acids whose sequence is at least 41% identical to the amino acid sequence provided as SEQ ID NO:1 and which comprises one or more of the domains TIGR01287, PRK13236, PRK13233 and cd02040. The TIGR01287 domain is present in each of molybdenum-iron nitrogenase reductase (NifH), vanadium-iron nitrogenase reductase (VnfH), and iron-iron nitrogenase reductase (AnfH) but excludes the homologous protein from the light-independent protochlorophyllide reductase. As used herein, NifH polypeptides therefore include the subclass of iron-binding polypeptides which comprise amino acids whose sequence is at least 41% identical to SEQ ID NO:1, the VnfH iron-binding polypeptides and the AnfH iron-binding polypeptides. A naturally occurring NifH polypeptide typically has a length of between 260 and 300 amino acids and the natural monomer has a molecular weight of about 30 kDa. A great number of NifH polypeptides have been identified and numerous sequences are available in publically available databases. For example, NifH polypeptides have been reported from Klebsiella michiganensis (Accession No. WP_049123239.1, 99% identical to SEQ ID NO:1), Brenneria goodwinii (WP_048638817.1, 93% identical), Sideroxydans lithotrophicus (WP_013029017.1, 84% identical), Denitrovibrio acetiphilus (WP_013010353.1, 80% identical), Desulfovibrio africanus (WP_014258951.1, 72% identical), Chlorobium phaeobacteroides (WP_011744626.1, 69% identical), Methanosaeta concilii (WP_013718497.1, 64% identical), Rhodobacter (WP_009565928.1, 61% identical), Methanocaldococcus infernus (WP_013099472.1, 42% identical) and Desulfosporosinus youngiae (WP_007781874.1, 41% identical). NifH polypeptides have been described and reviewed in Thiel et al. (1997), Pratte et al. (2006), Boison et al. (2006) and Staples et al. (2007).

As used herein, a functional NifH polypeptide is a NifH polypeptide which is capable of forming a functional nitrogenase protein complex together with the other required subunits, for example, NifD and NifK, and the FeMo-, FeV- or FeFe-cofactor.

As used herein, an “AnfH polypeptide” is a NifH polypeptide which is a member of the nitrogenase conserved superfamily c125403 (TIGR01287) containing the PRK13233 conserved domain and having at least 69% amino acid sequence identity to the Azotobacter vinelandii AnfH polypeptide (SEQ ID NO:218; Accession No. WP_012703362) when measured along the full-length of SEQ ID NO:218. This amino acid sequence is used herein as the reference sequence for AnfH. TIGR01287AnfH represents the all-iron variant of the nitrogenase component II, also known as nitrogenase reductase. As used herein, the AnfH polypeptides are a subset of the NifH polypeptides. AnfH polypeptides do not include the molybdenum type NifH polypeptides and the vanadium type NifH polypeptides (VnfH). The amino acid sequences of AnfH polypeptides in sequence databases were usually annotated as an AnfH polypeptide. As of January 2020, there were 314 specific amino acid sequences in the NCBI protein database in the AnfH set, all of which had amino acid residues specific to AnfH and which were distinct from the molybdenum-type NifH and VntH, which subsets looked more alike but still distinct. Examples of naturally occurring AnfH polypeptides include AnfH polypeptides from Rhodocyclus tenuis (Accession No. WP_153472986; 92.36% identical), Dickeya paradisiaca (Accession No. WP_015854293; 88.36% identical), Thermodesulfitimonas autotrophica (Accession No. WP_123927773; 78.91% identical), Clostridium kluyveri (Accession No. WP_073538802; 76.36% identical) and Methanophagales archaeon (Accession No. RCV64832; 69.37% identical), each with reference to SEQ ID NO:218.

As described in Example 23 herein, 16 amino acids were identified at defined positions in AnfH sequences that were conserved and characteristic of AnfH polypeptides relative to the molybdenum-type NifH sequence of AvNifH. These can be used to distinguish AnfH polypeptides from other NifH sequences which do not have all 16 amino acids in common. AvNifH, KoNifH (SEQ ID NO:1) and other molybdenum type NifH sequences had motifs III and IV but did not have motifs I, II, V-VII, and therefore these motifs (SEQ ID NOs:225-231) could also be used to distinguish the AnfH subset from other NifH polypeptides.

Analogous to other functional NifH polypeptides, functional AnfH polypeptides are capable of functioning as a nitrogenase reductase, being the obligate electron donor to FeFe complex. Analogous to the molybdenum-type NifH, AnfH is potentially involved in FeFe-co biosynthesis and maturation of the apo-FeFe complex (AnfD-AnfK-AnfG).

As used herein, a “NifD polypeptide” means a polypeptide comprising amino acids whose sequence is at least 33% identical to the amino acid sequence provided as SEQ ID NO:2 and which comprises (i) one or both of the domains TIGR01282 and COG2710, both of which are found in the iron-molybdenum binding polypeptides including the polypeptide having the amino acid sequence shown in SEQ ID NO:2, or (ii) the iron-vanadium binding domain TIGR01860 in which case the NifD polypeptide is in the subclass of VnfD polypeptides, or (iii) the iron-iron binding domain TIGR1861 in which case the NifD polypeptide is in the subclass of AnfD polypeptides. The NifD polypeptide may be part of a fusion polypeptide, for example, fused to a MTP and/or NifK, or alternatively may not comprise any N- or C-terminal extensions. In a preferred embodiment, the NifD polypeptide when associated with a NifK polypeptide, binds FeMo-cofactor.

As used herein, NifD polypeptides include the subclass of iron-molybdenum (FeMo-co) binding polypeptides comprising amino acids whose sequence is at least 33% identical to SEQ ID NO:2, the VnfD iron-vanadium polypeptides and the AnfD polypeptides. A naturally occurring NifD polypeptide typically has a length of between 470 and 540 amino acids. A great number of NifD polypeptides have been identified and numerous sequences are available in publically available databases. For example, NifD polypeptides have been reported from Raoultella ornithinolytica (Accession No. WP_044347161.1, 96% identical to SEQ ID NO:2), Kluyvera intermedia (WP_047370273.1, 93% identical), Dickeya dadantii (WP_038902190.1, 89% identical), Tolumonas sp. BRL6-1 (WP_024872642.1, 81% identical), Magnetospirillum gryphiswaldense (WP_024078601.1, 68% identical), Thermoanaerobacterium thermosaccharolyticum (WP_013298320.1, 42% identical), Methanothermobacter thermautotrophicus (WP_010877172.1, 38% identical), Desullovibrio africanus (WP_014258953.1, 37% identical), Desulfotomaculum sp. LMa1 (WP_066665786.1, 37% identical), Desulfomicrobium baculatum (WP_015773055.1, 36% identical), the VnfD polypeptide of Fischerella muscicola (WP_016867598.1, 34% identical) and the AnfD polypeptide from Opitutaceae bacterium TAV5 (WP_009512873.1, 33% identical). NifD polypeptides have been described and reviewed in Lawson and Smith (2002), Kim and Rees (1994), Eady (1996), Robson et al. (1989), Dilworth et al. (1988), Dilworth et al. (1993), Miller and Eady (1988), Chiu et al. (2001), Mayer et al. (1999), and Tezcan et al. (2005).

NifD polypeptides of the iron-molybdenum subclass are a key subunit of nitrogenase complexes, being the a subunit of the α₂β₂ MoFe protein complex at the core of nitrogenase, and the site of substrate reduction with the FeMo cofactor. As used herein, a functional NifD polypeptide is a NifD polypeptide which is capable of forming a functional nitrogenase protein complex together with the other required subunits, for example, NifH and NifK, and the FeMo or other cofactor.

As used herein a “a NifD polypeptide (ND) which is resistant to protease cleavage” is resistant to cleavage at a defined site or within a defined region, for example within an amino acid sequence corresponding to amino acids 97-100 of SEQ ID NO:18, when the ND is introduced into plant mitochondria by use of an MTP. As used herein “resistant to protease cleavage” means yielding <10% cleavage when the NifD polypeptide is introduced into plant mitochondria by use of an MTP. In preferred embodiments, less than 5% of the NifD polypeptide is cleaved at the site or within the region, more preferably essentially not cleaved, or cleavage is not detected. The NifD polypeptide may be “relatively resistant to cleavage” compared to a NifD polypeptide comprising the amino acid sequence provided as SEQ ID NO:18, being cleaved at least 5-fold less often, preferably at least 10-fold less often, as a NifD polypeptide comprising the amino acid sequence provided as SEQ ID NO:18.

As used herein, an “amino acid sequence other than RRNY (SEQ ID NO:101) at positions corresponding to amino acids 97-100 of SEQ ID NO:18” refers to a sequence which comprises four residues at positions corresponding to amino acids 97-100 of SEQ ID NO:18 and which is not RRNY.

As used herein, an “AnfD polypeptide” is a NifD polypeptide which is specifically a member of the oxidoreductase nitrogenase conserved superfamily c130843, containing the TIGR01861 conserved domain, and having at least 71% amino acid sequence identity to the Azotobacter vinelandii AnfD polypeptide (SEQ ID NO:216; Accession No. WP_012703361) when measured along the full-length of SEQ ID NO:216. This amino acid sequence is used herein as the reference sequence for AnfD TIGR01861:AnfD represents the all-iron variant of the nitrogenase component I α-chain. As used herein, an AnfD polypeptide is therefore a subset of the NifD polypeptides. AnfD polypeptides do not include the molybdenum type NifD polypeptides and the vanadium type NifD polypeptides (VnfD) and also do not include protochlorophyllide or chlorophyllide reductase polypeptides (Boyd and Peters, 2013). The amino acid sequences of AnfD polypeptides in the protein sequence database are usually annotated as an AnfD polypeptide. As of January 2020, there were 156 specific amino acid sequences in the NCBI protein database in the AnfD set. Examples of naturally occurring AnfD polypeptides include AnfD polypeptides from Desulfovibrio sp. DV (Accession No. WP_075356167; 87.47% identical), Paenibacillus sp. FSL H7-0357 (Accession No. WP_038590013; 85.52% identical), Rhodobacter capsulatus (Accession No. WP 023922817; 80.31% identical), Methanosarcina acetivorans C2A (Accession No. WP_011021232; 77.13% identical) and Bacteroidales bacterium Barb7 (Accession No. OAV73823; 71.25% identical), each with reference to SEQ ID NO:216. Further examples were reported in McRose et al. (2017).

Analogous to other NifD polypeptides which are functional, functional AnfD polypeptides are capable of functioning as the α protein structural component of the α₂β₂δ₂ heterohexameric nitrogenase with the β protein (AnfK) and the δ protein (AnfG), providing the catalytic complex binding FeFe-co for dinitrogen reduction.

As used herein, a “NifK polypeptide” means a polypeptide comprising amino acids whose sequence is at least 31% identical to the amino acid sequence provided as SEQ ID NO:3 and which comprises one or more of the conserved domains cd01974, TIGR01286, or cd01973 in which case the NifK polypeptide is in the subclass of VnfK polypeptides, or c102775 containing the TIGR02931 conserved domain in which case the NifK polypeptide is in the subclass of AnfK polypeptides. As used herein, NifK polypeptides include the VnfK polypeptides from iron-vanadium nitrogenase and the AnfK iron-binding polypeptides. A naturally occurring NifK polypeptide typically has a length of between 430 and 530 amino acids. A great number of NifK polypeptides have been identified and numerous sequences are available in publically available databases. For example, NifK polypeptides have been reported from Klebsiella michiganensis (Accession No. WP_049080161.1, 99% identical to SEQ ID NO:3), Raoultella ornithinolytica (WP_044347163.1, 96% identical), Klebsiella variicola (SBM87811.1, 94% identical), Kluyvera intermedia (WP_047370272.1, 89% identical), Rahnella aquatilis (WP_014333919.1, 82% identical), Tolumonas auensis (WP_012728880.1, 75% identical), Pseudomonas stutzeri (WP_011912506.1, 68% identical), Vibrio natriegens (WP_065303473.1, 65% identical), Azoarcus toluclasticus (WP_018989051.1, 54% identical), Frankia sp. (prf12106319A, 50% identical) and Methanosarcina acetivorans (WP_011021239.1, 31% identical). There are some examples of polypeptides in databases annotated as “NifK” which have less than 31% identity to SEQ ID NO:3 but do not contain any of the domains listed above and are therefore not included as NifK polypeptides herein. NifK polypeptides have been described and reviewed in Kim and Rees (1994), Eady (1996), Robson et al. (1989), Dilworth et al. (1988), Dilworth et al. (1993), Miller and Eady (1988), Igarashi and Seefeldt (2003), Fani et al. (2000) and Rubio and Ludden (2008).

NifK polypeptides of the iron-molybdenum subclass are a key subunit of nitrogenase complexes, being the β subunit of the α₂β₂ MoFe protein complex at the core of nitrogenase. As used herein, a functional NifK polypeptide is a NifK polypeptide which is capable of forming a functional nitrogenase protein complex together with the other required subunits, for example, NifD and NifH, and the FeMo or other cofactor. In a preferred embodiment, when aligned with the amino acid sequence SEQ ID NO:3, the amino acid sequence of the NifK polypeptide of the invention has at its C-terminus the amino acids DLVR (SEQ ID NO:58), the arginine being the C-terminal amino acid. That is, the NifK polypeptide and the NifK fusion polypeptide of the invention preferably has the same C-terminus as the native NifK polypeptides, i.e., it does not have an artificial addition to the C-terminus. Such preferred NifK polypeptides are better able to form a functional nitrogenase complex with NifD and NifH polypeptides.

NifK polypeptides of the iron-molybdenum subclass are a key subunit of nitrogenase complexes, being the β subunit of the α₂β₂ MoFe protein complex at the core of nitrogenase. As used herein, a functional NifK polypeptide is a NifK polypeptide which is capable of forming a functional nitrogenase protein complex together with the other required subunits, for example, NifD and NifH, and the FeMo or other cofactor. In a preferred embodiment, when aligned with the amino acid sequence SEQ ID NO:3, the amino acid sequence of the NifK fusion polypeptide and the cleaved NifK polypeptide of the invention have at its C-terminus the amino acids DLVR (SEQ ID NO:58), the arginine being the C-terminal amino acid. In other preferred embodiments, the amino acid sequence of the NifK fusion polypeptide and the cleaved NifK polypeptide of the invention have at its C-terminus the amino acid sequence DLIR (SEQ ID NO:239), DVVR (SEQ ID NO:240), DIIR (SEQ ID NO:241), DLTR (SEQ ID NO:242) or INVW (SEQ ID NO:243), which are typically not present in native AnfK sequences. The NifK polypeptide and the NifK fusion polypeptide of the invention, and the cleaved NifK polypeptide therefrom, preferably has the same C-terminus as a native NifK polypeptide, i.e., it does not have an artificial addition to the C-terminus, and it does not have any amino acids deleted from the C-terminus when aligned with a native NifK polypeptide. Such preferred NifK polypeptides are better able to form a functional nitrogenase complex with NifD and NifH polypeptides.

As used herein, an “AnfK polypeptide” is a polypeptide which is a member of the oxidoreductase nitrogenase conserved superfamily c102775, containing the TIGR02931 conserved domain, and having at least 54% amino acid sequence identity to the Azotobacter vinelandii AnfK polypeptide (SEQ ID NO:217; Accession No. WP_012703359) when measured along the full-length of SEQ ID NO:217. This amino acid sequence is used herein as the reference sequence for AnfK. TIGR02931AnfK represents the all-iron variant of the nitrogenase component I β-chain. As used herein, an AnfK polypeptide may be a NifK polypeptide, having at least 31% amino acid identity to SEQ ID NO:3. Other AnfK polypeptides are less homologous and are only 25-31% identical to SEQ ID NO:3 but are nevertheless included in AnfK polypeptides of the invention AnfK polypeptides do not include the molybdenum type NifK polypeptides and the vanadium type NifK polypeptides (VnfK). The AnfK fusion polypeptide and the cleaved AnfK polypeptide of the invention preferably have the same C-terminus as a native AnfK polypeptide, i.e., it does not have an artificial addition to the C-terminus, and it does not have any amino acids deleted from the C-terminus when aligned with a native AnfK polypeptide such as SEQ ID NO:217. In preferred embodiments, the amino acid sequence of the AnfK fusion polypeptide and the cleaved AnfK polypeptide of the invention has at its C-terminus the amino acid sequence LNVW (SEQ ID NO:244), LNTW (SEQ ID NO:245), LNMW (SEQ ID NO:246), LAMW (SEQ ID NO:247) or LSVW (SEQ ID NO:248). The amino acid sequences of AnfK polypeptides in the protein sequence database are usually annotated as an AnfK polypeptide. As of January 2020, there were 155 specific amino acid sequences in the protein database in the AnfK set, which were distinct from the molybdenum-type NifK and VnfK polypeptide sequences. Examples of naturally occurring AnfK polypeptides include AnfK polypeptides from Azomonas agilis (Accession No. WP_144571040; 91.34% identical), Clostridium sp. BL-8 (Accession No. WP_077859050; 78.35% identical), Lucifera butyrica (Accession No. WP_122630336; 62.34% identical) and Rhodoblastus acidophilus (Accession No. WP_088520366; 54% identical), each with reference to SEQ ID NO:217.

Analogous to other NifK polypeptides which are functional, functional AnfK polypeptides are capable of functioning as the β protein structural component of the α₂β₂δ₂ heterohexameric nitrogenase with the a protein (AnfD) and the δ protein (AnfG) to form the complex having the active site for dinitrogen reduction on FeFe-co.

A NifB polypeptide in naturally occurring bacteria is a protein which converts [4Fe-4S] clusters into NifB-co, an Fe—S cluster of higher nuclearity with a central C atom that serves as a precursor of FeMo-co, FeV-co and FeFe-co synthesis (Guo et al., 2016). NifB therefore catalyses the first committed step in the FeMo-co, FeV-co and FeFe-co synthesis pathways and is therefore essential for nitrogenase function. The NifB-co product of NifB is able to bind to the NifE-NifN complex and can be shuttled from NifB to NifE-NifN by the metallocluster carrier protein NifX.

As used herein, a “NifB polypeptide” means a polypeptide whose amino acid sequence comprises amino acids whose sequence is at least 27% identical to the amino acid sequence provided as SEQ ID NO:4. Most NifB polypeptides comprise one or more of the conserved domain TIGR01290, the NifB conserved domain cd00852, the NifX-NifB superfamily conserved domain c100252 and the Radical_SAM conserved domain cd01335. As used herein, NifB polypeptides include naturally occurring polypeptides which have been annotated as having NifB function but which do not have one of these domains. NifB polypeptides from Klebsiella, Azotobacter, Rhizobium, Bradyrhizobium and other bacteria have a C-terminal NifX-like extension, whereas most archeal NifB polypeptides lack the NifX-like domain and are referred to as “truncated NifB polypeptides”. A naturally occurring NifB polypeptide typically has a length of between 440 and 500 amino acids and the natural monomer has a molecular weight of about 50 kDa. A great number of NifB polypeptides have been identified and numerous sequences are available in publically available databases. For example, NifB polypeptides have been reported from Raoultella ornithinolytica (Accession No. WP_041145602.1, 91% identical to SEQ ID NO:4), Kosakonia radicincitans (WP_043953592.1, 80% identical), Dickeya chrysanthemi (WP_040003311.1, 76% identical), Pectobacterium atrosepticum (WP_011094468.1, 70% identical), Brenneria goodwinii (WP_048638849.1, 63% identical), Halorhodospira halophila (WP_011813098.1, 59% identical, lacking a NifX domain), Methanosarcina barkeri (WP_048108879.1, 50% identical, lacking a NifX domain), Clostridium purinilyticum (WP_050355163.1, 40% identical, lacking a NifX domain) and Desulfovibrio salexigens (WP_015850328.1, 27% identical). As used herein, a “functional NifB polypeptide” is a NifB polypeptide which is capable of forming NifB-co from [4Fe-4S] clusters. Functional NifB requires S-adenosyl-methionine (SAM) for its function. NifB polypeptides have been described and reviewed in Curatti et al. (2006) and Allen et al. (1995).

Boyd et al. (2011) investigated the phylogenetic relationship of Anf/Vnf/NifDKEN and NifB from 40 taxa and made the following conclusions: (1) Lateral gene transfer of the Nif cluster encoding a NifB lacking a C-terminal NifX domain occurred from a methanogen ancestor in the order Methanosarcinales to an anaerobic Firmicutes ancestor, where the two organisms coexisted in an anaerobic environment and where molybdenum was available, and (2) after this lateral gene transfer event, fusion of NifB and NifX occurred in the Firmicutes, from which the diazotrophic bacterial lineage evolved. The following evidence was provided to support this theory: (1) None of the methanogenic archaea (Methanococcales, Methanosarcinales and Methanobacteriales) have a NifB with a C-terminal NifX domain, (2) NifB sequences from Methanobacteriales and Methanococcales indicate early divergence from those of Methanosarcinales and Bacteria, and (3) some of the anaerobic Firmicutes, Chloroflexi and Proteobacteria that have a NifB without the C-terminal NifX domain diverged early from the Firmicute lineage, supposedly shortly after the Nif lateral gene transfer event.

To determine the presence or absence of a C-terminal NifX domain in NifB polypeptides, a NifB amino acid sequence can be aligned using Constraint-based Multiple Alignment Tool (COBALT, NCBI, www.ncbi.nlm.nih.gov/tools/cobalt/re_cobalt/cgi) with representative NifB sequences such as from Klebsiella michiganensis NifB (Accession No. P10930), Klebsiella michiganensis NifX (KZT46636.1), NifY (KZT46633.1), A. vinelandii NifX (AGK13791.1), NifY (AGK13792.1), NafY (AGK13761.1), and NifX/NifY/NafY/VnfX family protein (AGK14217.1). The ‘dinitrogenase FeMo-cofactor binding site’ (Pfam family PF02579) in each sequence can be identified by PfamScan (EMBL-EBI, www.ebi.ac.uk/Tools/pfa/pfamscan/), using the Pfam-A database with the expectation value set to 10.

The NifEN complex is a scaffold complex that is required for the correct assembly of dinitrogenase, functioning as the scaffold for NifB-co maturation into FeMo-co which process also requires NifH function, and is also structurally similar to the dinitrogenase (Fay et al., 2016). The NifEN complex is comprised of 2 subunits of each of NifE and NifN, respectively, forming a heterotetramer, here termed ENα₂β₂. A NifE polypeptide in naturally occurring bacteria is a polypeptide which is the a subunit of the ENα₂β₂ tetramer with the NifN polypeptide, and this ENα₂β₂ tetramer is required for FeMo-co synthesis and is proposed to function as a scaffold on which FeMo-co is synthesized.

As used herein, a “NifE polypeptide” means a polypeptide comprising amino acids whose sequence is at least 32% identical to the amino acid sequence provided as SEQ ID NO:5 and which comprises one or both of the domains TIGR01283 and PRK14478. Members of TIGR01283 domain protein family are also members of the superfamily c102775. A naturally occurring NifE polypeptide typically has a length of between 440 and 490 amino acids and the natural monomer has a molecular weight of about 50 kDa. A great number of NifE polypeptides have been identified and numerous sequences are available in publically available databases. For example, NifE polypeptides have been reported from Klebsiella michiganensis (Accession No. WP_049114606.1, 99% identical to SEQ ID NO:5), Klebsiella variicola (SBM87755.1, 92% identical), Dickeya paradisiaca (WP_012764127.1, 89% identical), Tolumonas auensis (WP_012728883.1, 75% identical), Pseudomonas stutzeri (WP_003297989.1, 69% identical), Azotobacter vinelandii (WP_012698965.1, 62% identical), Trichormus azollae (WP_013190624.1, 55% identical), Paenibacillus durus (WP_025698318.1, 50% identical), Sulfitricurvum kujiense (WP_013460149.1, 44% identical), Methanobacterium formicicum (AIS 31022.1, 39% identical), Anaeromusa acidaminophila (WP_018701501.1, 35% identical) and Megasphaera cerevisiae (WP_048514099.1, 32% identical). As used herein, a “functional NifE polypeptide” is a NifE polypeptide which is capable of forming a functional tetramer together with NifN such that the complex is capable of synthesizing FeMo-co. This synthesis of FeMo-co involves other polypeptides including NifH and NifB and may involve NifX. NifE polypeptides have been described and reviewed in Fay et al. (2016), Hu et al. (2005), Hu et al. (2006) and Hu et al. (2008).

A NifF polypeptide in naturally occurring diazotrophs is a flavodoxin which is an electron donor to NifH. As used herein, a “NifF polypeptide” means a polypeptide comprising amino acids whose sequence is at least 34% identical to the amino acid sequence provided as SEQ ID NO:6 and which comprises one or both of the flavodoxin long domain domain TIGR01752 and the flavodoxin FLDA domain found on Nif proteins from Azobacter and other bacterial genera PRK09267. NifF polypeptides encompass flavodoxins associated with pyruvate formate-lyase activation and cobalamin-dependent methionine synthase activity in non-nitrogen fixing bacteria but exclude other flavodoxins involved in broader functions. A naturally occurring NifF polypeptide typically has a length of between 160 and 200 amino acids and the natural monomer has a molecular weight of about 19 kDa. A great number of NifF polypeptides have been identified and numerous sequences are available in publically available databases. For example, NifF polypeptides have been reported from Klebsiella michiganensis (Accession No. WP_004122417.1, 99% identical to SEQ ID NO:6), Klebsiella variicola (WP_040968713.1, 85% identical), Kosakonia radicincitans (WP_035885760.1, 76% identical), Dickeya chrysanthemi (WP_039999438.1, 72% identical), Brenneria goodwinii (WP_048638838.1, 62% identical), Methylomonas methanica (WP_064006977.1, 56% identical), Azotobacter vinelandii (WP_012698862.1, 50% identical), Chlorobaculum tepidum (WP_010933399.1, 39% identical), Campylobacter showae (WP_002949173.1, 37% identical) and Azotobacter chromococcum (WP_039801725.1, 34% identical). As used herein, a “functional NifF polypeptide” is a NifF polypeptide which is capable of being an electron donor to a NifH polypeptide. NifF polypeptides have been described and reviewed in Drummond (1985).

As used herein, an “AnfG polypeptide” is a member of the nitrogenase conserved superfamily c103910 (pfam03139-AnfG), containing the TIGR02929 conserved domain, and having at least 42% amino acid sequence identity to the Azotobacter vinelandii AnfG polypeptide (SEQ ID NO:219; Accession No. WP_012703360) when measured along the full-length of SEQ ID NO:219. This amino acid sequence is used herein as the reference sequence for AnfG. TIGR02929 represents the all-iron variant of the nitrogenase component I 6-chain. AnfG polypeptides do not include the vanadium type NifG polypeptides (VnfG). The amino acid sequences of AnfG polypeptides in the protein sequence database are usually annotated as an AnfG polypeptide. As of January 2020, there were 150 specific amino acid sequences in the protein database in the AnfG set. Examples of naturally occurring AnfG polypeptides include AnfG polypeptides from Azomonas agilis (Accession No. WP_144571041; 84.73% identical), Firmicutes bacterium (Accession No. HBE76208; 70.37% identical), Sporomusa termitida (Accession No. WP_144349445; 68.75% identical), Rhodovulum viride (Accession No. WP_112317428; 57.14% identical) and Megasphaera cerevisiae (Accession No. WP_048515315; 42.86% identical), each with reference to SEQ ID NO:219.

Functional AnfG polypeptides are capable of functioning as the δ protein structural component of the α₂β₂δ₂heterohexameric nitrogenase.

A NifJ polypeptide in naturally occurring bacteria is a pyruvate:flavodoxin (ferredoxin) oxidoreductase which is an electron donor to NifH. As used herein, a “NifJ polypeptide” means a polypeptide comprising amino acids whose sequence is at least 40% identical to the amino acid sequence provided as SEQ ID NO:7 and which comprises the conserved domain TIGR02176. A naturally occurring NifJ polypeptide typically has a length of between 1100 and 1200 amino acids and the natural monomer has a molecular weight of about 128 kDa. A great number of NifJ polypeptides have been identified and numerous sequences are available in publically available databases. For example, NifJ polypeptides have been reported from Klebsiella michiganensis (Accession No. WP_024360006.1, 99% identical to SEQ ID NO:7), Raoultella ornithinolytica (WP_044347157.1, 95% identical), Klebsiella quasipneumoniae (WP_050533844.1, 92% identical), Kosakonia oryzae (WP_064566543.1, 82% identical), Dickeya solani (WP_057084649.1, 78% identical), Rahnella aquatilis (WP_014683040.1, 72% identical), Thermoanaerobacter mathranii (WP_013149847.1, 64% identical), Clostridium botulinum (WP_053341220.1, 60% identical), Spirochaeta africana (WP_014454638.1, 52% identical) and Vibrio cholerae (CSA83023.1, 40% identical). As used herein, a “functional NifJ polypeptide” is a NifJ polypeptide which is capable of being an electron donor to a NifH polypeptide. NifJ polypeptides have been described and reviewed in Schmitz et al. (2001).

A NifM polypeptide in naturally occurring bacteria is a polypeptide required for maturation of some but not all NifH polypeptides. In the absence of NifM, K. oxytoca NifH was present at only low levels in E. coli and yeast when expressed heterologously and was not able to donate electrons to NifD-NifK. As used herein, a “NifM polypeptide” means a polypeptide comprising amino acids whose sequence is at least 26% identical to the amino acid sequence provided as SEQ ID NO:8 and which comprises the domain TIGR02933. NifM polypeptides are homologous to peptidyl-prolyl cis-trans isomerases (PPIase), a group of enzymes that promote protein folding by catalysing the cis-trans isomerisation of proline imidic peptide bonds, having a PpiC-type domain, and appear to be accessory proteins for some NifH polypeptides, including at least some VnfH and AnfH polypeptides. A naturally occurring NifM polypeptide typically has a length of between 240 and 300 amino acids and the natural monomer has a molecular weight of about 30 kDa. A great number of NifM polypeptides have been identified and numerous sequences are available in publically available databases. For example, NifM polypeptides have been reported from Klebsiella oxytoca (Accession No. WP_064342940.1, 99% identical to SEQ ID NO:8), Klebsiella michiganensis (WP_004122413.1, 97% identical), Raoultella ornithinolytica (WP_044347181.1, 85% identical), Klebsiella variicola (WP_063105800.1, 75% identical), Kosakonia radicincitans (WP_035885759.1, 59% identical), Pectobacterium atrosepticum (WP_011094472.1, 42% identical), Brenneria goodwinii (WP_048638837.1, 33% identical), Pseudomonas aeruginosa PAO1 (CAA75544.1, 28% identical), Marinobacterium sp. AK27 (WP_051692859.1, 27% identical) and Teredinibacter turnerae (WP_018415157.1, 26% identical). As used herein, a “functional NifM polypeptide” is a NifM polypeptide which is capable of complexing with a NifH polypeptide for maturation of the NifH polypeptide. NifM polypeptides have been described and reviewed in Petrova et al. (2000).

A NifN polypeptide in naturally occurring bacteria is the β subunit of the ENα₂β₂ tetramer with the NifE polypeptide, and the ENα₂β₂ tetramer is required for FeMo-co synthesis and is proposed to function as a scaffold on which FeMo-co is synthesized. As used herein, a “NifN polypeptide” means (i) a polypeptide comprising amino acids whose sequence is at least 76% identical to the sequence provided as SEQ ID NO:9 and/or (ii) a polypeptide comprising amino acids whose sequence is at least 34% identical to the sequence provided as SEQ ID NO:9 and which comprises one or more of the conserved domains TIGR01285, cd01966 and PRK14476. NifN is related in structure to the molybdenum-iron protein B chain NifK. Polypeptides comprising the conserved TIGR01285 covers most examples of NifN polypeptides but excludes some NifN polypeptides, such as the putative NifN of Chlorobium tepidum, and therefore the definition of NifN is not limited to polypeptides comprising the conserved TIGR01285 domain. Members of PRK14476 domain protein family are also members of the superfamily c102775. A naturally occurring NifN polypeptide typically has a length of between 410 and 470 amino acids, although when fused naturally to NifE it may have about 900 amino acid residues, and the natural monomer has a molecular weight of about 50 kDa. A great number of NifN polypeptides have been identified and numerous sequences are available in publically available databases. For example, NifN polypeptides have been reported from Klebsiella oxytoca (Accession No. WP_064391778.1, 97% identical to SEQ ID NO:9), Kluyvera intermedia (WP_047370268.1, 80% identical), Rahnella aquatilis (WP_014683026.1, 70% identical), Brenneria goodwinii (WP_048638830.1, 65% identical), Methylobacter tundripaludum (WP_027147663.1, 46% identical), Calothrix parietina (WP_015195966.1, 41% identical), Zymomonas mobilis (WP_023593609.1, 37% identical), Paenibacillus massiliensis (WP_025677480.1, 35% identical) and Desulfitobacterium hafniense (WP_018306265.1, 34% identical). As used herein, a “functional NifN polypeptide” is a NifN polypeptide which is capable of forming a functional tetramer together with NifE such that the complex is capable of synthesizing FeMo-co. NifN polypeptides have been described and reviewed in Fay et al. (2016), Brigle et al. (1987), Fani et al. (2000), and Hu et al. (2005).

A NifQ polypeptide in naturally occurring bacteria is a polypeptide involved in FeMo-co synthesis, probably in early MoO₄²⁻ processing. The conserved C-terminal cysteine residues may be involved in metal binding. As used herein, a “NifQ polypeptide” means a polypeptide comprising amino acids whose sequence is at least 34% identical to the amino acid sequence provided as SEQ ID NO:10 and which is a member of the CL04826 domain protein family and a member of the pfam04891 domain protein family. A naturally occurring NifQ polypeptide typically has a length of between 160 and 250 amino acids, although they may be as long as 350 amino acid residues, and the natural monomer has a molecular weight of about 20 kDa. A great number of NifQ polypeptides have been identified and numerous sequences are available in publically available databases. For example, NifQ polypeptides have been reported from Klebsiella oxytoca (Accession No. WP_064391765.1, 95% identical to SEQ ID NO:10), Klebsiella variicola (CTQ06350.1, 75% identical), Kluyvera intermedia (WP_047370257.1, 63% identical), Pectobacterium atrosepticum (WP_043878077.1, 59% identical), Mesorhizobium metallidurans (WP_008878174.1, 46% identical), Rhodopseudomonas palustris (WP_011501504.1, 42% identical), Paraburkholderia sprentiae (WP_027196569.1, 41% identical), Burkholderia stabilis (GAU06296.1, 39% identical) and Cupriavidus oxalaticus (WP_063239464.1, 34% identical). As used herein, a “functional NifQ polypeptide” is a NifQ polypeptide which is capable of processing MoO₄²⁻. NifQ polypeptides have been described and reviewed in Allen et al. (1995) and Siddavattam et al. (1993).

A NifS polypeptide in naturally occurring bacteria is a cysteine desulfurase involved in iron-sulfur (FeS) cluster biosynthesis e.g. which is involved in mobilisation of sulfur for Fe—S cluster synthesis and repair. As used herein, a “NifS polypeptide” means (i) a polypeptide comprising amino acids whose sequence is at least 90% identical to the amino acid sequence provided as SEQ ID NO:19 and/or (ii) a polypeptide comprising amino acids whose sequence is at least 36% identical to the sequence provided as SEQ ID NO:19 and which comprises one or both of the conserved domains TIGR03402 and COG1104. The TIGR03402 domain protein family includes a Glade nearly always found in extended nitrogen fixation systems plus a second Glade more closely related to the first than to IscS and also part of NifS-like/NifU-like systems. The TIGR03402 domain protein family does not extend to a more distant Glade found in the epsilon proteobacteria such as Helicobacter pylori, also named NifS in the literature, built instead in TIGR03403. The COG1104 domain protein family includes cysteine sulfinate desulfinase/cysteine desulfurase or related enzymes. Some NifS polypeptides include the asparate aminotransferase domain c118945. A naturally occurring NifS polypeptide typically has a length of between 370 and 440 amino acids and the natural monomer has a molecular weight of about 43 kDa. A great number of NifS polypeptides have been identified and numerous sequences are available in publically available databases. For example, NifS polypeptides have been reported from Klebsiella michiganensis (Accession No. WP_004138780.1, 99% identical to SEQ ID NO:19), Raoultella terrigena (WP_045858151.1, 89% identical), Kluyvera intermedia (WP_047370265.1, 80% identical), Rahnella aquatilis (WP_014333911.1, 73% identical), Agarivorans gilvus (WP_055731597.1, 64% identical), Azospirillum brasilense (WP_014239770.1, 60% identical), Desulfosarcina cetonica (WP_054691765.1, 55% identical), Clostridium intestinale (WP_021802294.1, 47% identical), Clostridiisalibacter paucivorans (WP_026894054.1, 36% identical) and Bacillus coagulans (WP_061575621.1, 42% identical and which is in COG1104). As used herein, a “functional NifS polypeptide” is a NifS polypeptide which is capable of functioning in iron-sulfur (FeS) cluster biosynthesis and/or repair. NifS polypeptides have been described and reviewed in Clausen et al. (2000), Johnson et al. (2005), Olson et al. (2000) and Yuvaniyama et al. (2000).

A NifU polypeptide in naturally occurring bacteria is a molecular scaffold polypeptide involved in iron-sulfur (FeS) cluster biosynthesis for nitrogenase components. As used herein, a “NifU polypeptide” means a polypeptide comprising amino acids whose sequence is at least 31% identical to the sequence provided as SEQ ID NO:12 and which comprises the domain TIGR02000. Members of the TIGR02000 domain protein family are specifically involved in nitrogenase maturation. NifU comprises an N-terminal domain (pfam01592) and a C-terminal domain (pfam01106). Three different but partially homologous Fe—S cluster assembly systems have been described: Isc, Suf, and Nif. The Nif system, of which NifU is a part, is associated with donation of an Fe—S cluster to nitrogenase in a number of nitrogen-fixing species. Isc and Suf homologs with an equivalent domain architecture from Helicobacter and Campylobacter are excluded from the definition of NifU herein. NifU, therefore, is specific for NifU polypeptides involved in nitrogenase maturation. Members of the related TIGR01999 domain protein family which are IscU proteins (from for example, Escherichia. coli and Saccharomyces cerevisiae and Homo sapiens) that comprise a homolog of the N-terminal region of NifU are also excluded from the definition of NifU herein. A naturally occurring NifU polypeptide typically has a length of between 260 and 310 amino acids and the natural monomer has a molecular weight of about 29 kDa. A great number of NifU polypeptides have been identified and numerous sequences are available in publically available databases. For example, NifU polypeptides have been reported from Klebsiella michiganensis (Accession No. WP_049136164.1, 97% identical to SEQ ID NO:12), Klebsiella variicola (WP_050887862.1, 90% identical), Dickeya solani (WP_057084657.1, 80% identical), Brenneria goodwinii (WP_048638833.1, 73% identical), Tolumonas auensis (WP_012728889.1, 66% identical), Agarivorans gilvus (WP_055731596.1, 58% identical), Desulfocurvus vexinensis (WP_028587630.1, 54% identical), Rhodopseudomonas palustris (WP_044417303.1, 49% identical), Helicobacter pylori (WP_001051984.1, 31% identical) and Sulfitrovum sp. PC08-66 (KIM05011.1, 31% identical). As used herein, a “functional NifU polypeptide” is a NifU polypeptide which is capable of functioning as a molecular scaffold polypeptide involved in iron-sulfur (FeS) cluster biosynthesis. NifU polypeptides have been described and reviewed in Hwang et al. (1996), Mühlenhoff et al. (2003) and Ouzounis et al. (1994).

NifS is a pyridoxal phosphate (PLP, vitamin B6) dependent cysteine desulfurase which generates the inorganic sulphide required for Fe—S cluster synthesis from cysteine. The reaction produces alanine as a byproduct. The reaction proceeds via a protein-bound cysteine persulfide intermediate that is formed by the nucleophilic attack of a highly conserved cysteine residue (Cys325 in Azotobacter vinelandii) on the cysteine-PLP adduct (Zheng et al., 1994). The sulphide is the provided to NifU for the sequential formation of [Fe₂S₂] and [Fe₄S₄] clusters. The NifS enzyme functions in bacteria as a homodimer.

NifU provides a scaffold for [Fe₄S₄] cluster formation, functioning as a homodimer. The NifU polypeptide contains three domains, namely a N-terminal scaffolding domain, a central domain and a C-terminal scaffolding domain (Smith et al., 2005). The N-terminal domain has a high sequence homology to IscU proteins from bacteria and Isu proteins from eukaryotes, while the C-terminal domain is homologous to Nfu proteins found in mitochondria and chloroplasts. The central domain contains one permanent redox-active [Fe₂S₂]²⁺ cluster per NifU subunit which, due to its stability, is thought not to be transferred to other Nif proteins. That cluster is thought to be coordinated by four conserved cysteine residues (Cys137, 139, 172 and 175 in A. vinelandii NifU) (Fu et al., 1994). In bacteria, NifU forms a homodimer and its N-terminal domain can bind one [Fe₂S₂]²⁺ cluster per monomer. The [Fe₂S₂] clusters in the monomers can be reductively fused to form one [Fe₄S₄] cluster per NifU dimer. A pair of [Fe₄S₄] clusters are then delivered from NifU to NifB and processed into an 8Fe core on NifB which is subsequently used for the synthesis of FeMoco. In a divergent pathway for the Fe—S clusters, one [Fe₄S₄] cluster bound to either the N-terminal or C-terminal scaffolding domain of NifU is transferred to apo-NifH for maturation of nitrogenase reductase, the NifH protein (Smith et al., 2005). It has been proposed that NifU also donates two [Fe₄S₄] clusters to a NifD-NifK protein complex (designated herein as stage 0 D-K), and that NifH condenses that pair of clusters into a mature P-cluster [Fe₈—S₇] (Dos Santos et al., 2004). These N-terminal clusters are thought to be extremely labile and are not retained during purification (Smith et al., 2005). The C terminal domain can hold one [Fe₄S₄] cluster per monomer. In contrast to the N-terminal cluster, the assembly of the C terminal [Fe₄S₄] cluster is rapid and no intermediate [Fe₂S₂] cluster has been detected (Smith et al., 2005). The C-terminal clusters are more stable than the N-terminal clusters and can be retained during purification. However, upon reduction with dithionite, the C-terminal clusters are rapidly degraded (Smith et al., 2005). Using cysteine to alanine mutations in NifU, Dos Santos and colleagues showed that both the N- and C-terminal clusters can be transferred to apo-NifH.

López-Torrejón et al. (2016) reported that a NifH protein capable of donating electrons to holoNifD-NifK can be generated within yeast mitochondria via the expression of both NifH and NifM. These authors found that, in the yeast cells, NifS and NifU were not required for the generation of NifH protein with this function. They concluded that endogenous iron sulphur cluster assembly pathways in the yeast cells, presumably mitochondrial-located Nfs1 and Nfu1 proteins which are related proteins in yeast, were capable of donating [Fe₄S₄] clusters to NifH. It therefore is possible that NifS and NifU will not be required for reconstituting the NifH protein, the Fe-protein or dinitrogenase reductase in yeast, but NifS and NifU may be required for NifB and/or NifD-NifK maturation and function. Whether plant mitochondria have similar endogenous ability for forming sufficient [Fe₄S₄] clusters for nitrogenase activity is unknown.

A NifV polypeptide in naturally occurring bacteria is a homocitrate synthase (EC 2.3.3.14), producing homocitrate by the transfer of the acetyl group from acetyl-coenzyme A (acetyl-CoA) to 2-oxoglutarate. Homocitrate is then used in the synthesis of FeMo-co, FeV-co and FeFe-co. As used herein, a “NifV polypeptide” means a polypeptide comprising amino acids whose sequence is at least 39% identical to the amino acid sequence provided as SEQ ID NO:13 and which comprises one or both of the domains TIGR02660 and DRE TIM. Members of the TIGR02660 domain protein family are homologous to enzymes that include 2-isopropylmalate synthase, (R)-citramalate synthase, and homocitrate synthase associated with processes other than nitrogen fixation. The cd07939 domain protein family also includes the NifV proteins of Heliobacterium chlorum and Gluconacetobacter diazotrophicus, which appear to be orthologous to FrbC. This family belongs to the DRE-TIM metallolyase superfamily. DRE-TIM metallolyases include 2-isopropylmalate synthase (IPMS), alpha-isopropylmalate synthase (LeuA), 3-hydroxy-3-methylglutaryl-CoA lyase, homocitrate synthase, citramalate synthase, 4-hydroxy-2-oxovalerate aldolase, re-citrate synthase, transcarboxylase 5S, pyruvate carboxylase, AksA, and FrbC. These members all share a conserved triose-phosphate isomerase (TIM) barrel domain consisting of a core beta(8)-alpha(8) motif with the eight parallel beta strands forming an enclosed barrel surrounded by eight alpha helices. The domain has a catalytic center containing a divalent cation-binding site formed by a cluster of invariant residues that cap the core of the barrel. In addition, the catalytic site includes three invariant residues—an aspartate (D), an arginine (R), and a glutamate (E)—which is the basis for the domain name “DRE-TIM”. A naturally occurring NifV polypeptide typically has a length of between 360 and 390 amino acids, although some members are about 490 amino acid residues in length, and the natural monomer has a molecular weight of about 41 kDa. A great number of NifV polypeptides have been identified and numerous sequences are available in publically available databases. For example, NifV polypeptides have been reported from Klebsiella michiganensis (Accession No. WP_049083341.1, 95% identical to SEQ ID NO:13), Raoultella ornithinolytica (WP_045858154.1, 86% identical), Kluyvera intermedia (WP_047370264.1, 81% identical), Dickeya dadantii (WP_038912041.1, 70% identical), Brenneria goodwinii (WP_048638835.1, 59% identical), Magnetococcus marinus (WP_011712856.1, 46% identical), Sphingomonas wittichii (WP_037528703.1, 43% identical), Frankia sp. EI5c (OAA29062.1, 41% identical) and Clostridium sp. Maddingley MBC34-26 (EKQ56006.1, 39% identical). As used herein, a “functional NifV polypeptide” is a NifV polypeptide which is capable of functioning as a homocitrate synthase. NifV polypeptides have been described and reviewed in Hu et al. (2008), Lee et al. (2000), Masukawa et al. (2007) and Zheng et al. (1997).

NifX polypeptide in Azotobacter vinelandii binds NifB-co (Fe₆—S₉—C), which is passed on to NifE-NifN for FeMo-co assembly (Hernandez et al., 2007). It has also been shown to exchange VK-clusters (Fe₈—S₉—C or Mo—Fe₇—S₉—C, Jimenez-Vincente et al., 2015) between NifE-NifN, suggesting its role as a transient reservoir for FeMo-co precursors. Hernandez et al. (2007) reported that NifX may act as a chaperone that stabilises the NifE-NifN or NifD-NifK complexes during transfer of FeMo-co to apo-NifD-NifK, and/or reposition the proteins in a favorable orientation for FeMoco transfer and so act to regulate FeMoco synthesis. Activation of apo-NifD-NifK by exogenous FeMo-co with dinitrogenase complexes extracted from A. vinelandii mutants deficient in different accessory protein combinations of NifY/NafY/NifX indicated that NifX can also assist in FeMo-co insertion of apo-NifD-NifK (Rubio et al., 2002). This additional function of NifX may be responsible for the retention of acetylene reduction activity in the Klebsiella ΔnifY mutant shown by Homer et al. (1993).

A NifX polypeptide in naturally occurring bacteria is a polypeptide which is involved in FeMo-co synthesis, at least assisting in transferring FeMo-co precursors from NifB to NifE-NifN or FeMo-co to NifD-NifK. As used herein, a “NifX polypeptide” means a polypeptide comprising amino acids whose sequence is at least 29% identical to the amino acid sequence provided as SEQ ID NO:14 and which comprises one or both of the conserved domains TIGR02663 and cd00853. NifX is included in a larger family of iron-molybdenum cluster-binding proteins that includes some NifB sequences and

NifY, in that NifX, NafY and the C-terminal region of some NifB polypeptides all comprise the pfam02579 domain, and each are involved in the synthesis of one or more or all of FeMo-co, FeV-co or FeFe-co. Other NifB polypeptides, specifically from methanogenic archaea and some anaerobic firmicutes, lack a NifX-like domain (Boyd et al., 2011), including NifB from H. halophila, M barkeri and C. purinilyticum mentioned above. Some NifX polypeptides have been annotated in databases as NifY, and vice versa. A naturally occurring NifX polypeptide, produced on its own rather than as a natural fusion as part of a NifB polypeptide, typically has a length of between 110 and 160 amino acids and the natural monomer has a molecular weight of about 15 kDa. A great number of NifX polypeptides have been identified and numerous sequences are available in publically available databases. For example, NifX polypeptides have been reported from Klebsiella michiganensis (Accession No. WP_049070199.1, 97% identical to SEQ ID NO:14), Klebsiella oxytoca (WP_064342937.1, 97% identical), Raoultella ornithinolytica (WP_044347173 0.1, 91% identical), Klebsiella variicola (WP_044612922.1, 83% identical), Kosakonia radicincitans (WP_043953583.1, 75% identical), Dickeya chrysanthemi (WP_039999416.1, 68% identical), Rahnella aquatilis (WP_047608097.1, 58% identical), Azotobacter chroococcum (WP_039800848.1, 34% identical), Beggiatoa leptomitiformis (WP_062149047.1, 33% identical) and Methyloversatilis discipulorum (WP_020165972.1, 29% identical). As used herein, a “functional NifX polypeptide” is a NifX polypeptide which is capable of transferring FeMo-co precursors from NifB to NifE-NifN. NifX polypeptides have been described and reviewed in Allen et al. (1994) and Shah et al. (1999).

A NifY polypeptide in naturally occurring bacteria is a polypeptide which is involved in FeMo-co synthesis, at least assisting in transferring FeMo-co precursors from NifB to NifE-NifN. As used herein, a “NifY polypeptide” means a polypeptide comprising amino acids whose sequence is at least 34% identical to the amino acid sequence provided as SEQ ID NO:15 and which comprises one or both of the conserved domains TIGR02663 and cd00853. NifY is included in a larger family of iron-molybdenum cluster-binding proteins that includes NifB and NifX, in that NifX, NafY and the C-terminal region of NifB all comprise the pfam02579 domain, and each are involved in the synthesis of FeMo-co. A great number of NifY polypeptides have been identified and numerous sequences are available in publically available databases. For example, NifY polypeptides have been reported from Klebsiella michiganensis (Accession No. WP_049089500.1, 99% identical to SEQ ID NO:15), Klebsiella oxytoca (WP_064342935.1, 98% identical), Klebsiella quasi pneumoniae (WP_044524054.1, 90% identical), Klebsiella variicola (WP_049010739.1, 81% identical), Kluyvera intermedia (WP_047370270.1, 69% identical), Dickeya chrysanthemi (WP_039999411.1, 62% identical), Serratia sp. ATCC 39006 (WP_037382461.1, 57% identical), Rahnella aquatilis (WP_014683024.1, 47% identical), Pseudomonas putida (AEX25784.1, 37% identical) and Azotobacter vinelandii (WP_012698835.1, 34% identical). As used herein, a “functional NifY polypeptide” is a NifY polypeptide which is capable of transferring FeMo-co precursors from NifB to NifE-NifN.

When isolated from NUB or NifB-NifE mutant strains of either K. oxytoca or A. vinelandii, apo-NifD-NifK was associated with an additional polypeptide termed the γ protein (Paustian et al, 1990; Homer et al., 1993), forming a heterohexamer with NifD and NifK polypeptides (α₂β₂γ₂). In K. oxytoca, the third polypeptide was encoded by the NifY gene (Homer et al., 1993) and the addition of purified FeMo-co to purified heterohexamer α₂β₂γ₂ complex was sufficient to yield catalytically active nitrogenase. Addition of FeMo-co resulted in dissociation of NifY from the complex with formation of the holoenzyme (α₂β₂). In A. vinelandii, the third polypeptide was encoded by the NafY gene (nitrogenase associated factor Y; Accession No. AGK13761, Rubio et al., 2002) which was different but related to the product of the NifY gene in A. vinelandii (Accession No. AGK13792). The third polypeptide in each case was thought to be involved in assisting in the insertion of FeMo-co to form the active enzyme. This was supported by the ability of NafY and NifY to bind FeMo-co (Homer et al., 1995).

A. vinelandii NifY and NafY bind to apo-NifD-NifK, at different stages of NifD-NifK holoenzyme maturation, to either α-Cys²⁷⁵ or α-His⁴⁴² of NifD, both amino acid residues of which covalently anchor FeMo-co (Jimenez-Vincente et al., 2018). That is, NifY and NafY do not bind to apo-NifD-NifK simultaneously. The order of binding of NifY and NafY to apo-NifD-NifK is currently unknown. Dissociation of NifY from NifD-NifK upon FeMo-co insertion has been demonstrated for K. oxytoca nitrogenase (Homer et al., 1993) and NafY from NifD-NifK upon FeMo-co insertion for A. vinelandii (Homer et al., 1995). NafY is also thought to bind FeMo-co through His¹²¹ and possibly NifB-co as well, suggesting its role as a FeMo-co or FeMo-co precursor insertase (Rubio et al., 2004). A. vinelandii NifY seems to be functionally redundant based on lack of a phenotype in ΔnifY mutants (Rubio et al., 2002) and NafY is proposed to be the primary accessory protein to apo-NifD-NifK that supports FeMo-co insertion. On the other hand, Klebsiella species do not have a NafY gene and only have NifY to support FeMo-co insertion into apo-NifD-NifK, although a Klebsiella ΔnifY mutant still retained 60% of acetylene reduction activity (Homer et al., 1993). This retention of function indicated presence of another accessory protein in Klebsiella that could partially cover NifY function in its absence, such as NifX as described above.

As used herein, a “NafY polypeptide” means a polypeptide comprising amino acids whose sequence is at least 50% identical to the sequence provided as SEQ ID NO:238 (A. vinelandii NafY, Accession No. AGK13761, 243aa) along its full-length and which comprises the conserved domain pfam16844. This domain of about 91 amino acid residues in length is found by itself in some members and in the amino terminal half of longer NafY proteins. This region is negatively charged and appears to function for recognising and interacting with apo-NifD-NifK. A naturally occurring NafY polypeptide typically has a length of between 230 and 250 amino acids and the natural monomer has a molecular weight of ˜25-28 kDa. A great number of NafY polypeptides have been identified and numerous sequences are available in publically available databases; some have been annotated as NifX polypeptides because of the relatedness of NafY and NifX sequences. For example, NafY polypeptides have been reported from Azotobacter beijerinckii (WP_090728988, 93% identical to SEQ ID NO:238), Pseudomonas stutzeri, (WP_011912501, 69% identical), Halomonas endophytica (WP_102654474, 68% identical), Pseudomonas linyingensis (WP_090313081, 67% identical), Acidihalobacter prosperus (WP_038093031, 56% identical), Oscillatoriales cyanobacterium (WP_009769409, 50% identical) As used herein, a “functional NafY polypeptide” is a NafY polypeptide which is capable of binding to apo-NifD-NifK and to FeMo-co. The three-dimensional structure of NafY polypeptide from A. vinelandii and a comparison and distinction of NafY and NifY, NifX, VnfX and NifB polypeptide sequences was reported in Dyer et al. (2003).

A NifZ polypeptide in naturally occurring bacteria is a polypeptide which is involved in Fe—S cluster synthesis, specifically functioning in the coupling of a second Fe₄S₄ pair in the formation of the second P-cluster of the MoFe protein. NifZ is thought to act as a chaperone that induces a conformational change in at least the second half of apo-MoFe protein, allowing for the formation of the second P-cluster together with NifH. Deletion of NifZ in A. vinelandii decreased MoFe protein activity by 66% but had no effect on NifH activity. As used herein, a “NifZ polypeptide” means a polypeptide comprising amino acids whose sequence is at least 28% identical to the sequence provided as SEQ ID NO:16 and which comprises the conserved domain pfam04319. This domain of about 75 amino acid residues is found in isolation in some members and in the amino terminal half of the longer NifZ proteins. A naturally occurring NifZ polypeptide typically has a length of between 70 and 150 amino acids and the natural monomer has a molecular weight of about 9 to about 16 kDa. A great number of NifZ polypeptides have been identified and numerous sequences are available in publically available databases. For example, NifZ polypeptides have been reported from Klebsiella michiganensis (Accession No. WP_057173223.1, 93% identical to SEQ ID NO:16), Klebsiella oxytoca (WP_064342939.1, 95% identical), Klebsiella variicola (WP_043875005.1, 77% identical), Kosakonia radicincitans (WP_043953588.1, 67% identical), Kosakonia sacchari (WP_065368553.1, 58% identical), Ferriphaselus amnicola (WP_062627625.1, 47% identical), Paraburkholderia xenovorans (WP_011491838.1, 41% identical), Acidithiobacillus ferrivorans (WP_014029050.1, 35% identical) and Bradyrhizobium oligotrophicum (WP_015665422.1, 28% identical). As used herein, a “functional NifZ polypeptide” is a NifZ polypeptide which is capable of coupling a Fe₄S₄ cluster in Fe—S cluster synthesis. NifZ polypeptides have been described and reviewed in Cotton (2009) and Hu et al. (2004).

A NifW polypeptide in naturally occurring bacteria is a polypeptide which associates with NifZ polypeptide to form higher order complexes (Lee et al., 1998), and is involved in MoFe protein (NifD-NifK) synthesis or activity. NifW and NifZ appear to be involved in the formation or accumulation of MoFe protein (Paul and Merrick, 1987). As used herein, a “NifW polypeptide” means a polypeptide whose amino acid sequence comprises amino acids whose sequence is at least 28% identical to the amino acid sequence provided as SEQ ID NO:17 and which comprises the conserved NifW superfamily protein domain, architecture ID number 10505077 and is in Pfamily PF03206. A number of NifW polypeptides have been identified and numerous sequences are available in publically available databases. For example, NifW polypeptides have been reported from Klebsiella oxytoca (Accession No. WP_064342938.1, 98% identical to SEQ ID NO:17), Klebsiella michiganensis (WP_049080155.1, 94% identical), Enterobacter sp. 10-1 (WP_095103586.1, 90% identical), Klebsiella quasipneumoniae (WP_065877373.1, 81% identical), Pectobacterium polaris (WP_095699971.1, 69% identical), Dickeya paradisiaca (WP_012764136.1, 58% identical), Brenneria goodwinii (WP_053085547.1, 36% identical), Aquaspirillum sp. LM1 (WP_077299824.1, 44% identical), Candidatus Muproteobacteria bacterium RBG1664_10 (00140729, 34% identical), Azotobacter vinelandii (AC076430.1, 32% identical) and Methylocaldum marinum (BBA37427.1, 28% identical). As used herein, a “functional NifW polypeptide” is a NifW polypeptide which promotes or enhances one or more of the formation, accumulation or activity of MoFe protein. A functional NifW may interact with NifZ and/or play a role in the oxygen protection of the MoFe-protein (Gavini et al., 1998).

Most organisms including both bacteria and eukaryotes such as plants have numerous ferredoxins. For example, there are 15 or 16 proteins annotated as ferredoxin or ferredoxin-like in the A. vinelandii DJ and CA genomes, respectively. As used herein, a “ferredoxin polypeptide” is an electron carrier protein having one or two iron-sulfur clusters of the [2Fe-2S], [3Fe-4S] and/or [4Fe-4S] type that form their reactive centers, see review by Matsubara and Saeki (1992). They are involved in a variety of metabolic processes, including ferredoxin polypeptides which are involved in nitrogen fixation, generally of lower molecular weight than those not involved in nitrogenase. Based on the wide diversity of ferredoxins in most cells and the variations observed in several studies on the compatibility or specificity of different ferredoxins in complementing the function of FdxN for NifB-co synthesis (Yates, 1972; Jimenez-Vincente et al., 2014), ferredoxins including ones such as FdxN are best defined based on the presence of the iron-sulfur clusters and their function rather than on amino acid identity to a standard sequence such as A. vinelandii FdxN (SEQ ID NO:232; Accession No. WP_012703542). As used herein, a “FdxN polypeptide” is a ferredoxin or ferredoxin-like polypeptide which functions for donating electrons to mature dinitrogenase reductase NifH and/or for NifB-co synthesis for nitrogenase and/or serves as an intermediate carrier of [4Fe-4S] clusters. FdxN may function by donating electrons to mature dinitrogenase reductase NifH which then transfers the electrons to NifD-NifK heterohexamer (see Yang et al., 2017; Rhizobium japonicum FdxN, Carter et al., 1980; R. meliloti FdxN, Riedel et al., 1995; Rhodobacter capsulatus FdxN, Jouanneau et al., 1995), or donating electrons to NifB polypeptide for NifB-co synthesis (A. vinelandii: Jimenez-Vincente et al., 2014), or serves as an intermediate carrier of [4Fe-4] clusters (A. vinelandii: Buren et al., 2019), or a combination of any of these functions.

Representative examples of FdxN polypeptides include the following, identified by searching the non-redundant protein database using SEQ ID NO:232 as query in BLASTP and showing percentage identity to that sequence: Pseudomonas syringae (WP_065835964.1, 85.87%), Candidatus Thiodiazotropha endolucinida (WP_069124666.1, 70.65%), Uliginosibacterium sp. TH139 (WP_101942980, 64.47%), Klebsiella michiganensis (WP_049076934.1, 44.26%), Escherichia coli (WP_072048756.1, 44.26%), Rhizobium leguminosarum (WP_130674512.1, 43.86%) and Flavobacterium alvei (WP_103805005.1, 28.57%).

### Sequence Identity and Substitutions

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 30%, more preferably at least 35%, more preferably at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides defined herein can be prepared by introducing appropriate nucleotide changes into a nucleic acid defined herein, or by in vitro synthesis of the desired polypeptide. Such mutants include for example, one or more amino acid deletions, insertions, or substitutions. A combination of deletion, insertion and substitution mutations can be made to arrive at the final construct, provided that the final polypeptide product possesses the desired characteristics. Preferred amino acid sequence mutants have only one, two, three, four or less than 10 amino acid changes relative to the reference wildtype polypeptide.

Mutant (altered) polypeptides can be prepared using any technique known in the art, for example, using directed evolution or rational design strategies (see below). Products derived from mutated/altered DNA can readily be screened using techniques described herein to determine if their expression in a plant alters its phenotype relative to a corresponding wild-type plant, for example, if their expression results in increased yield, biomass, growth rate, vigor, nitrogen gain derived from biological nitrogen fixation, nitrogen use efficiency, abiotic stress tolerance, and/or tolerance to nutrient deficiency relative to the corresponding wild-type plant.

In designing amino acid sequence mutants, the location of the mutation site and the nature of the mutation will depend on characteristic(s) to be modified. The sites for mutation can be modified individually or in series for example, by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. Where it is desirable to maintain a certain activity it is preferable to make no, or only conservative substitutions, at amino acid positions which are highly conserved in the relevant protein family. Examples of conservative substitutions are shown in Table 1 under the heading of “exemplary substitutions”.

In a preferred embodiment a mutant/variant polypeptide has one or two or three or four conservative amino acid changes when compared to a naturally occurring polypeptide. Details of conservative amino acid changes are provided in Table 1. In a preferred embodiment, the changes are not in one or more of the motifs or domains which are highly conserved between the different polypeptides of the invention. As the skilled person would be aware, such minor changes can reasonably be predicted not to alter the activity of the polypeptide when expressed in a recombinant cell.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

The primary amino acid sequence of a polypeptide of the invention can be used to design variants/mutants thereof based on comparisons with closely related polypeptides. As the skilled person will appreciate, residues highly conserved amongst closely related proteins are less likely to be able to be altered, especially with non-conservative substitutions, and activity maintained than less conserved residues (see above). A more stringent test to identify conserved amino acid residues is to align more distantly related polypeptides of the same function. Highly conserved residues should be maintained in order to retain function, whereas non-conserved residues are more amenable to substitutions or deletion while maintaining function.

Also included within the scope of the invention are polypeptides of the present invention which are differentially modified during or after synthesis in a cell, e.g., by glycosylation, acetylation, phosphorylation or proteolytic cleavage.

### Rational Design

A protein can be designed rationally, on the basis of known information about protein structure and folding. This can be accomplished by design from scratch (de novo design) or by redesign based on native scaffolds (see, for example, Hellinga, 1997; and Lu and Berry, Protein Structure Design and Engineering, Handbook of Proteins 2, 1153-1157 (2007)). See, for example, Example 10 herein. Protein design typically involves identifying sequences that fold into a given or target structure and can be accomplished using computer models. Computational protein design algorithms search the sequence-conformation space for sequences that are low in energy when folded to the target structure. Computational protein design algorithms use models of protein energetics to evaluate how mutations would affect a protein's structure and function. These energy functions typically include a combination of molecular mechanics, statistical (i.e. knowledge-based), and other empirical terms. Suitable available software includes IPRO (Interactive Protein Redesign and Optimization), EGAD (A Genetic Algorithm for Protein Design), Rosetta Design, Sharpen, and Abalone.

### Linkers

As used herein in the context of polypeptides, the term “linker” or “oligopeptide linker” means one or more amino acids that covalently join two or more functional domains, for example, the MTP and the NP, two NPs, a NP and a tag. The amino acids are covalently joined through peptide bonds, both within the linker and between linker and functional domains. The linker may provide for freedom of movement of one functional domain with respect to the other, without causing a substantial detrimental effect on the function of the two or more domains. The linker may help promote proper folding and functioning of one or both of the functional domains. The skilled person will understand that the size of a linker can be determined empirically or can be modelled based on protein folding information.

The linker may comprise a cleavage site for a protease such as MPP, Such a linker can also be considered to be part of an MTP.

The skilled person will appreciate that the C-terminal end of the MTP can be translationally fused to the N-terminal amino acid of the NP without a linker or via a linker of one or more amino acid residues, for example of 1-5 amino acid residues, Such a linker can also be considered to be part of the MTP.

In embodiments, the linker comprises at least 1 amino acid, at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 12 amino acids, at least 14 amino acids, at least 16 amino acids, at least 18 amino acids, at least 20 amino acids, at least 25 amino acids, at least 30 amino acids, at least 35 amino acids, at least 40 amino acids, the least 45 amino acids, at least 50 amino acids, at least 60 amino acids, at least 70 amino acids, at least 80 amino acids, at least 90 amino acids, or about 100 amino acids. In embodiments, the maximal size of the linker is 100 amino acids, preferably 60 amino acids, more preferably 40 amino acids.

In some embodiments, the linker will permit the movement of one functional domain with respect to the other in order to increase stability of the fusion polypeptide. If desired, the linker can encompass either: repetitions of poly-glycine or combinations of glycine, proline and alanine residues.

Linkers for joining two Nif polypeptides such as NifD-linker-NifK and NifE-linker-NifN are preferably selected, for the number and sequence of the amino acids in the linker, based on several criteria. These are: a lack of cysteine residues to avoid formation of unwanted disulphide linkages, few or preferably no charged residues (Glu, Asp, Arg, Lys) to reduce the likelihood of unwanted surface salt bridge interactions, few or no hydrophobic residues (Phe, Trp, Tyr, Met, Val, Ile, Leu) as such residues may promote a tendency to penetrate the surface of the polypeptide, and lacking amino acids which may be post-translationally modified. In this context “few charged residues” means less than 10% of the amino acid residues in the linker, and “few hydrophobic residues” means less than 15% of the amino acid residues in the linker.

In an embodiment, the linker does not comprise a cysteine residue.

In an embodiment, the linker comprises four, three, or two, or one, or no charged residues. Preferably, in total the linker comprises four, three, or two, or one, or no glutamic acid, asparartic acid, argninine and lysine residues.

In an embodiment, the linker comprises four, three, or two, or one or no hydrophobic residues. Preferably, in total the linker comprises four, three, or two, or one or no phenylalanine, tryptophan, tyrosine, methionine, valine, isoleunce and leucine residues.

In an embodiment, at least 70%, or at least 80%, or at least 90%, of the linker comprises residues selected from threonine, serine, glycine and alanine.

The use of oligopeptide linkers in modifying polypeptides is reviewed in Chen et al. (2013) and Zhang et al. (2009).

### Tags

In a particular embodiment, the fusion polypeptide comprises at least one tag adequate for detection or purification of the fusion polypeptide or a processed product thereof. The tag is typically bound to the C-terminal or N-terminal domain of the fusion polypeptide. In a preferred embodiment, the tag is bound to the C-terminal end of the Nif polypeptide. The tag is generally a peptide or amino acid sequence capable of binding to one or more ligands, for example, one or more ligands of an affinity matrix such as a chromatography support or bead, or an antibody, with high affinity. The skilled person will understand that the tag is preferably located in the fusion protein at a location which does not result in the removal of the tag from the NP once the MTP is cleaved off after import into the mitochondria. Further, the tag should not interfere with the mitochondria import machinery. In a preferred embodiment, the polynucleotide of the invention encodes a fusion polypeptide that comprises, in the N- to C-terminal order, a N-terminal MTP, the Nif polypeptide and the detection/purification tag. In an alternate embodiment, the fusion polypeptide comprises, in the N- to C-terminal order, a N-terminal MTP, the detection/purification tag and the Nif polypeptide.

Additional illustrative, non-limiting examples of tags useful for detecting, isolating or purifying a fusion polypeptide or a processed product thereof include, human influenza hemagglutinin (HA) tag, histidine tags comprising for example, 6 or 8 histidine residues, fluorescent tags such as fluorescein, resourfin and derivatives thereof, Arg-tag, FLAG-tag, Strep-tag, an epitope capable of being recognized by an antibody, such as c-myc-tag (recognized by an anti-c-myc antibody), SBP-tag, S-tag, calmodulin binding peptide, cellulose binding domain, chitin binding domain, glutathione 5-transferase-tag, maltose binding protein, NusA, TrxA, DsbA, Avi-tag, etc.

### Translational Fusions Involving Nif Polypeptides

Translational fusions have been made to several Nif polypeptides as reported in the scientific literature. These are summarised in Table 2 and in the review by Buren and Rubio (2018). Most of them involve the artificial addition of epitopes or binding domains such as Histidine tags or Strep tags to the proteins for detection and purification purposes and only a few have been expressed in plant cells. There are a few reports of naturally occurring fusions between Nif polypeptides, in bacteria. For assays in bacterial hosts, His tags of different lengths (7-10 histidines) were added to NifD (Christiansen et al., 1998), NifE (Goodwin et al., 1998), NifM (Gavini et al., 2006) and both full length and truncated versions of NifB (Fay et al., 2015). In each case, Nif function was retained for the modified Nif polypeptide as demonstrated in bacteria or in in vitro nitrogenase reconstitution assays.

TABLE 2
Summary of gene fusions of Nif polypeptides as reported in the literature

| | | | | |
|---|---|---|---|---|
| | | | Function and | |
| | | Number of | which | |
| | | amino acid | organism (no, | |
| Gene | Naturally or | residues | partial, yes, | |
| fusion | synthetic | changed | not tested) | Reference |

Bacterial expression

| | | | | |
|---|---|---|---|---|
| GST-NafY | synthetic | 26 kDa on N | Yes, in bacteria | Rubio et al., 2004 |
| | | terminus | | |
| NifD-7xHis | synthetic | 7 extra | Yes, in bacteria | Christiansen et |
| at the C | | histidines | | al., 1998 |
| terminus | | | | |
| NifE-NifN | Natural: | Deletion of | Yes, in bacteria | Thiel et al., 1995 |
| | Anabaena | about 10 | | |
| | variabilis | amino acids | | |
| 7xHis-NifE | synthetic | 7 His at N | Yes, in bacteria | Goodwin et al., |
| | | terminus | | 1998 |
| N-(zero | Naturally | | Yes, in bacteria | Dean and |
| linker)-B | occurring in | | | Jackobson, cited |
| | Clostridium | | | in Wiig et al., |
| | pasteruienum | | | 2011 |
| 8xHis-N- | synthetic | Direct NifN- | Yes, in bacteria | Wiig et al., 2011 |
| zero linker- | | NifB fusion, | | |
| B | | zero length | | |
| | | linker | | |
| NifD-NifK | synthetic | Net deletion | partial (50%), | Suh et al., 2003 |
| | | of 3 amino | in bacteria | |
| | | acids, 7 | | |
| | | substitutions | | |
| 7-10xHis- | synthetic | Not specified | Yes, in bacteria | Gavini et al 2006 |
| NifM | | (7-10?) at the | | |
| | | N terminus | | |

Eukaryotic expression

| | | | | |
|---|---|---|---|---|
| His-NifU | synthetic | Epitope at the | Yes, yeast, | Lopez-Torrejon |
| | | N terminus | Cytoplasmic | et al., 2016 |
| | | | location, and | |
| | | | functional if the | |
| | | | yeast are grown | |
| | | | anaerobically | |
| NifB- | Synthetic | 10xHis at C | Yes, tested in | Burén et al., |
| truncated- | | terminus of a | yeast grown | 2017a |
| 10xHis | | NifB | aerobically | |
| | | truncated for | | |
| | | the NifX-like | | |
| | | domain | | |
| MTP-Strep | Synthetic | 28 amino acid | Expressed in | Burén et al., |
| tag-NifB | | N terminal | plants, targeted | 2017a |
| (truncated) | | Strep tag, | to MM, more | |
| | | between the | soluble than the | |
| | | MTP and | full length | |
| | | NifB | version, not | |
| | | | tested for | |
| | | | function | |
| 6xHis- | synthetic | 6 His at N | Yes, tested in | Fay et al., 2015 |
| NifB- | | terminus of a | yeast | PNAS |
| truncated | | NifB | | |
| | | truncated for | | |
| | | the NifX-like | | |
| | | domain at the | | |
| | | C-terminus | | |
| CPN-60- | synthetic | MTP | Yes, in vitro | US2016/0304842 |
| NifS | | targeting in | | |
| | | Nicotiana ssp | | |
| CPN-60- | synthetic | MTP | Yes, in vitro | US2016/0304842 |
| NifU | | targeting in | | |
| | | Nicotiana ssp | | |
| CPN-60- | synthetic | MTP | Yes, in vitro | US2016/0304842 |
| NifH | | targeting in | | |
| | | Nicotiana ssp | | |
| CPN-60- | synthetic | MTP | Yes, in vitro | US2016/0304842 |
| NifM | | targeting in | | |
| | | Nicotiana ssp | | |

Thiel et al. (1995) identified a naturally occurring deletion of 29 nucleotides and therefore deleting 9 amino acids and the NifE stop codon in the intergenic region between the NifE and NUN genes in the blue-green alga Anabaena variabilis. The deletion resulted in a NifE-NifN polypeptide fusion which retained at least some nitrogenase function of the NifE and NifN polypeptides. The NifE-NifN fusion polypeptide also had 19 other amino acid substitutions in the region of the fusion junction, which might have affected Nif function but in unknown ways. The fusion gene was expressed but only under strictly anaerobic conditions. It was not reported if there was a reduction in activity relative to the non-fused genes.

Suh et al. (2003) created an artificial junction between the NifD and NifK genes of the chromosome of A. vinelandii by a deletion including the stop codon of NifD and the translation start codon (ATG) of NifK, forming a vector designated pBG1404. The deletion resulted in a net loss of three amino acids and seven amino acid substitutions in amino acids 2-10 of the NifK polypeptide. The A. vinelandii host cells containing pBG1404 were compromised in their growth in low nitrogen media relative to the corresponding wild-type bacteria.

Wiig at al. (2011) used a naturally occurring translational fusion between NUN and NUB genes found in Clostridium pastuerianum and determined that it is functional for NifN and NifB activity in bacterial and biochemical complementation assays. This fusion was direct without any peptide linker, i.e. the C-terminal end of NifN was directly covalently linked to the N-terminal end of NifB.

In yeast and plant cells, translational fusions have been used to direct proteins encoded in the nucleus to mitochondrial matrix. In yeast expression assays, translational fusions of mitochondrial targeting peptide (MTP) and some Nif polypeptides (NifH, NifM, NifS, and NifU) were shown to be functional when grown under aerobic conditions (Lopez-Torrejon et al., 2016). Epitope fusions (FLAG and HIS) were also shown to be functional when fused to NifH, NifM, NifS and NifU, although these fusions were intended for localisation within the yeast cytoplasm and were only functional when the yeast were grown under anaerobic conditions. Buren et al. (2017b) showed that a mitochondrial-matrix targeted version of a soluble variant of NifB was functional in in vitro complementation assays when re-isolated from the mitochondria of yeast. This version of NifB included a N-terminal MTP, a truncated variant of NifB (without the NifX-like domain) and a C-terminal 10×His epitope tag. A large number of MTP-Nif fusions were also generated in yeast expression assays. However, this large ensemble of co-expressed proteins failed to show activity in yeast (Buren et al., 2017b).

An MTP from a CPN-60 gene was fused to the N-terminal end of NifH, NifM, NifS and NifU and shown to be functional via in vitro complementation assays when the FeProtein was re-isolated from plants grown under reduced oxygen tension at 10% oxygen (US2016/0304842).

### Polynucleotides

The terms “polynucleotide” and “nucleic acid” are used interchangeably herein. They mean a polymeric form of nucleotides of any length, either deoxyribonucleotides or ribonucleotides, or analogs thereof. A polynucleotide defined herein may be of genomic, cDNA, semisynthetic, or synthetic origin, single-stranded or preferably double-stranded and by virtue of its origin or manipulation: (1) is not associated with all or a portion of a polynucleotide with which it is associated in nature (e.g., a Nif polynucleotide that does not comprise a native promoter encoding sequence), (2) is linked to a polynucleotide other than that to which it is linked in nature (e.g., a Nif polynucleotide linked to a MTP encoding nucleotide sequence and/or a non-native promoter encoding sequence), or (3) does not occur in nature (e.g., polynucleotides encoding MTP-Nif fusion polypeptides of the invention). The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), ribozymes, cDNA, recombinant polynucleotides, branched polynucleotides, plasmids, vectors, isolated DNA of any sequence, isolated RNA of any sequence, chimeric DNA of any sequence, nucleic acid probes, and primers. A polynucleotide may comprise modified nucleotides such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization such as by conjugation with a labeling component.

An “isolated polynucleotide” is substantially free from components that are normally linked (e.g., regulatory sequences) or associate with the polynucleotide. Thus, an isolated polynucleotide is substantially free of other cellular material, or culture medium when produced by recombinant techniques, or substantially free of chemical precursors or other chemicals when chemically synthesized. Preferably, the isolated polynucleotide is at least 60% free, more preferably at least 75% free, and more preferably at least 90% free from said components.

As used herein, the phrase “exogenous polynucleotide” refers to a polynucleotide that has a sequence originating from outside the cell or organism that the exogenous polynucleotide is present in.

As used herein, the term “gene” is to be taken in its broadest context and includes the deoxyribonucleotide sequences comprising the transcribed region and, if translated, the protein coding region, of a structural gene and including sequences located adjacent to the coding region on both the 5′ and 3′ ends for a distance of at least about 2 kb on either end and which are involved in expression of the gene. In this regard, the gene includes control signals such as promoters, enhancers, translation and transcription termination and/or polyadenylation signals that are naturally associated with a given gene, or heterologous control signals, in which case, the gene is referred to as a “chimeric gene”. The sequences which are located 5′ of the protein coding region and which are present on the mRNA are referred to as 5′ non-translated sequences. The sequences which are located 3′ or downstream of the protein coding region and which are present on the mRNA are referred to as 3′ non-translated sequences. The term “gene” encompasses both cDNA and genomic forms of a gene. A genomic form or clone of a gene contains the coding region which may be interrupted with non-coding sequences termed “introns”, “intervening regions”, or “intervening sequences.” Introns are segments of a gene which are transcribed into nuclear RNA (nRNA). Introns may contain regulatory elements such as enhancers. Introns are removed or “spliced out” from the nuclear or primary transcript; introns therefore are absent in the mRNA transcript. The mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

As used herein, “chimeric DNA”, also referred to herein as a “DNA construct”, means any DNA molecule that is not naturally found in nature but which artificially joins two DNA parts into a single molecule, each part of which might be found in nature but the whole is not found in nature. For example, a DNA construct encoding a MTP-Nif fusion polypeptide of the invention. Typically, chimeric DNA comprises regulatory and transcribed or protein coding sequences that are not naturally found together in nature (e.g., a Nif polynucleotide linked to a non-native promoter encoding sequence). Accordingly, chimeric DNA may comprise regulatory sequences and coding sequences that are derived from different sources, or regulatory sequences and coding sequences derived from the same source, but arranged in a manner different than that found in nature. The open reading frame may or may not be linked to its natural upstream and downstream regulatory elements. The open reading frame may be incorporated into, for example, the plant genome, in a non-natural location, or in a replicon or vector where it is not naturally found such as a bacterial plasmid or a viral vector. The term “chimeric DNA” is not limited to DNA molecules which are replicable in a host, but includes DNA capable of being ligated into a replicon by, for example, specific adaptor sequences.

A “transgene” is a gene that has been introduced into the genome by a transformation procedure. The term includes a gene in a progeny cell, plant, seed, non-human organism or part thereof which was introducing into the genome of a progenitor cell thereof. Such progeny cells etc may be at least a 3^{rd} or 4^{th} generation progeny from the progenitor cell which was the primary transformed cell. Progeny may be produced by sexual reproduction or vegetatively such as, for example, from tubers in potatoes or ratoons in sugarcane. The term “genetically modified”, and variations thereof, is a broader term that includes introducing a gene into a cell by transformation or transduction, mutating a gene in a cell and genetically altering or modulating the regulation of a gene in a cell, or the progeny of any cell modified as described above.

A “genomic region” as used herein refers to a position within the genome where a transgene, or group of transgenes (also referred to herein as a cluster), have been inserted into a cell, or predecessor thereof. Such regions only comprise nucleotides that have been incorporated by the intervention of man such as by methods described herein.

A “recombinant polynucleotide” of the invention refers to a nucleic acid molecule which has been constructed or modified by artificial recombinant methods. The recombinant polynucleotide may be present in a cell in an altered amount or expressed at an altered rate (e.g., in the case of mRNA) compared to its native state. In one embodiment, the polynucleotide is introduced into a cell that does not naturally comprise the polynucleotide. Typically an exogenous DNA is used as a template for transcription of mRNA which is then translated into a continuous sequence of amino acid residues coding for a polypeptide of the invention within the transformed cell. In another embodiment, the polynucleotide is endogenous to a bacterial cell and its expression is altered by recombinant means, for example, an exogenous control sequence is introduced upstream of an endogenous gene of interest to enable the transformed cell to express the polypeptide encoded by the gene.

A recombinant polynucleotide of the invention includes polynucleotides which have not been separated from other components of the cell-based or cell-free expression system, in which it is present, and polynucleotides produced in said cell-based or cell-free systems which are subsequently purified away from at least some other components. The polynucleotide can be a contiguous stretch of nucleotides existing in nature (e.g., Nil′ polynucleotide), or comprise two or more contiguous stretches of nucleotides from different sources (naturally occurring and/or synthetic) joined to form a single polynucleotide (e.g., a Nif polynucleotide linked to a MTP encoding nucleotide sequence and/or a non-native promoter encoding sequence). Typically, such chimeric polynucleotides comprise at least an open reading frame encoding a polypeptide of the invention operably linked to a promoter suitable of driving transcription of the open reading frame in a cell of interest. Reference to “a promoter” herein encompasses a single promoter or multiple promoters.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

A polynucleotide of, or useful for, the present invention may selectively hybridise, under stringent conditions, to a polynucleotide defined herein. As used herein, stringent conditions are those that: (1) employ during hybridisation a denaturing agent such as formamide, for example, 50% (v/v) formamide with 0.1% (w/v) bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (2) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS, and/or (3) employ low ionic strength and high temperature for washing, for example, 0.015 M NaCl/0.0015 M sodium citrate/0.1% SDS at 50° C.

Polynucleotides of the invention may possess, when compared to naturally occurring molecules, one or more mutations which are deletions, insertions, or substitutions of nucleotide residues. Polynucleotides which have mutations relative to a reference sequence can be either naturally occurring (that is to say, isolated from a natural source) or synthetic (for example, by performing site-directed mutagenesis or DNA shuffling on the nucleic acid as described above).

Polynucleotdies of the invention may be codon-modified for expression in a plant cell. The skilled person will appreciated that the protein coding region may be codon optimised relative to, for example, the coding region of a naturally occurring polynucleotide in a nitrogen fixing bacterium.

### Nucleic Acid Constructs

The present invention includes nucleic acid constructs comprising one or more polynucleotides of the invention, and vectors and host cells containing these, methods of their production and use, and uses thereof. The present invention refers to elements which are operably connected or linked. “Operably connected” or “operably linked” and the like refer to a linkage of polynucleotide elements in a functional relationship. Typically, operably connected nucleic acid sequences are contiguously linked and, where necessary to join two protein coding regions, contiguous and in reading frame. A coding sequence is “operably connected to” another coding sequence when RNA polymerase will transcribe the two coding sequences into a single RNA, which if translated is then translated into a single polypeptide having amino acids derived from both coding sequences. The coding sequences need not be contiguous to one another so long as the expressed sequences are ultimately processed to produce the desired protein.

As used herein, the term “cis-acting sequence”, “cis-acting element” or “cis-regulatory region” or “regulatory region” or similar term shall be taken to mean any sequence of nucleotides, which when positioned appropriately and connected relative to an expressible genetic sequence, is capable of regulating, at least in part, the expression of the genetic sequence. Those skilled in the art will be aware that a cis-regulatory region may be capable of activating, silencing, enhancing, repressing or otherwise altering the level of expression and/or cell-type-specificity and/or developmental specificity of a gene sequence at the transcriptional or post-transcriptional level. In preferred embodiments of the present invention, the cis-acting sequence is an activator sequence that enhances or stimulates the expression of an expressible genetic sequence.

“Operably connecting” a promoter or enhancer element to a transcribable polynucleotide means placing the transcribable polynucleotide (e.g., protein-encoding polynucleotide or other transcript) under the regulatory control of a promoter, which then controls the transcription of that polynucleotide. In the construction of heterologous promoter/structural gene combinations, it is generally preferred to position a promoter or variant thereof at a distance from the transcription start site of the transcribable polynucleotide which is approximately the same as the distance between that promoter and the protein coding region it controls in its natural setting; i.e., the gene from which the promoter is derived. As is known in the art, some variation in this distance can be accommodated without loss of function. Similarly, the preferred positioning of a regulatory sequence element (e.g., an operator, enhancer etc) with respect to a transcribable polynucleotide to be placed under its control is defined by the positioning of the element in its natural setting; i.e., the gene from which it is derived.

“Promoter” or “promoter sequence” as used herein refers to a region of a gene, generally upstream (5′) of the RNA encoding region, which controls the initiation and level of transcription in the cell of interest. A “promoter” includes the transcriptional regulatory sequences of a classical genomic gene, such as a TATA box and CCAAT box sequences, as well as additional regulatory elements (i.e., upstream activating sequences, enhancers and silencers) that alter gene expression in response to developmental and/or environmental stimuli, or in a tissue-specific or cell-type-specific manner. A promoter is usually, but not necessarily (for example, some PolIII promoters), positioned upstream of a structural gene, the expression of which it regulates. Furthermore, the regulatory elements comprising a promoter are usually positioned within 2 kb of the start site of transcription of the gene. Promoters may contain additional specific regulatory elements, located more distal to the start site to further enhance expression in a cell, and/or to alter the timing or inducibility of expression of a structural gene to which it is operably connected.

“Constitutive promoter” refers to a promoter that directs expression of an operably linked transcribed sequence in many or all tissues of an organism such as a plant. The term “constitutive” as used herein does not necessarily indicate that a gene is expressed at the same level in all cell types, but that the gene is expressed in a wide range of cell types, although some variation in level is often detectable. “Selective expression” as used herein refers to expression almost exclusively in specific organs of, for example, the plant, such as, for example, endosperm, embryo, leaves, fruit, tubers or root. In a preferred embodiment, a promoter is expressed selectively or preferentially in roots, leaves and/or stems of a plant, preferably a cereal plant. Selective expression may therefore be contrasted with constitutive expression, which refers to expression in many or all tissues of a plant under most or all of the conditions experienced by the plant.

Selective expression may also result in compartmentation of the products of gene expression in specific plant tissues, organs or developmental stages. Compartmentation in specific subcellular locations such as the plastid, cytosol, vacuole, or apoplastic space may be achieved by the inclusion in the structure of the gene product of appropriate signals, eg. a signal peptide, for transport to the required cellular compartment, or in the case of the semi-autonomous organelles (plastids and mitochondria) by integration of the transgene with appropriate regulatory sequences directly into the organelle genome. A “tissue-specific promoter” or “organ-specific promoter” is a promoter that is preferentially expressed in one tissue or organ relative to many other tissues or organs, preferably most if not all other tissues or organs in, for example, a plant. Typically, the promoter is expressed at a level 10-fold higher in the specific tissue or organ than in other tissues or organs.

In an embodiment, the promoter is a stem-specific promoter, a leaf-specific promoter or a promoter which directs gene expression in an aerial part of the plant (at least stems and leaves) (green tissue specific promoter) such as a ribulose-1,5-bisphosphate carboxylase oxygenase (RUBISCO) promoter.

Examples of stem-specific promoters include, but are not limited to those described in U.S. Pat. No. 5,625,136.

In an embodiment, the promoter is a root specific promoter, Examples of root specific promoters include, but are not limited to, the promoter for the acid chitinase gene and specific subdomains of the CaMV 35S promoter.

The promoters contemplated by the present invention may be native to the host plant to be transformed or may be derived from an alternative source, where the region is functional in the host plant. Other sources include the Agrobacterium T-DNA genes, such as the promoters of genes for the biosynthesis of nopaline, octapine, mannopine, or other opine promoters, tissue specific promoters (see, e.g., U.S. Pat. No. 5,459,252 and WO 91/13992); promoters from viruses (including host specific viruses), or partially or wholly synthetic promoters. Numerous promoters that are functional in mono- and dicotyledonous plants are well known in the art (see, for example, Salomon et al., 1984; Garfinkel et al., 1983; Barker et al., 1983); including various promoters isolated from plants and viruses such as the cauliflower mosaic virus promoter (CaMV 35S, 19S). Non-limiting methods for assessing promoter activity are disclosed by Medberry et al. (1992, 1993), Sambrook et al. (1989, supra) and U.S. Pat. No. 5,164,316.

Alternatively or additionally, the promoter may be an inducible promoter or a developmentally regulated promoter which is capable of driving expression of the introduced polynucleotide at an appropriate developmental stage of the, for example, plant. Other cis-acting sequences which may be employed include transcriptional and/or translational enhancers. Enhancer regions are well known to persons skilled in the art, and can include an ATG translational initiation codon and adjacent sequences. When included, the initiation codon should be in phase with the reading frame of the coding sequence relating to the foreign or exogenous polynucleotide to ensure translation of the entire sequence if it is to be translated. Translational initiation regions may be provided from the source of the transcriptional initiation region, or from a foreign or exogenous polynucleotide. The sequence can also be derived from the source of the promoter selected to drive transcription, and can be specifically modified so as to increase translation of the mRNA.

The nucleic acid construct of the present invention may comprise a 3′ non-translated sequence from about 50 to 1,000 nucleotide base pairs which may include a transcription termination sequence. A 3′ non-translated sequence may contain a transcription termination signal which may or may not include a polyadenylation signal and any other regulatory signals capable of effecting mRNA processing. A polyadenylation signal functions for addition of polyadenylic acid tracts to the 3′ end of a mRNA precursor. Polyadenylation signals are commonly recognized by the presence of homology to the canonical form 5′ AATAAA-3′ although variations are not uncommon. Transcription termination sequences which do not include a polyadenylation signal include terminators for Poll or PolIII RNA polymerase which comprise a run of four or more thymidines. Examples of suitable 3′ non-translated sequences are the 3′ transcribed non-translated regions containing a polyadenylation signal from an octopine synthase (ocs) gene or nopaline synthase (nos) gene of Agrobacterium tumefaciens (Bevan et al., 1983). Suitable 3′ non-translated sequences may also be derived from plant genes such as the ribulose-1,5-bisphosphate carboxylase (ssRUBISCO) gene, although other 3′ elements known to those of skill in the art can also be employed.

As the DNA sequence inserted between the transcription initiation site and the start of the coding sequence, i.e., the untranslated 5′ leader sequence (5′UTR), can influence gene expression if it is translated as well as transcribed, one can also employ a particular leader sequence. Suitable leader sequences include those that comprise sequences selected to direct optimum expression of the foreign or endogenous DNA sequence. For example, such leader sequences include a preferred consensus sequence which can increase or maintain mRNA stability and prevent inappropriate initiation of translation as, for example, described by Joshi (1987).

### Vectors

The present invention includes use of vectors for manipulation or transfer of genetic constructs. A vector is a nucleic acid molecule, preferably a DNA molecule, that can be used to artificially carry foreign genetic material; into another cell, where it can be replicated or expressed. A vector containing foreign DNA is referred to as a “recombinant vector”. Examples of vectors include, but are not limited to, plasmids, viral vectors, cosmids, extrachromosomal elements, minichromosomes, artificial chromosomes. The vector may comprise a transposable element.

A vector preferably is double-stranded DNA and contains one or more unique restriction sites and may be capable of autonomous replication in a defined host cell including a target cell or tissue or a progenitor cell or tissue thereof, or capable of integration into the genome, preferably the nuclear genome, of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into a cell, is integrated into the genome, preferably the nuclear genome, of the recipient cell and replicated together with the chromosome(s) into which it has been integrated. A vector system may comprise a single vector or plasmid, two or more vectors or plasmids, which together contain the total DNA to be introduced into the host cell, or a transposon. The choice of the vector will typically depend on the compatibility of the vector with the cell into which the vector is to be introduced. The vector may also include a selection marker such as an antibiotic resistance gene, a herbicide resistance gene or other gene that can be used for selection of suitable transformants. Examples of such genes are well known to those of skill in the art.

The nucleic acid construct of the invention can be introduced into a vector, such as a plasmid. Plasmid vectors typically include additional nucleic acid sequences that provide for easy selection, amplification, and transformation of the expression cassette in prokaryotic and eukaryotic cells, for example, pUC-derived vectors, pSK-derived vectors, pGEM-derived vectors, pSP-derived vectors, pBS-derived vectors, or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells.

By “marker gene” is meant a gene that imparts a distinct phenotype to cells expressing the marker gene and thus allows such transformed cells to be distinguished from cells that do not have the marker. A selectable marker gene confers a trait for which one can “select” based on resistance to a selective agent (e.g., a herbicide, antibiotic, radiation, heat, or other treatment damaging to untransformed cells). A screenable marker gene (or reporter gene) confers a trait that one can identify through observation or testing, i.e., by “screening” (e.g., β-glucuronidase, luciferase, GFP or other enzyme activity not present in untransformed cells). The marker gene and the nucleotide sequence of interest do not have to be linked.

To facilitate identification of transformants, the nucleic acid construct desirably comprises a selectable or screenable marker gene as, or in addition to, the foreign or exogenous polynucleotide. The actual choice of a marker is not crucial as long as it is functional (i.e., selective) in combination with the host cell, preferably a plant host cell. The marker gene and the foreign or exogenous polynucleotide of interest do not have to be linked, since co-transformation of unlinked genes as, for example, described in U.S. Pat. No. 4,399,216 is also an efficient process in plant transformation.

Examples of bacterial selectable markers are markers that confer antibiotic resistance such as ampicillin, erythromycin, chloramphenicol or tetracycline resistance, preferably kanamycin resistance. Exemplary selectable markers for selection of plant transformants include, but are not limited to, a hyg gene which encodes hygromycin B resistance; a neomycin phosphotransferase (nptII) gene conferring resistance to kanamycin, paromomycin, G418; a glutathione-S-transferase gene from rat liver conferring resistance to glutathione derived herbicides as, for example, described in EP 256223; a glutamine synthetase gene conferring, upon overexpression, resistance to glutamine synthetase inhibitors such as phosphinothricin as, for example, described in WO 87/05327; an acetyltransferase gene from Streptomyces viridochromogenes conferring resistance to the selective agent phosphinothricin as, for example, described in EP 275957; a gene encoding a 5-enolshikimate-3-phosphate synthase (EPSPS) conferring tolerance to N-phosphonomethylglycine as, for example, described by Hinchee et al. (1988); a bar gene conferring resistance against bialaphos as, for example, described in WO91/02071; a nitrilase gene such as bxn from Klebsiella ozaenae which confers resistance to bromoxynil (Stalker et al., 1988); a dihydrofolate reductase (DHFR) gene conferring resistance to methotrexate (Thillet et al., 1988); a mutant acetolactate synthase gene (ALS), which confers resistance to imidazolinone, sulfonylurea or other ALS-inhibiting chemicals (EP 154,204); a mutated anthranilate synthase gene that confers resistance to 5-methyl tryptophan; or a dalapon dehalogenase gene that confers resistance to the herbicide.

Preferred screenable markers include, but are not limited to, a uidA gene encoding a β-glucuronidase (GUS) enzyme for which various chromogenic substrates are known; a β-galactosidase gene encoding an enzyme for which chromogenic substrates are known; an aequorin gene (Prasher et al., 1985), which may be employed in calcium-sensitive bioluminescence detection; a green fluorescent protein gene (Niedz et al., 1995) or derivatives thereof; a luciferase (luc) gene (Ow et al., 1986), which allows for bioluminescence detection, and others known in the art. By “reporter molecule” as used in the present specification is meant a molecule that, by its chemical nature, provides an analytically identifiable signal that facilitates determination of promoter activity by reference to protein product.

Preferably, the nucleic acid construct is stably incorporated into the genome of, for example, the plant. Accordingly, the nucleic acid comprises appropriate elements which allow the molecule to be incorporated into the genome, or the construct is placed in an appropriate vector which can be incorporated into a chromosome of a plant cell.

One embodiment of the present invention includes a recombinant vector, which comprises at least one polynucleotide defined herein, and is capable of delivering the polynucleotide into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

Recombinant vectors of the invention comprise fusion sequences which lead to the expression of nucleic acid molecules as fusion proteins.

Recombinant vectors may also include intervening and/or untranslated sequences surrounding and/or within the nucleic acid sequence of a polynucleotide defined herein.

Preferably, the recombinant vector is stably incorporated into the genome of a host cell such as a plant cell. Accordingly, the recombinant vector may comprise appropriate elements which allow the vector to be incorporated into the genome, or into a chromosome of the cell.

### Recombinant Cells

Another embodiment of the present invention includes a recombinant cell, for example, a recombinant plant cell, which is a host cell transformed with one or more polynucleotides, constructs, or vectors of the present invention, or progeny cells thereof. The term “recombinant cell” is used interchangeably with the term “transgenic cell” herein.

Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed cell in such a manner that their ability to be expressed is retained.

Preferred host cells are plant cells, more preferably cells of a cereal plant, more preferably barley or wheat cells, and even more preferably a wheat cell.

The recombinant cell may be a cell in culture, a cell in vitro, or in an organism such as, for example, a plant, or in an organ such as, for example, a root, leaf or stem. Preferably, the cell is in a plant, more preferably in roots, leaves, and/or stems of a plant.

In an embodiment, expression of active NifDK in a plant cell requires expression of NifD, NifK, NifB, NifE, NifN and optionally, NifU, NifS NifO, NifV, NifY, NifW, and/or NifZ.

In another or further embodiment, expression of active NifH in a plant cell requires expression of NifH and NifM, and optionally, NifU and/or NifN.

In an embodiment, reconstitution of nitrogenase activity in a plant cell requires expression of at least NifD, NifK, NifB, NifE, NifN and NifM.

The skilled person will appreciate that a smaller subset of Nif proteins may result in functional nitrogenase reconstitution in a plant cell. To the best of the inventors' knowledge, the only report of nitrogenase gene transfer to any photosynthetic organism described introduction of NifH in the chloroplast genome of Chlamydomonas (Cheng et al., 2005). NifH was able to complement a chlorophyll biosynthesis mutant, despite the fact that the NifH biosynthetic precursor proteins NifM, NifS and NifU were not co-expressed. This demonstrated that endogenous eukaryotic equivalents may functionally substitute for certain Nif proteins. Indeed a recent report, demonstrating that E. coli can reconstitute nitrogenase function using only eight Nif proteins (Wang et al., 2013), implies achieving function is plants may be less complex than expressing the full complement of Nif proteins. Whilst the inventors have yet to establish functionality of Nif proteins in planta, it is promising that the repertoire of biosynthetic and functional Nif proteins can be expressed in an environment potentially supportive of nitrogenase function.

### Plants

The term “plant” as used herein as a noun refers to whole plants and refers to any member of the Kingdom Plantae, but as used as an adjective refers to any substance which is present in, obtained from, derived from, or related to a plant, such as for example, plant organs (e.g. leaves, stems, roots, flowers), single cells (e.g. pollen), seeds, plant cells and the like. Plantlets and germinated seeds from which roots and shoots have emerged are also included within the meaning of “plant”. The term “plant parts” as used herein refers to one or more plant tissues or organs which are obtained from a plant and which comprises genomic DNA of the plant. Plant parts include vegetative structures (for example, leaves, stems), roots, floral organs/structures, seed (including embryo, cotyledons, and seed coat), plant tissue (for example, vascular tissue, ground tissue, and the like), cells and progeny of the same. In a preferred embodiment, the plant part is a seed. The term “plant cell” as used herein refers to a cell obtained from a plant or in a plant and includes protoplasts or other cells derived from plants, gamete-producing cells, and cells which regenerate into whole plants. Plant cells may be cells in culture. By “plant tissue” is meant differentiated tissue in a plant or obtained from a plant (“explant”) or undifferentiated tissue derived from immature or mature embryos, seeds, roots, shoots, fruits, tubers, pollen, tumor tissue, such as crown galls, and various forms of aggregations of plant cells in culture, such as calli. Exemplary plant tissues in or from seeds are cotyledon, embryo and embryo axis. The invention accordingly includes plants and plant parts and products comprising these.

As used herein, the term “seed” refers to “mature seed” of a plant, which is either ready for harvesting or has been harvested from the plant, such as is typically harvested commercially in the field, or as “developing seed” which occurs in a plant after fertilisation and prior to seed dormancy being established and before harvest.

A “transgenic plant” as used herein refers to a plant that contains a nucleic acid construct not found in a wild-type plant of the same species, variety or cultivar. That is, transgenic plants (transformed plants) contain genetic material (a transgene) that they did not contain prior to the transformation. The transgene may include genetic sequences obtained from or derived from a plant cell, or another plant cell, or a non-plant source, or a synthetic sequence. Typically, the transgene has been introduced into the plant by human manipulation such as, for example, by transformation but any method can be used as one of skill in the art recognizes. The genetic material is preferably stably integrated into the genome of the plant, preferably the nuclear genome. The introduced genetic material may comprise sequences that naturally occur in the same species but in a rearranged order or in a different arrangement of elements, for example an antisense sequence. Plants containing such sequences are included herein in “transgenic plants”.

In a preferred embodiment, the transgenic plants are homozygous for each and every gene that has been introduced (transgene) so that their progeny do not segregate for the desired phenotype. The transgenic plants may also be heterozygous for the introduced transgene(s), such as, for example, in F1 progeny which have been grown from hybrid seed. Such plants may provide advantages such as hybrid vigour, well known in the art.

Transgenic plants, as defined in the context of the present invention include progeny of the plants which have been genetically modified using recombinant techniques, wherein the progeny comprise the transgene of interest. Such progeny may be obtained by self-fertilisation of the primary transgenic plant or by crossing such plants with another plant of the same species. This would generally be to modulate the production of at least one protein defined herein in the desired plant or plant organ. Transgenic plant parts include all parts and cells of said plants comprising the transgene such as, for example, cultured tissues, callus and protoplasts.

Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

A “non-transgenic plant” is one which has not been genetically modified by the introduction of genetic material by recombinant DNA techniques. As used herein, the term “compared to an isogenic plant”, or similar phrases, refers to a plant which is isogenic relative to the transgenic plant but without the transgene of interest. Preferably, the corresponding non-transgenic plant is of the same cultivar or variety as the progenitor of the transgenic plant of interest, or a sibling plant line which lacks the construct, often termed a “segregant”, or a plant of the same cultivar or variety transformed with an “empty vector” construct, and may be a non-transgenic plant. “Wild type”, as used herein, refers to a cell, tissue or plant that has not been modified according to the invention. Wild-type cells, tissue or plants may be used as controls to compare levels of expression of an exogenous nucleic acid or the extent and nature of trait modification with cells, tissue or plants modified as described herein.

Transgenic plants, as defined in the context of the present invention include progeny of the plants which have been genetically modified using recombinant techniques, wherein the progeny comprise the transgene of interest. Such progeny may be obtained by self-fertilisation of the primary transgenic plant or by crossing such plants with another plant of the same species. Transgenic plant parts include all parts and cells of said plants comprising the transgene such as, for example, cultured tissues, callus and protoplasts.

Plants contemplated for use in the practice of the present invention include both monocotyledons and dicotyledons. Target plants include, but are not limited to, the following: cereals (for example, wheat, barley, rye, oats, rice, maize, sorghum and related crops); grapes; beet (sugar beet and fodder beet); pomes, stone fruit and soft fruit (apples, pears, plums, peaches, almonds, cherries, strawberries, raspberries and black-berries); leguminous plants (beans, lentils, peas, soybeans); oil plants (rape or other Brassicas, mustard, poppy, olives, sunflowers, safflower, flax, coconut, castor oil plants, cocoa beans, groundnuts); cucumber plants (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf, bananas and natural rubber plants, as well as ornamentals (flowers, shrubs, broad-leaved trees and evergreens, such as conifers). Preferably, the plant is a cereal plant, more preferably wheat, rice, maize, triticale, oats or barley, even more preferably wheat.

As used herein, the term “wheat” refers to any species of the Genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. macha, T. compactum, T. sphaerococcum, T. vavilovii, and interspecies cross thereof. A preferred species of hexaploid wheat is T. aestivum ssp aestivum (also termed “breadwheat”). Tetraploid wheat includes T. durum (also referred to herein as durum wheat or Triticum turgidum ssp. durum), T. dicoccoides, T. dicoccum, T. polonicum, and interspecies cross thereof. In addition, the term “wheat” includes potential progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. Particularly preferred progenitors are those of the A genome, even more preferably the A genome progenitor is T. monococcum. A wheat cultivar for use in the present invention may belong to, but is not limited to, any of the above-listed species. Also encompassed are plants that are produced by conventional techniques using Triticum sp. as a parent in a sexual cross with a non-Triticum species (such as rye [Secale cereale]), including but not limited to Triticale.

As used herein, the term “barley” refers to any species of the Genus Hordeum, including progenitors thereof, as well as progeny thereof produced by crosses with other species. It is preferred that the plant is of a Hordeum species which is commercially cultivated such as, for example, a strain or cultivar or variety of Hordeum vulgare or suitable for commercial production of grain.

### Methods for Producing Transgenic Plants

Four general methods for direct delivery of a gene into cells have been described: (1) chemical methods (Graham et al., 1973); (2) physical methods such as microinjection (Capecchi, 1980); electroporation (see, for example, WO 87/06614, U.S. Pat. Nos. 5,472,869, 5,384,253, WO 92/09696 and WO 93/21335); and the gene gun (see, for example, U.S. Pat. Nos. 4,945,050 and 5,141,131); (3) viral vectors (Clapp, 1993; Lu et al., 1993; Eglitis et al., 1988); and (4) receptor-mediated mechanisms (Curiel et al., 1992; Wagner et al., 1992).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required. A particle delivery system suitable for use with the present invention is the helium acceleration PDS-1000/He gun is available from Bio-Rad Laboratories. For the bombardment, immature embryos or derived target cells such as scutella or calli from immature embryos may be arranged on solid culture medium.

In another alternative embodiment, plastids can be stably transformed. Method disclosed for plastid transformation in higher plants include particle gun delivery of DNA containing a selectable marker and targeting of the DNA to the plastid genome through homologous recombination (U.S. Pat. Nos. 5,451,513, 5,545,818, 5,877,402, 5,932,479, and WO 99/05265.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. Nos. 5,177,010, 5,104,310, 5,004,863, 5,159,135). Further, the integration of the T-DNA is a relatively precise process resulting in few rearrangements. The region of DNA to be transferred is defined by the border sequences, and intervening DNA is usually inserted into the plant genome.

Agrobacterium transformation vectors are capable of replication in E. coli as well as Agrobacterium, allowing for convenient manipulations as described (Klee et al., Plant DNA Infectious Agents, Hohn and Schell, (editors), Springer-Verlag, New York, (1985): 179-203). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

A transgenic plant formed using Agrobacterium transformation methods typically contains a single genetic locus on one chromosome. Such transgenic plants can be referred to as being hemizygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants for the gene of interest.

It is also to be understood that two different transgenic plants can also be mated to produce offspring that contain two independently segregating exogenous genes. Selfing of appropriate progeny can produce plants that are homozygous for both exogenous genes. Back-crossing to a parental plant and out-crossing with a non-transgenic plant are also contemplated, as is vegetative propagation. Descriptions of other breeding methods that are commonly used for different traits and crops can be found in Fehr, Breeding Methods for Cultivar Development, J. Wilcox (editor) American Society of Agronomy, Madison Wis. (1987).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., Methods for Plant Molecular Biology, Academic Press, San Diego, (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired exogenous nucleic acid is cultivated using methods well known to one skilled in the art.

Methods for transforming dicots, primarily by use of Agrobacterium tumefaciens, and obtaining transgenic plants have been published for cotton (U.S. Pat. Nos. 5,004,863, 5,159,135, 5,518,908); soybean (U.S. Pat. Nos. 5,569,834, 5,416,011); Brassica (U.S. Pat. No. 5,463,174); peanut (Cheng et al., 1996); and pea (Grant et al., 1995).

Methods for transformation of cereal plants such as wheat and barley for introducing genetic variation into the plant by introduction of an exogenous nucleic acid and for regeneration of plants from protoplasts or immature plant embryos are well known in the art, see for example, CA 2,092,588, AU 61781/94, AU 667939, U.S. Pat. No. 6,100,447, WO 97/048814, U.S. Pat. Nos. 5,589,617, 6,541,257, and other methods are set out in WO 99/14314. Preferably, transgenic wheat or barley plants are produced by Agrobacterium tumefaciens mediated transformation procedures. Vectors carrying the desired nucleic acid construct may be introduced into regenerable wheat cells of tissue cultured plants or explants, or suitable plant systems such as protoplasts. The regenerable wheat cells are preferably from the scutellum of immature embryos, mature embryos, callus derived from these, or the meristematic tissue.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (M. J. McPherson and S. G Moller (editors), BIOS Scientific Publishers Ltd, Oxford, (2000)). PCR can be performed on cDNA obtained from reverse transcribing mRNA isolated from plant cells expressing a polynucleotide of the invention. However, it will generally be easier if PCR is performed on genomic DNA isolated from a plant.

A primer is an oligonucleotide sequence that is capable of hybridising in a sequence specific fashion to the target sequence and being extended during the PCR. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the introduction of restriction enzyme or catalytic nucleic acid recognition/cleavage sites in specific target sequences. Primers may also contain additional sequences and/or contain modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al. (supra) and Sambrook et al. (supra). Sequencing can be carried out by any suitable method, for example, dideoxy sequencing, chemical sequencing or variations thereof. Direct sequencing has the advantage of determining variation in any base pair of a particular sequence.

### Plant/Grain Processing

Grain/seed of the invention, preferably cereal grain, or other plant parts of the invention, can be processed to produce a food ingredient, food or non-food product using any technique known in the art.

In one embodiment, the product is whole grain flour such as, for example, an ultrafine-milled whole grain flour, or a flour made from about 100% of the grain. The whole grain flour includes a refined flour constituent (refined flour or refined flour) and a coarse fraction (an ultrafine-milled coarse fraction).

Refined flour may be flour which is prepared, for example, by grinding and bolting cleaned grain such as wheat or barley grain. The particle size of refined flour is described as flour in which not less than 98% passes through a cloth having openings not larger than those of woven wire cloth designated “212 micrometers (U.S. Wire 70)”. The coarse fraction includes at least one of: bran and germ. For instance, the germ is an embryonic plant found within the grain kernel. The germ includes lipids, fiber, vitamins, protein, minerals and phytonutrients, such as flavonoids. The bran includes several cell layers and has a significant amount of lipids, fiber, vitamins, protein, minerals and phytonutrients, such as flavonoids. Further, the coarse fraction may include an aleurone layer which also includes lipids, fiber, vitamins, protein, minerals and phytonutrients, such as flavonoids. The aleurone layer, while technically considered part of the endosperm, exhibits many of the same characteristics as the bran and therefore is typically removed with the bran and germ during the milling process. The aleurone layer contains proteins, vitamins and phytonutrients, such as ferulic acid.

Further, the coarse fraction may be blended with the refined flour constituent. The coarse fraction may be mixed with the refined flour constituent to form the whole grain flour, thus providing a whole grain flour with increased nutritional value, fiber content, and antioxidant capacity as compared to refined flour. For example, the coarse fraction or whole grain flour may be used in various amounts to replace refined or whole grain flour in baked goods, snack products, and food products. The whole grain flour of the present invention (i.e.-ultrafine-milled whole grain flour) may also be marketed directly to consumers for use in their homemade baked products. In an exemplary embodiment, a granulation profile of the whole grain flour is such that 98% of particles by weight of the whole grain flour are less than 212 micrometers.

In further embodiments, enzymes found within the bran and germ of the whole grain flour and/or coarse fraction are inactivated in order to stabilize the whole grain flour and/or coarse fraction. Stabilization is a process that uses steam, heat, radiation, or other treatments to inactivate the enzymes found in the bran and germ layer. Flour that has been stabilized retains its cooking characteristics and has a longer shelf life.

In additional embodiments, the whole grain flour, the coarse fraction, or the refined flour may be a component (ingredient) of a food product and may be used to product a food product. For example, the food product may be a bagel, a biscuit, a bread, a bun, a croissant, a dumpling, an English muffin, a muffin, a pita bread, a quickbread, a refrigerated/frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut, an egg roll, an extruded snack, a fruit and grain bar, a microwaveable snack product, a nutritional bar, a pancake, a par-baked bakery product, a pretzel, a pudding, a granola-based product, a snack chip, a snack food, a snack mix, a waffle, a pizza crust, animal food or pet food.

In alternative embodiments, the whole grain flour, refined flour, or coarse fraction may be a component of a nutritional supplement. For instance, the nutritional supplement may be a product that is added to the diet containing one or more additional ingredients, typically including: vitamins, minerals, herbs, amino acids, enzymes, antioxidants, herbs, spices, probiotics, extracts, prebiotics and fiber. The whole grain flour, refined flour or coarse fraction of the present invention includes vitamins, minerals, amino acids, enzymes, and fiber. For instance, the coarse fraction contains a concentrated amount of dietary fiber as well as other essential nutrients, such as B-vitamins, selenium, chromium, manganese, magnesium, and antioxidants, which are essential for a healthy diet. For example 22 grams of the coarse fraction of the present invention delivers 33% of an individual's daily recommend consumption of fiber. The nutritional supplement may include any known nutritional ingredients that will aid in the overall health of an individual, examples include but are not limited to vitamins, minerals, other fiber components, fatty acids, antioxidants, amino acids, peptides, proteins, lutein, ribose, omega-3 fatty acids, and/or other nutritional ingredients. The supplement may be delivered in, but is not limited to the following forms: instant beverage mixes, ready-to-drink beverages, nutritional bars, wafers, cookies, crackers, gel shots, capsules, chews, chewable tablets, and pills. One embodiment delivers the fiber supplement in the form of a flavored shake or malt type beverage, this embodiment may be particularly attractive as a fiber supplement for children.

In an additional embodiment, a milling process may be used to make a multi-grain flour or a multi-grain coarse fraction. For example, bran and germ from one type of grain may be ground and blended with ground endosperm or whole grain cereal flour of another type of cereal. Alternatively bran and germ of one type of grain may be ground and blended with ground endosperm or whole grain flour of another type of grain. It is contemplated that the present invention encompasses mixing any combination of one or more of bran, germ, endosperm, and whole grain flour of one or more grains. This multi-grain approach may be used to make custom flour and capitalize on the qualities and nutritional contents of multiple types of cereal grains to make one flour.

It is contemplated that the whole grain flour, coarse fraction and/or grain products of the present invention may be produced by any milling process known in the art. An exemplary embodiment involves grinding grain in a single stream without separating endosperm, bran, and germ of the grain into separate streams. Clean and tempered grain is conveyed to a first passage grinder, such as a hammermill, roller mill, pin mill, impact mill, disc mill, air attrition mill, gap mill, or the like. After grinding, the grain is discharged and conveyed to a sifter. Further, it is contemplated that the whole grain flour, coarse fraction and/or grain products of the present invention may be modified or enhanced by way of numerous other processes such as: fermentation, instantizing, extrusion, encapsulation, toasting, roasting, or the like.

### Malting

A malt-based beverage provided by the present invention involves alcohol beverages (including distilled beverages) and non-alcohol beverages that are produced by using malt as a part or whole of their starting material. Examples include beer, happoshu (low-malt beer beverage), whisky, low-alcohol malt-based beverages (e.g., malt-based beverages containing less than 1% of alcohols), and non-alcohol beverages.

Malting is a process of controlled steeping and germination followed by drying of the grain such as barley and wheat grain. This sequence of events is important for the synthesis of numerous enzymes that cause grain modification, a process that principally depolymerizes the dead endosperm cell walls and mobilizes the grain nutrients. In the subsequent drying process, flavour and colour are produced due to chemical browning reactions. Although the primary use of malt is for beverage production, it can also be utilized in other industrial processes, for example as an enzyme source in the baking industry, or as a flavouring and colouring agent in the food industry, for example as malt or as a malt flour, or indirectly as a malt syrup, etc.

In one embodiment, the present invention relates to methods of producing a malt composition. The method preferably comprises the steps of:
(i) providing grain, such as barley or wheat grain, of the invention,
(ii) steeping said grain,
(iii) germinating the steeped grains under predetermined conditions and
(iv) drying said germinated grains.

For example, the malt may be produced by any of the methods described in Hoseney (Principles of Cereal Science and Technology, Second Edition, 1994: American Association of Cereal Chemists, St. Paul, Minn.). However, any other suitable method for producing malt may also be used with the present invention, such as methods for production of speciality malts, including, but limited to, methods of roasting the malt.

Malt is mainly used for brewing beer, but also for the production of distilled spirits. Brewing comprises wort production, main and secondary fermentations and post-treatment. First the malt is milled, stirred into water and heated. During this “mashing”, the enzymes activated in the malting degrade the starch of the kernel into fermentable sugars. The produced wort is clarified, yeast is added, the mixture is fermented and a post-treatment is performed.

### Detection of Nitrogenase Complex

Detection of the nitrogenase complex can be carried out by any method which allows for the detection of the interaction between the NifDK protein complex and the NifH protein. Methods suitable for detecting the interaction between the NifDK protein complex and the NifH protein include any method known in the art for detecting protein-protein interaction including co-immunoprecipitation, affinity blotting, pull down, FRET and the like.

Alternatively, the detection of the nitrogenase complex can be carried out by measuring the activity of the resulting nitrogenase complex.

Methods suitable for measuring nitrogenase activity include any method known in the art for detecting the enzymatic reduction of dinitrogen to ammonia wherein electrons are transferred from the NifH protein to the NifDK protein complex. For example, the nitrogen fixation activity can be estimated by the acetylene reduction assay. Briefly, this technique is an indirect method which uses the ability of the nitrogenase complex to reduce triple bounded substrates. The nitrogenase enzyme reduces acetylene (C2H2) to ethylene (C2H4). Both gases can be quantified using gas chromatography. Nitrogen fixation may also be measured by the hydrogen evolution assay. H2 is an obligate by-product of N₂ fixation. An indirect measure of nitrogenase activity can thereofere be obtained by quantifying the H2 concentration in a gas stream using a flow-through H2 sensor or gas chromatograph.

### Detection of N₂ Fixation

Nitrogen fixation can be estimated by determining a net increase in total N of a plant-soil system (N balance method); 2) separating plant N into the fraction taken up from the soil and the fraction derived from the N₂ fixation (N difference, 15N natural abundance, 15N isotype dilution and ureide methods) and 3) measuring the activity of the nitroegnase (acetylene reduction and hydrogen evolution assays).

### Examples

### Example 1. Materials and Methods

### Expression of Genes in Plant Cells in a Transient Expression System

Genes were expressed in plant cells using a transient expression system essentially as described by Wood et al. (2009), with various modifications as outlined below. Nicotiana benthamiana plants were grown in a growth chamber at 23° C. under a 16:8 h light:dark cycle with 90 mmol/min light intensity provided by cool white fluorescent lamps. Binary vectors containing the coding region to be expressed in plant cells by a strong, constitutive 35S promoter or the enhanced 35S promoter (e35S; Kay et al., 1987) were introduced into Agrobacterium tumefaciens strain GV3101. A chimeric binary vector, 35S::p19, for expression of the p19 viral silencing suppressor was separately introduced into A. tumefaciens strain AGL1, as described in WO2010/057246. This viral silencing suppressor was routinely included in the method to maintain gene expression of transgenes introduced together with it. The recombinant A. tumefaciens cells were grown to stationary phase at 28° C. in LB broth supplemented with 50 mg/L carbenicillin or 50 mg/L kanamycin, according to the selectable marker gene on the vector, and 50 mg/L rifampicin. Acetosyringone was added to the culture to a final concentration of 100 μM and the culture then incubated at 28° C. with shaking for another 2.5 hr. The bacteria were then pelleted by centrifugation at 5000×g for 10 min at room temperature. The supernatant was discarded and the pellet was resuspended in a solution containing 10 mM MES pH 5.7, 10 mM MgCl₂ and 100 μM acetosyringone after which the OD600 was measured. A volume of each culture, including the culture containing the viral suppressor construct 35S::p19, required to reach a final concentration of OD600=0.10 was added to a fresh tube. The final volume was made up with the infiltration buffer. Leaves were then infiltrated with the culture mixture and the plants were typically grown for a further three to five days after infiltration before leaf discs were recovered for analysis. A control infiltration was typically included which had only the viral suppressor construct 35S::p19.

For over-expression of more than one gene of interest in combination, each additional gene was introduced separately into an A. tumefaciens strain and grown as described above. Bacterial suspensions were mixed so that each bacterial strain was at a final concentration of OD600=0.10. The bacterial strain containing the gene encoding the viral silencing suppressor 35S::p19 was included in all mixtures at the same concentration. For example, to express four genes in a transient leaf assay and including the viral suppressor construct, the final OD600 of the infiltrated mixture was 5×0.10=0.50 units. The simultaneous over-expression of at least five genes each from separate T-DNA vectors within plant cells in the transient assay format has previously been demonstrated using Nicotiana benthamiana (Wood et al., 2009).

Construction of Plasmids for Expression of Nif Genes in N. benthamiana Leaves

Unless otherwise stated, plasmids for transient expression of genes in N. benthamiana leaves were constructed using a modular cloning system with Golden Gate assembly (Weber et al., 2011). DNA parts as individual plasmids (Thermo Fisher Scientific, ENSA), each containing the 35S CaMV promoter (EC51288), the gene coding for the first 51 amino acids of the Arabidopsis thaliana F1-ATPase γ subunit (MTP-FAγ51), plant codon-optimised nifH (EC38011), nifK (EC38015), nifY (EC38019), nifE (EC38016), nifN (EC38024), nifJ (EC38022), nifB (EC38017), nifQ (EC38025), nifF (EC38021), nifU (EC38026), nifS (EC38018), nifV (EC38020), nifW (EC38027), nifZ (EC38029), nifM (EC38023), nifX (EC38028), plant codon optimised HA epitope tag (EC38003), and a CaMV polyadenylation sequence/transcription terminator region (EC41414) were assembled into backbone vectors (EC47772, EC47742, EC47751, EC47761, EC47781) using Type IIS restriction cloning.

RNA Extraction, cDNA Synthesis and Analysis

In order to extract RNA from plant leaf samples such as those which have been infiltrated with Agrobacterium, leaf pieces of about 2×2 cm in area are frozen with liquid nitrogen, ground to a powder, and 500 μl of Trizol buffer (Thermo Fisher Scientific) added per sample. Following this, the Trizol supplier's instructions are followed except with these modifications: the chloroform extraction is repeated and the RNA is dissolved at 37° C. The extracted RNA is treated with RQ1 DNAse (Promega) to remove any extracted DNA. The RNA preparations are then further purified using Plant RNeasy columns (Qiagen). When performed, cDNA synthesis is carried out using Superscript III reverse transcriptase (Thermo Fisher Scientific) according to the supplier's protocol with an oligo-dT primer. For RT-PCR analysis of each RNA sample, three separate cDNA synthesis reactions are carried out. The 20 μl cDNA reactions are diluted 20-fold in nuclease free water. qRT-PCR is carried out on a Qiagen rotor gene Q real-time PCR machine. 9.6 μl of each cDNA is added to 10 μl of 2× sensifast no ROX SYBR Taq (Bioline) and 0.4 μl of forward and reverse primers at 10 μmol each, for a final reaction volume of 20 μl. All qPCR reactions (for both reference and specific genes) are carried out in triplicate under the following cycling conditions: 1 cycle of 95° C./5 min, 45 cycles of 95° C./15 sec, 60° C./15 sec and 72° C./20 sec. Fluorescence is measured at the 72° C. step. A 55° C. to 99° C. melting cycle is then carried out. Control amplifications for a constitutively expressed N. benthamiana GADPH mRNA are used to normalise gene expression using the comparative quantitation program in the rotor gene software package. The values for each set of three cDNAs, representing the average of triplicate assays, are averaged, allowing for a calculation of the standard error of the mean (SEM).

Protein Extraction from Bacterial Cells

Protein was isolated from E. coli cells by extraction with Urea/SDS buffer (8 M Urea, 2% SDS, 100 mM Tris-HCl pH 8.5, 65 mM DTT). 300 μl of extraction buffer was added and the mixture vortexed for 10 sec and centrifuged at 12,000×g for 2 min. Supernatants containing the extracted proteins (“total proteins”) were stored at ˜80° C. prior to processing. Protein estimations were performed using the microtiter Bradford protein assay (Bio-Rad, California, USA) according to the manufacturer's instructions. For this, extracted proteins from different samples were diluted in water over two dilutions (1:20, 1:40) in duplicate and measurements were made at 595 nm using a SpectraMax Plus. Bovine serum albumin (BSA) standard was used in the linear range 0.05 mg/mL to approximately 0.5 mg/mL. The BSA concentration was determined by high sensitivity amino acid analysis at the Australian Proteomics Analysis Facility (Sydney, Australia). Blank-corrected standard curves were run in duplicate. Linear regression was used to fit the standard curve.

Protein Extraction from Leaf Tissue

To analyse the amount and properties of specific polypeptides produced in plant cells after T-DNA introduction, especially the size of the polypeptides as an indicator of processing in mitochondria, N. benthamiana leaf samples were harvested by excising about 180 mm 2 leaf pieces from the infiltrated regions 4 or 5 days after infiltration, unless otherwise stated. These were frozen in liquid nitrogen and, when to be processed, were ground to a powder using a mortar and pestle. 300 μL, of buffer was added to each powder sample. The buffer contained 125 mM Tris-HCl pH 6.8, 4% (w/v) sodium dodecyl sulphate (SDS), 20% (w/v) glycerol, 60 mM dithiothreitol (DTT) and 0.002% (w/v) bromophenol blue. Samples were heated at 95° C. for 3 min before centrifugation at 12000×g for 2 min. Supernatant containing the extracted polypeptides, referred to herein as “total protein” samples, was removed and 10 μL to 100 μL, used for Western blotting depending on the expected level of polypeptide to be detected.

Preparation of Total, Insoluble and Soluble Protein Fractions from Leaf Tissue

N. benthamiana leaf samples were harvested by excising about 180 mm 2 leaf pieces from the infiltrated regions 4 or 5 days after infiltration. These were frozen in liquid nitrogen and, when to be processed, were ground to a powder using a mortar and pestle.

For solubility testing the harvested leaf tissue was ground in liquid nitrogen and transferred to a microfuge tube containing extraction buffer (100 mM Tris pH 8.0, 150 mM NaCl, 0.25 M mannitol, 5% (v/v) glycerol, 1% (v/v) Tween 20, 1% (w/v) PVP, 2 mM TCEP, 0.2 mM PMSF, 10 μM leupeptin). The sample was centrifuged at 20,000×g for 5 min to divide the sample into soluble (supernatant) and insoluble (pellet) fractions. The supernatant was transferred to a fresh microfuge tube and centrifuged again at 20,000×g for 5 min, and the pellet was washed three times with extraction buffer. Laemmli buffer was added to the resulting soluble and insoluble fractions and subjected to SDS-PAGE followed by Western blot analysis as described in Allen et al. (2017).

300 μL of cold solubility buffer was added to each ground sample. The solubility buffer contained 50 mM Tris-HCl pH 8.0, 75 mM NaCl, 100 mM mannitol, 2 mM DTT, 0.5% (w/v) polyvinylpyrrolidone (average mol wt 40,000), 5% (v/v) glycerol, 0.2 mM PMSF, 10 μM leupeptin and 0.5% (v/v) Tween® 20. The samples were centrifuged for min at 16,000×g at 4° C. The supernatant was transferred to a fresh tube and the pellet was resuspended in 300 μL of cold solubility buffer. Both, the supernatant (sample 1) and the resuspended pellet (sample 2) were centrifuged again for 5 min at 16,000×g at 4° C. From sample 1, a sample was taken from the supernatant, which is referred to as the soluble fraction. This sample was mixed with an equivalent amount of 4×SDS buffer. 4×SDS buffer contained 250 mM Tris-HCl pH 6.8, 8% (w/v) SDS, 40% (v/v) glycerol, 120 mM DTT and 0.004% (w/v) bromophenol blue. After the second centrifugation step, the supernatant of sample 2 was discarded. The pellet is referred to as the insoluble fraction. The pellet was resuspended in 300 μL 4×SDS buffer and 300 μL of solubility buffer were added. When soluble and insoluble fractions were compared to the amount of total protein, the leaf piece for the total protein sample was ground as described above. However, the ground sample was resuspended in 300 μL 4×SDS buffer and 300 μL of solubility buffer were added. Samples for the total, insoluble and soluble fractions were heated at 95° C. for 3 min and then centrifuged at 12000×g for 2 min. 20 μL of the supernatant containing the extracted polypeptides was loaded on a NuPAGE Bis Tris 4-12% gels (Thermo Fisher Scientific) for gel electrophoresis and Western blot analysis.

For Western blot analysis of anaerobically extracted proteins, the extractions were carried out in an anaerobic chamber (COY Laboratory Products) filled with a H2/N₂ atmosphere (2-3%/97-98%). Anaerobic extraction solutions were prepared at a Schlenk line in a bottle equipped with a butyl rubber septum by at least four cycles of evacuating and purging with N₂.

Purification from Plants of Polypeptides Fused to a TwinStrep Epitope

N. benthamiana leaf samples were harvested five days after infiltration with Agrobacterium containing the genetic construct of interest, or from stably transformed plant leaves, and treated as follows. Leaf material of 15-20 g was macerated in 100 ml cold extraction buffer under anaerobic conditions (<5 ppm O₂) using a stick blender with 6× five second pulses, keeping the mixture cold on ice throughout. The homogenised mixture was filtered through four layers of mira cloth and the filtrate (70-80 ml) centrifuged for 30 min at 3800 g at 4° C. The supernatant was decanted and filtered through a 0.45 μM filter PVDF membrane to further remove fine particulates. The filtrate (60-70 ml) was loaded onto a StreptactinXT column (2 mL bed volume) at 2 mL/min. The column was washed with 20 mL wash buffer before eluting the polypeptides containing the TS epitope using buffer containing 50 mM biotin, 50 mM Tris pH 8.0 and 75 mM NaCl (Elution buffer). The collected fraction numbers 2-8 of 3 mL each were further concentrated over a 10 kDa molecular weight cut-off membrane (10Kda MWCO, Amersham) by centrifugation for 30 min at 3800×g. The purified protein concentrate was snap frozen in liquid nitrogen for future analysis. Samples were retained from each step of the purification process for Western blot analysis conducted at normal atmosphere. Samples and molecular weight markers (BenchMark ladder) were electrophoresed on 4-20% NuPage gels for 60 minutes at 200V, using 20 μL of sample per lane. Proteins in the gels were blotted to PVDF membrane using an iBLOT apparatus and proteins containing an epitope detected by using anti-HA (1:10000) and anti-STREP:HRP (1-step) antibodies.

### Western Blot Analysis

Polypeptides in extracted samples were separated by SDS-polyacrylamide gel electrophoresis (SDS-PAGE) on NuPAGE Bis Tris 4-12% gels (Thermo Fisher Scientific) at 200 V for about 1 hr. The separated polypeptides were transferred from each gel to a PVDF membrane using a dry apparatus (iBLOT) according to the supplier's instructions (Thermo Fisher Scientific) using a three-step 7 min transfer program (1 min at 20 V, 4 min at 23 V and 2 min at 25 V. After blotting, the gels were retained and stained with Coomassie stain (SimplyBlue SafeStain, Thermo Fisher Scientific) overnight, then rinsed in water for visualisation of remaining proteins to confirm that transfer of the polypeptides had occurred. The staining with Coomassie stain also provided confirmation of the equal loading of protein amounts per gel lane, using the levels of highly abundant proteins such as Rubisco large and small subunits as an indicator of equal protein loading per lane. Membranes with bound polypeptides were blocked overnight in TBST buffer containing 5% skim milk powder at 4° C. TBST buffer contained 50 mM Tris-HCl, pH 7.5, 150 mM NaCl and 0.1% (v/v) Tween® 20. Monoclonal anti-HA antibody produced in mouse and anti-rabbit IgG (whole molecule)-peroxidase antibody produced in goat were purchased from Sigma-Aldrich Immun-Star Goat Anti-Mouse (GAM)-HRP conjugate was purchased from Bio-Rad. Anti-isocitrate dehydrogenase (IDH) antibody produced in rabbit was purchased from Agrisera. StrepMABclassic-HRP conjugate antibody was purchased from IBA. Anti-GFP antibody was a gift from Leila Blackman (Australian National University, Canberra, Australia). Anti-HA, anti-IDH and anti-GFP antibodies were added at a 1:5000 dilution, StrepMABclassic-HRP conjugate antibody was added at a 1:10000 dilution in TBST with 5% skim milk powder and the membranes were incubated in the solution for 1 to 2 h. Membranes were then washed for 3×20 min with TBST. When the StrepMABclassic-HRP conjugate antibody was used, the antibodies were detected at this stage using the Amersham ECL reagent (GE Healthcare) and membranes were developed on an Amersham imager 600 (GE Healthcare). For anti-HA and anti-GFP, the secondary antibody anti-Mouse-HRP conjugate was added at 1:5000 in TBST containing 5% skim milk and the membranes incubated for 1 h. For anti-IDH, the secondary antibody anti-rabbit-peroxidase was added at a dilution of 1:5000 in TBST containing 5% skim milk and the membranes were incubated for 1 hour. The membranes were washed for 3×15 min with TBST. For secondary antibody detection, Amersham ECL reagent was used and membranes were developed on an Amersham imager.

Treatment of Protein Extracts with Trypsin Prior to LC-MS Analyses

When used for LC-MS analysis, protein samples were subjected to filter-aided sample preparation (FASP), a method used for the on-filter digestion of proteins prior to mass-spectrometry-based analyses (Wisniewski et al., 2011). In brief, 100 μl (˜200 μg) of protein was diluted in 100 μL of 8 M urea, 100 mM Tris-HCl, pH 8.5 (UA buffer) and loaded onto a 10 kDa molecular weight cut-off (MWCO) centrifugal filter (Merck Millipore, Australia) and centrifuged at 20,800 g for 15 min at room temperature (RT). The filter with retained proteins >10 kDa was washed with 200 μL of UA buffer and centrifuged at 20,800 g for 15 min at RT. To chemically reduce bisulfide bonds in the protein on the filter, 200 μL of 50 mM dithiothreitol solution was added and the mixture incubated at room temperature for 50 min with shaking. The filter was washed with two 200 μL volumes of UA buffer with centrifugation each time at 20,800×g for 15 min. For cysteine alkylation, 100 μL of iodoacetamide (IAM) solution (50 mM IAM in UA buffer) was added and the mixture incubated in the dark for 30 min at RT before centrifugation (20,800g, 15 min). The retained protein was washed with two 200 μL volumes of UA buffer with centrifugation (20,800×g, 15 min) followed by two subsequent wash/centrifugation steps with 200 μL of 50 mM ammonium bicarbonate. 200 μL of the trypsin (sequencing grade, Promega, Alexandria, Australia) solution (20 μg/mL in 50 mM ammonium bicarbonate and 1 mM CaCl₂) was loaded onto the filter and incubated for 1 h or 18 h at 37° C. in a wet chamber. The tryptic peptides were collected by centrifugation (20,800×g, 15 min) followed by an additional wash with 200 μL, of 50 mM ammonium bicarbonate. The combined filtrates were lyophilised and stored at ˜20° C.

### LC-MS Analysis of Proteins

The trypsin digested peptides were dissolved in 50 μL of 1% formic acid (FA) and a 4 μL aliquot loaded onto an Ekspert nanoLC415 (Eksigent, Dublin, CA, U.S.A.) for chromatographic separation, directly coupled to a 6600 TripleTOF MS (SCIEX, Redwood City, CA, USA). The peptides were desalted for 5 min on a ChromXP C18 (3) μm, 120 Å, 10 mm×0.3 mm) trap column at a flow rate of 10)EL/min using 0.1% FA, and separated on a ChromXP C18 (3 μm, 120 Å, 150 mm×0.3 mm) column at a flow rate of 5 μL/min at 30° C. A linear gradient from 3-25% solvent B over 68 min was employed followed by: 5 min from 25% B to 35% B; 2 min 35% B to 80% B; 3 min at 80% B, 80-3% B, 1 min; and 8 min re-equilibration. The solvents were: (A) 5% DMSO, 0.1% FA, 94.9% water; (B) 5% DMSO, 0.1% FA, 90% acetonitrile, 4.9% water. The instrument parameters were: ion spray voltage 5500 V, curtain gas 25 psi, GS1 15 psi and GS2 15 psi, heated interface 150° C. Data were acquired in information-dependent acquisition (IDA) mode comprising a time-of-flight (TOF)-MS survey scan followed by 30 MS/MS, each with a 40 ms accumulation time. First stage MS analysis was performed in positive ion mode, mass range m/z 400-1250 and 0.25 s accumulation time. Tandem mass spectra were acquired on precursor ions >150 counts/s with charge state 2-5 and dynamic exclusion for 15 s with a 100 ppm mass tolerance. Spectra were acquired over the mass range of m/z 100-1500 using the manufacturer's rolling collision energy (CE) based on the size and charge of the precursor ion. For proteins extracted from E. coli, protein identification was undertaken using ProteinPilot™ 5.0 software (SCIEX) with searches conducted against the E. coli subset of the Uniprot database appended with a custom nitrogenase (Nif+Mit2Nif) database including the control chloramphenicol resistance protein (CAT/P62577) and a contaminant database (Common Repository of Adventitious Proteins). For proteins extracted from N. benthamiana, the searches were conducted using a N. benthamiana subset of the Uniprot database appended with the custom nitrogenase (Nif+Mit2Nif) database and the contaminant database (Common Repository of Adventitious Proteins).

From the identified peptides, two NifM peptides, namely DAFAPLAQR (SEQ ID NO:155) and DYLWQQSQQR (SEQ ID NO:156) that were fully tryptic, contained no unusual cleavages and/or modifications and showed high response in the MS as judged by peak intensity, were selected for multiple reaction monitoring (MRM) scanning to confirm the detection of the nitrogenase (NifM) proteins in the E. coli JM109 expression system.

The enzyme chloramphenicol acetyltransferase (CAT; P62577), which provides chloramphenicol resistance in bacteria, was expressed from the selectable marker gene in all of the transformed E. coli (strain JM109) cells containing the modified or unmodified pMIT2.1 genetic constructs. This polypeptide was therefore selected as a control to standardize protein expression levels. Three tryptic peptides (four transitions/peptide) from CAT polypeptide were selected to measure the level of CAT, namely ITGYTTVDISQWHR (SEQ ID NO:157), LMNAHPEFR (SEQ ID NO:158) and YYTQGDK (SEQ ID NO:159).

### Targeted Liquid Chromatography-Multiple Reaction Monitoring-Mass Spectrometry (LC-MRM-MS)

Reduced and alkylated tryptic peptides (5 μL) were chromatographically separated on a Kinetex C18 column (2.1 mm×100 mm, Phenomenex) using a linear gradient of 5-45% acetonitrile in 0.1% formic acid, over 10 min at a flow rate of 400 μL/min. The eluent from the Shimadzu Nexera UHPLC was directed to a QTRAP 6500 mass spectrometer (SCIEX) equipped with a TurboV ionisation source operated in positive ion mode for data acquisition and analysis. The MS parameters were as follows: ion spray voltage, 5500 V; curtain gas, 35; GS1, 35; GS2, 40; source temperature, 500° C.; declustering potential, 70 V; and entrance potential, 10 V. Peptides were fragmented in the collision cell with nitrogen gas using rolling collision energy dependent on the size and charge on the size and charge of the precursor ion. Relative quantitation using scheduled multiple reaction monitoring (MRM) scanning experiments with a 40 second detection window around the expected retention time (RT) and a 0.3 second cycle time. Data were acquired using Analyst v1.7 software. Peak areas of four MRM transitions were integrated using Skyline (MacLean, Bioinformatics 2010) wherein all transitions were required to co-elute with a signal-to-noise (S/N)>3 and intensity >1000 counts per second (cps) for detection.

Acetylene Reduction Assays Using the pMIT2.1 System in E. coli

Cells of E. coli strain JM109 were transformed with the plasmids pMIT2.1 (or one of its derivatives that was being tested) and the controller plasmid pN249 which conferred resistance to the antibiotics chloramphenicol and spectinomycin, respectively, as described in Temme et al., 2012. The transformed cells were selected by growth on LB medium (10 g/L tryptone, 5 g/L yeast extract, 10 g/L NaCl) containing chloramphenicol (34 mg/L) and spectinomycin (80 mg/L). Transformed cells were grown aerobically overnight at 37° C. in LB medium with antibiotics to an optical density at 600 nm of 1.0. The cultures were centrifuged at 10,000 g for 1 minute and the supernatant discarded. The cells were re-suspended in one volume of an induction medium which was free of N sources, containing 25 g/L Na₂HPO₄, 3 g/L KH₂PO₄, 0.25 g/L MgSO₄·7H₂O, 1 g/L NaCl, 0.1 g/L CaCl₂·2H₂O, 2.9 mg/L FeCl₃, 0.25 mg/L Na₂MoO₄·2H₂O and 20 g/L sucrose (minimal medium) supplemented with 1.5 ml/L of 10% serine, 600 μl/L 0.5% Casamino acids, 5 mg/L biotin and 10 mg/L para-aminobenzoic acid (Yang et al., 2018). The medium was sparged with argon gas for 20 minutes prior to mixture with the bacteria and antibiotics. Stock solutions were filter sterilized. For induction of Nif gene expression, the medium was supplemented with isopropyl-μ-D-1-thiogalactopyranoside (IPTG; Gold Bio #I2481C25 259) at a final concentration of 0.1 mM, 0.5 mM or 1.0 mM unless otherwise stated, generally 1.0 mM. The cell suspensions were transferred to 3.5 cc culture flasks and capped with gas-tight rubber seals using a crimp-lock system and the headspace was sparged with pure argon gas for 20 min. The suspensions were then incubated at 30° C. with shaking at 200 rpm for 5 hours. After this, acetylene reduction assays (ARA) were started by the injection of 0.5 cc of pure C2H2 (BOC gases, instrument grade; final concentration 10% C2H2 in argon) and further incubation for 18 hours. Production of ethylene at the final time was measured by gas chromatography with flame ionisation detection (GC-FID) using an

Agilent 6890N GC instrument. Headspace samples (0.5 cc) were removed and manually injected into a split/splitless inlet on a 10:1 split mode. The instrument was operated under the following parameters: inlet and FID temperatures of 200° C., average velocity for the carrier He of 35 cm/sec, isothermal oven temperature at 120° C. A RT-Alumina Bond/MAPD column (30 m×0.32 mm×5 μm) was used with a 5 m particle trap column coupled to the detector end. Analytical performance of the instrument was assessed by running suitable blanks and standards. Under these conditions, ethylene emitted from the column at about 2.3 minutes and acetylene at about 3.1 minutes. This GC system was able to detect ethylene at levels as low as 0.00001% atm with clear resolution from acetylene as the only other detectable peak in this format, so was extremely sensitive.

The assay system using wild-type pMIT2.1 and pN249 in E. coli strain JM109 as the positive control produced only a trace level of ethylene when no IPTG was added to the growth medium, whereas addition of IPTG to the growth medium at 0.1 mM, 0.5 mM or 1.0 mM greatly increased the amount of ethylene produced. The rate of ethylene production increased greatly from 3 hr sampling to 18 hr, and also as the IPTG concentration was increased, indicating increased nitrogenase activity with increased Nif gene expression. Therefore, the assays generally used 1.0 mM IPTG and sampling after 18 hr.

### Yeast Transformation and Culture Conditions for Recombinant Protein Expression

Transformation of yeast strain INVSc1 (Thermo Fisher Scientific) was performed using the Yeast Transformation Kit (Sigma Aldrich) according to the manufacturer's protocol. For vectors having a Ura gene as selectable marker, transformed colonies were selected by plating the transformation mixture onto minimal medium without uracil (SCMM-U) agar plates, which contained 6.7 g/L yeast nitrogen base, 1.92 g/L synthetic dropout medium without uracil (Sigma Aldrich), 20 g/L glucose, and 20 g/L agar. After 2-3 days of incubation at 30° C., single colonies were restreaked onto fresh SCMM-U agar plates. The presence of the genetic construct including a NifD gene or other Nif was confirmed by PCR using gene specific primers. A single colony that contained the genetic construct was inoculated into SCMM-U liquid media (containing the same components as SCMM-U agar but without the agar), grown at 30° C. with shaking for 2 days. Glycerol was added to a final concentration of 20% and aliquots stored in −80° C. until further use.

For expression of the genes contained in the genetic construct, an inoculant from the glycerol stock was grown in SCMMM-U liquid media at 30° C. with shaking for 2 days. The cells were collected from the culture by centrifugation and resuspended in SCMM-U induction medium which was identical to SCMM-U liquid media except that the glucose was replaced with 20 g/L galactose, to a final OD600 of 0.4. The culture for induction was grown at 30° C. with shaking for 2 days and the yeast cells were collected by centrifugation for protein extraction and Western blot analysis.

### Example 2. Production of Nif Polypeptides in Mitochondria of Plant Cells by Expressing MTP-Nif Fusion Polypeptides

The inventors have previously reported the successful production of 16 different Nif polypeptides in mitochondria of plant cells by introducing chimeric genes encoding translational fusions of mitochondrial targeting peptides (MTP) linked to the N-termini of the Nif polypeptides (Allen et al., 2017; WO2018/141030). The MTP sequences used included one of 77 amino acids in length (amino acids 1-77 of SEQ ID NO:20) derived from an A. thaliana F1-ATPase (At2G33040; Lee et al, 2012) and designated herein as MTP-FAγ77, which, with a 3-amino acid linker of Gly-Ala-Pro (GAP) linking the MTP to the N-terminus of the Nif polypeptides, provided an 80 amino acid N-terminal extension to the translated Nif polypeptides. Cleavage by MPP occurred after 42 amino acids, leaving an N-terminal extension of 38 amino acid residues fused to the Nif polypeptide of interest, 35 residues coming from MTP-FAγ77 plus the GAP. This N-terminal extension was termed FAγ-scar38. The natural translation initiation methionine residue of each Nif polypeptide was thereby replaced by the scar-38 sequence. Those experiments did not test for the normal function of the Nif polypeptides that were produced.

The inventors sought to shorten the MTP sequence from the 77 amino acids of MTP-FAγ77 for use with Nif polypeptides in plant cells, while still retaining MTP function. The inventors examined whether 26 amino acids could be trimmed from the C-terminus of MTP-FAγ77 to generate an MTP designated as MTP-FAγ51 (SEQ ID NO:21). This sequence had a C-terminal GG added as a consequence of the cloning procedure. The inventors predicted that MTP-FAγ51 would be cleaved by MPP after amino acid 42, leaving 9 amino acids (ISTQVVRNR; SEQ ID NO:22) from MTP-FAγ51 at the N-terminus of the processed fusion polypeptide and a linking GG as a result of the cloning procedure. This 9-amino acid sequence was designated as FAγ-scar9 or simply as scar9.

To test the function of MTP-FAγ51 relative to the longer version, a genetic construct was first made encoding this MTP fused to NifH. The modified NifH gene was identical to the NifH gene in pRA10 (encoding MTP-FAγ77+GAP::NifH::HA; SEQ ID NO:23) except that the encoded polypeptide had MTP-FAγ51 fused to the N-terminus of NifH rather than MTP-FAγ77. The polypeptide still included the GAP amino acids for cloning purposes. The NifH coding region in both constructs was codon optimised for expression in human cells, based on the nucleotide sequence in pRA10. Both constructs included a sequence encoding a HA epitope tag at the C-terminus of the Nif polypeptide to provide for detection and purification of the polypeptides with HA-antibody. The shortened construct was designated pRA34 (encoding MTP-FAγ51+GAP::NifH::HA, SEQ ID NO:24).

A second construct was made, designated SN18 and encoding a NifH fusion polypeptide having the amino acid sequence provided as SEQ ID NO:25, which included several modifications relative to pRA34 aimed at increasing expression levels. An enhanced 35S promoter (e35S; Kay et al., 1987) was used to express the fusion protein, an additional N-terminal Met was added as a translation start, a TMV 5′-UTR was added upstream of the protein coding region and the codon usage was switched to A. thaliana codon usage. All of these modifications were made to increase the expression level at both the transcriptional and translational level. Additionally, the amino acids GG were used instead of GAP immediately after the MTP. A third construct was also made, designated SN29 and encoding a NifH fusion polypeptide having the amino acid sequence provided as SEQ ID NO:26, where the polypeptide had the HA epitope tag immediately after the MTP-FAγ51 sequence (SEQ ID NO:36) and before the GG and NifH sequences (MTP-FAγ51::HA::NifH). Both of these constructs were made by GoldenGate cloning methods (Weber et al., 2011) which provided for assembly of genetic components into the construct in a modular fashion with specific components as described by Engler (2014).

These constructs were tested in the N. benthamiana leaf system and compared to the longer construct pRA10. Protein extracts were produced from the infiltrated leaf tissues and subjected to SDS PAGE and Western blot analysis using HA-antibody to assess protein expression levels and MPP processing efficiency. As a control for the size of the unprocessed fusion polypeptides, protein extracts from E. coli expressing pRA34 and pRA10 were run in adjacent lanes on the gels. The bacterial extracts yielded polypeptide bands of the expected sizes for unprocessed MTP::NifH. In contrast, protein extracts from the N. benthamiana leaf tissues infiltrated with these constructs yielded polypeptide bands of smaller sizes, corresponding to the sizes expected for the MPP-processed polypeptides. Expression of the MTP-FAγ51::NifH::HA from pRA34 and SN18, and MTP-FAγ51::HA::NifH polypeptide from SN29, each yielded a band at a smaller MW than MTP-FAγ77+GAP::NifH::HA in accordance with the difference in expected size between the polypeptides, due to the shortened MTP sequence. Expression from SN18 was at least as strong as from pRA34; both were strong. The inventors concluded that the shortened MTP-FAγ51 was capable of directing a synthetic NifH fusion polypeptide to the mitochondria of plant cells and provided for processing by MPP in the mitochondria.

Based on the success with pRA34, SN18 and SN29 encoding NifH polypeptides, the shorter MTP sequence was tested with the other 15 Nif polypeptides, encoding the corresponding MTP-FAγ51 versions. A series of genetic constructs were made (Tables 3 and 4) for this using the GoldenGate approach (Weber et al., 2011). The GoldenGate cloning system was used for assembling the different gene elements, including the promoters, 5′-UTR, 3′-UTR, N- and C-terminal extensions and terminators. Each element had defined boundaries that allowed for modular assembly and easy exchange of elements. This cloning system with components as described by Engler et al. (2014), was therefore used for testing a large variety of different genetic constructs for production of MTP::Nif fusion polypeptides in the following Examples. Since the GoldenGate cloning system utilised type IIS restriction enzymes that cut outside their recognition sequence, it was possible to avoid the use of restriction enzyme cloning sites within the junction sequences. This allowed construction of genes encoding MTP::Nif fusions without the Gly-Ala-Pro sequence present in the earlier constructs. As above, a Gly-Gly bridge at the junction of the MTP::polypeptide fusions was used instead, to fit the GoldenGate system. Glycine was chosen as the standard amino acid for this linkage due to its common occurrence at the −1 position of MTP sequences. As an exception to this, the construct to express the NifK fusion polypeptide (SN140) had the HA epitope inserted between the MTP-FAγ51 and the NifK sequence, separated by a Gly-Gly bridge, and a wild-type C-terminal end. This variation was made as it had been observed previously that the NifK polypeptide required a wild-type C-terminus, with no C-terminal extension, for activity (WO2018/141030).

A second, parallel set of genetic constructs was made which encoded cytoplasmically localized Nif polypeptides rather than mitochondrially localized polypeptides. This was done by replacing the MTP-FAγ51 encoding sequence in the constructs with a nucleotide sequence encoding a 6×His amino acid motif (SEQ ID NO:27). The 6×His motif was of a similar molecular weight to the FAγ-scar9 motif resulting from MPP-mediated cleavage of the MTP-FAγ51 sequence. The polypeptides fused to 6×His were otherwise identical to the MTP-FAγ51::Nif::HA polypeptides, including the presence of the C-terminal HA epitope. Thereby, the 6×His versions of the polypeptides provided suitable molecular weight controls to the corresponding MPP-processed FAγ-scar9::Nif::HA polypeptides on the Western blots. The exception to this was the control (cytoplasmic localised) construct to express NifK (SN72) which had an N-terminal fused HA epitope, without an MTP sequence, rather than the 6×His motif. The genetic constructs and the predicted molecular weight of the fusion polypeptides for the unprocessed MTP-FAγ51::Nif::HA polypeptides and the 6×His fusion polypeptides are listed in Table 3.

TABLE 3
Plasmids were constructed to test mitochondrial targeting efficiency
and protein solubility of Nif polypeptides expressed in N. benthamiana
leaves. The plasmids encoding cytoplasmically localized polypeptides
had a 6xHis motif instead of the MTP sequence. The predicted polypeptide
sizes (kDa) were calculated using VNti software.

| | Mitochondrially targeted | | | Cytoplasmically localized | |
|---|---|---|---|---|---|
| | Construct | Polypeptide | Molecular weight | | Molecular |
| Nif | ID | description | (kDa) | Construct ID | weight (kDa) |
| B | SN192 | pFAγ51::NifB::HA | 59 | SN201 | 54 |
| D | SN10 | pFAγ51::NifD::HA | 61.1 | SN196 | 56.3 |
| E | SN38 | pFAγ51::NifE::HA | 57.2 | SN203 | 52.4 |
| F | SN138 | pFAγ51::NifF::HA | 26.2 | SN204 | 21.4 |
| H | SN18/SN27 | pFAγ51::NifH::HA | 39.0 | SN205 | 34.2 |
| J | SN139 | pFAγ51::NifJ::HA | 135.2 | SN206 | 130.4 |
| K | SN140 | pFAγ51::HA::NifK | 65.3 | SN72 | 59.7 |
| M | SN30 | pFAγ51::NifM::HA | 37.7 | SN207 | 32.9 |
| N | SN39 | pFAγ51::NifN::HA | 57.6 | SN208 | 52.8 |
| Q | SN141 | pFAγ51::NifQ::HA | 26.7 | SN209 | 21.9 |
| S | SN31 | pFAγ51::NifS::HA | 50.3 | SN210 | 45.5 |
| U | SN32 | pFAγ51::NifU::HA | 36.5 | SN211 | 31.7 |
| V | SN142 | pFAγ51::NifV::HA | 48.2 | SN212 | 43.4 |
| W | SN143 | pFAγ51::NifW::HA | 17.2 | SN213 | 12.4 |
| X | SN144 | pFAγ51::NifX::HA | 25.3 | SN214 | 20.5 |
| Y | SN145 | pFAγ51::NifY::HA | 31.5 | SN215 | 26.7 |
| Z | SN146 | pFAγ51::NifZ::HA | 23.7 | SN216 | 20.9 |

The NifD and NifS polypeptide sequences used in these fusions were the sequences according to Temme et al. (2012). These amino acid sequences are provided in SEQ ID NO:18 and SEQ ID NO:19, respectively. The NifD amino acid sequence of SEQ ID NO:18 differed from the sequence of 483 amino acids provided as SEQ ID NO:2 by six amino acid substitutions, at positions 39, 41, 87, 96, 355 and 483. The NifS amino acid sequence of SEQ ID NO:19 differed from the sequence of 400 amino acids provided as SEQ ID NO:11 by four amino acid substitutions, at positions 110, 113, 124 and 290. All of the genetic constructs designated herein with an SN number which contained a NifD sequence or a NifS sequence used the sequences according to Temme et al. (2012).

Each of the constructs was introduced into N. benthamiana leaf cells and, after 5 days, proteins extracted from the infiltrated leaf tissue and analysed by the Western blot method. Samples from the constructs expressing the 6×His polypeptides, included as molecular weight markers on the Western blots for the corresponding MPP-processed FAγ-scar9::Nif::HA polypeptides (see Table 3), were electrophoresed in adjacent lanes on the gels. Detection of the polypeptides used the HA epitope fused to the C-terminus of each Nif polypeptide.

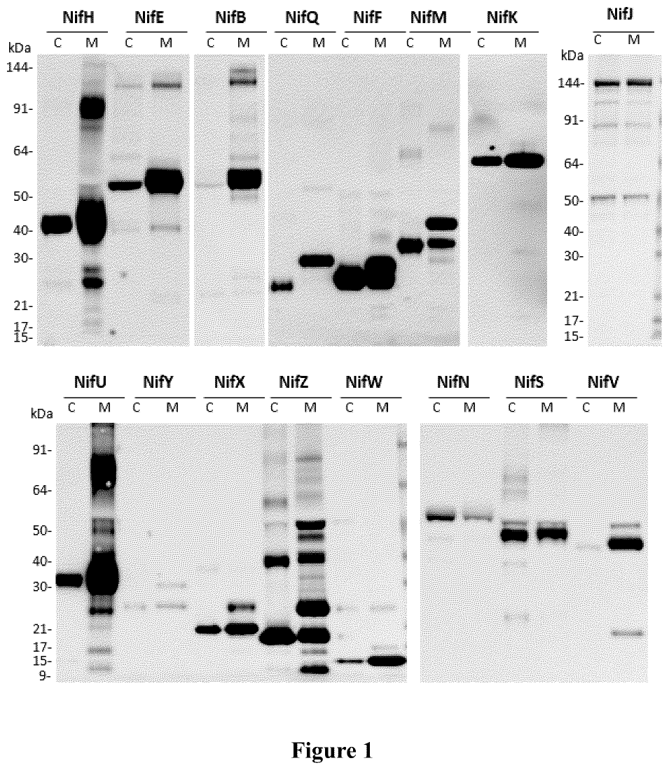

The results for processing by MPP are shown in FIG. 1 and summarised in Table 4. When translationally fused as a MTP::Nif fusion, MTP-FAγ51 yielded a cleaved MTP::Nif polypeptide for almost all of the Nif polypeptides, but not all with equal efficiency. NifQ was the least processed, with only a trace of the processed form detected when total protein was analysed by Western blot in one experiment, none in another. The NifF, NifM, NifV, NifX, NifY and NifZ fusion polypeptides were only partially processed when fused to FAγ51, whereas the other Nif fusion polypeptides including NifB, NifE, NifK, NifN, NifS, NifU and NifW were processed efficiently, demonstrating that processing efficiency for different Nifs can vary for the one MTP. The NifD fusion polypeptide was detected at low levels but consistently showed a degradation product (see below). As for relative levels of expression, the genetic construct encoding NifY yielded lower levels of the polypeptide than the others with the exception of NifD. It was thought this was due to lower expression levels of the NifY gene such as lower translation rates compared to the other Nif proteins and/or instability of the polypeptides. Fusing the NifY coding region with a different MTP than FAγ51 is one approach to improving levels of polypeptide accumulation.

A difference was observed for some Nif polypeptides in the amount of the cytoplasmically localized polypeptide (6×His) relative to the mitochondrially-localized polypeptide. In particular, the NifB, NifE, NifH, NifU and NifV polypeptides targeted to the mitochondria accumulated to a greater level than the corresponding polypeptide targeted to the cytoplasm, whereas the level of accumulation of the other Nif polypeptides was approximately equal between the mitochondrial and cytoplasmic forms. The only exception to this trend was NifN, where the cytoplasmically-targeted polypeptide accumulated to a greater level than the mitochondrial counterpart.

Some higher-molecular weight polypeptide bands were also observed in the Western blots from constructs encoding NifE, NifH, NifB, NifU and NifZ (FIG. 1). It was considered that those bands might correspond to dimerization complexes that were resistant to the strong denaturing conditions used in the sample preparation. Previously, similar high-molecular weight bands were observed for various mitochondrially-targeted Nif proteins using a different MTP (Allen et al., 2017).

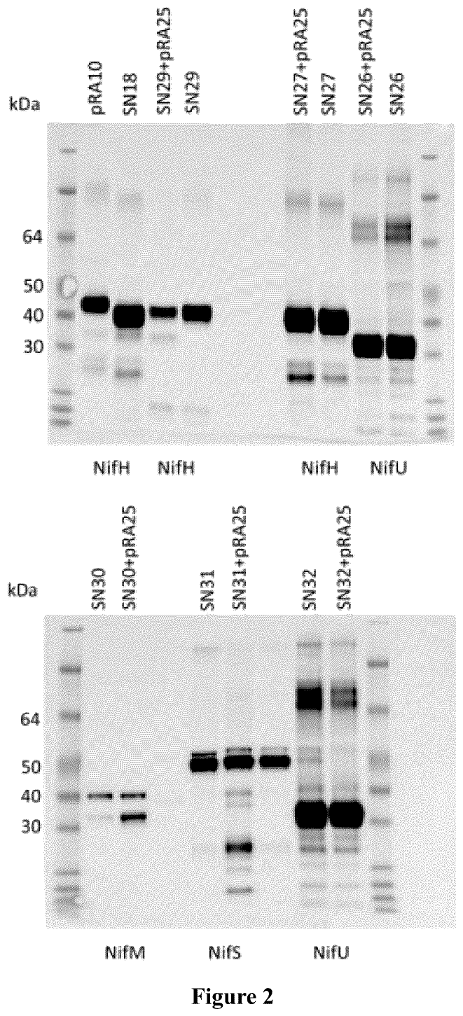

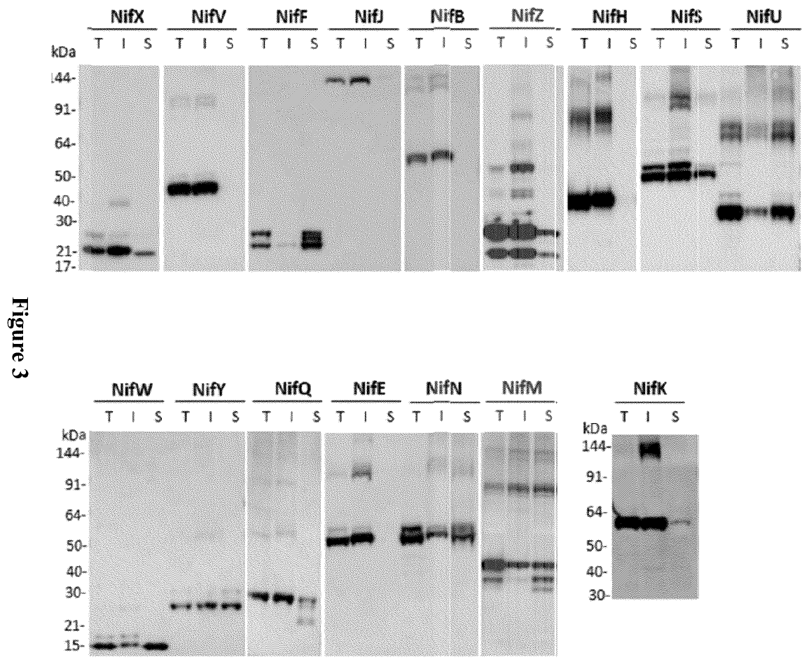

A Western blot comparing pRA10 and SN18 is shown in FIG. 2, as well as constructs encoding NifH, NifM, NifS and NifU. The samples for FIG. 2 included proteins extracted from paired infiltrations either with or without co-infiltration of pRA25 which encodes a MTP-FAγ77::NifK fusion polypeptide (WO2018/141030), in order to test whether addition of NifK would affect expression and/or MPP-processing. No differences were observed by adding NifK for expression and processing of NifH, NifM, NifS and NifU.

It was concluded from these experiments that the MTP-FAγ51 amino acid sequence was capable of targeting all of the Nif polypeptides to the mitochondrial matrix in plant cells and provided for processing by MPP, with the exception of processing of the NifQ polypeptide. The polypeptide expression levels and processing efficiencies were as good as for the longer FAγ MTP. Additionally, in some cases fewer polypeptide bands of smaller sizes, thought to indicate degradation products, were detected with the HA antibody in the blots, for example for pRA34. The inventors concluded that the shorter MTP sequence could unexpectedly reduce MTP::Nif degradation.

### Alternative MTPs

A range of different MTP sequences were tested to assess their performance in translocating Nif polypeptides to the mitochondrial matrix of plant cells. Several MTPs of differing lengths (30-70 amino acid residues) were selected. These were predicted to leave different lengths of remaining amino acid residues (“scar sequence” or simply “scar”) fused at the N-termini of the Nif polypeptides after cleavage by MPP (Table 5). The scar sequences ranged from 0-36 amino acid residues in length. Using the GoldenGate cloning system, 17 different genetic constructs were assembled using combinations of these MTPs with several Nifs for expression in plant cells, particularly for expressing NifD fusion polypeptides since NifD was the most difficult of the Nif polypeptides to express (WO2018/141030). The promoters, 5′ and 3′UTRs and terminators were identical for these constructs.

A. tumefaciens cultures containing these constructs, each mixed with the construct producing P19 silencing suppressor protein, were individually introduced into N. benthamiana leaves as described in Example 1 and protein extracts produced 5 days post-infiltration. SDS-PAGE and Western blot analyses were carried out on the protein extracts. For infiltrations with the MTP::NifD construct, SN46 (pSu9::NifK) was co-infiltrated, since co-expression of NifK without a C-terminal extension had been shown to enhance NifD abundance (WO2018/141030).

Two versions were tested of the CPN60 MTP fused to NifD. In one version, the MTP was fused such that a Gly-Gly linker was placed between the CPN60 MTP (SEQ ID NO:28) and NifD (SN11). In each case where present, the Gly-Gly linker was inserted by the GoldenGate cloning procedure but could be considered to be part of the MTP sequence. In the other version (SN4), the CPN60 MTP (SEQ ID NO:29) was fused directly to the first methionine of NifD polypeptide. As CPN60 was predicted to be cleaved immediately after the C-terminal tyrosine in its amino acid sequence, this construct would theoretically produce NifD polypeptide with a wild-type N-terminus, i.e. no “scar”, whereas the SN11 construct was predicted to leave a Gly-Gly extension after cleavage of the MTP(GlyGly)::NifD fusion. Surprisingly, these very similar constructs produced differing outcomes as evidenced by Western blot analysis: SN11 yielded a polypeptide band at the size expected for unprocessed CPN60(GlyGly)::NifD, whereas SN4 yielded bands corresponding to both processed and unprocessed polypeptides, with more unprocessed polypeptide present than processed polypeptide. Furthermore, when protein from the infiltrations with SN4 was compared by Western blot to protein extracted from a parallel pRA24+pSN46 (FAγ77+GAP::NifD::HA+Su9::NifK) infiltration, it was apparent that the SN4 construct produced considerably less correctly processed polypeptide than the pRA24 construct. Therefore, it appeared that, although the CPN60 MTP was able to target the fusion polypeptide and allowed for matrix processing to produce a wild-type NifD polypeptide, the expression level and processing efficiency was low (US2016/0304842). For SN11, the Gly-Gly linkage between CPN60 and NifD may have prevented processing of the MTP.

Several MTPs derived from superoxide dismutase (SOD) polypeptides were also tested, either as single or tandem MTPs, and either with or without the inclusion of Ile and Gln at the C terminus prior to the Gly-Gly linkage. Polypeptides were not detected by Western blot analysis for the versions containing the SOD MTP (SN15, SEQ ID NO:32 and SN16, SEQ ID NO:33) that did not contain the Ile and Gln residues, whereas the versions having SOD MTP that retained the Ile and Gln residues (SN12, SEQ ID NO:30 and SN13, SEQ ID NO:31) did produce detectable polypeptides, although it appeared that they were not processed by the MPP. In contrast, another MTP tested, L29 (SN17, SEQ ID NO:34), yielded strong polypeptide signals when fused to NifD. Due to the small difference in size between processed and unprocessed forms with this MTP, additional experiments will be required to determine processing efficiency. It is expected that the L29 MTP yielded cleaved Nif polypeptide in an efficient manner. The inventors also tested a CoxIV MTP with a twin strep tag (Buren et al., 2017) fused at the C terminus of the MTP, but upstream of the Gly-Gly linkage (SN19, SEQ ID NO:37). This MTP when fused to NifD also gave strong signals by Western blot analysis, of a size consistent with mitochondrial matrix processing.

TABLE 4
Summary of level of MPP processing of MTP::Nif polypeptides,
solubility within plant mitochondria and functionality of scar9-
Nif in a bacterial assay. Yes (p) indicates partial (less than 50%)
MPP-processing. Solubility scores for processed Nifs are: −,
Insoluble or slightly soluble; +, partially soluble, ++
mostly or fully soluble. Nt: not tested. Functional
testing was in E. coli JM109 using MIT2.1 vector system.

| Con- | | | SEQ ID NO | MTP | Solu- | % Func- |
|---|---|---|---|---|---|---|
| struct | | | of fusion | Pro- | bility | tion in |
| ID | Nif | MTP | polypeptide | cessed | in MM | JM109 |
| SN192 | B | FAγ51 | 147 | Yes | − | 100 |
| SN10 | D | FAγ51 | 122 | Yes (p) | − | 50 |
| SN38 | E | FAγ51 | 126 | Yes | − | 30 |
| SN138 | F | FAγ51 | 137 | Yes (p) | ++ | 100 |
| SN18/ | H | FAγ51 | 25 | Yes | − | 100 |
| SN27 | | | | | | |
| SN42 | H | CoxIV-twin | 128 | Yes | − | Nt |
| | | strep | | | | |
| SN139 | J | FAγ51 | 138 | Yes | − | 200 |
| SN140 | K | FAγ51 | 139 | Yes | − | 70 |
| SN30 | M | FAγ51 | 123 | Yes (p) | ++ | 10 |
| SN39 | N | FAγ51 | 127 | Yes | + | 50 |
| SN141 | Q | FAγ51 | 140 | trace | + | 100 |
| SN31 | S | FAγ51 | 124 | Yes | + | 50 |
| SN32 | U | FAγ51 | 125 | Yes | ++ | 80 |
| SN142 | V | FAγ51 | 141 | Yes | − | 90 |
| SN51 | V | L29 | 130 | Yes (p) | − | Nt |
| SN104 | W | Su9 | 135 | Yes | ++ | Nt |
| SN143 | W | FAγ51 | 142 | Yes | + | 85 |
| SN144 | X | FAγ51 | 143 | Yes (p) | + | Nt |
| SN145 | Y | FAγ51 | 144 | Yes (p) | + | 60 |
| SN146 | Z | FAγ51 | 145 | Yes (p) | + | 60 |

TABLE 5
Details of MTPs used for testing in plants using the GoldenGate system.
kDa F/P: full length size of unprocessed MTP/processed size of MTP in
kDa. Scar = predicted amino acid sequence of the remaining N-terminal
extension after MPP processing.

| | | kDa | |
|---|---|---|---|
| MTP Name | MTP | F/P | Scar |
| gATPase gamma | MAMAVFRREGRRLLPSIAARPI | 5.75/ | ISTQVVRNRGG (SEQ ID |
| subunit (FAγ51); | AAIRSPLSSDQEEGLLGVRSIST | 1.19 | NO: 42) |
| (Huang et al., 2009) | QVVRNRGG (SEQ ID NO: 36) | | |
| CPN60 | MYRFASNLASKARIAQNARQV | 3.77/ | GG |
| (At2G33040) | SSRMSWSRNYGG (SEQ ID | 0.13 | |
| (Prasad and Stewart, | NO: 28) | | |
| 1992) | | | |
| CPN60/No GG linker | MYRFASNLASKARIAQNARQV | 3.65/ | none |
| (At2G33040); | SSRMSWSRNY (SEQ ID NO: 29) | 0 | |
| (Prasad and Stewart, | | | |
| 1992) | | | |
| Superoxide | MAIRCVASRKTLAGLKETSSRL | 3.34/ | IQGG (SEQ ID NO: 39) |
| dismutase (SOD) | LRIRGIQGG (SEQ ID NO: 30) | 0.37 | |
| (At3G10920) | | | |
| (Huang et al., 2009) | | | |
| Superoxide | MAIRCVASRKTLAGLKETSSRL | 6.65/ | IQGG (SEQ ID NO: 39) |
| dismutase doubled | LRIRGIQMAIRCVASRKTLAGL | 0.37 | |
| (2SOD) | KETSSRLLRIRGIQGG (SEQ ID | | |
| (At3G10920); | NO: 31) | | |
| (Huang et al., 2009) | | | |
| Superoxide | MAIRCVASRKTLAGLKETSSRL | 3.1/ | GG |
| dismutase modified | LRIRGGG (SEQ ID NO: 32) | 0.13 | |
| (SODmod) | | | |
| (At3g10920); | | | |
| (Marques et al., | | | |
| 2014) | | | |
| Superoxide | MAIRCVASRKTLAGLKETSSRL | 6.07/ | GG |
| dismutase modified | LRIRGMAIRCVASRKTLAGLKE | 0.13 | |
| (2SODmod) doubled | TSSRLLRIRGGG (SEQ ID NO: 33) | | |
| (At3g10920) | | | |
| (Marques et al., | | | |
| 2014) | | | |
| L29 (At1G07830) | MFLTRFVGRRFLAAASARSEST | 3.5/ | ESTTAAAAASTIRGG |
| (Huang et al., 2009) | TAAAAASTIRGG (SEQ ID | 1.36 | (SEQ ID NO: 40) |
| | NO: 34) | | |
| Neurospora crassa | MASTRVLASRLASQMAASAKV | 7.5/ | YSGG (SEQ ID NO: 41) |
| F0 ATPase subunit 9 | ARPAVRVAQVSKRTIQTGSPLQ | 0.38 | |
| MTP (SU9); (Buren | TLKRTQMTSIVNATTRQAFQK | | |
| et al., 2017) | RAYSGG (SEQ ID NO: 35) | | |
| CoxIV twin strep | MLSLRQSIRFFKPATRTLCSSRY | 6.61/ | QQKPSAWSHPQFEKGG |
| (ABM97483) | LLQQKPSAWSHPQFEKGGGSG | 3.64 | GSGGGSGGSAWSHPQFE |
| (Burén et al., 2017) | GGSGGSAWSHPQFEKGG (SEQ | | KGG |
| | ID NO: 37) | | (SEQ ID NO: 43) |
| CoxIV 10xHis | MLSLRQSIRFFKPATRTLCSSRY | 5.07/ | QQKPGGHHHHHHHHHH |
| (ABM97483) | LLQQKPGGHHHHHHHHHHGG | 1.84 | GG (SEQ ID NO: 44) |
| (unpublished) | (SEQ ID NO: 38) | | |

### Example 3. Solubility of Nif Fusion Polypeptides in Plant Mitochondria

Solubility of nitrogenase protein components in the mitochondrial matrix is considered to be a prerequisite for functional reconstitution of nitrogenase in the mitochondria of plant cells. Although Nif polypeptides such as NifD are soluble in nitrogen fixing bacteria, it was not known if expression of synthetic MTP::NifD fusion polypeptides in plant cells would provide soluble polypeptides that could associate with the other Nif components, particularly in the mitochondrial matrix. Insolubility could be a consequence of many factors, including formation of aggregates and association with cell membranes, and would likely prevent function.

The inventors therefore evaluated the MTP-FAγ51::Nif::HA polypeptides and several others for solubility after expression of the genetic constructs (see Table 4) in N. benthamiana leaf cells. Protein extracts for the soluble and insoluble fractions were prepared as described in Example 1 as well as the unfractionated “total protein” samples which included both the soluble and insoluble proteins. The buffer for the preparation of the soluble fraction contained the non-ionic detergent Tween®20 which was added to lyse membranes and release mitochondrial matrix proteins. That mild non-ionic detergent was considered unlikely to denature Nif polypeptides. In contrast, the proteins of the insoluble fraction were solubilised prior to gel electrophoresis with a buffer containing a relatively high concentration of SDS, a strong anionic detergent that is known to denature proteins efficiently, and treatment with high temperature. The samples were then subjected to gel electrophoresis and Western blotting using anti-HA antibody to detect the polypeptides on the blots.

Several observations were made to test whether the method properly distinguished soluble and insoluble proteins. Coomassie staining of the remaining polypeptides on the gels post-transfer showed that Rubisco was present in the soluble fraction, as expected. Only trace amounts of Rubisco were found in the insoluble fraction. Western blots were also analysed with an isocitrate dehydrogenase (IDH) antibody. IDH is an oxidoreductase that participates in the citric acid cycle and is known to be located in the mitochondrial matrix and soluble. The Western blots showed the presence of IDH in the soluble fraction, indicating that mitochondria were successfully lysed and mitochondrial matrix proteins expected to be soluble were indeed present in the soluble fraction. These observations indicated that soluble proteins were successfully extracted and fractionated into the soluble sample by the methods used.

The method was then applied to the Nif fusion polypeptides; a representative Western blot is shown in FIG. 3 and the results are summarised in Table 4. The abundance of MPP-processed Nif polypeptides in the soluble fraction varied for the different pFAγ51::Nif::HA. The following MPP-processed polypeptides from the MTP-FAγ51::Nif::HA fusion polypeptide translation products appeared to be soluble or mostly soluble in the mitochondria: NifF, NifM and NifU. For the other fusion polypeptides, NifN, NifQ, NifS, NifW, NifY and NifZ were partly soluble/partly insoluble. The following appeared to be insoluble or only slightly soluble: NifB, NifD, NifE, NifH, NifJ, NifK (with a HA epitope tag N-terminal of the NifK sequence), NifV and NifX Notably, pFAγ51::NifQ::HA produced a faint band approximately the size of the correctly processed form in the soluble fraction, which was not detectable in the total protein lane. Of particular importance, each of the MTP-FAγ51::NifD::HA (from SN10), MTP-FAγ51::NifE::HA (from SN38) and MTP-FAγ51::HA::NifK (from SN140) polypeptides, when expressed on their own as a single polypeptide, were essentially insoluble—almost no mitochondrial-soluble forms of these polypeptides were detected even though considerable amounts of the polypeptides accumulated in the N. benthamiana leaf cells. For the NifH fusion polypeptides, MTP-FAγ77::NifH (from SN150) when expressed on its own as a single polypeptide was essentially insoluble, whereas only a small amount of MTP-CoxIV::twin strep::NifH (from SN42) when expressed on its own as a single polypeptide was soluble. Moreover, the MTP-FAγ51::NifD (from SN10) polypeptide, when co-expressed with the MTP-Su9::NifK polypeptide from SN46, was likewise essentially insoluble. It was concluded that each of these four, essential polypeptides for nitrogenase function were problematic in terms of solubility when expressed for import into the mitochondrial matrix.

To assess if atmospheric oxygen affected Nif protein solubility, the same 16 pFAγ51::Nif::HA proteins were isolated from infiltrated plants under anaerobic conditions as described in Example 1 and subjected to Western blot analysis as before. It was observed that anaerobic conditions during protein extraction did not significantly change the solubility of the Nif fusion polypeptides. It was concluded that the observed insolubility of some of the Nif polypeptides was not due to exposure to oxygen, even though many of the Nif polypeptides are oxygen sensitive.

Further Western blot analyses showed the following: The MTP-FAγ51::NifB::HA polypeptide (produced from SN192) was insoluble, with no band detected in the soluble fraction. NifB is also essential to nitrogenase function. The MTP-FAγ51::NifF′::HA polypeptide (SN138) was almost entirely soluble for both the polypeptides before and after MPP-processing—two bands showed on the blots which were presumed to represent MPP-processed and unprocessed forms. The MTP-FAγ51::NifJ:HA polypeptide (SN139) was essentially insoluble, with only a very faint band detected in the soluble fraction. The MTP-FAγ51::NifM::HA polypeptide (SN30) was mostly soluble after MPP-processing. For MTP-FAγ51::NifS::HA (SN31), two bands were observed on the blots which were presumed to represent MPP-processed and unprocessed polypeptides. Both were partially soluble. The MTP-FAγ51::NifV::HA polypeptide (SN142) was essentially insoluble, with only a very faint band detected in the soluble fraction. The MTP-FAγ51::NifX::HA (SN144) polypeptide was partially soluble after MPP-processing. The MTP-FAγ51::NifY::HA polypeptide (SN145) was mostly soluble, although expressed only at a low level in this experiment. The MTP-FAγ51::NifZ::HA polypeptide (SN146) was partly in the soluble fraction, partly insoluble. In this experiment, both Rubisco and IDH were present in the “total protein” and the soluble fractions and essentially absent from the insoluble fractions, showing that the method used for fractionation was effective and that soluble proteins were indeed extracted.

In an attempt to determine the cause of these solubility problems, genetic constructs encoding versions of the NifD, NifH and NifK fusion polypeptides were made which lacked an N-terminal MTP sequence. These polypeptides were predicted to be located in the cytoplasm of the plant cells, not in the mitochondria. Constructs encoding NifD (SN33), NifH (SN71), NifK (SN72) were made using GoldenGate assembly methods, each polypeptide having only a Gly-Gly linked HA epitope tag fused to the N terminus of the Nif sequence. For example, SN33 encoded a HA:NifD fusion polypeptide without the C-terminal HA epitope tag, so essentially the N-terminal MTP-FAγ51 sequence was replaced with an HA epitope sequence. Each of these three constructs was introduced separately into N. benthamiana cells via A. tumefaciens and Western blot analysis of the polypeptides carried out on soluble and insoluble protein fractions. The Western blots showed that each of the polypeptides was essentially fully soluble in the plant cells. It was concluded that the solubility problems for the NifD, NifH and NifK fusion polypeptides when fused to the MTP sequence were somehow associated with the targeting of the Nif polypeptides to plant mitochondria.

### Example 4. Functional Testing of Nif Fusion Polypeptides after MTP Cleavage

Example 2 described the production of Nif fusion polypeptides in the N. benthamiana leaf cells and delivery and processing of the fusion polypeptides in mitochondria. The fusion polypeptides were designed to have in-frame fusions of a MTP added to the N-terminus of the Nif polypeptides and an epitope tag added, sometimes as an N-terminal extension but most often as a C-terminal extension. Although modelling of protein folding and association predicted that most of the N-terminal and C-terminal extensions should not prevent complex formation and nitrogenase function, the inventors wanted to test whether these extensions might affect the function of the fusion polypeptides relative to the native Nif polypeptides. A bacterial system for testing nitrogenase function using derivatives of the pMITv2.1 vector (Smanski et al., 2014; referred to herein as pMIT2.1 or MIT2.1) was established for this. All of the wild-type genes required for nitrogenase activity were contained within the single, bacterial expression vector, pMIT2.1, where expression of the genes was controlled with an inducible promoter/T7-RNA polymerase system from a second plasmid, pN249. When expressed in E. coli, the full set of wild-type bacterial Nif polypeptides were produced and together provided a nitrogenase enzyme complex whose activity could be assayed by the production of ethylene from acetylene (acetylene reduction assay, ARA), a de facto measurement for nitrogenase activity.

This system allowed each modified polypeptide to be assayed individually, in E. coli, by addition to the otherwise wild-type nitrogenase system. This was done by replacing a Nif gene in pMIT2.1 encoding a wild-type Nif polypeptide with the corresponding, modified Nif gene encoding the Nif fusion polypeptide to be tested. Combinations of modifications to two or more Nif polypeptides could also be tested in this system. However, the pMIT2.1 vector was very large at 22,946 bp, making it unwieldy for incorporating genetic modifications. To make the pMIT2.1 vector system more workable, the MIT2.1 plasmid was first split into two halves by PCR. The first half containing the NifHDKYENJ genes was amplified using primers incorporating SbfI restriction enzyme sites at each end, namely MIT_V2.1_SbfInifH_FW2 5′-AACCTGCAGGTGACGTCTAAGAAAAGGAATATTCAGCAAT-3′ (SEQ ID NO:45) and MIT_V2.1 SbfInifJ RV2 5′-AACCTGCAGGGCTAACTAACTAACCACGGACAAA AAACC-3′ (SEQ ID NO:46), and ligated into recipient vector pCR Blunt II TOPO (Thermo Fisher Scientific), forming a vector herein designated as pTopoH-J. The second half of the Nif gene cluster containing the NifBQFUSVWZM genes was amplified using primers which also incorporated SbfI restriction enzyme sites at each end, namely MIT_V2.1_SbfInifB_FW 5′-AACCTGCAGGTACTCTAACCCCATCGGCCGTCTTA-3′ (SEQ ID NO:47), and MIT_V2.1 SbfIori RV 5′-AACCTGCAGGTACGTAGCAATCAACTCACTGGCTC-3′ (SEQ ID NO:48). This PCR product was digested with SbfI and self-ligated to form a self-replicating vector, herein designated pB-ori. To reform pMIT2.1 and its derivatives, both pTopoH-J and pB-ori, or a derivative with a modification, were digested with SbfI and the two halves of the Nif gene cluster ligated together.

As described in Example 2, the MTP-FAγ51 amino acid sequence was cleaved in plant mitochondria to leave 9 amino acid residues (FAγ-scar9; SEQ ID NO:22), plus an intervening Gly-Gly linker in the case of the SN constructs, fused to the N-terminus of the Nif polypeptide of the processed Nif fusion polypeptides. In order to test each of the fusion polypeptides for their function in the otherwise wild-type nitrogenase complex, a DNA fragment encoding the 9 amino acids except for substitution of the N-terminal Ile residue with a Met for translation initiation (MSTQVVRNR, SEQ ID NO:49, designated mscar9) was inserted immediately upstream of the translation start codon of each Nif gene in pMIT2.1 using the strategy described above. The exception was NifX since pMIT2.1 does not include NifX and therefore a modified NifX could not be tested in this system. For each construct, the DNA fragment was designed so that, when fused in-frame directly upstream of the start codon of a gene encoding any one of the Nif polypeptides, the chimeric gene would encode a translational fusion to the selected Nif polypeptide. It was expected that the translation initiation Met would be removed post-translationally in E. coli because the serine in the second position is known to promote starting Met removal by the enzyme MAP (Hirel 1989, Xiao 2010). If that happened, the resultant N-terminal extension would be of 8 amino acid residues. The removal of the starting Met residue was confirmed by enhanced production ion scanning of the target multiple reaction monitoring ion of the semi-tryptic peptide STQVVR (SEQ ID NO:50) with Q-TRAP liquid chromatography tandem mass spectrometry (see below).

For wild-type bacterial Nif polypeptides where the translation initiating Met residue was removed post-translationally in bacteria, the length of the N-terminal extension of each Nif protein was 9 amino acids with a sequence of STQVVRNRM (SEQ ID NO:51) fused to the remainder of Nif, where the terminal Met was the translation initiation amino acid of the Nif polypeptide.

As an example of the modification of pMIT2.1 and its testing, in this case to introduce a translational fusion of the nine amino acid mscar9 peptide MSTQVVRNR (SEQ ID NO:49) to the N-terminus of the Nif polypeptides, a nucleotide sequence coding for those amino acids was added to the 5′ end of a forward primer that hybridised to the 5′ end of the coding sequence for each Nif gene. For each Nif gene being modified, a reverse primer was designed adjacent to the 5′ end of the particular Nif gene. The amplified PCR product was ligated using ligation cycling reaction (LCR; de Kok et al., 2014), after which the other half of pMIT2.1 that was not modified was religated with the modified half after digestion with SbfI. For example, to introduce a translational fusion of MSTQVVRNR (SEQ ID NO:49) to the N-terminus of NifB, primers 5′-ATGTCAACTCAAGTGGTGCGTAACCGCATGACCTCTTGTTCGTCGTT-3′ (SEQ ID NO:52) and 5′-TTTAGCCCTCCTATGATTGATTTGATGTATTACAGAGAGG-3′ (SEQ ID NO:53) were used in PCR with pB-ori as template to give a 11,565 bp product. The PCR fragment was ligated by LCR with the bridging oligo 5′-GGTTACGCACCACTTGAGTTGACATTTTAGCCCTCCTATGATTGATTTGATG-3′ (SEQ ID NO:54) using the method of de Kok et al. (2014) and used to transform E. coli DH5a. The resulting construct pB-ori_scar9B was digested with SbfI and ligated to the SbfI fragment from pTopoH-J containing the unmodified NifHDKYENJ genes, yielding the modified pMIT2.1 vector encoding a fusion polypeptide having a N-terminal extension added to NifB, herein designated as pS0006. The nucleotide sequences of the resultant modified genetic constructs were confirmed to be correct by sequencing of the modified half, whether the pTopoH-J half or the pB-ori half.

Each genetic construct was introduced into E. coli strain JM109 containing pN249 and cultures of cells transformed with both vectors were grown as described in Example 1. As a negative control, pB-ori lacking 7 of the 16 Nif genes was used. An altered pMIT2.1 lacking NifM, designated ANifM was included in the experiments (cf. Lei et al., 1999; Howard et al., 1986). The transformed cells were tested for ethylene production in acetylene reduction assays after induction of gene expression with IPTG. The results summarised in Tables 4 and 5 show the percentage function in JM109 calculated as the acetylene reduction activity in E. coli JM109 containing the modified pMIT2.1 relative to that seen with JM109 containing the unmodified pMIT2.1. The control, unmodified pMIT2.1, yielded positive ethylene production. These assays showed that the addition of the 9 amino acid extension mscar9 to the N-terminus of NifB slightly increased nitrogenase function when compared to the level of ethylene production seen with the unmodified pMIT2.1.

In analogous manner, the remaining 15 Nifs also tolerated the 9 amino acid extension at their respective N-termini, with full activity for NifH, NifJ, NifQ, and NifF but with some reduction of activity for other Nifs. In a first experiment, the 9 amino acid extensions to the N-termini of NifH, NifD, NifK, NifE and NifN yielded levels of acetylene reduction activity which were 100%, 50%, 70%, 30%, and 50% compared to that of the unmodified pMIT2.1, respectively. The other Nif polypeptides, namely NifJ, NifY, NifQ, NifF, NifU, NifS, NifV, NifW, NifZ and NifM, showed 200%, 60%, 100%, 100%, 80%, 50%, 90%, 30%, 60% and 10% activity, respectively, compared to that of the unmodified pMIT2.1 (Table 4).

The experiment was repeated multiple times and the average date (n=2 to 6) is shown in Table 6. This functional testing of the individual scar9::Nif polypeptides in E. coli showed that activity was retained for all 16 Nif fusion polypeptides although there was considerable variation in activity levels for the different Nifs. Notably, scar9::NifJ had three times the activity of the positive control, and scar9::NifQ, scar9::NifH, scar9::NifB and scar9::NifF were significantly increased in ARA activity relative to the corresponding wild-type Nif polypeptides, but showing about 130-150% activity relative to the unmodified pMIT2.1 and so less than the increase observed with scar9::NifJ. In contrast, scar9::NifM only retained about 10% activity relative to the wild-type NifM. Given the high activity of scar9-NifJ (pSO028) in the pMIT2.1 system, being 2 to 3-fold more active compared to the unmodified control, the impact of modifying NifJ was further investigated. The entire NifJ region of pMIT2.1 was removed, yielding ΔNifJ-MIT2.1 (pS0014). Acetylene reduction assays with pS0014 found that its activity was similar to pMIT2.1, indicating that NifJ was redundant in the ARA assay system in JM109. Therefore, the increased activity with scar9-NifJ (pSO028) in the pMIT2.1 system may have been due to a gene dosage effect.

From the experiments described in Examples 2-4, the inventors concluded that the abundance, MPP processing and solubility of the 16 different MTP::Nif polypeptides varied, despite the use of the same MTP and promoter for each expression construct. However, all of the Nif fusion polypeptides functioned to some extent for nitrogenase activity in E. coli when the other Nif proteins were expressed as the wild-type polypeptides, indeed some with increased activity. The observed variation indicated that each Nif polypeptide had intrinsic features that influenced the amount of polypeptide that accumulated, its transport and processing by MPP. The critical components NifH and NifK were readily expressed and detected; these proteins are known to be needed at high levels for nitrogenase activity. However, they were insoluble in the leaf experiments, along with NifB, NifD, NifE, NifJ and NifV. With the exception of NifD, the NifY fusion polypeptide was expressed at the lowest level of the Nif polypeptides in these experiments. Some of the Nif polypeptides were successfully cleaved by MPP within the matrix and accumulated to higher levels relative to their cytoplasmic counterparts, suggesting that mitochondrial localization was a way of stabilizing the fusion polypeptides after cleavage by MPP. The MTP::NifQ fusion polypeptide was poorly cleaved, perhaps because the NifQ preprotein was less able to enter the mitochondrial matrix due to resistance to unfolding or mistargeting.

In these experiments, the fusion polypeptide having NifH from K. oxytoca was insoluble in the plant mitochondrial matrix. As NifM may be required for stability and solubility of NifH in bacteria (Lei et al., 1999; Howard et al., 1986) a later experiment tested a combination of mitochondrially targeted NifH and NifM in transient leaf assays.

The fusion polypeptide having K. oxytoca NifB was insoluble when mitochondrially localized, consistent with the results described for A. vinelandii NifB when targeted to yeast and plant mitochondria (Buren et al 2017a).

Considering these data together, the inventors concluded that 7 of the Nif fusion polypeptides were expressed at good levels, were processed efficiently and were localized to the mitochondrial matrix in predominantly soluble form, namely NifF, NifN, NifS, NifU, NifW, NifY and NifZ, although the abundance of NifY was relatively low. These N-terminal fusion polypeptides, after cleavage by MPP, retained reasonable levels of activity (Table 6).

TABLE 6
Effect of pFAγ51 nine amino acid ‘scar’ (scar9) peptide
translationally fused to individual Nif proteins on nitrogenase
function in E. coli. Values are presented as % acetylene
reduction activity compared to pMIT2.1. pB-ori, negative control;
ΔNifM, NifM coding sequence removed from pMIT2.1.

| | Nitrogenase activity | Standard |
|---|---|---|
| Construct ID | (% of pMIT2.1) | Deviation |
| pMIT2.1 (wild-type) | 100 | 18 |
| scar9::NifJ | 309 | 86 |
| scar9::NifQ | 158 | 22 |
| scar9::NifH | 148 | 15 |
| scar9::NifB | 144 | 34 |
| scar9::NifF | 131 | 8 |
| scar9::NifD | 110 | 9 |
| scar9::NifW | 95 | 8 |
| scar9::NifV | 81 | 11 |
| scar9::NifU | 80 | 4 |
| scar9::NifY | 65 | 4 |
| scar9::NifK | 60 | 10 |
| scar9::NifN | 46 | 9 |
| scar9::NifE | 42 | 1 |
| scar9::NifS | 41 | 1 |
| scar9::NifZ | 37 | 18 |
| scar9::NifM | 9 | 1 |
| ΔnifM | 6 | 4 |
| pB-ori (ΔNifHDKENJ) | 0 | 0 |

### Example 5. Detection of Scar9-Nif Fusion Polypeptides

To detect specific fusion polypeptides expressed in the bacterial system, liquid chromatography-mass spectrometry (LC-MS) methods were adopted. The method combined the physical separation capabilities of liquid chromatography with the mass analysis capabilities of mass spectrometry (MS) to detect specific peptides produced by digestion of protein extracts with trypsin.

E. coli strain JM109 separately containing each of the modified pMIT2.1 vectors together with pN249 were cultured and proteins extracted as described in Example 1. Protein samples were stored at ˜20° C. prior to reduction, alkylation and tryptic digestion. Protein samples were reduced, alkylated and treated with trypsin using a filter-assisted sample preparation (FASP) protocol as described in Example 1, and analysed by LC-MS as described in Example 1. The samples that were tested are listed in Table 7. Each genetic construct for samples 5-19 encoded one modified Nif polypeptide with the other 15 Nif polypeptides being wild-type as for K. oxytoca. Samples 1˜4 did not have any polypeptides including the scar9.

TABLE 7
Genetic constructs used for fusion polypeptide detection by LC-MS

| Sample | Genetic construct | Modified Nif | Scar9 peptide |
|---|---|---|---|
| ID | in JM109 | description | detected? |
| 1 | pMIT2.1 | positive control | |
| 2 | pB-ori | negative control | |
| 3 | pSO014 | ΔNifJ | |
| 4 | pSO051 | ΔNifM | |
| 5 | pSO006 | FAγ-Scar9-NifB | Yes |
| 6 | pSO026 | FAγ-Scar9-NifE | Yes |
| 7 | pSO028 | FAγ-Scar9-NifJ | Yes |
| 8 | pSO032 | FAγ-Scar9-NifF | Yes |
| 9 | pSO012 | FAγ-Scar9-NifH | Yes |
| 10 | pSO029 | FAγ-Scar9-NifK | Yes |
| 11 | pSO038 | FAγ-Scar9-NifM | Yes |
| 12 | pSO027 | FAγ-Scar9-NifN | Yes |
| 13 | pSO031 | FAγ-Scar9-NifQ | Yes |
| 14 | pSO034 | FAγ-Scar9-NifS | Yes |
| 15 | pSO033 | FAγ-Scar9-NifU | Yes |
| 16 | pSO035 | FAγ-Scar9-NifV | nd |
| 17 | pSO036 | FAγ-Scar9-NifW | Yes |
| 18 | pSO030 | FAγ-Scar9-NifY | Yes |
| 19 | pSO037 | FAγ-Scar9-NifZ | Yes |

nd: not done

Initially, 4 samples were assessed for trypsin digestion efficiency. Samples 5 (NifB) and 6 (NifE) were digested with trypsin for two incubation times, 30 min and overnight (16-18 h). From each sample, 4 μL of the tryptic peptides were injected on the 6600 Triple TOF Mass Spectrometer using an Eksigent microLC (85 min). Data were processed using ProteinPilot against the species-specific UniProt Knowledgebase (UniProtKB) databases appended with the custom and contaminant databases: Uniprot-Swiss Prot E. coli+Custom database (Mit2Nif)+Common Repository of Adventitious Proteins; Mit2Nif+Mit2.1 Nif-Scar. These databases included all of the predicted peptides produced by digestion of the Nif proteins with trypsin. The protein samples 5 and 6 from the constructs encoding FAγ-Scar9-NifB and FAγ-Scar9-NifE were expected to contain 16 Nif proteins, 15 of them being wild-type and the sixteenth having the scar9 on NifB and NifE, respectively.

The shorter duration trypsin digest of 30 min yielded more protein/peptide identifications than the longer digest. The full panel of E. coli samples (#1-19) were then digested with trypsin for 1 h rather than the overnight digestion.

The peptide identifications for the N-terminal scar9 sequence were investigated. Limited IDA (6600TF LC-MS/MS) evidence was found for the fully cleaved MSTQVVR (SEQ ID NO:55) and the semi-tryptic MSTQVVRNR (SEQ ID NO:49) peptides, with low peptide identification confidence. Peptides having either an unmodified or oxidised methionine residue were also assessed using MRM. However, these peptides could not be confirmed in the test samples using either discovery 6600TF LC-MS/MS and ProteinPilot database searches or targeted MRM 6500 QTRAP LC-MS/MS.

The possibility was considered that the translation initiating methionine might have been cleaved off post-translationally in the bacteria, as an explanation for the low peptide identification confidence. When recombinant proteins are expressed in bacterial expression systems, it is thought that the initiating methionine is often cleaved off by methionine aminopeptidase (MAP) with an efficiency based on the size of the residue adjacent to the N-methionine (Hirel 1989, Xiao 2010). When the residue at position 2 was a Ser residue, as was the case for the FAγ-scar9-Nif polypeptides, it was estimated that the N-terminal Met was often cleaved off (84% efficiency).

Therefore, additional modified peptides were assessed from the Nif fusion polypeptides: STQVVR (+1, +2) (SEQ ID NO:50) and the semi-tryptic peptide STQVVRNR (+2, +3) (SEQ ID NO:56). The peptide STQVVR (SEQ ID NO:50) was short and had not been identified in the previous analyses, probably for three reasons. Firstly, it had a mass (688 Da) that would yield an m/z value (345.2, +2) lower than set in the standard LC-MS parameters (m/z range 350-2000), secondly it had low hydrophobicity and therefore might not have been retained on the column, and thirdly it was too short for database search algorithms to confidently match the sequence. Initially, samples #1-19 (Table 7) were pooled and run on the 6600TF LC-MS/MS under different conditions e.g. lowering the mass range from m/z 350 to 300 and expanding the monitored charge states to include +1 rather than only +2 to +5, and by defining inclusion lists which encapsulated the predicted target masses. None of these changes yielded a positive identification of STQVVR (SEQ ID NO:50) in either the spectral data or the database searches.

The tryptic peptide STQVVR (SEQ ID NO:50) and semi-tryptic peptide STQVVRNR (SEQ ID NO:56) were then assessed using Multiple Reaction Monitoring (MRM) on the 6500 QTRAP using 4 transitions with 2 charge states. This yielded a peak for STQVVR (SEQ ID NO:50) that was investigated by Enhanced Production Ion (EPI) scanning to acquire full scan MS/MS spectra for the target MRM. This confirmed the presence of the modified truncated N-terminal peptide lacking the N-terminal Met. Encouragingly, it was concluded that the specific FAγ-Scar9-Nif polypeptides could be detected by this method from complex protein mixtures.

The method was then used to compare the expression level of each of the different FAγ-Scar9-Nif polypeptides when expressed from the modified pMIT2.1 vectors in E. coli. A comprehensive MRM method with 230 transitions was developed to assess the samples from JM109 (Table 7). This included high responding peptides (4 transitions/peptide) identified for the following Nif proteins: B, D, E, F, H, K, M, N, Q, S, U, W, Y and Z. Control peptides from the FAγ-Scar9 and the chloramphenicol acetyltransferase protein (CAT) were also included. The amount of a peptide specific to CAT in each sample was measured in order to standardize Nif levels between the different constructs, as described in Example 1. Care was taken to use equal total protein amounts in each of the samples. The amount of the CAT-specific peptide detected was similar across all experimental samples, indicating that the amount of Nif polypeptides generated in different samples from the pN249/pMIT2.1 assay system could properly be compared. The amount of peptide STQVVR (SEQ ID NO:50) derived from FAγ-Scar9 was observed to be highest in samples 9 (FAγ-Scar9-NifH) and 11 (FAγ-Scar9-NifM), both of which were strongly expressed relative to the others, followed by samples 10 (FAγ-Scar9-NifK), 14 (FAγ-Scar9-NifS) and 15 (FAγ-Scar9-NifU). Lower amounts were detected in the other samples 5-19 with the possible exception of NifV. There was no STQVVR (SEQ ID NO:50) peptide present in the negative control samples 1-4, as expected for the absence of the MTP-FAγ-scar9 sequence.

The amount of the scar9::NifD, scar9::NifK, scar9::NifH, scar9::NifS and scar9::NifM polypeptides in the E. coli cells was measured using targeted multiple reaction monitoring mass spectrometry (MRM-MS) as described in Example 1. The measurements showed that the amount of specific peptides for the NifS fusion polypeptide were about the same across all of the samples. In contrast, the greatest difference was found for scar9::NifM, where the amount of the NifM fusion polypeptide was about 50-fold increased relative to the samples where the wild-type NifM was expressed. In similar manner but to a lesser extent, the scar9 peptide fused to NifH resulted in a 2-3 fold increase in NifH abundance compared to the amount of wild-type NifH in the other strains. In the control samples in which the NifM gene was deleted (ΔNifM), peptides specific to NifM were not detected, as expected. Likewise, peptides specific for NifD, NifK and NifH were not detected in samples from E. coli containing pB-ori where these genes were not present. These analyses also showed that the abundance of NifD and NifK was reasonably consistent across all the samples, with the notable exception that in the presence of scar9::NifY, the amounts of NifD and NifK were reduced to about 30% of the levels found in the strains having the wild-type NifY. This reduction in NifD and NifK levels was confirmed by Western blot analysis of extracts from the E. coli cells, using antibodies that bound to wild-type NifD or NifK polypeptides. The inventors concluded that the addition of the scar9 motif to the N-terminus of Nif polypeptides, representing the product of the MPP-mediated cleavage of the MTP-FAγ51 fusions, when expressed in E. coli could influence the level of accumulation of the polypeptides, while preserving at least some activity for nitrogenase function.

In these analyses, the NifH-specific peptides were increased about 2-3-fold relative to control cells when, and only when, scar9-NifH was produced in the cells. In contrast, NifS and NifE were examples of polypeptides that accumulated consistently across all pMIT2.1-derived vectors, where the level of 2 NifS-specific peptides, or 2 NifE-specific peptides, and the scar9 extension peptide fused to CAT only varied by approximately 20% across all samples. These results indicated that the N-terminal alterations to the NifH and NifM polypeptides significantly increased the abundance of these two proteins relative to all other Nif proteins and CAT.

These results and those summarised in Table 4 provided some insight to the performance of scar9-extensions on NifH, NifM and NifE in nitrogenase function as measured by the ARA. Although the scar9-NifH polypeptide abundance was increased about 2-3-fold in the bacteria containing pSO012, the scar9-NifH provided 110% activity in the ARA assay relative to the wild-type control. On the other hand, the scar9-NifM accumulated much more relative to the wild-type control, but the ARA assay yielded only approximately 10% activity relative to the control. This result suggested that these high levels of scar9-NifM polypeptide might have been acting as a negative regulator to ARA function.

The LC-MS method was also used to detect specific fusion polypeptides in plant cells (Example 12), showing its general applicability.

### Example 6. Expression of K. oxytoca MTP-NifD in Plant and Yeast Cells Results in Production of a Secondary Cleavage Product

A previous report from the inventors had shown that of all the 16 Nif polypeptides, the most difficult to produce in plant cells was NifD (Allen et al., 2017). They also reported that when a MTP-FAγ::NifD::HA fusion polypeptide, having a wild-type K. oxytoca NifD amino acid sequence, was produced in N. benthamiana cells, additional bands of lower molecular weight showed on the Western blots. The additional bands included an intense band of ˜48 kDa. These additional bands were suggested to correspond to degradation products of the NifD fusion polypeptide, the result of a secondary cleavage at a cryptic protease site, or possibly the product of alternative transcription or translation initiation signals.

### Effect of Changing Promoters and MTP Sequences

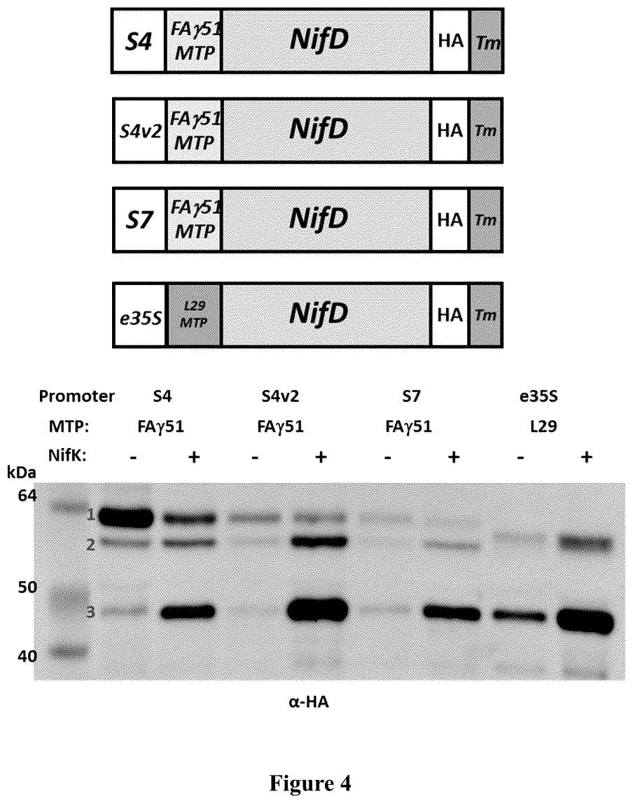

To confirm these observations and to test whether the additional bands were due to the combination of certain promoters or MTP sequences with the NifD sequence, a series of genetic modifications were made to the construct SN10. The starting construct SN10 encoded a MTP-FAγ51::NifD::HA fusion polypeptide (SEQ ID NO:122) where the NifD amino acid sequence was as set forth in SEQ ID NO:18, expressed from the enhanced e35S promoter and using codon optimisation for N. benthamiana. In some of the modifications, the e35S promoter of SN10 was substituted with a different promoter, for example with the S₄, S₄v2 or S₇ promoters of Subterranean Clover Stunt Virus (SCSV). In others, the MTP-FAγ51 was substituted with another MTP, for example MTP-L29 (SEQ ID NO:34) or MTP-CPN60 (SEQ ID NO:28). The constructs used in this experiment are listed in Table 8 and included some of those described in Example 2. These constructs were made via the GoldenGate cloning system (Weber et al., 2011) with specific components as described by Engler (2014). Some of the chimeric genes are shown schematically in FIG. 4 (upper panel).

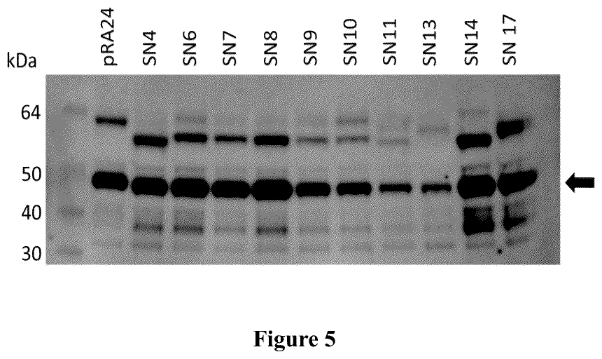

These constructs in A. tumefaciens were infiltrated into N. benthamiana leaf cells as described in Example 1 and protein extracts were analysed by Western blotting using HA-antibody. For each of the constructs, paired infiltrations were carried out either in the absence of the construct pRA25 or in the presence of pRA25 (encoding the MTP-FAγ::NifK fusion polypeptide; SEQ ID NO:57), since co-expression of NifK without a C-terminal extension had been shown to enhance NifD abundance (WO2018/141030). Representative Western blots are shown in FIGS. 4 and 5. It was observed that both the MPP-processed and unprocessed forms of the fusion polypeptide were produced for each construct, as was the ˜48 kDa polypeptide. In every case where pRA25 was present (FIG. 4, lower panel), the intensity of the ˜48 kDa band was greater than the intensity of the processed MTP::NifD polypeptide (band 2). This was also observed for all of the variants using a different MTP sequence; the 48 kDa polypeptide was the most intense of the polypeptide bands on the Western blots, irrespective of the MTP sequence used (FIG. 5). It was also observed that, once again, the presence of the MTP-NifK expression construct often increased the amount of all NifD polypeptides, including the dominant band at approximately 48 kDa.

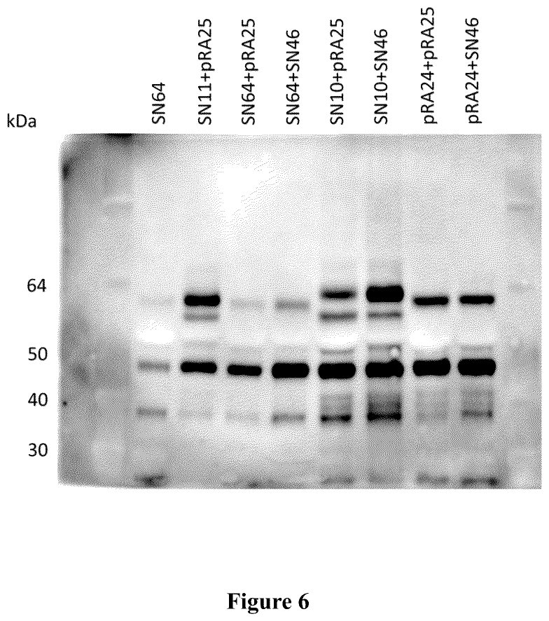

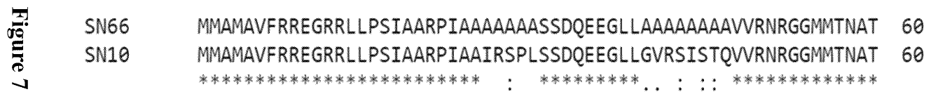

A different construct encoding a NifK fusion polypeptide, SN46, was made. This construct had the enhanced e35S promoter and a 5′-UTR including the TMV omega fragment to maximise translation efficiency, a 35S polyadenylation/transcription termination sequence, and encoded a MTP-Su9::NifK polypeptide having the wild-type C-terminus (SEQ ID NO:58). The coding region used codon optimisation for N. benthamiana rather than the human codon optimisation in pRA25. The SN46 construct was compared to pRA25 for effectiveness in increasing NifD fusion polypeptide accumulation after co-infiltration with a NifD construct. It was observed that SN46 was at least as effective as pRA25 in enhancing NifD fusion polypeptide accumulation, but also resulted in accumulation of the ˜48 kDa polypeptide product. A representative Western blot is shown in FIG. 6.

Since the ˜48 kDa polypeptide was detected using the HA-antibody, it corresponded to the C-terminal product of a protease cleavage of the translated fusion polypeptide. These results indicated that the ˜48 kDa C-terminal polypeptide was produced in plant cells from the wild-type K. oxytoca NifD fusion polypeptide irrespective of the promoter or MTP sequence used for its expression. The ˜48 kDa polypeptide is referred to herein as the NifD “secondary cleavage product” or as the NifD “degradation product”.

TABLE 8
Genetic constructs for testing production and processing of MTP-
NifD fusion polypeptides in plant cells. Each construct encoded
a fusion polypeptide including an MTP as listed fused to the N-
terminus of the wild-type K. oxytoca NifD sequence.

| Construct | | | |
|---|---|---|---|
| ID | Promoter | MTP | SEQ ID NO of MTP |
| SN4 | e35S | CPN60 No GG linker | SEQ ID NO: 29 |
| SN6 | SCSV-S4 | FAγ51 | SEQ ID NO: 21 |
| SN7 | SCSV-S4v2 | FAγ51 | SEQ ID NO: 21 |
| SN8 | SCSV-S7 | FAγ51 | SEQ ID NO: 21 |
| SN9 | 35S | FAγ51 | SEQ ID NO: 21 |
| SN10 | e35S | FAγ51 | SEQ ID NO: 21 |
| SN11 | e35S | CPN60 | SEQ ID NO: 28 |
| SN12 | e35S | SOD | SEQ ID NO: 30 |
| SN13 | e35S | 2SOD | SEQ ID NO: 31 |
| SN14 | e35S | SU9 | SEQ ID NO: 35 |
| SN15 | e35S | SODmod | SEQ ID NO: 32 |
| SN16 | e35S | 2SODmod | SEQ ID NO: 33 |
| SN17 | e35S | L29 | SEQ ID NO: 34 |
| SN19 | e35S | CoxIV twin strep | SEQ ID NO: 37 |

### Is the Secondary Cleavage Due to Mitochondrial Targeting?

The inventors aimed to determine the cause of NifD secondary cleavage/degradation, firstly whether it was occurring before or after mitochondrial import. To test this, a NifD construct (SN34) was made which was identical to SN10 except that the MTP-FAγ51 sequence was replaced with a HA epitope tag, so encoding a HA::NifD::HA fusion polypeptide. That polypeptide, lacking a MTP, would not be targeted to mitochondria but instead was expected to be localised in the cytoplasm of the plant cells. Having an HA epitope at both ends of the translation product, any internal protease cleavage was expected to produce a N-terminal product and a C-terminal product that could both be detected with the HA-antibody if they were not further degraded. A second genetic construct was made where the C-terminal HA tag was removed from SN34. That construct (SN33) encoded a HA:NifD fusion polypeptide which was almost identical in size to the MPP-processed MTP-FAγ51::NifD polypeptide, each possessing only one HA epitope tag, and so making the comparison more direct.

After co-infiltration of SN75 and SN46 into N. benthamiana and Western blot analysis of protein extracts from the infiltrated leaf cells, it was observed that SN33 and SN34 both produced discrete, strong bands corresponding in size to the full-length fusion polypeptides translated from these constructs. The main polypeptide band for SN34 was slightly larger than the polypeptide band for SN33, understood to be due to the presence of the additional C-terminal HA epitope in SN34. These SN33 and SN34 NifD specific bands were considerably stronger in intensity than the corresponding full-length band produced from cells infiltrated with SN10. Importantly, there was no 48 kDa C-terminal cleavage/degradation product observed after introduction of SN34 and SN33. Similarly, there was no N-terminal cleavage product observed for SN34.

A further construct designated SN66 was made which had a mutated MTP sequence in order to test whether production of the 48 kDa polypeptide required a first cleavage in the MTP sequence by MPP. For this, the MTP-FAγ51 encoded in SN10 was modified with a sequence of identical length that contained a region of 5 consecutive alanine substitutions in the MTP and a second region of 8 substitutions that would render it resistant to mitochondrial processing by MPP. The specific substitutions are shown in FIG. 7. The second alanine scanned region encompassed the recognition and cleavage site for MPP and therefore MPP-processing was predicted be abolished due to these substitutions. It was not known if this fusion polypeptide would be transported to the mitochondria. When this construct was introduced into N. benthamiana leaf cells, protein extracts from the cells were observed to contain the 48 kDa product by Western blot analysis.

A second construct designated SN64 was made having a similarly mutated MTP sequence having alanine substitutions compared to the MTP-CPN60 sequence (SEQ ID NO:28). When this construct was tested in N. benthamiana leaf cells, the 48 kDa secondary cleavage product was again observed (FIG. 6).

Together these results demonstrated that the secondary cleavage/degradation of the MTP::NifD fusion polypeptides was a consequence of the mitochondrial targeting and was presumed to be caused by a mitochondrial protease. However, the secondary cleavage was not dependent on a prior cleavage of the MTP sequence by MPP in the mitochondria.

Detection of an N-Terminal NifD Cleavage Product Demonstrated that Secondary Cleavage was at a Specific Site by an Endoprotease

As the 48 kDa C-terminal cleavage/degradation product was clearly produced in the plant cells after introduction of SN10 and other constructs encoding the MTP::NifD fusion polypeptides, the inventors wanted to see whether a corresponding N-terminal NifD cleavage product could be observed in the plant cells or whether the degradation occurred by exo-protease activity from the N-terminus. Accordingly, another construct (SN75) was made which was identical to SN10 except that a Gly-Gly linked HA tag was also included directly after the MTP-FAγ51 and before the NifD coding region, and SEQ ID NO:36 was used as the MTP-FAγ51. It was predicted that if the fusion polypeptide produced from this construct was cleaved at the same specific location within NifD, two HA-tagged products would be produced—the longer ˜48 kDa C-terminal product seen previously in MTP::NifD extracts and a shorter ˜13 kDa N-terminal product. However, given that a specific peptidase in mitochondria degrades N-terminal cleaved presequences after MPP cleavage (Kmiec et al, 2013), the inventors did not know whether any N-terminal cleavage/degradation product would be observed.

After infiltration of SN75 into N. benthamiana leaves and Western blotting analysis of protein extracts, a shorter N-terminal product of approximately 15 kDa was detected as well as the longer C-terminal product of approximately 48 kDa. Although the sum of the sizes of these two products was slightly greater than the predicted size of the MPP-processed MTP-FAγ51::HA::NifD::HA polypeptide (57.6 kDa), this difference was likely a result of overestimating band sizes relative to the markers which may have been due to the surface charges of the polypeptides affecting the migration rate in the gel electrophoresis. Nevertheless, this result demonstrated that the secondary cleavage of the NifD part of the fusion polypeptide was specific and discrete, occurring at a specific site in the NifD polypeptide, and not a result of sequential degradation from the N-terminus.

### Does the Secondary Cleavage/Degradation of Mitochondrially-Targeted NifD Occur in Yeast?

Buren et al. (2017b) reported that targeting an Azotobacter vinelandii NifD polypeptide to yeast mitochondria produced a faster migrating ˜50 kDa band detectable by NifD antibodies. The present inventors wanted to determine if the plant-optimised K. oxytoca NifD sequences also exhibited a similar cleavage when expressed in yeast. For this purpose, a yeast expression vector was made which included the MTP-FAγ51::NifD::HA coding sequence from SN10 with flanking KpnI/SacI restriction sites to allow cloning into the yeast expression vector pYES2. This construct was designated SNY10. As a control for non-mitochondrial localisation, a second yeast NifD construct designated SNY196 was made where the MTP-FAγ51 of SNY10 was replaced with a 6×His epitope tag. This second construct was designed to express a cytoplasmically-localised NifD polypeptide of almost the same size as the processed polypeptides from SN10 or SNY10, thereby enabling visualisation of the expected size on the Western blots. A plant orthologue of SNY196 was also made (SN196) where the GALL promoter was replaced with the e35S promoter. This construct was identical to SN10 except the 6×His tag substituted for the MTP-FAγ51 of SN10.

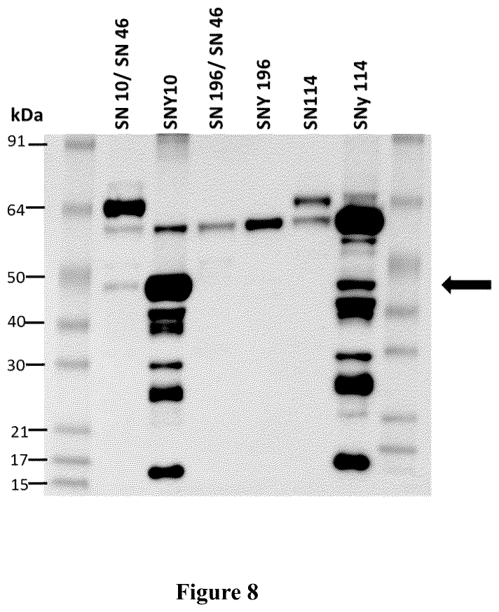

Yeast cells containing either the SNY10 (MTP-FAγ51::NifD::HA) or the SNY196 (6×His::NifD::HA) constructs were grown as described in Example 1 for expression of the genes encoding the fusion polypeptides. Proteins were extracted from the transformed cells after induction of transgene expression and analysed by Western blotting with HA-antibody. The results are shown in FIG. 8. In the lane for SNY10, a less intense band was observed of the size expected (˜58 kDa) for a MPP-processed MTP-FAγ51::NifD::HA polypeptide. This polypeptide was of the same size as the plant expressed MTP-FAγ51::NifD::HA polypeptide after MPP processing and the polypeptide from SN196. Importantly, a much more intense polypeptide band at ˜48 kDa band was observed from SNY10 which was of the same size as the plant expressed cleavage/degradation product from SN10. That is, most of the yeast expressed MTP-FAγ51::NifD::HA was cleaved in a similar fashion to the cleavage in plant cells, indeed even more efficiently in the yeast cells. The fact that the C-terminal cleavage products from the yeast and plant cells were of the same size indicated that the protease cleavage was occurring at the same site in both yeast and plant cell mitochondria. In contrast, the protein extract from yeast cells containing SNY196 produced a single, discrete band of the expected size for non-mitochondrially targeted NifD. No specific, C-terminal NifD::HA polypeptide band was detected from SNY196 at ˜48 kDa that would have indicated non-mitochondrial cleavage at the same site.

Remarkably, no MTP::NifD fusion polypeptide that was not processed by MPP was detected in protein extracts from the yeast cells containing SNY10, in contrast to the observation that in N. benthamiana cells producing the same MTP-FAγ51::NifD polypeptide, both unprocessed and MPP-processed forms of the polypeptide were observed. That is, in yeast the MTP sequence was fully processed by MPP. This was thought to reflect differences in processing machinery and efficiencies between the two organisms. It might also have come from the fact that the yeast cells were stably transformed cells in contrast to the plant cells which were only transiently transformed.

Together these results indicated that the wild-type NifD polypeptide from K. oxytoca, expressed as an MTP-fusion polypeptide, was cleaved at the same specific site when targeted to yeast or plant mitochondria and that the cleavage was dependent on mitochondrial targeting.

### Example 7. Identification of the Secondary Cleavage Site in Wild-Type NifD

The results of the experiments described in Example 6 indicated that the secondary cleavage of the MTP::NifD fusion polypeptide occurred at a specific site within the wild-type NifD sequence and was a consequence of mitochondrial targeting. As the cleavage was considered to be undesirable for several reasons, the inventors wanted to modify the region of NifD in an attempt to prevent the cleavage in plant cells. From the sizes of the N-terminal and C-terminal cleavage products, the cleavage site was thought to lay in the region of amino acids 80-120 of the wild-type NifD sequence (SEQ ID NO:18). However, the possibility existed that cleavage at the specific site was influenced by distal sequences, not just the amino acids adjacent to the cleavage site. For this reason, the inventors took a broader approach to identifying the specific site of secondary cleavage and the surrounding amino acids and possible further regions which could influence the cleavage.

As an initial attempt to identify the cleavage site within NifD or at least predict its location, both the unprocessed and MPP-processed amino acid sequences were entered into Mitofate software (Fukusawa et al., 2015) to see whether any MPP sites were predicted. The Mitofate software predicts sites for cleavage by MPP by incorporating amino acid sequence features including positively charged amphiphilicity and presequence motifs as well as amino acid composition and physico-chemical properties. The software also predicts presequence cleavage sites by MPP by generation of a consensus position weight matrix between amino acid residues −4 and +5 of aligned cleavage sites of a yeast training data set. This tool also incorporates information on the distance from the N-terminus, as MTPs are generally between 10-90aa long, with a minority being longer than 110aa (Huang et al., 2009).

Assuming that MPP might recognise the secondary cleavage site after an initial cleavage within the MTP as the preprotein travelled through the outer and inner mitochondrial membranes, the amino acid sequences resulting from the initial MPP processing event were entered into the Mitofates software for two lengths of the MTP-FAγ, namely FAγ-scar37-NifD (35aa FAγ scar plus GG) and FAγ-scar11-nifD (9aa FAγ51 scar plus GG). The analysis by Mitofates using the sequence FAγ-scar37-NifD returned a predicted cleavage site immediately after amino acid G62 within the sequence VRGCAY (SEQ ID NO:60) relative to the N-terminus of NifD, and the sequence FAγ-scant 1-NifD returned a predicted cleavage site immediately after N₉₉ in the sequence RAGRRNYYTG (SEQ ID NO:61). The Mitofate analysis therefore showed that the NifD sequence in this area appeared to possess characteristics of one or even two MPP processing sites. As described below, the second of these predicted sites turned out to be correct for the secondary cleavage.

In a different approach to identify regions in NifD that were involved in the secondary cleavage, a series of genetic constructs were made each with a block of 5 consecutive amino acid substitutions within the approximate region of secondary cleavage of NifD, where non-alanine amino acids were replaced with alanines and native alanine amino acids were replaced with glycines. That is, alanine was used for all substitutions except that the native alanine residues were replaced with glycine. The series of substitution mutants spanned about 6 kDa of the presumptive cleavage site from amino acid 49 to amino acid 108 of SEQ ID NO:18. These constructs were designated NifD-Var 1 to 6 and Var 9 to 14 (Table 9). Two other variants were made with discrete substitutions based on the Mitofates prediction of a possible cleavage site within the sequence VRGCAY (SEQ ID NO:60), designated NifD-Var 7 and Var 8. In all other respects these constructs encoding NifD variants were identical to SN10 in that the polypeptides had the MTP-FAγ51 translationally fused to a NifD protein coding region and a C-terminal HA epitope tag that would allow detection of any NifD C-terminal cleavage product.

These 14 constructs were introduced from A. tumefaciens individually into N. benthamiana leaf cells together with SN46 (MTP-Su9::NifK). Protein extracts were prepared from infiltrated leaf spots and subjected to SDS-PAGE and Western blotting using HA-antibody. Of the 14 variants tested, 12 still produced the 48 kDa cleavage product and were indistinguishable in their banding pattern compared to the bands derived from SN10 having the wild-type NifD sequence. However, NifD-Var 13 (genetic construct SN100) was conspicuous in showing no 48 kDa cleavage product and, from the size and intensity of the band on the Western blot, a relatively higher ratio of processed to unprocessed FAγ51::NifD than the other variants. For NifD-Var 12 (SN99), a faint band was detected at 48 kDa, considerably less in intensity than for the wild-type. Again, the ratio of MPP-processed NifD to unprocessed NifD was greater for NifD-Var 12 compared to the wild-type and the variants other than NifD-Var13. Based on the amino acids substituted in NifD-Var 12 and 13, it was concluded that a specific region of the NifD polypeptide including at least some amino acids within the amino acid sequence RAGRRNYYTG (SEQ ID NO:61) corresponding to amino acids 94-103 of SEQ ID NO:18 was required for the secondary cleavage of NifD in mitochondria.

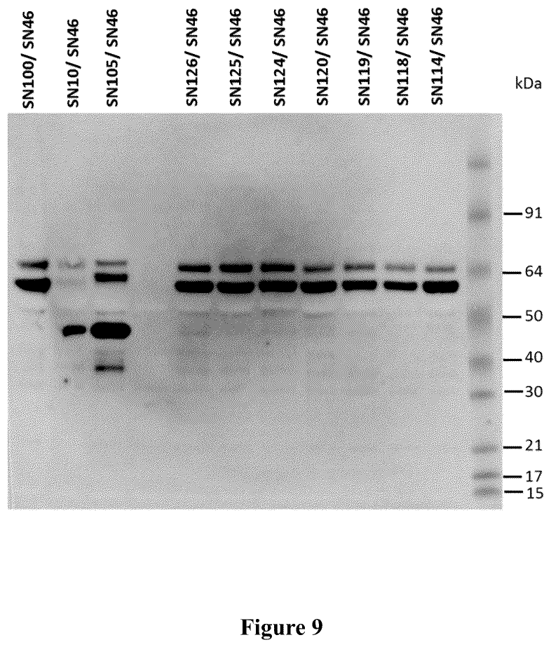

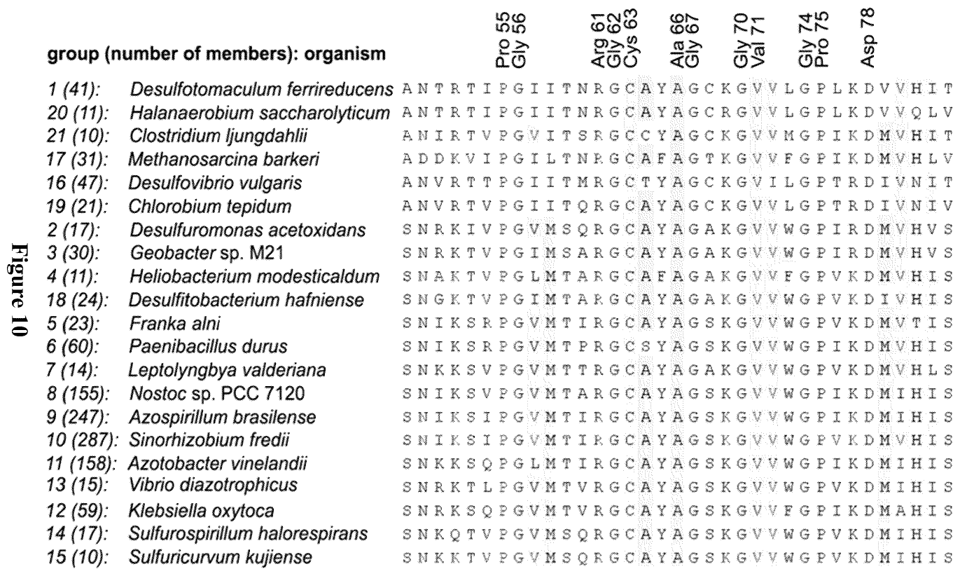

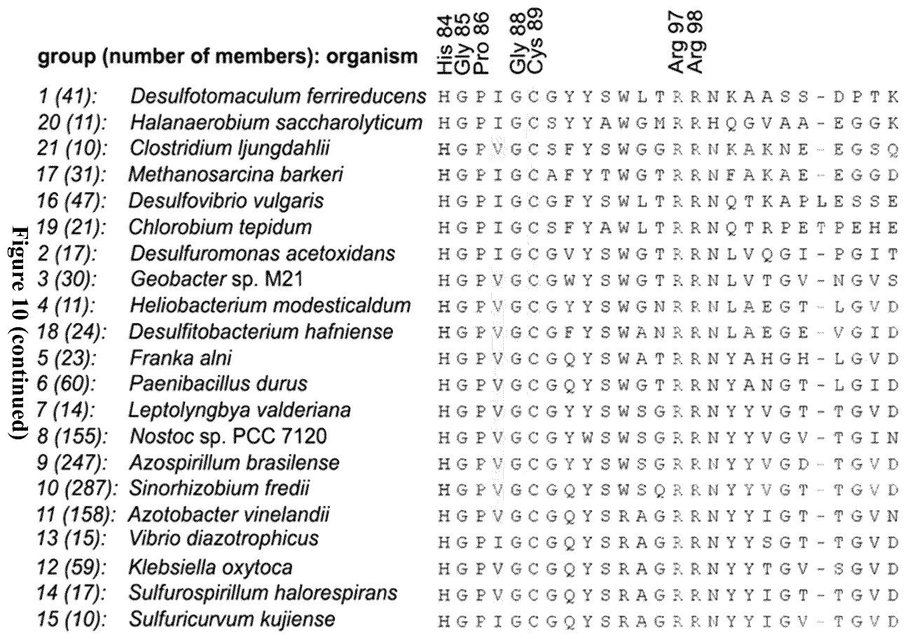

Based on that experiment and the conclusion drawn, genetic constructs encoding a second set of amino acid variants of NifD were made in which one, two or three of the amino acids within the RAGRRNYYTG (SEQ ID NO:61) sequence were substituted. In this set of variants, alanines were not used instead of the wild-type amino acids but rather changes based on phylogenetic analysis of a large set of naturally occurring NifD sequences (see below) and modelling of NifD-NifK structures was used to identify replacement amino acids at each particular position. The concept here was that naturally occurring variants of the RAGRRNYYTG (SEQ ID NO:61) sequence might be more likely to maintain NifD function and that rational design of variations was possible to avoid secondary cleavage and maintain function. Each construct was identical to SN10 except for the amino acid substitution(s), so encoding a polypeptide having the MTP-FAγ51 fused to NifD and then a C-terminal HA epitope tag to enable detection of a 48 kDa C-terminal cleavage product. The substitutions in this set of NifD variants, designated NifD-Var 15 to 36, are listed in Table 10 and a representative Western blot is shown in FIG. 9.

The 19 individual genetic constructs (SN108-SN126), each encoding one of the variant NifD sequences, were introduced into N. benthamiana cells via A. tumefaciens and, after 5 days for expression of the chimeric genes, proteins were extracted and subjected to SDS-PAGE and Western blotting using HA-antibody. As was done previously, the genetic construct SN46 encoding MTP-Su9::NifK was co-infiltrated with each NifD variant in order to increase the level of NifD accumulation. From the Western blot data, three groups of variants were observed: (1) Those that showed an identical banding pattern to that obtained with SN10, comprising the wild-type NifD sequence, namely SN108, SN109, SN111-113, SN115, SN116 and SN121. For these, the ratio of the intensities of the 48 kDa band to the MPP-processed NifD (primary cleavage) was essentially the same as for SN10, indicating that the secondary cleavage was not affected by the amino acid substitution(s). (2) Those that showed a 48 kDa product, but the ratio of the intensities of the 48 kDa product to the MPP-processed NifD was noticeably reduced compared to the ratio for SN10 (SN110, SN122 and SN123). (3) Variants that showed no 48 kDa secondary cleavage/degradation product (SN114, SN117, SN118, SN119, SN120, SN124, SN125 and SN126), that is, the secondary cleavage was eliminated or reduced to the extent that it was not detected, by 1-3 specific amino acid substitutions. Most remarkably, two of this last set, namely NifD-Var 21 having a Y100Q substitution (encoded by SN114) and NifD-Var29 having a Y100K substitution (SN119) had single amino acid substitutions, and another variant Var 24 encoded by SN117 had two amino acid substitutions YY100-101QT. That these specific amino acid substitutions would have had this effect could not possibly have been predicted beforehand.

From this set of variants, it appeared that substitution of the arginine at position 98 alone did not prevent the secondary cleavage (NifD-Var 19 and Var 32). Likewise, single amino acid substitutions of the asparagine at position 99 (NifD-Var20), the tyrosine at position 101 (NifD-Var 15 and Var 22), the threonine at position 102 (NifD-Var 16 and Var 23) or 2 or 3 substitutions at positions 101-103 alone did not prevent the secondary cleavage. However, the single, double or triple substitutions that were tested including the tyrosine at position 100 (NifD-Var 21, 24, 26, 29 and 30) all abolished the secondary cleavage of NifD. Cleavage was also abolished by double or triple substitutions of amino acids not including the tyrosine at position 100 (NifD-Var 34, 35 and 36). It was clear that multiple variants could be readily identified having amino acid substitutions at positions selected from amino acid positions 98-102 which were resistant to the secondary cleavage, for example through using the approach exemplified here.

### Abolition of Secondary Cleavage of MTP::NifD in Yeast

Given the data in Example 6 that cleavage of the MTP::NifD fusion polypeptide occurred at the same region in yeast cells as in plant mitochondria, the variant having the Y100Q substitution was tested in yeast mitochondria. For this purpose, the protein coding region from SN114 (MTP-FAγ51::NifD(Y100Q)::HA) was amplified by PCR to provide flanking KpnI and Sad restriction enzyme sites and these were used to insert the gene into the yeast expression vector pYES2. This construct for yeast expression was designated SNY114. Protein extracts were obtained from yeast transformants containing SNY114 and analysed by Western blotting. Remarkably, the extracts from cells containing SNY114 produced a strong band at the same size as the NifD-Var 29 construct in plant cells, with a much reduced amount of secondary cleavage occurring. This contrasted strongly with the result in FIG. 8 with the wild-type NifD sequence which when expressed in yeast produced an intense 48 kDa cleavage/degradation product. Although there were some protein bands of other sizes observed from SNY114, these were less intense than the predominant full length band corresponding to the desired MPP-processed MTP::NifD::HA polypeptide. It was concluded that the full length, correctly processed NifD polypeptide was expressed as the predominant MTP::NifD polypeptide in yeast mitochondria, as in plant mitochondria, when amino acid substitutions were included at positions 98-102 of NifD with reference to SEQ ID NO:18, for example at position 100.

TABLE 9
Alanine-substitution variants of FAγ51-NifD fusion polypeptide and effect on secondary
cleavage/degradation in plant cells.

| | Con- | | | Position | Secondary |
|---|---|---|---|---|---|
| Vari- | struct | | | in SEQ ID | cleavage |
| ant | No | Wild-type sequence | Modified sequence | NO: 18 | efficiency |
| 1 | SN52 | SNRKS (SEQ ID NO: 62) | AAAAA (SEQ ID NO: 63) | 49-53 | ++ |
| 2 | SN53 | QPGVM (SEQ ID NO: 64) | AAAAA (SEQ ID NO: 63) | 54-58 | ++ |
| 3 | SN54 | TVRGC (SEQ ID NO: 65) | AAAAA (SEQ ID NO: 63) | 59-63 | ++ |
| 4 | SN55 | AYAGS (SEQ ID NO: 66) | GAGAA (SEQ ID NO: 67) | 64-68 | ++ |
| 5 | SN56 | KGVVF (SEQ ID NO: 68) | AAAAA (SEQ ID NO: 63) | 69-73 | ++ |
| 6 | SN57 | GPIKD (SEQ ID NO: 69) | AAAAA (SEQ ID NO: 63) | 74-78 | ++ |
| 7 | SN58 | TVRGCAYAGS (SEQ ID NO: 70) | TARACGYGGS (SEQ ID NO: 71) | 59-68 | ++ |
| 8 | SN59 | AYAG (SEQ ID NO: 72) | GAGG (SEQ ID NO: 73) | 64-67 | ++ |
| 9 | SN96 | MAHIS (SEQ ID NO: 74) | AGAAA (SEQ ID NO: 75) | 79-83 | ++ |
| 10 | SN97 | HGPVG (SEQ ID NO: 76) | AAAAA (SEQ ID NO: 63) | 84-88 | ++ |
| 11 | SN98 | CGQYS (SEQ ID NO: 77) | AAAAA (SEQ ID NO: 63) | 89-93 | ++ |
| 12 | SN99 | RAGRR (SEQ ID NO: 78) | AGAAA (SEQ ID NO: 75) | 94-98 | + |
| 13 | SN100 | NYYTG (SEQ ID NO: 79) | AAAAA (SEQ ID NO: 63) | 99-103 | − |
| 14 | SN101 | VSGVD (SEQ ID NO: 80) | AAAAA (SEQ ID NO: 63) | 104-108 | ++ |

TABLE 10
Second set of amino acid substitution variants of FAγ51-NifD fusion
polypeptide and effect on secondary cleavage/degradation and impact on function
in ARA bacterial assays. The wild-type sequence RAGRRNYYTG (SEQ ID NO: 61)
was replaced with the indicated modified sequence. Nt = not tested.

| | | | Secondary | |
|---|---|---|---|---|
| | Construct | | cleavage | NifD |
| Variant | No. | Modified sequence | efficiency | function |
| 15 | SN108 | RAGRRNYFTG (SEQ ID NO: 81) | ++ | Nt |
| 16 | SN109 | RAGRRNYYAG (SEQ ID NO: 82) | ++ | Nt |
| 17 | SN110 | RAGRRNYFAG (SEQ ID NO: 83) | + | Nt |
| 18 | SN111 | RAGRRNYYAA (SEQ ID NO: 84) | ++ | Nt |
| 19 | SN112 | RAGRANYYTG (SEQ ID NO: 85) | ++ | Nt |
| 20 | SN113 | RAGRRHYYTG (SEQ ID NO: 86) | ++ | Nt |
| 21 | SN114 | RAGRRNQYTG (SEQ ID NO: 87) | − | 147% |
| 22 | SN115 | RAGRRNYTTG (SEQ ID NO: 88) | ++ | Nt |
| 23 | SN116 | RAGRRNYYVG (SEQ ID NO: 89) | ++ | Nt |
| 24 | SN117 | RAGRRNQTTG (SEQ ID NO: 90) | − | 107% |
| 26 | SN118 | RAGRRHKGTG (SEQ ID NO: 91) | − | 33% |
| 29 | SN119 | RAGRRNKYTG (SEQ ID NO: 92) | − | 94% |
| 30 | SN120 | RAGRRNKATG (SEQ ID NO: 93) | − | 67% |
| 31 | SN121 | RAGRRNYATG (SEQ ID NO: 94) | ++ | Nt |
| 32 | SN122 | RAGRKNYYTG (SEQ ID NO: 95) | + | Nt |
| 33 | SN123 | RAGRKNYFTG (SEQ ID NO: 96) | + | 4% |
| 34 | SN124 | RAGRKNYYAG (SEQ ID NO: 97) | − | 1% |
| 35 | SN125 | RAGRKNYFAG (SEQ ID NO: 98) | − | 2% |
| 36 | SN126 | RAGRKNYAAG (SEQ ID NO: 99) | − | Nt |

### Demonstration of the Secondary Cleavage Site by Mass Spectrometry

Protein extracts from N. benthamiana leaves infiltrated with SN14 (MTP-Su9::NifD::HA) were run on SDS-PAGE using a gel having a polyacrylamide concentration of 4-20% (Invitrogen). The gel was stained with Aqua stain (Bulldog Bio). After destaining in water, 5 slices were cut from the gel for the region spanning the molecular weights 37-50 kDa. The slices were numbered 1 to 5 from the smaller molecular weights to the larger. Each gel slice was cut into approximately 1 mm cubes and soaked in 150 μL 30% methanol for 15 minutes. To reduce proteins that may have oxidised, the buffer was removed and replaced with 100 μL of fresh 25 mM ammonium bicarbonate (ABC) buffer with 5 μL of 15% dithiothreitol and incubated at room temperature for an hour. Cysteine residues were inactivated by the addition of 5 μL of 40% acrylamide and incubation at room temperature for 1 hour, after which the buffers were carefully removed. Three wash steps were carried out, each of 50 μL of ABC buffer and 50 μL acetonitrile and incubation at room temperature. The gel pieces were dried by the addition of 100 μl of 100% acetonitrile for 2 min, which was then discarded. The proteins in the dried gel pieces were then digested with 0.1 μg trypsin (Promega) in 20 μl ABC with incubation overnight at 37° C. The tryptic digest was stopped with 1 μL of a 50% (v/v) formic acid solution and sonication for 15 min. The samples were filtered after the addition of 10 μL of water before transfer into LCMS vials.

The resulting tryptic digest from each gel slice was injected onto a Dionex Nanomate 3000 (ThermoFisher) nano liquid chromatography (LC) system directly coupled to an Orbitrap Fusion Tribrid Mass Spectrometer. The peptides were desalted for 5 min on an Acclaim PepMap C18 (300 Å, 5 mm×300 μm) trap column at a flow rate of 10 μL/min with loading solvent, and separated on an Acclaim PepMap C18 (100 Å, 150 mm×0.075 mm) column at a flow rate of 0.3 μL/min at 35C. A linear gradient from 5% to 40% solvent B over 60 min was employed followed by a wash and re-equilibration with 40-99% B over 5 min, a 5 min hold at 99% B, return to 5% B over 6 min, and held for 7 min. The solvents used were: (A) 0.1% formic acid, 99.9% water; (B) 0.08% formic acid, 80% acetonitrile, 19.92% water. The nano-LC was directly coupled to the Nanospray Flex Ion source of the Orbitrap Fusion MS. The ion spray voltage was set to 2400 V, the sweep gas was set to 1 Arb and the ion transfer tube temperature was set to 300° C. Data were acquired in data-dependent acquisition mode consisting of a Orbitap-MS survey scan followed by parallel acquisition of a high resolution Orbitrap scan at 120,000 resolution and multiple MS/MS events in the linear ion trap, over a 3 second period. First stage MS analysis was performed in positive ion mode over the mass range of m/z 400-1500 with an AGC target of 4×10 5 and a maximum injection time of 50 ms. Tandem mass spectra were acquired in the ion trap on precursor ions that exceeded an intensity threshold of 1000 counts with charge state 2-7. Spectra were acquired using quadrupole isolation with a 1.6 m/z isolation window and (Higher energy Collisional Dissociation) HCD set at 28% based on the size and charge of the precursor ion for optimum peptide fragmentation. Ion trap scan rate was set to rapid with an AGC target of 4×10 3 and a maximum injection time of 300 ms, the instrument was set to utilise the maximum parallelizable time for injecting ions into the trap during a 3 second window whilst the orbitrap was collecting high resolution MS spectra. Dynamic exclusion was set to exclude precursor ions after one occurrence with a 15 sec interval and a mass tolerance of 10 ppm.

Analysis of the data for protein identification was conducted using the Sequest algorithm in Proteome Discoverer v2.2 (ThermoFisher). Carbamidomethyl was selected as the alkylating agent and trypsin was selected as the digestion enzyme. Dynamic modifications were selected for oxidation on NifD with a maximum of three modifications. Tandem mass spectrometry data were searched against a database of tryptic peptides for NifD derived from the fusion polypeptide amino acid sequence encoded by SN14 and the N. benthamiana proteome, common contaminants and organism specific databases annotated from UniProt. The database search results were curated to yield the protein identifications using a 1% global false discovery rate (FDR) determined by the in-built FDR tool within Proteome Discoverer software.

Of the five samples submitted to mass spectrometry, no NifD peptides were identified in the sample coming from the gel slice for the highest molecular weight, sample 5. In contrast, NifD peptides were identified in the other samples 1-4. The greatest coverage was for sample 2, the second lowest band excised from the gel, with 17 specific tryptic peptides derived from the NifD sequence being identified in that sample. Six to 11 specific NifD peptides were identified in samples 1, 3 and 4. Importantly, the peptide YYTGVSGVDSFGTLNFTSDFQER (SEQ ID NO:100) was positively identified in sample 2. The XCorr score was sufficiently high for that peptide and the posterior error probability (PEP) score was sufficiently low to confirm the positive identification, indicating that the peptide fragment ions were not the product of a similarly sized but different peptide. It was concluded that this peptide must have arisen from SN14 by a specific cleavage of the NifD sequence within the RRNY sequence (SEQ ID NO:101) between the asparagine (N) and tyrosine (Y) residues in the plant cells, followed by the tryptic digestion in the analysis. The positive identification of the cleavage site by this MS analysis was in complete agreement with the mutational approach described above.

### Example 8. Phylogenetic Analysis of NifD Around the Secondary Cleavage Site

Nitrogenase enzymes including NifD polypeptides are naturally produced in numerous bacterial and archaeal phyla. A set of 1751 naturally occurring NifD amino acid sequences from a very wide range of bacterial and archaeal sources was extracted from the InterPro database on 12 Dec. 2018. All of the sequences were listed as members of the family IPR005972, defined as Nitrogenase molybdenum-iron protein alpha chain which are all NifD polypeptides of the molybdenum-iron type. The sequences were from 21 different phyla. The majority of sequences were from Proteobacteria (63.0%) followed by Firmicutes (12.3%) and Cyanobacteria (12.3%). Others at lower numbers were from the phyla Actinobacteria, Aquificae, Bacteroidetes, Candidatus Margulisbacteria, Candidatus Sumerlaeota, Chlorobi, Chloroflexi, Chordata, Chrysiogenetes, Deferribacteres, Elusimicrobia, Euryarchaeota, Fusobacteria, Lentisphaerae, Nitrospirae, Planctomycetes, Spirochaetes and Verrucomicrobia.

The set of 1751 sequences contained 275 duplicate sequences. The duplicate sequences were removed, which resulted in a set of 1476 unique sequences. These were examined to understand the diversity of amino acid sequences at positions corresponding to the RAGRRNYYTG sequence (SEQ ID NO:61) of K. oxytoca. The sequences were aligned using the multiple sequence alignment program Mafft version 7 using the FFT-NS-2 strategy with default parameters, i.e using the default “fast and progressive” settings (Katoh et al., 2013). The aligned sequences were visualised using the ALVIS software (interactive non-aggregative visualization and explorative analysis of multiple sequence alignments) (Schwarz et al., 2016). The NifD sequences were between 362 and 592 residues long. The multiple sequence alignment (‘mega-alignment’) contained 907 positions, taking into account the numerous gaps in the individual sequences that were introduced by the alignment program. In the mega-alignment, the proposed secondary cleavage site was found between positions 270 to 275, corresponding to residues 97 to 102 in the K. oxytoca sequence (SEQ ID NO:18). Sixty-eight sequences were identified that contained the same 10 amino acids sequence as amino acids 94-103 of K. oxytoca (RAGRRNYYTG; SEQ ID NO:61).

A protein similarity network was generated for the 1476 members of the InterPro family IPR005972, showing clusters of related sequences from different phyla of diazotrophs. Representative sequences were chosen from different clusters (Table 11) and aligned in the region corresponding to amino acids 49-108 of the K. oxytoca NifD sequence. The alignment for that region is shown in FIG. 10. A high degree of sequence conservation was noted, including 19 amino acids that were completely conserved and many others that were highly conserved. Not shown in FIG. 10, the sequences from Desulfotomaculum ferrireducens, Halanaerobium saccharolyticum, Clostridium ljungdahlii, Methanosarcina barkeri, Desulfovibrio vulgaris and Chlorobium tepidum and the related sequences in their clusters contained a 50-60 residue insertion further towards the C-terminus, therefore forming a subgroup of NifD sequences.

The frequency distribution of residues was calculated around the predicted secondary cleavage site, taken as being immediately after the RRN amino acids, for positions −3, −2, −1, +1, +2 and +3 (Table 12). The arginine (R) in position −3 was completely conserved except for two sequences in the set that showed a “gap” for both positions −3 and −2. However, these two sequences were only fragments, not complete NifD sequences (A0A2N4YT47 —Klebsiella variicola, A0A2N5A8Y2 —Klebsiella variicola) and therefore were uncertain and could be excluded from further analysis. The arginine at position −2 was almost completely conserved. There were only two sequences out of the 1476 that contained a residue other than arginine at that position: NifD from Paenibacillus fujiensis (B9X2A1) contained a cysteine residue and NifD from Alcaligenes faecalis (Q44045) contained a glycine residue. It was not known whether these sequences were active for NifD. Asparagine (N) was highly conserved at position −1, present in 97.83% of the 1476 sequences. About 1.9% of the 1476 sequences contained a histidine, phenylalanine, alanine or serine residue at that position instead of asparagine. The most frequent residue at position +1 was tyrosine (Y, 71.54%), followed by glutamine, leucine and lysine, each of those three at a frequency in the range of 7-11%. As there were substantial numbers of naturally occurring NifD sequences having one of these amino acids other than tyrosine at that position, it was concluded that those amino acids at position +1 provided for NifD activity. Phenylalanine, methionine and glutamic acid were also represented in that position at lesser frequencies. The most frequent residue at position +2 was tyrosine (64.43%), followed by alanine and threonine, and then any of six other amino acids at lower frequencies. Again, it was concluded that those amino acids at position +2 provided for NifD activity. The most frequent residue at position +3 was valine (V, 27.24%), followed by isoleucine, threonine and lysine, then any of 11 other amino acids. Clearly, the degree of conservation of the amino acids in the six positions corresponding to residues 97-102 of the K. oxytoca NifD sequence decreased along that sequence, from the two arginines which were considered to be essential to NifD function through to position +3 which showed wide variability.

The 1474 NifD amino acid sequences (excluding the two partial sequences mentioned above) were then examined at the positions corresponding to amino acids 97-101 of SEQ ID NO:18 for the presence of the sequence RRNY (SEQ ID NO:101) and more specifically within that set RRNYY (SEQ ID NO:102). There were 1045 sequences (70.90%) that comprised RRNY (SEQ ID NO:101) and, of those, 935 sequences comprising RRNYY (SEQ ID NO:102) corresponding to amino acids 97-101 of SEQ ID NO:18. On the basis of the secondary cleavage data described above, it was concluded that the 1045 naturally occurring NifD polypeptides having the sequence RRNY (SEQ ID NO:101) would be subject to secondary cleavage within that sequence upon entry into eukaryotic cell mitochondria, whereas NifD polypeptides having the sequence RRNX (SEQ ID NO:154) where X was any amino acid other than tyrosine (Y) would be less subject to secondary cleavage within that sequence. Those NifD sequences were therefore not preferred on the basis of their propensity to secondary cleavage. On the contrary, NifD sequences comprising any amino acid other than tyrosine (Y) at the position corresponding to Y100 of the K. oxytoca NifD (SEQ ID NO:18) were preferred, on the basis that they were likely to be resistant to cleavage upon introduction into eukaryotic cell mitochondria. Such sequences can readily be tested to confirm their resistance to cleavage within this region when expressed within plant cells as a MTP-NifD fusion polypeptide.

On further examination of the 1474 sequences, there were 155 sequences (10.51%) having the sequence RRNQ (SEQ ID NO:103) and 95 sequences (6.45%) having the sequence RRNK (SEQ ID NO:104), both of which were considered to not be subject to secondary cleavage and therefore more preferred than the sequences not having glutamine or lysine in the fourth position. These NifD polypeptides were considered more preferred than NifD polypeptides having the sequence RRNF (SEQ ID NO:220). It was then noticed that 141 of the 155 of the NifD sequences comprising the sequence RRNQ (SEQ ID NO:103) had a threonine (T) immediately after, i.e. comprised the sequence RRNQT (SEQ ID NO:105). On the basis that the polypeptide encoded by Var 24 (SN117) comprising the sequence RRNQT (SEQ ID NO:105) was not cleaved at that position and that the sequence was relatively frequent in naturally occurring NifD polypeptides, it was concluded that NifD polypeptides comprising the sequence RRNQT (SEQ ID NO:105) were highly preferred for use in eukaryotic cell mitochondria.

TABLE 11
Representative NifD sequences from a wide range of diazotrophic organisms.

| UniProt | | | |
|---|---|---|---|
| accession | Organism | Length | Phylum |
| A0A1S6IX91 | Desulfotomaculum ferrireducens | 539 | Firmicutes |
| Q1K0I7 | Desulfuromonas acetoxidans (strain DSM 684/ | 480 | Proteobacteria |
| | 11070) | | |
| C6E9A1 | Geobacter sp. (strain M21) | 480 | Proteobacteria |
| B0TAP4 | Heliobacterium modesticaldum (strain ATCC | 486 | Firmicutes |
| | 51547/Ice1) | | |
| Q0RAV5 | Frankia alni (strain ACN14a) | 486 | Actinobacteria |
| A0A089HSS3 | Paenibacillus durus (Paenibacillus azotofixans) | 485 | Firmicutes |
| A0A166T5M1 | Leptolyngbya valderiana BDU 20041 | 481 | Cyanobacteria |
| P00464 | Nostoc sp. (strain PCC 7120/SAG 25.82/UTEX | 497 | Cyanobacteria |
| | 2576) | | |
| A0A060DN91 | Azospirillum brasilense | 479 | Proteobacteria |
| P19066 | Sinorhizobium fredii (strain NBRC 101917/ | 504 | Proteobacteria |
| | NGR234) | | |
| C1DGZ7 | Azotobacter vinelandii (strain DJ/ATCC BAA- | 492 | Proteobacteria |
| | 1303) | | |
| A0A378D475 | Klebsiella oxytoca | 483 | Proteobacteria |
| A0A2J817Q6 | Vibrio diazotrophicus | 488 | Proteobacteria |
| A0A1D7TJ51 | Sulfurospirillum halorespirans DSM 13726 | 484 | Proteobacteria |
| E4TZQ3 | Sulfuricurvum kujiense (strain ATCC BAA-921/ | 489 | Proteobacteria |
| | DSM 16994/JCM 11577/YK-1) | | |
| B8DR77 | Desulfovibrio vulgaris (strain Miyazaki F/DSM | 544 | Proteobacteria |
| | 19637) | | |
| A0A0E3R6R4 | Methanosarcina barkeri 227 | 532 | Euryarchaeota |
| A0A0W1JIT9 | Desulfitobacterium hafniense | 493 | Firmicutes |
| | (Desulfitobacterium frappieri) | | |
| Q8KC89 | Chlorobaculum tepidum (strain ATCC 49652/ | 543 | Chlorobi |
| | DSM 12025/NBRC 103806/TLS) (Chlorobium | | |
| | tepidum) | | |
| A0A327XSU0 | Halanaerobium saccharolyticum | 539 | Firmicutes |
| A0A162KU04 | Clostridium ljungdahlii | 536 | Firmicutes |

TABLE 12
Frequency distribution of amino acid residues in the 1476 naturally occurring NifD sequences around the secondary cleavage site.

| | K. oxytoca amino acid | | | | | |
|---|---|---|---|---|---|---|
| | R97 | R98 | N99 | Y100 | Y101 | T102 |
| | Position relative to cleavage site | | | | | |
| | −3 | −2 | −1 | 1 | 2 | 3 |
| most common | R, 1474 (99.86%) | R, 1472 (99.73%) | N, 1444 (97.83%) | Y, 1056 (71.54%) | Y, 951 (64.43%) | V, 402 (27.24%) |
| 2nd most common | Gap, 2 (0.14%) | Gap, 2 (0.14%) | H, 17 (1.15%) | Q, 159 (10.77%) | A, 240 (16.26%) | I, 374 (25.34%) |
| 3rd most common | | C, 1 (0.07%) | F, 7 (0.47%) | L, 114 (7.72%) | T, 148 (10.03%) | T, 147 (9.97%) |
| 4th most common | | G, 1 (0.07%) | A, 3 (0.20%) | K, 108 (7.32%) | M, 44 (2.98%) | K, 135 (9.15%) |
| 5th most common | | | X, 2 (0.14%) * | F, 27 (1.83%) | F, 38 (2.57%) | R, 113 (7.66%) |
| 6th most common | | | Gap, 2 (0.14%) | M, 10 (0.68%) | G, 27 (1.83%) | N, 96 (6.50%) |
| 7th most common | | | S, 1 (0.07%) | E, 1 (0.07%) | S, 14 (0.95%) | D, 68 (4.61%) |
| 8th most common | | | | Gap, 1 (0.07%) | V, 12 (0.81%) | S 40 (2.71%) |
| 9th most common | | | | | N, 1 (0.07%) | E, 27 (1.83%) |
| 10th most common | | | | | Gap, 1 (0.07%) | Q, 23 (1.56%) |
| 11th most common | | | | | | L, 20 (1.36%) |
| 12th most common | | | | | | A, 12 (0.81%) |
| 13th most common | | | | | | H, 9 (0.81%) |
| 14th most common | | | | | | M, 8 (0.61%) |
| 15th most common | | | | | | Y, 2 (0.14%) |

* “X” meaning an unknown amino acid is present in the sequences Methylocella palustris (Q6KCQ3) and Methylosinus trichosporium (Q6KCQ2).

### Example 9. Functional Testing of NifD Variants Around the Secondary Cleavage Site

The MTP-FAγ51::NifD variants that did not show cleavage at the presumed site between residues 99 and 100 were tested for NifD function in the MIT2.1 system in E. coli, as follows. In order to introduce the mutations encoding the amino acid substitutions into the NifD gene in pMIT2.1 and to allow for easier cloning, restriction sites for the enzymes AgeI and SalI were introduced into the NifD coding region spanning the sites for the amino acid changes. This was done by PCR-mediated mutagenesis using oligonucleotide primer combinations 5′-CTAATGCTACCGGTGAACGTAACCTGGCACTGATTCAAGAAGTACTGGAAG TGTTC-3′ (SEQ ID NO:108) and 5′-GTTACGTTCACCGGTAGCATTAGTCATCATCCGG CTCCTCCGCTAGATAAAAATGTG-3′ (SEQ ID NO:109) for AgeI insertion, and 5′-GTTTCTGGCGTCGACTCTTTCGGCACGCTGAACTTCACCTCTGACTTCCAGG AAC-3′ (SEQ ID NO:110) and 5′-CGAAAGAGTCGACGCCAGAAACGCCCGTGTAGTAGTTA CGACGTCCCGCGCG-3′ (SEQ ID NO:111) for SalI insertion into the NifD gene of the pTopoH-J construct (Example 4). This AgeI to SalI fragment was codon optimised for N. benthamiana expression. The resulting vector was digested with SbfI, and ligated with SbfI digested B-ori, creating the positive control vector designated pS0043, encoding wild-type NifD as well as the other Nif polypeptides.

The AgeI-SalI region of NifD containing each of the amino acid substitutions was amplified by PCR in order to add AgeI and SalI restriction sites at the same positions as in p50043. This used primers 5′-GACCAATGCTACCGGTGAGAGGAACC-3′ (SEQ ID NO:112) and 5′-GTTAAGAGTCCCGAAAGAGTCGACACCAG-3′ (SEQ ID NO:113) and DNA from the constructs SN114, SN118, SN119, SN120, SN123, SN124 and SN125 as template, each encoding a different variant NifD sequence. The amplified AgeI-SalI NifD variant fragments were then ligated into pS0043 digested with AgeI and SalI, resulting in the series of constructs designated pS0044-050. These constructs thereby contained the AgeI-SalI region which was codon optimised for expression in plant cells whereas the rest of the NifD gene was codon optimised for expression in E. coli. Two other NifD vectors were also constructed in a similar manner using SN100 (NifD-Var13 having amino acid residues 99 and 103 substituted with five alanine residues) and EC38014, having a N. benthamiana codon optimised NifD gene, as DNA templates, resulting in p50052 and pS0053, respectively.

The bacterial expression constructs having variant NifD genes were introduced into E. coli strain JM109 along with the expression induction vector pN249 and tested for nitrogenase function using the acetylene reduction assay (ARA). The bacteria co-transformed with pSO053 (positive control, wild-type NifD) and the NifD variants encoded by pSO052 (alanine substitutions of residues 99 to 103), pSO044 (Y100Q), pSO045 (NYY99-101HKG), pSO046 (Y100K), and pSO047 (YY100-101KA) each produced ethylene to some extent. The amount of ethylene produced by pSO044, pSO045, pSO046 and pSO047 was 147%, 33%, 94%, and 67%, respectively, compared to the positive control. pSO052 also produced ethylene at 14% of the positive control. However, the E. coli cells containing pSO048, pSO049 and pSO050, all including the substitution R98K, produced only traces of ethylene at rates greater than the negative control, indicating that those NifD mutants were almost inactive for nitrogenase. In analogous fashion, a construct having the YY100-101QT double substitution yielded 107% ARA activity relative to the wild-type NifD control (Table 10). Therefore, both the Y100Q and YY100-101QT substitutions yielded increased NifD activity relative to the wild-type NifD sequence. It was concluded that arginine at position 98 was required for NifD function, consistent with its complete conservation in naturally occurring NifD sequences where activity could be assumed.

It was concluded more generally that NifD variants had been identified which retained substantial NifD function, indeed full or even increased NifD function for some variants, which were not subject to the secondary cleavage observed with the wild-type K. oxytoca NifD sequence. It was also concluded that the resistance of the NifD polypeptides to the secondary cleavage in plant mitochondria was combinable with increased nitrogenase activity, the latter being demonstrated for the modified NifD sequence in a bacterial system. It was also concluded that other NifD variants could be identified which were not subject to the secondary cleavage but which had lost NifD function.

### Example 10. Other NifD Polypeptides

### Modelling of the NifD-NifK Structure Around the Secondary Cleavage Site in NifD.

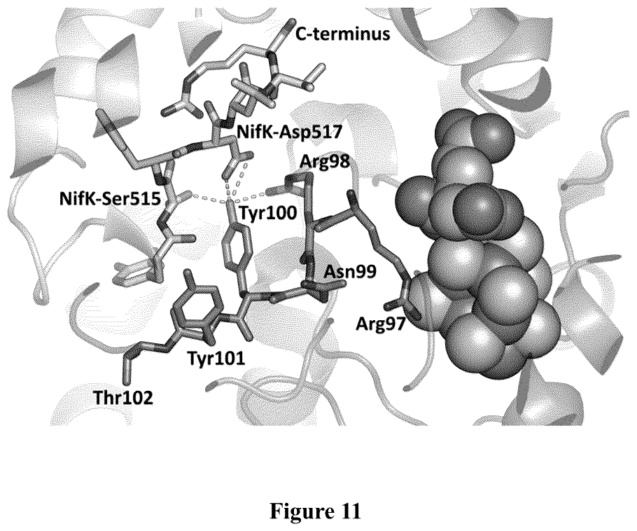

The protein structure of NifD polypeptide from K. oxytoca provided in PDB: 1QGU was visualised using PyMOL software, in particular focusing on the structure around the secondary cleavage site when NifD was bound with NifK polypeptide from K. oxytoca. The secondary cleavage site was observed to be located at the interface of NifD and NifK polypeptides, internally in the complex, and in close proximity to the essential cofactor FeMoco (FIG. 11). In the resting state, the Arg97 residue of NifD was coordinated to the bridging sulfido ligand (S5) located between Fe₃ and Fe₇ of FeMoco, explaining why Arg97 was entirely conserved in functional NifD polypeptides. It was thought to play an important role in stabilising the negative charges of the more reduced edge of the cluster (Fe1-Fe₃-Fe₇) (Spatzal et al., 2016). The hydroxyl group of Tyr100 in NifD formed hydrogen bonds to the amino group in Arg98 in NifD, the hydroxyl group in Ser515 in NifK and the carboxy group of Asp517 in NifK from K. oxytoca, also showing its importance for NifD function.

Homology models for the NifD variants having the Y100Q and Y100K substitutions were prepared using the SWISS-MODEL server (Waterhouse et al., 2018). The sequence of NifD Y100Q or NifD Y100K was used as target sequence. The sequence of NifK from K. oxytoca was again added to the model as a hetero target. For the Y100Q variant, the model predicted that the amino group of Gln100 of the NifD polypeptide formed a hydrogen bond to the carboxy group of Asp517 in NifK and the backbone carbonyl oxygen atom of Tyr514 in NifK. For the Y100K variant the model predicted that the amino group of Lys100 also forms hydrogen bonds to Asp517 and Tyr514 in NifK. The interaction of Tyr100 with Ser515 in NifK was replaced by an interaction of Gln100 or Lys100 with the backbone oxygen atom of Tyr514 in NifK. These observations were consistent with the retention of NifD activity for the Y100Q and Y100K substitutions.

The observations that (i) the sequence around the secondary cleavage site was internal in the NifD polypeptide when folded in its active conformation and (ii) that the NifD-linker-NifK polypeptide including the wild-type K. oxytoca NifD sequence was cleaved suggested that the secondary cleavage was occurring while the polypeptide was unfolded or being unfolded in the mitochondria.

The phylogenetic analysis described above (Example 8) showing that the amino acid residues leucine, phenylalanine, methionine and glutamic acid were also represented in naturally occurring NifD polypeptides at the position corresponding to Y100 in K. oxytoca NifD i.e. the +1 position relative to the secondary cleavage site and those NifD polypeptides were presumed to be functional. In those polypeptides, the amino acid Leu at the position corresponding to amino acid 100 was followed at position 101 by alanine (53 sequences), methionine (41 sequences), valine (10 sequences), threonine (4 sequences), phenylalanine (4 sequences) or tyrosine (2 sequences). When the amino acid corresponding to position 100 was Phe, the following amino acid was usually alanine (23 sequences) and in a few cases serine (2 sequences) or tyrosine (2 sequences). Met at position 100 was followed by alanine (3 sequences), methionine (3 sequences), glycine (2 sequences), valine (1 sequence) or threonine. Glu at position 100 was followed by threonine (1 sequence). However, the presence of Phe, Leu or Met at position 100 would not provide the hydrogen bonds that Y100 had with amino acids Ser515 and Asp517 in NifK from K. oxytoca.

To test their function, genetic constructs comprising the Y100L, Y100F and Y100M substitutions were made to the NifD sequence of K. oxytoca in an analogous fashion to the substitutions described above. These constructs encoding NifD variants were tested for the secondary cleavage phenotype after introduction into N. benthamiana leaf cells and for NifD function in E. coli using the pMIT2.1 system, in analogous fashion to that described in Example 8. All three of these NifD polypeptides having substitutions Y100L, Y100F and Y100M were still subject to secondary cleavage, showing that the amino acid sequences at the site were still recognised by MPP in the plant mitochondria. The other 14 possible substitutions at position 100 are readily tested in analogous fashion.

### Prediction and Testing of Naturally Occurring NifD Sequences for Cleavage

On the basis of the mutational and phylogenetic analyses, predictions were made about different naturally occurring NifD sequences, whether they would be cleaved or not cleaved, or less cleaved, in the region corresponding to amino acids 97-102 of the K. oxytoca NifD (SEQ ID NO:18). To test these predictions, one sequence was selected from each of the three clusters of NifD sequences that contained the highest number of members having the RRNYY sequence (SEQ ID NO:102), predicted to be cleaved. These selected NifD sequences were from Azotobacter vinelandii, Azospirillum brasilense, and Sinorhizobium fredii. These sequences are provided as SEQ ID NOs:148-150. Three other NifD amino acid sequences were identified which did not have the RRNY sequence (SEQ ID NO:101), instead having RRNQ (SEQ ID NO:103), RRNK (SEQ ID NO:104) or RRFK (SEQ ID NO:106) at the corresponding sequence (Table 13). These selected NifD sequences were from Clorobium tepidum, Desulfotomaculum ferrireducens and Desulfovibrio vulgaris, contained either a glutamine or lysine residue rather than tyrosine in the equivalent of position 100. These sequences are provided as SEQ ID NOs:151-153. It was predicted these three polypeptides would be less subject to cleavage within those sequences.

These selected sequences were aligned with the K. oxytoca sequence using Emboss Needle Pairwise Alignment Tool to determine the extent of identity, also shown in Table 13. It was noted that SEQ ID Nos:151-153 that did not have the RRNY sequence (SEQ ID NO:101) were less than 40% identical to SEQ ID NO:18.

To test the prediction in each case about the extent of secondary cleavage, genetic constructs (SN221-226) were made encoding MTP-FAγ51::NifD::HA fusion polypeptides where the NifD sequence was the same as the naturally occurring sequence, for introduction into N. benthamiana leaves. Protein extracts were prepared 5 days after infiltration and subjected to SDS-PAGE and Western blotting. It was observed that the fusion polypeptides comprising the RRNYY sequence (SEQ ID NO:102) from A. vinelandii and S. fredii NifD yielded intense secondary cleavage bands in the Western blots, with much more than 50% of the polypeptides cleaved at the secondary site, whereas the NifD polypeptide from D. vulgaris not having RRNYY (SEQ ID NO:102) showed little secondary cleavage. Polypeptides from C. tepidum and D. ferrireducens showed some secondary cleavage but less than for A. vinelandii and S. fredii NifD

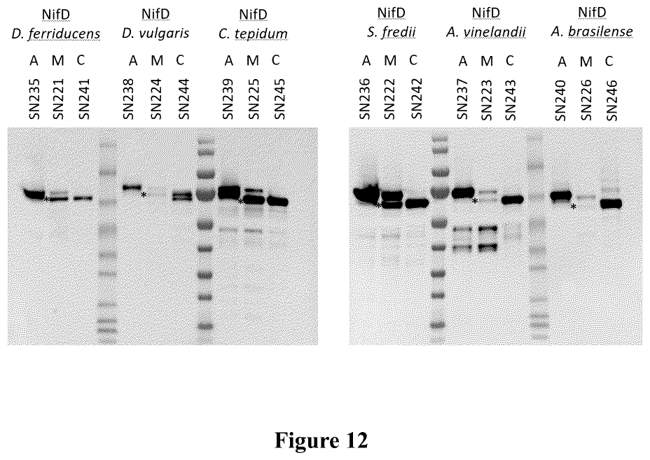

The experiment was repeated, including genetic constructs as controls which encoded fusion polypeptides that would not be cleaved with MPP due to alanine substitutions in the MTP-FAγ51 sequence (lanes marked A in FIG. 12) or that lacked the MTP sequence but had a 6×His motif instead (lanes marked C), equal in size to the MPP-processed forms. The Western blot analysis (FIG. 12) again showed that the ratio of full-length NifD and the secondary cleavage product varied for all six of the NifD fusion polypeptides. Buren et al. (2017b) observed, in yeast mitochondria, a degradation product of NifD from A. vinelandii of approx. 48 kDa. The size of that polypeptide was in line with the predicted secondary cleavage by MPP at the RRNYY (SEQ ID NO:102) site. In the repeat experiment, a degradation product of this size was observed for the A. vinelandii NifD polypeptide. A second degradation product was also observed at approximately 40 kDa molecular weight, suggesting the presence of a second cryptic cleavage site. The two degradation products were not observed for the genetic construct encoding the cytoplasmically localised fusion polypeptide 6×His::NifD::HA, indicating that the second degradation product also arose from mitochondrial protease activity. A difference was also noted between the processing in plant mitochondria compared to the yeast mitochondria. Further studies are required to determine the origin of the second degradation product of the A. vinelandii AvNifD fusion polypeptide in plant cells. The fusion polypeptide including the NifD sequence from A. brasilense was mainly present in the unprocessed form, indicating that the import efficiency of the AbNifD fusion polypeptide into the mitochondria was low. For this reason, it was difficult to assess the amount of MPP cleavage for that one. Significantly, this time only a faint degradation product was observed for the fusion polypeptide including the SfNifD sequence from S. fredii.

The relative abundance of the MTP51::NifD::HA polypeptides varied extensively even though the same promotor was used in all of the genetic constructs. The abundance of the NifD fusion polypeptides from A. brasilense, A. vinelandii and D. vulgaris was low compared to the NifD fusion polypeptides from C. tepidum and S. fredii.

The experiment was extended by fractionating the protein extracts into soluble and insoluble fractions as described in Example 1. It was observed that the NifD fusion polypeptides from C. tepidum, D. ferrireducens and S. fredii were soluble to some extent, up to about 50% for D. ferrireducens NifD.

It was concluded that naturally occurring NifD sequences having the RRNYY sequence (SEQ ID NO:102) were less preferred due to their tendency for secondary cleavage within that sequence in plant mitochondria, but that exceptions such as the S. fredii NifD can be found.

TABLE 13
Wild-type NifD polypeptide sequences at the sequence corresponding to K.
oxytoca amino acids 97-102. The % amino acid sequence identity to SEQ ID NO: 18 is
shown. The first three sequences comprising RRNYY (SEQ ID NO: 102) were predicted
to be cleaved whereas the last three sequences were predicted to not be cleaved within
this region.

| | Genbank | | % | |
|---|---|---|---|---|
| Organism | Accession No. | Length | identity | Sequence at site |
| Azospirillum | WP_014239785 | 479 | 69.7 | RRNYY |
| brasilense | (SEQ ID | | | (SEQ ID NO: 102) |
| | NO: 148) | | | |
| Azotobacter | WP_012698832 | 492 | 71.8 | RRNYY |
| vinelandii | (SEQ ID | | | (SEQ ID NO: 102) |
| | NO: 149) | | | |
| Sinorhizobium | WP_010875129 | 504 | 63.8 | RRNYY |
| fredii | (SEQ ID | | | (SEQ ID NO: 102) |
| | NO: 150) | | | |
| Chlorobium | WP_010933201 | 543 | 39.9 | RRNQT |
| tepidum | (SEQ ID | | | (SEQ ID NO: 105) |
| | NO: 151) | | | |
| Desulfovibrio | WP_015946243 | 544 | 35.3 | RRNQT |
| vulgaris | (SEQ ID | | | (SEQ ID NO: 105) |
| | NO: 152) | | | |
| Desulfotomaculum | WP_077714465 | 539 | 38.4 | RRNKA |
| ferriducens | (SEQ ID | | | (SEQ ID NO: 107) |
| | NO: 153) | | | |

### Example 11. NifD Variants in the Context of NifD-NifK Fusion Polypeptides

The effect of the Y100Q substitution on NifD processing and function in the context of a NifD-linker-NifK fusion polypeptide was also tested. To do this, the pMIT2.1 vector was first modified to translationally fuse the otherwise wild-type NifD and NifK coding regions, as follows. The operon structure between the NifD and NifK genes in pMIT2.1, encoding separate NifD and NifK polypeptides, was replaced with a nucleotide sequence to provide for a translational fusion of the NifD and NifK polypeptides, joined by a 30 amino acid linker (ATPPPGSTTTAYPYDVPDYATPPPGSTTTA, SEQ ID NO:116) which included a HA epitope tag (YPYDVPDYA, SEQ ID NO:115). The DNA fragment encoding this NifD::linker(HA)::NifK polypeptide was from the NifD::FLAGlinker::NifKs gene (Allen et al., 2017) except that the nucleotide sequence coding for the amino acids of the FLAG epitope was replaced with a sequence encoding the HA epitope, forming a vector herein designated as pTopoH-J-DHAK. After that, the second, unmodified half of pMIT2.1 (NifB-ori) digested with SbfI was ligated with pTopoH-J-DHAK after digestion with SbfI, resulting in p50018. This construct therefore encoded the translational fusion of NifD::finker(HA)::NifK with all of the other Nif genes as in pMIT2.1, the NifD amino acid sequence being unmodified with respect to the wild-type K. oxytoca sequence.

Introduction of the Y100Q mutation in the NifD coding region in pTopoH-J and pTopoH-J-DHAK was achieved by mutagenesis using primers 5′-GTCGTAACCAATACACGGGCGTTTCTGGCGTCGACTCTTTCGGCACG-3′ (SEQ ID NO:117) and 5′-GCCCGTGTATTGGTTACGACGTCCCGCGCGAGAG TACTGGC-3′ (SEQ ID NO:118) to make the nucleotide substitutions T298C and C300A, changing the tyrosine (Y) codon TAC to the glutamine (Q) codon CAA. The resulting pTopoH-J vectors encoding unfused or fused NifD(Y100Q) were digested with SbfI and ligated with NifB-ori also digested with SbfI, creating pS0054 which was the reformed pMIT2.1 encoding NifD(Y100Q) and p50055 which was the reformed pMIT2.1 encoding the NifD(Y100Q)::linker(HA)::NifK translational fusion polypeptide.

These genetic constructs were tested with the acetylene reduction assay in E. coli. pSO054 (encoding unfused NifD(Y100Q)) and pSO055 (encoding the fused NifD(Y100Q)::linker(HA)::NifK) produced ethylene at between 80% and 90% compared to their respective positive controls pS0005 and pSO018. This demonstrated that the Y100Q mutation did not compromise NifD activity in the context of the NifD::linker::NifK fusion polypeptide, with the activity being reduced only slightly.

### Example 12. Solubility of Wild-Type NifD and Sequence Variants in Plant Mitochondria

Example 3 describes experiments showing that many of the Nif polypeptides expressed in the form of an MTP::Nif fusion polypeptide for mitochondrial localisation were essentially insoluble or only sparingly soluble when expressed as a single polypeptide. The data also demonstrated that the process of targeting Nif fusion polypeptides to the mitochondria or the mitochondrial environment itself, or both, negatively influenced Nif polypeptide solubility for at least NifD, NifH and NifK, relative to cytoplasmic localisation. As solubility of nitrogenase protein components in the mitochondrial matrix is considered to be a prerequisite for functional reconstitution of nitrogenase in the mitochondria of eukaryotic cells, the inventors sought to determine the reasons for these observations on Nif polypeptide solubility. In particular, considering the importance of NifD, NifK and NifH, several approaches were tested to increase the solubility of these crucial polypeptides as described below. Insolubility in the mitochondria could be a consequence of incorrect protein folding, improper glycosylation or other post-translational modification, formation of aggregates or association with cell membranes, or a combination of these or other reasons.

### Solubility of NifD Fusion Polypeptides—Effect of Promoter, MTP and NifD Sequences

Initially, a set of plant expressed MTP::NifD polypeptides were tested for solubility, including whether N- and C-terminal modifications might influence solubility of the MPP-processed and unprocessed forms. For this purpose, a range of MTP::NifD constructs including some described above were infiltrated into N. benthamiana leaves via A. tumefaciens (Table 14). These constructs varied in the promoter for expression (e35S or SCSV S₄ promoters) or in the encoded MTP or NifD polypeptide sequences (cleaved or non-cleaved at the secondary site). All of them contained a HA epitope tag fused to the C-terminus of the NifD polypeptide except for the polypeptide encoded by SN75 which had a HA epitope sequence fused to each of the N-terminus and C-terminus of NifD, thus flanking the NifD polypeptide. As a positive control for a soluble NifD polypeptide, the genetic construct SN33 (Example 3) encoding the non-mitochondrial targeted version of NifD was also infiltrated. In each case, the construct SN46 encoding the MTP-Su9::NifK was co-infiltrated with the NifD construct to enhance NifD accumulation. For each infiltration, proteins were extracted from each leaf spot and fractionated into soluble and insoluble fractions as described in Example 1, as well as retaining some unfractionated samples (“total protein”). Samples were analysed by loading them in adjacent lanes on SDS-PAGE gels and Western blotting using anti-HA to detect the MPP-processed and unprocessed MTP::NifD::HA and MTP::HA::NifD::HA polypeptides.

From the Western blots, the non-mitochondrially targeted HA::NifD polypeptide produced from SN33 was almost completely soluble (solubility score of 4, Table 14). In contrast the MTP-FAγ::NifD::HA polypeptide encoded by SN10 and its MPP-processed derivative were either not detected or barely detected in the soluble fraction, so were essentially insoluble. Modifying the promoter by substituting the SCSV S₄ promoter (SN06) for the e35S promoter appeared to slightly increase the amount of soluble NifD::HA polypeptide. Changing the MTP by replacing the FAγ51 sequence with the CPN60 or Su9 MTP sequences (5N₀₄, SN14) did not noticeably increase NifD solubility. A slight increase in solubility was observed when the Y100Q amino acid substitution was incorporated into the NifD amino acid sequence (SN114). None of these modifications had a major effect. However, the standout change in both NifD expression level and solubility occurred with SN75. At least 50% of the fusion polypeptide encoded by SN75, containing a HA epitope tag between the MTP-FAγ51 and the NifD sequence as well as a second, C-terminal HA epitope tag, was in the soluble fraction. Curiously, a different N-terminal epitope located between the MTP and NifD produced a different result—the construct encoding the MTP-CoxIV::TwinStrep::NifD:HA polypeptide (SN19) yielded mostly insoluble NifD polypeptide.

In view of the result with SN75, a similar construct was made (SN140) for NifK expression, having a Gly-Gly linked HA epitope tag placed between the MTP-FAγ51 (SEQ ID NO:36) and the NifK sequences. After infiltration of SN140 into N. benthamiana leaf cells, soluble, insoluble and total protein fractions were prepared. However in contrast to SN75, SDS-PAGE and Western blotting showed that the NifK fusion polypeptide remained insoluble. This result demonstrated that the insertion of the HA linker-GG into the fusion polypeptide had different effects on protein solubility depending on its Nif polypeptide, in this case NifD vs NifK.

Overall, these results confirmed that the process of targeting NifD polypeptides to the mitochondria or the mitochondrial environment itself negatively affected NifD solubility. They also showed that a N-terminal modification could overcome this problem at least in part.

TABLE 14
Solubility of MTP::NifD fusion polypeptides after expression
in plant cells and processing of the MTP by MPP, as determined
by SDS-PAGE and Western blot analysis of soluble and insoluble
fractions. Solubility scores were given for each fusion polypeptide:
0, no soluble full length NifD polypeptide detected; 1, soluble
polypeptide only just detected; 2, soluble polypeptide detected
but <50% of total polypeptide; 3, soluble polypeptide detected
at >50% of total polypeptide; 4, at least 90% of total polypeptide
detected as soluble polypeptide.

| Construct | | | Secondary | Epitope | Solubility |
|---|---|---|---|---|---|
| ID | Promoter | MTP | cleavage | tag(s) | score |
| SN33 | e35S | None | Yes | 5′HA | 4 |
| SN10 | e35S | FAγ51 | Yes | 3′HA | 1 |
| SN06 | S4V2 | FAγ51 | Yes | 3′HA | 2 |
| SN04 | e35S | CPN60 | Yes | 3′HA | 0 |
| SN14 | e35S | Su9 | Yes | 3′HA | 0 |
| SN19 | e35S | CoxIV | Yes | 5′TwinStrep | 0 |
| | | | | 3′HA | |
| SN53 | e35S | FAγ51 | Yes | 3′HA | 0 |
| SN58 | e35S | FAγ51 | Yes | 3′HA | 0 |
| SN75 | e35S | FAγ51 | Yes | 5′HA + | 3 |
| | | | | 3′HA | |
| SN114 | e35S | FAγ51 | No | 3′HA | 2 |
| | | | (Y100Q) | | |
| SN100 | e35S | FAγ51 | No | 3′HA | 0 |
| SN96 | e35S | FAγ51 | Yes | 3′HA | 2 |
| SN97 | e35S | FAγ51 | Yes | 3′HA | 2 |
| SN99 | e35S | FAγ51 | Yes | 3′HA | 1 |
| | | | (reduced) | | |

### Solubility of NifD::Linker::NifK Fusion Polypeptides

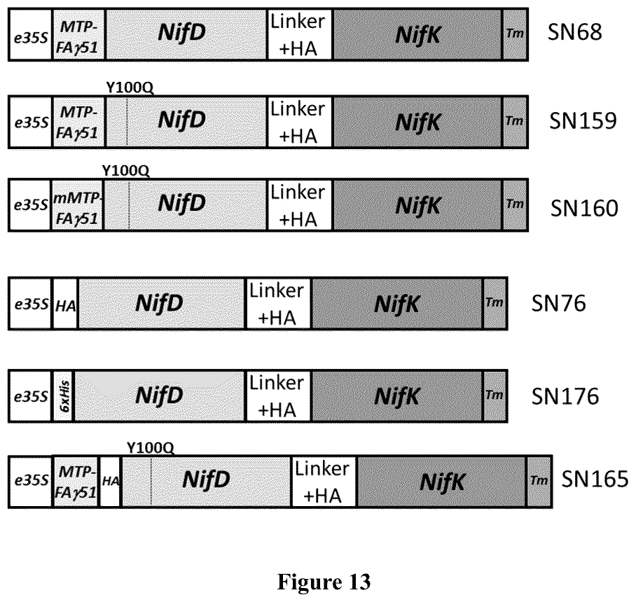

Given these results, the effect on NifD solubility of another C-terminal extension was tested, namely the addition of a NifK sequence to provide a MTP::NifD::linker(HA)::NifK translational fusion (Allen et al., 2017). For this, the genetic construct SN68 was made that, like SN10, contained the strong e35S promoter for expression and a TMV-omega 5′-UTR region for efficient translation (Gallie et al., 1987). SN68 encoded a fusion polypeptide that had the MTP-FAγ51 with a Gly-Gly linker fused to the NifD N-terminus, then a 30 amino acid linker comprising the HA epitope tag sequence as previously used in pRA20, followed by the NifK sequence. This is shown schematically in FIG. 13. The NifD amino acid sequence was according to SEQ ID NO:18. The protein coding region was codon-optimised for expression in N. benthamiana.

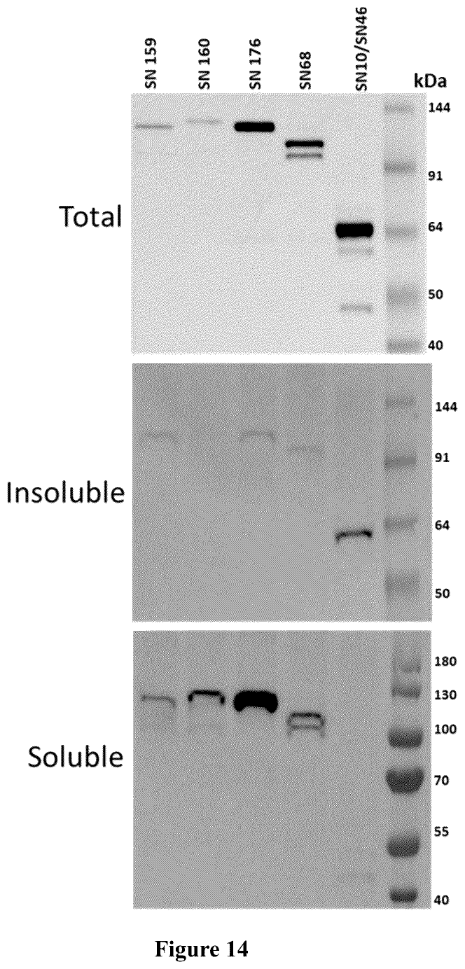

This polypeptide was tested for solubility by infiltration of SN68 into N. benthamiana and isolation of soluble, insoluble and total protein fractions. SDS-PAGE with Western blot analysis was performed on the protein fractions. Two bands appeared on the blots (FIG. 14) which were slightly smaller than expected and probably represented cleavage at the secondary cleavage site within the NifD sequence. However, despite this, it was observed that most of the fusion polypeptides comprising the HA epitope and the NifK sequence were in the soluble fraction and only a small amount in the insoluble fraction (FIG. 14). This was the first time that the inventors had observed a mostly soluble NifK polypeptide.

Since the SN68-encoded polypeptide included the amino acid sequence that was susceptible to the secondary cleavage between amino acids 97-102 of NifD, a second, corresponding construct was made that contained the Y100Q amino acid substitution that had been shown to protect NifD from the secondary cleavage in mitochondria (Example 6). This genetic construct was designated SN159. In order to distinguish processed and unprocessed fusion polypeptides on the SDS-PAGE gels and thereby establish whether the fusion polypeptide encoded by SN159 was cleaved by MPP within the MTP sequence, a third construct was made identical to SN159 except that the MTP-FAγ51 sequence was modified with alanine substitutions that would render it resistant to mitochondrial processing by MPP. The same alanine substitutions within MTP were made as in the SN66 polypeptide. The third construct, SN160, was thus designed to produce a mMTP::NifD::linker(HA)::NifK fusion polypeptide which would not be processed by MPP and therefore yield a larger size product than a processed product from SN159. Further, a fourth, control construct was made, designated SN176, encoding a fusion polypeptide that lacked an MTP sequence and therefore was not targeted to the mitochondria but rather would be cytoplasmically located. For this construct, the MTP-FAγ51 sequence of SN159 was replaced with a 6×His tag linked by two glycine's to the NifD start codon. The 6×His+Gly-Gly sequence was very similar in size to the scar sequence predicted to be produced from MTP-FAγ51 after MPP-processing. It was predicted that if SN159 was processed correctly, the protein products from SN176 and SN159 would be of virtually identical length (1040 residues/116,251 Da for SN176, 1042 residues/116,317 Da for processed SN159).

These constructs SN68, SN159, SN160 and SN176 were infiltrated separately into N. benthamiana leaves and, after 5 days, three protein fractions were prepared from each infiltrated leaf region: total protein, soluble protein and insoluble protein, as described in Examples 1 and 3. The fractions were analysed by SDS-PAGE and Western blotting with HA-antibody. The SDS-PAGE gels were run for longer than normal in view of the large sizes of the polypeptides, to provide for better resolution.

Both SN159 and SN160 yielded a distinct polypeptide band having a molecular weight of approximately 120 kDa, with the main polypeptide from SN160 noticeably larger than that from SN159. The polypeptide from SN159 appeared to be the same size as that produced from SN176 which lacked the MTP sequence. From this, it was concluded that the polypeptide produced from SN159 was efficiently processed by MPP. In contrast, the polypeptides produced from SN68 were smaller and therefore were presumed to include a product from secondary cleavage within the NifD sequence. It was predicted that the polypeptide produced from SN68, not having the Y100Q substitution, would be subject to secondary cleavage and therefore produce a product of 933 residues/104,403 Da. A polypeptide band of that size was observed.

Most gratifying, and surprising to the inventors, was the result from the solubility analysis for the polypeptides produced from these constructs. More of the polypeptide produced from SN159 was observed in the soluble fraction than in the insoluble fraction. This was the first time the inventors had seen this for a mitochondrially targeted NifD polypeptide. Moreover, the processed MTP::NifD::linker(HA)::NifK polypeptide had been shown to function for NifD and NifK in the bacterial assay system (Example 11). Therefore, the inventors concluded that they had successfully modified the Nif polypeptides to produce a soluble, functional polypeptide having both NifD and NifK functions which was resistant to the secondary cleavage of the NifD sequence within the RRNYY sequence (SEQ ID NO:102).

Besides solubility, there were some important processing differences between the individually expressed NifD and the NifD::linker::NifK polypeptides. Firstly, unlike the NifD polypeptide from SN10 and its substitution variants (Example 6), the MTP::NifD::linker::NifK polypeptide that contained wild-type NifD sequence (SN68) was fully processed by MPP. Even though the ˜48 kDa secondary cleavage product predominated from SN10 and some other NifD variant constructs, full length NifD polypeptide that had not been cleaved at the RRNYY (SEQ ID NO:102) site was always detected. Secondly, despite the use of the same MTP-FAγ51 for SN159, SN10 and other NifD variants, processing by MPP appeared complete for SN159, whereas both processed and unprocessed MTP-FAγ51::NifD were always observed when NifD was expressed on its own. Therefore, fortuitously, the fusion polypeptide from SN159 not only had resistance to the secondary cleavage in mitochondria and was predominantly soluble, it also appeared to be completely processed at the canonical site within the MTP sequence.

### Isolation of the NifD-Linker-NifK Fusion Polypeptide

The NifD::linker(HA)::NifK fusion polypeptide encoded by SN159 was isolated from N. benthamiana leaf samples by the following immuno-selective method. Twelve leaf pieces each approximately 2 cm 2 in size that had been infiltrated with SN159 were ground in 10 mL solubility buffer. The solubility buffer contained: 100 mM Tris pH 8.0, 150 mM NaCl, 0.25 M mannitol, 5% (v/v) glycerol, 1% (w/v) PVP40, 0.1% (v/v) Tween 20, 2 mM TCEP, 0.2 mM PMSF and 10 μM leupeptin. The use of the low level of detergent (0.1% Tween20) was expected to result in extraction of only soluble proteins. The ground mixture was centrifuged at 5500×g for 15 min at 4° C. and the supernatant transferred to a clean tube. Anti-HA agarose beads (Sigma) were washed once with buffer containing 50 mM Tris pH 8.0 and 75 mM NaCl (TN buffer) and the beads then added to the supernatant to immuno-precipitate polypeptides having the HA epitope. The mixture was incubated for 1 h at 4° C. with slow rotation and the beads allowed to settle. A sample of the supernatant was retained as the “unbound proteins”. The beads were washed 5 times with 1 mL of TN buffer each time, centrifuging each time at 1000×g for 2 min at RT to settle the beads. Finally, 60 μL of Laemmli buffer was added to the beads and the mixture heated at 95° C. for 5 min to release the bound proteins and denature them. Samples were loaded onto duplicate SDS-PAGE gels.

One of the gels was blotted onto a membrane and treated as a Western blot. An intense polypeptide band of the size expected for a MPP-processed NifD::linker(HA)::NifK polypeptide was observed as well as two less intense bands for smaller polypeptides that were considered to be degradation products, probably caused by protease cleavage at cryptic sites within NifD. Since the NifD sequence within the polypeptide had the Y100Q amino acid substitution, it was unlikely that the further protease cleavage/degradation was occurring at that site in NifD but rather at one or more new sites. The Western blot also showed two intense bands which were thought to represent the mouse Ig 50 kDa and 25 kDa polypeptides present in the anti-HA agarose beads used in the immuno-precipitation.

The second SDS-PAGE gel was stained with Coomassie stain and used to excise gel slices in the regions corresponding to the NifD::linker(HA)::NifK band (Sample 1) and the smaller degradation products (Sample 2). Proteins in these gel slices were digested with trypsin and analysed by LC-MS as described in Example 1. The extracted tryptic peptides were dried and resuspended in 30 μL of 1% formic acid. Initially, 5 μL of the tryptic peptides from each of the digests were injected on the 6600 Triple TOF MS using an Eksigent microHPLC (55 min). Residual tryptic peptides were stored at ˜20° C.

Data were processed using ProteinPilot against the species-specific UniProt Knowledgebase (UniProtKB) databases appended with the custom and adventitious databases: Uniprot-Nbenth+Custom Nif database+Common Repository of Adventitious Protein. Several specific peptides from the target polypeptide were positively identified in Sample 1, with 2 peptides from within the NifD sequence identified at the >95% confidence level and one other peptide from NifD and two other peptides within the NifK sequence identified with 94.9, 93.3 and 55.3% confidence levels. Two scar peptides derived from cleavage by MPP within the MTP sequence, ISTQVVR (SEQ ID NO:119) and SISTQVVR (SEQ ID NO:120), were not detected in the discovery data but were detected using the more sensitive targeted MRM on the 6500 Q-trap with 6 transition ions/peptide, at retentions times of 2.83 min and 3.15 min, respectively. Assessment of the dominant transition ions (+2y6) indicated that the peptide SISTQVVR (SEQ ID NO:120) was slightly more abundant than ISTQVVR (SEQ ID NO:119) in Sample 1.

It was concluded that Sample 1 indeed contained the MPP-processed NifD::linker(HA)::NifK polypeptide and that the polypeptide had been extracted from the N. benthamiana cells in a soluble form.

Sample 2 had a lower protein content and therefore the analysis was more difficult. Nevertheless, a single tryptic peptide was identified from within the NifD sequence and evidence for a second peptide from within NifD. The two scar peptides ISTQVVR (SEQ ID NO:119) and SISTQVVR (SEQ ID NO:120) were not detected in the discovery data and the more sensitive MRM on the 650 Q-trap. These data were consistent with the polypeptides in Sample 2 having resulted from an additional cleavage within the NifD sequence.

### Enhancement of Solubility of NifK Polypeptide

The inventors tested whether the co-expression of NifD and NifK polypeptides from separate constructs would enhance the solubility of the NifK polypeptide relative to expression of NifK without NifD. The experiments described above with SN140 (MTP-HA::NifK) had shown that the polypeptide was essentially insoluble when expressed alone. Therefore, N. benthamiana leaves were infiltrated with a mixture of A. tumefaciens strains separately transformed with SN140 and either SN10, SN114 or SN117. SN10 encoded the wild-type NifD sequence whereas SN114 and SN117 contained amino acid substitutions in NifD to reduce the secondary cleavage. Protein extracts containing soluble and insoluble fractions as well as non-fractionated proteins were analysed by SDS-PAGE and Western blotting, as before.

The blots showed that there was a substantial increase in the solubility of the NifK fusion polypeptide expressed from SN140 when co-introduced with any of the NifD constructs. In the absence of NifD, NifK was barely detectable in the soluble fraction, whereas, in the presence of NifD, about equal amounts of NifK were found in the soluble and insoluble fractions. It was concluded that solubility of MTP::NifK polypeptide was increased by co-expression of NifD together in the same plant cell, even when expressed as separate polypeptides. This added to the observation described above that the MTP-NifD-linker-NifK fusion polypeptide provided for a more soluble form of NifK polypeptide. It was also concluded that both observations pointed to an association of the NifD and NifK polypeptides in the mitochondrial matrix—necessarily for the NifD-linker-NifK fusion polypeptide, but also when expressed as separate polypeptides.

### Example 13. Purification of Plant Mitochondria Using a Fusion Protein and Magnetic Beads

The inventors conceived of a way to rapidly purify plant mitochondria in order to better investigate the localisation and function of exogenous polypeptides in mitochondria such as Nif polypeptides which they desired to introduce into that subcellular organelle. Traditional methods for isolation of highly enriched plant mitochondria typically require freshly harvested leaf material to be processed with various buffers followed by a sequence of centrifugation steps to remove non-mitochondrial components (Millar et al., 2007). Those methods required substantial amounts of starting material (e.g. 20 to 40 grams of plant material) and the entire process takes many hours before the purified mitochondria were ready for use or analysis. More rapid isolation methods starting with smaller amounts of plant material have been developed (Millar et al., 2007). However those methods are best considered as mitochondrial enrichments as the products usually still contain other cellular components (Carrari et al., 2003).

In the N. benthamiana leaf assays described herein, 8-10 or more “infiltration zones” can be applied to a single leaf with each zone capable of expressing single or multiple (up to about 8) transgenes introduced via a mixture of A. tumefaciens transformants. Such leaf assays were ideal for rapid-throughput testing of gene combinations and were generally predictive of metabolic pathways eventually designed for expression in stably transformed plants. Generally each infiltration zone was only 2-3 cm in diameter, resulting in an overall fresh weight of 50 to 100 mg per infiltration zone. Small amounts of fresh material such as these were not suitable for traditional plant mitochondrial preparations where the numerous steps result in substantial loss of mitochondria. Therefore the inventors established a protocol for one-step purification of plant mitochondria in less than 10 min from small samples such as 50-100 mg.

The outer membrane of plant mitochondria has various protein import and export machineries. Metaxin is a plant-specific protein of about 40 kDa found on the outer membrane of plant mitochondria and is possibly involved in the recognition of proteins prior to import into the mitochondria (Lister et al., 2004). The protein appears to be specifically located to mitochondria. Structurally, metaxin has a single membrane spanning region located towards the C-terminus of the protein with the N-terminus of the protein likely to be located in the plant cytoplasm. A fusion of GFP to the N-terminus of metaxin resulted in plant mitochondria with a fluorescent signal located to the outer membrane (Lister et al., 2004). The inventors considered that the N-terminus of metaxin, if indeed located within the cytoplasm, was likely to be accessible to antibody binding and might allow for an affinity tag based purification method. It was further considered that placing the epitope at the N-terminus of a reporter polypeptide such as the GFP variant mTurquoise would help push the epitope into the cytoplasm. The TwinStrep-tag was selected as the tag to add to the N-terminus. The Twin-Strep-tag Strepavidin interaction provided a specific and tight, yet reversible, binding with applications reported in affinity-based protein purification (Schmidt et al., 2013; Schmidt and Skerra, 2007). The Twin-Strep-tag as a translational fusion provided tight yet reversible binding to the engineered binding substrate StrepTactinXT, although it can also bind to streptavidin.

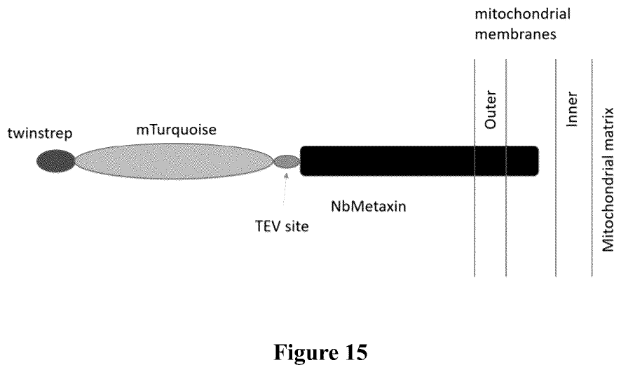

The inventors conceived of a fusion polypeptide with several components, shown schematically in FIG. 15. A genetic construct was designed and made encoding this fusion polypeptide. A combination of gene synthesis and the GoldenGate cloning methods was used to generate a genetic construct having a 35S promoter for expression in plant cells and encoding a TwinStrep-mTurquoise-TEV recognition sequence-metaxin fusion polypeptide (construct SN197, SEQ ID NO:121). The N-terminal Twin-Strep-tag epitope was included to enable antibody-mediated affinity purification, the mTurquoise component allowed for monitoring of the purification using confocal microscopy and also extended the N-terminus of metaxin further into the plant cytosol, and the TEV protease recognition sequence allowed in vitro TEV protease mediated cleavage of the polypeptide in order to release the plant mitochondria from the magnetic beads. Since wild-type metaxin becomes embedded in the outer membrane of the plant mitochondria, expression of the gene from SN197 in plant cells was thought to enable purification of this organelle, provided that the fusion protein would localise to the outer membrane of mitochondria. That was unknown until tested as described below.

A. tumefaciens cells containing SN197 were infiltrated into N. benthamiana leaves, together as part of a mixture of cells containing separate constructs for expression of the p19 silencing suppressor, MTP-FAγ::GFP (construct pRA01) and a cytoplasmically localised NifU::HA (SN211), each at an OD of 0.1 and therefore with a total OD of 0.4. Appropriate control mixtures having some but not all of the components, each with p19, were also infiltrated. After four days, infiltration zones were excised, providing samples of about 100 mg fresh weight in a 4 cm×2 cm leaf piece. The following steps were performed at 4° C. Leaf material was ground by hand in a mortar and pestle using 500 μL KPBS buffer. That buffer contained 5.07 g KCl and 0.68g KH₂PO₄ in 500 mL deionised water, adjusted to pH 7.25 using 1M KOH. The slurry was centrifuged at low speed, 1000 g for 5 min, to pellet cell wall debris but leaving most mitochondria in suspension. 300 μL of supernatant was applied to 50 μL of a slurry of magnetic beads coated with streptavidin (2.8 μm diameter, smooth coated beads, DynalBeads MyOne C1 product code 65002) in a 1.5 ml Eppendorf tube, after the beads had been washed once with KPBS buffer. At set times, the magnetic beads in the mixture were collected to the wall of the tube using a magnet and the remainder of the liquid was carefully removed. The magnetic beads were then washed twice with 1 mL of KPBS, each time collecting them as before with the magnet, and finally resuspended in 50 μL KPBS. As a control sample, the same bead purification protocol was applied to N. benthamiana leaf extracts expressing pRA01 (encoding MTP-FAγ77::GFP), SN211 (encoding cytoplasmic NifU::HA) and p19 but without SN197.

Several experiments were carried out to determine optimised conditions for mitochondrial purification using the magnetic beads. Firstly, various TwinStrep-binding bead products were compared. It was observed that MyOne C1 beads were superior to Dynalbeads MyWay T1, M-280 and M-270 and also to an IBT StreptaxtinXT-Agarose product. A time course of binding of the SN197 sample to C1-beads was conducted using 1, 5, 10, 30 or 60 min incubation, finding that maximal and saturated binding occurred after 5 min. No GFP signal was detected after the purification protocol for the samples where SN197 was omitted from the infiltration mixtures, indicating that there was no non-specific binding of mitochondria to the magnetic beads. Confocal microscopy showed that the fluorescent signals from mTurquoise (SN197) and GFP (MTP-FAγ77::GFP) was greatest in incubations with MyOne C1 beads. Different concentrations of C1 beads were incubated with the extracts. Recovery of TwinStrep::mTurquoise::TEV::Metaxin and MTP-FAγ::GFP was dependent upon bead concentration, saturating at 50 μl of MyOne C1 bead slurry, so that amount was subsequently used.

The steps in the purification process were analysed via confocal microscopy to assess the presence of GFP and mTurquoise polypeptides and autofluorescence from plant chloroplasts. GFP and mTurquoise were detected at excitation wavelengths of 488 nm and 434 nm, respectively. The samples coming from infiltrations with the combination SN197, pRA01 and p19, when ground with KPBS buffer and subjected to low speed centrifugation, were enriched for GFP-fluorescing mitochondria. Only a few intact subcellular organelles other than mitochondria, such as chloroplasts and nuclei, and few fragments of cellular debris were observed. After washing of the beads using 2 mL of KPBS buffer and magnetic pull-down, confocal microscopy of the resulting suspension showed that fluorescing mitochondria were physically attached to the beads. After this step in the purification, other organelles and cell debris were not observed.

The purification process was also analysed via Western blot assays. To do this, polypeptides bound to the magnetic beads were released and denatured by the addition of 100 μL of Laemmli buffer (Example 1) and heating the samples at 95° C. Samples of the plant extract after grinding but before purification, labelled the “input sample”, were also included in the Western blot analysis using antibody binding to GFP to detect MTP-FAγ::GFP and mTurquoise:metaxin polypeptides and anti-HA to detect the NifU::HA polypeptide. The Western blots showed that the TwinStrep::mTurquiose::TEV::Metaxin polypeptide was readily detected at a molecular weight of about 80 kDa, consistent with a single, intact translational fusion protein. A band was observed at about 30 kDa with the antibody to GFP for samples including pRA01, consistent with expected size of the mitochondrially targeted MTP-FAγ::GFP. Furthermore, a band at about 42 kDa was observed with the HA antibody from extracts having SN211, consistent with the expected size for the NifU::HA polypeptide. To check for non-mitochondrial proteins as potential contaminants, the abundance of the cytoplasmic protein α-tubulin was assessed with a corresponding antibody (Sigma Catalog No. T6074, clone B-5-1-2 monoclonal antibody). A specific band for this protein at about 52 kDa was only observed in lanes having the input sample; no α-tubulin signal was found in purified mitochondrial extracts showing that the purification was very good. It was concluded that use of the metaxin fusion polypeptide such as the one encoded by SN197 enabled the efficient and rapid, small-scale isolation and purification of plant mitochondria. It was also concluded that the fusion polypeptide was capable of being embedded within the outer membrane of plant mitochondria after expression of the genetic construct in the plant cells, and that the N-terminal TwinStrep epitope tag was accessible to streptavidin-coated magnetic beads.

When the isolated and purified mitochondria were analysed by proteomics, the samples were highly enriched for mitochondrial proteins, with very low levels of the small subunit of Rubisco. This further confirmed the high degree of enrichment by using the method.

### Example 14. Association of NifS and NifU Polypeptides in Mitochondria of Plant Cells

Nitrogenase components contain several metalloclusters that are essential for function. The nitrogenase protein for the molybdenum-based enzyme that performs the catalysis, also known as the molybdenum-iron protein, is an α₂β₂-tetramer of the NifD and NifK polypeptides. In the active state, the catalytic tetramer contains a [Fe₈S₇] complex, referred to as a P-cluster, at each a/13 subunit interface and also a FeMo-cofactor (FeMo-co) within each a subunit. The nitrogenase reductase component, also known as the iron protein, is a homodimer of NifH polypeptides which contains a subunit-bridging [Fe₄S₄] cluster. These Fe—S and P-clusters as well as the FeMoco are essential for transfer of electrons for the reduction of N₂. The synthesis and structure of nitrogenase is reviewed in Rubio and Ludden (2005).

The correct assembly and maturation of these metalloclusters is a complicated process and involves several accessory proteins (Rubio and Ludden, 2008). The first step of the maturation process is the generation of basic Fe—S clusters. This is catalyzed by NifS and NifU. In bacteria, these two proteins are required for full nitrogenase activity. The Fe—S clusters are then transferred to NifH, NifB and possibly NifD-NifK. NifS and NifU are not only involved in the assembly of the Mo-dependent nitrogenase, but also in the assembly of VFe and FeFe nitrogenase for synthesis of their Fe—S metalloclusters (Kennedy and Dean, 1992).

These activities have been well studied in bacteria. NifS is a pyridoxal phosphate (PLP, vitamin B6) dependent cysteine desulfurase which generates the inorganic sulphide required for Fe—S cluster synthesis from cysteine. The reaction produces alanine as a byproduct. The sulphide is then provided to NifU for the sequential formation of [Fe₂S₂] and [Fe₄S₄] clusters. The NifS enzyme functions in bacteria as a homodimer.

NifU provides a scaffold for [Fe₄S₄] cluster formation, functioning as a homodimer in bacteria. Its N-terminal domain can bind one [Fe2S2] cluster per monomer. The [Fe₂S₂] clusters in the monomers can be reductively fused to form one [Fe₄S₄] cluster per NifU dimer. The C-terminal domain of NifU can hold one [Fe₄S₄] cluster per monomer. NifU then donates [Fe₄S₄] clusters to NifB for processing into an 8Fe core on NifB, which is subsequently used for the synthesis of FeMoco. In a divergent pathway for the Fe—S clusters, one [Fe₄S₄] cluster bound to the N-terminal or C-terminal scaffolding domains of NifU is transferred to apo-NifH for maturation of nitrogenase reductase, the NifH protein (Smith et al., 2005). It has been proposed that NifU also donates two [Fe₄S₄] clusters to the NifD-NifK and that NifH condenses that pair of clusters into a mature P-cluster [Fe₈—S_{7] (Dos Santos et al.,} 2004).

It has been reported that NifS and NifU form a transient complex in bacteria, but not a tight complex (Yuvaniyama et al., 2000). NifU did not co-purify with NifS when NifS was purified from crude extracts prepared from A. vinelandii (Dos Santos et al., 2012). Furthermore, specific immunoprecipitation of either NifU or NifS did not result in co-precipitation of the other polypeptide. However, when isolated and purified NifU and NifS were combined in vitro and the mixture subjected to size exclusion chromatography, a heterotetrameric complex was detected. However, that experiment used purified proteins. No one has reported co-expressing NifS and NifU in plant cells and showing that they bind to each other, and NifS has not previously been co-purified with NifU from crude extracts.

As described in Examples 2-4, a NifU fusion polypeptide targeted to mitochondria was processed efficiently and accurately by MPP and a NifS fusion polypeptide was processed partially when produced from the genetic constructs SN32 and SN31, respectively. The fusion polypeptides had the MTP-FAγ51 for the mitochondrial targeting and a C-terminal HA epitope for detection by Western blotting. In one experiment, at least 90% of the processed NifU polypeptide accumulated in a soluble form in the plant mitochondria, although the amount varied somewhat from experiment to experiment, and some (<50%) of the processed NifS polypeptide accumulated in a soluble form (FIG. 3). Moreover, NifS and NifU polypeptides that retained the FAγ-scar9 motif at the N-terminus were demonstrated to be functional in E. coli for supporting nitrogenase activity, so both NifS and NifU remained active with a 9-amino acid N-terminal extension (Example 4).

Based on these successes, the inventors designed and carried out further experiments to test the production, processing, solubility and function of NifS and NifU when introduced into mitochondria in plant cells, as follows.

Construction of Plasmids Encoding Fusion Polypeptides with TwinStrep Epitopes.

Two genetic constructs were designed and made for expression of the encoded fusion polypeptides in plant cells, with mitochondrial targeting, one encoding a MTP-FAγ51::NifU::TwinStrep fusion polypeptide (SEQ ID NO:160) and the other a MTP-FAγ51::NifS::TwinStrep fusion polypeptide (SEQ ID NO:161). The amino acid sequences of the NifS and NifU regions of the fusion polypeptides were based on the amino acid sequences of the Klebsiella oxytoca proteins. The TwinStrep epitope (or tag) is abbreviated herein as “TS”. The TwinStrep epitope was chosen as it has a high affinity for binding to StrepTactinXT resin under essentially physiological conditions and was thus ideally suited to the purification of proteins comprising the epitope, even at low concentrations. Furthermore, the elution conditions were gentle, which allowed the purification of protein complexes. The nucleotide sequences of the protein coding regions were codon optimized for improved expression in plant cells. Each genetic construct contained a 35S CaMV promoter sequence (Accession No. EC51288) for expression in the plant cells and a region coding for the 51 amino acids of MTP-FAγ51 fused 5′ to the Nif coding region. These constructs were made using the GoldenGate assembly strategy (Weber et al., 2011; Engler et al., 2014), using analogous methods to those described above. These constructs were designated as SN166 for NifU and SN231 for NifS.

Another construct was made (SN167) which was the same as SN166 except that the MTP-FAγ51 region was mutated so that the encoded fusion polypeptide had alanine substitutions in the MTP sequence that would not allow for processing by MPP in mitochondria—the mutated region was designated mFAγ51.

Production of the Fusion Polypeptides in Plant Cells, their Processing and Solubility

These genetic constructs along with others were tested for production and processing of the encoded polypeptides in plant cells and their solubility. As described in Examples 2 and 3, the construct SN31 encoding the MTP-FAγ51::NifS::HA fusion polypeptide was infiltrated into N. benthamiana leaves and protein extracts analysed by Western blotting with anti-HA antibody. Two polypeptide bands were observed on the blots. These corresponded in size with the unprocessed and MPP-processed polypeptides (Example 3). The processed and unprocessed NifS polypeptides were present in both the soluble and the insoluble protein fractions, indicating partial solubility. In contrast, when SN166 was introduced separately into N. benthamiana leaves, the MTP-FAγ51::NifU::TS fusion polypeptide was efficiently processed by MPP and the resultant scar9-NifU::TS polypeptide was almost fully soluble, where the scar9 included the Gly-Gly linker resulting from the cloning procedure used. As described in Example 4, NifS and NifU polypeptides having a N-terminal extension of 9 amino acids were active in providing nitrogenase function to E. coli when combined with the wild-type K. oxytoca proteins for the other Nifs. It has also been shown that a His-tag on the C-terminus of NifS in A. vinelandii does not interfere with diazotrophic growth and assembly of the FeS clusters on NifH (Smith et al., 2005).

The genetic constructs SN166 and SN167 were introduced separately into N. benthamiana leaves to confirm the effectiveness of the MTP sequence in the SN166-encoded polypeptide and the effects of the mitochondrial targeting on solubility and purification on a StrepTactinXT column. Proteins were extracted from the leaf tissues under non-denaturing conditions. The extraction buffer contained 100 mM Tris-HCl pH 8.0, 150 mM NaCl, 5% (v/v) glycerol, 2 mM TCEP, 1% (w/v) PVP (average MW 40 kDa) and 0.1% Tween 20. A 2 ml StrepTactinXT column was washed with buffer containing 100 mM Tris pH 8.0, 150 mM NaCl and 2 mM TCEP (wash buffer) and then loaded with the protein extract from SN166 or, separately, from SN167. After washing the column to remove unbound proteins, the bound proteins were eluted with wash buffer containing 50 mM biotin. Samples containing protein were concentrated to a volume of 200-500 μL using a 4 mL Amicon Ultra 10 kD MWCO concentrator. Aliquots of 20 μL were subjected to SDS-PAG electrophoresis and Western blotting using the antibody Streptactin HRP. Duplicate gels were stained with Coomassie blue to stain proteins.

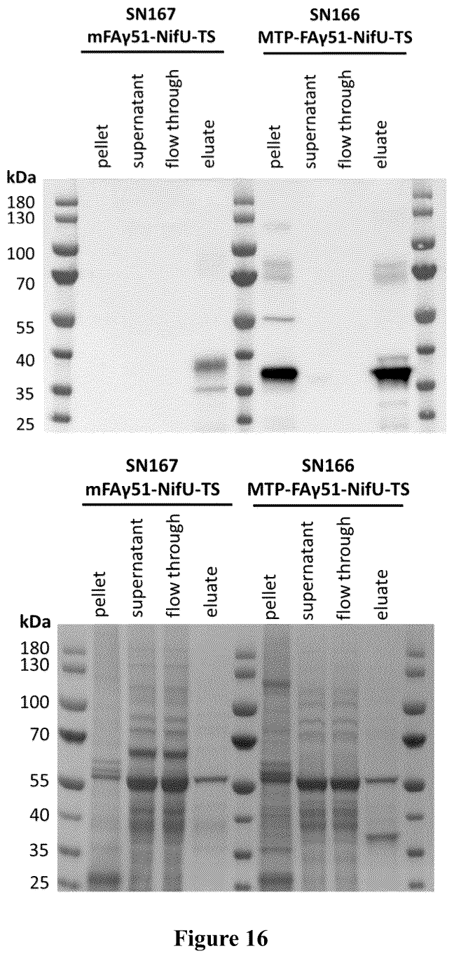

The Western blots (FIG. 16, upper panel) showed that the NifU::TwinStrep fusion polypeptide had indeed been purified from the SN166 infiltrated tissues through the use of the StrepTactinXT column. The extract from the SN167 infiltrated tissues yielded a small amount of purified NifU::TwinStrep protein which appeared to be mostly of the unprocessed form. The corresponding gels stained with Coomassie blue (FIG. 16, lower panel) confirmed that a high degree of enrichment had occurred in the purification process.

Gel slices were cut out of the Coomassie stained gel and the polypeptides in those slices subjected to N-terminal amino acid analysis. This confirmed that the MTP-FAγ51::NifU::TwinStrep fusion polypeptide encoded by SN166 had been cleaved by MPP at the intended site in the MTP sequence, since the purified polypeptide had the N-terminal sequence resulting from the intended processing.

It was concluded from these data that the NifS and NifU fusion polypeptides that were targeted to the mitochondria were indeed expressed in the plant cells and processed in the mitochondria and were sufficiently in a soluble form to allow for purification.

### Co-Expression of MTP::NifU::TwinStrep and MTP::NifS::HA in Plant Cells—NUS and NifU Associate in Plant Mitochondria

To assess the expression, processing, solubility and stability, and to test for possible association of NifS and NifU fusion polypeptides when produced together in plant mitochondria, the genetic constructs SN31 (Example 2) encoding the MTP-FAγ51::NifS::HA polypeptide and SN166 encoding the MTP-FAγ51::NifU::TS polypeptide were co-infiltrated into N. benthamiana leaves using the method as described in Example 1. Protein extracts from the leaves were prepared and examined for the presence of NifS-NifU complexes by, first of all, performing affinity purification of NifU using a StrepTactinXT column and then testing for the co-purification of NifS polypeptide, using the method described in Example 1. Briefly, in a first experiment 12 g fresh weight of leaf material was processed under anaerobic conditions using an extraction buffer which was non-denaturing. A second, repeat purification started with 16.6 g fresh weight of leaf material. A third purification was carried out using 23 g fresh weight leaf material, where the buffer used was slightly different in that Fe′ and L-cysteine were added to 2 mM and 0.5 mM, respectively. In each experiment, the filtered lysate was passed through a StreptactinXT column (IBA Lifesciences) to retain the NifU polypeptide by its TS epitope. After washing the column, bound proteins were eluted with a buffer containing biotin and then concentrated as described above. Samples were retained at each step of the purification process, specifically samples from: (i) the total extractable protein at the start of the experiment, (ii) the pelleted cell debris after the first centrifugation, (iii) the input protein solution which was the fraction soluble in extraction buffer prior to passage over the column, (iv) the flowthrough fraction which did not bind to the column and (v) the concentrated eluate after elution with biotin. Samples were treated with SDS and heating to 95° C. before SDS-PAG electrophoresis and Western blotting.

The purified and concentrated NifU sample from the third purification contained some visible brown colour, indicating the presence of Fe—S clusters.

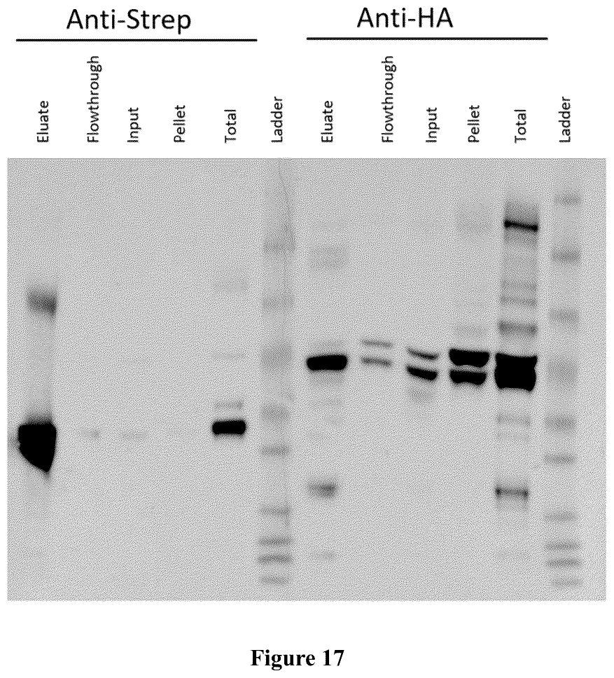

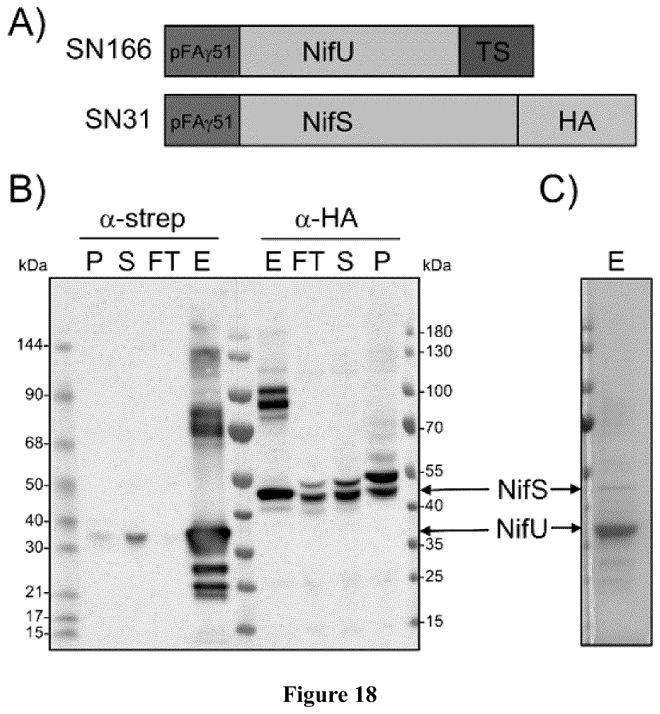

Duplicate aliquots of these samples were subjected to Western blot analysis with immunodetection using anti-Strep antibody or anti-HA antibody. Western blots from the first and third purification experiments are shown in FIGS. 17 and 18. The third purification experiment was done in the presence of 0.5 mM L-cysteine and 2 mM Fe²⁺ supplementation in the extraction buffer. The Western analysis showed that both proteins were present in the soluble fraction after extraction from the leaf material. For NifS, both the processed and the unprocessed forms were present in the soluble fraction, while for NifU only the processed form was present, indicating efficient processing. The anti-Strep antibody detected a scar9-NifU-TwinStrep polypeptide in the crude samples as well as in the sample eluted from the column. The intensity of the signal from the eluate was very strong, indicating that the scar9-NifU-TwinStrep polypeptide had been purified and concentrated from the plant extracts. The mobility of the polypeptide in the gels upon electrophoresis was consistent with mitochondrial processing within the MTP sequence and the processing appeared to be almost complete.

When the membrane was exposed to the anti-HA antibody, which was approximately 20× more sensitive than the anti-strep antibody, a HA-tagged polypeptide was revealed at a size consistent with mitochondrial processing of the NifS polypeptide, i.e. scar9-NifS::HA. No unprocessed form of the NifS fusion polypeptide was detected in the sample. Since the NifS polypeptide used in this experiment did not contain a strep-tag, these results indicated that NifS and NifU formed a complex and that the NifS polypeptide was co-purified through its interaction with NifU. Significantly, it was observed that the processed form, scar9-NifS::HA, was greatly enriched in the eluate from the column relative to the unprocessed form when compared to the ratio of the two forms in the input sample prior to column purification. These observations were surprising to the inventors on the basis of reports from bacteria expressing NifS and NifU which had not demonstrated association of the polypeptides. They concluded that, under the anaerobic, non-denaturing conditions used in the experiment for protein extraction: (i) the NifS fusion polypeptide was co-purified with the scar9-NifU::TS polypeptide, indicating an association of the two polypeptides when co-expressed in the plant cells with mitochondrial targeting, (ii) the MPP-processed form of the NifS polypeptide, scar9-NifS::HA was the form that associated with the NifU polypeptide, and (iii) that both the processed NifU and the processed NifS polypeptides were produced in at least partly soluble form in the mitochondria to allow for the observed association. There were at least three possible explanations for observation (ii). Firstly, unprocessed MTP-FAγ51::NifS might not have been able to interact with NifU due to steric hindrance or misfolding. Secondly, the unprocessed form might not have been imported into the mitochondria where the NifU polypeptide was localized, and thirdly, the unprocessed form of NifS might not have been sufficiently soluble and was thus not able to interact with NifU, or any combination of these reasons.

The inventors were not aware of any previous reports of a NifS-NifU complex being isolated from plant mitochondria, or indeed from any cell.

The samples from the first purification were again subjected to denaturing SDS-PAGE. This time, the gels were stained with Coomassie blue (FIG. 18, panel C) and regions of the gel corresponding to the processed NifU and NifS polypeptides were analysed by proteomics to identify both the introduced polypeptides and any endogenous proteins that were co-purified on the column. The gel slices were treated as described in Example 1, including with trypsin digestion and analysed by LC-MS/MS. The analysis identified the presence of peptide ISTQVVR (SEQ ID NO:119) predicted for tryptic digestion of the scar peptide (SEQ ID NO:42) at the N-terminus, showing that both NifS and NifU were processed exactly at the predicted MPP-cleavage site within the MTP. Targeted MRM confirmed the identity of the tryptic peptides and thereby confirmed the presence of the cleaved polypeptides at the regions expected in the SDS-PAGE gel.

### Size Exclusion Chromatography

To further confirm that a protein complex was formed between NifS and NifU, a sample of the concentrated eluate was applied to a high-resolution size exclusion chromatography using as a Superdex 200 Increase 3.2/300 column. Calibration of the column was carried out with native protein size markers (Biorad Gel Filtration Standard Cat. #151-1901). Fractions from the column were further analysed by electrophoresing samples on denaturing SDS-PAGE. The chromatogram and Western blot analysis showed that NifS and NifU formed a complex, as the NifS protein eluted at a higher molecular weight than expected for NifS. This indicated that a heterotetramer formed by association of 2 NifS and 2 NifU polypeptides.

### UV/Visible Spectroscopy Detected Iron-Sulfur Clusters on NifU

Eluate containing StreptactinXT column-purified NifU and NifS from a fourth experiment was applied to a PD10 column (GE Healthcare) equilibrated in 50 mM Tris-HCl pH 8.0 and 300 mM NaCl to remove biotin and excess Fe²⁺ and cysteine. A spectrum was obtained using an anaerobic cuvette with a screw cap and septum with a 1 cm pathway on a Cary 100 Bio UV/visible spectrophotometer. The spectrum showed one main peak at 280 nm, as expected for proteins due to the absorption from tryptophan, phenylalanine and cysteine. Additionally, a second peak was observed at 325 nm and a shoulder at 420 nm and 460 nm, which indicated the presence of Fe—S clusters on NifU.

### Further Tests for Association of NifS and NifU Polypeptides by Purification First of all of NifS.

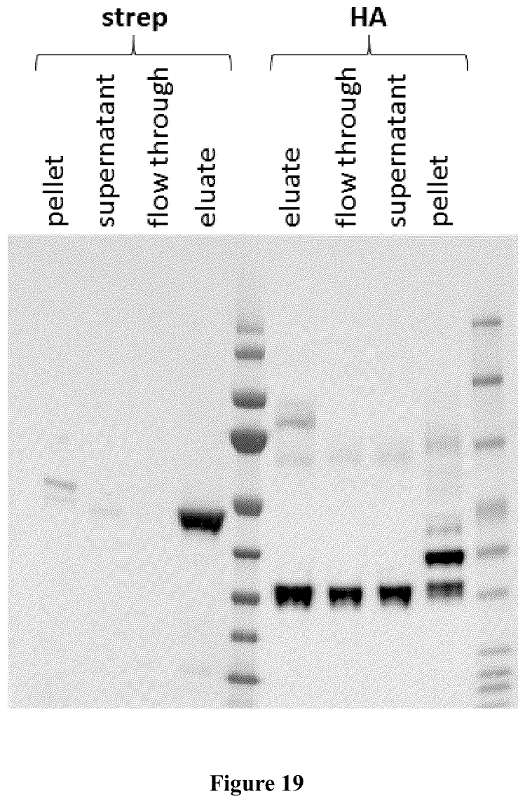

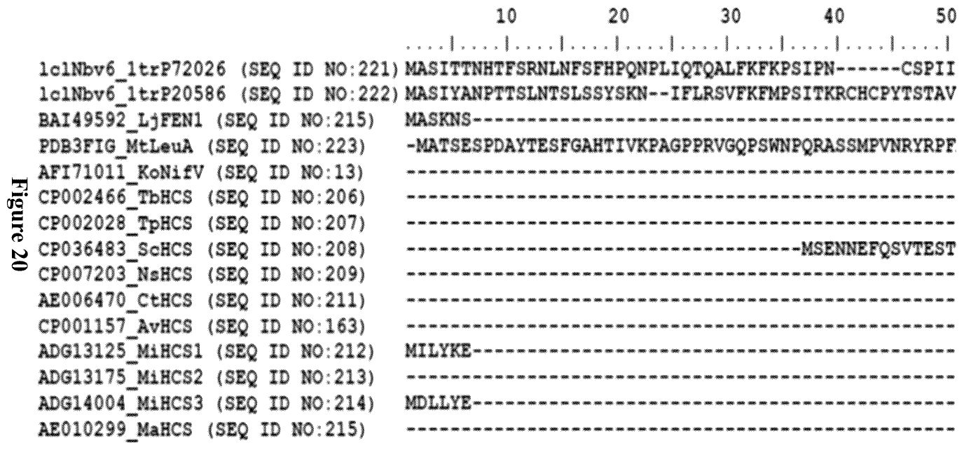

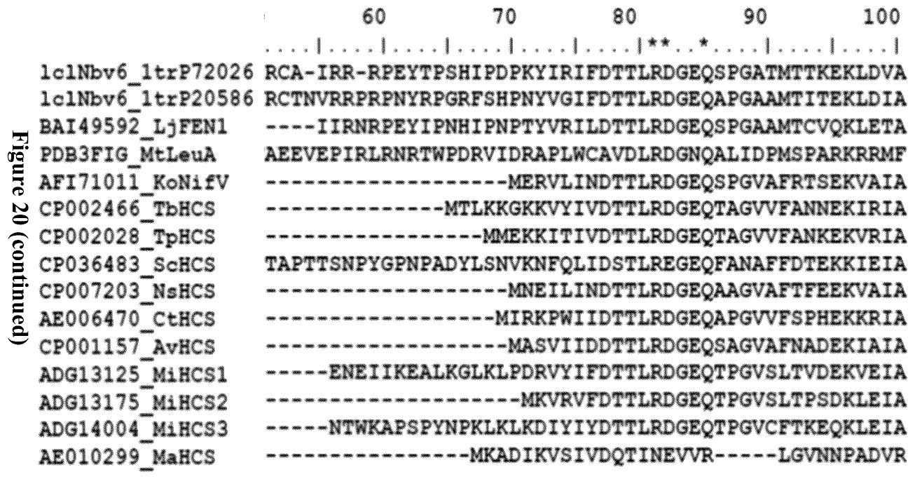

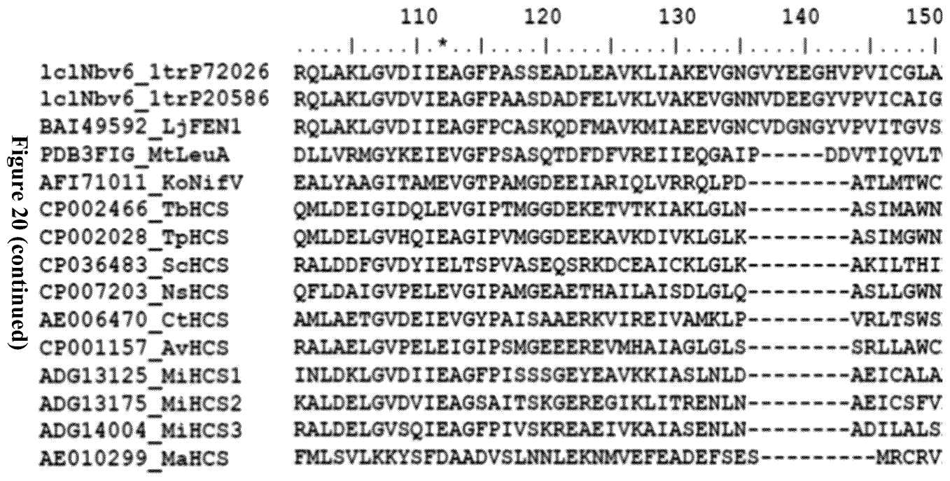

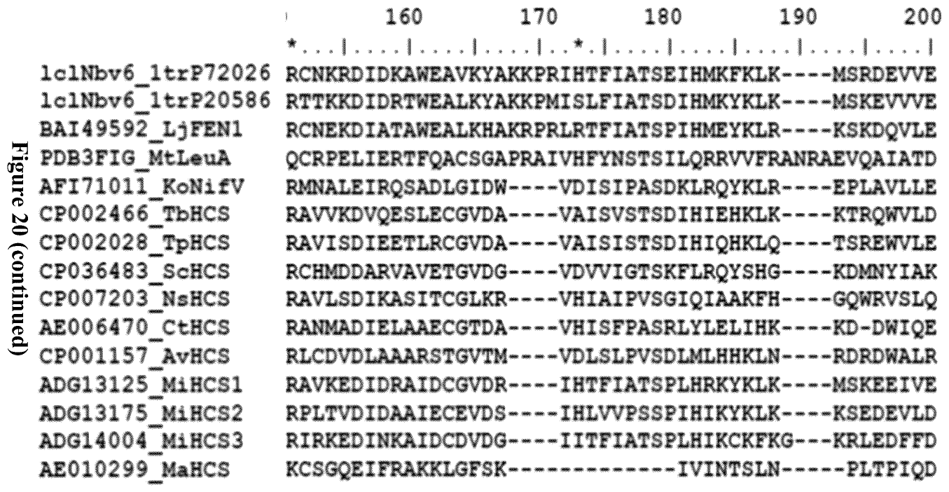

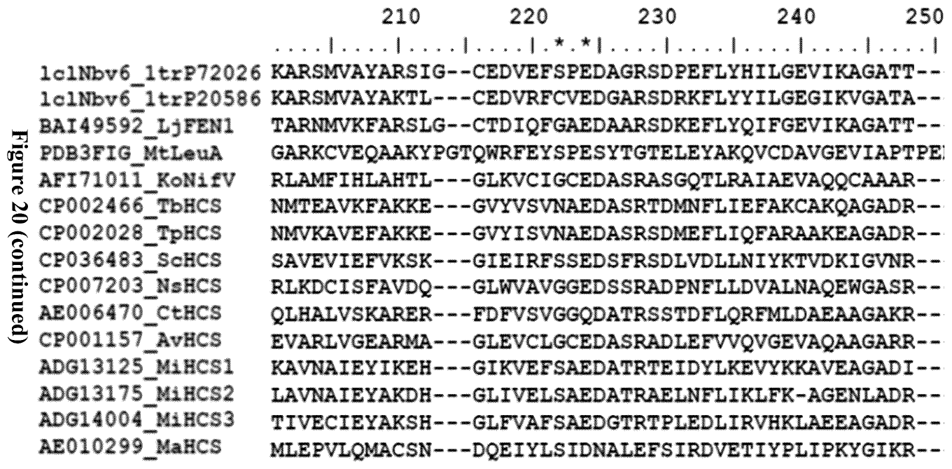

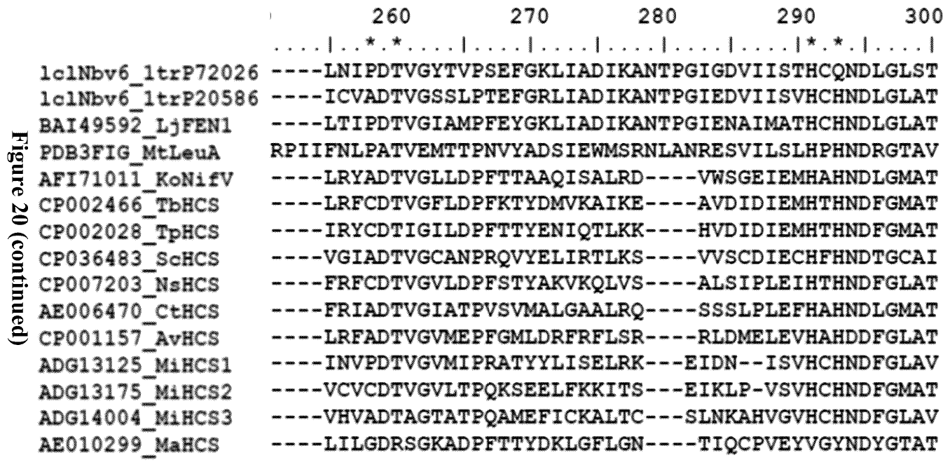

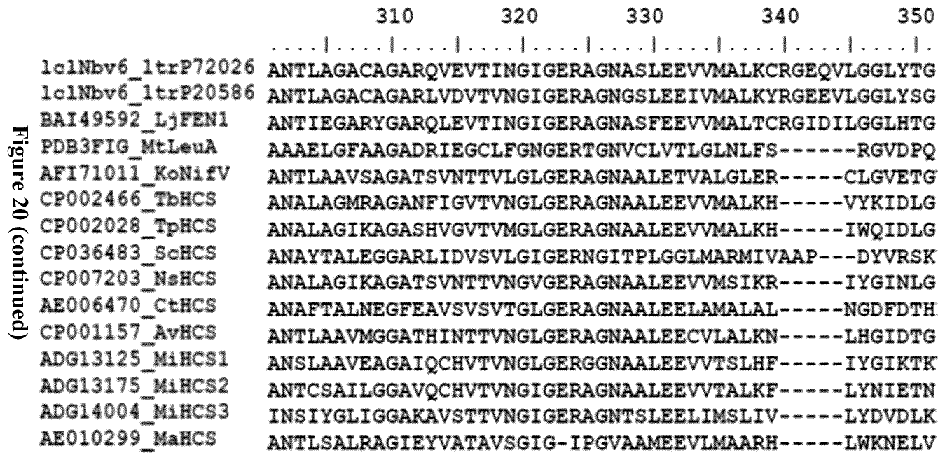

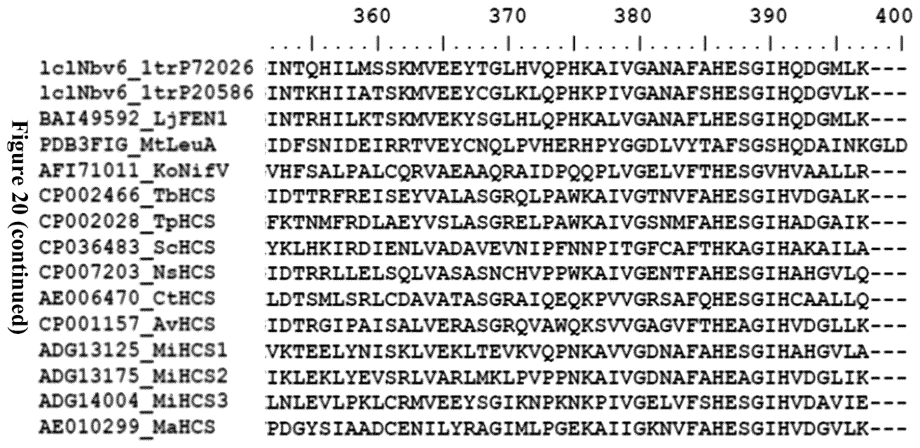

As described above, a genetic construct was designed and made that encoded a MTP-FAγ51::NifS::TS fusion polypeptide, designated SN231, for transient expression in plant cells. This construct was analogous to SN166 that encoded the MTP-FAγ51::NifU::TS fusion polypeptide except that it had the NifS sequence rather than the NifU sequence. SN231 and SN32 were co-infiltrated into N. benthamiana leaves as for the SN31/SN166 combination described above, and protein extracts are prepared as described above. The supernatant was passed through a StrepTactinXT column to purify NifS fusion polypeptide containing the TwinStrep epitope. Samples of the eluted and concentrated proteins were analysed by Western blotting and are probed with anti-Strep and anti-HA antibodies. The blot (FIG. 19) showed the presence of a processed scar9::NifU::HA polypeptide in the eluate as well as the scar9::NifS::TS polypeptide, again indicating the association of processed NifS and NifU polypeptides in the extracts from the plant cells.

The eluate from this purification was also subjected to size exclusion chromatography as described above, and the fractions were analysed by Western blot using anti-strep antibody and anti-HA antibody. The Western blot analysis confirmed that NifU and NifS formed a complex.

In the future, the purified NifS and NifU polypeptides will be analysed by Inductively Coupled Plasma Mass Spectrometry (ICP-MS) to determine the iron content of the protein, and with Mossbauer spectroscopy to confirm the presence and the type and redox status of Fe—S clusters bound to the polypeptides.

Cluster formation can be shown in in vitro reactions with added Fe²⁺ and L-cysteine. In one experiment, wild-type NifH polypeptide is purified from A. vinelandii and the Fe—S clusters removed by chelation to produce apo-NifH polypeptide. Wild-type NifD-NifK complex is also purified from A. vinelandii. In vitro ARA assays show that the purified NifU polypeptide purified from N. benthamiana cells as described above is able to donate Fe—S clusters to the apo-NifH polypeptide, thereby reconstituting NifH activity as the nitrogenase reductase for ARA activity.

### Example 15. Production of Homocitrate by Expression of NifV in Plant Cells

Introduction (R)-2-hydroxy-1,2,4-butane-tricarboxylic acid, referred to herein and commonly known as homocitrate, is required for the activity of all known nitrogenases, namely the molybdenum (Mo—Fe), vanadium (V—Fe) and iron (Fe—Fe) nitrogenases respectively (Hu and Ribbe, 2016). The nitrogenase protein for the Mo-based enzyme that performs the reduction of nitrogen is an α₂β₂-tetramer of the NifD and NifK polypeptides that contains a FeMo cofactor (FeMoco) within each a subunit as well as a [Fe₈S_{7] complex, referred to as a P-cluster, at each a/}13 subunit interface. The FeMoco which comprises a homocitrate molecule is essential for the reduction of N₂.

Homocitrate (HC) forms part of the essential nitrogenase cofactors FeMoco, FeVco and FeFeco in bacteria expressing nitrogenase, binding to the Mo, V or Fe atoms of the cofactor through its 2-hydroxy and 2-carboxy groups. FeMoco, FeVco and FeFeco are at the sites of catalysis and the three cofactors are thought to bind, activate, and reduce N₂ in largely the same way. FeMoco, also known as the M-cluster of the Mo-nitrogenase, contains [Fe₄S₃] and [MoFe₃S₃] subclusters joined through three bridging inorganic sulfide atoms referred to as “belt sulphides” and one interstitial carbide atom (Hu and Ribbe, 2016) to form the cofactor having the chemical formula HC—Mo—Fe₇—S₉—C. The vanadium-nitrogenase including its cofactor FeVco has recently been crystallised (Sippel and Einsle, 2017; Sippel et al., 2018). FeVco has a nearly identical metal-sulfur core to FeMoco except for the substitution of a vanadium atom for the molybdenum atom and a carbonate ion instead of one of the belt sulfides. FeVco is therefore a [HC—V—Fe₇—S₈CO₃—C] cluster with the homocitrate molecule liganded to the vanadium atom. In the case of the Azotobacter vinelandii VnfD polypeptide that is part of the catalytic V-nitrogenase enzyme (VnfDGK), the homocitrate of the metallocluster is coordinated to amino acids C257 and H423 of VnfD. These ligand amino acids are highly conserved relative to NifD of the Mo-nitrogenase. The Mo- and V-nitrogenases differ in reactivity to carbon monoxide (CO) which inhibits the former but is converted to hydrocarbons by the latter (Sippel et al., 2018). Homocitrate similarly forms part of FeFeco and the cofactor binds to the AnfD polypeptide in analogous fashion. The Fe-nitrogenase has lower N₂-reducing activity compared to the V-nitrogenase which in turn is less active than Mo-nitrogenase, suggesting that organisms that are equipped with all three systems resort to a preferential expression that depends on relative Mo, V and Fe bioavailability. For example, the bacterium A. vinelandii can express each of the Mo-, V- and Fe-nitrogenases, but each one under different nutrient conditions, the V-nitrogenase only under molybdenum-limited conditions, and the Fe-nitrogenase only when both Mo and V are limiting.

In free-living nitrogen-fixing bacteria, homocitrate is produced by the NifV gene product, an enzyme that condenses acetyl-CoA and α-ketoglutarate (αKG) to make the homocitrate (Zheng et al., 1997). NifV is the only gene product required for homocitrate synthesis in these bacteria. The homocitrate synthase activity can be measured by enzyme assays as described in Zheng et al. (1997). A. vinelandii nifV mutants are unable to produce any form of fully-active nitrogenase, but the activity of all three nitrogenases was restored by the addition of homocitrate to the growth medium (Zheng et al., 1997). In the absence of added homocitrate, the mutant nifV bacteria exhibited abnormal nitrogenase-mediated reactions including altered substrate specificity and inhibitor specificity. The mutant bacteria reduced acetylene and evolved Hz, but did not reduce Nz (McLean and Dixon, 1981). These altered activities were due to the incorporation of endogenous molecules related to homocitrate such as citrate within the metallocluster (Hoover et al., 1988). It is thought that homocitrate is unique in its ability to correctly place the substrate N₂ within the active site and is therefore required for fully and properly functional nitrogenases.

The A. vinelandii NifV is the best studied NifV (Zheng et al., 1997; SEQ ID NO:163), referred to herein as AvNifV. Over-expression of the AvNifV polypeptide in E. coli generated a dimeric protein having a molecular weight of approximately 89 kDa, with the monomer having a molecular weight of 44 kDa. The enzyme was oxygen labile, losing approximately 50% of its activity after two hours exposure to air having 21% oxygen. This oxygen sensitivity of its condensing activity was not impacted by addition of MoO₄⁻², Fe²⁺ or Mg²⁺ to the reaction medium. Reaction kinetics indicated that AvNifV had a Km of 0.06 mM for acetyl-CoA and 2.24 mM for αKG. NifV can also condense acetyl-CoA to other keto-acid substrates such as oxaloacetate and α-ketoadipate (Zheng et al., 1997).

In legume-rhizobia symbioses such as between Lotus japonicus and Mesorhizobium loll, the bacterial partner does not have homocitrate synthase activity encoded by a NifV gene. Instead, the host plant L. japonicus expresses a homocitrate synthase, LjFEN1, to supply this essential organic acid for nitrogen fixation by the rhizobia in the nodules (Hakoyama et al., 2009). The LjFEN1 polypeptide is rather distantly related to A. vinelandii NifV, the two polypeptides having about 36% amino acid identity. LjFEN1 has 540 amino acid residues and a molecular weight of approximately 58.6 kDa. No signal peptide sequence was found in the gene encoding LjFEN1, indicating that it was probably a cytosolic protein. L. japonicus has two orthologues of FEN1, namely Accession Nos. AK339695 and AK339656, which are 81% and 71% identical in amino acid sequence to LjFEN1, respectively. Phylogenetic analysis suggested LjFEN1 evolved from AK339695. In symbioses between L. japonicus plants mutated in LjFEN1 and M. loti, fully functional nodules with detectable nitrogenase activity were produced if the microsymbiont carried a heterologous copy of the AvNif17 or the FEN1 gene.

In contrast to many other eukaryotes, fungi such as the yeast Saccharomyces cerevisiae produce homocitrate as an intermediate in the lysine biosynthesis pathway through a NifV-like enzyme (Thomas et al., 1966; Verhasselt et al., 1995). Yeast mutants in the gene ORF D1298 encoding the NifV-like enzyme which functions in that pathway were complemented by over-expression of LjFEN1.

Genomic analyses of numerous plant species indicate that only those plants involved in symbiotic relationships with bacteria express an active homocitrate synthase, such as LjFEN1, and that NifV-like genes are not found non-leguminous plants (Hakoyama et al., 2009). Additionally, no metabolic pathway has been identified in higher plants for synthesis of lysine through homocitrate as an intermediate. Consistent with these reports, examination of the genome sequence of N. benthamiana (Naim et al., 2012) did not identify any homologues of NifV or FEN1. The closest gene identified in terms of homology was a gene (QUT N. benthamiana Genome and Transcriptome DB Accession No. P72026) that was homologous to a gene encoding the enzyme 2-isopropylmalate synthase (EC. 2.3.3.13) involved in leucine biosynthesis but not in homocitrate synthesis. The inventors concluded that N. benthamiana did not normally produce homocitrate by NifV or FEN1-like enzymes. Other than a single report related to vanilla pods (Palama et al., 2009), the inventors are not aware of any report of homocitrate being produced in non-legumes, including no reports for tobacco, cotton and cereals. There are no known reports of FEN1 or NifV being used to produce homocitrate in non-legumes.

### Results

As described in Examples 2-4 above, a NifV fusion polypeptide based on the K. oxytoca amino acid sequence (KoNifV; SEQ ID NO:13) and targeted to mitochondria in plant cells was processed efficiently (>90%) and accurately by MPP when produced from the genetic construct SN142. The fusion polypeptide as translated upon expression of the genetic construct had an N-terminal MTP-FAγ51 for mitochondrial targeting and a C-terminal HA epitope for detection by Western blotting. Moreover, the NifV polypeptide based on the K. oxytoca amino acid sequence with the FAγ-scar9 motif fused at the N-terminus was demonstrated to be functional in E. coli for supporting near wild-type levels of nitrogenase activity, providing about 90% of activity in the MIT2.1 system relative to wild-type, so the NifV fusion polypeptide remained active with a 9-amino acid N-terminal extension (Example 4). However, the processed KoNifV polypeptide accumulated in an insoluble form in the plant mitochondria (FIG. 3).

The insolubility of the K. oxytoca NifV fusion polypeptide in N. benthamiana cells was considered by the inventors to be a problem for constituting nitrogenase function in plant cells since an essentially insoluble polypeptide was unlikely to provide sufficient enzymatic function for the synthesis of homocitrate. Therefore, the inventors sought more soluble NifV polypeptides by expression of natural NifV and other HCS-like variants fused to the same MTP and HA epitope sequences for mitochondrial localisation and detection.

### Selection of Variant NifV Sequences

Sequence databases were searched for NifV variant sequences and other homocitrate synthase (HCS) enzymes related to the KoNifV amino acid sequence. The sequences were from a wide variety of bacteria and yeasts, including some from thermo-tolerant bacteria. NifV polypeptide sequences were extracted from the UniProt database using NifV as the query, accessing the database on 14/09/2018. 2044 NifV/HCS-like amino acid sequences were identified and extracted from the database. To select and test representative sequences, a protein network was established based on protein similarity, resulting in the clustering of NifV?HCS-like polypeptides based on sequence similarity.

To do this, the amino acid sequences were aligned with MAFFT—Multiple alignment program for amino acid or nucleotide sequences—software, version 7 using the server mafft.cbrc.jp/alignment/server/large.html?aug31. The strategy G-large-INS-1 for less than 10000 sequences, shorter than 5000 sites, was used. The output was converted from .pir to .phy format using an online sequence converter (www.hiv.lanl.gov/content/sequence/FORMAT_CONVERSION/form.html).

Cytoscape (https://cytoscape.org) software was used to visualize clusters of sequences that were related to each other. In order to calculate distance matrices and prepare the data in the input files for Cytoscape, the PHYLIP/protdist program was used to calculate the Kimura distance matrix for the NifV sequences. The output file was modified using Notepad to prepare an appropriate input format for the aMATReader in Cytoscape. The distance matrix was then modified in Excel to decrease file size and define subgroups: all values that were greater than 0.1 were removed, thereby creating subgroups, redundant sequences were removed.

A representative HCS-like amino acid sequence was selected from each of six clusters of HCS and related sequences. Additionally, three Methanocaldococcus infernus HCS-like sequences were selected because they were considered more likely to be thermotolerant and possibly remain stable and soluble, as well as NifV sequences from K. oxytoca (KoNifV) and A. vinelandii (AvNifV) as comparisons. A variant of KoNifV (Accession No. WP_004138778; SEQ ID NO:164) was also identified, based on the amino acid sequence in the bacterial expression construct MIT2.1. The amino acid sequences of KoNifV in EC38020 and NifV in MIT2.1 differed in amino acids 155-157 and 232-236 relative to SEQ ID NO:13 but were otherwise identical. A Saccharomyces cerevisiae HCS (ScHCS) sequence was also selected, corresponding to the S. cerevisiae gene Lys21p, referred to as D1298 in Verhasselt et al. (1995). A homologous enzyme in S. cerevisiae, Lys20, appeared to be more active and less negatively regulated by lysine.

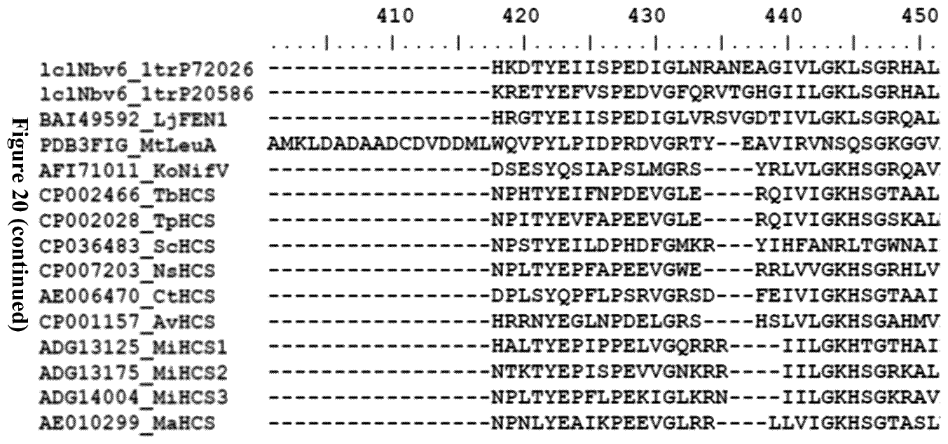

The selected sequences are listed in Table 15 along with the percentage identity to KoNifV from EC38020 (SEQ ID NO:13). A sequence alignment for the amino acid sequences is shown as FIG. 20, which shows highly conserved amino acids. Clearly the selected sequences covered a wide range of NifV/HCS-like sequences.

TABLE 15
NifV and NifV/HCS-like sequences selected for testing.

| | | | Sequence | |
|---|---|---|---|---|
| Sequence | Genbank | | identity | SEQ ID |
| ID | accession | Organism | (%) | NO |
| KoNifV | AFI71011 | Klebsiella oxytoca | 100 | 13 |
| KoNifV | WP_004138778 | Klebsiella oxytoca, variant | 98 | 164 |
| (MIT2.1) | | NifV in pMITv2.1 | | |
| AvNifV | CP001157 | Azotobacter vinelandii | 42 | 163 |
| TbHCS | CP002466 | Thermoanaerobacter brockii | 56 | 206 |
| TpHCS | CP002028 | Thermincola potens | 65 | 207 |
| ScHCS | CP036483 | Saccharomyces cerevisiae | 43 | 208 |
| NsHCS | CP007203 | Nodularia spumigena | 43 | 209 |
| MaHCS | AE010299 | Methanosarcina acetivorans | 55 | 210 |
| CtHCS | AE006470 | Chlorobaculum tepidum | 27 | 211 |
| MiHCS1 | ADG13125 | Methanocaldococcus infernus | 44 | 212 |
| MiHCS2 | ADG13175 | Methanocaldococcus infernus | 42 | 213 |
| MiHCS3 | ADG14004 | Methanocaldococcus infernus | 40 | 214 |
| LjFEN1 | BAI49592 | Lotus japonicus | 30 | 215 |

Construction of Plasmids Encoding Fusion Polypeptides with NifV and NifV/HCS-Like Sequences.

Fusion polypeptides having the selected NifV and NifV/HC S-like sequences, listed in Table 15, and having MTP-FAγ51 at each N-terminus were then tested for their ability to be expressed in plant cells, their processing by MPP in the mitochondria and their production of homocitrate. The solubility of each mitochondrially-targeted polypeptide was also tested using the method described in Example 1. These experiments were done by generating genetic constructs encoding these sequences and expressing them in the N. benthamiana leaf system. Each encoded fusion polypeptide had an identical HA epitope for detection with anti-HA antibody, located between the MTP and NifV/HCS-like sequence, except for the KoNifV fusion polypeptide encoded by SN142 which had the HA epitope at its C-terminus (Table 15). This experiment was therefore designed to test whether the N- or C-terminal extensions to each NifV/HCS-like sequence would still allow for production of homocitrate in the plant cells. A parallel set of genetic constructs (Table 15) was made to express cytoplasmically-targeted polypeptides lacking the MTP-FAγ51 sequence at the N-termini but instead having a N-terminal HA epitope. Each fusion polypeptide was thereby compared for its expression and function to its corresponding cytoplasmic polypeptide lacking the MTP sequence.

A DNA sequence for each fusion polypeptide was synthesised using codon optimisation for plant expression and compatible with GoldenGate cloning protocols. The genetic constructs were made using a modular cloning system by the GoldenGate protocol. Except for SN142, the DNA components were, in the 5′ to 3′ order for assembly: the 35S CaMV promoter (EC51288), a chimeric sequence coding for the MTP-FAγ51 and HA epitope followed by a GG linker (EC38095), a codon-optimised coding regions for the NifV/HCS-like variant, and finally a CaMV 3′ polyadenylation region/transcription terminator (EC41414). The components were assembled into the desired genetic constructs and inserted into expression vectors using Type IIS restriction cloning according to GoldenGate assembly (Weber et al., 2011). The resultant constructs are listed in Table 16. Molecular weights of the encoded fusion polypeptides before and after MPP-processing were calculated using ExPASy compute p1/Mw (web.expasy.org/compute_pi/) with monoisotopic setting.

TABLE 16
Genetic constructs for transient expression of NifV/HCS-
like fusion polypeptides in N. benthamiana leaves or
in stably transformed plants. MTP, FAγ51 mitochondrial
targeting peptide; Mw, molecular weight; na, not applicable.

| | | | | Processed | |
|---|---|---|---|---|---|
| | | Length | | length | |
| Construct | Fusion | (amino | Mw | (amino | Mw |
| ID | polypeptide | acids) | (Da) | acids) | (Da) |
| SN142 | MTP::KoNifV::HA | 445 | 48088 | 403 | 43529 |
| SN248 | MTP::HA::TbHCS | 447 | 49336 | 405 | 44777 |
| SN249 | MTP::HA::TpHCS | 442 | 48521 | 400 | 43963 |
| SN250 | MTP::HA::ScHCS | 504 | 55488 | 462 | 50929 |
| SN251 | MTP::HA::NsHCS | 440 | 47715 | 398 | 43156 |
| SN252 | MTP::HA::MaHCS | 440 | 48316 | 398 | 43758 |
| SN253 | MTP::HA::CtHCS | 440 | 47931 | 398 | 43372 |
| SN254 | MTP::HA::AvNifV | 448 | 48395 | 406 | 43836 |
| SN255 | MTP::HA::MiHCS1 | 575 | 62818 | 533 | 58259 |
| SN256 | MTP::HA::MiHCS2 | 554 | 60106 | 512 | 55548 |
| SN257 | MTP::HA::MiHCS3 | 460 | 50986 | 418 | 46428 |
| Cytoplasmic constructs | | | | | |
| SN212 | KoNifV::HA | 392 | 42362 | na | na |
| SN258 | HA::TbHCS | 395 | 43741 | na | na |
| SN259 | HA::TpHCS | 390 | 42926 | na | na |
| SN260 | HA::ScHCS | 452 | 49892 | na | na |
| SN261 | HA::NsHCS | 388 | 42120 | na | na |
| SN262 | HA::MaHCS | 388 | 42721 | na | na |
| SN263 | HA::CtHCS | 388 | 42336 | na | na |
| SN264 | HA::AvNifV | 396 | 42800 | na | na |
| SN265 | HA::MiHCS1 | 523 | 57223 | na | na |
| SN266 | HA::MiHCS2 | 502 | 54511 | na | na |
| SN267 | HA::MiHCS3 | 408 | 45391 | na | na |
| SN70 | HA::LjFen1 | 552 | 59962 | na | na |

Expression in N. benthamiana Leaves and Testing for Solubility and Homocitrate Production

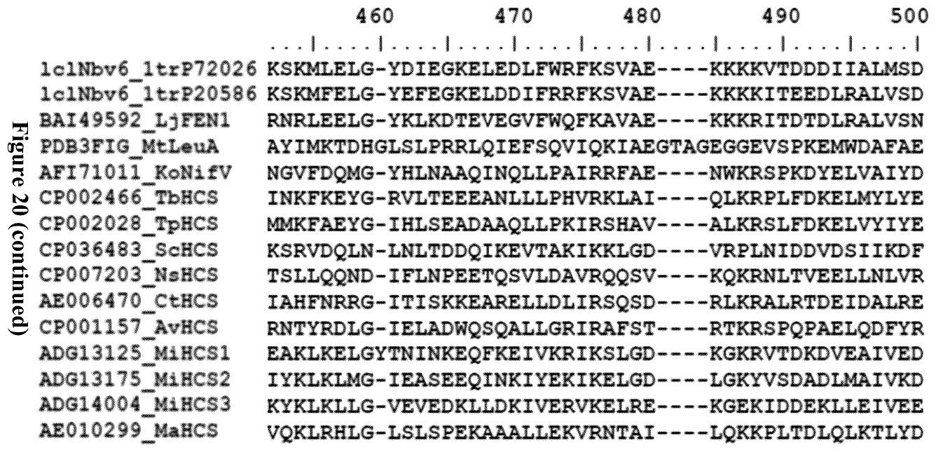

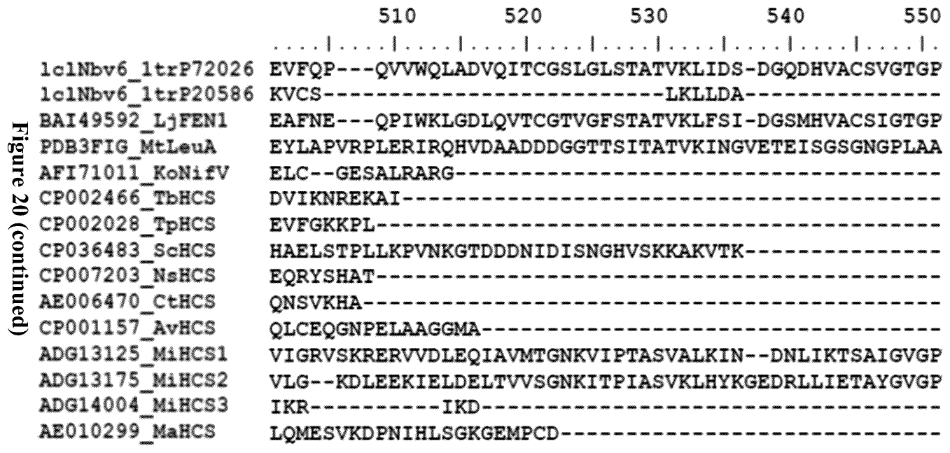

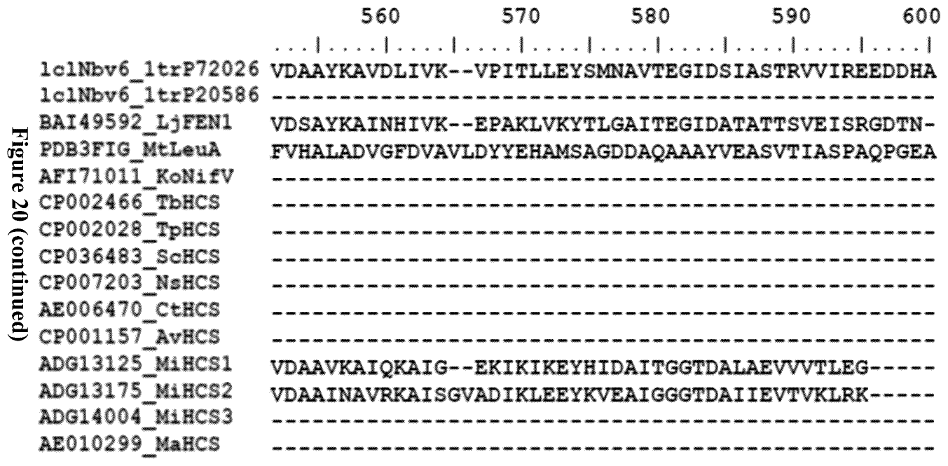

Each genetic construct was introduced into N. benthamiana leaves via Agrobacterium using the methods as described in Example 1. Leaf samples were harvested 5 days post-infiltration and protein extracts made and analysed by Western blot methods using anti-HA antibody (FIGS. 21 and 22). Parallel leaf samples were harvested for metabolite extraction and measurement of homocitrate levels by a GC-MS/MS technique as described below.

All of the tested fusion polypeptides were readily detected by the Western blotting analysis and so were expressed efficiently in the plant cells, for both the mitochondrially-targeted and the cytoplasmically-targeted polypeptides. As observed previously (Example 3), the mitochondrially-targeted K. oxytoca NifV fusion polypeptide was produced at good levels and processed efficiently by MPP but was essentially insoluble in the plant cells. In similar fashion, the mitochondrially-targeted MiHCS2, MiHCS3 and MaHCS fusion polypeptides were also expressed at good levels and appeared to be processed but insoluble. The NsHCS and MiHCS1 fusion polypeptides appeared to be processed but were only partially soluble. In contrast, the TbHCS, TpHCS and CtHCS fusion polypeptides appeared to be processed and essentially soluble when targeted to the mitochondria. The mitochondrially-targeted S. cerevisiae HCS (ScHCS) appeared to be expressed at a lower level than the other polypeptides but was efficiently processed and soluble. The Azotobacter vinelandii NifV (AvNifV) fusion polypeptide was expressed at a good level, efficiently processed and was partly soluble (˜50%) when targeted to plant mitochondria using MTP-FAγ51. Likewise, the Chlorobaculum tepidum HCS (CtHCS) was well expressed, efficiently processed and soluble when targeted to plant mitochondria using MTP-FAγ51.

In contrast to the mitochondrially-targeted polypeptides, most of the cytoplasmically-targeted polypeptides were soluble or at least partially soluble, including the KoNifV polypeptide (FIG. 22). The inventors concluded that the insolubility was due in some cases to the mitochondrial localisation, and that polypeptides could show different levels of solubility in the two locations. In general, the signal intensities of the cytoplasmically targeted polypeptides were lower compared to the corresponding mitochondrially targeted polypeptides. The exceptions were ScHCS, MiHCS1 and KoNifV, where the cytoplasmically targeted polypeptides appeared to have better expression levels compared to the counterparts that were targeted to the mitochondria.

### Gas Chromatography-Tandem Mass Spectrometry (GC-MS/MS) Analysis to Measure Homocitrate Levels

To measure homocitrate levels in the leaf samples after gene introduction and thereby demonstrate HCS activity for either the mitochondrially-targeted or cytoplasmically-targeted fusion polypeptides, a GC-MS/MS method was developed and validated, as follows. Polar metabolites including any homocitrate were extracted into 10 volumes per wet leaf weight (v/w) of extraction solution which contained in methanol:H₂O (1:1 v/v): 22 μM D4 citric acid (Cambridge Isotope Laboratories Inc., cat. no. DLM-3487), 36 μM 13C fumaric acid (Cambridge Isotope Laboratories Inc., cat. no. CLM-1529), 23 μM 13C sorbitol (Cambridge Isotope Laboratories Inc., cat. no. CLM-1565), 31 μM D3 aspartic acid (Cambridge Isotope Laboratories Inc., cat. no. DLM-832), and 54 μM D5 glycine (Cambridge Isotope Laboratories Inc., cat. no. DLM-280) as internal standards. The leaf samples were homogenised with the extraction solution in 1.5 ml microfuge tubes using a Qiagen tissue lyser and 3 mm tungsten carbide beads. The leaf samples were homogenised at 1/20 rpm twice for three minutes while rotating the tube positions within racks that were pre-chilled to −80° C. After homogenisation, the samples were centrifuged at 10,000×g for 30 minutes at 4° C. to remove solid matter, and the resulting supernatant containing the metabolites was collected and stored at ˜80° C. until analysis. Thirty μl of each supernatant was dried in a vacuum concentrator for metabolite derivatization, which was carried out manually as follows. To each dried sample, 10 μl of 20 mg/ml methoxyamine hydrochloride in pyridine was added. The solutions were incubated at 37° C. for 90 min with vortexing at 15 min intervals, then 15 μl of N,O-bis(trimethylsilyl)trifluoroacetamide+trimethylchlorosilane (BSTFA+TMCS) (99:1) was added and the solution again incubated at 37° C. for 30 min with vortexing at 15 min intervals, then 5 μl of alkane mix (n-dodecane, n-pentadecane, n-octadecane, n-eicosane, n-pentacosane, n-heptacosane, n-dotriacontane at 0.029% w/v each) was added and mixed. Each derivatization mix was left at ambient temperature for 60 min before GC-MS analysis.

The GC-MS metabolite analysis was conducted on a Shimadzu TQ8050 gas chromatography tandem mass spectrometer fitted with a DB-5 capillary column (30 m×0.25 mm ID×1 μm film thickness). One μl was injected at 1:10 split mode onto the column with the inlet heated to 280° C. and helium as carrier gas. The oven temperature was set to 100° C., held for 4 min, then increased to 320° C. at 10° C./minute, and held for 11 min. The mass spectrometer interface was heated to 280° C., ion source at 200° C. Masses between 45 and 600 were measured in full-scan mode. For multiple reaction monitoring (MRM) mode, the Shimadzu MRM library containing 467 compounds with target and qualifier ions between particular retention time windows, set for each metabolite derivative, was used for detection with the same GC and MS parameters. Multiple reaction monitoring (MRM) parameters were developed for homocitric acid 4TMS and included in the MRM analysis protocol by scanning m/z=287, 243, 147, and 73 across collision energies 3-45 V. Based on the scan, the following two fragmentation patterns were used for detection at retention index 1931: target ions m/z=287>73 at 21 volts, and reference ions m/z=287/243 at 9 volts. To prevent contamination of the injection syringe from one sample to the next, the syringe was washed five times each with hexane followed by a 1:1 v/v solution of ethylacetate and acetone, followed by a rinse with pyridine to remove any residual homocitric acid 4TMS from the previous sample. Putative compounds identified in MRM mode were crosschecked against the chromatogram obtained in full-scan mode, where the mass spectrum at the particular retention time was searched against the NIST 17 library and Golm metabolome database (Hummel et al., 2007).

### Results for Homocitrate Production in Plant Cells

Homocitrate was readily detected and measured by this method in many of the samples. The control N. benthamiana leaf samples which had been infiltrated with the p19 construct alone without a NifV/HCS sequence showed low, background levels of homocitrate. The GC-MS/MS method was exceedingly sensitive, so it was not surprising that a low level of homocitrate was identified. The signal in the control plants was considered genuine since the method used two diagnostic ions and retention time against an authentic commercial standard. There was no background noise for those particular ions in quality control (QC) standard mixes or extraction buffer only.

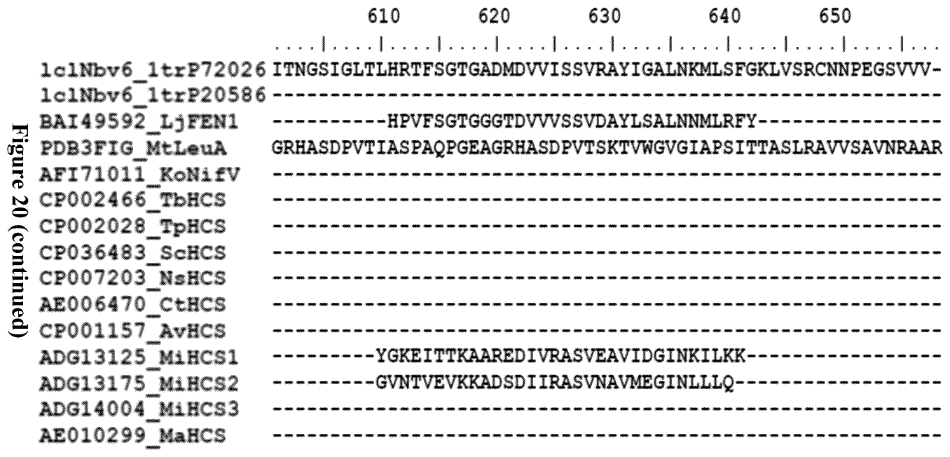

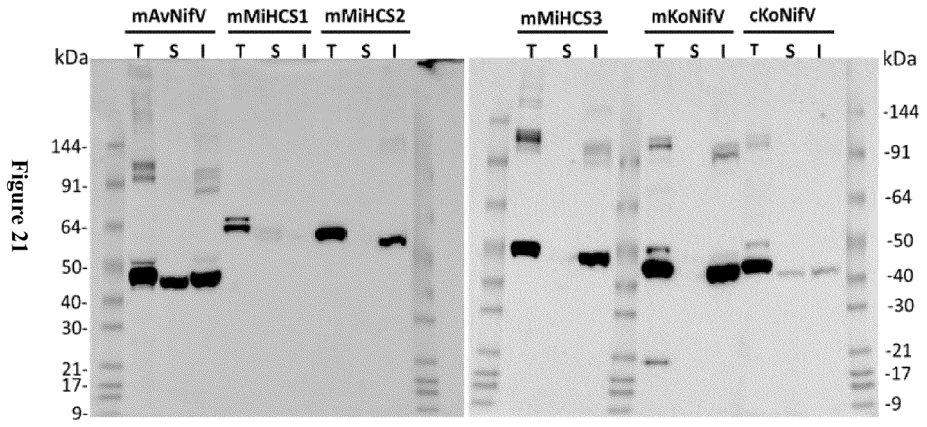

Non-infiltrated leaf samples and leaves inoculated with a gene encoding GFP also showed low, background levels of homocitrate. A baseline peak area was selected which had the highest amount of peak area of the three negative controls (GFP, p19, wild-type). For each sample infiltrated with a NifV/HCS gene, the baseline homocitrate target ion peak area was subtracted from the peak area for the test sample. The normalised peak areas were converted to a login scale and the data is presented in FIG. 23.

The data showed that the NifV/HCS polypeptides from K. oxytoca (KoNifV) and all three from Methanocaldococcus infernus (MiHCS1, MiHCS2 and MiHCS3) did not produce detectable homocitrate above the baseline level for both the mitochondrially-targeted and cytoplasmically-targeted polypeptides. These data were consistent with the observed insolubility of KoNifV and the MiHCSs for the mitochondrially-targeted polypeptides (FIGS. 20 and 21) but the lack of HCS activity for the cytoplasmically-targeted polypeptides was a mystery. The M. infernus polypeptides may have been inactive at the growth temperature of N. benthamiana. In contrast, both the mitochondrially-targeted and the cytoplasmically-targeted fusion polypeptides comprising seven of the other NifV/HCS sequences were clearly active in producing homocitrate in the leaf cells.

Several specific observations were particularly noteworthy. The S. cerevisiae HCS (ScHCS) polypeptides were the most active of the tested polypeptides in producing homocitrate, being 10- to 100-fold more active than the other polypeptides, regardless of the mitochondrial or cytoplasmic localisation. The 22 amino acid N-terminal extension (scar sequence) having the sequence ISTQVVRNRGGYPYDVPDYAGG (SEQ ID NO:166) including the HA epitope sequence on the N terminus of ScHCS was clearly tolerated for HCS function. A shorter, 12 amino acid scar sequence MYPYDVPDYAGG (SEQ ID NO:165) on the N terminus of ScHCS was also tolerated for function. Most surprisingly, the AvNifV fusion polypeptide of 406 amino acids from SN254 (encoding MTP::HA::AvNifV, processed to scar9-HA::AvNifV) produced 27-fold more homocitrate when it was targeted to plant mitochondria relative to the cytoplasmically-targeted polypeptide. This was also true, but less in extent, for the Chlorobaculumtepidum HCS (CtHCS) produced from SN253. The likely reason for these observations was that the AvNifV and CtHCS polypeptides were both somewhat oxygen sensitive, the mitochondrial location being more protected from oxygen and so yielding greater activity. At the same time, the definite homocitrate production when AvNifV and CtHCS were located in the cytoplasm suggested that those two polypeptides could tolerate oxygen to some extent. Oxygen sensitivity of AvNifV has been reported by Zheng et al. (1997). In similar fashion, the Thermincola potens, Thermoanaerobacter brockii and Methanosarcina acetivorans HCSs also produced homocitrate regardless of where they were located. Notably, these three HCSs were more active when located to the cytoplasm, indicating they were not as oxygen sensitive. As was observed with ScHCS, the 22 amino acid extension having the sequence ISTQVVRNRGGYPYDVPDYAGG (SEQ ID NO:166) on the N-terminus of AvNifV was tolerated for function.

ScHCS had the highest level of homocitrate production and therefore was considered by the inventors to be the most suitable NifV/HCS for use as part of a recombinant Nif pathway in plant mitochondria if high-level homocitrate production was desired. However, any of the other HCS sequences could be used for FeMoco synthesis since homocitrate forms part of a cofactor which is not used up in the nitrogenase reaction, so not much would be needed. The optimal level of NifV function can be determined empirically, as further described in the Examples below.

FeMoco synthesis and subsequent nitrogenase activity can be achieved in vitro by combining NifB, NifX, NifE, NifN, NifH, apoNifD-NifK, NafY, Mo, Fe, S, s-adenosylmethionine, ATP regenerating mixture (ATP, phosphocreatine, creatine phosphokinase) and R-homocitrate as reported by Curatti et al. (2007), which suggests that NifV does not need to interact physically with the other Nif components if the combination mixture is provided with sufficient homocitrate. It is thought that NifH, in particular, functions as an ATP-dependent Mo-homocitrate insertase to deliver Mo-homocitrate to the NifE-NifN complex for FeMoco assembly (Hu et al., 2013). The inventors considered that, if there was a possibly detrimental effect of producing high levels of homocitrate by ScHCS in plant mitochondria, then AvNifV would be more suited as part of a recombinant Nif pathway than ScHCS as the AvNifV enzyme was more likely to deliver the homocitrate it produced to NifH by physical association.

Measurement of α-Ketoglutarate and Pyruvate in the N. benthamiana Cells.

The GC-MS metabolite analysis also detected the derivatives α-ketoglutaric acid (αKG) 1MEOX 2TMS and pyruvic acid 1MEOX 1TMS, derivatives of αKG and pyruvic acid, respectively. αKG and acetyl-Coenzyme A (Ac-CoA), which is produced from oxidation of pyruvate by pyruvate dehydrogense, are the two substrates that NifV/HCS enzymes use to synthesise homocitrate. When ScHCS was expressed in N. benthamiana leaves, it reduced the level of αKG and pyruvate compared to when A. vinelandii NifV was targeted to the mitochondria, which were essentially the same levels as in the negative control leaves lacking NifV/HCS. Since αKG and pyruvate are key intermediates of the TCA cycle in the mitochondrial matrix, a decrease in their levels may have a detrimental effect on overall mitochondrial function, so over-expression of NifV/HCS to deleterious levels should be avoided. Therefore, it was concluded that AvNifV would be better suited for FeMoco, FeVco or FeFeco assembly than ScHCS if the homocitrate that was produced by AvNifV could be delivered to NifH via protein-protein interaction, rather than via diffusion that possibly requires a higher concentration of homocitrate.

### Example 16. Solubility of NifH Variants when Expressed in Plant Cells

### Introduction

The NifH polypeptide from Klebsiella oxytoca (KoNifH; SEQ ID NO:1) was found to be mostly insoluble, or in some experiments entirely insoluble, in plant mitochondria when expressed as a fusion polypeptide with an MTP sequence and a HA epitope sequence in a transient leaf expression system (Example 3). It was concluded that the NifH fusion polypeptide did not fold correctly in the N. benthamiana mitochondria or remained associated with the membranes, even though the MTP sequence had been cleaved correctly by MPP, and thus was unlikely to function properly as a NifH protein in that situation. In contrast, a corresponding NifH fusion polypeptide including the K. oxytoca NifH sequence but lacking the MTP sequence at the N-terminus, directed to the cytoplasm rather than the mitochondria, was soluble in the transient leaf expression system. The insolubility of the fusion polypeptide was therefore related to the mitochondrial localisation. Previously, Lopez-Torrejon et al. (2016) reported that NifH from Azotobacter vinelandii retained the electron transport function of NifH and was soluble in yeast mitochondria. However, it was also reported at a conference in Stockholm, Sweden, that NifH from A. vinelandii accumulated only at low levels when expressed in plant mitochondria in a transient leaf expression system, presumably due to low solubility (Xi Jiang at ENFC in Stockholm, 2018). It therefore appeared to the inventors that yeast cells and plant cells might differ with respect to the solubility and/or function of any one specific NifH polypeptide.

### Results

In an attempt to circumvent the problem of the apparent insolubility of the KoNifH fusion polypeptides that had been tested for high level expression in N. benthamiana, the inventors searched for homologues of NifH proteins from other organisms that might be soluble as fusion polypeptides in plant mitochondria, using the process described as follows.

NifH polypeptide sequences were extracted from the InterPro database using family IPR005977—Nitrogenase iron protein NifH—as the query, accessing the database on 23 Apr. 2018. 4183 NifH amino acid sequences were identified and extracted from the database. To select and test representative sequences, a protein network was established based on protein similarity, resulting in the clustering of NifH polypeptides based on sequence similarity. To do this, the amino acid sequences were aligned with MAFFT—Multiple alignment program for amino acid or nucleotide sequences—software, version 7 using the server mafft.cbrc.jp/alignment/server/large.html?aug31. The strategy G-large-INS-1 for less than 10,000 sequences, shorter than 5,000 sites, was used. The output was converted from .pir to .phy format using an online sequence converter (www.hiv.lanl.gov/content/sequence/FORMAT_CONVERSION/form.html). In order to calculate distance matrices and prepare the data in the input files for Cytoscape, the PHYLIP/protdist program was used to calculate the Kimura distance matrix for the NifH sequences. The output file was modified using Notepad++ to prepare an appropriate input format for the aMATReader in Cytoscape. The distance matrix was then modified in Excel to decrease file size and define subgroups: all values that were greater than 0.1 were removed, thereby creating subgroups, redundant sequences were removed, zero values were removed, and values were rounded to three decimal places.

This distance matrix was imported into Cytoscape using the aMATReader app as an undirected network, using delimiter: tab, deselect rows for import. At this stage, the network contained 3,114 nodes and 450,489 edges. The network was visualized using the prefuse force directed layout (unweighted). Additional information was extracted from the UniProt knowledgebase including entry name, status, protein names, gene names, organism, length and taxonomic lineage (PHYLUM) and imported into Cytoscape. Nodes were coloured by phylum and nodes representing sequences that were selected for biochemical analysis are displayed as larger nodes. Protein sequences that were longer than 700 amino acids were removed from the network—eleven protein sequences were removed as the length of these sequences (731-804 amino acid residues) was not in agreement with the length of a typical NifH protein at 260-300 amino acid residues. Nine of the 11 sequences were from Methanosarcina species, one was from Anaerovirgula multivorans and one from Treponema azotonutricium. These proteins were typically annotated as “NifEH”. The first part of each NifEH had a sequence that was similar to NifH including a P-loop, a [Fe₄S₄]-cluster binding site, and the second part of each sequence was related to NifE or NifD, respectively. In Methanosarcina, there is a gene coding for a NifD or a similar polypeptide located next to the gene for NifEH, but there is no NifK equivalent located in close proximity. These NifEH polypeptides might have a different function even though they were structurally related to the nitrogenase proteins. To the inventors' knowledge, such proteins have not been mentioned in the scientific literature and no experimental data is available.

The final network contained 3,103 nodes and 450,486 edges. The Cytoscape versions used for network generation and visualisation were 3.6.1 and 3.7.0. The InterPro database did not contain separate families for AnfH or VnfH proteins; therefore these were included in the NifH group. The contributing signatures from the InterPro member databases, namely CDD, TIGRFAMs and HAMAP, did not discriminate between NifH, AnfH and VnfH. The AnfH and VnfH sequences were therefore also included in the alignment. A subset of AnfH sequences was identified from the NifH sequences.

### Sequence Selection

A representative of each clustered group that containing more than 13 sequences was selected for biochemical analysis, for comparison to K. oxytoca NifH. This included NifH sequences from thermophilic nitrogen-fixing organisms to be tested for solubility and functional analysis (Table 17). The column for temperature in Table 17 indicated the optimal growth temperature for some of the organisms. The extent of sequence identity of each of the selected NifH sequences to SEQ ID NO:1 is shown in Table 18. The amino acid sequences of the fusion polypeptides comprising the selected NifH polypeptides other than KoNifH, fused at the C-terminus of the MTP-CoxIV-TwinStrep sequence, are provided in SEQ ID NOs:168 to 181.

TABLE 17
NifH sequences from nitrogen-fixing and related organisms, tested for solubility
when expressed as fusion polypeptides in N. benthamiana leaves.

| | Temperature | SEQ | Genetic | |
|---|---|---|---|---|
| Source Organism | [° C.] | ID NO | Construct | Reference |
| K. oxytoca | | 1 | SN41 | |
| Azospirillum brasilense | | 168 | SN178 | |
| Mastigocladus | 45-50 | 169 | SN179 | Miyamoto et al. |
| laminosus | | | | (1979); |
| (Fischerella) | | | | Khumanthem et |
| | | | | al. (2007). |
| Frankia casurinae | | 170 | SN180 | |
| Marichromatium | max 44 | 171 | SN181 | Serrano et al. |
| gracile biotype | | | | (2009). |
| thermosufidiphilum | | | | |
| Methanocaldococcus | | 172 | SN182 | Mehta and |
| infernus | | | | Baross, (2006). |
| Heliobacterium | 50 (max 56) | 173 | SN183 | Kimble et al. |
| modesticaldum | | | | (1995). |
| Chlorobaculum | 48-50 | 174 | SN184 | Wahlund and |
| tepidum (Chlorobium | | | | Madigan, |
| tepidum) | | | | (1993). |
| Geobacter sp. M21 | | 175 | SN185 | |
| Bradyrhizobium. | | 176 | SN186 | |
| diazoefficans | | | | |
| Methanobacterium | 40-70 (opt 65) | 177 | SN187 | Smith et al. |
| thermoautotrophicum | | | | (1997). |
| Methanosarcina | | 178 | SN188 | |
| Desulfotomaculum | 36 | 179 | SN189 | |
| acetoxidans | | | | |
| Carboxydothermus | carboxydotrophic | 180 | SN190 | Yoneda et al. |
| pertinax | 50-70 (opt 65) | | | (2012). |
| Nostoc calcicola | | 181 | SN191 | |

The Carboxydothermus pertinax strain was probably not capable of nitrogen fixation since the NifD protein coding region in that organism had an internal stop codon. Therefore, the NifH sequence might also not have been functional.

TABLE 18
Amino acid sequence identity to NifH from K. oxytoca (SEQ ID NO: 1).

| | % sequence |
|---|---|
| NifH from: source organism | identity |
| Azospirillum brasilense | 72.4 |
| Mastigocladus laminosus (Fischerella sp.) | 72.1 |
| Frankia casuarinae (strain DSM 45818/CECT 9043/CcI3) | 75.1 |
| Marichromatium gracile (Chromatium gracile) | 85.0 |
| Methanocaldococcus infernus (strain DSM 11812/JCM 15783/ME) | 56.8 |
| Heliobacterium modesticaldum (strain ATCC 51547/Ice1) | 74.9 |
| Chlorobaculum tepidum (strain ATCC 49652/DSM 12025/NBRC | 65.8 |
| 103806/TLS) (Chlorobium tepidum) | |
| Geobacter sp. (strain M21) | 79.9 |
| Bradyrhizobium diazoefficiens (strain JCM 10833/IAM 13628/NBRC | 74.6 |
| 14792/USDA 110) | |
| Methanothermobacter thermautotrophicus(strain ATCC 29096/DSM | 59.5 |
| 1053/JCM 10044/NBRC 100330/Delta H) (Methanobacterium | |
| thermoautotrophicum) | |
| Methanosarcina barkeri | 61.4 |
| Desulfotomaculum acetoxidans (strain ATCC 49208/DSM 771/VKM B- | 64.8 |
| 1644) | |
| Carboxydothermus pertinax | 61.0 |
| Nostoc calcicola FACHB-389 | 56.3 |

### Solubility Testing for NifH Proteins in Transient Leaf Expression System

The solubility of the different NifH polypeptides when expressed as MTP-CoxIV::TwinStrep::NifH fusions for plant mitochondrial localisation was assessed by the Western blot method using a strep-antibody. The TwinStrep sequence was placed between the MTP and NifH sequences. This epitope was used to allow for the subsequent purification of the NifH fusion polypeptides, if desired. Protein extracts were made from the infiltrated leaf tissues and fractionated under aerobic conditions for soluble and insoluble fractions. The solubility of each NifH fusion polypeptide was assessed when it was co-expressed with K. oxytoca NifM expressed from the genetic construct SN44 (encoding MTP-FAγ51::NifM::HA), to see if the co-expression with NifM might increase the solubility. NifM is thought to be involved in maturation of NifH in K. oxytoca. It was not known if the NifH polypeptides from the other species tested required a NifM-like protein for full activity. Most of those organisms other than the proteobacteria do not contain a NifM homologue in their genome, but other non-homologous proteins might perform a similar function instead of NifM.

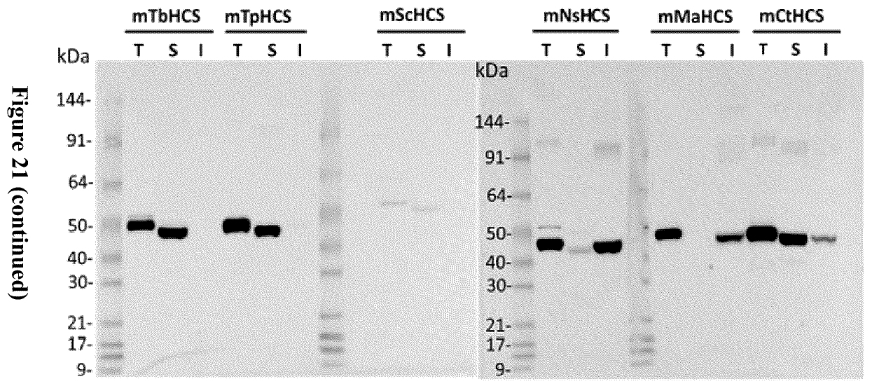

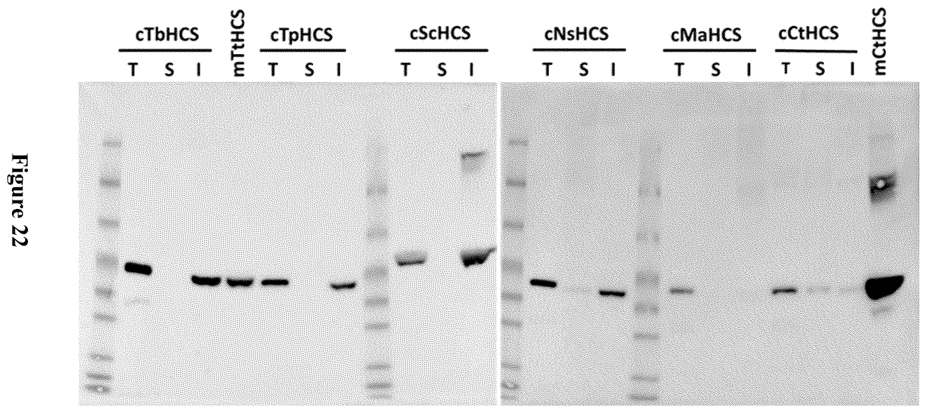

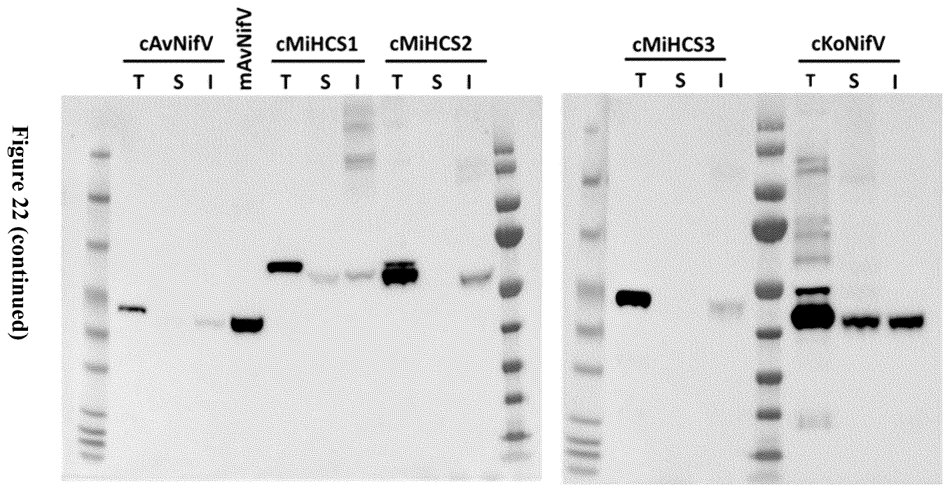

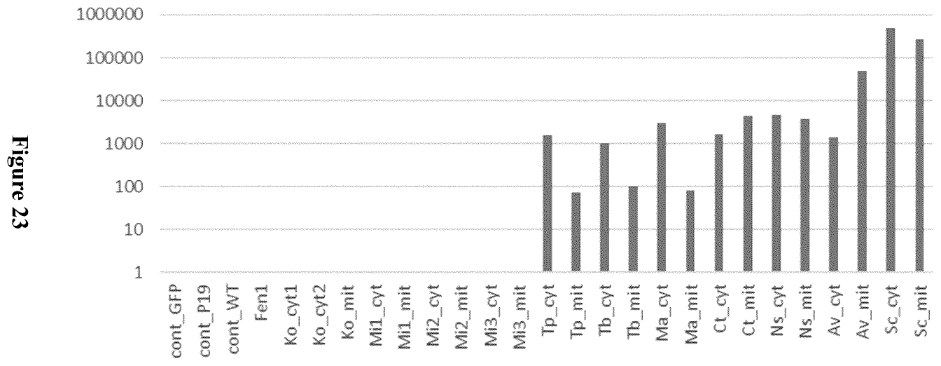

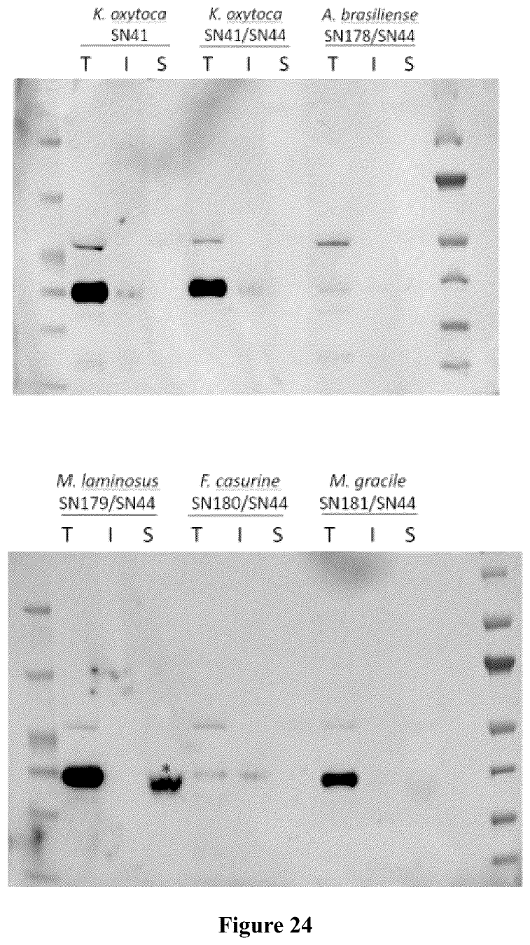

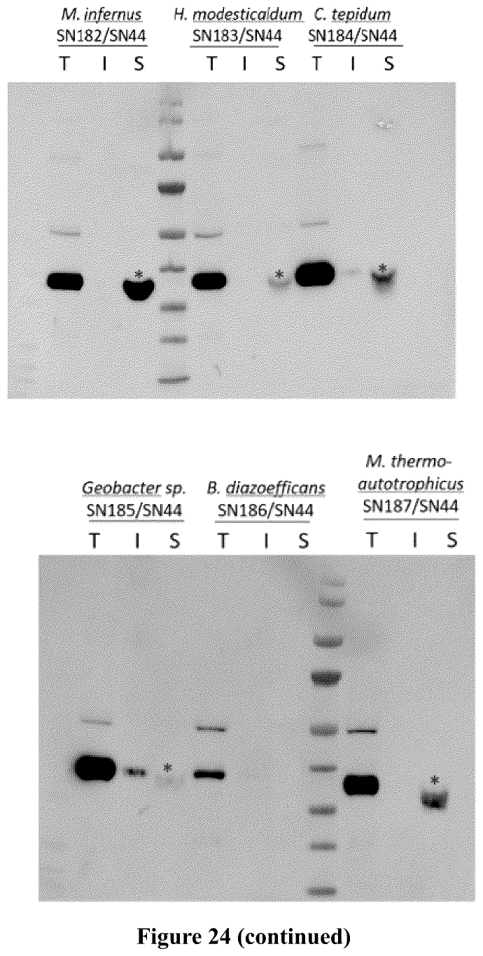

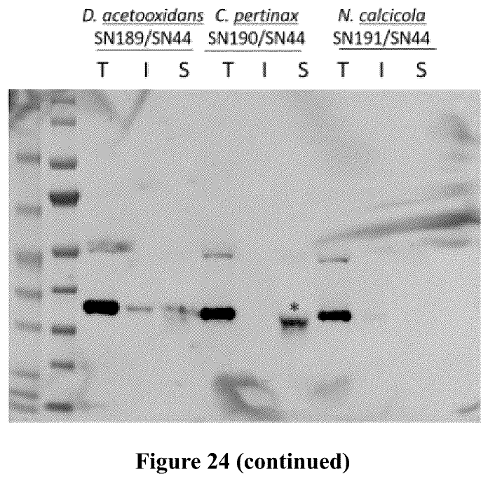

The Western blot analysis (FIG. 24) showed that soluble, or at least partly soluble, NifH protein was detected for the fusion polypeptides including the NifH sequences from M. laminosus, M. infernus, H. modesticaldum, C. tepdium, Geobacter sp. M21 and M. thermoautotrophicus. There was little or no NifH fusion polypeptide detected in the soluble fraction for those including NifH from K. oxytoca, A. brasilense, F. casurinae, M. gracile and B. diazoefficans. It was concluded that most of the NifH polypeptides that were at least partially soluble in plant cell mitochondria were derived from thermophilic bacteria, possibly because such polypeptides were inherently more stable than those from mesophilic bacteria and so would more readily able to fold into, and maintain, their native conformations.

It was also observed that there was no significant increase in the solubility when NifH fusion polypeptide was co-expressed with K. oxytoca NifM, including for the K. oxytoca NifH (FIG. 24). As mentioned above, it was not known whether the NifH proteins in most of the bacterial species tested required a NifM-like activity for maturation and production of a fully functional NifH.

Purification of M. Infernus NifH and M. Laminosus NifH from N. benthamiana Leaves

The Twin Strep::NifH fusion polypeptides for NifH from M. infernus and M laminosus were successfully purified from the infiltrated N. benthamiana leaf samples after extraction under non-denaturing conditions and then using a StrepTactinXT column. This confirmed that the MPP-processed NifH fusion polypeptides from these two species were indeed soluble in the mitochondria of the leaf cells. The purified proteins are used for biochemical analysis, such as analysis for the presence of FeS clusters and the ability of the NifH polypeptides to donate electrons to purified NifD-NifK isolated from A. vinelandii.

### Testing of Variant NifH Polypeptides in a Bacterial Nitrogenase System

The NifH candidates that were found to be soluble when targeted to leaf mitochondria were tested for NifH function in E. coli using the MIT2.1 system. A XhoI site was introduced into the 3′ end of the NifH protein coding region in MIT2.1 by site directed mutagenesis. This newly introduced XhoI restriction site along with an existing XhoI site upstream of NifH was used to separately replace the wild-type K. oxytoca NifH sequence in MIT2.1 with seven selected NifH variant sequences that were synthesised with XhoI restriction sites flanking each open reading frame: M laminosus NifH (MiNifH; Genbank Accession No. Q47917), M. infernus NifH (MiNifH; Genbank Accession No. WP_013099459), H. modesticaldum NifH (HmNifH; Genbank Accession No. WP_012282218), C. tepidum NifH (CtNifH; Genbank Accession No. WP_010933198), Geobacter sp. NifH (GspNifH; Genbank Accession No. WP_015837436), M thermautotrophicus NifH (MtNifH; Genbank Accession No. AAB86034), and Carboxydothermus pertinax NifH (CpNifH; Genbank Accession No. WP_075859892). Replacement of the NifH variants were done with pHJ-TOPO containing K. oxytoca NifHDKYENJ, then pB-ori containing the latter half of MIT2.1 of K. oxytoca NifBQFUSVWZMwas ligated to the modified pHJ-TOPO after both plasmids were digested with SbfI.

The resulting modified MIT2.1 plasmids containing the NifH variants were used to transform E. coli strain JM109 and the transformants tested in the acetylene reduction assay. Compared to JM109 with the original MIT2.1 as positive control in the ARA, none of the JM109 strains carrying the modified MIT2.1 plasmids with the NifH variants reduced acetylene, instead showing the same background level of ethylene production as JM109 carrying the negative control plasmid pB-ori. Based on this result, the inventors concluded that the NifH variants would not function with the NifD-NifK proteins from K. oxytoca but would function with their corresponding NifDK heterotetramer, for example C. tepidum NifH with C. tepidum NifD-NifK. The compatibility of each NifH with NifD-NifK can therefore be determined empirically.

### Example 17. Expression of NifH and NifM in Stably Transformed Plants

### Introduction

A functional NifH protein, also known as the Fe protein, is essential for nitrogenase activity. It has several known functions for nitrogenase activity: it is required for donation of electrons to the nitrogenase enzyme, for maturation of the metalloclusters including the P-cluster and is involved in the synthesis of the cofactors FeMoco, FeVco and FeFeco for the Mo-nitrogenase, V-nitrogenase and Fe-nitrogenase, respectively. Previously Rubio and colleagues have co-expressed Azotobacter vinelandii NifH and NifM within yeast with targeting to the mitochondria. Purified NifH protein from the yeast cells was capable of electron donation in vitro to a holo-NifD-NifK complex (Lopez-Torrejon 2016) but the other functions of the NifH polypeptide were not tested in that in vitro system, which was not designed for that. The introduction of a fully functional NifH within an organelle such as plant mitochondria will be essential for engineering nitrogenase into plants.

The inventors have previously shown using a transient system in N. benthamiana leaves that Klebsiella oxytoca NifH (KoNifH) could be targeted to the plant mitochondria by translationally fusing an MTP sequence to the N-terminus of the KoNifH polypeptide (Allen et al 2017). The fusion polypeptide was well expressed and processed by cleavage within the MTP sequence, demonstrating mitochondrial localisation of the expressed fusion polypeptide. When translationally fused at the N-terminus of KoNifH, two different mitochondrial targeting peptides MTP-FAγ77 and MTP-FAγ51 were efficiently and specifically cleaved at the expected site within the MTP. The abundance of the processed NifH fusion polypeptide was relatively high compared to other mitochondrially-targeted Nif proteins. Furthermore, the experiments described in Example 4 herein demonstrated that the 9 amino acid “scar sequence” from the C-terminus of the MTP sequence left after cleavage by mitochondrial matrix protease (MPP) with an additional Gly-Gly linker, 11 amino acids in total, at the N-terminus of KoNifH did not reduce acetylene reduction activity when assayed in a bacterial complementation format.

However, in the case of the MTP-FAγ51::KoNifH::HA fusion polypeptide (SEQ ID NO:25) encoded by the vectors SN18 and SN27, the MPP-cleaved polypeptide scar9::KoNifH::HA was found almost exclusively within the insoluble protein fraction (Examples 2 and 3). To assess whether the insolubility of NifH might be due to the targeting peptide, another genetic construct (SN42) was made and tested using a different MTP sequence, encoding a MTP-CoxIV::TwinStrep::KoNifH::HA (SEQ ID NO:128). Although a correctly processed form derived from MTP-CoxIV::TwinStrep::KoNifH::HA was readily detected in the transient leaf assays after cleavage at the expected site within the MTP, this scar32::KoNifH::HA product was also found predominantly in the insoluble protein fraction.

As insoluble proteins are likely to be misfolded or remain bound to membranes and therefore non-functional, the inventors sought various alternative methods to improve the solubility of NifH, described as follows. The functional consequences of a 32 amino acid N-terminal extension to KoNifH was also tested, corresponding to the polypeptide produced after cleavage of the MTP-CoxIV::TwinStrep::KoNifH::HA fusion polypeptide by the MPP in plant mitochondria.

Genetic and biochemical studies with Azotobacter vinelandii and Klebsiella oxytoca showed that NifM was required for the production of a functional and mature NifH protein in those nitrogen fixing bacteria. As shown in Examples 2-4 herein, a mitochondrially targeted version of K. oxytoca NifM, MTP-FAγ51::KoNifM::HA (SEQ ID NO:123) was expressed, cleaved accurately and efficiently within the plant mitochondria, and detected in the soluble fraction. However, the 9 amino acid scar sequence at the N-terminus of KoNifM reduced acetylene reduction activity to only 10-20% of the wild-type level in the E. coli MIT2.1 system (Table 4). Proteomic analysis of the bacterial strains producing the processed scar9::NifM::HA polypeptide showed that this N-terminal addition to NifM resulted in about a 50-fold increase in the accumulation of the modified NifM polypeptide relative to the wild-type NifM. As it is known that nitrogenase activity is sensitive to changes in expression levels of the various Nif proteins (Temme et al., 2012), it is plausible that the excess of scar9::NifM in that bacterial assay format reduced the nitrogenase function to 10-20% of the wild-type level.

Co-Expression of NifH and NifM in N. benthamiana Leaves

In order to test whether the solubility of NifH within plant mitochondria might be increased by co-expression with a NifM fusion polypeptide that was also targeted to that organelle, a mixture of Agrobacterial strains each containing a different vector was infiltrated into N. benthamiana leaves as described in Example 1. A first strain that comprised either a vector encoding MTP-FAγ51::KoNifH::HA (SN18) or the vector encoding the MTP-CoxIV::TwinStrep::KoNifH::HA fusion polypeptide (SN42) and a second strain that comprised SN30 encoding the MTP-FAγ51::KoNifM::HA fusion polypeptide were mixed and infiltrated. Five days after infiltration, total, soluble and insoluble protein fractions were prepared from the leaf tissues and subjected to Western blot analysis. There was no consistent increase in the solubility of the NifH polypeptide in these combinations relative to infiltrations with a single vector.

As a further attempt, another vector was constructed which had two genes on the same T-DNA, one gene encoding the MTP-CoxIV::TwinStrep::KoNifH::HA fusion polypeptide (SEQ ID NO:128) and the other encoding a MTP-FAγ51::HA::KoNifM polypeptide (SEQ ID NO:167). The first gene had a TwinStrep epitope between the MTP and NifH sequences and a HA epitope at the C-terminus. The second gene had a HA epitope between the MTP and NifM sequences. The genetic construct with the two genes was designated SL6. It was constructed using the modular DNA assembly system referred to as the GoldenGate method, as described above. The gene encoding the KoNifH fusion polypeptide was under the control of the enhanced 35S promoter, while the gene encoding the KoNifM polypeptide was the SCSV S₄ promoter (Accession No. AY181084).

Agrobacterium cultures transformed with SL6 were infiltrated into N. benthamiana leaves. Samples were harvested five days after infiltration and total, soluble and insoluble protein fractions were prepared. Western blot analysis of the protein extracts indicated that the co-expression of both the NifH and NifM fusion polypeptides from the same vector did not consistently increase the solubility of NifH, although at least one experiment appeared to show an increase in the amount of soluble NifH polypeptide.

It was then decided to use SL6 to transform N. tabacum (tobacco) and N. benthamiana to produce stably-transformed plants with the T-DNA integrated into the plant nuclear genome.

### Plant Transformation Protocol

To transform N. benthamiana plants, plants were grown aseptically in tissue culture as a source of plant material for transformation. The source plants were established from surface sterilised seeds. To do this, seeds were rinsed with 70% ethanol, then surface sterilised with 5% sodium hypochlorite for 10 mins with agitation, followed by rinsing in several changes of water. The seeds were then germinated on plates containing MSO medium at 4.43 g/L (M519, PhytoTechnology Laboratories) containing 3% sucrose and 0.8% agar, at pH 5.8. Plants were grown in a growth room at 26° C. using a 16/8 hour photoperiod. After approximately 2 weeks, developing seedlings were transferred and thinned out to 4 seedlings per deep tissue culture plate and cultured on the same medium and growth conditions. About 2 weeks later, single well-established plants were cultured in tissue culture pots. Leaves from 6-week-old N. benthamiana plants were used for Agrobacterium-mediated transformation.

Cultures of A. tumefaciens strain AGL1 containing the genetic constructs in binary vectors such as SL6 were grown at 28° C. in MG/L medium with antibiotics to maintain selection for the genetic constructs. Cultures having an optical density of between 0.25-0.5 at 600 nm were used to inoculate the N. benthamiana tissues, as follows. Upper leaves from the tissue-culture grown plants were excised and floated on MG/L medium to maintain turgidity until used, and cut into pieces about 1 cm 2, including leaf midribs. The Agrobacterium culture containing the genetic construct was added to the leaf pieces, ensuring the explants were completely wet, and left for 20-30 mins with occasional shaking to allow the bacteria to bind to the plant cells along the cut edges. The inoculated explants were then lightly blotted on sterile filter paper to remove excess Agrobacteria and transferred adaxial side down to Co-cultivation Medium MS9 without antibiotics. MS9 contained MSO medium with 3% sucrose, 0.8% agar at pH 5.8, sterilised by autoclaving, and the plant hormones IBA at 1 mg/L and IAA at 0.5 mg/L added after the autoclaving and cooling of the MSO-agar medium to 55° C. The inoculated explants were co-cultivated at 26° C. for 48 h in the dark. Following the co-cultivation period, the explants were transferred to shoot regeneration medium (MS9 and the plant hormones IBA at 1 mg/L and IAA at 0.5 mg/L plus kanamycin at 100 mg/L and Timentin at 150 mg/L), adaxial side up, plating about 10 explants per plate. These were incubated at 26° C. under lighting with a 16/8-hour photoperiod. The explants were transferred to fresh shoot regeneration medium every 2-3 weeks until shoot development occurred. After 6-8 weeks, shoots that had developed to sufficient size were transferred to root initiation medium (⅓ MSO+100 mg/L kanamycin+150 mg/L Timentin+1 mg/L IBA). Once individual plants had developed strong roots, small leaf samples were harvested for DNA extraction and testing by PCR for the presence of the selectable marker gene and the desired transgenes. Confirmed transgenic plants were then planted in soil and grown in a glasshouse, allowing the plants to acclimatise gradually.

Nicotiana tabacum plants of cultivar Wisconsin 38 (Wi38) were transformed by standard methods (Borsch et al., 1985).

Twelve independently transformed plants were generated with SL6 in N. benthamiana, designated SL6-1 to 12, and another twelve generated in N. tabacum, SL6-13 to 24. These initial transgenic plants were referred to as the TO generation. The presence of the T-DNA in each of the plants was confirmed using PCR on DNA prepared from leaf samples from the plants, confirming that all of the plants were transgenic. These independently transformed plants were grown to maturity and T1 seeds harvested after self-fertilisation of each plant. To test segregation of the transgene in one line, 60 T1 seeds from the plant designated SL6-13 were sown into soil and grown for four weeks under standard glasshouse conditions. The presence of transgenes was assessed using PCR. Twenty plants lacked the transgene (null segregants) and 40 plants were PCR positive, indicating a low copy number transgenic event, probably having one T-DNA insertion in plant SL6-13. Several null segregants were identified and maintained as negative controls.

The production of the NifH and NifM fusion polypeptides in the transgenic plants was assessed by extraction of total protein and detection using either anti-Strep antibody or anti-HA antibody in Western blots. The level of NifH fusion polypeptide in the stably transformed tobacco plants was much lower than the levels observed previously in transient expression in N. benthamiana leaves. Surprisingly and unexpectedly in view of the earlier results from those experiments, the tobacco plants including a plant designated SL6-13 produced detectable levels of correctly processed NifH that was found exclusively within the soluble fraction. Likewise, N. benthamiana plants stably transformed with SL6 produced significantly less NifH polypeptide, but the polypeptide had been processed efficiently and was also found in the soluble fraction.

### Analysis of Progeny Transgenic Plants

Leaves of various ages were harvested from progeny plants to see if there was any effect of leaf age on the accumulation, processing and solubility of the NifH and NifM fusion polypeptides. Samples were taken from two N. tabacum plants that were progeny from SL6-13, taking a young leaf, a “middle aged” leaf and an older leaf from each plant. The NifH fusion polypeptide was detected by Western blotting using anti-Strep antibody and the NifM polypeptide with anti-HA antibody in each of the leaves. The level of accumulation of the NifH fusion polypeptide increased with the age of the leaves.

Purification of the NifH Fusion Polypeptide from the Stably Transformed Plants

Given that the TwinStrep tagged NifH polypeptide was soluble and that sufficient plant material was available, this polypeptide was purified using the StreptactinXT affinity media. Approximately 90 g of SL6-13 plant leaf material was extracted by homogenising the material in a non-denaturing buffer, centrifugation to remove cellular debris, filtration through 0.22 μm filters and passage through a StreptactinXT column. After elution from the column using biotin, fractions containing the NifH polypeptide were collected and concentrated. Samples were analysed by proteomics and subjected to Western blot analysis with anti-Strep antibody to detect the NifH polypeptide and anti-HA antibody to detect both the NifH and NifM polypeptides (FIG. 25). The purified protein was subjected to N-terminal analysis to determine the amino acid sequence at the N-terminus. These analyses confirmed that the CoxIV MTP was cleaved at the predicted MPP cleavage site. Purification of NifH by binding to the StreptactinXT column also supported the conclusion that the TwinStrep::KoNifH extracted from the stably transformed plants was soluble. Overall, these results indicated that the scar32::TwinStrep::KoNifH::HA protein isolated from stably transformed N. tabacum plants had been correctly processed within the mitochondria and was fully soluble, fulfilling two major requirements for NifH function within plants.

Co-Expression of NifS and NifU with NifH and NifM in the Transformed N. benthamiana Plants

Genetic constructs encoding NifS (SN31) and NifU (SN32) fusion polypeptides were infiltrated into the N benthamiana plants transformed with SL6, to see whether co-expression of the NifS and NifU fusion polypeptides might increase the level of NifH polypeptide accumulation.

### Example 18. Expression of Anf Polypeptides in Plant Cells

### Introduction

The iron-only nitrogenase system is found in some diazotrophic bacteria, for example in A. vinelandii which has three nitrogenase systems based on molybdenum (Mo), vanadium (V) and iron-only (Fe), using the cofactors FeMoco, FeVco and FeFeco, respectively (Davis et al., 1996; Robson et al., 1986). Both the molybdenum (Mo-nitrogenase) and the vanadium nitrogenase (V-nitrogenase) enzymes that actually catalyse the reduction of dinitrogen have known crystal structures. While the iron-only nitrogenase (Fe-nitrogenase) crystal structure is yet to be established, it is thought to have a similar structure to that of the vanadium nitrogenase (Sippel & Einsle, 2017). All organisms recorded to date that contain one or both of the V- or Fe-nitrogenases also contain the Mo-nitrogenase. Generally, the V- and Fe-nitrogenases are repressed by the expression of the Mo-nitrogenase and are expressed only when the availability of Mo becomes limiting. To distinguish between the molybdenum-type nitrogenase and alternative nitrogenase, the Isotopic Acetylene Reduction Assay (ISARA) can be used which measures ¹³C isotopes in the acetylene reduction assay (Zhang et al., 2016).

The Fe-nitrogenase is the least studied of the three systems. It has the lowest nitrogenase catalytic activity of the three systems but its biogenesis appears to be simpler, requiring less proteins for nitrogenase activity. There are 6 known Fe-nitrogenase proteins from the well-studied organism Azotobacter vinelandii, namely AnfD, AnfK, AnfH, AnfG, AnfO and AnfR, which are distinct for Fe-nitrogenase. Of these 6 proteins, the first 4 are known to be required and to contribute to the activity of the nitrogenase enzyme. Each nitrogenase system requires catalytic proteins designated as Nif (or Vnf or Anf) D, K and H, and the Fe-nitrogenase uses the AnfD, AnfK and AnfH proteins. The V- and Fe-nitrogenases also require the additional structural protein designated VnfG or

AnfG, respectively, which is not required by the Mo-nitrogenase. The anfO and anfR genes are located downstream from the other structural anf genes but their function is not known and they have been shown to not affect the activity of the Fe-nitrogenase when expressed in an E. coli system (Yang et al., 2014). The remaining minimal accessory genes required for activity of the Fe-nitrogenase are common to the Mo-nitrogenase pathway, namely NifS, NifU, NifB, NifV, NifJ and NifF (Yang et al., 2014). The iron-only nitrogenase therefore has a minimal set of 4 Anf and 6 accessory Nif polypeptides required for heterologous function within E. coli (Yang et al., 2014).

In the Fe-nitrogenase system, the dinitrogenase enzyme which is the site of dinitrogen reduction is a heterohexamer made up of two AnfD polypeptides as the a unit, two AnfK polypeptides as the 13 unit and two AnfG polypeptides as the 6 unit, so in a α₂β₂δ₂ conformation. The dinitrogenase reductase enzyme, the obligate electron donor to the dinitrogenase enzyme, is a homodimer with 2 identical AnfH polypeptides. Dinitrogenase reductase is also known as the Fe protein and contains a single [Fe₄S₄] cluster at the interface of its subunits (Buren, Young, et al., 2017). The AnfH protein is also predicted to have two other functions including being required for the maturation of the dinitrogenase enzyme in analogous fashion to the NifH and VnfH gene products in the Mo- and V-nitrogenases.

As for the Mo- and V-nitrogenases, the engineering of plants to express the Fe-nitrogenase is considered to be exceedingly difficult. All of the key nitrogenase enzymes require a specific biochemical environment, being extremely oxygen sensitive, and require large quantities of ATP, a source of reductant, and elements such as Fe, Mo, V and S must be available in sufficient quantities in the correct cellular compartment. In particular, the Anf enzymes quickly become irreversibly inactivated when exposed to oxygen. As mentioned above, a minimal set of 4 Anf and 6 accessory Nif polypeptides would need to be introduced into a plant, which is very difficult to do from a technical viewpoint.

Experiments were therefore carried out seeking to express Anf genes in plant cells, aiming for mitochondrial localisation of the Anf gene products, as described below. Since the 4 key Anf proteins are the AnfD, AnfK, AnfH and AnfG proteins, the inventors first tested 4 genetic constructs each expressing an individual Anf gene, and then combined the 4 genes into one T-DNA in one vector.

### Single Gene Constructs to Express Anf Fusion Polypeptides in Plant Cells

A first series of genetic constructs was designed and made to separately express the AnfD, AnfK, AnfH and AnfG polypeptides in plant cells such as N. benthamiana leaf cells. Each synthetic gene was under the control of the strong 35S promoter and a CaMV 3′ polyadenylation region/transcriptional terminator which flanked the protein coding region. The Anf sequences from A. vinelandii were used to design the encoded amino acid sequences, and the nucleotide sequences were codon-optimised for expression in plant cells. For mitochondrial localisation, the constructs encoded fusion polypeptides having the MTP-FAγ51 fused to the N-terminus and either a HA- or a TwinStrep-epitope for detection of the polypeptides by Western blotting with anti-HA or anti-Strep antibodies, respectively. The HA epitope was fused translationally either C-terminally or, in most cases, between the MTP and Anf sequences, whereas the TwinStrep epitope was fused to the C-terminus of the Anf sequences. For each genetic construct that encoded a mitochondrially-targeted fusion polypeptide, two corresponding control constructs were also made. The first encoded a polypeptide which lacked the MTP sequence and therefore expressed a smaller, cytoplasmically-targeted polypeptide that provided a molecular weight comparator on the Western blots for an MPP-processed polypeptide from the MTP-Anf polypeptides (processed Anf), with the qualification that the MPP-processed polypeptide in each case included a “scar sequence” of about 9 amino acids and so the sizes were not identical. The second control construct in each case encoded a fusion polypeptide which had 13 amino acids in the MTP sequence substituted with alanines (Allen et al., 2017), designed to prevent processing by MPP. These second control polypeptides therefore provided a molecular weight comparator for the unprocessed polypeptide from the corresponding MTP-Anf construct. The alanine-mutated MTP sequence was designated herein as mFAγ51. When protein extracts from the infiltrated plant tissues were analysed, the sample from each MTP-Anf construct and its two corresponding control constructs were loaded onto adjacent lanes for gel electrophoresis, so allowing for the best detection of processing of the MTP-Anf polypeptide. Subsequently, the expected site of cleavage within the MTP motif was confirmed by mass spectrometry.

Where retention of AnfK function was desired for fusion polypeptides having the AnfK sequence, C-terminal extensions relative to the wild-type polypeptide were avoided. The desirability of using the wild-type C-terminal sequence for AnfK was analogous to the use of the wild-type C-terminus for NifK from K. oxytoca (WO2018/141030), since C-terminal extensions abolished function (Yang et al., 2017).

The single gene constructs are listed in Table 19, which also lists the predicted molecular weight (kDa) of each polypeptide before and after processing in the mitochondria by MPP. Table 19 also lists the SEQ ID NOs for the unprocessed fusion polypeptides. The genetic constructs were made using the GoldenGate assembly methods in analogous fashion to the constructs described in the earlier Examples.

As per Table 19, the control constructs for the AnfD constructs (SN81 and SN161) were SN82 which produced a polypeptide corresponding in approximate size to the processed form and SN158 which produced a polypeptide having the size of the unprocessed form. Protein extracts from these constructs were therefore run in adjacent lanes in the gel electrophoresis step of the Western blot analysis. For the AnfK construct SN129, the controls were SN152 and SN155. For the AnfH construct SN130, the controls were SN153 and SN156. For the AnfG construct SN131, the controls were SN154 and SN157.

Aside from varying the position of the HA epitope at either the C-terminus or toward the N-terminus, another variation that was made in one construct (SN195) was to use a CoxIV MTP sequence (Buren et al., 2017) rather than the MTP-FAγ51 sequence.

TABLE 19
Single gene constructs for expression of Anf fusion
polypeptides in plant cells. (NA: not applicable)

| | | | | SEQ ID |
|---|---|---|---|---|
| | | Predicted molecular | | NO for |
| | | weight (kDa) | | Unpro- |
| Plasmid | Expressed polypeptide | Unprocessed | Processed | cessed |
| SN81 | MTP-FAγ51::AnfD::HA | 65.4 | 60.7 | 182 |
| SN82 | HA::AnfD | 59.7 | NA | 183 |
| SN129 | MTP-FAγ51::HA::AnfK | 58.1 | 53.5 | 184 |
| SN130 | MTP-FAγ51::HA::AnfH | 36.9 | 32.4 | 185 |
| SN131 | MTP-FAγ51::HA::AnfG | 22.2 | 17.7 | 186 |
| SN152 | HA::AnfK | 52.5 | NA | 187 |
| SN153 | HA::AnfH | 31.3 | NA | 188 |
| SN154 | HA::AnfG | 16.6 | NA | 189 |
| SN155 | mFAγ51::HA::AnfK | 57.4 | NA | 190 |
| SN156 | mFAγ51::HA::AnfH | 36.3 | NA | 191 |
| SN157 | mFAγ51::HA::AnfG | 21.6 | NA | 192 |
| SN158 | mFAγ51::HA::AnfD | 64.6 | NA | 193 |
| SN161 | MTP-FAγ51::HA::AnfD | 65.3 | 60.7 | 194 |
| SN177 | MTP-FAγ51::AnfD::Twin | 69.2 | 64.5 | 195 |
| | Strep | | | |
| SN195 | MTP-CoxIV::Twin | 57.7 | 54.7 | 196 |
| | Strep::AnfK | | | |

Expression of Anf Fusion Polypeptides in N. benthamiana Leaf Cells

Each of the constructs was separately introduced into N. benthamiana plants by Agrobacterium-mediated methods as described in Example 1. Leaf samples were harvested 4-5 days post-infiltration and protein extracts prepared and analysed by SDS-polyacrylamide gel electrophoresis (PAGE) and Western blot as described in the earlier Examples. The expressed polypeptides were thereby tested for mitochondrial matrix import by virtue of the processing of the MTP leader sequences by MPP. In further experiments, protein extracts were fractionated into soluble and insoluble fractions using the method described in Example 1.

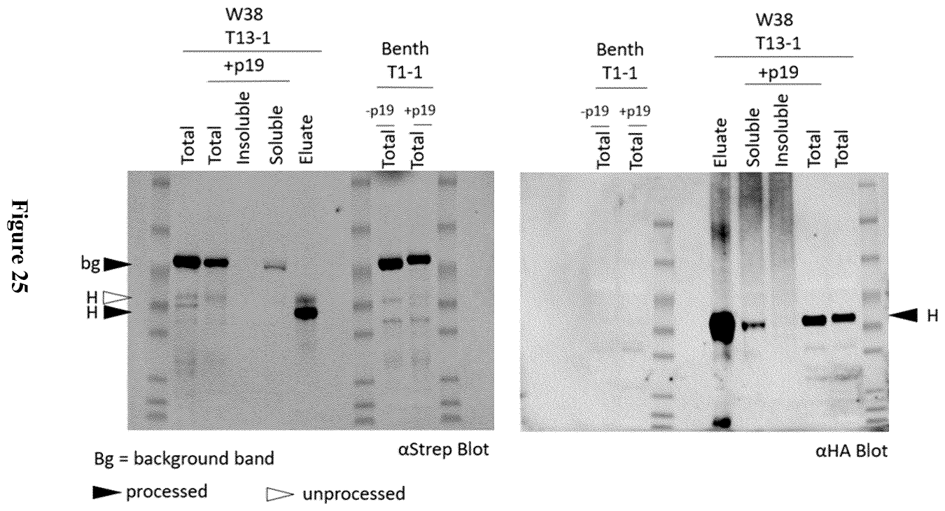

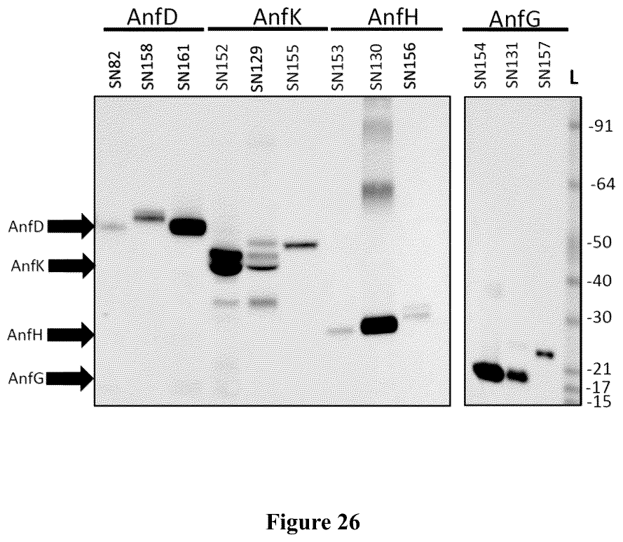

When the crude protein extracts were analysed by Western blotting using the anti-HA antibody, polypeptide bands were readily detected which matched the predicted sizes of the Anf polypeptides (FIG. 26). All of the individual mitochondrially-targeted polypeptides comprising the AnfD, AnfK, AnfH and AnfG sequences were expressed well and were seen after a short exposure (2 min) in the Western blot procedure. Each of the constructs SN161, SN130 and SN131 for the AnfD, AnfH and AnfG fusion polypeptides, respectively, each having the MTP-FAγ51 sequence, yielded a single dominant band on the blot at the molecular weight expected for a polypeptide processed by MPP within the MTP sequence. The bands in the adjacent lanes for the control polypeptides in each case confirmed that these bands were for processed polypeptides. It was concluded that these three fusion polypeptides were well expressed and processed efficiently in the mitochondria. The processing was subsequently confirmed by mass spectrometry. The sample from SN130 encoding MTP-FA751::HA::AnfH also showed a less distinct but nevertheless definite band at a higher molecular weight, at a size appropriate for a dimer of the polypeptide, despite the protein denaturation conditions used during the gel electrophoresis step.

The lanes for two of the AnfK constructs were more complicated with multiple bands. The AnfK cytoplasmic- and mitochondrially-targeted polypeptides produced from SN152 and SN129, respectively, showed additional bands detected by the HA antibody that were smaller than expected for cleavage within the MTP sequence, indicating that the AnfK polypeptide appeared to undergo additional proteolytic cleavage. The smaller polypeptides, about 4-6 kDa less in size, may also have arisen from premature transcription or translation termination. Despite this observation for AnfK, it was concluded that all four of the genetic constructs including the N-terminal MTP sequence had expressed the intended fusion polypeptide with processing, partially in the case of AnfK, to provide the desired mitochondrially localised Anf polypeptides.

Expression and Processing of Ant′ Fusion Polypeptides in Plant Cells from Multi-Gene Constructs

The first experiment described above had used single gene constructs for production of the individual Anf fusion polypeptides. The inventors now decided to test the expression of all four of the AnfD, AnfK, AnfH and AnfG fusion polypeptides from a single vector, with each of the Anf genes having its own 35S promoter and transcription terminator. This experiment aimed to test whether there were any interactions between the four Anf polypeptides when they were expressed together in the same plant cells, in particular looking for changes in the levels of accumulation of individual polypeptides or in their processing by MPP. To do this, a genetic construct was assembled having all four of the genes in a single T-DNA of the binary vector, with each gene having the MTP-FAγ51 sequence fused translationally to a HA epitope and followed by the Anf sequence. The same nucleotide sequences and amino acid sequences were used as for the single gene vectors SN161, SN129, SN130 and SN131. The resultant genetic construct was designated SL26. Two control constructs were also made, SL31 which encoded the four Anf fusion polypeptides each with the alanine-mutated MTP sequence for producing size markers for the unprocessed polypeptides (mFAγ51::HA::Anf), and SL36 which encoded the four fusion polypeptides lacking the MTP sequence (HA::Anf) as the size marker for processed polypeptides. Additionally, to aid in the identification of the multiple polypeptide bands in the Western blots, three additional vectors were made by stepwise deletion of one, two or three genes from SL26: SL27 had the AnfG gene deleted, SL28 had the AnfH and AnfG genes deleted, and SL29 had the AnfK, AnfH and AnfG genes deleted leaving only the AnfD gene. The multi-gene vectors and their constituent genes are listed in Table 20.

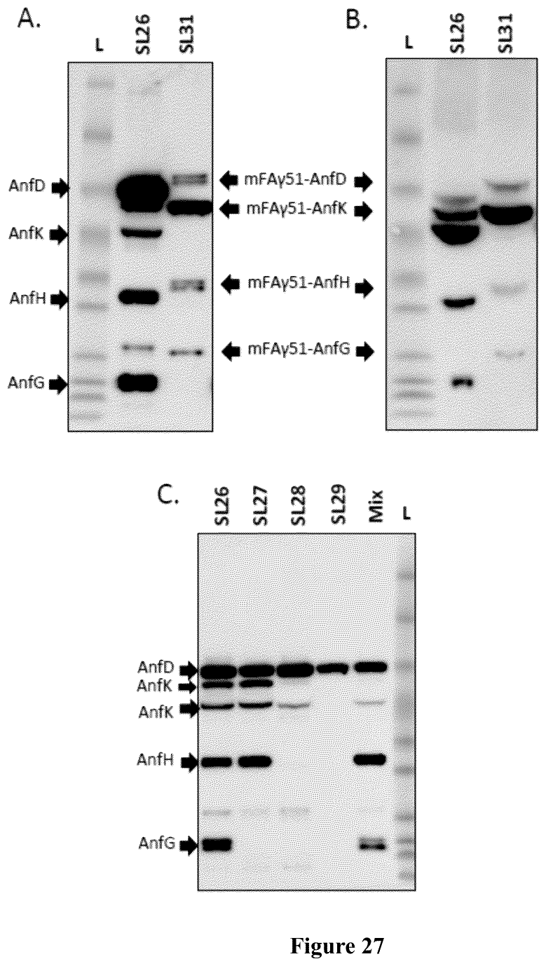

All of these multi-gene vectors were separately introduced into N. benthamiana leaves by the method described in Example 1. Proteins were extracted from the leaf tissues 4 or 5 days post-infiltration and analysed by Western blotting, as before. The results (FIG. 27) showed that all four of the Anf polypeptides fused to the MTP-FAγ51 and HA sequences were readily detected, being well expressed as single, strong bands. Furthermore, the AnfD, AnfH and AnfG fusion polypeptides having the N-terminal MTP-FAγ51 leader sequences were efficiently processed within the MTP sequence and the AnfK fusion polypeptide was partially processed, as evidenced by the comparison with the sizes of the corresponding polypeptides expressed from SL31 in the adjacent lane. This was confirmed in a separate experiment using the multi-gene construct SL36 which encoded the 4 HA::Anf polypeptides that did not have the MTP sequence, and so providing size markers in the Western blot procedure for the processed polypeptides from SL26. The Western blot for the extracts arising from the series of vectors SL26, SL27, SL28 and SL29 (FIG. 27, panel C) assisted in identifying the four polypeptides in the mixture, confirming their identity, as did a mixture of the four single gene vectors in the lane labelled Mix.

The levels of accumulation of the four Anf polypeptides could be compared when expressed from the multi-gene construct relative to the mixture of the single gene constructs. For the multi-gene construct, the AnfD fusion polypeptide accumulated at a greater level than the other three Anf polypeptides (FIG. 27, panel A), which was surprising considering that the NifD gene was the most difficult to express of the corresponding NifD, NifK and NifH genes for the Mo-nitrogenase (Allen et al., 2017). Furthermore, the AnfD polypeptide appeared to be full-length and there was no evidence of a secondary, cryptic cleavage site in AnfD, contrary to the observations with NifD from K. oxytoca (Examples 6 and 7).

TABLE 20
Listing of multi-gene vectors and the encoded polypeptides
for expression of Anf fusion polypeptides in plant cells.

| | | Predicted molecular | |
|---|---|---|---|
| | | weight (kDa) | |
| Plasmid | Encoded fusion polypeptides | Unprocessed | Processed |
| SL23 | MTP-FAγ51::AnfD::HA | 65.4 | 60.7 |
| | MTP-FAγ51::HA::AnfK | 58.1 | 53.5 |
| | MTP-FAγ51::HA::AnfH | 36.9 | 32.4 |
| | MTP-FAγ51::HA::AnfG | 22.2 | 17.7 |
| SL26 | MTP-FAγ51::HA::AnfG | 22.2 | 17.7 |
| | MTP-FAγ51::HA::AnfD | 65.3 | 60.7 |
| | MTP-FAγ51::HA::AnfK | 58.1 | 53.5 |
| | MTP-FAγ51::HA::AnfH | 36.9 | 32.4 |
| SL27 | MTP-FAγ51::HA::AnfD | 65.3 | 60.7 |
| | MTP-FAγ51::HA::AnfK | 58.1 | 53.5 |
| | MTP-FAγ51::HA::AnfH | 36.9 | 32.4 |
| SL28 | MTP-FAγ51::HA::AnfD | 65.3 | 60.7 |
| | MTP-FAγ51::HA::AnfK | 58.1 | 53.5 |
| SL29 | MTP-FAγ51::HA::AnfD | 65.3 | 60.7 |
| SL30 | MTP-FAγ51::HA::AnfG | 22.2 | 17.7 |
| | MTP-FAγ51::AnfD::Twin Strep | 69.2 | 64.5 |
| | MTP-FAγ51::HA::AnfK | 58.1 | 53.5 |
| | MTP-FAγ51::HA::AnfH | 36.9 | 32.4 |
| SL31 | mFAγ51::HA::AnfD | 64.6 | NA |
| | mFAγ51::HA::AnfK | 57.4 | NA |
| | mFAγ51::HA::AnfH | 36.3 | NA |
| | mFAγ51::HA::AnfG | 21.6 | NA |
| SL34 | MTP-FAγ51::HA::AnfG | 22.2 | 17.7 |
| | MTP-FAγ51::HA::AnfD | 65.3 | 60.7 |
| | CoxIV::Twin Strep::AnfK | 57.7 | 54.7 |
| | MTP-FAγ51::HA::AnfH | 36.9 | 32.4 |
| SL36 | HA::AnfD | 59.7 | NA |
| | HA::AnfK | 52.5 | NA |
| | HA::AnfH | 31.3 | NA |
| | HA::AnfG | 16.6 | NA |
| SL37 | MTP-FAγ51::HA::AnfG | 22.2 | 17.7 |
| | MTP-FAγ51::AnfD::HA | 65.4 | 60.7 |
| | CoxIV::Twin Strep::AnfK | 57.7 | 54.7 |
| | MTP-FAγ51::HA::AnfH | 36.9 | 32.4 |

### Confirmation of the Mitochondrial Localisation and MPP Processing

The processing of the MTP-FAγ51::HA::Anf fusion polypeptides was a clear indication to the inventors of the mitochondrial localisation of the four, processed Anf polypeptides expressed from SL26. This was further confirmed by enrichment of mitochondrial fractions obtained from the infiltrated leaf tissues, using the metaxin-mediated method as described in Example 13. This involved the addition of the genetic construct SN197 encoding a TwinStrep-mTurquoise-TEV recognition sequence-metaxin fusion polypeptide (SEQ ID NO:121) in A. tumefaciens to a mixture with the Agrobacterium containing SL26. The metaxin region of the polypeptide from SN197, when expressed transiently in the plant cells, was localised to the outer membrane of mitochondria (Lister et al., 2007). This exposed the N-terminal TwinStrep motif to the cytosol, allowing for the rapid purification of the marked mitochondria under gentle conditions using beads coated with anti-Strep antibody. This resulted in a considerable enrichment of mitochondrial proteins relative to non-mitochondrial proteins in the same cell.

To test this, a mixture of A. tumefaciens cultures containing SN197 in one strain and SL26 in another strain was introduced into N. benthamiana leaves. Infiltrated tissues were harvested 5 days later. These tissues were processed for mitochondrial isolation as described in Example 13. Proteins in the isolated mitochondria were then analysed by SDS-PAGE and Western blotting using the HA antibody for detection. All of the Anf polypeptides were readily detected in the mitochondrial fraction. The bands detected on the Western blot were consistent with the sizes of the processed AnfD, AnfK, Antes and AnfG polypeptides from SL26, indicating once again that the Anf polypeptides were localised to the plant mitochondria. A smaller band from the AnfK fusion polypeptide that was probably formed by an additional proteolytic cleavage (see above) was also enriched in the mitochondrial fraction, suggesting that the secondary cleavage was occurring in the mitochondria. The observation that the Anf polypeptides were processed was evidence that they were localised to the mitochondrial matrix.

The processing by cleavage within the MTP sequence was confirmed by LC-MS methods after tryptic digestion of proteins, using the methods described in Example 1. Protein bands were isolated from Coomassie stained gels after electrophoresis of protein extracts expressed from SL26. The protein identity in gel slices was confirmed through LC-MS and targeted MRM. The protein identities matched to Metaxin, AnfD, AnfK and Antes with at least 95% confidence. The AnfG protein was not identified in the Coomassie gel that underwent LC-MS detection, probably due to its low-level accumulation. All of the Anf proteins except for AnfK were detected with the intended N-terminus after efficient cleavage of the MTP. For the AnfK polypeptide, two N-terminal FAγ51 MTP targeted peptides were detected by the MRM at a low signal level indicating that partial MPP cleavage of the AnfK fusion polypeptide was occurring. This was consistent with the observations made with the Western blot analysis and confirmed that partial cleavage was occurring at the intended site within the MTP sequence by MPP.

Solubility of Mitochondrial Anf Fusion Polypeptides after Expression in Plant Cells

The inventors considered that, to be functional, the Fe-nitrogenase proteins should be produced in a soluble form to allow for the necessary protein to protein interactions and stability in the Fe-nitrogenase enzyme, as well as allowing the enzymes to interact with their substrates and cofactors. If the proteins were not in a soluble form, it could be an indication of improper protein folding or tight binding to the mitochondrial membranes and thereby detrimental to nitrogenase activity. Therefore, experiments were carried out to test whether the expressed Anf polypeptides were in a soluble form when produced in plant mitochondria. This was done by fractionating protein extracts into soluble (supernatant) and insoluble (pellet) forms using the method as described in Example 1.

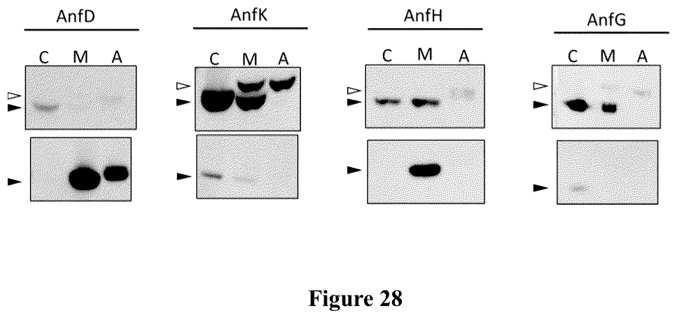

This was first done using the single gene constructs in N. benthamiana leaves, as before. Protein extracts for the soluble and insoluble proteins were prepared from leaves inoculated with the genetic constructs and analysed by Western blotting (FIG. 28). The Western blot showed that when the AnfD fusion polypeptide was targeted to the mitochondria in N. benthamiana leaves, it was essentially insoluble with only a very faint band visible in the soluble fraction (FIG. 28). The processed and unprocessed AnfK polypeptides were essentially present only in the soluble fraction, whereas the processed Antes polypeptide was only partially soluble. The mitochondrially-targeted AnfG polypeptide was only present in the soluble fraction, indicating that the processed AnfG fusion polypeptide was in a soluble form when expressed with the mitochondrial matrix of N. benthamiana.

The solubility of mitochondrially-targeted AnfD, AnfK, Antes and AnfG when co-expressed was tested in the following manner. The solubilities of the AnfD, AnfK, Antes and AnfG fusion polypeptides expressed from the multi-gene vector SL26 were compared to the solubilities of the polypeptides expressed from SL31. A mixture of Agrobacterium strains each containing a single-gene construct was also used to infiltrate the plants. The Western blot is shown in FIG. 27, panel B.

A surprising and unexpected result was observed with the multi-gene vector SL26. This time, some of the processed AnfD fusion polypeptide was clearly observed in the soluble fraction, indicating that the co-expression of the other Anf polypeptides had increased the solubility of at least some of the AnfD polypeptide. This suggested that either some of the AnfD polypeptide was being stabilised, perhaps through a protein to protein association of the AnfD polypeptide with one or more of the other Anf polypeptides, or increased folding was occurring of the AnfD polypeptide into its proper conformation. The possibility of protein-protein association was tested as described in the following Example.

The series of vectors SL26, SL27, SL28 and SL29 were used in a similar experiment to compare the solubility of the AnfD polypeptide when co-expressed with one, two or all three of the other Anf polypeptides. The fusion polypeptides expressed from multi-gene vectors SL26, SL27 and SL28 and the single gene vector SL29 were tested for polypeptide accumulation levels and for soluble and insoluble AnfD polypeptides. The results from the transient N. benthamiana leaf assay indicated that as the number of Anf genes reduced, so did the solubility of the AnfD polypeptide, especially in the absence of AnfK Therefore, it was concluded that the presence of AnfK, in particular, enhanced the solubility of the AnfD polypeptide.

Further confirmation of the solubility of the mitochondrially-localised Anf polypeptides was obtained by an affinity purification experiment using beads linked to anti-HA antibody. When the crude extracts were contacted with the beads and unbound proteins washed away before analysing the bound proteins, each of the AnfD (both processed and unprocessed forms), AnfK (both processed and unprocessed forms), AnfH (processed) and AnfG (processed) fusion polypeptides were recovered from the beads. The HA-enriched polypeptide bands observed on the Coomassie stained gels were excised and the polypeptides in the gel slices were analysed by LC-MS mass spectrometry. The bands present in the gels were of the correct sizes for both processed and unprocessed AnfD, AnfK and AnfG polypeptides. The polypeptides identified for AnfG included a potentially partially processed polypeptide having an extra amino acid at the N-terminal MTP cleavage site. This was consistent with the observation of two close migrating bands present for AnfG in the Western blot for extracts from SL26 (See FIG. 27, panel C). The band present for AnfH was for the processed size only, indicating efficient processing within the MTP sequence. The identity of the polypeptide bands was confirmed through the LC-MS analysis.

Several other multi-gene vectors were designed and made to test whether the position of the Ant′ gene on the multi-gene vector or the position of the HA epitope affected the protein expression, solubility or both. These vectors included constructs designated SL23, SL30, SL34 and SL37. The different positioning of the genes on the multi-gene vector did not appear to significantly impact the protein expression and solubility.

The vector SL26 is used to transform tobacco (N. tabacum), N. benthamiana and Arabidopsis thaliana plants, producing stably transformed plants which express the AnfD, AnfK, AnfH and AnfG polypeptides.

### Discussion

These experiments demonstrated that it was possible to express Anf genes encoding AnfD, AnfK, AnfG and AnfH fusion polypeptides and have them processed and localised in the mitochondria of plant cells. The polypeptides were proved to be cleaved at the intended site within the MTP sequence, in each case leaving a 9 amino acid “scar sequence” at the N-terminus of the fusion polypeptide. Mitochondrial localisation was also demonstrated in several different ways. Single and multi-gene constructs were introduced and expressed using a leaf assay with the plant N. benthamiana. The solubility of mitochondrially-localised Anf polypeptides was also tested. Solubility of AnfD increased with the use of multi-gene constructs to co-express AnfK, AnfH and AnfG.

### Example 19. Synergistic Interaction of Fe-Nitrogenase Polypeptides within Plant Leaf Mitochondria

In diazotrophic bacteria, the AnfD, AnfK and AnfG proteins form a heterohexamer complex that, with the required cofactors, constitutes the dinitrogenase enzyme (Davis et al., 1996; Zheng et al., 2018). This complex is the catalytic enzyme for reduction of dinitrogen. In order to be an active enzyme, this complex requires the FeFeco-factor and multiple Fe—S clusters.

The inventors designed and carried out several experiments to detect protein-protein interactions of the Anf polypeptides within plant mitochondria after expression from a multi-gene vector. To test this in a first experiment, a vector designated SL30 (Table 20) was designed and made that contained anfD, anfK, anfH and anfG genes, each expressed from its own 35S promoter and with the same transcription terminators as for SL26. The important modification relative to SL26 was that the AnfD fusion polypeptide of SL30 had a TwinStrep epitope fused to the C-terminus of AnfD to provide for purification of the AnfD polypeptide under gentle, non-denaturing conditions. SL30 still had the MTP-FAγ51 sequence fused to the N-terminus of AnfD for mitochondrial localisation. The AnfK, AnfH and AnfG fusion polypeptides encoded by SL30 had the MTP-FAγ51 sequence translationally fused at the N-terminus of the polypeptides followed by a HA epitope and then the Anf sequence, as for SL26. Each individual gene in SL30 retained its own 35S promoter and terminator, again as for SL26.

SL30 was introduced into A. tumefaciens and cultures of the transformed Agrobacteria were infiltrated into N. benthamiana leaves as before. Five days later, leaf samples were harvested and processed under ambient air conditions for extraction of soluble proteins into extraction buffer, using the same extraction buffer as in Example 14. The crude protein mixture was passed through a Strep-tactin XT affinity column under aerobic conditions. After washing the column with 10 column volumes of wash buffer (as per Example 14) to remove unbound proteins, the bound proteins were eluted with wash buffer containing 50 mM biotin, pH 7.2, and analysed by SDS-PAGE and Western blotting using Strep-tactin antibody for detection of the AnfD polypeptide and anti-HA antibody to detect any co-purifying Anf polypeptides having the HA epitope.

Extracted proteins were assessed by the Western blot method using Strep-tactin antibody. The analysis showed that purified AnfD polypeptide was present in the eluate and that it migrated at the molecular weight for the processed form (FIG. 26), indicating that the mitochondrially-targeted AnfD was processed, soluble and interacted with the Strep-tactin affinity media. When the Western blot was probed with the HA antibody, a faint but clearly visible band corresponding to the AnfK fusion polypeptide was observed, migrating at a speed consistent with a correctly MPP-processed isoform of AnfK. This indicated that the AnfK fusion polypeptide had been co-purified through association with the AnfD polypeptide. The AnfG polypeptide was not visible in the Western blot. There were also several bands of lower molecular weight on the Western blot that may have represented breakdown products of AnfD, which may have occurring post-extraction.

A second, analogous experiment was carried out in the same manner except that a new multi-gene construct, SL34, was made and used. With this construct, the TwinStrep epitope was fused to the AnfK sequence, between the MTP and AnfK sequences, and the AnfD polypeptide was the same as the one encoded by SL26 (Table 20) i.e with a HA epitope. This configuration was designed to test for the reverse capture and detection compared to the experiment with SL26, in that the AnfK polypeptide could be purified on the Strep-tactin column and the bound proteins analysed with the HA antibody for the presence of the other Anf polypeptides. The AnfK polypeptide encoded by SL34 contained a CoxIV MTP leader sequence with a fused Twin-strep at the N-terminus of AnfK, rather than the MTP-FAγ51. The AnfD, AnfH and AnfG fusion polypeptides encoded by SL34 each had the MTP-FAγ51 sequence translationally fused at the N-terminus of the polypeptide followed by the HA epitope. The CoxIV MTP has previously been shown to correctly target proteins to the mitochondrial matrix within N. benthamiana (Buren et al., 2017).

An A. tumefaciens culture containing SL34 was infiltrated into N. benthamiana leaves and leaf samples harvested 5 days later. The tissue samples were processed using the same experimental conditions for SL30 under ambient air, and the resultant crude protein extracts passed through the Strep-tactin column to purify the AnfK polypeptide containing the TwinStrep sequence. The eluate from the column was again analysed by SDS-PAGE and Western blotting using both the HA and Strep-tactin antibodies for detection of polypeptides having the HA- and TwinStrep-epitopes, respectively. The Western blot that was probed with Strep-tactin antibody showed the presence of purified AnfK polypeptide in the eluate, as intended, and the molecular weight of the polypeptide was consistent with it being the MPP-processed isoform. When the Western blot was further probed with the HA antibody, the presence of AnfD polypeptide was observed, indicating that the AnfD polypeptide had been co-purified with the AnfK polypeptide. The molecular weight of the AnfD was consistent with it being the MPP-processed isoform. AnfG was again not observed in the Western blot but was later detected at low signal intensities by LC-MS mass spectrometry. This experiment, like the previous experiment with SL30, demonstrated that the MPP-processed AnfD and AnfK polypeptides targeted to the mitochondrial matrix of the plant cells were associating together.

Another multi-gene vector was assembled, SL37 (Table 20), which encoded a fusion polypeptide having MTP-CoxIV and Twin-strep sequences fused at the N-terminus of AnfK, and AnfH and AnfG fusion polypeptides having the MTP-FAγ51 sequence translationally fused followed by a HA epitope at the N-terminus of the other Anf polypeptides. The AnfD polypeptide also had the MTP-FAγ51 MTP translationally fused to the N-terminus whereas the HA epitope was translationally fused at the C-terminus of the AnfD sequence. This construct was designed to test whether the AnfK polypeptide was associating with the full-length, processed AnfD polypeptide or possibly with a truncated AnfD product. This time, the protein extraction and processing were conducted under anaerobic conditions. The protein extract was passed through a Strep-tactin XT affinity column and then eluted, all under anaerobic conditions. The eluate was then analysed by SDS-PAGE and Western blotting with the HA and strep-tactin antibodies for detection.

The Western blot probed with the Strep-tactin antibody showed the presence of the processed AnfK polypeptide in the eluate. Furthermore, the Western blot probed with the HA antibody showed polypeptide bands corresponding in size to both processed and unprocessed AnfD polypeptides, with bands at lower molecular weights representing smaller AnfD products which were likely produced post-extraction. Polypeptide bands of the sizes for the AnfH and AnfG polypeptides were also observed in the eluate but at a much lower intensity than for AnfK or AnfD (FIG. 26).

The eluates produced from the SL34 and SL37 samples were analysed by LC-MS mass spectrometry and targeted MRM. Peptides from the AnfK, AnfD and AnfG polypeptides were detected in both eluates, with peptides from AnfH detected but only after anaerobic extraction.

As a negative control for the above experiments, to test the specificity of the detection, SL26, which encoded the AnfD, AnfK, AnfG and AnfH polypeptides, all fused to MTP-FAγ51 and having a HA epitope at the N-terminus (Table 20), was introduced into the N. benthamiana leaves. Leaf tissues were processed in the same manner as for SL30 and SL34 in aerobic conditions as described above. The only polypeptide band observed in the Strep-tactin probed Western blot of the protein extracts from SL26 was a relatively faint background band. There were no Anf polypeptide bands present in the eluate for either Strep-tactin or the HA probed Western blots. This control experiment demonstrated that the polypeptides containing the HA epitope observed on the Western blots were specifically from association of the AnfD and AnfK polypeptides.

Another multi-gene vector was assembled, SL93 (Table 22), having four genes encoding fusion polypeptides: one having MTP-CoxIV and Twin-strep sequences fused at the N-terminus of AnfG (MTP-CoxIV::TS::AnfG), and the other three separately encoding AnfH, AnfD and AnfK fusion polypeptides each having the MTP-FAγ51 sequence translationally fused followed by a HA epitope at the N-terminus. This construct was designed and made to test whether the AnfG polypeptide was associating with the AnfD polypeptide and/or with the AnfK polypeptide, by purification of the AnfG fusion polypeptide first of all by the Twin-Strep tag. In this experiment, the protein extraction and processing of the samples were conducted under anaerobic conditions. Following infiltration of N. benthamiana leaves with SL93 from Agrobacterium and preparation of extracts five days later, the protein extract from SL93 was passed through a Strep-tactin XT affinity column and then bound proteins eluted with biotin, all under anaerobic conditions. The eluate was then analysed by SDS-PAGE and Western blotting with the HA and Strep-tactin antibodies for detection, as before.

The Western blot probed with the Strep-tactin antibody showed the presence of the correctly processed AnfG polypeptide in the eluate. Furthermore, the Western blot of the same eluate probed with the HA antibody showed polypeptide bands corresponding in size to both processed and unprocessed AnfD and AnfK polypeptides after a 20 min exposure, although at a much lower intensity than for AnfG. Polypeptide bands of the sizes for the AnfH, AnfD and AnfK polypeptides were also observed in the total protein prior to the AnfG purification process. The eluate produced from the SL93 sample was analysed by LC-MS mass spectrometry and targeted MRM. Peptides from AnfG, AnfD and AnfK were detected within the eluate. Clearly, the AnfG fusion polypeptide was associating with the AnfD and AnfK polypeptides in the plant mitochondria, indicating that they were all in sufficiently soluble form to be able to form a complex.

### Discussion

These experiments demonstrated for the first time the production of the distinct Fe-nitrogenase (Anf) proteins within a eukaryotic environment, specifically in plant mitochondria. The multi-gene constructs and differential epitope tagging in these experiments were used to show an association between AnfD and AnfK fusion polypeptides targeted to the plant mitochondrial matrix, and furthermore with AnfG fusion polypeptide. These results demonstrated that it was possible to produce multiple Anf polypeptides and localise them within the plant mitochondria in a soluble form to allow protein complexes. Moreover, co-expression of multiple Anf genes (AnfK, AnfH, AnfG) from a single vector led to an increase in AnfD polypeptide solubility, even though it was still only partially soluble.

When processed under aerobic conditions, some of the purified AnfD polypeptide co-purified with the AnfK polypeptide. A reverse experiment was conducted under aerobic conditions where AnfK was translationally fused to the TwinStrep epitope, where the other Anf polypeptides were all fused to a HA epitope. Some AnfD protein co-purified with the AnfK polypeptide as well as low amounts of the AnfG protein. When an analogous experiment was conducted under anaerobic conditions, again only low amounts of the AnfG protein were detected, indicating that the AnfD, AnfK and AnfG polypeptides were interacting within the soluble fraction of the mitochondria to form a complex. The detection of AnfG and AnfD along with AnfK as it was being purified indicated a three-way association. It was also demonstrated that AnfG co-purified with AnfD-AnfK under anaerobic conditions. The predicted structure of the FeFe nitrogenase has the AnfG polypeptide physically interacting with the surface of AnfD (Sippel and Einsle, 2017; Zheng et al., 2018). Interestingly, in one experiment when the extraction was conducted under anaerobic conditions, small amounts of the AnfH protein were also found within the eluate.

The AnfG protein was observed at a lower abundance in the pull-down experiments relative to AnfD and AnfK A band at the correct size for AnfG was visible after a longer exposure. The lesser abundance of AnfG may indicate that the optimal ratio of the subunits for the Fe-nitrogenase heterohexamer has not yet been achieved.

The inventors concluded from these experiments that the association of the AnfD and AnfK polypeptides and the three-way association of AnfD, AnfK and AnfG demonstrated the potential for using these Fe-nitrogenase components in plant mitochondria for nitrogenase engineering.

### Example 20. Production of a Translational Fusion Between AnfD and AnfK Targeted to Plant Mitochondria

Although a crystal structure for the Fe-nitrogenase has not been reported, it has been predicted that the AnfD, AnfK and AnfG subunits of the Fe-dinitrogenase in nitrogen fixing bacteria that have the Fe-nitrogenase are in a 1:1:1 stoichiometric ratio (Hu & Ribbe, 2015; Zheng et al., 2018). That ratio for the AnfD, AnfK and AnfG polypeptides may be important for the optimal function of the Fe-nitrogenase and may influence solubility of the AnfD component. As described in this Example, a predicted structural model for the Fe-nitrogenase was developed. The model was used to design an oligopeptide linker of an appropriate length to join the C-terminus of AnfD to the N-terminus of AnfK and thereby generate a translational fusion of AnfD and AnfK The length of the linker was designed to allow for the correct folding of the protein complex, based on the predicted structural model. Genetic constructs to express the fusion polypeptide were made and tested. The fusion polypeptide had an MTP sequence to localise it to the mitochondrial matrix.

### Generation of a Structural Model for the Fe-Nitrogenase

To design an AnfD::linker::AnfK fusion polypeptide, a homology model was created for the AnfDKHG complex based on the A. vinelandii V-nitrogenase crystal structure PDB ID: 5N6Y (Sippel and Einsle, 2017). This was used since no Fe-nitrogenase crystal structure had been reported, and the V-nitrogenase was thought to be the nearest in sequence homology. Homology models were constructed using SWISS-MODEL (swissmodel.expasy.org/) for each of the wild-type A. vinelandii AnfD and AnfK polypeptides (SEQ ID NOs:216 and 217) using the respective monomers from the PDB ID: 5N6Y α₂β₂-heterodimer as templates. The AnfD model had the C-terminal 31 residues of the wild-type sequence missing (NSETLRQYTGGYDSVSKLREREYPAFERKVG, SEQ ID NO:197), and the AnfK model had two N-terminal amino acids missing (PH). The full heterodimer was constructed using the matchmaker function in Chimera to superpose the AnfD and AnfK homology models onto the native 5N6Y α₂β₂-heterodimer, after which the above-mentioned missing residues were manually added to the model using Discovery Studio 2018 (Dassault Systèmes BIOVIA, San Diego). The 31 amino acid residues at the C-terminus of the AnfD monomer were added as α-helices, so as to take a conservative approach to the overall length of this section. AnfD was 36 residues longer at the C-terminus than the VnfD structure upon which it was built, so it is not possible to say with certainty what conformation this additional sequence would take. Therefore, the modelling took the shortest option available for the 31 amino acids that were not initially constructed during the generation of the homology model.

The entire α₂β₂ heterodimer model, without cofactors, was prepared for molecular dynamics using the Xleap module of AMBER18 by solvating in a periodic water box (TIP3P, truncated octahedron, 12.0 Å minimum boundary distance from the solute) and neutralising with Na⁺ ions (frcmod.ionsjc tip3p). The system was subjected to energy minimisation with Amber18 using 25,000 steps of steepest descent followed by 25,000 steps of conjugate gradient, followed by 20 ns of molecular dynamics using AMBER18. The protein was treated with the ff14SB forcefield and the simulation was conducted at 298 K (NVT ensemble) using a 12.0 Å cutoff with long range interactions treated with the particle mesh Ewald summation. The purpose of the simulation was to identify potential regions of high strain and any other potentially detrimental features, hence 20 ns was sufficient for this task. The trajectory was analysed using VMD (hwww.ks.uiuc.edu/). The α-helices constructed for the 31 residues added at the C-terminus of AnfD retained their structure over the course of the trajectory, suggesting this could possibly be their native conformation, although more extensive dynamics simulations would be required for further corroboration. The added residues and linkers relaxed early in the simulation with no apparent adverse interactions with the rest of the structure.

It was predicted from the model that a peptide linker joining the C-terminus of AnfD to the N-terminus of AnfK could create a fusion protein that retained its overall structure and therefore keep its function. An initial linker peptide sequence of 16 amino acids designated linker16 was used for modelling, having the amino acid sequence GGGSGGGSGGGSGGGS (SEQ ID NO:198), expected to provide a disordered linker. The homology models predicted that an oligopeptide of at least 16 amino acids in length could span the required distance. The linker of 16 amino acids was therefore added in extended conformation and then relaxed with a series of rough geometry optimisations in Discovery Studio.

Coordinates of the AnfDK fusion dimer were generated from the final frame of the 20 ns molecular dynamics simulation and this structure was superposed with PDB ID: 5N6Y in order to generate starting positions for a homology model of AnfG, which was generated with SWISS-MODEL using a VnfG monomer from 5N6Y as a template. Once the Anf(DKG)₂ model was constructed, it was superposed with the NifDKH model from PDB ID: 1N2C to generate starting positions for the AnfH homology models, which were constructed in SWISS-MODEL using a NifH monomer from PDB ID: 1N2C as a template. Prior to molecular dynamics, conducted as described above, the AnfG and AnfH dimer structures were manually positioned slightly away from their interfaces with the AnfD-AnfK fusion structures to relieve steric clashes that arose from artefacts of the superposition.

The amino acid sequence of the synthetic fusion polypeptide with the linker16 is provided as SEQ ID NO:199. The modelled structure is represented in FIG. 29.

For detection purposes, a HA epitope having the sequence YPYDVPDYA (SEQ ID NO:115) was added into the middle of the 16 amino acid linker, to provide a 26-amino acid sequence GGGGSGGGSYPYDVPDYAGGGSGGGS (SEQ ID NO:200), designated herein as “linker26(HA)”. The HA epitope was not included in the minimisation or the molecular dynamics. The fusion polypeptide with this linker26(HA) between and joining the AnfD and AnfK sequences and with no N-terminal MTP sequence (SEQ ID NO:201), or MTP-FAγ51 (SEQ ID NO:202), MTP-CoxIV (SEQ ID NO:203), mFAγ51 (SEQ ID NO:204) or a 6×His sequence (SEQ ID NO:205) fused to the N-terminus of the fusion polypeptide was in each case predicted to allow the AnfD, AnfK, AnfG and AnfH polypeptides to associate properly with no predicted adverse effects on the native structure. In these designs, the AnfG protein was not included in this linker design as both N- and C-terminals of AnfG were buried close to the surface of AnfD and seemed unlikely to tolerate any linker extensions. It has also been demonstrated that both AnfG and AnfK do not tolerate a C-terminal amino acid extension (Yang et al., 2018), which was consistent with the homology-based model of the Fe-nitrogenase developed as described above.

Constructs used in this Example are summarised in Table 21.

TABLE 21
Listing of genetic constructs used in this Example.

| | | Predicted molecular | |
|---|---|---|---|
| | | weight (kDa) | |
| Plasmid | Encoded polypeptide | Unprocessed | Processed* |
| SN272 | MTP-FAγ51::HA::AnfD::Linker26(HA)::AnfK | 118 | 113 |
| SN273 | MTP-CoxIV::TwinStrep::AnfD::Linker26(HA)::AnfK | 118 | 115 |
| SN274 | mFAγ51::HA::AnfD::Linker26(HA)::AnfK | 117 | NA |
| SN275 | HIS::AnfD::Linker26(HA)::AnfK | 112 | NA |
| SN161 | MTP-FAγ51::HA::AnfD | 65.3 | 60.7 |
| SN129 | MTP-FAγ51::HA::AnfK | 58.1 | 53.5 |
| SL26 | MTP-FAγ51::HA::AnfG | 22.2 | 17.7 |
| | MTP-FAγ51::HA::AnfD | 65.3 | 60.7 |
| | MTP-FAγ51::HA::AnfK | 58.1 | 53.5 |
| | MTP-FAγ51::HA::AnfH | 36.9 | 32.4 |
| SL28 | MTP-FAγ51::HA::AnfD | 65.3 | 60.7 |
| | MTP-FAγ51::HA::AnfK | 58.1 | 53.5 |
| SN129 | MTP-FAγ51::HA::AnfK | 58.1 | 53.5 |
| SN161 | MTP-FAγ51::HA::AnfD | 65.3 | 60.7 |

### Synthesis and Testing of Genetic Constructs to Express the AnfD-Linker-AnfK Polypeptide in Plant Cells

A DNA sequence encoding the AnfD::Linker26(HA)::AnfK protein coding region was chemically synthesised and used to make a set of genetic constructs through GoldenGate protocols, using the A. vinelandii amino acid sequences for AnfD and AnfK The protein coding region was codon-optimised for plant expression. Expression of the gene encoding the fusion polypeptide in plant cells was under the control of the 35S promoter and Nos3′ polyadenylation region/transcription terminator (Table 21). For mitochondrial targeting, a sequence encoding MTP-FAγ51::HA was added upstream of the AnfD::Linker26(HA)::AnfK protein coding region so that, when transcribed and translated, the MTP and HA amino acid sequences were translationally fused to the AnfD::linker26(HA)::AnfK polypeptide as a single translational product. The genetic construct encoding this fusion polypeptide was designated SN272. The amino acid sequence of the full-length fusion polypeptide encoded by SN272 is provided as SEQ ID NO:202. A second vector designated SN273 was made which encoded an identical polypeptide except that an MTP sequence from a CoxIV gene with a TwinStrep sequence (Buren et al., 2017) was substituted for the MTP-FAγ51 sequence. The amino acid sequence of the full-length fusion polypeptide encoded by SN273 is provided as SEQ ID NO:203. To provide molecular weight markers to detect processing of the translation product within mitochondria, two genetic constructs were made as controls. The first (SN274) lacked the MTP-FAγ51 sequence and therefore would be targeted to the cytoplasm. The second (SN275) had a mutated MTP-FAγ51 sequence that prevented cleavage by MPP, designated mFAγ51. The amino acid sequences of the fusion polypeptides encoded by SN274 and SN275 are provided as SEQ ID NOs:204 and 205.

These vectors were separately introduced into N. benthamiana leaves using the Agrobacterium-mediated methods described in Example 1. As further controls, vectors expressing individual Anf proteins in various combinations, SL26, SL28, SN161 and SN129, were also infiltrated into N. benthamiana leaves. Leaf tissues were harvested 4 days post-infiltration and processed for total, soluble and insoluble protein fractions as described in Example 1. The resulting protein fractions were analysed by SDS-PAGE and Western blotting using the HA epitope for detection.

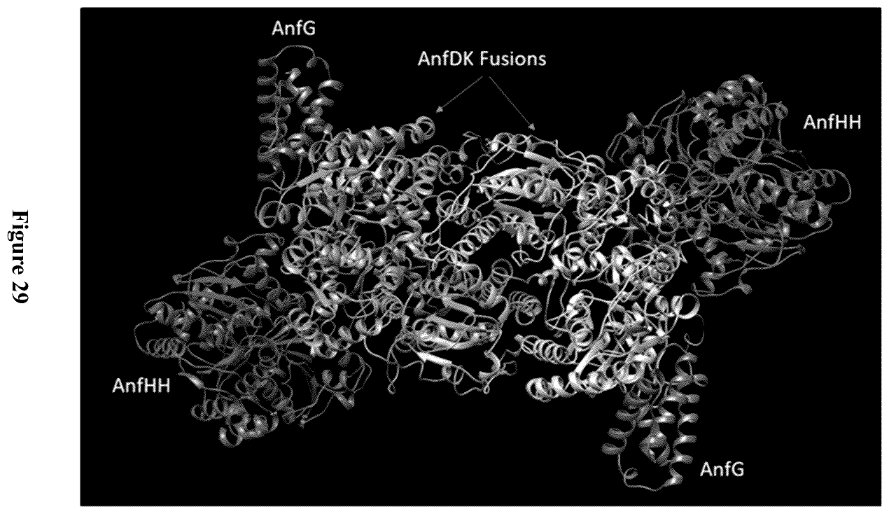

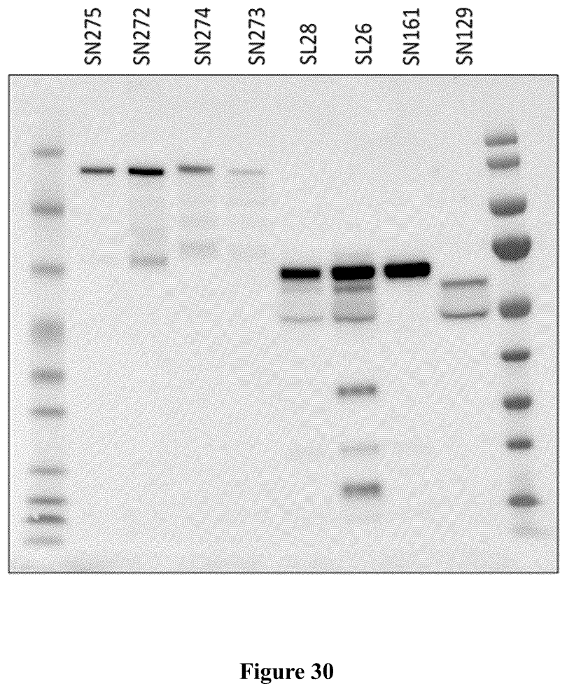

The Western blots revealed that all of the AnfD::linker26(HA)::AnfK fusion polypeptides were readily detected in the total protein fractions isolated from the N. benthamiana leaves (FIG. 30). The molecular weight of the main polypeptide band from each construct was consistent with the predicted size of the polypeptides in the range 110-120 kDa (see Table 21). The predicted size of the full-length (unprocessed) MTP-FAγ51::HA::AnfD::Linker26(HA)::AnfK fusion polypeptide was approximately 118 kDa. The processed polypeptide after cleavage was predicted to be approximately 113 kDa, which could be distinguished from the unprocessed polypeptide by their different mobilities on the SDS-PAGE gels and in the Western blots. The molecular weight of the polypeptide detected on the Western blots (FIG. 30) matched the control polypeptide encoded by SN275 which represented the processed form, indicating that the MTP-FAγ51::HA::AnfD::Linker26(HA)::AnfK polypeptide from SN272 had been efficiently imported into the mitochondria import and cleaved within the N. benthamiana cells. Likewise, the polypeptide band generated from the construct SN273 encoding the MTP-CoxIV::TwinStrep::AnfD::Linker26(HA)::AnfK fusion polypeptide also appeared to be efficiently and correctly processed. The MTP-FAγ51::HA::AnfD::Linker26(HA)::AnfK fusion polypeptide from SN272 had two HA epitopes whereas the MTP-CoxIV::TwinStrep::AnfD Linker26(HA)::AnfK fusion polypeptide from SN273 had only one, so the former polypeptide may have been detected more efficiently per polypeptide in these Western blots.

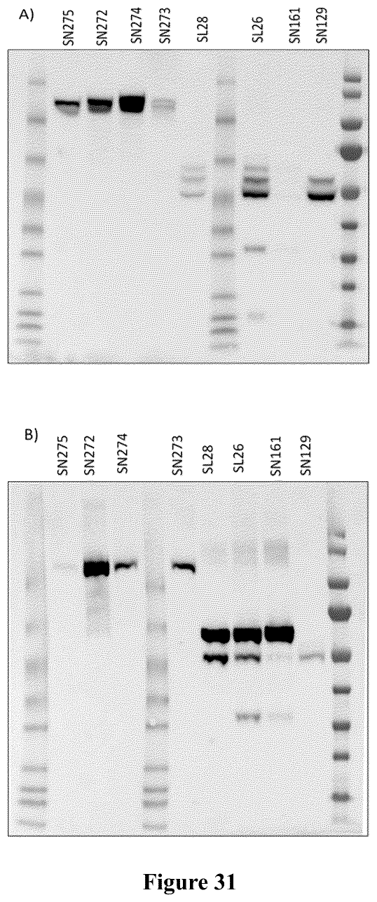

The Western blots of the soluble and insoluble fractions indicated that expression of mitochondrially-targeted AnfD by itself from SN161 resulted in a predominantly insoluble polypeptide (FIG. 31, panel A), with only very faint bands visible. However, the solubility of the AnfD polypeptide was increased when the same AnfD gene was co-expressed with AnfK from SL28 and further improved when the AnfD gene was co-expressed with AnfK, AnfH and AnfG from SL26. In each case where the AnfD and AnfK genes were co-expressed, the AnfD and AnfK polypeptides were detected in different abundances in the soluble fraction, despite the genes being expressed from the same T-DNA. In contrast, the translational fusion of AnfD and AnfK in the form of a MTP::HA::AnfD::Linker26(HA)::AnfK fusion polypeptide, targeted to the mitochondria as in SN272 and SN273, necessarily provided the ideal stoichiometric ratio for AnfD and AnfK polypeptides as 1:1. The inventors concluded that the fusion polypeptide using the linker sequence had at least this advantage relative to expression of the polypeptides from separate genes, even when the two genes were linked on one T-DNA.

The polypeptides resulting from processing of the MTP::HA::AnfD::Linker26(HA)::AnfK polypeptides expressed from SN272 and SN273 were detected in both the soluble and insoluble fractions of the plant extracts (FIG. 31, panels A) and B)). Since the addition of genes expressing AnfH and AnfG targeted to the mitochondria increased the solubility of mitochondrially-targeted AnfD, further experiments co-expressed mitochondrially-targeted MTP::HA::AnfD::Linker26(HA)::AnfK together with mitochondrially-targeted AnfH and AnfG, see below.

The polypeptide resulting from processing of the MTP-FAγ51::HA::AnfD::Linker26(HA)::AnfK fusion polypeptide was purified after expression of the gene from SN272, using the HA epitope in an affinity based purification method. The purified protein is subjected to proteomics analysis to confirm that the N-terminal sequence is as expected for the cleavage by MPP.

The genetic construct SN272 was a binary vector suitable for producing stably transformed plants by Agrobacterium-mediated transformation with the addition of a selectable marker gene. The gene encoding the fusion polypeptide was therefore excised and inserted into a binary vector containing a suitable selectable marker gene, resulting in the binary vector SL79. That vector was used to produce stably transformed Arabidopsis thaliana plants and can be used to transform other plants, for example tobacco and N. benthamiana plants. The fusion polypeptide was demonstrated to be expressed, cleaved within the MTP sequence at the intended site by MPP, and demonstrated to be present in the mitochondria (see below). At least some of the processed fusion polypeptide is present in the soluble fraction.

### Example 21. Production of Anf and Nif Proteins Required for the Fe-Nitrogenase within Plant Cells with Mitochondrial Targeting

### Introduction

A minimum of ten genes encoding Anf and Nif proteins was reported to be required to constitute Fe-nitrogenase in the bacterium E. coli (Yang et al., 2014), namely 4 structural Anf genes encoding the AnfD, AnfK, AnfH and AnfG polypeptides and 6 so-called accessory Nil genes encoding the NifV, NifS, NifU, NifJ, NifF and NifB polypeptides. The sequences for the Anf polypeptides were based on the nitrogen fixing bacterium A. vinelandii and, for the other Nif polypeptides, on the bacterium K. oxytoca. Expression of the set of ten genes in E. coli produced a functional Fe-nitrogenase although with low activity (Yang et al., 2014).

Based on the data described in the earlier Examples herein showing the production of Anf and Nif fusion polypeptides in plant mitochondria in soluble form, the present inventors decided to attempt to engineer plant cells to produce the minimum set of genes for producing Fe-nitrogenase, targeting the gene products to the mitochondrial matrix in the plant cells.

### Results

The set of gene products that was selected for a series of experiments included AnfD, AnfK, AnfG and AnfH polypeptides based on the diazotroph A. vinelandii (Av), and 6 Nif proteins, namely NifF, NifJ, NifS and NifU based on K. oxytoca (Ko), NifV from A. vinelandii (AvNifV) and NifB from Methanocaldococcus infernus (MiNifB). Genetic constructs were designed and made to express the polypeptides in N. benthamiana leaves with targeting to the mitochondrial matrix through translational fusion of N-terminal MTP sequences, in analogous fashion to the genetic constructs described in the earlier Examples. The nucleotide sequences for expressing the fusion polypeptides were codon optimised for expression in plant cells, as before. Two different MTP sequences were used, namely MTP-FAγ51 and MTP-CoxIV, for mitochondrial targeting of the fusion polypeptides. The polypeptides that had MTP-FAγ51 had a HA epitope fused at either the N- or C-terminus, whereas the polypeptides that had MTP-CoxIV had a TwinStrep epitope inserted between it and the Anf/Nif polypeptide. For expression in N. benthamiana, each gene was under the control of a 35S promoter and nos 3′ polyadenylation region/transcriptional terminator. These nucleotide sequences were upstream and downstream of each protein coding region, respectively. The constructs were assembled using the Golden Gate methods, as before.

The multi-gene constructs SL42 and SL43 were made using these principles and methods. The vectors each had five different, separate genes linked in one T-DNA (Table 22). SL42 had genes encoding fusion polypeptides which included the KoNifS, KoNifU, KoNifJ, KoNifF and MiNifB sequences, each with its own MTP and epitope sequences translationally fused. SL43 had genes encoding fusion polypeptides which included AvAnfD, AvAnfK, AvAnfH, AvAnfG and AvNifV sequences, again each having its own MTP and epitope sequences. The AvNifV sequence was selected out of the many available NifV sequences on the basis of the expression, processing and solubility data and evidence of homocitrate production by AvNifV targeted to plant mitochondria as described in Example 15.

TABLE 22
Single and multi-gene genetic constructs encoding components
of Fe-nitrogenase for expression in plant cells.

| | | Predicted molecular | |
|---|---|---|---|
| | | weight (kDa) | |
| Plasmid | Encoded fusion polypeptides | Unprocessed | Processed |
| SL42 | MTP-FAγ51::KoNifF::HA | 26.1 | 21 |
| | MTP-FAγ51::KoNifJ::HA | 135 | 130 |
| | MTP-FAγ51::KoNifS::HA | 50.3 | 45 |
| | MTP-FAγ51::KoNifU::HA | 36 | 31 |
| | MTP-CoxIV::TwinStrep::MiNifB | 41.6 | 38 |
| SL43 | MTP-FAγ51::HA::AvAnfG | 22.2 | 17.7 |
| | MTP-FAγ51::HA::AvAnfD | 65.3 | 60.7 |
| | MTP-CoxIV::TwinStrep::AvAnfK | 57.7 | 54.7 |
| | MTP-FAγ51::HA::AvAnfH | 36.9 | 32.4 |
| | MTP-FAγ51::HA::AvNifV | 48.4 | 43.8 |
| SL48 | MTP-FAγ51::HA::AvAnfG | 22.2 | 17.7 |
| | MTP-CoxIV::TwinStrep::AvAnfD::Linker26(HA)::AvAnfK | 118 | 115 |
| | MTP-FAγ51::HA::AvAnfH | 36.9 | 32.4 |
| | MTP-FAγ51::HA::AvNifV | 48.4 | 43.8 |
| SL49 | MTP-FAγ51::KoNifF::HA | 26.1 | 21 |
| | MTP-FAγ51::KoNifJ::HA | 135 | 130 |
| | MTP-FAγ51::KoNifU::HA | 36 | 31 |
| | MTP-FAγ51::HA::MiNifB | 41.9 | 37.3 |
| SN254 | MTP-FAγ51::HA::AvNifV | 48.4 | 43.8 |
| SL50 | MTP-FAγ51::HA::AvAnfG | 22.2 | 17.7 |
| | MTP-CoxIV::TwinStrep::AvAnfD::Linker26(HA)::AvAnfK | 118 | 115 |
| | MTP-FAγ51::HA::AvAnfH | 36.9 | 32.4 |
| | MTP-FAγ51::HA::AvNifV | 48.4 | 43.8 |
| | MTP-FAγ51::HA::AvFdxN | 16.4 | 11.9 |
| SL54 | MTP-FAγ51::KoNifF::HA | 26.1 | 21 |
| | MTP-FAγ51::KoNifJ::HA | 135 | 130 |
| | MTP-FAγ51::KoNifS::HA | 50.3 | 45 |
| | MTP-FAγ51::KoNifU::HA | 36 | 31 |
| | MTP-FAγ51::KoNifB::HA | 58 | 53 |
| SN192 | MTP-FAγ51::KoNifB::HA | 58 | 53 |
| SL78 | MTP-FAγ51::KoNifF::HA | 26.1 | 21 |
| | MTP-FAγ51::KoNifJ::HA | 135 | 130 |
| | MTP-FAγ51::KoNifS::HA | 50.3 | 45 |
| | MTP-FAγ51::KoNifU::HA | 36 | 31 |
| | MTP-FAγ51::HA::MiNifB | 41.9 | 37.3 |
| SL79 | MTP-CoxIV::TwinStrep::AvAnfD::Linker26(HA)::AvAnfK | 118 | 115 |
| | MTP-FAγ51::HA::AvAnfG | 22.2 | 17.7 |
| | MTP-FAγ51::HA::AvAnfH | 36.9 | 32.4 |
| | MTP-FAγ51::HA::AvNifV | 48.4 | 43.8 |
| | MTP-FAγ51::HA::FdxN | 16.4 | 11.9 |
| SL29 | MTP-FAγ51::HA::AvAnfD | 65.3 | 60.7 |
| SL93 | MTP-CoxIV::TwinStrep::AvAnfG | 21.9 | 18.9 |
| | MTP-FAγ51::HA::AvAnfD | 65.3 | 60.7 |
| | MTP-FAγ51::HA::AvAnfK | 58.1 | 53.5 |
| | MTP-FAγ51::HA::AvAnfH | 36.9 | 32.4 |

### Production of the Fusion Polypeptides in Plant Cells

Cultures of A. tumefaciens containing SL42 were infiltrated into 5-week-old N. benthamiana leaves as described in Example 1. Four to five days post infiltration, leaf samples were harvested. Total, soluble and insoluble protein fractions were extracted, as follows. For testing the solubility of plant-expressed polypeptides, the leaf tissue was ground in ice-cold extraction buffer (100 mM Tris pH 8.0, 150 mM NaCl, 0.25 M mannitol, 5% (v/v) glycerol, 1% (v/v) Tween 20, 1% (w/v) PVP, freshly-added 2 mM TCEP, 0.2 mM PMSF and 10 μM leupeptin) and transferred to a microfuge tube. The sample was centrifuged at 20,000×g for 5 min to divide the sample into soluble (supernatant) and insoluble (pellet) fractions. The supernatant was transferred to a fresh microfuge tube and centrifuged again at 20,000×g for 5 min to remove any remaining insoluble material. The insoluble fraction was washed by resuspension of the pellet in 300 μL extraction buffer, with dispersal by repeated strokes of the pipette, and centrifuged at 20,000×g for 5 min, discarding the supernatant. This washing step was repeated twice more, removing any remaining soluble protein from the insoluble fraction. Samples were then analysed by SDS-PAGE and Western blotting using anti-HA and anti-Strep antibodies. The anti-HA antibody (Monoclonal Anti-HA, Sigma) was used at 1:5000 dilution, and the anti-Strep/HRP conjugate antibody (Strep-MAB-conjugate HRP, IBA) was used at 1:10,000 dilution.

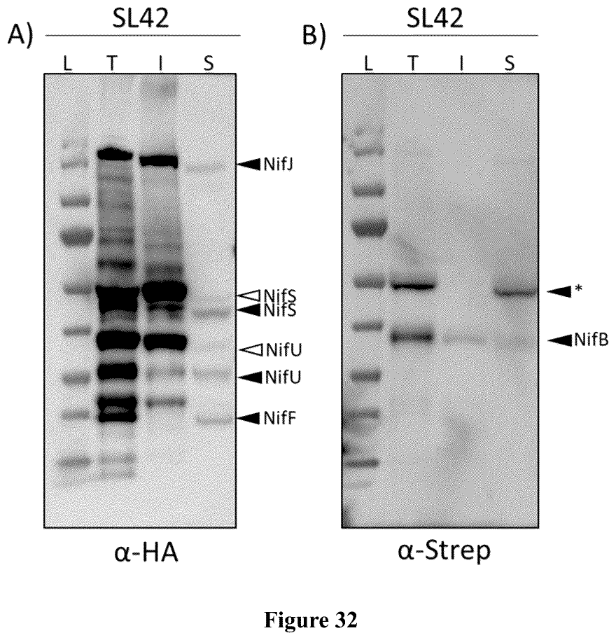

The Western blot analysis for SL42 (FIG. 32) showed that all five polypeptides were readily detected with the appropriate antibodies, each showing polypeptide bands present in the soluble protein fraction. The NifJ fusion polypeptide appeared to be entirely processed by MPP, whereas the NifU, NifS and NifF polypeptides were present as both processed and unprocessed forms, indicating less efficient cleavage by MPP. The NifJ, NifU, NifS, NifF and NifB polypeptides were present in both the soluble and insoluble fractions. The NifB polypeptide, which was translationally fused at the N-terminus with the MTP-CoxIV-Twin-strep sequence, was visible when the anti-Strep antibody was used for detection (FIG. 31, Panel B).

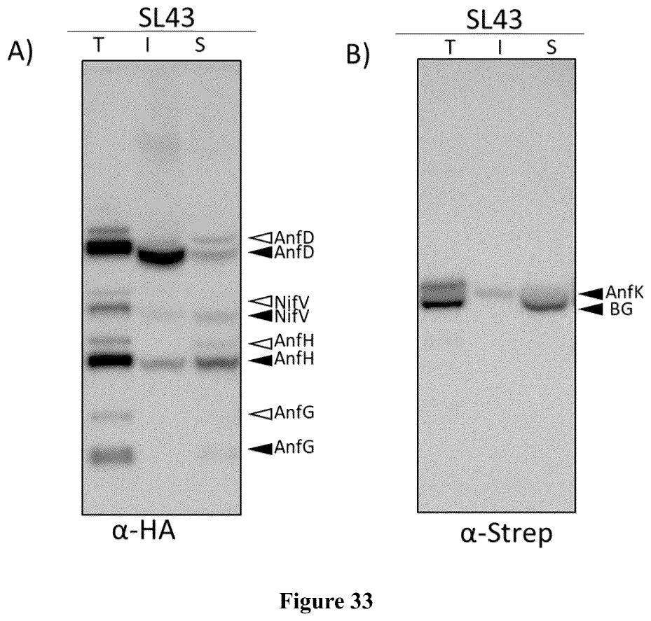

The Western blot analysis for SL43 (FIG. 33) also showed that all five of the encoded polypeptides were readily detected with the appropriate antibodies, each showing polypeptide bands present in the soluble protein fraction. Importantly, the processed AnfD, AnfK and AnfH fusion polypeptides were all observed in the soluble fraction. They were also observed in the insoluble fraction, indicating partial solubility for these three fusion polypeptides. This result was significantly better than had been observed with the expression of the corresponding genes from single-gene vectors. The AnfD, AnfG, AnfH and NifV fusion polypeptides all appeared to be partially cleaved by MPP, each showing bands for the processed and unprocessed forms. The AnfK fusion polypeptide appeared to be efficiently processed.

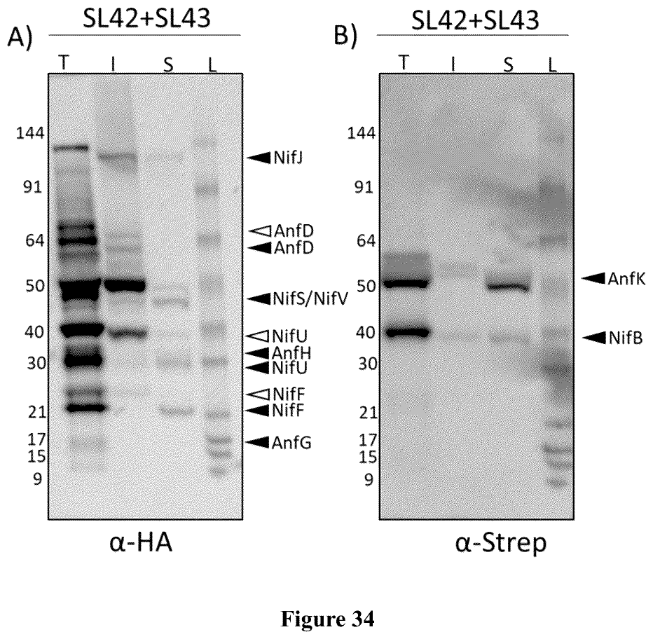

Next, the A. tumefaciens cultures containing SL42 and SL43 were mixed and infiltrated into N. benthamiana leaves as described before. This experiment therefore introduced all 4 AvAnf genes and all six of the Nif genes, 10 genes in combination. The surprising and significant result observed in the Western blot (FIG. 34) was that all 10 polypeptides were readily detected. Moreover, all 10 polypeptides were present in the soluble fraction, some exhibiting efficient processing by MPP. Several of the polypeptides were visible with 2 bands, with the upper band representing unprocessed polypeptide and the lower band representing the MPP-cleaved polypeptide and demonstrating mitochondrial import. The unprocessed polypeptide band was visible for the proteins AnfD, NifV, NifU and NifF as well as a band present at the predicted size for cleaved polypeptides.

### Association of AnfD and AnfK in Plant Cells

The multi-gene vectors, SL43 and SL49 (Table 22) were infiltrated into 5-week-old N. benthamiana plants, separately and in combination. SL43 encoded a fusion polypeptide having four separate genes encoding AnfD, AnfH, AnfG and NifV polypeptides each with the MTP-FAγ51 sequence translationally fused followed by a HA epitope at the N-terminus of the Nif polypeptides, and a fifth gene encoding MTP-CoxIV and Twin-strep sequences fused at the N-terminus of AnfK SL49 encoded NifJ, NifF and NifU fusion polypeptides with the MTP-FAγ51 sequence translationally fused at the N-terminus of the Nif polypeptides followed by a HA epitope at the C-terminus, and a NifB fusion polypeptide having MTP-FAγ51 and HA fused at the N-terminus. The constructs were designed to enable purification of the AnfK polypeptide product using the Twin-Strep epitope and to test the possibility of co-purification of other Anf or Nif proteins.

Protein extraction and processing from the co-infiltrated plant samples were conducted under anaerobic conditions. The protein extract was passed through a StrepTactin XT affinity column and then eluted. Samples collected along the polypeptide purification process were analysed by SDS-PAGE and Western blotting with the HA and Strep-tactin antibodies for detection.

The Western blot probed with the Strep-tactin antibody showed the presence of processed AnfK fusion polypeptide in each of the total, input, pellet and eluate fractions, with bands at lower molecular weights potentially representing smaller AnfK-derived products, which were likely produced by post-extraction degradation by protease contamination. The purified AnfK fusion polypeptide was greatly concentrated in the eluate fraction compared to the input fraction as shown by the intensity of the AnfK band on the blot. When the Western blot was re-probed with the HA antibody, all of the encoded Anf and Nif fusion polypeptides were detected within the input sample, although the band for AnfG was visible only after exposure of the blot for 20 min rather than 1 min. Significantly, the HA antibody also showed the presence of the processed AnfD polypeptide within the eluate sample. The presence of AnfD and AnfK within the eluate sample indicated that when the AnfK fusion polypeptide was purified, the MPP-processed AnfD polypeptide was co-purified, indicating protein-protein interaction of these two fusion polypeptides.

### Homocitrate Production in the Infiltrated Plant Cells

As described in Example 15, the plant codon optimised A. vinelandii NifV fusion polypeptide (AvNifV) exhibited homocitrate synthase activity when expressed individually from the genetic construct SN254. The leaf samples infiltrated with either SL42 or SL43, or both vectors in combination, were assayed for the presence of homocitrate using the GC-MS/MS method as described in Example 15. Homocitrate was detected in the samples infiltrated with SL43, either alone or in combination with SL42, but not with SL42 alone. This was consistent with the presence of the AvNifV gene on SL43.

### Further Constructs for Combinations of Anf and NU′ Genes

As shown in Example 20, a fusion polypeptide with mitochondrial targeting that had joined AnfD with AnfK through an oligopeptide linker was expressed, efficiently processed, and was observed to be predominantly present in the soluble protein fraction after introduction of the genetic construct into plant cells. Therefore, a genetic construct was made which substituted the AnfD and AnfK genes on SL43 with a hybrid gene that encoded a MTP-CoxIV::TwinStrep::AnfD::Linker26(HA)AnfK fusion polypeptide (SEQ ID NO:203). This new vector was designated SL48.

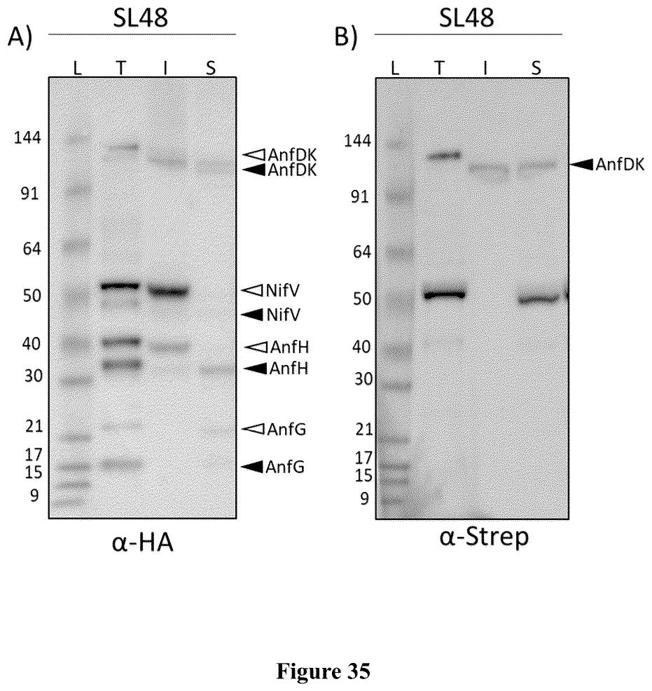

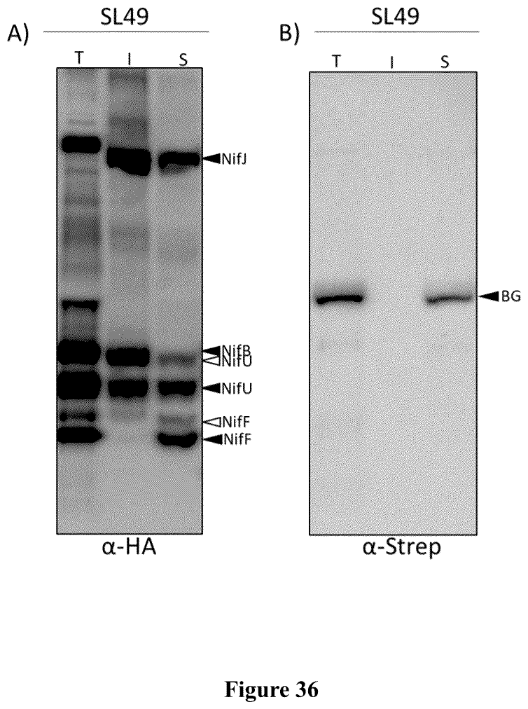

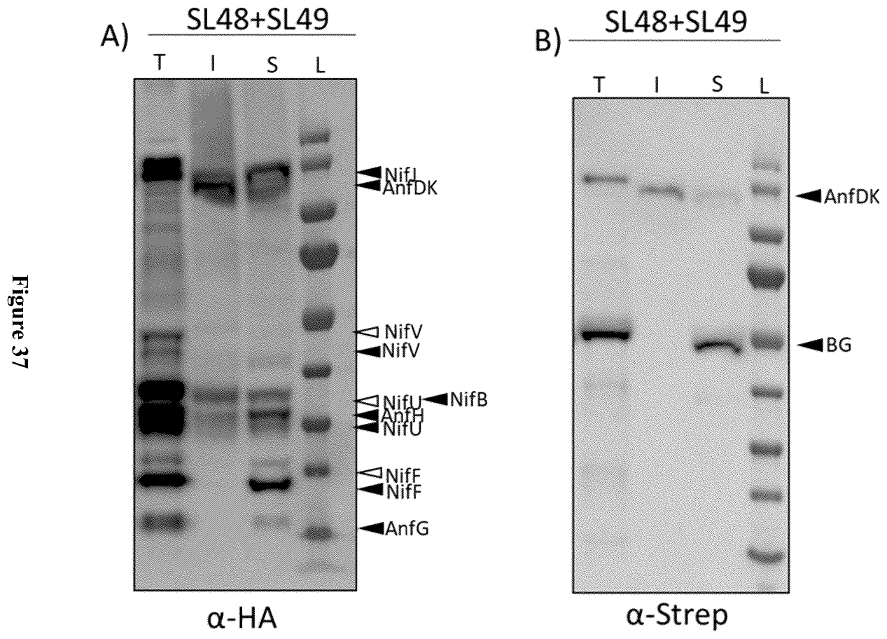

When SL48 and SL49 were introduced separately into N. benthamiana leaves, all of the encoded polypeptides were observed by Western blot analysis to be present in the soluble protein extracts to at least some extent (FIGS. 35 and 36). When the combination of SL48 and SL49 was introduced into N. benthamiana leaves, all eight of the encoded polypeptides were observed by Western blot analysis to be present in the soluble protein extracts, including the processed scar::TwinStrep::AvAnfD Linker26(HA)::AvAnfK fusion polypeptide (labelled as AnfDK in FIG. 37).

Another construct SL78 (Table 22) was made which was the same as SL49 except with addition of a fifth gene, encoding MTP-FAγ51::NifS::HA. SL48 and SL78 were infiltrated into N. benthamiana leaves, separately or in combination. Western blots of total, soluble and insoluble protein fractions showed the presence of all of the encoded fusion polypeptides in the soluble fraction as well as the total protein sample. That is, all 9 fusion polypeptides encoded by the combination of the two vectors could be detected, including the MPP-processed scar9::TwinStrep::AvAnfD::Linker26(HA)::AvAnfK fusion polypeptide from SL48. Therefore, all ten of the Anf and Nif proteins reported to be required as a minimum set to constitute Fe-nitrogenase in the bacterium E. coli (Yang et al., 2014) were produced in the plant cells, targeted to the mitochondria and present at least partially in soluble form.

Homocitrate production was detected in the infiltrated cells which had received SL48, indicating that the AvNifV gene on SL48 produced an active homocitrate synthase.

Protein Purification from Plant Cells Producing Anf and Nif Fusion Polypeptides.

Since the processed polypeptide encoded by the MTP-CoxIV::TwinStrep::AnfD::Linker26(HA)::AnfK gene on SL48 had a TwinStrep epitope translationally fused after the MTP sequence at its N-terminal end, the StrepTactinXT column purification method could be used to purify this fusion polypeptide from N. benthamiana cells that had been infiltrated with SL48 and SL49. Purification was carried out using the methods described above, and the purified scar::TwinStrep::AnfD::Linker26(HA)::AnfK polypeptide was concentrated using the method as described in Example 14.

The solution containing the purified polypeptide was seen to have a small amount of brown colour at the base of the sample. The inventors considered that this colour was due to the presence of Fe—S clusters bound to the scar::TwinStrep::AnfD::Linker26(HA)::AnfK polypeptide, indicating activity of at least the NifS, NifU and AnfH fusion polypeptides in the plant cells in providing the Fe—S clusters to the AnfD-linker-AnfK fusion polypeptide. This will be confirmed by measurement of the Fe²⁺ and S content in the isolated polypeptide using, for example, inductively coupled plasma mass spectrometry (TCPMS). Electroparamagnetic resonance (EPR) measurements are expected to detect specific wavelength shifts indicating the presence and structure of the Fe—S clusters bound to the polypeptide.

Increases in the amount of bound Fe—S clusters are expected by the addition of another gene to the Anf+Nif gene combinations described above, namely a gene encoding a ferredoxin such as FdxN from A. vinelandii or other nitrogen fixing organism (Example 22).

### Example 22. Expression of FdxN in Plant Cells with Mitochondrial Targeting

### Introduction

A FdxN gene is important for optimal function of nitrogenase in many diazotrophs, for example in A. vinelandii (Jimenez-Vicente et al., 2014; Buren et al., 2019). The genome of A. vinelandii strain CA (Setubal et al., 2009; www.ncbi.nlm.nih.gov/nuccore/NC_021149.1) has 16 ferredoxin-like genes including FdxN which belong to a class of 2x[4Fe-4S] cluster ferredoxins (Jimenez-Vicente et al., 2014). This class of ferredoxins contains two conserved motifs, Cys-X2-Cys-X2-Cys-X3-Cys and Cys-X2-Cys-X7˜9-Cys-X3-Cys-X3˜5-Cys, which are conserved in A. vinelandii FdxN except for the last Cys residue in the second motif (Matsubara and Saeki, 1992). FdxN genes functioning for nitrogenase in bacteria are often but not always found as part of an operon transcribed with other genes involved in nitrogenase, including Nif genes. For example, FdxN in A. vinelandii is part of a single operon containing NifB, FdxN, NifO-NifQ, RhdN and Grx5^{nif} protein coding regions. FdxN was transcribed at about the same level as NifB under diazotrophic growth conditions (Rodriguez-Quinones et al., 1993). When the nitrogenase enzymes were expressed in a ΔFdxN deletion mutant of A. vinelandii, a 5-fold reduction in NifB-co synthesis and consequently in nitrogenase activity was observed. The FdxN gene from A. vinelandii therefore encodes a ferredoxin protein which is involved in the synthesis of NifB-co that is required for all three of the Mo-, V- and Fe-nitrogenases. Deletion of FdxN also reduced the growth rate of A. vinelandii under diazotrophic conditions to about 50% of wild-type, indicating that the complete absence of FdxN was tolerated for growth and nitrogenase activity but needed for optimal growth and nitrogenase activity. FdxN in A. vinelandii is thought to act either as a ferredoxin in donating electrons to the NifB protein during the production of NifB-co or as an intermediate carrier of [4Fe-4S] to NifB, or both (Buren et al., 2019).

In contrast, FdxN in Rhizobium meliloti was demonstrated to be necessary for symbiotic nitrogen fixation, since fdxN mutants were unable to fix nitrogen. The function was restored by introducing a plasmid encoding FdxN (Klipp et al., 1988). Purified R. meliloti FdxN polypeptide was able to mediate electron transport to Rhodobacter capsulatus nitrogenase in vitro (Riedel et al., 1995). However, this absolute requirement for FdxN in R. meliloti was not reflected in many other diazotrophs such as R. capsulatus.

Different again, in K. oxytoca a flavodoxin (NifF) and a pyruvate:flavodoxin oxidoreductase (NifJ) mediate electron transfer from pyruvate to nitrogenase, not FdxN (Shah et al., 1983). Consistent with this, a K. oxytoca Nif gene cluster that produced functional nitrogenase when transferred to E. coli had the NifJHDKTYENXUSVWZMFLABQ genes but did not include a FdxN or equivalent gene (Smanski et al., 2014; Yang et al., 2013; Temme et al., 2012). The synthetic vector pMIT v2.1 expressed functional nitrogenase in E. coli without including a FdxN gene, although endogenous ferredoxins in E. coli might have provided such function. Proteins other than ferredoxin might also have substituted for FdxN function in E. coli, for example flavodoxins. Nitrogenase in diazotrophic bacteria commonly makes use of one or more flavodoxin proteins such as NifF and Nif7 as an electron donor, so NifF might have provided the function. In another study, Yang et al. (2017) replaced K. oxytoca NifF of the nitrogenase vector pKU7017 with Chlamydomonas or plant plastid ferredoxins from Arabidopsis, corn, rice and corn, all of which reduced acetylene at a rate of between 50-100% compared to control with NifF, showing that these ferredoxins could substitute for NifF at least for the function of electron donation to the NifH and NifD-NifK nitrogenase proteins. The vector pKU7017 did not include a K. oxytoca ferredoxin gene but does have a NifF gene, so the NifF protein or an endogenous E. coli ferredoxin might have provided electrons to NifH/NifD-NifK or to NifB for the formation of NifB-co, or both. In contrast, Yates 1972 found purified A. chromococcum flavodoxin, but not ferredoxin, could donate electrons to mature dinitrogenase. Jimenez-Vincente et al. (2014) confirmed the lack of electron donation of FdxN to NifD-NifK. The function of FdxN protein and its requirement for nitrogenase function is therefore not clear for different bacteria, let alone for nitrogenase when expressed in plants and targeted to mitochondria.

The structure and diversity of ferredoxins and related proteins has been reviewed by Matsubara and Saeki (1992).

### Phylogenetic Analysis of FdxN Polypeptides

A search of the NCBI non-redundant protein database using the A. vinelandii FdxN (SEQ ID NO:232) returned a hit to the protein family PRK13795 (hypothetical protein, provisional), which was the only member of the superfamily c136298. The 627 amino acid sequences in PRK13795 however coded for enzymes related to phosphoadenosine phosphosulfate reductase found in Archaea which were 400-800 amino acids in length and contained a [4Fe-4S] binding site, but no ferredoxin-like proteins. The protein information for FdxN from A. vinelandii strains DJ (Accession No. AC081189.1) and CA (WP_012703542.1) was annotated as belonging to family pfam12838. The region name of this domain was called “Fer4_7 4 Fe-4S dicluster domain” and pfam12838 was the only family member of the superfamily c138378. The description of pfam12838 was “Superfamily includes proteins containing domains which bind to iron-sulfur clusters”. Members include bacterial ferredoxins, various dehydrogenases, and various reductases. The structure of the domain was an alpha-beta sandwich and the domain contained two Fe₄S₄ clusters. There were 206 representative amino acid sequences listed in protein family pfam12838, of which 26 amino acid sequences were shorter than 160aa, used as a size cutoff since the longest sequence of the 16 A. vinelandii sequences annotated as a ferredoxin was 156 residues. The 26 amino acid sequences in pfam12838 of 93-156 amino acids in length were aligned using NCBI Global alignment (blast.ncbi.nlm.nih.gov/Blast) and the percentage identity to SEQ ID NO:232 (WP_012703542.1) determined. The percentage identity of the 26 sequences to SEQ ID NO:232 ranged between 10-22%, showing the diversity of FdxN sequences. The 26 sequences used in this analysis were from Accession Nos: Q8KG02_CHLTE, Q3ATN2_CHLCH, Q8KG03_CHLTE, Q9X2D5_THEMA, Q2JP81_SYNJB, Q9I1H8_PSEAE, Q01ZR2_SOLUS, ESU39497, WP_043013856, WP_012106131, WP_018723072, EKY12520, WP_012422852, ABG77170.1, EEX22670, WP_015853105, WP_012455913, WP_020095796, WP_012235387, WP_011973256, WP_015758977, WP_012302957, WP_012301895, WP_036081271, WP_004845399 and Q39V82_GEOMG.

### Single Gene Constructs to Express FdxN Fusion Polypeptides in Plant Cells

The inventors sought to express a gene encoding an A. vinelandii FdxN fusion polypeptide in plant cells, aiming for mitochondrial localisation of the FdxN gene product, as follows. Two genetic constructs (SN291, SN292) were first tested which expressed a MTP-FdxN fusion polypeptide on its own. Subsequent experiments combined the FdxN gene in a 5 gene construct together with genes encoding AnfD-Linker(HA)-AnfK, AnfH, AnfG and NifV fusion polypeptides, with the 5 genes on one T-DNA vector and each fusion polypeptide having an MTP sequence for mitochondrial targeting. A further experiment included a co-expression experiment with two 5 gene constructs, namely the vector encoding AnfD-Linker(HA)-AnfK, AnfH, AnfG, NifV and FdxN fusion polypeptides into one T-DNA vector, designated SL50, and SL49 (Example 21).

The two genetic constructs SN291 and SN292 were designed and made to express the FdxN fusion polypeptides (SEQ ID NO:233, SEQ ID NO:234) on its own in plant cells such as N. benthamiana leaf cells, and two control constructs SN299 and SN300 (Table 23). The synthetic genes were each under the control of the strong CaMV 35S promoter and a nos 3′ polyadenylation region/transcriptional terminator which flanked the protein coding region. The FdxN amino acid sequence from A. vinelandii (SEQ ID NO:232) with an added Ala residue at the C-terminus was used to design the nucleotide sequence of the protein coding region in each construct, with codon-optimisation for expression in plant cells. For mitochondrial localisation, SN291 encoded a fusion polypeptide having the MTP-FAγ51 fused to the N-terminus and a C-terminal HA-epitope for detection of the polypeptide by Western blotting with the anti-HA antibody. The HA epitope was fused translationally either C-terminally (SN291) or between the MTP and FdxN sequence (SN292). One control construct (SN300) encoded a polypeptide which lacked the MTP sequence and therefore expressed a smaller, cytoplasmically targeted polypeptide that provided a molecular weight comparator on the Western blots for an MPP-processed polypeptide from the MTP-FdxN polypeptides (processed FdxN), with the qualification that the MPP-processed polypeptide in each case included a “scar sequence” of about 9 amino acids and so the sizes were close but not identical. The second control construct (SN299) encoded a fusion polypeptide which had 13 amino acids in the MTP sequence substituted with alanines (Allen et al., 2017), designed to prevent processing by MPP. These second control polypeptides therefore provided a molecular weight comparator for the unprocessed polypeptide from the corresponding MTP-FdxN constructs. The alanine-mutated MTP sequence was designated mFAγ51. When protein extracts from the infiltrated plant tissues were analysed, the sample from each MTP-FdxN construct and its two corresponding control constructs were loaded onto adjacent lanes for gel electrophoresis, so allowing for the best detection of processing of the MTP-FdxN polypeptide.

TABLE 23
Single and multi-gene genetic constructs encoding FdxN and
components of Fe-nitrogenase for expression in plant cells.

| | | Predicted molecular | | SEQ ID |
|---|---|---|---|---|
| | | weight (kDa) | | NO for |
| Plasmid | Expressed polypeptide | Unprocessed | Processed | Unprocessed |
| SN291 | MTP-FAγ51::FdxN::HA | 16.6 | 11.9 | 233 |
| SN292 | MTP-FAγ51::HA::FdxN | 16.4 | 11.9 | 234 |
| SN299 | mFAγ51::HA::FdxN | 15.8 | NA | 235 |
| SN300 | HA::FdxN | NA | 10.8 | 236 |
| SL50 | MTP-FAγ51::HA::AnfG | 22.2 | 17.7 | 186 |
| | MTP-CoxIV::TwinStrep::AnfD::Linker26(HA)::AnfK | 118 | 115 | 203 |
| | MTP-FAγ51::HA::AnfH | 36.9 | 32.4 | 185 |
| | MTP-FAγ51::HA::NifV | 48.4 | 43.8 | 237 |
| | MTP-FAγ51::HA::FdxN | 16.4 | 11.9 | 233 |
| SL54 | MTP-FAγ51::NifF::HA | 26.1 | 21 | 137 |
| | MTP-FAγ51::NifJ::HA | 135 | 130 | 138 |
| | MTP-FAγ51::NifS::HA | 50.3 | 45 | 124 |
| | MTP-FAγ51::NifU::HA | 36 | 31 | 125 |
| | MTP-FAγ51::KoNifB::HA | 58 | 53 | 147 |

### Production of the Fusion Polypeptides in Plant Cells

Cultures of A. tumefaciens containing SN291 were infiltrated into 5-week-old N. benthamiana leaves as described in Example 1. Four to five days post infiltration, leaf samples were harvested. Total, soluble and insoluble protein fractions were extracted, as follows. For testing the solubility of plant-expressed polypeptides, the leaf tissue was ground in ice-cold extraction buffer (100 mM Tris pH 8.0, 150 mM NaCl, 0.25 M mannitol, 5% (v/v) glycerol, 1% (v/v) Tween 20, 1% (w/v) PVP, freshly-added 2 mM TCEP, 0.2 mM PMSF and 10 μM leupeptin) and transferred to a microfuge tube. The sample was centrifuged at 20,000×g for 5 min to divide the sample into soluble (supernatant) and insoluble (pellet) fractions. The supernatant was transferred to a fresh microfuge tube and centrifuged again at 20,000×g for 5 min to remove any remaining insoluble material. The insoluble fraction was washed by resuspension of the pellet in 3004 extraction buffer, with dispersal by repeated strokes of the pipette, and centrifuged at 20,000×g for 5 min, discarding the supernatant. This washing step was repeated twice more, removing any remaining soluble protein from the insoluble fraction. Samples were then analysed by SDS-PAGE and Western blotting using anti-HA antibody. The anti-HA antibody (Monoclonal Anti-HA, Sigma) was used at 1:5000 dilution.

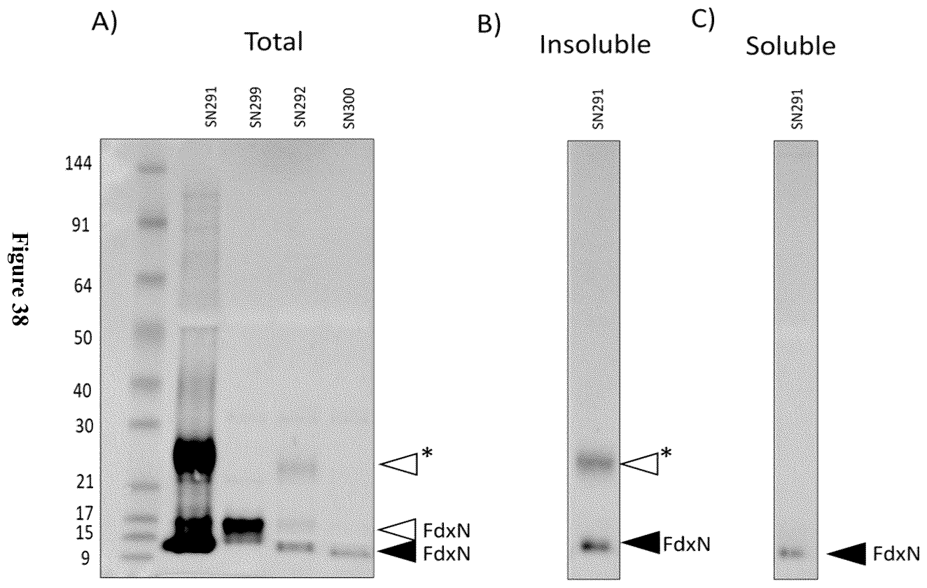

The Western blot analysis for SN291 (FIG. 38) showed that the FdxN polypeptide was readily detected in the total protein fraction with the HA-antibody, showing a faint polypeptide present in both the soluble and insoluble protein fractions, requiring longer exposures in the Western procedure to be visible, indicating the AvFdxN fusion polypeptide was partially soluble. The FdxN fusion polypeptide appeared to be partially processed by MPP, with both processed and unprocessed forms, indicating inefficient cleavage by MPP. The bands in the adjacent lanes for the control polypeptides in each case confirmed that these bands were for processed and unprocessed polypeptides.

Cultures of A. tumefaciens containing SN292 were infiltrated into 5-week-old N. benthamiana leaves as described in Example 1. Four to five days post infiltration, leaf samples were harvested. Total, soluble and insoluble protein fractions were extracted, using the same method as for SN291. The Western blot analysis for SN292 showed that the FdxN polypeptide was readily detected in the total protein fraction with the HA-antibody, indicating that the position of the HA epitope in the fusion polypeptide, either C-terminal or towards the N-terminus, did not affect the level of expression of the polypeptide. Again, the FdxN fusion polypeptide appeared to be partially processed by MPP, with the majority of the protein at the correct size for the processed forms. The bands in the adjacent lanes for the control polypeptides in each case confirmed that these bands were for processed polypeptides.

### Production of Combinations of Fusion Polypeptides Including FdxN in Plant Cells

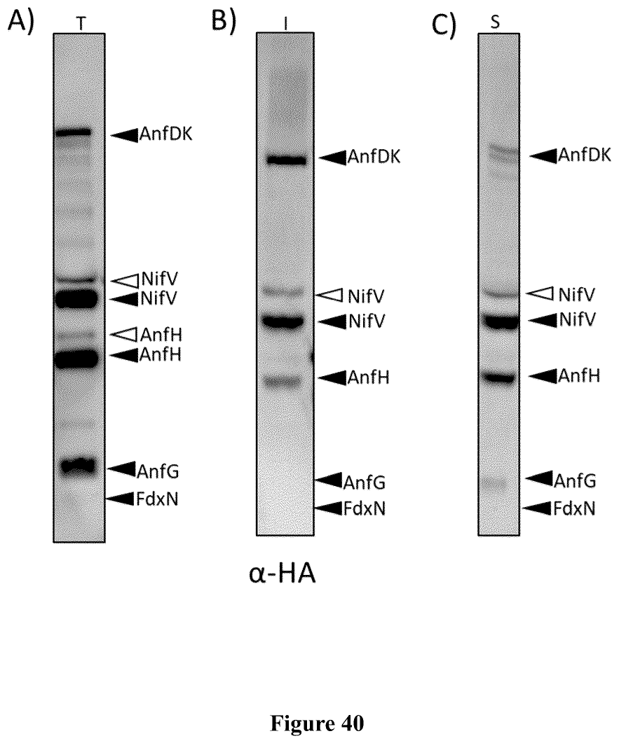

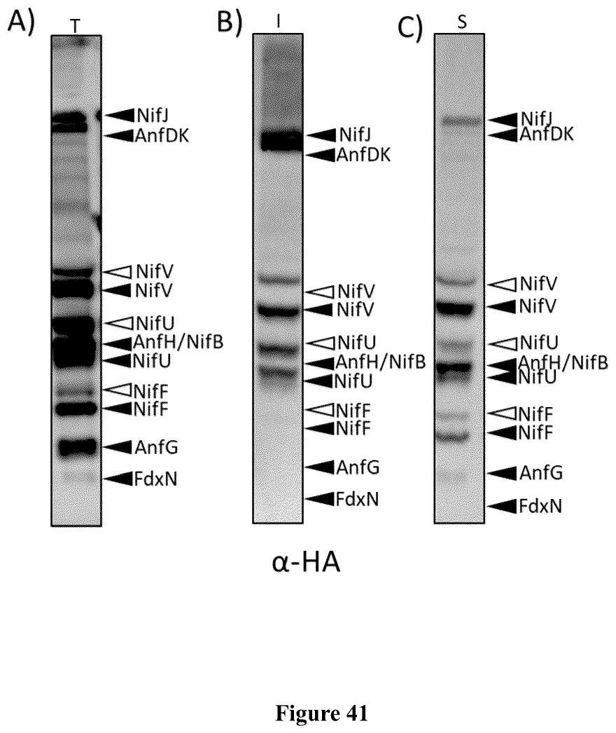

A new genetic construct was designed and made using the GoldenGate synthesis method, designated SL50 (Table 22), and tested separately (FIG. 40) and in combination with SL49 or SL54 (FIG. 41). One gene on SL50 encoded the MTP-CoxIV::TwinStrep::AnfD::Linker26(HA)::AnfK fusion polypeptide and the other four genes encoded AnfH, AnfG, NifV and FdxN fusion polypeptides, with each having the MTP-FAγ51 sequence followed by a HA epitope translationally fused at the N-terminus of the polypeptides. The genetic constructs SL49 and SL50 were introduced into N. benthamiana cells separately and protein expression analysed by Western blot. All five of the encoded fusion polypeptides from SL50 were detected with the appropriate antibodies, each showing polypeptide bands present in the soluble protein fraction, with exception of the FdxN polypeptide which was not visible within either the soluble or insoluble fractions (FIG. 40). Importantly, the processed AnfD-linker-AnfK, NifV and AnfH fusion polypeptides were all observed in the soluble fraction as well as the insoluble fraction, so all three were at least partially soluble. The AnfG, AnfH and NifV polypeptides all appeared to be partially cleaved, each showing bands for the processed and unprocessed forms. The AnfD-linker-AnfK polypeptide appeared to be efficiently processed. The FdxN polypeptide was only visible after a long exposure time and could only be seen at the processed size within the total protein.

Next, the Agrobacterium cultures containing SL50 and SL49 were mixed and the mixture infiltrated into N. benthamiana leaves as before. This experiment therefore introduced 3 Anf genes encoding the AnfH and AnfG polypeptides and the fused AnfD-linker-AnfK polypeptide, 5 of the Nif genes, encoding NifF, NifJ, NifU, NifB and NifV polypeptides, and the FdxN gene, i.e. 9 genes in combination. The surprising result observed in the Western blot (FIG. 41) was that all 9 polypeptides were readily detected. Several of the polypeptides were visible with 2 bands, with the upper band representing unprocessed polypeptide and the lower band representing the cleaved polypeptide upon mitochondrial import. The unprocessed polypeptide band was visible for the NifV, NifU and NifF fusion polypeptides as well as a band present at the predicted size for cleaved polypeptides. All of the polypeptides with the exception of the FdxN polypeptide were present within the soluble fraction, which was not visible within either the soluble or insoluble fractions due to its low level of accumulation.

Another genetic construct was designed and made using the GoldenGate synthesis method, designated SL54 (Table 23), and tested separately and in combination with SL50. SL54 had a gene encoding a MTP-FAγ51::NifB::HA fusion polypeptide (SEQ ID NO:147) which used the sequence from K. oxytoca, but otherwise SL54 was identical to SL42 (Table 22) for expression of NifS, NifU, NifJ and NifF fusion polypeptides. This experiment also tested whether the NifB fusion polypeptide based on the K. oxytoca sequence, previously shown to be mostly insoluble when expressed on its own, might be improved in its solubility when expressed in combination with the other polypeptides.

SL50 and SL54 were first of all introduced separately into N. benthamiana leaves and soluble and insoluble protein fractions prepared and analysed by Western blot analysis as well as the total protein fractions. All of the encoded polypeptides were observed to be present in the soluble protein extracts to at least some extent as well as in the total protein fractions, with the exceptions of the NifB and FdxN polypeptides which were either not visible or obscured by other protein bands of a similar size. The least intense polypeptide was the FdxN polypeptide which was only visible in the total protein sample after a longer exposure.

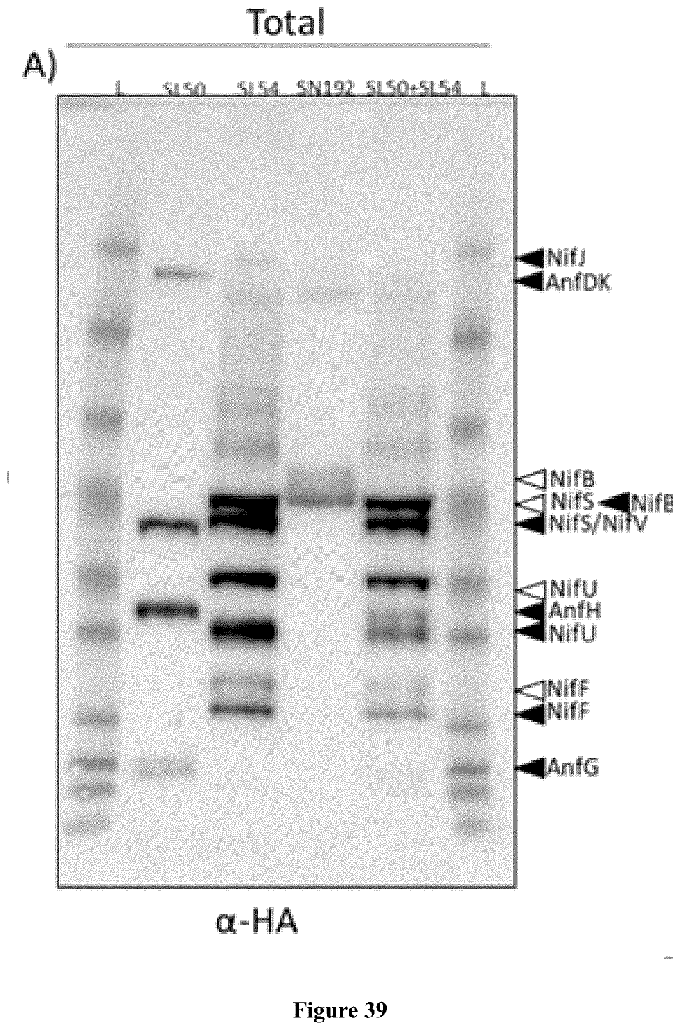

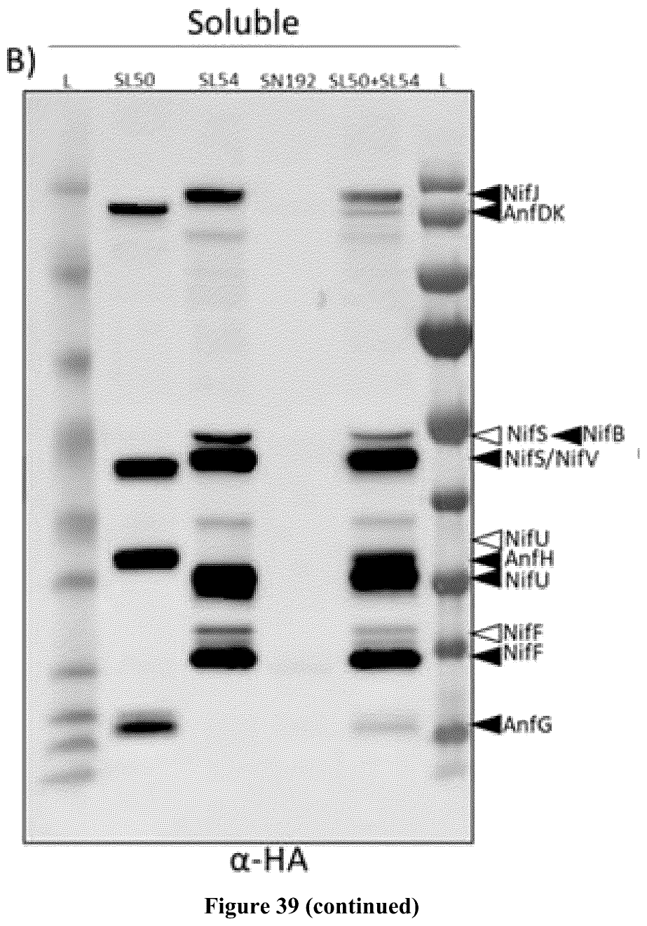

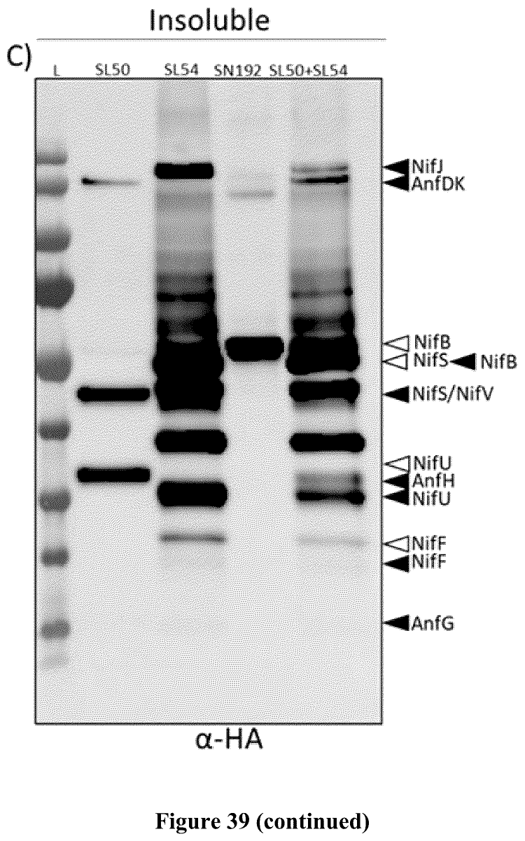

SL50 and SL54 were also introduced together into N. benthamiana leaves. The Antes, AnfG, NifV, NifJ, NifS, NifU and NifF polypeptides as well as the processed AnfD-linker-AnfK polypeptide were all observed by the Western blot analysis to be present in the soluble protein extracts to at least some extent. Again, the FdxN polypeptide was not visible in either the soluble or insoluble fractions due to its low level of accumulation. The presence of the NifB polypeptide within the soluble fraction could not be confirmed due to its size which coincided in the SDS-polyacrylamide gels with the unprocessed NifS polypeptide (FIG. 39).

As a size and solubility control, a single gene vector (SN192) encoding the K. oxytoca NifB polypeptide was separately infiltrated into N. benthamiana leaves. When the Western blot was probed with the HA antibody, the NifB polypeptide was visible as both the unprocessed and processed forms and was visible within the total protein and insoluble fractions, with no NifB visible within the soluble fraction (FIG. 39).

In further experiments, the TwinStrep-AnfD-linker-AnfK polypeptide expressed from SL50 will be purified. In further experiments, combinations of SL50 with variants of SL54 will be tested, the variants having NifB polypeptides originating from organisms other than K. oxytoca.

Generation of Plants Stably Transformed with Anf and Nif Genes

Each set of genes in SL49 and SL50 were transferred separately to a binary vector having a selectable marker gene. The resultant constructs were used to generate transformed A. thaliana plants by the floral dip method. After initial selection with kanamycin, nine T1 transformants were obtained for SL49 and two T1 transformants obtained for SL50. These transgenic plants are expected to express all of the encoded polypeptides, incorporate Fe—S clusters such as P-cluster into the AnfD-linker-AnfK and Antes polypeptides and produce homocitrate in increased amounts relative to corresponding wild-type plants or plants lacking the NifV gene. The constructs are also used to transform other plants, for example tobacco (N. tabacum) and N. benthamiana.

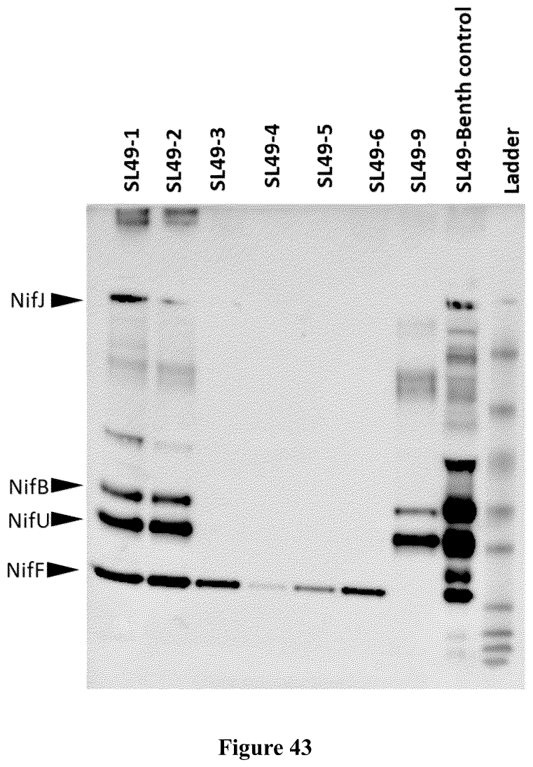

When leaf samples of the 9 plants transformed with the T-DNA from SL49 were analysed by Western blot, 2 of the plants (SL49-1 and SL49-2) were observed to be producing all four of the encoded Nif fusion polypeptides: NifB, NifF, NifJ and NifU (FIG. 43). Four of the plants (SL49-3 to 6) were expressing only the NifF polypeptide and were assumed to represent plants in which only part of the T-DNA had been integrated into the genome, namely encoding NifF, or if the other Nif genes had been incorporated they were not expressed. One plant (SL49-9) was expressing the NifB and NifU polypeptides, possibly NifJ as well, but not the NifF polypeptide, and again were assumed to have a partial T-DNA integrated. In each of the plants expressing Nif polypeptides, the fusion polypeptides appeared to be efficiently processed by MPP to produce the scar::Nif::HA or scar::HA::Nif polypeptides, based on comparison with the same polypeptides expressed transiently in N. benthamiana. It was concluded that the polypeptides produced in the stably transformed plants were processed by MPP more efficiently, indeed even completely, than when expressed transiently in the N. benthamiana leaves.

The DNA region including the set of genes in SL79, namely genes for AnfD-linker-AnfK, AnfH, AnfG, NifV and FdxN fusion polypeptides, all having a HA or TS epitope (Table 22) and in SL29 encoding the MTP-FAγ51::HA::AvAnfD fusion polypeptide were separately transferred to a binary vector having a selectable marker gene for kanamycin resistance. The resultant constructs were used to generate transformed A. thaliana plants by a standard floral dip method. Seeds (T1) were obtained from the treated plants and plated onto selective media to select transgenic (T1) plants. After the selection with the antibiotic kanamycin, five T1 transformants were obtained for SL29 and two T1 transformants obtained for SL79. Plant samples were collected for measurement of homocitrate production. These transgenic plants were expected to express all of the encoded polypeptides, incorporate Fe—S clusters such as P-cluster into the AnfH polypeptides as well as the AnfD-AnfK protein complex or the AnfD-linker-AnfK polypeptides and produce homocitrate in increased amounts relative to corresponding wild-type plants or plants lacking the NifV gene.

Of the 5 plants transgenic for the T-DNA from SL29, four were observed by Western blotting to be producing the scar::HA::AnfD polypeptide. The 2 plants transgenic for the T-DNA from SL79, namely SL79-1 and SL79-2, were observed by Western blotting to be producing the scar-AnfD-linker-AnfK, scar-AnfH, scar-AnfG, scar-NifV and scar-FdxN fusion polypeptides.

To produce stably transformed plants expressing nine of the Nif/Anf/Fdx fusion polypeptides, the SL79-1 plants are crossed with SL49-1 plants to produce F1 and F2 plants containing both T-DNAs. To produce plants stably transformed with an additional NifS gene, the experiment is repeated with SL78 rather than SL49, so producing plants expressing the combination of nine Nif/Anf genes as the expected minimal set for expressing functional Fe-nitrogenase as well as FdxN.

### Example 23. Analysis of Anf Polypeptides

As described herein, an AnfH polypeptide is a NifH polypeptide which is a member of the nitrogenase conserved superfamily c125403 containing the PRK13233 conserved domain and having at least 69% amino acid sequence identity to the Azotobacter vinelandii AnfH polypeptide (SEQ ID NO:218) when measured along the full length of 275 amino acid residues of SEQ ID NO:218. The inventors analysed AnfH polypeptide sequences present in databases and aligned these and compared them to a representative molybdenum-type NifH.

Databases were searched for AnfH amino acid sequences. These were identified as having the PRK13233 conserved domain and at least 69% identity to SEQ ID NO:218. This identified 314 such sequences. These were aligned with NCBI COBALT and a consensus sequence developed which had 300 residue positions including gaps. This consensus sequence was 89% identical to SEQ ID NO:218. The aligned AnfH amino acid sequences remarkably had 137 amino acids of the 300 positions that were identical in all of the 314 naturally occurring AnfH polypeptides and many other amino acids that were conserved in many of the AnfH polypeptides. Since the 137 conserved amino acids within the PRK13233 domain spanned most of the AnfH sequences, it was concluded that the PRK13233 domain covered most of the AnfH sequences and that PRK13233 was indicative of a family of sequences, not of one particular sequence. The 137 conserved amino acids included the sequence motifs YGKGGIGKSTTXQNT (motif I, SEQ ID NO:225), IHGCDPKAD (motif II, SEQ ID NO:226), CVESGGPEPGVGCAGRG (motif III, SEQ ID NO:227), DVLGDVVCGGFAMP (motif IV, SEQ ID NO:228), VASGEMMAXYAANNI (motif V, SEQ ID NO:229), QSGVR (motif VI, SEQ ID NO:230) and CNSRXVD (motif VII, SEQ ID NO:231), where X represents any amino acid. All of the motifs I-VII were present in all 314 AnfH sequences analysed.

The 137 amino acids that were fully conserved were as follows, with the number referring to the amino acid position in SEQ ID NO:218 and the letter to the amino acid at that position: 3R, 4K, 6A, 8Y, 9G, 10K, 11G, 12G, 131, 14G, 15K, 16S, 17T, 18T, 20Q, 21N, 22T, 25A, 361, 37H, 38G, 39C, 40D, 41P, 42K, 43A, 44D, 46T, 47R, 50L, 52G, 55Q, 60D, 63R, 75V, 79G, 85C, 86V, 87E, 88S, 89G, 90G, 91P, 92E, 93P, 94G, 95V, 96G, 97C, 98A, 99G, 100R, 101G, 1031, 104T, 1061, 108L, 109M, 110E, 115Y, 119L, 120D, 125D, 126V, 127L, 128G, 129D, 130V, 131V, 132C, 133G, 134G, 135F, 136A, 137M, 138P, 140R, 142G, 143K, 144A, 146E, 148Y, 150V, 151A, 152S, 153G, 154E, 155M, 156M, 157A, 159Y, 160A, 161A, 162N, 163N, 1641, 167G, 170K, 172A, 174Q, 175S, 176G, 177V, 178R, 180G, 181G, 184C, 185N, 186S, 187R, 189V, 190D, 192E, 198E, 199F, 204G, 212P, 213R, 215N, 217V, 218Q, 220A, 221E, 222F, 227V, 236Q, 239E, 240Y, 243L, 2471, 250N, 254V, 2551, 256P, 258P, 265E, 272G. When aligned with the A. vinelandii NifH sequence (AvNifH; SEQ ID NO:224), 121 of the 137 fully conserved amino acids from AnfH sequences were also present in the corresponding positions of AvNifH. The 16 amino acids that were conserved in all of the AnfH sequences but not in AvNifH were: 4K, 22T, 37H, 52G, 60D, 63R, 108L, 109M, 142G, 151A, 174Q, 189V, 198E, 199F, 222F and 2471 with reference to SEQ ID NO:218. These 16 amino acids were therefore characteristic of AnfH relative to the molybdenum-type NifH sequence of AvNifH and can be used to distinguish AnfH polypeptides from other NifH sequences which do not have all 16 amino acids in common. AvNifH, KoNifH (SEQ ID NO:1) and other molybdenum type NifH sequences had motifs III and IV but did not have motifs I, II, V-VII, and therefore these motifs could also be used to distinguish the AnfH subset from other NifH polypeptides.

### Example 24. Co-Expression of Additional Nif Polypeptides Improves Abundance of NifD-NifK Complex and NifY

A mature and catalytically active Mo-nitrogenase includes two metallofactors, the P-cluster and the FeMo-co cluster. These metalloclusters are assembled in several steps in an order reported in Buren et al. (2019), based largely on studies with A. vinelandii nitrogenase. For the synthesis of the P-cluster, a NafH polypeptide interacts with a protein complex referred to as pre-apo-NifD-NifK and aids the placement of 2 separate [Fe₄—S4] clusters, donated from NifU, onto positions within the NifD and NifK polypeptides. The NafH-NifD-NifK interaction is then replaced by a NifW-NifD-NifK interaction. The NifW polypeptide is then displaced by a mature NifH and NifZ, and at this stage the [Fe₄—S4] clusters are condensed into the [Fe₈—S_{7] cluster on the interface of NifD and NifK, the so-called P-cluster, with elimination of one sulfur atom. The formation of the P-cluster converts pre-apo-NifD-NifK to one, perhaps two, ‘apo-NifD-NifK’ intermediates which bind to NafY (also referred to as a γ protein) and/or NifY. In the case of NafY, structural studies have shown that a N-terminal domain on NafY binds to apo-NifD-NifK and a C-terminal domain binds to binds to FeMo-co. FeMo-co is formed elsewhere on NifE-NifN and NifX is thought to be involved in shuttling the metallofactor between the proteins.}

This sequential assembly pathway and its putative protein interactions are based on studies of A. vinelandii nitrogenase and some of these steps are likely different or use different proteins in other organisms. For example, Klebsiella oxytoca does not have genes encoding NafH or NafY and its NifY is more similar to NafY than NifY in A. vinelandii. NifX in K. oxytoca was not needed for diazotrophy (Temme et al., 2012). Only functional FeProtein (NifH) is required for the formation of P-clusters in Klebsiella as deletion of the NifH gene disrupted P-cluster formation and diazotrophic growth. In contrast, deletion separately of genes encoding NafY, NifY, NifW or NifZ in A. vinelandii slowed but did not stop diazotrophic growth, indicating that these components were partially redundant or that a lack of a particular protein could be compensated by other factors in A. vinelandii.

The present inventors decided to test the effect on a NifD-NifK fusion polypeptide of co-expression of NifW, NifX, NifY and NifZ polypeptides in plant cells with mitochondrial targeting. To do this, a plant expression construct designated SL55 was made using Golden Gate cloning methods. SL55 had four Nif genes encoding KoNifW, KoNifX, KoNifY and KoNifZ fusion polypeptides, each based on the K. oxytoca sequence and having an N-terminal fusion to the MTP-FAγ51 sequence. Each polypeptide also had a HA epitope fused at the C-terminus for detection in Western blots. Components used for construction of SL55 were from SN340 (MTP-KoNifW-HA), SN144 (MTP-KoNifX-HA), SN145 (MTP-KoNifY-HA) and SN146 (MTP-KoNifZ-HA). Each individual gene was flanked by a 35S-promoter and a 3′ polyadenylation region/transcription terminator for expression in plant cells. The second genetic construct used in the co-infiltration experiment was SL47, encoding mitochondrially targeted MTP-FA751::KoNifDY100Q::1inker26(HA)::KoNifK, as encoded by SN159. This translational fusion had the NifD sequence based on the K. oxytoca sequence with Y100Q substitution within the NifD sequence. The constructs SL55 and SL47 were infiltrated either separately or together into N. benthamiana leaves and samples taken 4 or 5 days after infiltration for Western blot analysis. Proteins were extracted under aerobic conditions, resolved on a 4-20% gradient gel (SDS-PAGE) and probed with anti-HA antibody and HRP secondary antibody as before.

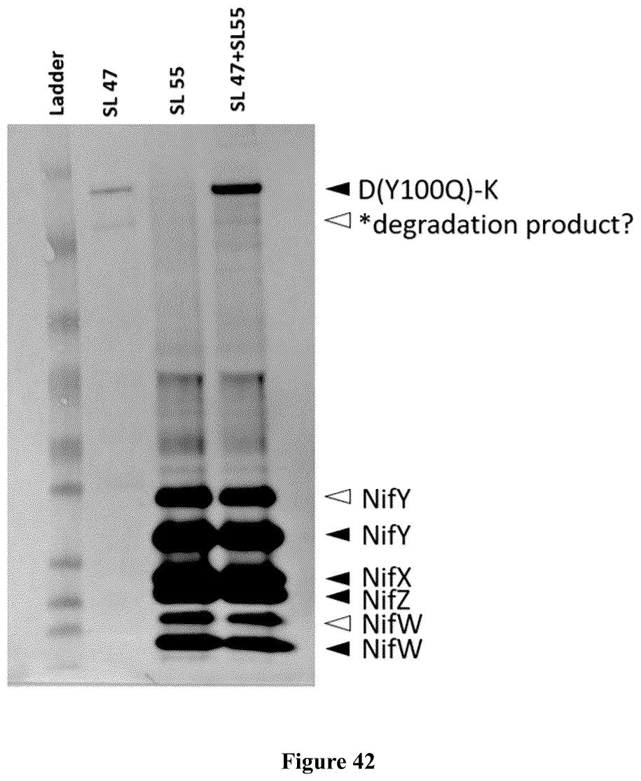

Leaves infiltrated with SL47 alone produced a relatively weak signal for a polypeptide of ˜110 kDa, expected for the size of the scar::NifD::linker26(HA)::NifK polypeptide (FIG. 42). Leaves expressing the four MTP-Nif fusion genes on SL55 either alone or co-infiltrated with SL47 produced strong signals for correctly MPP-processed NifW, NifX, NifY and NifZ polypeptides, as described in Examples 2 and 3. Surprisingly and significantly, the leaves co-infiltrated with SL55 and SL47 resulted in a much greater intensity of the band corresponding to correctly processed scar::NifD::linker26(HA)::NifK polypeptide (FIG. 42). It was also noted that a weaker band produced from SL55 at about 100 kDa, possibly arising from secondary degradation of scar::NifD::linker26(HA)::NifK polypeptide within the mitochondria, was less abundant when SL47 was co-infiltrated with SL55. This reduced abundance of the putative degradation product occurred despite the greater abundance of the correctly processed scar::NifD::linker26(HA)::NifK polypeptide.

Additionally, co-expression of a combination of single-gene vectors SN340, SN144, SN145 and SN146 resulted in greater intensity of the band for correctly processed NifY relative to expression from SN145 alone. This result suggested that the combination of SN340, SN144, and SN146 (NifW, NifX and NifZ fusion polypeptides) improved the expression and/or the stability of the NifY fusion polypeptide in the plant mitochondria. The inventors concluded that one or more than one or the combination of mitochondrially targeted NifW, NifX, NifY and NifZ improved the abundance of the translational fusion of NifD and NifK polypeptides. This experiment also showed that co-expression of NifW, NifX, and NifZ polypeptides improves the abundance of NifY in plant cells.

Another construct (SN229) was made encoding a similar NifD-NifK fusion polypeptide but including a Twin-strep epitope to provide for purification of the MPP-processed polypeptide from plant cells. SN229 was co-infiltrated into N. benthamiana leaves with SL55. Protein extracts are prepared and passed through a Strep-tactinXR column under aerobic or anaerobic conditions. The eluate from the column contains purified scar::TS::NifD::linker26(HA)::NifK polypeptide and is analysed for the presence of NifW and NifZ fusion polypeptide, one or both of which are expected to co-purify with the NifD-NifK protein.

The constructs SN299, SL55 and a third construct encoding separate NifH, NifM, NifS and NifU fusion polypeptides, all of them mitochondrially targeted by fusion with an MTP, are co-infiltrated into N. benthamiana leaves. Protein extracts are again prepared and passed through a Strep-tactinXR column under aerobic or anaerobic conditions. The resulting eluate is expected to contain purified scar::TS::NifD::linker26(HA)::NifK polypeptide which has properly formed P-cluster bound to it, available for receiving FeMo-co, i.e. an apo-NifD-NifK polypeptide. The level of P-cluster is measured using ICP-MS.

### Example 25. Cysteine Desulfurase Activity of NifS Expressed in Plant Cells and Targeted to Mitochondria

As described in Example 14, wild-type NifS polypeptide is a cysteine desulfurase which in diazotrophic bacteria generates the inorganic sulphide required for Fe—S cluster synthesis from cysteine, producing alanine as a byproduct after removal of the sulphur from the cysteine substrate. To test directly for this activity, the plant expression construct SN231, encoding MTP-FAγ51::NifS::TS, was infiltrated into N. benthamiana leaves and NifS fusion protein comprising the TwinStrep epitope purified using a StrepTactinXT column as described in Example 14. The elution buffer used to purify the protein was replaced with an assay buffer containing 25 mM Tris-HCl, pH 8.0, and 1 mM DTT by three rounds of passing the purified protein through a 4 mL Amicon Ultra kDa MWCO concentrator, then resuspending the concentrated protein in the assay buffer. The total dilution factor of the elution buffer by this method was approximately 1:5600. The final protein concentration in the assay buffer was estimated to be 0.46 mg/ml. The substrate cysteine was added to the purified protein at a final concentration of 0.5 mM and the reaction mixture incubated in the anaerobic hood at ambient temperature for 2.5 h, after which the reaction was stopped by adding methanol to a final concentration of 86% of the assay solution.

To measure conversion of cysteine and production of alanine in the reactions, GC tandem mass spectrometry (GC-MSMS) was used as follows. Each reaction mixture was dried in a vacuum concentrator prior to metabolite derivatization, which was carried out as follows. To each dried sample, 10 μl of 20 mg/ml methoxyamine hydrochloride in pyridine was added. The solutions were incubated at 37° C. for 90 min with vortexing at 15 min intervals, then 15 μl of a mixture of N,O-bis(trimethylsilyl)trifluoroacetamide and trimethylchlorosilane (BSTFA+TMCS) (99:1) was added and the solution incubated at 37° C. for 30 min with vortexing at 15 min intervals. 5 μl of alkane mix (n-dodecane, n-pentadecane, n-octadecane, n-eicosane, n-pentacosane, n-heptacosane, n-dotriacontane at 0.029% w/v each) was added as molecular weight markers and mixed. Each derivatization mix was left at ambient temperature for 60 min before GC-MSMS analysis.

The GC-MSMS analysis was conducted on a Shimadzu TQ8050 gas chromatography tandem mass spectrometer fitted with a DB-5 capillary column (30 m×0.25 mm ID×1 μm film thickness). One μl was injected at 1:10 split mode onto the column with the inlet heated to 280° C. and helium as carrier gas. The oven temperature was set to 100° C., held for 4 min, then increased to 320° C. at 10° C./minute, and held for 11 min. The mass spectrometer interface was heated to 280° C., ion source at 200° C. Masses between 45 and 600 were measured in full-scan mode. For multiple reaction monitoring (MRM) mode, the Shimadzu MRM library containing 467 compounds with target and qualifier ions between particular retention time windows, set for each metabolite derivative, was used for detection with the same GC and MS parameters. Multiple reaction monitoring (MRM) parameters for alanine 2TMS and cysteine 3TMS were as follows. Alanine 2TMS was measured by detection of two fragmentation patterns at retention index 1105: target ions m/z=116>73 at 15 volts, and reference ions m/z=190/147 at 9 volts. Cysteine 3TMS was measured by detection of two fragmentation patterns at retention index 1564: target ions m/z=220>73 at 24 volts, and reference ions m/z=218/73 at 24 volts.

The results of the assays are provided in Table 24. The reaction mixtures having water, assay buffer-only and assay buffer containing NifS without added cysteine did not have detectable amounts of cysteine. When cysteine was added as substrate to the assay containing NifS, the peak area of cysteine (measured as the cysteine 3TMS derivative) decreased by three-fold compared to when it was added to buffer-only, showing that the cysteine was being consumed. A low level of alanine 2TMS was detected in the reaction mixtures for buffer-only and buffer-only with added cysteine—this low level of alanine was considered to be a contaminant. In contrast, in the assay containing NifS the peak area of alanine increased by 18.5-fold. The consumption of cysteine and concomitant increase in alanine in the assay containing NifS and cysteine was definitive evidence that the NifS that was expressed, targeted to the mitochondrial matrix and processed in N. benthamiana leaf cells was functional as a cysteine desulphurase. It was noteworthy that the NifS polypeptide assayed was a processed fusion polypeptide that had a nine amino acid scar (scar9) plus two glycines at the N-terminus and a 28 amino acid TS tag plus two glycines at the C-terminus, yet it was able to convert cysteine to alanine.

TABLE 24
Peak areas of alanine 2TMS and cysteine 3TMS of water, assay buffer-
only, and assay containing NifS purified from N. benthamiana leaf,
with and without cysteine. +cys, with added cysteine.

| Compound Name | water | buffer | buffer + cys | NifS | NifS + cys |
|---|---|---|---|---|---|
| Cysteine 3TMS | 0 | 0 | 208920 | 0 | 70116 |
| Alanine 2TMS | 0 | 30564 | 30632 | 8870 | 164965 |

The present application claims priority from AU 2019903818 filed 10 Oct. 2019 and AU 2020900689 filed 5 Mar. 2020, the entire contents of all of which are incorporated herein by reference.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abe et al. (2000). Cell 100:551-560.
Abdullah et al. (1986). Biotechnology 4(12):1087.
Allen et al. (1994). Crit. Rev. Biotechmol. 14:225-249.
Allen et al. (1995). J. Biol. Chem. 270:26890-26896.
Allen et al. (2017). Front. Plant Sci., 8:287. doi: 10.3389/fpls.2017.00287
Arnold et al. (1988). Tree physiology, 4:291-300.
Altschul et al. (1997). Nucleic Acids Res. 25:3389-3402.
Balk and Pilon (2011). Trends Plant Sci 16:218-226.
Barker et al. (1983). Plant Molecular Biology, 2(6):335-350.
Becker et al. (2012). Trends in Biochemical Sciences 37:85-91.
Bevan et al. (1983). Nature 304:184-187.
Boison et al. (2006). Arch. Microbiol. 186:367-376.
Boyd et al. (2011) Geobiology 9:221-232
Boyd and Peters (2013). Front. Microbiol. 4:201. doi:10.3389/fmicb.2013.00201.
Brigle et al. (1987). J. Bacteria 169:1547-1553.
Bruce (2001). Biochimica et Biophysica Acta (BBA)—Molecular Cell Research 1541:2-21.
Buren et al. (2017a). Front Plant Sci 8:1567.
Buren et al. (2017b). ACS Synthetic Biology 6(6):1043-1055.
Buren and Rubio (2018) Fems Microbiol Lett 365:fnx274; doi: 10.1093/femsle/fnx274.
Buren et al. (2019). Proc. Nat. Acad. Sci. USA 116:25078-25086.
Buren et al. (2020). Chemical Reviews doi.org/10.1021/a cs.chemrev.9b00489.
Cannon et al. (1988) Nucleic Acids Res. 16:11379.
Capecchi. (1980). Cell, 22(2): 479-488.
Carrari et al. (2003). Metab. Eng. 5:191-200.
Carrie et al. (2010). Journal of Biological Chemistry 285:36138-36148.
Carter et al. (1980). J. Biol. Chem. 255:4213-4223.
Chacinska et al. (2009). Cell 138:628-644.
Chen et al. (2013). Advanced Drug Delivery Reviews 65:1357-1369.
Cheng et al. (1996). Proceedings of the National Academy of Sciences, 93(8):3636-3641.
Cheng et al. (2005). Biochemical and Biophysical Research Communications 329:966-975.
Chiu et al. (2001). Biochemistry 40:641-650.
Christiansen et al. (1998) Biochemistry-Us 37: 12611-12623.
Clapp (1993). Clinics in perinatology, 20(1):155-168.
Clausen et al. (2000). Proc. Natl. Acad. Sci. U.S.A. 97:3856-361.
Cotton (2009). J. Am. Chem. Soc. 131: 4558-4559.
Cui et al. (2013). Proceedings of the National Academy of Sciences 110, 2052-2057.
Curatti et al. (2006). Proc. Natl. Acad. Sci. U.S.A. 103:5297-5301.
Curatti et al. (2007). Proceedings of the National Academy of Sciences 104(45): 17626-17631.
Curatti and Rubio (2014). Plant Sci 225:130-137.
Curiel et al. (1992). Am J Respir Cell Mol Biol, 6(3): 247-252.
Darshi et al. (2012). Journal of Biological Chemistry 287:39480-39491.
Davis et al. (1996). J. Bacteriol. 178:1445-1450.
Death et al. (2012). Proc. Natl. Acad. Sci. USA 109:17995-17999. de Bruijn (2015). In: Biological Nitrogen Fixation pp. 1087-1101. John Wiley & Sons, Inc.
de Kok et al. (2014). ACS Synth. Biol. 3:97-106.
Dilworth et al. (1988). Biochem. J. 249:745-751.
Dilworth et al. (1993). Biochem. J. 289:395-400.
Dos Santos et al. (2004). Chem Rev. 104:1159-73.
Dos Santos et al. (2012). BMC Genomics 13:162.
Drummond (1985). Biochem. J. 232:891-896.
Dyer et al. (2003). J. Biol. Chem. 278:32150-32156.
Eady (1996). Chem. Rev. 96:3013-3030.
Eglitis et al. (1988). Advances in Experimental Medicine and Biology 241:19-27.
Emerich and Burris, (1978). J. Bacteriol. 134:936-943.
Engler et al. (2014) ACS Synthetic Biology 3(11):839-843.
Fani et al. (2000). J. Mol. Evol. 51:1-11.
Fay et al. (2015) Proc Natl Acad Sci USA 112: 14829-14833.
Fay et al. (2016). Proc. Natl. Acad. Sci. U.S.A. 2016:9504-9508.
Fujimura et al. (1985). Plant Tissue Cult Lett. 2:74-75.
Fukusawa et al. (2015). Molecular and Cellular Proteomics 14:10.1074/mcp.M114.043083, 1113-1126.
Gallie et al. (1987). Nucleic Acids Res. 15:3257-73.
Garfinkel et al. (1983). Cell 27:143-153.
Gavini et al. (1998). Biochemical and Biophysical Research Communications. 244:498-504.
Gavini et al. (2006). Journal of Bacteriology 188:6020-6025.
Geddes et al. (2015). Curr Opin Biotech 32:216-222.
Geigenberger and Fernie (2014). Antioxid Redox Sign 21:1389-1421.
Glaser and Deshi (1999). J Bioenerg Biomembr 31:259-274.
Glibert et al. (2014). Environ Res Lett 9:e105001; doi.org/10.1088/1748-9326/9/10/105001.
Glick et al. (1992). Cell 69:809-822.
Goodwin et al. (1998) Biochemistry-Us 37: 10420-10428.
Good and Beatty (2011). PLoS Biol 9, e1001124.
Graham et al. (1973). Virology 52(2) 456-467.
Grant et al. (1995). J. Agric. Sci., 124 (3): 437-445
Guo et al. (2016). Angewandete Chemie 55:12764-12767
Fu et al. (1994). Biochemistry 1994 33:13455-63.
Hakoyama et al. (2009). Nature 462(7272): 514.
Hellinga (1997). Proc Natl Acad Sci USA. 94:19 10015-10017.
Hernandez et al. (2007). Mol. Microbiol. 63:177-192.
Hirel et al. (1989). Proc. Natl. Acad. Sci. USA 86:8247-8251.
Hinchee et al. (1988).
Homer et al. (1993). J. Bacteriol. 175:4907-4910.
Homer et al. (1995). J. Biol. Chem. 270:24745-24752.
Hoover et al. (1988). Biochemistry 27: 3647-3652.
Horsch et al. (1985). Cold Spring Harbor Symposia on Quantitative Biology 50:433-437.
Howard et al. (1986). J Biol. Chem. 261:772-778
Hu et al. (2004). J. Biol. Chem. 279:54963-54971.
Hu et al. (2005). Proc. Natl. Acad. Sci. U.S.A. 102:3236-3241.
Hu et al, (2006). Proc. Natl. Acad. Sci. U.S.A. 103:17119-17124.
Hu et al. (2008). Biochemistry 47:3973-3981.
Hu and Ribbe (2013). Bba-Bioenergetics 1827:1112-1122.
Hu and Ribbe, (2015). Journal of Biological Inorganic Chemistry 20(2):435-445. doi:10.1007/s00775-014-1225-3
Hu and Ribbe (2016). Annual Review of Biochemistry 85:455-483.
Huang et al. (2009). Plant Physiology 150(3):1272-1285.
Hummel et al. (2007). Metabolomics 75-95.
Hwang et al. (1996). J. Mol. Evol. Nov; 43:536-540.
Igarashi and Seefeldt (2003). Crit. Rev. Biochem. Mol. Biol. 38:351-384.
Jasniewski et al. (2018). Inorganics 6(1):25.
Jimenez-Vicente et al. (2014) FEBS Letters 588:512-516.
Johnson et al. (2005). Biochem. Soc. Trans. 33:90-93.
Joshi (1987). Nucleic Acids Res. 15:6643-6653.
Jouanneau et al. (1995). Biochim. Biophys. Acta 1232:33-42.
Katoh et al. (2013). Mol Biol Evol. 4:772-80.
Kay et al. (1987). Science 236:1299-1302.
Kennedy and Dean, (1992). Mol Gen Genet. 231:494-498.
Kerscher et al. (1997). The Journal of Cell Biology 139:1663-1675.
Khumanthem et al. (2007). Indian Journal of Microbiology, 47:345-352.
Kim and Rees (1994). Biochemistry 33:389-397.
Kimble et al. (1995). Archives of Microbiology 163:259-267.
Klipp et al. (1988). Mol. Gen. Genet. 216:293-302.
Kmiec et al. (2013). PNAS 110: 40 E3761-E3769.
Koon et al. (2004). Proc Natl Acad Sci USA 10:8295-8300.
Lawson and Smith (2002). Met Ions Biol Syst; 39:75-119.
Lee et al. (1998). Biochemical and Biophysical Research Communications 244: 2 498-504.
Lee et al. (2000). J. Bacteriol. 182:7088-7091.
Lee et al. (2012). Plant Cell 24:5037-5057.
Lei et al. (1999). Biochem Biophys Res Commun. 264:186-90.
Lill and Mühlenhoff (2008). Annual Review of Biochemistry 77:669-700.
Lister et al. (2004). 134:777-789.
Lister et al. (2007). Plant Cell, 19:3739-3759.
Lopez-Torrejon et al. (2016). Nature Communications 7:11426.
Lu et al. (1993). Sci China B. 36: 11 1342-51.
Mackenzie and McIntosh (1999). Plant Cell 11:571-585.
Marques et al. (2014). Acta Crystallographica Section F 70(5):669-672.
Masukawa et al. (2007). Appl. Environ. Microbiol. 73:7562-7570.
Matsubara and Saeki (1992). Adv. Inorganic Chem. 38:223-280.
Mayer et al. (1999). J. Mol. Biol. 292:871-891.
McLean and Dixon (1981). Nature, 292:655.
McRose et al. (2017). Frontiers Microbiol. 8:267 doi:10.3389/fmicb.2017.00267.
Medbeffy et al. (1992). The Plant Cell 4: 2 185-192.
Medbeffy et al. (1993). Plant J. 1993 3: 4 619-26.
Merrick and Dixon (1984). Trends Biotechnol 2:162-166.
Mehta and Baross (2006). Science 314:1783-1786.
Millar et al. (2007). Methoods Cell Biol 80:65-90.
Miller and Eady (1988). Biochem. J. 256:429-432.
Miyamoto et al. (1979). Applied and Environmental Microbiology, 37:454-458.
Mueller et al. (2012). Nature 490:254-257.
Mühlenhoff et al. (2003). EMBO J. 22:4815-4825.
Murcha et al. (2004). J Mol Biol 344:443-454.
Murcha et al. (2014). Bba-Gen Subjects 1840:1233-1245.
Niedz et al. (1995). Plant Cell Reports 14: 7 403-6.
Naim et al. (2012). PLoS One 7(12):e52717.
Oldroyd and Dixon (2014) Curr Opin Biotechnol 26:19-24.
Olson et al. (2000). Biochemistry; 39:16213-16219.
Ouzounis et al. (1994). Trends Biochem. Sci. 19:199-200.
Ow et al. (1986). Science 234:856-859.
Paul and Merrick (1987) Eur. J. Biochem. 170:259-265.
Paustian et al. (1990). Biochemistry 29:3515-3522.
Petrova et al. (2000). Biochem. Biophys. Res. Commun. 270:863-867.
Pfanner and Geissler (2001) Nat. Rev. Mol. Cell Biol. 2:339-349.
Prasad et al. (1992). Plant Molecular Biology 18(5):873-885.
Prasher et al. (1985). Trends in Genetics 11: 8 320-3.
Pratte et al. (2006). J. Bacteriol. 188:5806-5811.
Riedel et al. (1995). Eur. J. Biochem. 231:742-746.
Robson and Postgate (1980). Annual Review of Microbiology 34:183-207.
Robson et al. (1986). Nature, 322:388-390.
Robson et al. (1989). EMBO J. 8:1217-1224.
Rockstrom et al. (2009). Nature 461:472-475.
Rodriguez-Quinones et al. (1993). J. Bacteriol. 175:2926-2935.
Roise et al. (1986) The EMBO Journal 5:1327-1334.
Roise and Schatz (1988). J. Biol. Chem. 263:4509-4511.
Rubio et al. (2002). J. Biol. Chem. 277:14299-14305.
Rubio et al. (2004) J Biol Chem 279: 19739-19746.
Rubio and Ludden (2005). J. Bacteriol. 187:405-414.
Rubio and Ludden (2008). Annu Rev Microbiol 62:93-111.
Salomon et al. (1984). EMBO 3:1 141-146.
Santi et al. (2013). Ann Bot 111:743-767.
Schleiff and Soll (2000) Planta 211:449-456.
Schmidt and Skerra (2007). Nat. Protoc. 2:1528-1535
Schmidt et al. (2013). Protein Expr. Purif. 92:54-61.
Schmitz et al. (2001). FEMS Microbiol Lett. 195:97-102.
Schwarz et al. (2016). Nucleic acids research 44(8): e77-e77.
Seefeldt et al. (2009). Annu Rev Biochem 78:701-722.
Serrano et al. (2009). Systematic and Applied Microbiology, 32:1-7.
Shah et al. (1983) J. Biol. Chem. 258:12064-12068.
Shah et al. (1999). J. Bacteriol. 181:2797-2801.
Siddavattam et al. (1993). Mol. Gen. Genet. 239:435-440.
Sippel and Einsle, (2017). Nature Chemical Biology, 13:956. doi:10.1038/nchembio.2428
Sippel et al. (2018). Journal of Biological Inorganic Chemistry 23(7): 1049-1056
Sirrenberg et al. (1996). Nature 384: 6609 582-5.
Smil (2002). Ambio 31:126-131.
Smith et al. (1997). J. Bacteriol. 179:7135-7155.
Smith et al. (2005). Annu. Rev. Biochem. 74:247-281.
Smanski et al. (2014). Nature Biotechnology 32:1241-1249.
Spatzal et al. (2016). Nature communications 7 (2016): 10902.
Stalker et al. (1988). J. Biol. Chem. 263(13):6310-6314.
Staples et al. (2007). J. Bacteriol. 189:7392-7398.
Suh et al. (2003). Journal of Biological Chemistry 278:5353-5360.
Sutton et al. (2008). Environ Pollut 156:583-604.
Temme et al. (2012). Proc. Natl. Acad. Sci. U.S.A. 109(18):7085-7090.
Tezcan et al. (2005). Science 309:1377-1380.
Thiel et al. (1995) Proc Natl Acad Sci USA 92: 9358-9362.
Thiel et al. (1997). J. Bacteriol. 179:5222-5225.
Thillet et al. (1988). J. Biol. Chem 263(25):12500-12508
Thomas et al. (1966). Biochemistry 5(8):2513-2516.
Toriyama et al. (1986). Theor. Appl. Genet. 73:16-19
Verhasselt et al. (1995). Yeast 11(10):961-966.
von Heijne (1986). EMBO J. 5:1335-1342.
Wagner et al. (1992). Proc. Nail. Acad. Sci. U.S.A. 89:6099-6103
Wahlund and Madigan, (1993). J. Bacteriol. 175: 474-478.
Wang et al. (2013). PLoS Genet 9, e1003865.
Waterhouse et al. (2018). Nucleic Acids Res. 46(W1), W296-W303.
Weber et al. (2011) PloS one. 6(2), pp.e16765.
Wiig et al. (2011) Proc Natl Acad Sci USA 108: 8623-8627.
Wisniewski et al. (2011) Anal Biochem. 410:307-9.
Wood et al. (2009). Plant Biotechnol J. 7:914-924.
Xia et al. (2009). Nucleic acids research 37(suppl_2): W652-W660.
Xiao et al. (2010). Biochemistry 49:5588-5599.
Yang et al. (2014). Proc. Natl. Acad. Sci. U.S.A. 111:E3718-E3725.
Yang et al. (2010). BMC Plant Biology, 10. doi:10.1186/1471-2229-10-231.
Yang et al. (2017). Proc Natl Acad Sci USA 114:E2460-E2465.
Yang et al. (2018) Proc Natl Acad Sci USA doi/10.1073/pnas.1804992115
Yates (1972) FEBS Lett 27:63-67.
Yoneda et al. (2012). Int. J. Systematic Evol. Biol. 62:1692-1697.
Yuvaniyama et al. (2000) Proc. Natl. Acad. Sci. USA 97:599-604.
Zhang and Glaser (2002). Trends Plant Sci 7:14-21.
Zhang et al. (2009). Progress in Natural Science 19:1197-1200.
Zhang et al. (2016). Biogeochemistry 127, 189-198. doi: 10.1007/s10533-016-0188-6.
Zhang and Wang (2013). PLoS One 8(7). doi:10.1371/journal.pone.0068491.
Zheng et al. (1994)
Zheng et al. (1997). J. Bacteriol. 179:5963-5966.
Zheng et al. (2018). Nature Microbiology, 3:281-286. doi:10.1038/s41564-017-0091-5.

1. A plant cell comprising mitochondria and exogenous polynucleotides which encode at least 8 or at least 9 Nif fusion polypeptides, wherein the exogenous polynucleotides each comprise a promoter which is operably linked to a nucleotide sequence which encodes one of the Nif fusion polypeptides and which expresses the nucleotide sequence in the plant cell, wherein each Nif fusion polypeptide independently comprises a mitochondrial targeting peptide (MTP), wherein the Nif fusion polypeptides comprise (i) NifH, NifB, NifF, NifJ, NifS, NifU and NifV fusion polypeptides and either (ii) a NifD fusion polypeptide and a NifK fusion polypeptide or (iii) a NifD-linker-NifK fusion polypeptide which comprises a NifD sequence having a C-terminus, an oligopeptide linker and a NifK sequence having a N-terminus, wherein the oligopeptide linker is translationally fused to the C-terminus of the NifD sequence and the N-terminus of the NifK sequence, wherein mitochondrial processing protease (MPP)-cleaved products of at least the NifH, NifF, NifS and NifU fusion polypeptides are each at least partially soluble in mitochondria of a plant cell, wherein MPP-cleaved products of the NifD and NifK fusion polypeptides of (ii) if present in the plant cell are at least partially soluble in mitochondria of a plant cell, or a MPP-cleaved product of the NifD-linker-NifK fusion polypeptide of (iii) if present in the plant cell is at least partially soluble in mitochondria of a plant cell, and wherein the NifV fusion polypeptide and/or a MPP-cleaved product thereof produces homocitrate in the plant cell and is at least partially soluble in mitochondria of a plant cell.

2. A plant cell comprising mitochondria and exogenous polynucleotides which encode at least 2, at least 3, at least 4, at least 5 or at least 6 Nif fusion polypeptides, wherein the exogenous polynucleotides each comprise a promoter which is operably linked to a nucleotide sequence which encodes one of the Nif fusion polypeptides and which expresses the nucleotide sequence in the plant cell, wherein each Nif fusion polypeptide independently comprises a mitochondrial targeting peptide (MTP), wherein the Nif fusion polypeptides comprise (i) one or more than one or all of NifW, NifX, NifY, and NifZ fusion polypeptides, and either (ii) a NifD fusion polypeptide and a NifK fusion polypeptide or (iii) a NifD-linker-NifK fusion polypeptide which comprises a NifD sequence having a C-terminus, an oligopeptide linker and a NifK sequence having a N-terminus, wherein the oligopeptide linker is translationally fused to the C-terminus of the NifD sequence and the N-terminus of the NifK sequence, wherein mitochondrial processing protease (MPP)-cleaved products of at least the NifW, NifX, NifY and NifZ fusion polypeptides if present in the plant cell are each at least partially soluble in mitochondria of a plant cell, wherein either MPP-cleaved products of the NifD and NifK fusion polypeptides of (ii) if present in the plant cell are at least partially soluble in mitochondria of a plant cell, or a MPP-cleaved product of the NifD-linker-NifK fusion polypeptide of (iii) if present in the plant cell is at least partially soluble in mitochondria of a plant cell, and wherein the MPP-cleaved products of the NifD fusion polypeptide and NifK fusion polypeptide of ii) or the MPP-cleaved product of the NifD-linker-NifK fusion polypeptide of iii) is present in the plant cell in greater amount than the amount of the MPP-cleaved products of the NifD fusion polypeptide and NifK fusion polypeptide or the MPP-cleaved product of the NifD-linker-NifK fusion polypeptide present in a corresponding plant cell lacking the exogenous polynucleotides encoding the one or more than one or all of NifW, NifX, NifY and NifZ fusion polypeptides of (i).

3. A plant cell comprising mitochondria and exogenous polynucleotides which encode at least 5, at least 6, at least 7, at least 8 or at least 9 Nif fusion polypeptides, wherein the exogenous polynucleotides each comprise a promoter which is operably linked to a nucleotide sequence which encodes one of the Nif fusion polypeptides and which expresses the nucleotide sequence in the plant cell, wherein each Nif fusion polypeptide independently comprises a mitochondrial targeting peptide (MTP), wherein the Nif fusion polypeptides comprise (i) NifH, NifS and NifU fusion polypeptides and optionally a NifM polypeptide, (ii) one or more than one or all of NifW, NifX, NifY, and NifZ fusion polypeptides and either (iii) a NifD fusion polypeptide and a NifK fusion polypeptide or (iv) a NifD-linker-NifK fusion polypeptide which comprises a NifD sequence having a C-terminus, an oligopeptide linker and a NifK sequence having a N-terminus, wherein the oligopeptide linker is translationally fused to the C-terminus of the NifD sequence and the N-terminus of the NifK sequence, wherein mitochondrial processing protease (MPP)-cleaved products of the NifS and NifU fusion polypeptides are at least partially soluble in mitochondria of a plant cell, wherein MPP-cleaved products of the NifW, NifX, NifY and NifZ fusion polypeptides, if present in the plant cell, are at least partially soluble in mitochondria of a plant cell, wherein MPP-cleaved products of the NifD and NifK fusion polypeptides of (iii), if present in the plant cell, are at least partially soluble in mitochondria of a plant cell, wherein a MPP-cleaved product of the NifD-linker-NifK fusion polypeptide of (iv), if present in the plant cell, is at least partially soluble in mitochondria of a plant cell, and wherein either the MPP-cleaved products of the NifD fusion polypeptide and NifK fusion polypeptide of iii) or the MPP-cleaved product of the NifD-linker-NifK fusion polypeptide of iv) are present in the plant cell as a complex with P-cluster.

4. The plant cell according to any one of claims 1 to 3, wherein the plant cell comprises a NifH fusion polypeptide which is an AnfH fusion polypeptide, wherein the NifD fusion polypeptide if present is an AnfD fusion polypeptide, the NifK fusion polypeptide if present is an AnfK fusion polypeptide, the NifD-linker-NifK fusion polypeptide if present is an AnfD-linker-AnfK fusion polypeptide, and the plant cell further comprises an exogenous polynucleotide which encodes an AnfG fusion polypeptide which comprises a MTP, wherein the exogenous polynucleotide which encodes the AnfG fusion polypeptide comprises a promoter which is operably linked to a nucleotide sequence which encodes the AnfG fusion polypeptide and which expresses said nucleotide sequence in the plant cell, and wherein a MPP-cleaved product of the AnfG fusion polypeptide is at least partially soluble in mitochondria of a plant cell.

5. A plant cell comprising mitochondria and exogenous polynucleotides which encode at least 2, at least 3 or 4 Anf fusion polypeptides, wherein the exogenous polynucleotides each comprise a promoter which is operably linked to a nucleotide sequence which encodes one of the Anf fusion polypeptides and which expresses the nucleotide sequence in the plant cell, wherein each Anf fusion polypeptide independently comprises a mitochondrial targeting peptide (MTP), wherein the Anf fusion polypeptides comprise (i) an AnfG fusion polypeptide or AnfG and AnfH fusion polypeptides, and either (ii) an AnfD fusion polypeptide and an AnfK fusion polypeptide or (iii) an AnfD-linker-AnfK fusion polypeptide which comprises an AnfD sequence having a C-terminus, an oligopeptide linker and an AnfK sequence having a N-terminus, wherein the oligopeptide linker is translationally fused to the C-terminus of the AnfD sequence and the N-terminus of the AnfK sequence, wherein mitochondrial processing protease (MPP)-cleaved products of at least the AnfG and AnfH fusion polypeptides if present in the plant cell are each at least partially soluble in mitochondria of a plant cell, wherein either MPP-cleaved products of the AnfD and AnfK fusion polypeptides of (ii) if present in the plant cell are at least partially soluble in mitochondria of a plant cell, or a MPP-cleaved product of the AnfD-linker-AnfK fusion polypeptide of (iii) if present in the plant cell is at least partially soluble in mitochondria of a plant cell, and wherein the MPP-cleaved products of the AnfD fusion polypeptide and the AnfK fusion polypeptide of ii) or the MPP-cleaved product of the AnfD-linker-AnfK fusion polypeptide of iii) if present in the plant cell form a protein complex in the plant cell with the MPP-cleaved product of the AnfG fusion polypeptide.

6. The plant cell according to any one of claims 1 to 4, wherein the NifD fusion polypeptide or the NifD-linker-NifK fusion polypeptide is present in the plant cell and is (a) resistant to protease cleavage at a site within an amino acid sequence corresponding to amino acids 97-100 of SEQ ID NO:18 and/or (b) comprises an amino acid sequence other than RRNY (SEQ ID NO:101) at positions corresponding to amino acids 97-100 of SEQ ID NO:18.

7. A plant cell comprising mitochondria and an exogenous polynucleotide which encodes a NifV polypeptide (NV), wherein the exogenous polynucleotide comprises a promoter which is operably linked to a nucleotide sequence which encodes the NV and which expresses said nucleotide sequence in the plant cell, wherein the NV produces homocitrate in the plant cell and is at least partially soluble in mitochondria of a plant cell, wherein the exogenous polynucleotide is preferably integrated into the nuclear genome of the plant cell and/or is expressed in the nucleus of the plant cell, and optionally wherein the NV comprises a mitochondrial targeting peptide (MTP).

8. A plant cell comprising an exogenous polynucleotide which encodes a NifD polypeptide (ND) which is (a) resistant to protease cleavage at a site within an amino acid sequence corresponding to amino acids 97-100 of SEQ ID NO:18, and/or (b) comprises an amino acid sequence other than RRNY (SEQ ID NO:101) at positions corresponding to amino acids 97-100 of SEQ ID NO:18, wherein the exogenous polynucleotide comprises a promoter which is operably linked to a nucleotide sequence which encodes the ND and which expresses said nucleotide sequence in the plant cell, and wherein the NifD polypeptide preferably comprises a MTP.

9. The plant cell of claim 8, which comprises an exogenous polynucleotide which encodes a NifK polypeptide (NK), wherein the exogenous polynucleotide which encodes the NK comprises a promoter which is operably linked to a nucleotide sequence which encodes the NK and which expresses said nucleotide sequence in the plant cell, wherein the ND has a C-terminus and the NK has an N-terminus, and wherein either (i) the NK comprises a mitochondrial targeting peptide (MTP), or (ii) the ND and NK are translationally fused as a NifD-linker-NifK fusion polypeptide which comprises an oligopeptide linker, wherein the oligopeptide linker is translationally fused to the C-terminus of the ND and the N-terminus of the NK.

10. The plant cell of claim 8 or claim 9, which further comprises an exogenous polynucleotide which encodes a NifH fusion polypeptide (NH), wherein the exogenous polynucleotide which encodes the NH comprises a promoter which is operably linked to a nucleotide sequence which encodes the NH and which expresses said nucleotide sequence in the plant cell, wherein the NH comprises a mitochondrial targeting peptide (MTP), and preferably wherein the NH and/or a MPP-cleaved product thereof is at least partially soluble in mitochondria of a plant cell.

11. The plant cell of any one of claims 8 to 10, wherein a MPP-cleaved product of at least one or more of the polypeptides is at least partially soluble in mitochondria of a plant cell, preferably wherein a MPP-cleaved product of each of the NifD, NifK and NifD-linker-NifK fusion polypeptides, if present in the plant cell, and the NifH polypeptide is at least partially soluble in mitochondria of a plant cell.

12. A plant cell comprising an exogenous polynucleotide which encodes a NifH fusion polypeptide (NH), wherein the exogenous polynucleotide comprises a promoter which is operably linked to a nucleotide sequence which encodes the NH and which expresses said nucleotide sequence in the plant cell, wherein the NH comprises a mitochondrial targeting peptide (MTP), wherein a MPP-cleaved product of the NH is at least partially soluble in mitochondria of a plant cell, and optionally wherein the exogenous polynucleotide is integrated into the nuclear genome of the plant cell and/or is expressed in the nucleus of the plant cell.

13. The plant cell according to any one of claims 1 to 12, which further comprises an exogenous polynucleotide which encodes a NifM polypeptide (NM), wherein the exogenous polynucleotide which encodes the NM comprises a promoter which is operably linked to a nucleotide sequence which encodes the NM and which expresses said nucleotide sequence in the plant cell, and wherein the NM optionally comprises a mitochondrial targeting peptide (MTP).

14. The plant cell according to any one of claims 2 or 4 to 13, wherein the plant cell comprises exogenous polynucleotides which encode NifS and NifU fusion polypeptides, wherein the exogenous polynucleotides each comprise a promoter which is operably linked to a nucleotide sequence which encodes one of the Nif fusion polypeptides and which expresses the nucleotide sequence in the plant cell, and wherein the NifS and NifU fusion polypeptides each comprise a mitochondrial targeting peptide (MTP).

15. The plant cell according to any one of claims 7 to 11 or claim 13 or 14, wherein each Nif polypeptide is produced in the plant cell as a Nif fusion polypeptide comprising a mitochondrial targeting peptide (MTP), wherein each MTP is independently the same or different, preferably wherein the MTP is at the N-terminus of at least one or more than one or all of the Nif fusion polypeptides.

16. The plant cell according to any one of claims 1 to 15, wherein each Nif fusion polypeptide produced in the plant cell is independently cleaved by MPP either (i) within the MTP sequence to yield a MPP-cleaved Nif polypeptide, whereby the MPP-cleaved Nif polypeptide comprises, at its N-terminal end, a C-terminal peptide from the MTP (scar peptide), or (ii) immediately after the MTP whereby the MPP-cleaved Nif polypeptide does not comprise a C-terminal peptide from the MTP.

17. The plant cell of claim 15 or claim 16, wherein each MTP is independently cleaved in the plant cell with an efficiency of at least 50%, and/or wherein each cleaved Nif polypeptide is independently present in the plant cell at a greater level than a corresponding uncleaved Nif fusion polypeptide, preferably at a ratio of greater than 1:1, 2:1 or 3:1.

18. The plant cell according to any one of claims 1 to 17, wherein each Nif fusion polypeptide is at least partially cleaved in its MTP sequence in the plant cell to produce a MPP-cleaved Nif polypeptide, wherein each MPP-cleaved Nif polypeptide independently comprises a peptide (scar peptide) of 1 to 45 amino acids in length, preferably 1 to 20 amino acids, more preferably 1 to 11 amino acids or 11 to 20 amino acids derived from the MTP sequence, translationally fused at the N-terminal end of the MPP-cleaved Nif polypeptide.

19. The plant cell according to any one of claims 1 to 18, which further comprises an exogenous polynucleotide which encodes a ferredoxin fusion polypeptide, preferably a FdxN fusion polypeptide, wherein the exogenous polynucleotide which encodes the ferredoxin fusion polypeptide comprises a promoter which is operably linked to a nucleotide sequence which encodes the ferredoxin fusion polypeptide and which expresses said nucleotide sequence in the plant cell, and wherein the ferredoxin fusion polypeptide comprises a mitochondrial targeting peptide (MTP).

20. The plant cell of claim 19, wherein a MPP-cleaved product of the ferredoxin fusion polypeptide is at least partially soluble in mitochondria of a plant cell, and preferably wherein the exogenous polynucleotide is integrated into the nuclear genome of the plant cell and/or is expressed in the nucleus of the plant cell.

21. The plant cell according to any one of claims 1 to 20, comprising a NifD-linker-NifK fusion polypeptide comprising, in order, a NifD amino acid sequence (ND), an oligopeptide linker and a NifK polypeptide (NK) amino acid sequence, wherein the oligopeptide linker has a length of 8-50 residues, preferably 16-50 residues in length, more preferably about 26 or about 30 residues in length, or most preferably is 30 residues in length, which is translationally fused to the ND and NK.

22. The plant cell according to any one of claims 1 to 21, wherein each Nif fusion polypeptide is cleaved in the plant cell to produce a Nif polypeptide which is a functional Nif polypeptide.

23. The plant cell according to any one of claims 1 to 7 or 12 to 22, comprising an exogenous polynucleotide which encodes a NifD fusion polypeptide (ND) or a NifD-linker-NifK fusion polypeptide, wherein the ND or the NifD-linker-NifK fusion polypeptide comprises an amino acid sequence other than RRNY (SEQ ID NO:101) at positions corresponding to amino acids 97-100 of SEQ ID NO:18, and wherein the ND or the NifD-linker-NifK fusion polypeptide preferably comprises an amino acid other than tyrosine (Y) at a position corresponding to amino acid 100 of SEQ ID NO:18.

24. The plant cell of claim 23, wherein the ND or the NifD-linker-NifK fusion polypeptide comprises a glutamine (Q) or lysine (K) at the position corresponding to amino acid 100 of SEQ ID NO:18, or a leucine (L) or methionine (M) or a phenylalanine (F) at the position corresponding to amino acid 100 of SEQ ID NO:18.

25. The plant cell according to any one of claims 1 to 24, comprising an exogenous polynucleotide which encodes a NifK fusion polypeptide or NifD-linker-NifK fusion polypeptide, wherein the NifK fusion polypeptide or the NifD-linker-NifK fusion polypeptide has a C-terminal amino acid sequence which is the same as a C-terminal amino acid sequence of a wild-type NifK polypeptide.

26. The plant cell of claim 25, wherein the NifK fusion polypeptide or NifD-linker-NifK fusion polypeptide has an amino acid sequence whereby the last four amino acids of the sequence are the same as the last four amino acids of a wild-type NifK polypeptide.

27. The plant cell according to any one of claims 1 to 26, comprising an exogenous polynucleotide which encodes a AnfD-linker-AnfK fusion polypeptide, wherein the AnfD-linker-AnfK fusion polypeptide comprises an AnfD sequence which has a C-terminus, an oligopeptide linker and an AnfK sequence which comprises an N-terminus, wherein the oligopeptide linker is translationally fused to the C-terminus of the AnfD sequence and the N-terminus of the AnfK sequence, wherein the oligopeptide linker has a length of at least about 20 amino acids, at least about 30 amino acids, at least about 40 amino acids, about 20 amino acids to about 70 amino acids, about 30 amino acids to about 70 amino acids, about 30 amino acids to about 60 amino acids, about 30 amino acids to about 50 amino acids, about 25 amino acids, about 30 amino acids, about 35 amino acids, about 40 amino acids, about 45 amino acids, about 46 amino acids, about 50 amino acids or about 55 amino acids.

28. The plant cell of claim 5, further comprising one or more exogenous polynucleotides encoding one or more Nif fusion polypeptides as defined in any one of claims 1 to 4 or 6 to 27.

29. The plant cell of claim 7, further comprising one or more exogenous polynucleotides encoding one or more Nif fusion polypeptides as defined in any one of claims 1 to 6 or 8 to 27.

30. The plant cell of claim 8, further comprising one or more exogenous polynucleotides encoding one or more Nif fusion polypeptides as defined in any one of claims 1 to 7 or 9 to 27.

31. The plant cell of claim 12, further comprising one or more exogenous polynucleotides encoding one or more Nif fusion polypeptides as defined in any one of claims 1 to 11 or 13 to 27.

32. The plant cell according to any one of claims 1 to 31, wherein at least one or more than one or preferably all of the exogenous polynucleotides are integrated into the nuclear genome of the plant cell and/or are expressed in the nucleus of the plant cell.

33. The plant cell according to any one of claims 1 to 32, wherein at least one of the Nif fusion polypeptides comprises an MTP which is about 51 amino acids in length from a F1-ATPase γ-subunit polypeptide.

34. A plant or a part thereof comprising a plant cell according to any one of claims 1 to 33, or which is transgenic for the exogenous polynucleotides encoding the Nif fusion polypeptides defined in any one of claims 1 to 33.

35. The plant or part thereof of claim 34, wherein one or more than one or all of the exogenous polynucleotides are expressed in roots of the plant, preferably expressed at a greater level in the roots of the plant than in leaves of the plant.

36. The plant or part thereof of claim 34 or claim 35, which is a cereal plant, preferably a wheat, rice, maize, triticale, oat or barley plant, or part thereof.

37. A NifD fusion polypeptide comprising a mitochondrial targeting peptide (MTP) translationally fused to a NifD polypeptide (ND), or a cleaved product thereof which comprises the ND and optionally a scar peptide, wherein the NifD fusion polypeptide or the cleaved product thereof is (a) resistant to protease cleavage at a site within an amino acid sequence corresponding to amino acids 97-100 of SEQ ID NO:18 and/or (b) comprises an amino acid sequence other than RRNY (SEQ ID NO:101) at positions corresponding to amino acids 97-100 of SEQ ID NO:18.

38. The NifD fusion polypeptide of claim 37, which comprises an oligopeptide linker and a NifK polypeptide (NK) which are translationally fused as a NifD-linker-NifK fusion polypeptide, wherein the ND comprises a C-terminus and the NK comprises an N-terminus, wherein the oligopeptide linker is translationally fused to the C-terminus of the ND and the N-terminus of the NK.

39. A cleaved product of the NifD fusion polypeptide of claim 38, wherein the cleaved product comprises the ND, an oligopeptide linker and the NK, wherein the oligopeptide linker is translationally fused to the C-terminus of the ND and the N-terminus of the NK.

40. The NifD fusion polypeptide of claim 37 or 38, or the cleaved product of claim 39, which is at least partially soluble in mitochondria of a plant cell when the NifD fusion polypeptide is produced in the plant cell.

41. The NifD fusion polypeptide of claim 37 or 40, the NifD-linker-NifK fusion polypeptide of claim 33, or the cleaved product of claim 34, wherein the NifD fusion polypeptide is an AnfD fusion polypeptide, the NK is an AnfK polypeptide, and the NifD-linker-NifK fusion polypeptide is an AnfD-linker-AnfK fusion polypeptide.

42. A NifK fusion polypeptide comprising a mitochondrial targeting peptide (MTP) translationally fused to a NifK polypeptide (NK), wherein the NifK fusion polypeptide or a cleaved product thereof is at least partially soluble in mitochondria of a plant cell when the NifK fusion polypeptide or the cleaved product thereof is produced in the plant cell.

43. A cleaved product of the NifK fusion polypeptide of claim 42, which comprises the NK and optionally a scar peptide, wherein the cleaved product is at least partially soluble in mitochondria of a plant cell when the cleaved product is produced in the plant cell.

44. The NifK fusion polypeptide of claim 42 or the cleaved product of claim 43, wherein the NK is an AnfK polypeptide (AK).

45. The NifD fusion polypeptide or cleaved product thereof of any one of claims 38 to 41 or the NifK fusion polypeptide or cleaved product thereof of any one of claims 42 to 44, wherein the NifK polypeptide has a C-terminal amino acid sequence which is the same as the C-terminal amino acid sequence of a wild-type NifK polypeptide.

45. An AnfD fusion polypeptide comprising a mitochondrial targeting peptide (MTP) and an AnfD polypeptide (AD), or a cleaved product thereof comprising the AD and optionally a scar peptide, preferably which is at least partially soluble in mitochondria of a plant cell when the AnfD fusion polypeptide or the cleaved product thereof is produced in the plant cell.

46. An AnfH fusion polypeptide comprising a mitochondrial targeting peptide (MTP) and an AnfH polypeptide (AH), or a cleaved product thereof comprising the AH and optionally a scar peptide, preferably which is at least partially soluble in mitochondria of a plant cell when the AnfH fusion polypeptide or the cleaved product thereof is produced in the plant cell.

48. An AnfG fusion polypeptide comprising a mitochondrial targeting peptide (MTP) and an AnfG polypeptide (AG), or a cleaved product thereof comprising the AG and optionally a scar peptide, preferably which is at least partially soluble in mitochondria of a plant cell when the AnfG fusion polypeptide or the cleaved product thereof is produced in the plant cell.

49. An AnfD-linker-AnfK fusion polypeptide or a cleaved product thereof, comprising an AnfD polypeptide (AD), an oligopeptide linker and an AnfK polypeptide (AK) which are translationally fused, wherein the AD comprises an N-terminus and a C-terminus, and the AK comprises an N-terminus, wherein the oligopeptide linker is translationally fused to the C-terminus of the AD and the N-terminus of the AK, preferably wherein the fusion polypeptide comprises a mitochondrial targeting peptide (MTP) or the cleaved product comprises a scar peptide translationally fused to the N-terminus of the AD.

50. A protein complex comprising (i) the cleaved product according to any one of claims 39 to 41, (ii) the cleaved product of claim 43 or 44, and optionally (iii) an Fe—S cluster, preferably a P-cluster.

51. A protein complex comprising (i) the cleaved products of claims 44 and 46, and optionally the cleaved product of claim 48, or (ii) the cleaved products of claims 48 and 49, and optionally (iii) an Fe—S cluster, preferably a P-cluster.

52. The protein complex of claim 50 or claim 51 which is in a plant cell, preferably in a mitochondrion of the plant cell.

53. An isolated or recombinant NifV polypeptide, or a NifV fusion polypeptide comprising a mitochondrial targeting peptide (MTP) translationally fused to a NifV polypeptide (NV), or a cleaved product thereof which comprises the NV and optionally a scar peptide, wherein the NifV polypeptide and/or the NifV fusion polypeptide and/or the cleaved product thereof is at least partially soluble in a plant cell when produced in the plant cell, preferably is at least partially soluble in mitochondria of the plant cell.

54. The polypeptide of claim 53 which is capable of producing homocitrate in a plant cell, preferably in mitochondria of a plant cell.

55. A NifH fusion polypeptide comprising a mitochondrial targeting peptide (MTP) translationally fused to a NifH polypeptide (NH), or a cleaved product thereof which comprises the NH and optionally a scar peptide, wherein the NifH fusion polypeptide and/or the cleaved product thereof is at least partially soluble in mitochondria of a plant cell.

56. The NifH fusion polypeptide or cleavage product of claim 55 which is bound to one or two Fe—S clusters, preferably one or two Fe₄-54 clusters.

57. A polynucleotide encoding the fusion polypeptide of any one of claims 37, 38, 40 to 42, 44 to 49 or 53 to 56.

58. The polynucleotide of claim 57, wherein a polypeptide coding region of the polynucleotide has been codon-modified for expression in a plant cell, relative to a corresponding polypeptide coding region of a naturally-occurring polynucleotide in a bacterium.

59. The polynucleotide of claim 57 or claim 58 further comprising a promoter operably linked to the polynucleotide encoding the polypeptide.

60. The polynucleotide according to any one of claims 57 to 59 which is present in a plant cell, a yeast cell or a bacterial cell.

61. The polynucleotide of claim 60, which is integrated into the nuclear genome of the plant cell and/or expressed in the nucleus of the plant cell.

62. A vector comprising the polynucleotide according to any one of claims 57 to 61.

63. The vector of claim 62 comprising polynucleotides which encode at least 3, at least 4, or at least 5 Nif fusion polypeptides.

64. A vector comprising polynucleotides which encode at least 3, at least 4, or at least of the Nif fusion polypeptides defined in any one of claims 1 to 33 or the fusion polypeptides of any one of claims 37, 38, 40 to 42, 44 to 49 or 53 to 56.

65. The vector of claim 64, which comprises polynucleotides encoding
a) the NifD fusion polypeptide and the NifK fusion polypeptide, or the NifD-linker-NifK fusion polypeptide; and
b) the NifH fusion polypeptide and the NifV fusion polypeptide; and
c) optionally, the AnfG fusion polypeptide and/or the ferredoxin fusion polypeptide.

66. The vector of claim 64, which comprises polynucleotides encoding
a) the NifF, NifJ, NifU and NifB fusion polypeptides and optionally the NifS fusion polypeptide; and/or
b) the NifW, NifX, NifY and NifZ fusion polypeptides.

67. A cell, preferably a plant cell, comprising a fusion polypeptide or cleaved product according to any one of claims 37 to 49 or 53 to 56, or a combination of two or more of said fusion polypeptides or cleaved products, a protein complex according to any one of claims 50 to 52, and/or a polynucleotide according to any one of claims 57 to 61 or a vector according to claims 62 to 66.

68. The cell of claim 67, wherein the fusion polypeptide or cleaved product, or combination of fusion polypeptides or cleavage products, or protein complex, is in mitochondria of the cell.

69. Use of a polynucleotide according to any one of claims 57 to 61, and/or a vector according to any one of claims 62 to 66, for producing a transgenic plant cell.

70. A method of producing homocitrate in a plant cell, the method comprising expressing the recombinant NifV polypeptide or the NifV fusion polypeptide of claim 53 or claim 54 in the plant cell, wherein the recombinant NifV polypeptide or the NifV fusion polypeptide, and/or a cleaved product thereof, produces homocitrate in the plant cell.

71. The method of claim 70, further comprising introducing a polynucleotide encoding the recombinant NifV polypeptide or the NifV fusion polypeptide of claim 53 or claim 54 into the plant cell.

72. Use of the NifV polypeptide of claim 53 or claim 54 for producing homocitrate in a plant cell.

73. A method of increasing the amount of a NifD, NifK or NifD-linker-NifK fusion polypeptide in a plant cell, the method comprising expressing one or more or all of NifW, NifX, NifY and NifZ fusion polypeptides in the plant cell, wherein each Nif fusion polypeptide independently comprises a mitochondrial targeting peptide (MTP), wherein the amount of the NifD, NifK or NifD-linker-NifK fusion polypeptide in the plant cell is increased relative to a corresponding plant cell not expressing one or more or all of the NifW, NifX, NifY and NifZ fusion polypeptides.

74. The method of claim 73, further comprising
i) introducing one or more polynucleotides encoding the NifD, NifK or NifD-linker-NifK fusion polypeptide into the plant cell; and
ii) introducing one or more polynucleotides encoding one or more or all of the NifW, NifX, NifY and NifZ fusion polypeptides into the plant cell.

75. A method of increasing the amount of a NifY polypeptide in a plant cell, the method comprising expressing one or more or all of NifW, NifX and NifZ fusion polypeptides in the plant cell, wherein each Nif fusion polypeptide independently comprises a mitochondrial targeting peptide (MTP), wherein the amount of the NifY polypeptide in the plant cell is increased relative to a corresponding plant cell not expressing one or more or all of the NifW, NifX and NifZ fusion polypeptides.

76. The method of claim 75, further comprising
i) introducing a polynucleotide encoding a NifY fusion polypeptide into the plant cell; and
ii) introducing one or more polynucleotides encoding the one or more or all of the NifW, NifX and NifZ fusion polypeptides into the plant cell.

77. Use of one or more polynucleotides encoding one or more or all of NifW, NifX and NifZ fusion polypeptides to increase the amount of a NifY polypeptide in a plant cell.

78. A method of producing a transgenic plant, the method comprising the steps of
i) introducing one or more polynucleotides according to any one of claims 57 to 61, and/or one or more vectors according to any one of claims 62 to 66, into a cell of a plant,
ii) from the cell of step i), regenerating a transgenic plant according to any one of claims 34 to 36, and
iii) optionally, producing transgenic seed and/or progeny plants from the transgenic plant regenerated in step ii).

79. A method of producing transgenic seed, comprising
i) harvesting seed from the transgenic plant according to any one of claims 34 to 36, and/or
ii) harvesting seed from one or more transgenic progeny plants produced by the method of claim 78.

80. The plant part of any one of claims 34 to 36, which is a seed.

81. A method of producing flour, wholemeal, starch, oil, seed meal or other product obtained from seed, the method comprising;
a) obtaining the seed of claim 80, and/or
b) extracting the flour, wholemeal, starch, oil or other product, or producing the seed meal from the seed of claim 80.

82. A product produced from the transgenic plant or part thereof according to any one of claims 34 to 36 and/or the seed of claim 80, comprising the polypeptide or cleavage product of any one of claims 37 to 49 or 53 to 56 or the polynucleotide according to any one of claims 57 to 61.

83. A method of preparing a food product, the method comprising mixing seed of claim 80, or flour, wholemeal, starch, oil or other product from the seed, with another food ingredient, or processing the seed or flour or wholemeal, preferably by milling, cracking, polishing, flaking, parboiling, cooking or baking the seed or a composition comprising the seed and/or flour or wholemeal obtained from the seed.
